(19) United States
(12) Patent Application Publication
(10) Pub. No.: US 2013/0023541 A1
(43) Pub. Date: Jan. 24, 2013

(54) VOLTAGE-GATED SODIUM CHANNEL BLOCKERS

(71) Applicants: Jeffrey Charles Boehm, King of Prussia, PA (US); Roderick S. Davis, King of Prussia, PA (US); Jeffrey K. Kerns, King of Prussia, PA (US); Guoliang Lin, King of Prussia, PA (US); Hong Nie, King of Prussia, PA (US)

(72) Inventors: Jeffrey Charles Boehm, King of Prussia, PA (US); Roderick S. Davis, King of Prussia, PA (US); Jeffrey K. Kerns, King of Prussia, PA (US); Guoliang Lin, King of Prussia, PA (US); Hong Nie, King of Prussia, PA (US)

(73) Assignee: Glaxo Group Limited

(21) Appl. No.: 13/522,183

(22) PCT Filed: Jan. 13, 2011
(86) PCT No.: PCT/US11/21114

§ 371 Date: Jul. 13, 2012

Related U.S. Application Data
(60) Provisional application No. 61/295,138, filed on Jan. 14, 2010.
(60) Provisional application No. 61/317,005, filed on Mar. 24, 2010.

(51) Int. Cl. A61K 31/496; A61P 11/00; C07D 401/04

(52) U.S. Cl. 514/253.13; 544/365

(57) ABSTRACT

The present invention relates to voltage-gated sodium channel blocker intermediates, compounds and dimers, corresponding pharmaceutical compositions, compound preparation and treatment methods for respiratory or respiratory tract diseases.

### FIELD OF THE INVENTION

The present invention relates to voltage-gated sodium channel blocker compounds, corresponding pharmaceutical compositions, compound preparation and treatment methods for respiratory and respiratory tract diseases.

### BACKGROUND OF THE INVENTION

Sodium channels play a significant role in the neuronal network by transmitting electrical impulses rapidly throughout cells and cell networks, which aid in coordinating higher processes ranging from locomotion to cognition in mammals.

In general, sodium channels are described in the art as large transmembrane proteins, which are able to switch between different states to enable selective permeability for sodium ions. For such a process, an action potential, a short-lasting event in which the electrical membrane potential of a cell rapidly rises and falls, is needed to depolarize transmembranes, in which sodium channels are voltage-gated.

Voltage-gated sodium channels are responsible for generation of the action potentials of axonal nerve fibers via fast, selective transport of sodium ions across cell membranes resulting to rapid transmission of depolarizing impulses throughout cells and cell networks. Thus, voltage-gated sodium channels are responsible for initial phase of action potential, which is a wave of electrical depolarization usually initiated at the soma of the neuron and propagated along the nerve axon to the terminals. At the terminals, the action potential triggers the influx of calcium and the release of neurotransmitter.

Research in this area has shown that voltage-gated sodium channels could be targeted, either selectively or in combination with other cellular processes, for the treatment of different diseases, which include, but are not limited to, for example, treatment of stroke, epilepsy and several types of neuropathic pain.

A key feature of these drugs is their use-dependent mechanism of action. The mechanism by which sodium channels are able to inactivate has been the subject of extensive study. It is clear that these channels are able to inactivate through both a fast (milliseconds) and slow (seconds to minutes) pathway and that the interplay between activation and inactivation pathways is held in a delicate balance.

The drugs are thought to stabilise an inactivated configuration of the channel that is adopted rapidly after the channel opens. This inactivated state provides a refractory period before the channel returns to its resting (closed) state ready to be reactivated. As a result, use-dependent sodium channel blockers retard the firing of neurons at high frequency, for example in response to painful stimuli, and will help to prevent repetitive firing during periods of prolonged neuronal depolarization that might occur, for example, during a seizure. Action potentials triggered at low frequencies, for example in the heart, will not be significantly affected by these drugs, although the safety margin differs in each case, since at high enough concentrations each of these drugs is capable of blocking the resting or open states of the channels.

The voltage-gated sodium channel family is made up of 10 subtypes, four of which are brain specific, NaV1.1, 1.2, 1.3 and 1.6. Of the other subtypes, NaV1.4 is found only in skeletal muscle, NaV1.5 is specific to cardiac muscle, and NaV1.7, 1.8, and 1.9 are found predominantly in sensory neurons. The hypothesized binding site for use-dependent sodium channel blockers is highly conserved between all the subtypes. As a result, drugs such as lidocaine, lamotrigine and carbamazepine do not distinguish between the subtypes. However, selectivity can be achieved as a result of the different frequencies at which the channels normally operate.

In general, drugs that interact with sodium channels to block ion flux cause the channels to inactivate to a greater extent and with smaller depolarizations than normal. Other sodium channel blockers, such as lamotrigine and carbamazepine are used to treat epilepsy. In the latter case, partial inhibition of voltage-gated sodium channels reduces neuronal excitability and reduces seizure propagation. In the case of local anesthetics, regional block of sodium channels on sensory neurons prevents the conduction of painful stimuli.

Drugs that block voltage-gated sodium channels in a use-dependent manner are also used in the treatment of bipolar disorder, either to reduce symptoms of mania or depression, or as mood stabilisers to prevent the emergence of mood episodes. Clinical and preclinical evidence also suggests that use-dependent sodium channel blockers may help to reduce the symptoms of schizophrenia. For example, lamotrigine has been shown to reduce symptoms of psychosis induced by ketamine in healthy human volunteers, and furthermore, studies in patients suggest that the drug can augment the antipsychotic efficacy of some atypical antipsychotic drugs, such as clozapine or olanzapine. It is hypothesized that efficacy in these psychiatric disorders may result in part from a reduction of excessive glutamate release. The reduction in glutamate release is thought to be a consequence of use-dependent sodium channel inhibition in key brain areas, such as the frontal cortex. However, interaction with voltage-gated calcium channels may also contribute to the efficacy of these drugs.

Propagation of nerve impulses arising from tussive stimuli is mediated, at least in part, via voltage-gated Na⁺ channels (NaV). Generation of the action potential is blocked by local anesthetics such as Lidocaine. Drugs, such as lidocaine, that block voltage-gated sodium channels are used as local anaesthetics.

Lidocaine reduces the inward sodium current which elicits neuronal impulses (BUTTERWORTH, J. F. T. & STRICHARTZ, G. R. (1990). Molecular mechanisms of local anesthesia: a review. Anesthesiology, 72, 711-34; MCCLEANE, G. (2007). Intravenous lidocaine: an outdated or underutilized treatment for pain? J Palliat Med, 10, 798-805). Common modes of drug action on Na⁺ channels: local anesthetics, antiarrhythmics and anticonvusants. TiPS, 8, 57-65; HILLE, B. (1966). Common mode of action of three agents that decrease the transient change in sodium permeability in nerves. Nature, 210, 1220-2; TAYLOR, R. E. (1959). Effect of procaine on electrical properties of squid axon membrane. Am J Physiol, 196, 1071-8.) Indeed, blockade of neuronal Na+ channels is one of the most powerful and well described analgesic principles (CATTERALL, W. A. & MACKIE, K. (2005). Chapter 14: Local Anesthetics. In Goodman & Gilman's The Pharmacological Basis of Therapeutics, 11th Edition. ed Brunton, L.) Lidocaine, a pan-NaV inhibitor, is used to minimize gagging and cough during bronchoconscopy (REED, A. P. (1992). Preparation of the patient for awake flexible fiberoptic bronchoscopy. Chest, 101, 244-53) and to limit airway intubation-induced post operative cough and sore throat (DIACHUN, C. A., TUNINK, B. P. & BROCK-UTNE, J. G. (2001). Suppression of cough during emergence from general anesthesia: laryngotracheal lidocaine through a modified endotracheal tube. J Clin Anesth, 13, 447-51).

In general, coughing is produced in a variety of airway diseases, which may enhance and intensify the cough response. The cough reflex protects the airway from potential harm by aiding the clearance of luminal debris. Within the airway epithelium, irritant sensing vagal nerve endings transmit information arising from the presence of tussive stimuli to the brain stem evoking an urge to cough. Chronic cough, often thought as dry and unproductive, is associated with progressive irreversible lung damage such as occurs in chronic obstructive pulmonary disease (COPD). The persistence and intensity of this form of cough robs patients of quality of life. It is this inappropriate chronic cough, a common symptom of chronic respiratory disease that therapy aims to resolve.

Based on the foregoing, there is evidence suggesting that short-term administration of intravenous lidocaine may produce pain relief that far exceeds both the duration of infusion and the half-life of the drug (McCleane, 2007). Although widely investigated, the mechanism remains unknown. One possibility is that local anesthetics inhibit central sensitization, i.e., the long-term increase in the excitability of the central nervous system in response to on-going or repeated activation of nociceptors. Blockade of sensory nerve input even for a short time would allow restoration of normal nerve function, a similar long-lasting effect on intractable dry cough could be expected.

In light of the above, a need exists to develop compounds of the present invention, corresponding compositions and treatment methods for diseases associated with mediation or modulation of voltage-gated sodium channels, which include, but are not limited to respiratory diseases or associated disorders

The present invention is directed to overcoming these and other problems encountered in the art.

### SUMMARY OF THE INVENTION

In general, the present invention relates to voltage-gated sodium channel blocker compounds, which include corresponding precursors, intermediates, monomers and dimers, compound preparation methods, pharmaceutical compositions and treatment methods directed to respiratory or respiratory tract diseases.

In particular, the present invention relates to novel compounds of Formulas (I) to (XVI) and corresponding pharmaceutical compositions comprising compounds of Formulas (I) to (XVI), respectively.

The present invention also relates to processes for making compounds of Formulas (I) to (XVI), respectively.

The present invention also relates to methods for the treatment of respiratory or respiratory tract diseases, which comprises administering to a subject in need thereof an effective amount of a compound of Formulas (I) to (XVI), respectively.

### DETAILED DESCRIPTION OF THE INVENTION

In general, the present invention relates to voltage-gated sodium channel blocker compounds, which include corresponding precursors, intermediates, monomers and dimers, compound preparation methods, pharmaceutical compositions and treatment methods directed to respiratory or respiratory tract diseases.

### Compounds

A. Precursors, Intermediates and Monomers

In particular, the present invention relates to novel compounds of Formulas (I) to (XVI) and corresponding pharmaceutical compositions comprising compounds of Formulas (I) to (XVI), respectively.

In one aspect, the present invention relates to a compound of Formula (I):

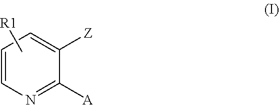

wherein:
R₁ is H, halogen, straight or branched C₁₋₆ alkyl, phenyl, substituted phenyl, —NHRₐ, —SRₐ or —ORₐ;

### A is

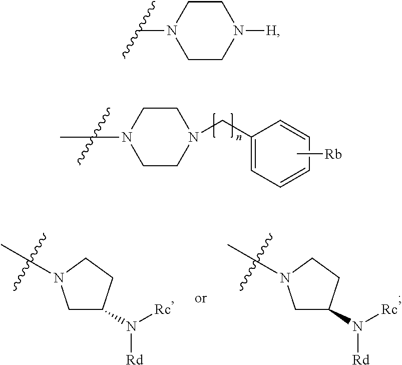

### Z is

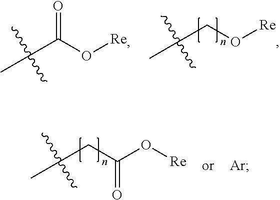

wherein:

n is 0 or an integer from 1 to 5;

halogen is selected from bromo, chloro, fluoro or iodo;

Rₐ is phenyl or substituted phenyl;

R_{b} is H, halogen, —C(O)H, —C(O)—OH, —C(O)—OR₁ₐ, —(CH)O(R_{1b})₂, —(CH₂)ₘN—R_{1c}, —NH₂, —NHC(O)-phenyl, —NHC(O)-substituted phenyl, —NO₂, —SH, or —SR_{1d};

R_{c} is H, straight or branched C₁₋₆ alkyl, cycloalkyl; phenyl or heteroaryl;

R_{d} is H, straight or branched C₁₋₆ alkyl or cycloalkyl;

Rₑ is H, straight or branched C₁₋₆ alkyl or cycloalkyl;

Ar is aryl or heteroaryl;
wherein:
   for each variable Rₐ, R_{b}, R_{c}, R_{d} or Rₑ:
      halogen as defined for Rb is bromo, chloro, fluoro or iodo;
   R₁ₐ, R_{1b}, R_{1c}, or R_{1d} is H or straight or branched C₁₋₆ alkyl; or
      a pharmaceutically acceptable salt thereof.

Representative compounds of Formula (I) may include, but is not limited to, the following compounds:
1-methylethyl 2-{(3R)-3-[{[(1,1-dimethylethyl)oxy]carbonyl}(ethyl)amino]-1-pyrrolidinyl}-4-iodo-3-pyridinecarboxylate;
1-methylethyl 2-{(3R)-3-[{[(1,1-dimethylethyl)oxy]carbonyl}(ethyl)amino]-1-pyrrolidinyl}-4-phenyl-3-pyridinecarboxylate;
1-methylethyl 2-[(3R)-3-(ethylamino)-1-pyrrolidinyl]-4-phenyl-3-pyridinecarboxylate;
(R)-Isopropyl 2-[3-(ter-butoxycarbonylamino)pyrrolidine-1-yl]nicotinate;
(R)-Isopropyl 2-{3-[tert-butoxycarbonyl(ethyl)amino]pyrrolidin-1-yl}nicotinate;
1-Methylethyl 2-[(3S)-3-({[(1,1-dimethylethyl)oxy]carbonyl}amino)-1-pyrrolidinyl]-3-pyridinecarboxylate;
1-Methylethyl 2-[(3S)-3-amino-1-pyrrolidinyl]-3-pyridinecarboxylate;
1-Methylethyl 2-{(3S)-3-[{[(1,1-dimethylethyl)oxy]carbonyl}(methyl)amino]-1-pyrrolidinyl}-3-pyridinecarboxylate;
1-Methylethyl 2-[(3S)-3-(methylamino)-1-pyrrolidinyl]-3-pyridinecarboxylate;
1-Methylethyl 2-{(3S)-3-[{[(1,1-dimethylethyl)oxy]carbonyl}(ethyl)amino]-1-pyrrolidinyl}-3-pyridinecarboxylate;
1-Methylethyl 2-[(3S)-3-(ethylamino)-1-pyrrolidinyl]-3-pyridinecarboxylate;
1,1-Dimethylethyl [(3R)-1-(2-methylpropanoyl)-3-pyrrolidinyl]carbamate;
1,1-Dimethylethyl ethyl[(3R)-1-(2-methylpropanoyl)-3-pyrrolidinyl]carbamate;
1-Methylethyl 2-[(3R)-3-({[(1,1-dimethylethyl)oxy]carbonyl}amino)-1-pyrrolidinyl]-3-pyridinecarboxylate;
1-Methylethyl 2-{(3R)-3-[{[(1,1-dimethylethyl)oxy]carbonyl}(ethyl)amino]-1-pyrrolidinyl}-3-pyridinecarboxylate;
1-methylethyl 4-iodo-2-[4-(phenylmethyl)-1-piperazinyl]-3-pyridine carboxylate;
1-methylethyl 2-(1-piperazinyl)-3-pyridinecarboxylate;
1-methylethyl 2-{4-[(2-bromophenyl)methyl]-1-piperazinyl}-3-pyridinecarboxylate;
1-methylethyl 2-{4-[(3-bromophenyl)methyl]-1-piperazinyl}-3-pyridinecarboxylate;
1-methylethyl 2-{4-[(4-bromophenyl)methyl]-1-piperazinyl}-3-pyridinecarboxylate;
1-methylethyl 4-phenyl-2-(1-piperazinyl)-3-pyridinecarboxylate;
1-methylethyl 4-methyl-2-(1-piperazinyl)-3-pyridinecarboxylate;
1-methylethyl 2-[4-({4-[(ethylamino)methyl]phenyl}methyl)-1-piperazinyl]-3-pyridinecarboxylate;
1-Methylethyl2-{4-[(4-mercaptophenyl)methyl]-1-piperazinyl}-3-pyridinecarboxylate;
1,1-Dimethylethyl4-(3-{[(1-methylethyl)oxy]carbonyl}-2-pyridinyl)-1-piperazinecarboxylate;
1-Methylethyl 2-(1-piperazinyl)-3-pyridinecarboxylate;
1-Methylethyl2-{4-([4-[bis(ethyloxy)methyl]phenyl}methyl)-1-piperazinyl]-3-pyridine carboxylate;
1-Methylethyl 2-{4-[(4-formylphenyl)methyl]-1-piperazinyl}-3-pyridinecarboxylate;
1-Methylethyl2-{4-[(3-nitrophenyl)methyl]-1-piperazinyl}-3-pyridinecarboxylate;
1-Methylethyl 2-{4-[(3-aminophenyl)methyl]-1-piperazinyl}-3-pyridinecarboxylate;
1-Methylethyl 2-[4-({3-[(phenylcarbonyl)amino]phenyl}methyl)-1-piperazinyl]-3-pyridinecarboxylate;
1-Methylethyl 2-{4-[(4-nitrophenyl)methyl]-1-piperazinyl}-3-pyridinecarboxylate;
1-Methylethyl 2-{4-[(4-aminophenyl)methyl]-1-piperazinyl}-3-pyridinecarboxylate;
1-Methylethyl 2-[4-({4-[(phenylcarbonyl)amino]phenyl}methyl)-1-piperazinyl]-3-pyridine carboxylate;
3-{[4-(3-{[(1-Methylethyl)oxy]carbonyl}-2-pyridinyl)-1-piperazinyl]methyl}benzoic acid;
4-{[4-(3-{[(1-Methylethyl)oxy]carbonyl}-2-pyridinyl)-1-piperazinyl]methyl}benzoic acid;
1-Methylethyl 2-{[((2S)-1-{[(1,1-dimethylethyl)oxy]carbonyl}-2-pyrrolidinyl)methyl]oxy}-3-pyridinecarboxylate;
1-Methylethyl 2-{[((2R)-1-{[(1,1-dimethylethyl)oxy]carbonyl}-2-pyrrolidinyl)methyl]oxy}-3-pyridinecarboxylate;
1,1-Dimethylethyl ethyl{(3R)-1-[3-(hydroxymethyl)-2-pyridinyl]-3-pyrrolidinyl}carbamate;
(2-{(3R)-3-[{[(1,1-Dimethylethyl)oxy]carbonyl}(ethyl)amino]-1-pyrrolidinyl}-3-pyridinyl)methyl benzoate;
{2-[(3R)-3-(Ethylamino)-1-pyrrolidinyl]-3-pyridinyl}methyl benzoate;
(2-{(3R)-3-[{[(1,1-Dimethylethyl)oxy]carbonyl}(ethyl)amino]-1-pyrrolidinyl}-3-pyridinyl)methyl 3,3-dimethylbutanoate;
(2-{(3R)-3-[{[(1,1-Dimethylethyl)oxy]carbonyl}(ethyl)amino]-1-pyrrolidinyl}-3-pyridinyl)methyl 3,3-dimethylbutanoate;
{2-[(3R)-3-(Ethylamino)-1-pyrrolidinyl]-3-pyridinyl}methyl 3,3-dimethylbutanoate;
1-Methylethyl 2-{[(2S)-2-pyrrolidinylmethyl]oxy}-3-pyridinecarboxylate;
1-Methylethyl 2-{[(2R)-2-pyrrolidinylmethyl]oxy}-3-pyridinecarboxylate; or
   a pharmaceutically acceptable salt thereof.

In another aspect, the present invention relates to a compound of Formula (II):

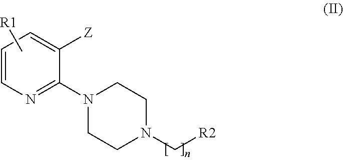

wherein:
n is 0 or an integer from 1 to 5;
R₁ is —H, -halogen,-straight or branched C₁₋₆ alkyl, -phenyl, -substituted phenyl, —NHRₐ, —SRₐ or —ORₐ;

wherein as defined for R₁:
halogen is bromo, chloro, fluoro or iodo;
Rₐ is -phenyl or -substituted phenyl;
   R₂ is aryl or heteroaryl;

wherein aryl is selected from -phenyl or -substituted phenyl;

wherein heteroaryl is selected from mono, bicyclic or tricyclic heterocyclic aromatic ring compounds containing 1-3 hetero atoms independently selected from nitrogen, oxygen and sulphur;
wherein aryl or heteroaryl further optionally is substituted by one or more substituents from Group A selected from:
—H, —OH, —CN, halogen, straight or branched C₁₋₆ alkyl, -straight or branched C₁₋₆ haloalkyl, -straight or branched C₁₋₆ alkoxy, aryl or heteroaryl, —O(CH₂)ₓOR₁ₐ, —C(O)R_{1b}, —C(O)OR_{1c}, aryl or heteroaryl, —(CH₂)ₓ-aryl, —(CH₂)ₓ-substituted aryl, —(CH₂)ₓ-heteroaryl, —(CH₂)ₓ-substituted heteroaryl, —O—(CH₂)ₓ-aryl, —O—(CH₂)ₓ-substituted aryl, —O—(CH₂)ₓ-heteroaryl, —O—(CH₂)ₓ-substituted heteroaryl, S-aryl, —S(CH₂)ₓ aryl, —S(CH₂)ₓ substituted aryl, S-heteroaryl, —S(CH₂)ₓ heteroaryl, —S(CH₂)ₓ substituted heteroaryl; NH-aryl, —NR(CH₂)ₓ aryl, —NR(CH₂)ₓ substituted aryl, NR-heteroaryl, —NR(CH₂)ₓ heteroaryl, —NR(CH₂)ₓ substituted heteroaryl, —(CH₂)ₓ—N(R_{1d})—(CH₂)ₓR₁ₑ;
wherein:
   R₁ₐ, R_{1b}, R_{1c}, or R_{1d} as defined in R₂ above is H or straight or branched C₁₋₆ alkyl;
   R₁ₑ is H or straight or branched C₁₋₆ alkyl, phenyl, substituted phenyl, furanyl, substituted furanyl, thienyl, or substituted thienyl;
   x as defined for substituents defined above is 0 or an integer from 1 to 5,
   wherein:
   each substitutent as defined in Group A above further is optionally substituted by one or more of following substituents selected from: —H, —OH, —CN, —NO₂, -halogen, —(CH₂)_{y}—OH, —O(CH₂)_{y} CN, —OC(O)OH, —OC(O)R_{1f}, —C(O)OR_{1g}, —O(CH₂)_{y}OR₁ₕ, -straight or branched C₁₋₆ alkyl, -straight or branched C₁₋₆ haloalkyl, -straight or branched C₁₋₆ straight or branched alkoxy, —NR₁ᵢR₁ⱼ, —SO₂R₁ₖ, —S(CH₂)_{y}R₁ₗ, —NR₁ₘC(O)R₁ₙ, aryl or heteroaryl;
      wherein:
      y as defined for variables defined for Group A above is 0 or an integer from 1 to 5,
      R_{1f}, R_{1g}, R₁ₕ, R₁ᵢ, R₁ⱼ, R₁ₖ, R₁ₗ, R₁ₘ or R₁ₙ is H or straight or branched C₁₋₆ alkyl;

### Z is

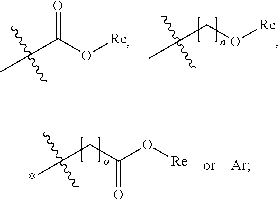

wherein:
Rₑ is H or straight or branched C₁₋₆ alkyl or cycloalkyl;
Ar is aryl;
n is 0 or an integer from 1 to 5;
o is 0 or an integer from 1 to 5; or
   a pharmaceutically acceptable salt thereof.

In another aspect, the present invention relates to a compound of Formula (III):

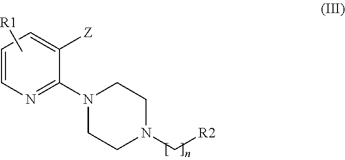

wherein:
n is 0 or an integer from 1 to 5;
R₁ is H, halogen, straight or branched C₁₋₆ alkyl, phenyl, substituted phenyl, —NHR₁ₐ, —SR_{1b}, or —OR_{1c};
R₂ is phenyl, substituted phenyl, —(CH₂)ₓ-phenyl, furanyl, —(CH₂)ₓ furanyl, -thienyl, —(CH₂)ₓ thienyl, (CH₂)ₓ thiazolyl, —(CH₂)ₓ pyrazolyl, —(CH₂)ₓ isoxazolyl, —(CH₂)ₓ pyrrolidinyl, —(CH₂)ₓ pyridinyl, —(CH₂)ₓ substituted pyridinyl, —(CH₂)ₓ pyrazinyl, —(CH₂)ₓ substituted pyrazinyl, -phenoxy, —(CH₂)ₓ-phenoxy, —(CH₂)ₓ-substituted phenoxy, —(CH₂)ₓ-substituted phenoxy, —(CH₂)ₓ-dibenzofuranyl, —(CH₂)ₓ-substituted dibenzofuranyl, —(CH₂)ₓ-carbazolyl, —(CH₂)ₓ-substituted carbazolyl, —(CH₂)ₓ-1,2,3,4 tetrahydro isoquinolinyl, —(CH₂)ₓ-substituted 1,2,3,4 tetrahydro isoquinolinyl, —(CH₂)ₓ-fluorenyl, or —(CH₂)ₓ-substituted fluorenyl;

wherein:
R₁ₐ, R_{1b} or R_{1c} as defined for R₁ is phenyl or substituted phenyl;
x as defined for substituents defined above is 0 or an integer from 1 to 5,
R₂ further optionally is substituted with at least one or more substituents selected from Group A:
—H, —OH, —CN, halogen, straight or branched C₁₋₆ alkyl, -straight or branched C₁₋₆ haloalkyl, -straight or branched C₁₋₆ alkoxy, aryl or heteroaryl,—O(CH₂)_{y}OR_{1d}, —C(O)R₁ₑ, —C(O)OR_{1f}, —(CH₂)_{y}—N(R_{1g}) —(CH₂)_{y}R₁ₕ, aryl or heteroaryl, —(CH₂)_{y}-aryl, —(CH₂)_{y}-substituted aryl, —(CH₂)_{y}-heteroaryl, —(CH₂)_{y}-substituted heteroaryl, —O—(CH₂)_{y}-aryl, —O—(CH₂)_{y}-substituted aryl, —O—(CH₂)_{y}-heteroaryl, —O—(CH₂)_{y}-substituted heteroaryl, S-aryl, —S(CH₂)_{y} aryl, —S(CH₂)_{y} substituted aryl, S-heteroaryl, —S(CH₂)_{y} heteroaryl, —S(CH₂)_{y} substituted heteroaryl; NH-aryl, —NR(CH₂)_{y} aryl, —NR(CH₂)_{y}substituted aryl, NR-heteroaryl, —NR(CH₂)_{y} heteroaryl, —NR(CH₂)_{y} substituted heteroaryl, —(CH₂)_{y}—N(R_{1g})—(CH₂)_{z}R₁ₕ,
wherein:
   R_{1d}, R₁ₑ, R_{1f}, or R_{1g} as defined in R₂ is H or straight or branched C₁₋₆ alkyl;
   R₁ₕ is phenyl, substituted phenyl, furanyl, substituted furanyl, thienyl, or substituted thienyl;
   y as defined for substituents defined above is 0 or an integer from 1 to 5,
   wherein:
   each substitutent as defined in Group A above further is optionally substituted by one or more of following substituents selected from: —H, —OH, —CN, —NO₂,-halogen, —(CH₂)_{z}—OH, —O(CH₂)_{z} CN, —OC(O)OH, —OC(O)R₁ᵢ, —C(O)OR₁ⱼ, —O(CH₂)_{z}OR₁ₖ,-straight or branched C₁₋₆ alkyl,-straight or branched C₁₋₆ haloalkyl, -straight or branched C₁₋₆ straight or branched alkoxy, —NR₁ₗR₁ₘ, —SO₂R₁ₙ, —S(CHA₂)_{z}R₁ₒ, —NR₁ₚC(O)R_{1g}, aryl or heteroaryl;
      wherein:
      z as defined for variables above is 0 or an integer from 1 to 5;
      R₁ᵢ, R₁ⱼ, R₁ₖ, R₁ₗ, R₁ₘ, R₁ₙ, R₁ₒ, R₁ₚ or R_{1q} is H or straight or branched C₁₋₆ alkyl;

### Z is

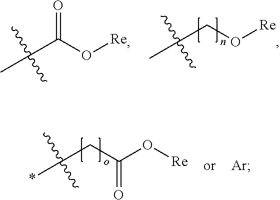

wherein:
n is 0 or an integer from 1 to 5;
o is 0 or an integer from 1 to 5;
Rₑ is H, straight or branched C₁₋₆ alkyl or cycloalkyl;
Ar is aryl or heteroaryl; or
   a pharmaceutically acceptable salt thereof.

Representative compounds of Formula (III), may include, but are not limited to:
1-methylethyl 2-{4-[(5-{[[(2-chloro-6-fluorophenyl)methyl](ethyl)amino]methyl}-2-pyridinyl)methyl]-1-piperazinyl}-3-pyridinecarboxylate;
1-methylethyl2-{4-[(6-{[[(2-chloro-6-fluorophenyl)methyl](ethyl)amino]methyl}-3-pyridinyl)methyl]-1-piperazinyl}-3-pyridinecarboxylate; 1-methylethyl2-{4-[(6-{[[(2-chloro-6-fluorophenyl)methyl](ethyl)amino]methyl}-3-pyridinyl)methyl]-1-piperazinyl}-3-pyridinecarboxylate trihydrochloride;
1-methylethyl2-[4-({2-[(2-chloro-6-fluorophenyl)methyl]-1,2,3,4-tetrahydro-6-isoquinolinyl}methyl)-1-piperazinyl]-3-pyridinecarboxylate; (2-{4-[(4-{[[(2-chloro-6-fluorophenyl)methyl](ethyl)amino]methyl}phenyl)methyl]-1-piperazinyl}-3-pyridinyl)methyl 2,2,3,3-tetramethylcyclopropanecarboxylate; (2-{4-[(4-{[[(2-chloro-6-fluorophenyl)methyl](ethyl)amino]methyl}phenyl)methyl]-1-piperazinyl}-3-pyridinyl)methyl 3,3-dimethylbutanoate;
(2-{4-[(4-{[[(2-chloro-6-fluorophenyl)methyl](ethyl)amino]methyl}phenyl)methyl]-1-piperazinyl}-3-pyridinyl)methyl 2-methylpropanoate; (2-{4-[(4-{[[(2-chloro-6-fluorophenyl)methyl](ethyl)amino]methyl}phenyl)methyl]-1-piperazinyl}-3-pyridinyl)methyl acetate; 1-methylethyl2-{4-[(5-{[[(2-chloro-6-fluorophenyl)methyl](ethyl)amino]methyl}-2-pyrazinyl)methyl]-1-piperazinyl}-3-pyridinecarboxylate;
(2-{4-[(4-{[[(2-chloro-6-fluorophenyl)methyl](ethyl)amino]methyl}phenyl)methyl]-1-piperazinyl}-3-pyridinyl)methyl cyclopropanecarboxylate; (2-{4-[(4-{[[(2-chloro-6-fluorophenyl)methyl](ethyl)amino]methyl}phenyl)methyl]-1-piperazinyl}-3-pyridinyl)methyl propanoate; or
   a pharmaceutically acceptable salt thereof.

In another aspect, the present invention relates to a compound of Formula (IV):

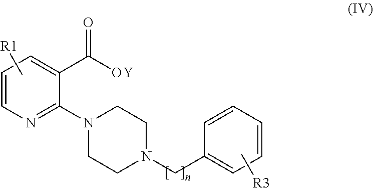

wherein:
n is 0 or an integer from 1 to 5;
Y is straight or branched C₁₋₆ alkyl or cycloalkyl;
R₁ is H, halogen, straight or branched C₁₋₆ alkyl, phenyl, substituted phenyl, —NHR₁ₐ, —SR_{1b} or —OR_{1c};
R₃ is one or more substituents independently selected from —H, —OH, —CN, halogen, straight or branched C₁₋₆ alkyl, -straight or branched C₁₋₆ haloalkyl, -straight or branched C₁₋₆ alkoxy, -straight or branched C₁₋₆ alkoxy, —O(CH₂)ₓOR_{1d}, —C(O)R₁ₑ, —C(O)OR_{1f}, -phenyl, —(CH₂)ₓ-phenyl, —(CH₂)ₓ-substituted phenyl, -phenyloxy, -substituted phenyloxy, —(CH₂)ₓ-phenyloxy, —(CH₂)ₓ-piperazinyl, —(CH₂)ₓ-substituted piperazinyl, -(CH₂)ₓ—N-substituted piperazinyl, —(CH₂)), NRC(O)-phenyl, —(CH₂)ₓ NRC(O)-substituted phenyl, —O—(CH₂)ₓ-phenyl, —O—(CH₂)ₓ-substituted phenyl, —O(CH₂)ₓ-1,4-benzodioxinyl, —O(CH₂)ₓ-naphthalenyl, —O(CH₂)ₓ-tetrazolyl, —S-phenyl, —S(CH₂)ₓ phenyl, —SO₂R_{1g}, —SO₂N(R_{1g})₂, —(CH₂)ₓ—N(R₁ₙ)—(CH₂)ₓ

wherein:
R₁ₐ, R_{1b} or R_{1c} as defined in R₁ above is phenyl or substituted phenyl;
R, R_{1d}, R₁ₑ, R_{1f}, R_{1g} or R₁ₕ as defined in R₃ is H, straight or branched C₁₋₆ alkyl;
R₁ᵢ is phenyl, substituted phenyl, furanyl, substituted furanyl, thienyl, or substituted thienyl;
x as defined for substituents defined above is 0 or an integer from 1 to 5;
   wherein:
   each substitutent as defined in R₃ above further is optionally substituted by one or more of following substituents selected from: —H, —OH, —CN, —NO₂,-halogen, —(CH₂)_{y}—OH, —O(CH₂)_{y} CN, —OC(O)OH, —OC(O)R₁ⱼ, —C(O)OR₁ₖ, —O(CH₂)_{y}OR₁ₗ,-straight or branched C₁₋₆alkyl,-straight or branched C₁₋₆ haloalkyl, -straight or branched C₁₋₆ alkoxy, NR₁ₘR₁ₙ, —SO₂R₁ₒ, —S(CH₂)_{y}R₁ₚ, —NR_{1q},C(O)R₁ᵣ, aryl or heteroaryl;
      wherein:
      y as defined for variables above is 0 or an integer from 1 to 5,
      R₁ⱼ, R₁ₖ, R₁ₗ, R₁ₘ, R₁ₙ, R₁ₒ, R₁ₚ, R_{1q}, or R₁ᵣ, is H, straight or branched C₁₋₆ alkyl, phenyl, substituted phenyl, pyridinyl, or substituted pyridinyl, —C(O)-phenyl, —C(O)substituted phenyl or (CH₂)ₓ-2-oxo-1-pyrrolidinyl or (CH₂)ₓ-2-oxo-N-pyrrolidinyl; or
         wherein:
         x is 0 or an integer from 1 to 5;
         each phenyl or substituted phenyl substitutent as defined in R₁ⱼ, R₁ₖ, R₁ₗ, R₁ₘ, R₁ₘ, R₁ₒ, R₁ₚ, R_{1q} or R₁ᵣ above further is optionally substituted by one or more of following substituents selected from: —H, —OH, —CN, —NO₂,-halogen, —(CH₂)_{y}—OH, —OC(O)OH, —OC(O)R₁ₛ, —C(O)OR₁ₜ, —SO₂N(R₁ᵤ)₂—, straight or branched C₁₋₆ alkyl,-straight or branched C₁₋₆ haloalkyl, -straight or branched C₁₋₆ alkoxy;
         wherein:
         R₁ₛ, R₁ₜ, or R₁ᵤ as defined above is H, straight or branched C₁₋₆ alkyl, phenyl or substituted phenyl; or
            a pharmaceutically acceptable salt thereof.

Representative compounds of Formula (IV), may include, but are not limited to:
1-methylethyl 2-[4-({3-[(2-thienylmethyl)oxy]phenyl}methyl)-1-piperazinyl]-3-pyridinecarboxylate;
1-methylethyl 2-{4-[(3-{[(2,6-dichlorophenyl)methyl]oxy}phenyl)methyl]-1-piperazinyl}-3-pyridinecarboxylate;
1-methylethyl 2-{4-[(3-{[(3-chlorophenyl)methyl]oxy}phenyl)methyl]-1-piperazinyl}-3-pyridinecarboxylate;
1-methylethyl 2-{4-[(3-{[(2-chloro-4-fluorophenyl)methyl]oxy}phenyl)methyl]-1-piperazinyl}-3-pyridinecarboxylate;
1-methylethyl 2-{4-[(3-{[(4-methylphenyl)methyl]oxy}phenyl)methyl]-1-piperazinyl}-3-pyridinecarboxylate;
1-methylethyl 2-{4-[(3-{[(2-methylphenyl)methyl]oxy}phenyl)methyl]-1-piperazinyl}-3-pyridinecarboxylate;
1-methylethyl 2-{4-[(3-{[(3-fluorophenyl)methyl]oxy}phenyl)methyl]-1-piperazinyl}-3-pyridinecarboxylate;
1-methylethyl 2-{4-[(3-{[(4-nitrophenyl)methyl]oxy}phenyl)methyl]-1-piperazinyl}-3-pyridinecarboxylate;
1-methylethyl 2-{4-[(3-{[(2-chlorophenyl)methyl]oxy}phenyl)methyl]-1-piperazinyl}-3-pyridinecarboxylate;
1-methylethyl 2-(4-{[3-({[3-(trifluoromethyl)phenyl]methyl}oxy)phenyl]methyl}-1-piperazinyl)-3-pyridinecarboxylate;
1-methylethyl 2-{4-[(3-{[(2,4-dichlorophenyl)methyl]oxy}phenyl)methyl]-1-piperazinyl}-3-pyridinecarboxylate;
1-methylethyl 2-{4-[(3-{[(3-methylphenyl)methyl]oxy}phenyl)methyl]-1-piperazinyl}-3-pyridinecarboxylate;
1-methylethyl 2-(4-{[3-(ethyloxy)phenyl]methyl}-1-piperazinyl)-3-pyridine carboxylate;
1-methylethyl 2-{4-[(3-{[(2-chloro-6-fluorophenyl)methyl]oxy}phenyl)methyl]-1-piperazinyl}-3-pyridinecarboxylate;
1-methylethyl 2-(4-{[3-(acetyloxy)phenyl]methyl}-1-piperazinyl)-3-pyridine carboxylate;
1-methylethyl 2-[4-({3-[(1,1,2,2-tetrafluoroethyl)oxy]phenyl}methyl)-1-piperazinyl]-3-pyridinecarboxylate;
1-methylethyl 2-[4-({3[(2-methylpropyl)oxy]phenyl}methyl)-1-piperazinyl]-3-pyridinecarboxylate;
1-methylethyl 2-(4-{[3-(propyloxy)phenyl]methyl}-1-piperazinyl)-3-pyridine carboxylate;
[(3-{[4-(3-{[(1-methylethyl)oxy]carbonyl}-2-pyridinyl)-1-piperazinyl]methyl}phenyl) oxy]acetic acid;
1-methylethyl 2-[4-({3-[(2-hydroxyethyl)oxy]phenyl}methyl)-1-piperazinyl]-3-pyridinecarboxylate;
1-methylethyl 2-[4-({3-[(phenylmethyl)oxy]phenyl}methyl)-1-piperazinyl]-3-pyridine carboxylate;
1-methylethyl 2-(4-{[3-({2-[(2-chloroethyl)oxy]ethyl}oxy)phenyl]methyl}-1-piperazinyl)-3-pyridinecarboxylate;
1-methylethyl 2-{4-[(3-{[(4-fluorophenyl)methyl]oxy}phenyl)methyl]-1-piperazinyl}-3-pyridinecarboxylate;
1-methylethyl 2-{4-[(3-{[(4-chlorophenyl)methyl]oxy}phenyl)methyl]-1-piperazinyl}-3-pyridinecarboxylate;
1-methylethyl 2-[4-(phenylmethyl)-1-piperazinyl]-4-(phenyloxy)-3-pyridine carboxylate;
1-methylethyl 4-[(2-fluorophenyl)amino]-2-[4-(phenylmethyl)-1-piperazinyl]-3-pyridinecarboxylate;
1-methylethyl 4-[(3-chlorophenyl)amino]-2-[4-(phenylmethyl)-1-piperazinyl]-3-pyridinecarboxylate;
1-methylethyl 4-[(4-cyanophenyl)amino]-2-[4-(phenylmethyl)-1-piperazinyl]-3-pyridinecarboxylate;
1-methylethyl 4-{[2-(ethyloxy)phenyl]amino}-2-[4-(phenylmethyl)-1-piperazinyl]-3-pyridinecarboxylate;
1-methylethyl 4-{[4-(1-methylethyl)phenyl]amino}-2-[4-(phenylmethyl)-1-piperazinyl]-3-pyridinecarboxylate;
1-methylethyl 4-{[2-(1-methylethyl)phenyl]amino}-2-[4-(phenylmethyl)-1-piperazinyl]-3-pyridinecarboxylate;
1-methylethyl 4-({3-[(ethyloxy)carbonyl]phenyl}amino)-2-[4-(phenylmethyl)-1-piperazinyl]-3-pyridinecarboxylate;
1-methylethyl 4-[(2-ethylphenyl)amino]-2-[4-(phenylmethyl)-1-piperazinyl]-3-pyridinecarboxylate;
1-methylethyl 4-{[4-(methyloxy)phenyl]amino}-2-[4-(phenylmethyl)-1-piperazinyl]-3-pyridinecarboxylate;
1-methylethyl 4-(phenylamino)-2-[4-(phenylmethyl)-1-piperazinyl]-3-pyridine carboxylate;
1-methylethyl 2-[4-(phenylmethyl)-1-piperazinyl]-4-(phenylthio)-3-pyridine carboxylate;
1-methylethyl 4-{[2-(methyloxy)phenyl]thio}-2-[4-(phenylmethyl)-1-piperazinyl]-3-pyridinecarboxylate;
1-methylethyl 2-[4-({2-[(2-chlorophenyl)amino]phenyl}methyl)-1-piperazinyl]-3-pyridinecarboxylate;
1-methylethyl 2-{4-[(3-{[2-(trifluoromethyl)phenyl]amino}phenyl)methyl]-1-piperazinyl}-3-pyridinecarboxylate;
1-methylethyl 2-{4-[(3-{[2-(methyloxy)phenyl]amino}phenyl)methyl]-1-piperazinyl}-3-pyridinecarboxylate;
1-methylethyl 2-[4-({3-[(2-methylphenyl)amino]phenyl}methyl)-1-piperazinyl]-3-pyridinecarboxylate;
1-methylethyl 2-[4-({3-[(2,6-difluorophenyl)amino]phenyl}methyl)-1-piperazinyl]-3-pyridinecarboxylate;
1-methylethyl 2-[4-({3-[(2-fluorophenyl)amino]phenyl}methyl)-1-piperazinyl]-3-pyridinecarboxylate;
1-methylethyl 2-[4-({3-[(2-chlorophenyl)amino]phenyl}methyl)-1-piperazinyl]-3-pyridinecarboxylate;
1-methylethyl 2-(4-{[4-({2-[(trifluoromethyl)oxy]phenyl}amino)phenyl]methyl}-1-piperazinyl)-3-pyridinecarboxylate;
1-methylethyl 2-(4-{[4-({3-(ethyloxy)carbonyl]phenyl}amino)phenyl)methyl}-1-piperazinyl)-3-pyridinecarboxylate;
1-methylethyl 2-{4-[(4-{[2-fluoro-6-(trifluoromethyl)phenyl]amino}phenyl)methyl]-1-piperazinyl}-3-pyridinecarboxylate;
1-methylethyl 2-[4-({4-[(2,6-difluorophenyl)amino]phenyl}methyl)-1-piperazinyl]-3-pyridinecarboxylate;
1-methylethyl 2-[4-({4-[(2-fluorophenyl)amino]phenyl}methyl)-1-piperazinyl]-3-pyridinecarboxylate;
1-methylethyl 2-[4-({4-[(2-chlorophenyl)amino]phenyl}methyl)-1-piperazinyl]-3-pyridinecarboxylate;
1-methylethyl 2-{4-[(4-{[4-(methyloxy)phenyl]amino}phenyl)methyl]-1-piperazinyl}-3-pyridinecarboxylate;
1-methylethyl 2-[4-(2-furanylmethyl)-1-piperazinyl]-4-phenyl-3-pyridinecarboxylate;
1-methylethyl 2-(4-{[2-(ethyloxy)phenyl]methyl}-1-piperazinyl)-4-phenyl-3-pyridine carboxylate;
1-methylethyl 4-phenyl-2-[4-(2-thienylmethyl)-1-piperazinyl]-3-pyridinecarboxylate;
1-methylethyl 2-[4-(3-furanylmethyl)-1-piperazinyl]-4-phenyl-3-pyridinecarboxylate;
1-methylethyl 2-{4-[(5-methyl-2-thienyl)methyl]-1-piperazinyl}-4-phenyl-3-pyridine carboxylate;
1-methylethyl 4-phenyl-2-(4-{[3-(phenyloxy)phenyl]methyl}-1-piperazinyl)-3-pyridine carboxylate;
1-methylethyl 4-phenyl-2-(4-{[3-(phenyloxy)phenyl]methyl}-1-piperazinyl)-3-pyridine carboxylate;
1-methylethyl 4-phenyl-2-[4-({3-[(phenylmethyl)oxy]phenyl}methyl)-1-piperazinyl]-3-pyridinecarboxylate;
1-methylethyl 4-phenyl-2-[4-({3-[(phenylmethyl)oxy]phenyl}methyl)-1-piperazinyl]-3-pyridinecarboxylate;
1-methylethyl 2-[4-({3-(methyloxy)-4-[(phenylmethyl)oxy]phenyl}methyl)-1-piperazinyl]-4-phenyl-3-pyridinecarboxylate;
1-methylethyl 2-[4-({3-(methyloxy)-4-[(phenylmethyl)oxy]phenyl}methyl)-1-piperazinyl]-4-phenyl-3-pyridinecarboxylate;
1-methylethyl 2-{4-[(2-cyanophenyl)methyl]-1-piperazinyl}-4-phenyl-3-pyridine carboxylate;
1-methylethyl 4-phenyl-2-[4-({4-[(trifluoromethyl)oxy]phenyl}methyl)-1-piperazinyl]-3-pyridinecarboxylate;
1-methylethyl 4-phenyl-2-(4-{[4-(propyloxy)phenyl]methyl}-1-piperazinyl)-3-pyridinecarboxylate;
1-methylethyl 2-{4-[(2-methylphenyl)methyl]-1-piperazinyl}-4-phenyl-3-pyridine carboxylate;
1-methylethyl 4-phenyl-2-[4-({2-[(phenylmethyl)oxy]phenyl}methyl)-1-piperazinyl]-3-pyridinecarboxylate;
1-methylethyl 2-[4-({4-(methyloxy)-3-[(phenylmethyl)oxy]phenyl}methyl)-1-piperazinyl]-4-phenyl-3-pyridinecarboxylate;
1-methylethyl 2-[4-(2-biphenylylmethyl)-1-piperazinyl]-4-phenyl-3-pyridine carboxylate;
1-methylethyl 2-{4-[(3-fluoro-2-methylphenyl)methyl]-1-piperazinyl}-4-phenyl-3-pyridinecarboxylate;
1-methylethyl 2-[4-({2-[(1-methylethyl)oxy]phenyl}methyl)-1-piperazinyl]-4-phenyl-3-pyridinecarboxylate;
1-methylethyl 2-[4-({2-[(1-methylethyl)oxy]phenyl}methyl)-1-piperazinyl]-4-phenyl-3-pyridinecarboxylate;
1-methylethyl 2-{4-[(3-{[(2-chlorophenyl)methyl]oxy}phenyl)methyl]-1-piperazinyl}-4-phenyl-3-pyridinecarboxylate;
1-methylethyl 2-[4-({4-[(4-fluorophenyl)oxy]phenyl}methyl)-1-piperazinyl]-4-phenyl-3-pyridinecarboxylate;
1-methylethyl 2-(4-{[4-({[4-(ethyloxy)phenyl]oxy}methyl)phenyl]methyl}-1-piperazinyl)-3-pyridinecarboxylate;
1-methylethyl 2-(4-{[4-({[3-(methyloxy)phenyl]oxy}methyl)phenyl]methyl}-1-piperazinyl)-3-pyridinecarboxylate;
1-methylethyl 2-{4-[(4-{[(2,6-difluorophenyl)oxy]methyl}phenyl)methyl]-1-piperazinyl}-3-pyridinecarboxylate;
1-methylethyl 2-{4-[(4-{[(3,4-difluorophenyl)oxy]methyl}phenyl)methyl]-1-piperazinyl}-3-pyridinecarboxylate;
1-methylethyl 2-{4-[(4-{[(3-chloro-4-fluorophenyl)oxy]methyl}phenyl)methyl]-1-piperazinyl}-3-pyridinecarboxylate;
1-methylethyl 2-(4-{[4-({[4-(1,1-dimethylethyl)phenyl]oxy}methyl)phenyl]methyl}-1-piperazinyl)-3-pyridinecarboxylate;
1-methylethyl 2-(4-{[4-({[4-(methyloxy)phenyl]oxy}methyl)phenyl]methyl}-1-piperazinyl)-3-pyridinecarboxylate;
1-methylethyl 2-[4-({4-({3-[(trifluoromethyl)oxy]phenyl}oxy)methyl]phenyl}methyl)-1-piperazinyl)-3-pyridinecarboxylate;
1-methylethyl 2-(4-{[4-({[2,3-bis(methyloxy)phenyl]oxy}methyl)phenyl]methyl}-1-piperazinyl)-3-pyridinecarboxylate;
1-methylethyl 2-{4-[(4-{[(2-chlorophenyl)oxy]methyl}phenyl)methyl]-1-piperazinyl}-3-pyridinecarboxylate;
1-methylethyl 2-(4-{[4-({[3,5-bis(methyloxy)phenyl]oxy}methyl)phenyl]methyl}-1-piperazinyl)-3-pyridinecarboxylate;
1-methylethyl 2-(4-{[4-({[2-(trifluoromethyl)phenyl]oxy}methyl)phenyl]methyl}-1-piperazinyl)-3-pyridinecarboxylate;
1-methylethyl 2-{4-[(4-{[(3-cyanophenyl)oxy]methyl}phenyl)methyl]-1-piperazinyl}-3-pyridinecarboxylate;
1-methylethyl 2-{4-[(4-{[(2,4-dichlorophenyl)oxy]methyl}phenyl)methyl]-1-piperazinyl}-3-pyridinecarboxylate;
1-methylethyl 2-{4-[(4-{[(2-methylphenyl)oxy]methyl}phenyl)methyl]-1-piperazinyl}-3-pyridinecarboxylate;
1-methylethyl 2-{4-[(4-{[(4-methyl phenyl)oxy]methyl}phenyl)methyl]-1-piperazinyl}-3-pyridinecarboxylate;
1-methylethyl 2-{4-[(4-{[(4-fluorophenyl)oxy]methyl}phenyl)methyl]-1-piperazinyl}-3-pyridinecarboxylate;
1-methylethyl 2-{4-[(4-{[(4-cyanophenyl)oxy]methyl}phenyl)methyl]-1-piperazinyl}-3-pyridinecarboxylate;
1-methylethyl 2-(4-{[3-({[4-(ethyloxy)phenyl]oxy}methyl)phenyl]methyl}-1-piperazinyl)-3-pyridinecarboxylate;
1-methylethyl 2-(4-{[3-({[3-(methyloxy)phenyl]oxy}methyl)phenyl]methyl}-1-piperazinyl)-3-pyridinecarboxylate;
1-methylethyl 2-{4-[(3-{[(2,6-difluorophenyl)oxy]methyl}phenyl)methyl]-1-piperazinyl}-3-pyridinecarboxylate;
1-methylethyl 2-{4-[(3-{[(3,4-difluorophenyl)oxy]methyl}phenyl)methyl]-1-piperazinyl}-3-pyridinecarboxylate;
1-methylethyl 2-{4-[(3-{[(3-chloro-4-fluorophenyl)oxy]methyl}phenyl)methyl]-1-piperazinyl}-3-pyridinecarboxylate;
1-methylethyl 2-(4-{[3-({[4-(1,1-dimethylethyl)phenyl]oxy}methyl)phenyl]methyl}-1-piperazinyl)-3-pyridinecarboxylate;
1-methylethyl 2-(4-{[3-({[4-(methyloxy)phenyl]oxy}methyl)phenyl]methyl}-1-piperazinyl)-3-pyridinecarboxylate;
1-methylethyl 2-(4-{[3-({[2,3-bis(methyloxy)phenyl]oxy}methyl)phenyl]methyl}-1-piperazinyl)-3-pyridinecarboxylate;
1-methylethyl 2-{4-[(3-{[(2-chlorophenyl)oxy]methyl}phenyl)methyl]-1-piperazinyl}-3-pyridinecarboxylate;
1-methylethyl 2-(4-{[3-({[3,5-bis(methyloxy)phenyl]oxy}methyl)phenyl]methyl}-1-piperazinyl)-3-pyridinecarboxylate;
1-methylethyl 2-(4-{[3-({[2-(trifluoromethyl)phenyl]oxy}methyl)phenyl]methyl}-1-piperazinyl)-3-pyridinecarboxylate;
1-methylethyl 2-{4-[(3-{[(3-cyanophenyl)oxy]methyl}phenyl)methyl]-1-piperazinyl}-3-pyridinecarboxylate;
1-methylethyl 2-{4-[(3-{[(2,4-dichlorophenyl)oxy]methyl}phenyl)methyl]-1-piperazinyl}-3-pyridinecarboxylate;
1-methylethyl 2-{4-[(3-{[(4-methylphenyl)oxy]methyl}phenyl)methyl]-1-piperazinyl}-3-pyridinecarboxylate;
1-methylethyl 2-{4-[(3-{[(4-fluorophenyl)oxy]methyl}phenyl)methyl]-1-piperazinyl}-3-pyridinecarboxylate;
1-methylethyl 2-(4-{[3-({[2-(ethyloxy)phenyl]oxy}methyl)phenyl]methyl}-1-piperazinyl)-3-pyridinecarboxylate;
1-methylethyl 2-{4-[(3-{[(4-cyanophenyl)oxy]methyl}phenyl)methyl]-1-piperazinyl}-3-pyridinecarboxylate;
1-methylethyl 2-{4-[(4-{[ethyl(3-furanylmethyl)amino]methyl}phenyl)methyl]-1-piperazinyl}-3-pyridinecarboxylate;
1-methylethyl 2-[4-({4-[(ethyl{[3-(ethyloxy)phenyl]methyl}amino}methyl]phenyl]methyl)-1-piperazinyl)-3-pyridinecarboxylate;
1-methylethyl 2-(4-{[4-(ethyl{[(5-methyl-2-thienyl)methyl]amino}methyl)phenyl]methyl)}-1-piperazinyl)-3-pyridinecarboxylate;
1-methylethyl 2-{4-[(4-{[[(2-chloro-6-fluorophenyl)methyl](ethyl)amino]methyl}phenyl)methyl]-1-piperazinyl}-3-pyridinecarboxylate;
1-methylethyl 2-[4-({4-[(ethyl{[2-(ethyloxy)phenyl]methyl}amino)methyl]phenyl}methyl)-1-piperazinyl]-3-pyridinecarboxylate;
1-methylethyl 2-[4-({4-[(ethyl{[3-(methyloxy)phenyl]methyl}amino)methyl]phenyl}methyl)-1-piperazinyl]-3-pyridinecarboxylate;
1-methylethyl 2-{4-[(4-{[ethyl(2-furanylmethyl)amino]methyl}phenyl)methyl]-1-piperazinyl}-3-pyridinecarboxylate;
1-methylethyl 2-{4-[(4-{[ethyl(2-thienylmethyl)amino]methyl}phenyl)methyl]-1-piperazinyl}-3-pyridinecarboxylate;
1-methylethyl 4-methyl-2-[4-({4-[(methyloxy)carbonyl]phenyl}methyl)-1-piperazinyl]-3-pyridinecarboxylate;
1-methylethyl 4-methyl-2-[4-(phenylmethyl)-1-piperazinyl]-3-pyridinecarboxylate;
1-methylethyl 4-methyl-2-(4-{[4-(methyloxy)phenyl]methyl}-1-piperazinyl)-3-pyridinecarboxylate;
1-methylethyl 2-{4-[(2-cyanophenyl)methyl]-1-piperazinyl}-4-methyl-3-pyridine carboxylate;
1-methylethyl 2-[4-(2-furanylmethyl)-1-piperazinyl]-4-methyl-3-pyridinecarboxylate;
1-methylethyl 2-{4-[(3-fluorophenyl)methyl]-1-piperazinyl}-4-methyl-3-pyridine carboxylate;
1-methylethyl 4-methyl-2-(4-{[3-(methyloxy)phenyl]methyl}-1-piperazinyl)-3-pyridinecarboxylate;
1-methylethyl 2-[4-(3-furanylmethyl)-1-piperazinyl]-4-methyl-3-pyridinecarboxylate;
1-methylethyl 4-methyl-2-{4-[(5-methyl-2-thienyl)methyl]-1-piperazinyl}-3-pyridine carboxylate;
1-methylethyl 2-{4-[(4-cyanophenyl)methyl]-1-piperazinyl}-4-methyl-3-pyridine carboxylate;
1-methylethyl 2-{4-[(3-cyanophenyl)methyl]-1-piperazinyl}-4-methyl-3-pyridine carboxylate;
1-methylethyl 2-{4-[(3-cyano-4-fluorophenyl)methyl]-1-piperazinyl}-4-methyl-3-pyridinecarboxylate;
1-methylethyl 2-{4-[(1,3-dimethyl-1H-pyrazol-4-yl)methyl]-1-piperazinyl}-4-methyl-3-pyridinecarboxylate;
1-methylethyl 2-{4-[(3,5-dimethyl-4-isoxazolyl)methyl]-1-piperazinyl}-4-methyl-3-pyridinecarboxylate;
1-methylethyl 2-(4-{[4-(acetylamino)phenyl]methyl}-1-piperazinyl)-4-methyl-3-pyridinecarboxylate;
1-methylethyl 2-(4-{[4-(acetyloxy)phenyl]methyl}-1-piperazinyl)-4-methyl-3-pyridine carboxylate;
1-methylethyl 4-methyl-2-(4-{[1-(3-pyridinyl)-1H-pyrrol-2-yl]methyl}-1-piperazinyl)-3-pyridinecarboxylate;
1-methylethyl 4-methyl-2-(4-{[4-(1H-tetrazol-5-yl)phenyl]methyl}-1-piperazinyl)-3-pyridinecarboxylate;
1-methylethyl 4-methyl-2-(4-{[4-(methylsulfonyl)phenyl]methyl}-1-piperazinyl)-3-pyridinecarboxylate;
1-methylethyl 2-(4-{[2-[(cyanomethyl)oxy]-3-(methyloxy)phenyl]methyl}-1-piperazinyl)-4-methyl-3-pyridinecarboxylate;
1-methylethyl 4-methyl-2-[4-({1,2,5-trimethyl-4-[(methyloxy)carbonyl]-1H-pyrrol-3-yl]methyl)-1-piperazinyl}-3-pyridinecarboxylate;
1-methylethyl 4-methyl-2-(4-{[2-(1-piperidinyl)-1,3-thiazol-5-yl]methyl}-1-piperazinyl)-3-pyridinecarboxylate;
1-methylethyl 4-methyl-2-(4-{[2-(4-morpholinyl)-1,3-thiazol-5-yl]methyl}-1-piperazinyl)-3-pyridinecarboxylate;
1-methylethyl 4-methyl-2-(4-{[2-(4-methyl-1-piperazinyl)-1,3-thiazol-5-yl]methyl}-1-piperazinyl)-3-pyridinecarboxylate;
1-methylethyl 2-[4-({1-[3-cyano-4-(methyloxy)-2-pyridinyl]-1H-pyrrol-2-yl}methyl)-1-piperazinyl]-4-methyl-3-pyridinecarboxylate;
1-methylethyl 2-(4-{[4-({[3-(trifluoromethyl)phenyl]methyl}oxy)phenyl]methyl}-1-piperazinyl)-3-pyridinecarboxylate;
1-methylethyl 2-{4-[(4-{[(3-bromophenyl)methyl]oxy}phenyl)methyl]-1-piperazinyl}-3-pyridinecarboxylate;
1-methylethyl 2-(4-{[4-{[(2,4-dichlorophenyl)methyl]oxy}-3-(methyloxy)phenyl]methyl}-1-piperazinyl)-3-pyridinecarboxylate;
1-methylethyl 2-[4-({3,5-bis(methyloxy)-4-[(phenylmethyl)oxy]phenyl}methyl)-1-piperazinyl]-3-pyridinecarboxylate;
1-methylethyl 2-[4-({4-(methyloxy)-3-[(phenylmethyl)oxy]phenyl}methyl)-1-piperazinyl]-3-pyridinecarboxylate;
1-methylethyl 2-(4-{[4-{[(4-chlorophenyl)methyl]oxy}-3-(ethyloxy)phenyl]methyl}-1-piperazinyl)-3-pyridinecarboxylate;
1-methylethyl 2-(4-{[4-{[(2-chlorophenyl)methyl]oxy}-3-(methyloxy)phenyl]methyl}-1-piperazinyl)-3-pyridinecarboxylate;
1-methylethyl 2-(4-{[4-{[(2-chlorophenyl)methyl]oxy}-3-(ethyloxy)phenyl]methyl}-1-piperazinyl)-3-pyridinecarboxylate;
1-methylethyl 2-{4-[(4-{[(3-fluorophenyl)methyl]oxy}phenyl)methyl]-1-piperazinyl}-3-pyridinecarboxylate;
1-methylethyl 2-[4-({3-chloro-4-[(phenylmethyl)oxy]phenyl}methyl)-1-piperazinyl]-3-pyridinecarboxylate;
1-methylethyl 2-[4-({2-methyl-4-[(phenylmethyl)oxy]phenyl}methyl)-1-piperazinyl]-3-pyridinecarboxylate;
1-methylethyl 2-{4-[(4-{[(2-chlorophenyl)methyl]oxy}phenyl)methyl]-1-piperazinyl}-3-pyridinecarboxylate;
1-methylethyl 2-[4-({3,5-bis[(phenylmethyl)oxy]phenyl}methyl)-1-piperazinyl]-3-pyridinecarboxylate;
1-methylethyl 2-{4-[(4-{[(4-fluorophenyl)methyl]oxy}phenyl)methyl]-1-piperazinyl}-3-pyridinecarboxylate;
1-methylethyl 2-{4-[(4-{[(2,4-dichlorophenyl)methyl]oxy}phenyl)methyl]-1-piperazinyl}-3-pyridinecarboxylate;
1-methylethyl 2-(4-{[4-{[(4-fluorophenyl)methyl]oxy}-3-(methyloxy)phenyl]methyl}-1-piperazinyl)-3-pyridinecarboxylate;
1-methylethyl 2-[4-({3-(ethyloxy)-4-[(phenylmethyl)oxy]phenyl}methyl)-1-piperazinyl]-3-pyridinecarboxylate;
1-methylethyl 2-[4-({3-(methyloxy)-2-[(phenylmethyl)oxy]phenyl}methyl)-1-piperazinyl]-3-pyridinecarboxylate;
1-methylethyl 2-[4-({4,5-bis(methyloxy)-2-[(phenylmethyl)oxy]phenyl}methyl)-1-piperazinyl]-3-pyridinecarboxylate;
1-methylethyl 2-[4-({4-[(phenylmethyl)oxy]phenyl}methyl)-1-piperazinyl]-3-pyridine carboxylate;
1-methylethyl 2-[4-({3,5-dimethyl-4-[(phenylmethyl)oxy]phenyl}methyl)-1-piperazinyl]-3-pyridinecarboxylate;
1-methylethyl 2-[4-({2-hydroxy-4-[(phenylmethyl)oxy]phenyl}methyl)-1-piperazinyl]-3-pyridinecarboxylate;
1-methylethyl 2-{4-[(4-{[(3,4-dichlorophenyl)methyl]oxy}phenyl)methyl]-1-piperazinyl}-3-pyridinecarboxylate;
1-methylethyl 2-(4-{[4-{[(2-chloro-6-fluorophenyl)methyl]oxy}-3-(methyloxy)phenyl]methyl}-1-piperazinyl)-3-pyridinecarboxylate;
1-methylethyl 2-(4-{[4-{[(4-chlorophenyl)methyl]oxy}-3-(methyloxy)phenyl]methyl}-1-piperazinyl)-3-pyridinecarboxylate;
1-methylethyl 2-(4-{[3-(methyloxy)-4-({[4-(methyloxy)phenyl]methyl}oxy)phenyl]methyl}-1-piperazinyl)-3-pyridinecarboxylate;
1-methylethyl 2-[4-({2-(methyloxy)-4-[(phenylmethyl)oxy]phenyl}methyl)-1-piperazinyl]-3-pyridinecarboxylate;
1-methylethyl 2-{4-[(4-{[(4-bromophenyl)methyl]oxy}phenyl)methyl]-1-piperazinyl}-3-pyridinecarboxylate;
1-methylethyl 2-[4-({2-[(phenylmethyl)oxy]phenyl}methyl)-1-piperazinyl]-3-pyridinecarboxylate;
1-methylethyl 2-[4-({3,4-bis[(phenylmethyl)oxy]phenyl}methyl)-1-piperazinyl]-3-pyridinecarboxylate;
1-methylethyl 2-[4-({3-(methyloxy)-4-[(phenylmethyl)oxy]phenyl}methyl)-1-piperazinyl]-3-pyridinecarboxylate;
1-methylethyl 2-{4-[(4-{[(2-chloro-6-fluorophenyl)methyl]oxy}phenyl)methyl]-1-piperazinyl}-3-pyridinecarboxylate;
1-methylethyl 2-[4-({4-[(4-bromophenyl)oxy]phenyl}methyl)-1-piperazinyl]-3-pyridinecarboxylate;
1-methylethyl 2-[4-({3-[(3,5-dichlorophenyl)oxy]phenyl}methyl)-1-piperazinyl]-3-pyridinecarboxylate;
1-methylethyl 2-[4-({3-[(4-methylphenyl)oxy]phenyl}methyl)-1-piperazinyl]-3-pyridinecarboxylate;
1-methylethyl 2-[4-(2-biphenylylmethyl)-1-piperazinyl]-3-pyridinecarboxylate;
1-methylethyl 2-[4-({4-[(3-chlorophenyl)oxy]phenyl}methyl)-1-piperazinyl]-3-pyridinecarboxylate;
1-methylethyl 2-(4-{[4-fluoro-3-(phenyloxy)phenyl]methyl}-1-piperazinyl)-3-pyridinecarboxylate;
1-methylethyl 2-[4-({3-[(4-chlorophenyl)oxy]phenyl}methyl)-1-piperazinyl]-3-pyridinecarboxylate;
1-methylethyl 2-[4-(9H-fluoren-2-ylmethyl)-1-piperazinyl]-3-pyridinecarboxylate;
1-methylethyl 2-[4-(4-biphenylylmethyl)-1-piperazinyl]-3-pyridinecarboxylate;
1-methylethyl 2-[4-({4-[(4-methylphenyl)oxy]phenyl}methyl)-1-piperazinyl]-3-pyridinecarboxylate;
1-methylethyl 2-(4-{[3-(phenyloxy)phenyl]methyl}-1-piperazinyl)-3-pyridinecarboxylate;
1-methylethyl 2-[4-({3-[(3,4-dichlorophenyl)oxy]phenyl}methyl)-1-piperazinyl]-3-pyridinecarboxylate;
1-methylethyl 2-{4-[(4′-methyl-3-biphenyl)methyl]-1-piperazinyl}-3-pyridinecarboxylate;
1-methylethyl 2-[4-({2-[(4-cyanophenyl)oxy]phenyl}methyl)-1-piperazinyl]-3-pyridinecarboxylate;
1-methylethyl 2-{4-[(4′-methyl-4-biphenylyl)methyl]-1-piperazinyl}-3-pyridine carboxylate;
1-methylethyl 2-[4-({4-[(4-fluorophenyl)oxy]phenyl}methyl)-1-piperazinyl]-3-pyridinecarboxylate;
1-methylethyl 2-{4-[(9-ethyl-9H-carbazol-3-yl)methyl]-1-piperazinyl}-3-pyridine carboxylate;
1-methylethyl 2-[4-(dibenzo[b,d]furan-4-ylmethyl)-1-piperazinyl]-3-pyridine carboxylate;
1-methylethyl 2-[4-({4-[(4-chlorophenyl)oxy]phenyl}methyl)-1-piperazinyl]-3-pyridinecarboxylate;
1-methylethyl 2-{4-[(4′-chloro-3-biphenylyl)methyl]-1-piperazinyl}-3-pyridine carboxylate;
1-methylethyl 2-{4-[(2-{[4-(methyloxy)phenyl]oxy}phenyl)methyl]-1-piperazinyl}-3-pyridinecarboxylate;
1-methylethyl 2-[4-({4-[(2,4-dichlorophenyl)oxy]phenyl}methyl)-1-piperazinyl]-3-pyridinecarboxylate;
1-methylethyl 2-{4-[(4-{[4-(methyloxy)phenyl]oxy}phenyl)methyl]-1-piperazinyl}-3-pyridinecarboxylate;
1-methylethyl 2-[4-({2-[(4-fluorophenyl)oxy]phenyl}methyl)-1-piperazinyl]-3-pyridinecarboxylate;
1-methylethyl 2-[4-({2-[(4-chlorophenyl)oxy]phenyl}methyl)-1-piperazinyl]-3-pyridinecarboxylate;
1-methylethyl 2-[4-({4′-[(methyloxy)carbonyl]-3-biphenylyl]methyl)-1-piperazinyl}-3-pyridinecarboxylate;
1-methylethyl 2-[4-({4′-[(methyloxy)carbonyl]-4-biphenylyl]methyl)-1-piperazinyl}-3-pyridinecarboxylate;
1-methylethyl 2-[4-({4-[(4-cyanophenyl)oxy]phenyl}methyl)-1-piperazinyl]-3-pyridinecarboxylate;
1-methylethyl 2-{4-[(3-{[4-(methyloxy)phenyl]oxy}phenyl)methyl]-1-piperazinyl}-3-pyridinecarboxylate;
1-methylethyl 2-(4-{[4-(phenyloxy)phenyl]methyl}-1-piperazinyl)-3-pyridine carboxylate;
1-methylethyl 2-{4-[(3-{[4-(1,1-dimethylethyl)phenyl]oxy}phenyl)methyl]-1-piperazinyl}-3-pyridinecarboxylate;
1-methylethyl 2-(4-{[2′-(trifluoromethyl)-3-biphenylyl]methyl}-1-piperazinyl)-3-pyridinecarboxylate;
1-methylethyl 2-[4-({2-[(4-chlorophenyl)thio]phenyl}methyl)-1-piperazinyl]-3-pyridinecarboxylate;
1-methylethyl 2-(4-{[2′-(trifluoromethyl)-4-biphenylyl]methyl}-1-piperazinyl)-3-pyridinecarboxylate;
1-methylethyl 2-(4-{[3′-(methyloxy)-2-biphenylyl]methyl}-1-piperazinyl)-3-pyridine carboxylate;
1-methylethyl 2-{4-[(3-{[3-(trifluoromethyl)phenyl]oxy}phenyl)methyl]-1-piperazinyl}-3-pyridinecarboxylate;
1-methylethyl 2-(4-{[2-(phenyloxy)phenyl]methyl}-1-piperazinyl)-3-pyridine carboxylate;
1-methylethyl 2-(4-{[3-({[3,4-bis(methyloxy)phenyl]methyl}oxy)phenyl]methyl}-1-piperazinyl)-3-pyridinecarboxylate;
1-methylethyl 2-(4-{[3-({[3,4-(1,1-dimethylethyl)phenyl]methyl}oxy)phenyl]methyl}-1-piperazinyl)-3-pyridinecarboxylate;
1-methylethyl 2-(4-{[3-({[3,5-bis(methyloxy)phenyl]methyl}oxy)phenyl]methyl}-1-piperazinyl)-3-pyridinecarboxylate;
1-methylethyl 2-{4-[(3-{[(2,4,5-trifluorophenyl)methyl]oxy}phenyl)methyl]-1-piperazinyl}-3-pyridinecarboxylate;
1-methylethyl 2-[4-({3-[(2,3-dihydro-1,4-benzodioxin-5-ylmethyl)oxy]phenyl}methyl)-1-piperazinyl]-3-pyridinecarboxylate;
1-methylethyl 2-(4-{[3-({[2-(methyloxy)phenyl]methyl}oxy)phenyl]methyl}-1-piperazinyl)-3-pyridinecarboxylate;
1-methylethyl 2-{4-[(3-{[(2,6-difluorophenyl)methyl]oxy}phenyl)methyl]-1-piperazinyl}-3-pyridinecarboxylate;
1-methylethyl 2-{4-[(3-{[(3,5-dimethylphenyl)methyl]oxy}phenyl)methyl]-1-piperazinyl}-3-pyridinecarboxylate;
1-methylethyl 2-(4-{[3-({[3-(dimethylamino)phenyl]methyl}oxy)phenyl]methyl}-1-piperazinyl)-3-pyridinecarboxylate;
1-methylethyl 2-{4-[(3-{[(2,4-difluorophenyl)methyl]oxy}phenyl)methyl]-1-piperazinyl}-3-pyridinecarboxylate;
1-methylethyl 2-{4-[(3-{[(2,3-difluorophenyl)methyl]oxy}phenyl)methyl]-1-piperazinyl}-3-pyridinecarboxylate;
1-methylethyl 2-(4-{[3-({[4-(butyloxy)phenyl]methyl}oxy)phenyl]methyl}-1-piperazinyl)-3-pyridinecarboxylate;
1-methylethyl 2-(4-{[3-({[4-(ethyloxy)phenyl]methyl}oxy)phenyl]methyl}-1-piperazinyl)-3-pyridinecarboxylate;
1-methylethyl 2-{4-[(3-{[(4-ethylphenyl)methyl]oxy}phenyl)methyl]-1-piperazinyl}-3-pyridinecarboxylate;
1-methylethyl 2-(4-{[3-({[2-fluoro-6-(methyloxy)phenyl]methyl}oxy)phenyl]methyl}-1-piperazinyl)-3-pyridinecarboxylate;
1-methylethyl 2-{4-[(3-{[(4-cyanophenyl)methyl]oxy}phenyl)methyl]-1-piperazinyl}-3-pyridinecarboxylate;
1-methylethyl 2-{4-[(3-{[(2,4-dimethylphenyl)methyl]oxy}phenyl)methyl]-1-piperazinyl}-3-pyridinecarboxylate;
1-methylethyl 2-(4-{[3-({[4-fluoro-3-(methyloxy)phenyl]methyl}oxy)phenyl]methyl}-1-piperazinyl)-3-pyridinecarboxylate;
1-methylethyl 2-[4-({3-[(1-naphthalenylmethyl)oxy]phenyl}methyl)-1-piperazinyl]-3-pyridinecarboxylate;
1-methylethyl 2-(4-{[3-({[4-(methylsulfonyl)phenyl]methyl}oxy)phenyl]methyl}-1-piperazinyl)-3-pyridinecarboxylate;
1-methylethyl 2-{4-[(3-{[(3,5-dichlorophenyl)methyl]oxy}phenyl)methyl]-1-piperazinyl}-3-pyridinecarboxylate;
1-methylethyl 2-{4-[(3-{[(2,3-dichlorophenyl)methyl]oxy}phenyl)methyl]-1-piperazinyl}-3-pyridinecarboxylate;
1-methylethyl 2-[4-({3-[({4-[(methyloxy)carbonyl]phenyl}methyl)oxy]phenyl}methyl)-1-piperazinyl]-3-pyridinecarboxylate;
1-methylethyl 2-(4-{[3-({[4-chloro-2-(methyloxy)phenyl]methyl}oxy)phenyl]methyl}-1-piperazinyl)-3-pyridinecarboxylate;
1-methylethyl 2-(4-{[3-({[4-(methyloxy)phenyl]methyl}oxy)phenyl]methyl}-1-piperazinyl)-3-pyridinecarboxylate;
1-methylethyl 2-(4-{[3-({[4-(1-methylethyl)phenyl]methyl}oxy)phenyl]methyl}-1-piperazinyl)-3-pyridinecarboxylate;
1-methylethyl 2-(4-{[3-({[2,5-bis(methyloxy)phenyl]methyl}oxy)phenyl]methyl}-1-piperazinyl)-3-pyridinecarboxylate;
1-methylethyl 2-(4-{[3-({[2,4-bis(methyloxy)phenyl]methyl}oxy)phenyl]methyl}-1-piperazinyl)-3-pyridinecarboxylate;
1-methylethyl 2-(4-{[4-({[3,4-bis(methyloxy)phenyl]methyl}oxy)phenyl]methyl}-1-piperazinyl)-3-pyridinecarboxylate;
1-methylethyl 2-(4-{[4-({[4-(1,1-dimethylethyl)phenyl]methyl}oxy)phenyl]methyl}-1-piperazinyl)-3-pyridinecarboxylate;
1-methylethyl 2-{4-[(4-{[(3-chlorophenyl)methyl]oxy}phenyl)methyl]-1-piperazinyl}-3-pyridinecarboxylate;
1-methylethyl 2-(4-{[4-({[3,5-bis(methyloxy)phenyl]methyl}oxy)phenyl]methyl}-1-piperazinyl)-3-pyridinecarboxylate;
1-methylethyl 2-{4-[(4-{[(2,4,5-trifluorophenyl)methyl]oxy}phenyl)methyl]-1-piperazinyl}-3-pyridinecarboxylate;
1-methylethyl 2-[4-({4-[(2,3-dihydro-1,4-benzodioxin-5-ylmethyl)oxy]phenyl}methyl)-1-piperazinyl]-3-pyridinecarboxylate;
1-methylethyl 2-(4-{[4-({[2-(methyloxy)phenyl]methyl}oxy)phenyl]methyl}-1-piperazinyl)-3-pyridinecarboxylate;
1-methylethyl 2-{4-[(4-{[(2,6-difluorophenyl)methyl]oxy}phenyl)methyl]-1-piperazinyl}-3-pyridinecarboxylate;
1-methylethyl 2-{4-[(4-{[(3,5-dimethylphenyl)methyl]oxy}phenyl)methyl]-1-piperazinyl}-3-pyridinecarboxylate;
1-methylethyl 2-{4-[(4-{[(2-ethylphenyl)methyl]oxy}phenyl)methyl]-1-piperazinyl}-3-pyridinecarboxylate;
1-methylethyl 2-(4-{[4-({[3-(dimethylamino)phenyl]methyl}oxy}phenyl)methyl]-1-piperazinyl)-3-pyridinecarboxylate;
1-methylethyl 2-{4-[(4-{[(2,4-difluorophenyl)methyl]oxy}phenyl)methyl]-1-piperazinyl}-3-pyridinecarboxylate;
1-methylethyl 2-{4-[(4-{[(3-methylphenyl)methyl]oxy}phenyl)methyl]-1-piperazinyl}-3-pyridinecarboxylate;
1-methylethyl 2-{4-[(4-{[(3,4-difluorophenyl)methyl]oxy}phenyl)methyl]-1-piperazinyl}-3-pyridinecarboxylate;
1-methylethyl 2-(4-{[4-({[4-(butyloxy)phenyl]methyl}oxy)phenyl]methyl}-1-piperazinyl)-3-pyridinecarboxylate;
1-methylethyl 2-(4-{[4-({[4-(ethyloxy)phenyl]methyl}oxy)phenyl]methyl}-1-piperazinyl)-3-pyridinecarboxylate;
1-methylethyl 2-{4-[(4-{[(4-ethylphenyl)methyl]oxy}phenyl)methyl]-1-piperazinyl}-3-pyridinecarboxylate;
1-methylethyl 2-(4-{[4-({[2-fluoro-6-(methyloxy)phenyl]methyl}oxy)phenyl]methyl}-1-piperazinyl)-3-pyridinecarboxylate;
1-methylethyl 2-{4-[(4-{[(5-chloro-2-fluorophenyl)methyl]oxy}phenyl)methyl]-1-piperazinyl}-3-pyridinecarboxylate;
1-methylethyl 2-{4-[(4-{[(4-cyanophenyl)methyl]oxy}phenyl)methyl]-1-piperazinyl}-3-pyridinecarboxylate;
1-methylethyl 2-{4-[(4-{[(4-methylphenyl)methyl]oxy}phenyl)methyl]-1-piperazinyl}-3-pyridinecarboxylate;
1-methylethyl 2-{4-[(4-{[(2,6-dichlorophenyl)methyl]oxy}phenyl)methyl]-1-piperazinyl}-3-pyridinecarboxylate;
1-methylethyl 2-(4-{[4-({[2-(ethyloxy)phenyl]methyl}oxy)phenyl]methyl}-1-piperazinyl)-3-pyridinecarboxylate;
1-methylethyl 2-{4-[(4-{[(2,4-dimethylphenyl)methyl]oxy}phenyl)methyl]-1-piperazinyl}-3-pyridinecarboxylate;
1-methylethyl 2-(4-{[4-({[4-fluoro-3-(methyloxy)phenyl]methyl}oxy)phenyl]methyl}-1-piperazinyl)-3-pyridinecarboxylate;
1-methylethyl 2-[4-({4-[(1-naphthalenylmethyl)oxy]phenyl}methyl)-1-piperazinyl]-3-pyridinecarboxylate;
1-methylethyl 2-(4-{[4-({[4-(methylsulfonyl)phenyl]methyl}oxy)phenyl]methyl}-1-piperazinyl)-3-pyridinecarboxylate;
1-methylethyl 2-[4-({4-[(2-biphenylylmethyl)oxy]phenyl}methyl)-1-piperazinyl]-3-pyridinecarboxylate;
1-methylethyl 2-{4-[(4-{[(3,5-dichlorophenyl)methyl]oxy}phenyl)methyl]-1-piperazinyl}-3-pyridinecarboxylate;
1-methylethyl 2-{4-[(4-{[(2,3-dichlorophenyl)methyl]oxy}phenyl)methyl]-1-piperazinyl}-3-pyridinecarboxylate;
1-methylethyl 2-[4-({4-[({4-[(methyloxy)carbonyl]phenyl}methyl)oxy]phenyl}methyl)-1-piperazinyl]-3-pyridinecarboxylate;
1-methylethyl 2-(4-{[4-({[4-chloro-2-(methyloxy)phenyl]methyl}oxy)phenyl]methyl}-1-piperazinyl)-3-pyridinecarboxylate;
1-methylethyl 2-(4-{[4-({[4-(methyloxy)phenyl]methyl}oxy)phenyl]methyl}-1-piperazinyl)-3-pyridinecarboxylate;
1-methyl ethyl 2-{4-[(4-{[(2-methylphenyl)methyl]oxy}phenyl)methyl]-1-piperazinyl}-3-pyridinecarboxylate;
1-methylethyl 2-(4-{[4-({[4-(1-methylethyl)phenyl]methyl}oxy)phenyl]methyl}-1-piperazinyl)-3-pyridinecarboxylate;
1-methylethyl 2-[4-({4-[(4-biphenylylmethyl)oxy]phenyl}methyl)-1-piperazinyl]-3-pyridinecarboxylate;
1-methylethyl 2-(4-{[4-({[2,5-bis(methyloxy)phenyl]methyl}oxy)phenyl]methyl}-1-piperazinyl)-3-pyridinecarboxylate;
1-methylethyl 2-(4-{[4-({[3-(methyloxy)phenyl]methyl}oxy)phenyl]methyl}-1-piperazinyl)-3-pyridinecarboxylate;
1-methylethyl 2-(4-{[4-({[2,4-bis(methyloxy)phenyl]methyl}oxy)phenyl]methyl}-1-piperazinyl)-3-pyridinecarboxylate;
1-methylethyl 2-(4-{[4-({[2-(trifluoromethyl)phenyl]methyl}oxy)phenyl]methyl}-1-piperazinyl)-3-pyridinecarboxylate;
1-methylethyl 2-(4-{[4-({4-[(2-chloro-6-fluorophenyl)methyl]-1-piperazinyl}methyl)phenyl]methyl}1-piperazinyl)-3-pyridinecarboxylate;
1-methylethyl 2-{4-[(4-{[4-(phenylmethyl)-1-piperazinyl]methyl}phenyl)methyl]-1-piperazinyl}-3-pyridinecarboxylate; 1-methylethyl 2-{4-[(4-{[4-(2-pyridinylmethyl)-1-piperazinyl]methyl}phenyl)methyl]-1-piperazinyl}-3-pyridinecarboxylate; 1-methylethyl 2-[4-({4-[(4-{[3-(methyloxy)phenyl]methyl}-1-piperazinyl)methyl]phenyl}methyl)-1-piperazinyl]-3-pyridinecarboxylate; 1-methylethyl 2-[4-({4-[(4-{[4-(methyloxy)phenyl]methyl}-1-piperazinyl)methyl]phenyl}methyl)-1-piperazinyl]-3-pyridinecarboxylate;
1-Methylethyl-2-{4-[(3-{[4-(methyloxy)phenyl]oxy}phenyl)methyl]-1-piperazinyl}-3-pyridine carboxylate dihydrochloride;
1-Methylethyl-2-(4-{[2′-(trifluoromethyl)-3-biphenylyl]methyl}-1-piperazinyl)-3-pyridine carboxylate; 1-Methylethyl-2-(4-{[3-({[2-(methyloxy)phenyl]methyl}oxy)phenyl]methyl}-1-piperazinyl)-3-pyridinecarboxylate hydrochloride;
1-Methylethyl-2-(4-{[3-({[4-(ethyloxy)phenyl]methyl}oxy)phenyl]methyl}-1-piperazinyl)-3-pyridinecarboxylate;
1-Methylethyl 2-[4-({4-[(ethylamino)methyl]phenyl}methyl)-1-piperazinyl]-3-pyridine carboxylate dihydrochloride;
1-Methylethyl 2-{4-[(4-{[[(2-chloro-6-fluorophenyl)methyl](ethyl)amino]methyl}phenyl)methyl]-1-piperazinyl}-3-pyridinecarboxylate;
1-[(4-{[[(2-Chloro-6-fluorophenyl)methyl](ethyl)ammonio]methyl}phenyl)methyl]-4-(3-{[(1-methylethyl)oxy]carbonyl}-2-pyridinyl)piperazin-1-ium di-maleate;
1-methylethyl 2-(4-{[4-({ethyl[(2-{[(1-methylethyl)oxy]carbonyl}phenyl)methyl]amino}methyl)phenyl]methyl}-1-piperazinyl)-3-pyridinecarboxylate dihydrochloride;
1-methylethyl 2-(4-{[4-({ethyl[(3-{[(1-methylethyl)oxy]carbonyl}phenyl)methyl]amino}methyl)phenyl]methyl}-1-piperazinyl)-3-pyridinecarboxylate dihydrochloride;
1-methylethyl 2-(4-{[4-({ethyl[(4-{[(1-methylethyl)oxy]carbonyl}phenyl)methyl]amino}methyl)phenyl]methyl}-1-piperazinyl)-3-pyridinecarboxylate;
1-methylethyl 2-[4-({2-[(dimethylamino)sulfonyl]phenyl}methyl)-1-piperazinyl]-3-pyridine carboxylate hydrochloride;
1-Methylethyl 2-[4-({3-[(dimethylamino)sulfonyl]phenyl}methyl)-1-piperazinyl]-3-pyridine carboxylate;
1-methylethyl 2-[4-({4-[(dimethylamino)sulfonyl]phenyl}methyl)-1-piperazinyl]-3-pyridine carboxylate;
1-methylethyl 2-{4-[(4-{[({2-[(dimethylamino)sulfonyl]phenyl}methyl}(ethyl)amino]methyl}phenyl)methyl]-1-piperazinyl)-3-pyridinecarboxylate;
1-methylethyl 2-{4-[(4-{[({3-[(dimethylamino)sulfonyl]phenyl}methyl)(ethyl)amino]methyl}phenyl)methyl]-1-piperazinyl}-3-pyridinecarboxylate; 1-methylethyl2-{4-[(4-{[({4[(dimethylamino)sulfonyl]phenyl}methyl)(ethyl)amino]methyl}phenyl)methyl]-1-piperazinyl}-3-pyridinecarboxylate; 1-Methylethyl 2-{4-{[[2-(2-chloro-6-fluorophenyl)ethyl](ethyl)amino]methyl}phenyl)methyl}-1-piperazinyl)-3-pyridinecarboxylate dihydrochloride;
1-methylethyl 2-{4-[(4-{[[(2-chloro-6-fluorophenyl)methyl](ethyl)amino]methyl}phenyl)methyl]-1-piperazinyl}-3-pyridinecarboxylate dihydrochloride hydrochloride;
1-methylethyl 2-(4-{[4-({ethyl[(3-fluorophenyl)methyl]amino}methyl)phenyl]methyl}-1-piperazinyl)-3-pyridinecarboxylate;
1-methylethyl 2-(4-{[4-({ethyl[(4-fluorophenyl)methyl]amino}methyl)phenyl]methyl}-1-piperazinyl)-3-pyridinecarboxylate;
1-methylethyl 2-{4-[(4-{[[(2,6-difluorophenyl)methyl](ethyl)amino]methyl}phenyl)methyl]-1-piperazinyl}-3-pyridinecarboxylate; 1-methylethyl 2-(4-{[4-({ethyl[(2-fluorophenyl)methyl]amino}methyl)phenyl]methyl}-1-piperazinyl)-3-pyridinecarboxylate; 1-methylethyl 2-{4-[(4-{[[(2,6-dichlorophenyl)methyl](ethyl)amino]methyl}phenyl)methyl]-1-piperazinyl}-3-pyridinecarboxylate; 1-methylethyl 2-{4-[(4-{[[(3-chlorophenyl)methyl](ethyl)amino]methyl}phenyl)methyl]-1-piperazinyl}-3-pyridinecarboxylate; 1-methylethyl 2-{4-[(4-{[ethyl(phenylmethyl)amino]methyl}phenyl)methyl]-1-piperazinyl}-3-pyridine carboxylate;
1-methylethyl 2-{4-[(4-{[[(4-chlorophenyl)methyl](ethyl)amino]methyl}phenyl)methyl]-1-piperazinyl}-3-pyridinecarboxylate;
1-methylethyl2-{4-[(4-{[[(2-chlorophenyl)methyl](ethyl)amino]methyl}phenyl)methyl]-1-piperazinyl}-3-pyridinecarboxylate;
1-methylethyl2-(4-{[4-({ethyl[(6-methyl-2-pyridinyl)methyl]amino}methyl)phenyl]methyl}-1-piperazinyl)-3-pyridinecarboxylate;
1-methylethyl2-(4-{[4-({[(2-chloro-6-fluorophenyl)methyl]amino}methyl)phenyl]methyl}-1-piperazinyl)-3-pyridinecarboxylate;
1-methylethyl2-{4-[(4-{[[(2-chloro-6-fluorophenyl)carbonyl](ethyl)amino]methyl}phenyl)methyl]-1-piperazinyl}-3-pyridinecarboxylate;
1-methylethyl2-(4-{[4-({[(2-chloro-6-fluorophenyl)methyl][3-(2-oxo-1-pyrrolidinyppropyl]amino}methyl)phenyl]methyl}-1-piperazinyl)-3-pyridinecarboxylate;
1-methylethyl2-{4-[(3-{[[(2-chloro-6-fluorophenyl)methyl](ethyl)amino]methyl}phenyl)methyl]-1-piperazinyl}-3-pyridinecarboxylate;
1-methylethyl2-(4-{[4-({ethyl[(2-methyl-3-pyridinyl)methyl]amino}methyl)phenyl]methyl}-1-piperazinyl)-3-pyridinecarboxylate;
1-methylethyl2-(4-{[4-({[(2-fluorophenyl)methyl]amino}methyl)phenyl]; methyl}-1-piperazinyl)-3-pyridinecarboxylate;
1-methylethyl2-{4-[(2-{[[(2-chloro-6-fluorophenyl)methyl](ethyl)amino]; methyl}phenyl)methyl]-1-piperazinyl}-3-pyridinecarboxylate;
1-methylethyl2-{4-[(4-{[[3-(2-chloro-6-fluorophenyl)propyl](ethyl)amino]methyl}phenyl)methyl]-1-piperazinyl}-3-pyridinecarboxylate;
1-methylethyl 2-{4-[(4-{[(phenylmethyl)amino]methyl}phenyl)methyl]-1-piperazinyl}-3-pyridinecarboxylate;
1-methylethyl 2-(4-{[4-({ethyl[(2-fluorophenyl)methyl]amino}methyl)phenyl]methyl}-1-piperazinyl)-3-pyridinecarboxylate dihydrochloride;
1-methylethyl 2-{4-[(4-{[ethyl(phenylmethyl)amino]methyl}phenyl)methyl]-1-piperazinyl}-3-pyridine carboxylate dihydrochloride;
1-methylethyl 2-(4-{[4-({[(2-chloro-6-fluorophenyl)carbonyl]amino}methyl)phenyl]methyl}-1-piperazinyl)-3-pyridinecarboxylate;
1-methylethyl 2-(4-{[4-({ethyl[(6-methyl-2-pyridinyl)methyl]amino}methyl)phenyl]methyl}-1-piperazinyl)-3-pyridine carboxylate quaternary hydrochloride1-methylethyl 2-(4-{[4-({[(2-fluorophenyl)carbonyl]amino}methyl)phenyl}methyl]-1-piperazinyl)-3-pyridinecarboxylate;
1-methylethyl 2-{4-[(4-{[(phenylcarbonyl)amino]methyl}phenyl)methyl]-1-piperazinyl}-3-pyridinecarboxylate;
1-methylethyl 2-(4-{[4-({[(2-chloro-6-fluorophenyl)methyl]oxy}methyl)phenyl]methyl}-1-piperazinyl)-3-pyridinecarboxylate;
1-methylethyl 2-(4-{[4-({[(2-chloro-6-fluorophenyl)methyl]amino}methyl)phenyl]methyl}-1-piperazinyl)-3-pyridine carboxylate trihydrochloride; 1-methylethyl2-{4-[(4-{[[(2-chloro-6-fluorophenyl)carbonyl](ethyl)amino]methyl}phenyl)methyl]-1-piperazinyl}-3-pyridinecarboxylate dihydrochloride; or
   a pharmaceutically acceptable salt thereof.

In another aspect, the present invention relates to a compound of Formula (V):

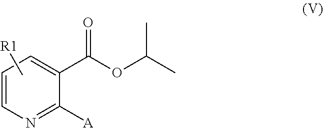

wherein:
R₁ is H, halogen, straight or branched C₁₋₆ alkyl, phenyl, substituted phenyl, —NHR₁ₐ, —SR_{1b} or —OR_{1c};

wherein:

R₁ₐ, R_{1b} or R_{1c} as defined in R₁ above is phenyl or substituted phenyl;

### A is:

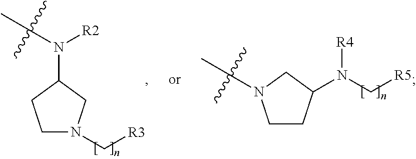

wherein:

n is 0 or an integer from 1 to 5;
R₂ is H, straight or branched C₁₋₆ alkyl or (CH₂)ₓ-cycloalkyl;
R₃ is phenyl or thienyl;

wherein R₃ optionally is substituted with at least one of the following substitutents straight or branched C₁₋₆ alkyl, straight or branched C₁₋₆ haloalkyl, C₁₋₆-alkoxy, straight or branched C₁₋₆-halosubstituted alkoxy, phenyl, phenoxy, benzyloxy, 3-pyridinyl or 2-thienyl;
wherein phenoxy or benzyloxy optionally is substituted by at least one of following substituents: halogen, —CN, straight or branched C₁₋₆ alkyl, straight or branched C₁₋₆-alkoxy, —O(CH₂)ₙC(O)—N(Ra)₂, SO₂R_{b}; —C(O)R_{c};
   wherein:
   Rₐ is H, alkyl or cycloalkyl;
   R_{b} is NH₂, alkyl, cycloalkyl, aryl, heteroaryl;
   R_{c} is straight or branched C₁₋₆ alkyl;
      R₄ is H, straight or branched C₁₋₆ alkyl; cycloalkyl, (CH₂)ₓ-cycloalkyl, (CH₂)ₓ-heterocycloalkyl;
      R₅ is phenyl, furanyl, thienyl, piperidinyl, or pyridinyl;

wherein R₅ is optionally substituted with at least one of the following substitutents: phenyl, phenoxy, pyridinyl or thienyl;
wherein phenyl, phenoxy, pyridinyl or thienyl as defined for R₅ further is optionally substituted by at least one of the following substituents: halogen, straight or branched C₁₋₆ alkyl, straight, straight or branched C₁₋₆ haloalkyl, branched C₁₋₆-alkoxy, —O(CH₂)ₙC(O)Rₓ, phenyl, substituted phenyl, phenoxy, benzyloxy, pyridinyl, thienyl or piperidinyl;
wherein:
Rₓ is straight or branched C₁₋₆ alkyl
benzyloxy, phenoxy, substituted phenyl is optionally substituted by at least one of the following substituents halogen, —CN, straight or branched C₁₋₆ alkyl straight or branched C₁₋₆-alkoxy, or
   a pharmaceutically acceptable salt thereof.

In another aspect, the present invention relates to a compound of Formula (VI):

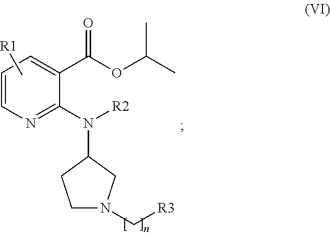

wherein:
n is 0 or an integer from 1 to 5;

### R₁ is H;

R₂ is C₁₋₆ alkyl, cycloalkyl or (CH₂)ₓ-cycloalkyl;
R₃ is C₁₋₆ alkyl, alkoxyalkyl, phenyl, heteroaryl;

wherein R₃ optionally is substituted with at least one of following substitutents:

straight or branched C₁₋₆ alkyl, straight or branched C₁₋₆ haloalkyl, C₁₋₆-alkoxy, phenyl, phenoxy or benzyloxy, heteroaryl, heteroaryloxy;
wherein phenoxy or benzyloxy optionally is substituted by at least one of following substituents: halogen, —CN, straight or branched C₁₋₆ alkyl, straight or branched C₁₋₆-alkoxy; or
   a pharmaceutically acceptable salt thereof.

In another aspect, the present invention relates to a compound of Formula (VII):

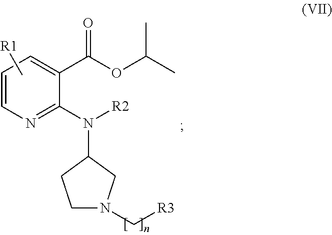

wherein:
n is 0 or an integer from 1 to 5;

### R₁ is H;

R₂ is C₁₋₆ alkyl, cycloalkyl or (CH₂)ₓ-cycloalkyl;
R₃ is C₁₋₆ alkyl, alkoxyalkyl, phenyl, heteroaryl;

wherein:
x is 0 or an integer from 1 to 5;
R₃ optionally is substituted with at least one of following substitutents:

straight or branched C₁₋₆ alkyl, straight or branched C₁₋₆ haloalkyl, C₁₋₆-alkoxy, phenyl, phenoxy or benzyloxy;
wherein:
phenoxy or benzyloxy optionally is substituted by at least one of following substituents: halogen, —CN, straight or branched C₁₋₆ alkyl, straight or branched C₁₋₆-alkoxy; or
   a pharmaceutically acceptable salt thereof.

In another aspect, in a compound of formula (VII), R₂ is methyl or ethyl; R₃ is phenyl or 2-thienyl; halogen is selected from fluoro or chloro.

In another aspect, the present invention relates to a compound which is:
1-methylethyl 2-[methyl((3S)-1-{[3-(phenyloxy)phenyl]methyl}-3-pyrrolidinyl)amino]-3-pyridinecarboxylate;
1-methylethyl 2-{methyl[(3S)-1-({4-[(phenylmethyl)oxy]phenyl}methyl)-3-pyrrolidinyl]amino}-3-pyridinecarboxylate;
1-methylethyl 2-{methyl[(3S)-1-({3-[(phenylmethyl)oxy]phenyl}methyl)-3-pyrrolidinyl]amino}-3-pyridinecarboxylate;
1-methylethyl 2-{methyl[(3S)-1-({3-(methyloxy)-4-[(phenylmethyl)oxy]phenyl}methyl)-3-pyrrolidinyl]amino}-3-pyridinecarboxylate;
1-methylethyl 2-[((3S)-1-{[4-(hexyloxy)phenyl]methyl}-3-pyrrolidinyl)(methyl)amino]-3-pyridinecarboxylate;
1-methylethyl 2-[methyl((3S)-1-{[4-(propyloxy)phenyl]methyl}-3-pyrrolidinyl)amino]-3-pyridinecarboxylate;
1-methylethyl 2-{methyl[(3S)-1-({2-[(trifluoromethyl)oxy]phenyl}methyl)-3-pyrrolidinyl]amino}-3-pyridinecarboxylate;
1-methylethyl 2-(methyl{(3S)-1-[(2-methylphenyl)methyl]-3-pyrrolidinyl}amino)-3-pyridinecarboxylate;
1-methylethyl 2-[[(3S)-1-(2-biphenylylmethyl)-3-pyrrolidinyl](methyl)amino]-3-pyridinecarboxylate;
1-methylethyl 2-[((3S)-1-{[4-{[(2-chloro-6-fluorophenyl)methyl]oxy}-3-(methyloxy)phenyl]methyl}-3-pyrrolidinyl)(methyl)amino]-3-pyridinecarboxylate;
1-methylethyl 2-[{(3S)-1-[(5-ethyl-2-thienyl)methyl]-3-pyrrolidinyl}(methyl)amino]-3-pyridinecarboxylate;
1-methylethyl 2-(methyl{(3S)-1-[(3-{[(4-methylphenyl)methyl]oxy}phenyl)methyl]-3-pyrrolidinyl}amino)-3-pyridinecarboxylate;
1-methylethyl 2-[{(3S)-1-[(3-{[(3-fluorophenyl)methyl]oxy}phenyl)methyl]-3-pyrrolidinyl}(methyl)amino]-3-pyridinecarboxylate;
1-methylethyl 2-{methyl[(3S)-1-({3-(methyloxy)-2-[(phenylmethyl)oxy]phenyl}methyl)-3-pyrrolidinyl]amino}-3-pyridinecarboxylate;
1-methylethyl 2-[{(3S)-1-[(3-{[(2-chlorophenyl)methyl]oxy}phenyl)methyl]-3-pyrrolidinyl}(methyl)amino]-3-pyridinecarboxylate;
1-methylethyl 2-[[(3S)-1-({2-[(4-chlorophenyl)oxy]phenyl}methyl)-3-pyrrolidinyl](methyl)amino]-3-pyridinecarboxylate;
1-methylethyl 2-{methyl[(3S)-1-({4-[(4-methylphenyl)oxy]phenyl}methyl)-3-pyrrolidinyl}amino]-3-pyridinecarboxylate;
1-methylethyl 2-(methyl{(3S)-1-[(2-{[4-(methyloxy)phenyl]oxy}phenyl)methyl]-3-pyrrolidinyl}amino)-3-pyridinecarboxylate;
1-methylethyl 2-[[(3S)-1-({4-[(4-cyanophenyl)oxy]phenyl}methyl)-3-pyrrolidinyl](methyl)amino]-3-pyridinecarboxylate;
1-methylethyl 2-[{(3S)-1-[(4-{[(2-chloro-6-fluorophenyl)methyl]oxy}phenyl)methyl]-3-pyrrolidinyl}(methyl)amino]-3-pyridinecarboxylate;
1-methylethyl 2-[{(3S)-1-[(3-{[(2-chloro-6-fluorophenyl)methyl]oxy}phenyl) methyl]-3-pyrrolidinyl}(methyl)amino]-3-pyridinecarboxylate; or
   a pharmaceutically acceptable salt thereof.

In another aspect, the present invention, relates to a compound of Formula (VIIIA):

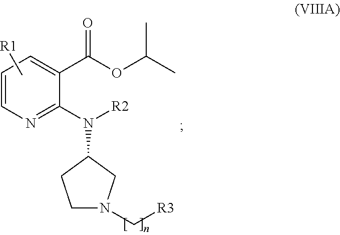

wherein:
n is 1

### R₁ is H;

R₂ is C₁₋₆ alkyl, cycloalkyl or (CH₂)ₓ-cycloalkyl;

wherein:

x is 0 or an integer from 1 to 5;

R₃ is C₁₋₆ alkyl, alkoxyalkyl, phenyl, heteroaryl;

wherein:
R₃ optionally is substituted with at least one of following substitutents: straight or branched C₁₋₆ alkyl, straight or branched C₁₋₆ haloalkyl, straight or branched C₁₋₆-alkoxy, straight or branched C₁₋₆-halosubstituted alkoxy, phenyl. phenoxy, benzyloxy, 3-pyridinyl or 2-thienyl;
wherein:
phenoxy or benzyloxy optionally is substituted by at least one of following substituents: halogen, —CN, straight or branched C₁₋₆ alkyl, straight or branched C₁₋₆-alkoxy, —O(CH₂)_{y}C(O)—NH₂, SO₂NH₂; —C(O)CH₃;
   wherein y is 0 or an integer from 1 to 5; or
      a pharmaceutically acceptable salt thereof.

In another aspect, the present invention, relates to a compound of Formula (VIIIB):

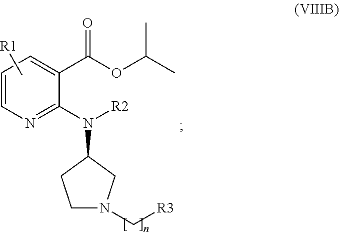

wherein:
n is 1

### R₁ is H;

R₂ is C₁₋₆ alkyl, cycloalkyl or (CH₂)ₓ-cycloalkyl;

wherein:

x is 0 or an integer from 1 to 5;

R₃ is C₁₋₆ alkyl, alkoxyalkyl, phenyl, heteroaryl;

wherein R₃ optionally is substituted with at least one of following substitutents: straight or branched C₁₋₆ alkyl, straight or branched C₁₋₆ haloalkyl, straight or branched C₁₋₆-alkoxy, straight or branched C₁₋₆-halosubstituted alkoxy, phenyl. phenoxy, benzyloxy, 3-pyridinyl or 2-thienyl;
wherein phenoxy or benzyloxy optionally is substituted by at least one of following substituents: halogen, —CN, straight or branched C₁₋₆ alkyl, straight or branched C₁₋₆-alkoxy, —O(CH₂)_{y}C(O)—NH₂, SO₂NH₂; —C(O)CH₃;
wherein:
y is 0 or an integer from 1 to 5; or
   a pharmaceutically acceptable salt thereof.

In another aspect, the present invention relates to a compound of formula (VIII), where R₂ is methyl, R₃ is phenyl, and halogen is selected from chloro or fluoro.

In another aspect, representative a compounds of Formula (VIII), which may include, but are not limited to:
1-methylethyl 2-{methyl[(3R)-1-({2-[(trifluoromethyl)oxy]phenyl}methyl)-3-pyrrolidinyl]amino}-3-pyridinecarboxylate;
1-methylethyl 2-[[(3R)-1-({2-[(3-chlorophenyl)oxy]phenyl}methyl)-3-pyrrolidinyl](methyl)amino]-3-pyridinecarboxylate;
1-methylethyl 2-[{(3R)-1-[(2-{[4-(aminosulfonyl)phenyl]oxy}phenyl)methyl]-3-pyrrolidinyl}(methyl)amino]-3-pyridinecarboxylate;
1-methylethyl 2-{methyl[(3R)-1-({3-[(trifluoromethyl)oxy]phenyl}methyl)-3-pyrrolidinyl]amino}-3-pyridinecarboxylate;
1-methylethyl 2-{methyl[(3R)-1-({3-[(phenylmethyl)oxy]phenyl}methyl)-3-pyrrolidinyl]amino}-3-pyridinecarboxylate;
1-methylethyl 2-{methyl[(3R)-1-({3-[(1,1,2,2-tetrafluoroethyl)oxy]phenyl}methyl)-3-pyrrolidinyl]amino}-3-pyridinecarboxylate;
1-methylethyl 2-[[(3R)-1-({3-[(3,5-dichlorophenyl)oxy]phenyl}methyl)-3-pyrrolidinyl](methyl)amino]-3-pyridinecarboxylate;
1-methylethyl 2-[((3R)-1-{[4-(ethyloxy)phenyl]methyl}-3-pyrrolidinyl)(methyl)amino]-3-pyridinecarboxylate;
1-methylethyl 2-[methyl((3R)-1-{[4-(phenyloxy)phenyl]methyl}-3-pyrrolidinyl)amino]-3-pyridinecarboxylate;
1-methylethyl 2-{methyl[(3R)-1-({4-[(trifluoromethyl)oxy]phenyl}methyl)-3-pyrrolidinyl]amino}-3-pyridinecarboxylate;
1-methylethyl 2-(methyl{(3R)-1-[(4-{[(2-methylphenyl)methyl]oxy}phenyl)methyl]-3-pyrrolidinyl}amino)-3-pyridinecarboxylate;
1-methylethyl 2-[[(3R)-1-({4-[(2-amino-2-oxoethyl)oxy]phenyl}methyl)-3-pyrrolidinyl](methyl)amino]-3-pyridinecarboxylate;
1-methyl ethyl 2-{methyl[(3R)-1-({4-[({4-[(methyloxy)carbonyl]phenyl}methyl)oxy]phenyl}methyl)-3-pyrrolidinyl]amino}-3-pyridinecarboxylate;
1-methylethyl 2-[methyl((3R)-1-{[4-(3-pyridinyl)phenyl]methyl}-3-pyrrolidinyl)amino]-3-pyridinecarboxylate;
1-methylethyl 2-[methyl((3R)-1-{[2′-(methyloxy)-4-biphenylyl]methyl}-3-pyrrolidinyl)amino]-3-pyridinecarboxylate;
1-methylethyl 2-[methyl((3R)-1-{[4-(2-thienyl)phenyl]methyl}-3-pyrrolidinyl)amino]-3-pyridinecarboxylate;
1-methylethyl 2-{methyl[(3R)-1-({2-[(phenylmethyl)oxy]phenyl}methyl)-3-pyrrolidinyl]amino}-3-pyridinecarboxylate;
1-methylethyl 2-[[(3R)-1-(4-biphenylylmethyl)-3-pyrrolidinyl](methyl)amino]-3-pyridinecarboxylate;
1-methylethyl 2-[{(3R)-1-[(4′-fluoro-3-biphenylyl)methyl]-3-pyrrolidinyl}(methyl)amino]-3-pyridinecarboxylate;
1-methylethyl 2-(methyl{(3R)-1-[(Z-methyl-3-biphenylyl)methyl]-3-pyrrolidinyl}amino)-3-pyridinecarboxylate;
1-methylethyl 2-[{(3R)-1-[(4′-fluoro-2-biphenylyl)methyl]-3-pyrrolidinyl}(methyl)amino]-3-pyridinecarboxylate;
1-methylethyl 2-(methyl{(3R)-1-[(2-methyl-2-biphenylyl)methyl]-3-pyrrolidinyl}amino)-3-pyridinecarboxylate;
1-methylethyl 2-[methyl((3R)-1-{[3-(phenyloxy)phenyl]methyl}-3-pyrrolidinyl)amino]-3-pyridinecarboxylate;
1-methylethyl 2-[methyl((3R)-1-{[3-(propyloxy)phenyl]methyl}-3-pyrrolidinyl)amino]-3-pyridinecarboxylate;
1-methylethyl 2-[methyl((3R)-1-{[4-(propyloxy)phenyl]methyl}-3-pyrrolidinyl)amino]-3-pyridinecarboxylate; or
   a pharmaceutically acceptable salt thereof.

The present invention also relates to a compound of formula (IX):

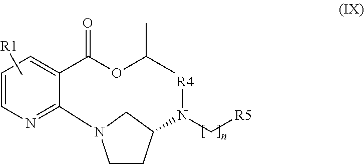

wherein:
n is 1;

### R₁ is H;

R₄ is C₁₋₆ alkyl, cycloalkyl or (CH₂)ₓ-cycloalkyl;
R₅ is C₁₋₆ alkyl, alkoxyalkyl, phenyl or heteroaryl;

wherein R₅ is optionally substituted with at least one of the following substitutents: phenyl, phenoxy, 3-pyridinyl or 2-thienyl;
wherein phenyl, phenoxy, pyridinyl or thienyl is optionally substituted by at least one of the following substituents: halogen, straight or branched C₁₋₆ alkyl, straight or branched C₁₋₆-alkoxy; or
   a pharmaceutically acceptable salt thereof.

In another aspect, the present invention relates to a compound of Formula (IX), where R₄ is ethyl; R₅ is phenyl or furanyl; R₄ is C₁₋₆ alkyl, cycloalkyl or (CH₂)ₓ-cycloalkyl and R₅ is C₁₋₆ alkyl, alkoxyalkyl, phenyl, heteroaryl.

Representative compounds of Formula (IX), which may include, but are not limited to:
1-methylethyl-2-{(3R)-3-[(3-biphenylylmethyl)(ethyl)amino]-1-pyrrolidinyl}-3-pyridine carboxylate;
1-methylethyl 2-[(3R)-3-(ethyl{[4′-(methyloxy)-4-biphenylyl]methyl}amino)-1-pyrrolidinyl]-3-pyridinecarboxylate;
1-methylethyl 2-{(3R)-3-[{[5-(2-chlorophenyl)-2-furanyl]methyl}(ethyl)amino]-1-pyrrolidinyl}-3-pyridinecarboxylate;
1-methylethyl 2-((3R)-3-{ethyl[(5-phenyl-2-furanyl)methyl]amino}-1-pyrrolidinyl)-3-pyridinecarboxylate;
1-methylethyl 2-{(3R)-3-[(4-biphenylylmethyl)(ethyl)amino]-1-pyrrolidinyl}-3-pyridinecarboxylate;
1-methylethyl 2-[(3R)-3-(ethyl{[4′-(3-pyridinyl)phenyl]methyl}amino)-1-pyrrolidinyl]-3-pyridinecarboxylate;
1-methylethyl 2-[(3R)-3-(ethyl{[4-(2-thienyl)phenyl]methyl}amino)-1-pyrrolidinyl]-3-pyridinecarboxylate;
1-methylethyl 2-[(3R)-3-(ethyl{[3-(phenyloxy)phenyl]methyl}amino)-1-pyrrolidinyl]-3-pyridinecarboxylate; or
   a pharmaceutically acceptable salt thereof.

In another aspect, the present invention relates to a compound of formula (X):

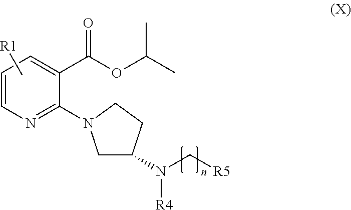

wherein:
n is 1;

### R₁ is H;

R₄ is ethyl;
R₅ is phenyl;
R₄ is C₁₋₆ alkyl, cycloalkyl or (CH₂)ₓ-cycloalkyl;
R₅ is C₁₋₆ alkyl, alkoxyalkyl, phenyl, heteroaryl;

wherein R₅ is optionally substituted with at least one of the following substitutents: straight or branched C₁₋₆ alkyl, straight or branched C₁₋₆-alkoxy, phenoxy or benzyloxy;
wherein phenoxy or benzyloxy is optionally substituted by at least one of the following substituents: halogen, straight or branched C₁₋₆ alkyl, straight or branched C₁₋₆-alkoxy; or
   a pharmaceutically acceptable salt thereof.

In another aspect, the present invention relates to a compound of Formula (X), where R₄ is ethyl and R₅ is phenyl or furanyl.

Representative examples of Formula (X), which may include, but are not limited to::
1-methylethyl 2-{(3S)-3-[ethyl({4-(methyloxy)-3-[(phenylmethyl)oxy]phenyl}methyl)amino]-1-pyrrolidinyl}-3-pyridine carboxylate;
1-methylethyl 2-{(3S)-3-[ethyl({4-[(4-fluorophenyl)oxy]phenyl}methyl)amino]-1-pyrrolidinyl}-3-pyridinecarboxylate;
1-methylethyl 2-{(3S)-3-[{[4-{[(2-chloro-6-fluorophenyl)methyl]oxy}-3-(methyloxy)phenyl]methyl}(ethyl)amino]-1-pyrrolidinyl}-3-pyridinecarboxylate;
1-methylethyl 2-[(3S)-3-(ethyl{[4-{[(4-fluorophenyl)methyl]oxy}-3-(methyloxy)phenyl]methyl)amino]-1-pyrrolidinyl}-3-pyridinecarboxylate;
1-methylethyl 2-{(3S)-3-[ethyl({3-(methyloxy)-2-[(phenylmethyl)oxy]phenyl}methyl)amino]-1-pyrrolidinyl}-3-pyridinecarboxylate;
1-methylethyl 2-{(3S)-3-[ethyl({4-[(phenylmethyl)oxy]phenyl}methyl)amino]-1-pyrrolidinyl}-3-pyridinecarboxylate;
1-methylethyl 2-{(3S)-3-[ethyl({2-[(4-fluorophenyl)oxy]phenyl}methyl)amino]-1-pyrrolidinyl}-3-pyridinecarboxylate;
1-methylethyl 2-[(3S)-3-(ethyl{[2-(phenyloxy)phenyl]methyl}amino)-1-pyrrolidinyl]-3-pyridinecarboxylate;
1-methylethyl 2-[(3S)-3-(ethyl{[4-(phenyloxy)phenyl]methyl}amino)-1-pyrrolidinyl]-3-pyridinecarboxylate;
1-methylethyl 2-{(3S)-3-[ethyl({3-[(phenylmethyl)oxy]phenyl}methyl)amino]-1-pyrrolidinyl}-3-pyridinecarboxylate;
1-methylethyl 2-{(3S)-3-[ethyl({3-(methyloxy)-4-[(phenylmethyl)oxy]phenyl}methyl)amino]-1-pyrrolidinyl}-3-pyridinecarboxylate;
1-methylethyl 2-{(3S)-3-[ethyl({2-(methyloxy)-4-[(phenylmethyl)oxy]phenyl}methyl)amino]-1-pyrrolidinyl}-3-pyridinecarboxylate;
1-methylethyl 2-{(3S)-3-[ethyl({2-[(phenylmethyl)oxy]phenyl}methyl)amino]-1-pyrrolidinyl}-3-pyridinecarboxylate;
1-methylethyl 2-[(3S)-3-(ethyl{[3-(phenyloxy)phenyl]methyl}amino)-1-pyrrolidinyl]-3-pyridinecarboxylate;
1-methylethyl 2-((3S)-3-{ethyl[(2-{[4-(methyloxy)phenyl]oxy}phenyl)methyl]amino}-1-pyrrolidinyl)-3-pyridinecarboxylate;
1-methylethyl 2-{(3S)-3-{[4-[(4-cyanophenyl)oxy]phenyl}methyl)(ethyl)amino]-1-pyrrolidinyl}-3-pyridinecarboxylate;
1-methylethyl 2-((3S)-3-{ethyl[(4-{[4-(methyloxy)phenyl]oxy}phenyl)methyl]amino}-1-pyrrolidinyl)-3-pyridinecarboxylate;
1-methylethyl 2-((3S)-3-{ethyl[(3-{[4-(methyloxy)phenyl]oxy}phenyl)methyl]amino}-1-pyrrolidinyl)-3-pyridinecarboxylate; or
   a pharmaceutically acceptable salt thereof.

In another aspect, the present invention relates to a compound of formula (XI):

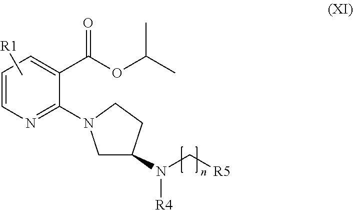

wherein:
n is 1;
R₁ is H, methyl or phenyl;
R₄ is straight or branched C₁₋₆ alkyl, cycloalkyl or (CH₂)ₓ-cycloalkyl;
R₅ is straight or branched C₁₋₆ alkyl, alkoxyalkyl, phenyl, heteroaryl;

wherein R₅ optionally is substituted with at least one of the following substitutents straight or branched C₁₋₆ alkyl, straight or branched C₁₋₆ haloalkyl, straight or branched C₁₋₆-alkoxy, —O(CH₂)ₙC(O)Rₓ, phenyl, substituted phenyl, phenoxy, benzyloxy, pyridinyl, thienyl, piperidinyl or —(CH₂)ₓ—N(R₁ₕ) —(CH₂)ₓR₁ₗ;

wherein:
R₁ₕ is H, straight or branched C₁₋₆ alkyl;
R₁ᵢ is phenyl or substituted phenyl;
x as defined for substituents defined above is 0 or an integer from 1 to 5,
wherein:
   each phenyl or substituted phenyl substitutent as defined in R₁ᵢ, above further is optionally substituted by one or more of following

substituents selected from: —H, —OH, —CN, —NO₂,-halogen, —(CH₂)_{y}—OH, —OC(O)OH, —OC(O)R₁ⱼ, —C(O)OR₁ₖ, —SO₂N(R₁ₗ)₂, -straight or branched C₁₋₆ alkyl,-straight or branched C₁₋₆ haloalkyl, -straight or branched C₁₋₆ straight or branched alkoxy; or
Rₓ is straight or branched C₁₋₆ alkyl benzyloxy, phenoxy, substituted phenyl is optionally substituted by at least one of the following substituents halogen, —CN, straight or branched C₁₋₆ alkyl straight or branched C₁₋₆-alkoxy;
   wherein:
   y is 0 or an integer from 1 to 5;
   R₁ⱼ, R₁ₖ or R₁ₗ is H, straight or branched C₁₋₆ alkyl, phenyl or substituted phenyl; or
      a pharmaceutically acceptable salt thereof.

The present invention also relates to a compound of formula (XI), where R₄ is ethyl and R₅ is phenyl, furanyl, thienyl, piperidinyl, or pyridinyl.

Representative examples of compounds of Formula (XI), include, but are not limited to:
1-methylethyl 2-{(3R)-3-[({2-[(difluoromethyl)oxy]phenyl}methyl)(ethyl)amino]-1-pyrrolidinyl}-3-pyridinecarboxylate;
1-methylethyl 2-[(3R)-3-(ethyl{[2-(methyloxy)phenyl]methyl}amino)-1-pyrrolidinyl]-3-pyridinecarboxylate;
1-methylethyl 2-{(3R)-3-[ethyl({2-[(4-fluorophenyl)oxy]phenyl}methyl)amino]-1-pyrrolidinyl}-3-pyridinecarboxylate;
1-methylethyl 2-((3R)-3-{ethyl[(2-{[2-(ethyloxy)-2-oxoethyl]oxy}phenyl)methyl]amino}-1-pyrrolidinyl)-3-pyridinecarboxylate;
1-methylethyl 2-[(3R)-3-(ethyl{[3-(ethyloxy)phenyl]methyl}amino)-1-pyrrolidinyl]-3-pyridinecarboxylate;
1-methylethyl 2-{(3R)-3-[({3-[(4-chlorophenyl)oxy]phenyl}methyl)(ethyl)amino]-1-pyrrolidinyl}-3-pyridinecarboxylate;
1-methylethyl 2-{(3R)-3-[[(3-{[4-(1,1-dimethylethyl)phenyl]oxy}phenyl)methyl](ethyl)amino]-1-pyrrolidinyl}-3-pyridinecarboxylate;
1-methylethyl 2-{(3R)-3-[{[3-(butyloxy)phenyl]methyl}(ethyl)amino]-1-pyrrolidinyl}-3-pyridinecarboxylate;
1-methylethyl 2-{(3R)-3-[ethyl({4-[(phenylmethyl)oxy]phenyl}methyl)amino]-1-pyrrolidinyl}-3-pyridinecarboxylate;
1-methylethyl 2-[(3R)-3-(ethyl{[4-(methyloxy)phenyl]methyl}amino)-1-pyrrolidinyl]-3-pyridinecarboxylate;
1-methylethyl 2-{(3R)-3-[ethyl({4-[(1-methylethyl)oxy]phenyl}methyl)amino]-1-pyrrolidinyl}-3-pyridinecarboxylate;
1-methylethyl 2-[(3R)-3-(ethyl{[4-(hexyloxy)phenyl]methyl}amino)-1-pyrrolidinyl]-3-pyridinecarboxylate;
1-methylethyl 2-((3R)-3-{ethyl[(4-{[(4-fluorophenyl)methyl]oxy}phenyl)methyl]amino}-1-pyrrolidinyl)-3-pyridinecarboxylate;
1-methylethyl 2-{(3R)-3-[ethyl({4-[(2-methylpropyl)oxy]phenyl}methyl)amino]-1-pyrrolidinyl}-3-pyridinecarboxylate;
1-methylethyl 2-((3R)-3-{ethyl[(4′-ethyl-4-biphenylyl)methyl]amino}-1-pyrrolidinyl)-3-pyridinecarboxylate;
1-methylethyl 2-{(3R)-3-[[(2′-chloro-4-biphenylyl)methyl](ethyl)amino]-1-pyrrolidinyl}-3-pyridinecarboxylate;
1-methylethyl 2-{(3R)-3-[ethyl({2-[(phenylmethyl)oxy]phenyl}methyl)amino]-1-pyrrolidinyl}-3-pyridinecarboxylate;
1-methylethyl 2-[(3R)-3-(ethyl{[3-(2-pyridinyl)phenyl]methyl}amino)-1-pyrrolidinyl]-3-pyridinecarboxylate;
1-methylethyl 2-((3R)-3-{ethyl[(4′-fluoro-3-biphenylyl)methyl]amino}-1-pyrrolidinyl)-3-pyridinecarboxylate;
1-methylethyl 2-[(3R)-3-(ethyl{[2-(3-pyridinyl)phenyl]methyl}amino)-1-pyrrolidinyl]-3-pyridinecarboxylate;
1-methylethyl 2-((3R)-3-{ethyl[(4′-fluoro-2-biphenylyl)methyl]amino}-1-pyrrolidinyl)-3-pyridinecarboxylate;
1-methylethyl 2-[(3R)-3-(ethyl{[3-(propyloxy)phenyl]methyl}amino)-1-pyrrolidinyl]-3-pyridinecarboxylate;
1-methylethyl 2-[(3R)-3-(ethyl{[4-(propyloxy)phenyl]methyl}amino)-1-pyrrolidinyl]-3-pyridinecarboxylate; 1-methylethyl 2-{(3R)-3-[ethyl(2-furanylmethyl)amino]-1-pyrrolidinyl}-4-phenyl-3-pyridinecarboxylate;
1-methylethyl 2-[(3R)-3-(ethyl{[2-(ethyloxy)phenyl]methyl}amino)-1-pyrrolidinyl]-4-phenyl-3-pyridinecarboxylate;
1-methylethyl 2-{(3R)-3-[ethyl(2-thienylmethyl)amino]-1-pyrrolidinyl}-4-phenyl-3-pyridinecarboxylate;
1-methylethyl 2-[(3R)-3-(ethyl{[3-(methyloxy)phenyl]methyl}amino)-1-pyrrolidinyl]-4-phenyl-3-pyridinecarboxylate;
1-methylethyl 2-{(3R)-3-[ethyl(3-furanylmethyl)amino]-1-pyrrolidinyl}-4-phenyl-3-pyridinecarboxylate;
1-methylethyl 2-((3R)-3-{ethyl[(5-methyl-2-thienyl)methyl]amino}-1-pyrrolidinyl)-4-phenyl-3-pyridinecarboxylate;
1-methylethyl 2-{(3R)-3-[ethyl({4-[(phenylmethyl)oxy]phenyl}methyl)amino]-1-pyrrolidinyl}-4-phenyl-3-pyridinecarboxylate;
1-methylethyl 2-{(3R)-3-[ethyl({4-[(methyloxy)carbonyl]phenyl}methylamino]-1-pyrrolidinyl}-4-phenyl-3-pyridinecarboxylate;
1-methylethyl 2-[(3R)-3-(ethyl{[4-(methyloxy)phenyl]methyl}amino)-1-pyrrolidinyl]-4-phenyl-3-pyridinecarboxylate;
1-methylethyl 2-[(3R)-3-(ethyl{[4-(ethyloxy)phenyl]methyl}amino)-1-pyrrolidinyl]-4-phenyl-3-pyridinecarboxylate;
1-methylethyl 2-[(3R)-3-(ethyl{[4-(propyloxy)phenyl]methyl}amino)-1-pyrrolidinyl]-4-phenyl-3-pyridinecarboxylate;
1-methylethyl 2-{(3R)-3-[ethyl({2-[(trifluoromethyl)oxy]phenyl}methyl)amino]-1-pyrrolidinyl}-4-phenyl-3-pyridinecarboxylate;
1-methylethyl 2-((3R)-3-{ethyl[(2-methylphenyl)methyl]amino}-1-pyrrolidinyl)-4-phenyl-3-pyridinecarboxylate;
1-methylethyl 2-((3R)-3-{ethyl[(3-fluorophenyl)methyl]amino}-1-pyrrolidinyl)-4-phenyl-3-pyridinecarboxylate;
1-methylethyl 2-{(3R)-3-[ethyl({4-(methyloxy)-3-[(phenylmethyl)oxy]phenyl}methyl)amino]-1-pyrrolidinyl}-4-phenyl-3-pyridinecarboxylate;
1-methylethyl 2-((3R)-3-{ethyl[(3-fluoro-2-methylphenyl)methyl]amino}-1-pyrrolidinyl)-4-phenyl-3-pyridinecarboxylate;
1-methylethyl 2-{(3R)-3-[ethyl({2-[(1-methylethyl)oxy]phenyl}methyl)amino]-1-pyrrolidinyl}-4-phenyl-3-pyridinecarboxylate;
1-methylethyl 2-[(3R)-3-(ethyl{[4-(3-pyridinyl)phenyl]methyl}amino)-1-pyrrolidinyl]-4-phenyl-3-pyridinecarboxylate;
1-methylethyl 2-{(3R)-3-[ethyl({3-[(1-methylethyl)oxy]phenyl}methyl)amino]-1-pyrrolidinyl}-4-phenyl-3-pyridinecarboxylate;
1-methylethyl 2-((3R)-3-{ethyl[(5-ethyl-2-thienyl)methyl]amino}-1-pyrrolidinyl)-4-phenyl-3-pyridinecarboxylate;
1-methylethyl 2-[(3R)-3-(ethyl{[3-(ethyloxy)phenyl]methyl}amino)-1-pyrrolidinyl]-4-phenyl-3-pyridinecarboxylate;
1-methylethyl 2-{(3R)-3-[ethyl({4-[(2-methylpropyl)oxy]phenyl}methyl)amino]-1-pyrrolidinyl}-4-methyl-3-pyridinecarboxylate;
1-methylethyl 2-{(3R)-3-[ethyl({2-[(phenylmethyl)oxy]phenyl}methyl)amino]-1-pyrrolidinyl}-4-methyl-3-pyridinecarboxylate;
1-methylethyl 2-[(3R)-3-(ethyl{[3-(phenyloxy)phenyl]methyl}amino)-1-pyrrolidinyl]-4-methyl-3-pyridinecarboxylate;
1-methylethyl 2-[(3R)-3-(ethyl{[4-(propyloxy)phenyl]methyl}amino)-1-pyrrolidinyl]-4-methyl-3-pyridinecarboxylate;
1-methylethyl 2-{(3R)-3-[ethyl(3-pyridinylmethyl)amino]-1-pyrrolidinyl}-4-methyl-3-pyridinecarboxylate;
1-methylethyl 2-{(3R)-3-[ethyl(3-furanylmethyl)amino]-1-pyrrolidinyl}-4-methyl-3-pyridinecarboxylate;
1-methylethyl 2-((3R)-3-{ethyl[(5-methyl-2-thienyl)methyl]amino}-1-pyrrolidinyl)-4-methyl-3-pyridinecarboxylate;
1-methylethyl 2-[(3R)-3-(ethyl{[2-(3-pyridinyl)phenyl]methyl}amino)-1-pyrrolidinyl]-4-methyl-3-pyridinecarboxylate;
1-methylethyl 2-{(3R)-3-[[1-(3-chlorophenyl)-4-piperidinyl](ethyl)amino]-1-pyrrolidinyl}-4-methyl-3-pyridinecarboxylate;
1-methylethyl 2-((3R)-3-{ethyl[(4′-fluoro-3-biphenylyl)methyl]amino}-1-pyrrolidinyl)-4-methyl-3-pyridinecarboxylate;
1-methylethyl 2-((3R)-3-{ethyl[(2′-methyl-2-biphenylyl)methyl]amino}-1-pyrrolidinyl)-4-methyl-3-pyridinecarboxylate;
1-methylethyl 2-[(3R)-3-(ethyl{[2-(ethyloxy)phenyl]methyl}amino)-1-pyrrolidinyl]-4-methyl-3-pyridinecarboxylate;
1-methylethyl 2-[(3R)-3-(ethyl{[2-(phenyloxy)phenyl]methyl}amino)-1-pyrrolidinyl]-4-methyl-3-pyridinecarboxylate;
1-methylethyl 2-{(3R)-3-[({2-[(3-chlorophenyl)oxy]phenyl}methyl)(ethyl)amino]-1-pyrrolidinyl}-4-methyl-3-pyridinecarboxylate;
1-methylethyl 2-[(3R)-3-(ethyl{[2-(propyloxy)phenyl]methyl}amino)-1-pyrrolidinyl]-4-methyl-3-pyridinecarboxylate;
1-methylethyl 2-[(3R)-3-(ethyl{[3-(methyloxy)phenyl]methyl}amino)-1-pyrrolidinyl]-4-methyl-3-pyridinecarboxylate;
1-methylethyl 2-{(3R)-3-[({3-[(4-chlorophenyl)oxy]phenyl}methyl)(ethyl)amino]-1-pyrrolidinyl}-4-methyl-3-pyridinecarboxylate;
1-methylethyl 2-{(3R)-3-[ethyl({3-[(2-methylpropyl)oxy]phenyl}methyl)amino]-1-pyrrolidinyl}-4-methyl-3-pyridinecarboxylate;
1-methylethyl 2-{(3R)-3-[ethyl({4-[(phenylmethyl)oxy]phenyl}methyl)amino]-1-pyrrolidinyl}-4-methyl-3-pyridinecarboxylate;
1-methylethyl 2-[(3R)-3-(ethyl{[4-(methyloxy)phenyl]methyl}amino)-1-pyrrolidinyl]-4-methyl-3-pyridinecarboxylate;
1-methylethyl 2-{(3R)-3-[[(4,5-dimethyl-2-furanyl)methyl](ethyl)amino]-1-pyrrolidinyl}-4-methyl-3-pyridinecarboxylate;
1-methylethyl 2-{(3R)-3-[ethyl(phenylmethyl)amino]-1-pyrrolidinyl}-4-methyl-3-pyridinecarboxylate;
1-methylethyl 2-{(3R)-3-[ethyl({4-[(1-methylethyl)oxy]phenyl}methyl)amino]-1-pyrrolidinyl}-4-methyl-3-pyridinecarboxylate;
1-Methylethyl-2-((3R)-3-{ethyl[(4′-fluoro-2-biphenylyl)methyl]amino}-1-pyrrolidinyl)-3-pyridinecarboxylate;
1-methylethyl 2-{(3R)-3-[[(4-{[[(2-chloro-6-fluorophenyl)methyl](ethyl)amino]methyl}phenyl)methyl] (ethyl)amino]-1-pyrrolidinyl}-3-pyridinecarboxylate;
1-methylethyl 2-{(3R)-3-[[(3-{[[(2-chloro-6-fluorophenyl)methyl](ethyl)amino]methyl}phenyl)methyl]ethyl)amino]-1-pyrrolidinyl}-3-pyridine carboxylate; or
   a pharmaceutically acceptable salt thereof.

In another aspect, additional representative compounds, which are encompassed and defined by Formulas (I) to (XI), respectively of the present invention, include, but are not limited to:
1-Methylethyl2-{4-[(5-ethyl-2-thienyl)methyl]-1-piperazinyl}-3-pyridinecarboxylate;
1-Methylethyl 2-{4-[(4,5-dimethyl-2-thienyl)methyl]-1-piperazinyl}-3-pyridine carboxylate;
1-Methylethyl 2-{4-[(4-ethylphenyl)methyl]-1-piperazinyl}-3-pyridinecarboxylate;
1-Methylethyl 2-{4-[(2-ethylphenyl)methyl]-1-piperazinyl}-3-pyridinecarboxylate; 1-Methylethyl 2-{methyl[(3S)-1-(phenylmethyl)-3-pyrrolidinyl]amino}-3-pyridine carboxylate hydrochloride;
1-Methylethyl 2-{methyl[(3R)-1-(phenylmethyl)-3-pyrrolidinyl]amino}-3-pyridine carboxylate hydrochloride;
1-Methylethyl 2-((3S)-3-{[(5-ethyl-2-thienyl)methyl]amino}-1-pyrrolidinyl)-3-pyridinecarboxylate;
1-Methylethyl 2-((3S)-3-{[(4,5-dimethyl-2-thienyl)methyl]amino}-1-pyrrolidinyl)-3-pyridinecarboxylate;
1-Methylethyl 2-((3S)-3-{[(3-ethylphenyl)methyl]amino}-1-pyrrolidinyl)-3-pyridinecarboxylate; 1-Methylethyl 2-((3S)-3-{[(4-ethylphenyl)methyl]amino}-1-pyrrolidinyl)-3-pyridinecarboxylate;
1-Methylethyl 2-((3S)-3-{[(2-ethylphenyl)methyl]amino}-1-pyrrolidinyl)-3-pyridinecarboxylate;
1-Methylethyl 2-{(3S)-3-[[(5-ethyl-2-thienyl)methyl](methyl)amino]-1-pyrrolidinyl}-3-pyridinecarboxylate;
1-Methylethyl 2-{(3S)-3-[[(4,5-dimethyl-2-thienyl)methyl](methyl)amino]-1-pyrrolidinyl}-3-pyridinecarboxylate;
1-Methylethyl 2-{(3S)-3-[[(3-ethylphenyl)methyl](methyl)amino]-1-pyrrolidinyl}-3-pyridinecarboxylate;
1-Methylethyl 2-{(3S)-3-[[(4-ethylphenyl)methyl](methyl)amino]-1-pyrrolidinyl}-3-pyridinecarboxylate;
1-Methylethyl 2-{(3S)-3-[[(2-ethylphenyl)methyl](methyl)amino]-1-pyrrolidinyl}-3-pyridinecarboxylate;
1-Methylethyl 2-((3S)-3-{ethyl[(5-ethyl-2-thienyl)methyl]amino}-1-pyrrolidinyl)-3-pyridinecarboxylate;
1-Methylethyl 2-{(3S)-3-[[(4,5-dimethyl-2-thienyl)methyl](ethyl)amino]-1-pyrrolidinyl}-3-pyridinecarboxylate;
1-Methylethyl 2-((3S)-3-{ethyl[(3-ethylphenyl)methyl]amino}-1-pyrrolidinyl)-3-pyridinecarboxylate;
1-Methylethyl 2-((3S)-3-{ethyl[(4-ethylphenyl)methyl]amino}-1-pyrrolidinyl)-3-pyridinecarboxylate;
1-Methylethyl 2-((3S)-3-{ethyl[(2-ethylphenyl)methyl]amino}-1-pyrrolidinyl)-3-pyridinecarboxylate;
1-Methylethyl 2-(4-{[3-({[3-(methyloxy)phenyl]methyl}oxy)phenyl]methyl}-1-piperazinyl)-3-pyridinecarboxylate hydrochloride;
1-Methylethyl 2-{(3R)-3-[ethyl(phenylmethyl)amino]-1-pyrrolidinyl}-3-pyridinecarboxylate;
2-{(3R)-3-[Ethyl(phenylmethyl)amino]-1-pyrrolidinyl}-3-pyridinecarboxylic acid;
1-Methylethyl2-{methyl[(3R)-3-pyrrolidinyl}amino]-3-pyridinecarboxylate hydrochloride;
1-methylethyl 2-(4-{[3-(Phenylmethyl)phenyl]methyl}-1-piperazinyl)-3-pyridine carboxylate hydrochloride;
1-Methylethyl 2-(4-{[4-(phenylmethyl)phenyl]methyl}-1-piperazinyl)-3-pyridine carboxylate hydrochloride;
1-Methylethyl2-[4-(2-phenylethyl)-1-piperazinyl]-3-pyridinecarboxylate hydrochloride;
1-Methylethyl 2-(4-{[4-(3-phenylpropyl)phenyl]methyl}-1-piperazinyl)-3-pyridine carboxylate hydrochloride;
1-Methylethyl2-[4-({3-[methyl(phenylcarbonyl)amino]phenyl}methyl)-1-piperazinyl]-3-pyridinecarboxylate hydrochloride;
1-Methylethyl 2-[4-({-4-[methyl(phenylcarbonyl)amino]phenyl}methyl)-1-piperazinyl]-3-pyridinecarboxylate hydrochloride;
1-methylethyl 2-[4-({3-[(dimethylamino)carbonyl]phenyl}methyl)-1-piperazinyl]-3-pyridinecarboxylate hydrochloride;
1-Methylethyl 2-[4-({4-[(dimethylamino)carbonyl]phenyl}methyl)-1-piperazinyl]-3-pyridinecarboxylate hydrochloride;
1-Methylethyl 2-(4-{[4-(hydroxymethyl)phenyl]methyl}-1-piperazinyl)-3-pyridine carboxylate hydrochloride;
1-Methylethyl 2-(4-{[3-(phenylthio)phenyl]methyl}-1-piperazinyl)-3-pyridine carboxylate hydrochloride;
1-Methylethyl 2-(4-{[4-(phenylthio)phenyl]methyl}-1-piperazinyl)-3-pyridine carboxylate hydrochloride;
1-Methylethyl 2-[4-({3-[(phenylmethyl)thio]phenyl}methyl)-1-piperazinyl]-3-pyridine carboxylate hydrochloride;
1-Methylethyl 2-[4-({4-[(phenylmethyl)thio]phenyl}methyl)-1-piperazinyl]-3-pyridine carboxylate hydrochloride;
1-Methylethyl 2-(4-{[3-(hydroxymethyl)phenyl]methyl}-1-piperazinyl)-3-pyridine carboxylate;
1-Methylethyl 2-[(3R)-3-(ethyl{[4-({ethyl[(3R)-1-(2-methylpropanoyl)-3-pyrrolidinyl]amino}methyl)phenyl]methyl}amino)-1-pyrrolidinyl]-3-pyridinecarboxylate; or
   a pharmaceutically acceptable salt thereof.

In another aspect, additional representative compounds, which are encompassed and defined by Formulas (I) to (XI), respectively of the present invention are:
1-methylethyl 2-{4-[(4-{[[(2-chloro-6-fluorophenyl)methyl](ethyl)amino]methyl}phenyl)methyl]-1-piperazinyl}-3-pyridinecarboxylate;
bis(1-methylethyl) 2,2′-(benzene-1,4-diylbis[methanediyl(ethylimino)(3S)-3,1-pyrrolidinediyl]di(3-pyridinecarboxylate);
benzene-1,4-diylbis[methanediyl(ethylimino)(3R)-3,1-pyrrolidinediyl-2,3-pyridinediylmethanediyl]bis(3,3-dimethylbutanoate;
1-methylethyl 2-{4-[(3-{[(2-chloro-6-fluorophenyl)methyl]oxy}phenyl)methyl]-1-piperazinyl}-3-pyridinecarboxylate;
1-methylethyl 2-{4-[(3-{[4-(methyloxy)phenyl]oxy}phenyl)methyl]-1-piperazinyl}-3-pyridinecarboxylate;
1-methylethyl 2-(4-{[Z-(trifluoromethyl)-3-biphenylyl]methyl}-1-piperazinyl)-3-pyridinecarboxylate;
1-methylethyl 2-(4-{[3-({[2-(methyloxy)phenyl]methyl}oxy)phenyl]methyl}-1-piperazinyl)-3-pyridinecarboxylate;
1-[(4-{[[(2-Chloro-6-fluorophenyl)methyl](ethyl)ammonio]methyl}phenyl)methyl]-4-(3-{[(1-methylethyl)oxy]carbonyl}-2-pyridinyl)piperazin-1-ium di-maleate
1-methylethyl 2-(4-{[3-({[4-(ethyloxy)phenyl]methyl}oxy)phenyl]methyl}-1-piperazinyl)-3-pyridinecarboxylate;
1-methylethyl 2-[(3R)-3-(ethyl{[4-({ethyl[(3R)-1-(1-{2-[(1-methylethyl)oxy]-2-oxoethyl]ethenyl)-3-pyrrolidinyl]amino}methyl)phenyl}methyl}amino)-1-pyrrolidinyl]-3-pyridinecarboxylate;
1-methylethyl 2-(4-{[3-({[3-(methyloxy)phenyl]methyl}oxy)phenyl]methyl}-1-piperazinyl)-3-pyridinecarboxylate;
1-methylethyl 2-{(3R)-3-[ethyl({2-[(phenylmethyl)oxy]phenyl}methyl)amino]-1-pyrrolidinyl}-3-pyridinecarboxylate;
bis(1-methylethyl) 2,2′-{benzene-1,4-diylbis[methanediyl(2S)-1,2-pyrrolidinediylmethanediyloxy]}di(3-pyridinecarboxylate); or
   pharmaceutically acceptable salts thereof.

B. Dimer Compounds

In general, the present invention relates to dimer compounds and corresponding dimer preparation methods, where the aforementioned dimers are formed from precursors, intermediates or monomeric compounds of Formulas (I) to (XI), respectively, of the present invention as defined above and a reactant containing a linker group A.

In another aspect, dimer compounds of the present invention may be structurally symmetric or asymmetric as formed based upon selection of corresponding precursors, intermediates or monomeric compounds of Formulas (I) to (XI), respectively, as defined in the present specification above.

In one aspect, as suitable for use in the present invention reactant containing a linker group A may include, but is not limited to the following functional groups straight or branched C₁-C₆-alkyl, straight or branched C₁-C₆-thioalkyl, straight or branched C₁-C₆-aminoalkyl, substituted straight or branched C₁-C₆-aminoalkyl straight or branched C₁-C₆-alkoxy, C₄-C₇cycloalkyl, aryl, heterocycloalkyl or heteroaryl as defined above in the section entitled Substituents.

In one aspect, the present invention relates to a dimer compound of formula (XII):

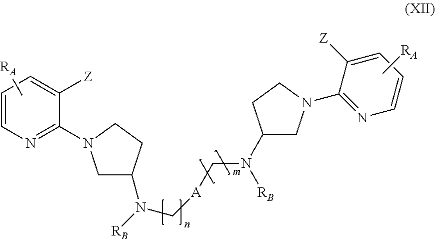

wherein:
n is 0 or an integer from 1 to 5;
m is 0 or an integer from 1 to 5;
A is straight or branched C₁₋₆ alkyl, aryl or heteroaryl;

### Z is

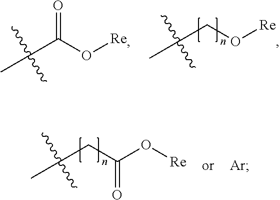

R_{A} is H, halogen, straight or branched C₁₋₆ alkyl, phenyl, substituted phenyl, —NHRₐ, —SRₐ or —ORₐ;
R_{B} is H, straight or branched C₁₋₆alkyl or cycloalkyl;

wherein:
Rₐ is selected from phenyl or substituted phenyl;
Rₑ is H, straight or branched C₁₋₆ alkyl or cycloalkyl; or
   a pharmaceutically acceptable salt thereof.

The present invention also relates to a dimer compound of Formula (XII), where A is isopropyl, dimethylpentyl or phenyl.

In another aspect, the present invention relates to a dimer compound of Formula (XIII):

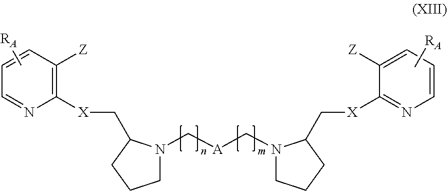

wherein:
n is 0 or an integer from 1 to 5;
m is 0 or an integer from 1 to 5;
A is straight or branched C₁₋₆ alkyl, phenyl or heteroaryl;

### X is O, N or S;

### Z is

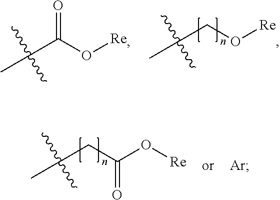

R_{A} is H, halogen, straight or branched C₁₋₆ alkyl, phenyl, substituted phenyl, —NHRₐ, —SRₐ or —ORₐ;

wherein:
Rₐ is selected from phenyl or substituted phenyl;
Rₑ is H, straight or branched C₁₋₆ alkyl or cycloalkyl; or
   a pharmaceutically acceptable salt thereof.

In another aspect, the present invention relates to a dimer compound of Formula (XIV):

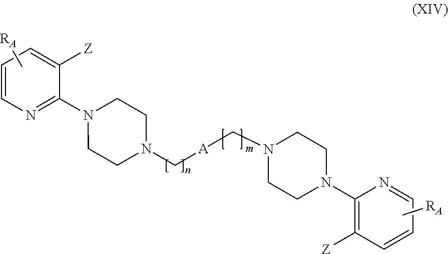

wherein:
n is 0 or an integer from 1 to 5;
m is 0 or an integer from 1 to 5;
A is straight or branched C₁₋₆ alkyl, phenyl or heteroaryl;

### Z is

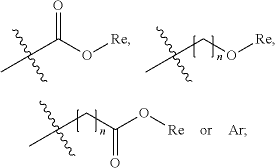

R_{A} is H, halogen, straight or branched C₁₋₆ alkyl, phenyl, substituted phenyl, —NHRₐ, —SRₐ or —ORₐ;

wherein:
Rₐ is selected from phenyl or substituted phenyl;
Rₑ is H, straight or branched C₁₋₆ alkyl or cycloalkyl; or
   a pharmaceutically acceptable salt thereof.

In another aspect, the present invention relates to a dimer compound of Formula (XV):

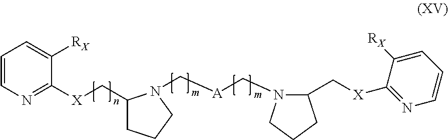

wherein:
n is 0 or an integer from 1 to 5;
m is 0 or an integer from 1 to 5;
A is straight or branched C₁₋₆ alkyl, phenyl or heteroaryl;

### X is O, N or S;

R_{K} is H, halogen, straight or branched C₁₋₆ alkyl, phenyl, substituted phenyl, —NHRₐ, —SRₐ, —ORₐ; or

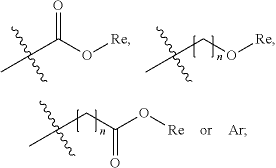

wherein:
Rₐ is selected from phenyl or substituted phenyl;
Rₑ is H, straight or branched C₁₋₆ alkyl or cycloalkyl; or
   a pharmaceutically acceptable salt thereof.

In another aspect, the present invention relates to a dimer compound of Formula (XVI):

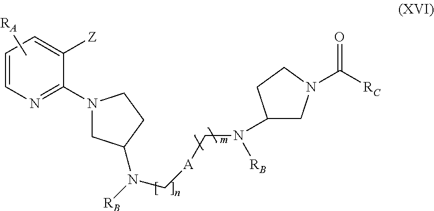

wherein:
n is 0 or an integer from 1 to 5;
m is 0 or an integer from 1 to 5;
A is straight or branched C₁₋₆ alkyl, phenyl or heteroaryl;

### Z is

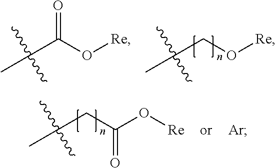

R_{A} is H, halogen, straight or branched C₁₋₆ alkyl, phenyl, substituted phenyl, —NHRₐ, —SRₐ or —ORₐ;
R_{B} is H, straight or branched C₁₋₆alkyl or cycloalkyl;
R_{C} is H, straight or branched C₁₋₆alkyl, phenyl or —OR_{b};

wherein:
Rₐ is selected from phenyl or substituted phenyl;
R_{b} is H, straight or branched C₁₋₆ alkyl or cycloalkyl;
Rₑ is H, straight or branched C₁₋₆ alkyl or cycloalkyl; or
   a pharmaceutically acceptable salt thereof.

In one aspect of the present invention, representative dimer compounds of Formulas (XII) to (XVI) are:
bis(1-methylethyl) 2,2′-{benzene-1,4-diylbis[methanediyl(ethylimino)(3R)-3,1-pyrrolidinediyl]}di(3-pyridinecarboxylate);
bis(1-methylethyl) 2,2′-{benzene-1,3-diylbis[methanediyl(ethylimino)(3R)-3,1-pyrrolidinediyl]}di(3-pyridinecarboxylate);
1-methylethyl 2-[(3R)-3-(ethyl{[4-({ethyl[(3S)-1-(3-{[(1-methylethyl)oxy]carbonyl}-2-pyridinyl)-3-pyrrolidinyl]amino}methyl)phenyl]methyl}amino)-1-pyrrolidinyl]-3-pyridinecarboxylate;
bis(1-methylethyl) 2,2′-{benzene-1,3-diylbis[methanediyl(2S)-1,2-pyrrolidinediyl methanediyloxy]}di(3-pyridinecarboxylate);
benzene-1,4-diylbis[methanediyl(ethylimino)(3R)-3,1-pyrrolidinediyl-2,3-pyridinediyl methanediyl]bis(3,3-dimethylbutanoate) hydrochloride;
benzene-1,4-diylbis[methanediyl(ethylimino)(3R)-3,1-pyrrolidinediyl-2,3-pyridinediylmethanediyl]dibenzoate hydrochloride;
bis(1-methylethyl)2,2′-[benzene-1,4-diylbis(methanediyl-4,1-piperazinediyl)]di(3-pyridinecarboxylate); bis(1-methylethyl) 2,2′-{benzene-1,4-diylbis[methanediyl(2S)-1,2-pyrrolidinediyl methanediyloxy]}di(3-pyridinecarboxylate);
1-Methylethyl 2-[(3R)-3-(ethyl{[4-({ethyl[(3R)-1-(2-methylpropanoyl)-3-pyrrolidinyl]amino}methyl)phenyl]methyl}amino)-1-pyrrolidinyl]-3-pyridinecarboxylate;
bis(1-methylethyl) 2,2′-{benzene-1,4-diylbis[methanediyl(ethylimino)(3S)-3,1-pyrrolidinediyl]}di(3-pyridinecarboxylate) or
   a pharmaceutically acceptable salt thereof.

In another aspect of the present invention, a representative dimer compound is:
bis(1-methylethyl) 2,2′-{benzene-1,4-diylbis[methanediyl(ethylimino)(3R)-3,1-pyrrolidinediyl]}di(3-pyridinecarboxylate) or a pharmaceutically acceptable salt thereof.

In another aspect of the present invention, a representive dimer compound is:
bis(1-methylethyl) 2,2′-{benzene-1,4-diylbis[methanediyl(ethylimino)(3R)-3,1-pyrrolidinediyl]}di(3-pyridinecarboxylate); or a pharmaceutically acceptable salt thereof.

In another aspect, dimer compounds of the present invention may be structurally symmetric or asymmetric as formed based upon selection of corresponding precursors, intermediates or monomeric compounds of Formulas (I) to (XVI), respectively, as defined in the present specification above.

Additional representative examples of such dimers of the present invention, include, but are not limited to:
Bis(1-methylethyl) 2,2′-{benzene-1,4-diylbis[methanediyl(ethylimino)(3R)-3,1-pyrrolidinediyl]}di(3-pyridinecarboxylate);
Bis(1-methylethyl) 2,2′-{benzene-1,3-diylbis[methanediyl(ethylimino)(3R)-3,1-pyrrolidinediyl]}di(3-pyridine carboxylate);
1-Methylethyl 2-[(3R)-3-(ethyl{[4-({ethyl[(3S)-1-(3-{[(1-methylethyl)oxy]carbonyl}-2-pyridinyl)-3-pyrrolidinyl]amino}methyl)phenyl]methyl}amino)-1-pyrrolidinyl]-3-pyridinecarboxylate;
Bis(1-methylethyl) 2,2′-{benzene-1,3-diylbis[methanediyl(2S)-1,2-pyrrolidinediyl methanediyloxy]}di(3-pyridinecarboxylate);
Benzene-1,4-diylbis[methanediyl(ethylimino)(3R)-3,1-pyrrolidinediyl-2,3-pyridinediyl methanediyl]bis(3,3-dimethylbutanoate) hydrochloride;
Benzene-1,4-diylbis[methanediyl(ethylimino)(3R)-3,1-pyrrolidinediyl-2,3-pyridinediylmethanediyl]dibenzoate hydrochloride;
bis(1-methylethyl) 2,2′-[benzene-1,4-diylbis(methanediyl-4,1-piperazinediyl)]di(3-pyridinecarboxylate);
Bis(1-methylethyl) 2,2′-{benzene-1,4-diylbis[methanediyl(2S)-1,2-pyrrolidinediyl methanediyloxy]}di(3-pyridinecarboxylate);
Bis(1-methylethyl) 2,2′-{benzene-1,4-diylbis[methanediyl(ethylimino)(3S)-3,1-pyrrolidinediyl]}di(3-pyridinecarboxylate);
Bis(1-methylethyl) 2,2′-[(ethylimino)bis(methanediylbenzene-4,1-diylmethanediyl-4,1-piperazinediyl)]di(3-pyridinecarboxylate);
(3R)—N,N-diethyl-N-{[4-({ethyl[(3R)-1-(3-{[(1-methylethyl)oxy]carbonyl}-2-pyridinyl)-3-pyrrolidinyl]amino}methyl)phenyl]methyl}-1-(3-{[(1-methylethyl)oxy]carbonyl}-2-pyridinyl)-3-pyrrolidinaminium;
1H-pyrazole-3,5-diylbis[methanediyl(ethylimino)(3R)-3,1-pyrrolidinediyl-2,3-pyridinediyl methanediyl]bis(3,3-dimethylbutanoate) quaternary hydrochloride; 2,5-pyrazinediylbis[methanediyl(ethylimino)(3R)-3,1-pyrrolidinediyl-2,3-pyridinediylmethanediyl]bis(3,3-dimethylbutanoate) hydrochloride;

Bis(1-methylethyl) 2,2′-{benzene-1,4-diylbis[methanediylimino(3R)-3,1-pyrrolidinediyl]}di(3-pyridinecarboxylate); Bis(1-methylethyl) 2,2′-[2,5-pyrazinediylbis(methanediyl-4,1-piperazinediyl)]di(3-pyridine carboxylate); or

pharmaceutically acceptable salts thereof.

It is recognized that the compounds of Formulas (I) to (XVI), respectively, of the present invention as defined above may exist in forms as stereoisomers, regioisomers, or diasteriomers. These compounds may contain one or more asymmetric carbon atoms and may exist in racemic and optically active forms. For example, compounds of the present invention may exist as a racemic mixture of R(+) and S(−) enantiomers, or in separate respectively optical forms, i.e., existing separately as either the R(+) enantiomer form or in the S(+) enantiomer form. All of these individual compounds, isomers, and mixtures thereof are included within the scope of the present invention.

### SUBSTITUENT DEFINITIONS

As used herein, the term “alkyl” represents a saturated, straight or branched hydrocarbon moiety, which may be unsubstituted or substituted by one, or more of the substituents defined herein. Exemplary alkyls include, but are not limited to methyl (Me), ethyl (Et), propyl, isopropyl, butyl, isobutyl, t-butyl, pentyl and the like. The term “C1-C-6” refers to an alkyl containing from 1 to 6 carbon atoms.

When the term “alkyl” is used in combination with other substituent groups, such as “haloalkyl” or “hydroxyalkyl”, “arylalkyl”, the term “alkyl” is intended to encompass a divalent straight or branched-chain hydrocarbon radical. For example, haloalkyl is intended to mean a saturated, straight or branched hydrocarbon moiety substituted with one or more halogen groups, where halogen is fluoro, chloro, bromo or iodo. Representative haloalkyls include, but are not limited to trifluoromethyl (—CF₃). tetrafluoroethyl (—CF₂CHF₂), pentafluoroethyl (—CF₂CF₃) and the like. For example, hydroxyalkyl is intended to mean a saturated, straight or branched hydrocarbon moiety substituted with one or more hydroxy groups.

As used herein, the term “alkenyl” refers to a straight or branched hydrocarbon moiety containing at least 1 and up to 3 carbon-carbon double bonds. Examples include ethenyl and propenyl.

As used herein, the term “alkynyl” refers to a straight or branched hydrocarbon moiety containing at least 1 and up to 3 carbon-carbon triple bonds. Examples include ethynyl and propynyl.

As used herein, the term “cycloalkyl” refers to a non-aromatic, saturated, cyclic hydrocarbon ring. The term “(C₃-C₃)cycloalkyl” refers to a non-aromatic cyclic hydrocarbon ring having from three to eight ring carbon atoms. Exemplary “(C3-C8)cycloalkyl” groups useful in the present invention include cyclopropyl, cyclobutyl, cyclopentyl, cyclohexyl, cycloheptyl, and cyclooctyl.

“Alkoxy” refers to a group containing an alkyl radical attached through an oxygen linking atom. The term “(C₁-C₆)alkoxy” refers to a straight- or branched-chain hydrocarbon radical having at least 1 and up to 6 carbon atoms attached through an oxygen linking atom. Exemplary “(C₁-C₄)-alkoxy” groups useful in the present invention include, but are not limited to, methoxy, ethoxy, n-propoxy, isopropoxy, n-butoxy, s-butoxy, and t-butoxy. Representative haloalkoxy include, but are not limited to difluoromethoxy (—OCHCF₂), trifluoromethoxy (—OCF₃), tetrafluoroethoxy (—OCF₂CHF₂) and the like.

“Alkylthio-” refers to a group containing an alkyl radical atoms attached through an sulfur linking atom. The term “(C1-C4)alkylthio-” refers to a straight- or branched-chain hydrocarbon radical having at least 1 and up to 4 carbon atoms attached through a sulfur linking atom. Exemplary “(C₁-C₄)alkylthio-” groups useful in the present invention include, but are not limited to, methylthio-, ethylthio-, n-propylthio-, isopropylthio-, n-butylthio-, s-butylthio-, t-butylthio- and the like.

“Cycloalkyloxy”, “cycloalkylthio”, “cycloalkylamino” refers to a group containing a saturated carbocyclic ring atoms attached through an oxygen, nitrogen or sulfur linking atom, respectively.

“Aryl” represents a group or moiety comprising an aromatic, monovalent monocyclic or bicyclic hydrocarbon radical containing from 6 to 10 carbon ring atoms, which may be unsubstituted or substituted by one or more of the substituents defined herein, and to which may be fused one or more cycloalkyl rings, which may be unsubstituted or substituted by one or more substituents defined herein. Representative aryl groups suitable for use in the present invention, may include, but are not limited to phenyl, naphthalenyl, fluorenyl, and the like.

Heterocyclic groups may be heteroaryl or heterocycloalkyl groups.

“Heterocycloalkyl” represents a group or moiety comprising a non-aromatic, monovalent monocyclic or bicyclic radical, which is saturated or partially unsaturated, containing 3 to 10 ring atoms, which includes 1 to 4 heteroatoms independently selected from nitrogen, oxygen and sulfur, and which may be unsubstituted or substituted by one or more of the substituents defined herein. Illustrative examples of heterocycloalkyls include, but are not limited to, azetidinyl, pyrrolidyl (or pyrrolidinyl), piperidinyl, piperazinyl, morpholinyl, tetrahydro-2H-1,4-thiazinyl, tetrahydrofuryl (or tetrahydrofuranyl), dihydrofuryl, oxazolinyl, thiazolinyl, pyrazolinyl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl, 1,5,9-triazacyclododecyl and the like.

Generally, in the compounds of this invention, heterocycloalkyl groups are 5-membered and/or 6-membered heterocycloalkyl groups, such as pyrrolidyl (or pyrrolidinyl), tetrahydrofuryl (or tetrahydrofuranyl), tetrahydrothienyl, dihydrofuryl, oxazolinyl, thiazolinyl or pyrazolinyl, piperidyl (or piperidinyl), piperazinyl, morpholinyl, tetrahydropyranyl, dihydropyranyl, 1,3-dioxanyl, tetrahydro-2H-1,4-thiazinyl, 1,4-dioxanyl, 1,3-oxathianyl, and 1,3-dithianyl.

“Heteroaryl” represents a group or moiety comprising an aromatic monovalent monocyclic or bicyclic radical, containing 5 to 10 ring atoms, including 1 to 4 heteroatoms independently selected from nitrogen, oxygen and sulfur, which may be unsubstituted or substituted by one or more of the substituents defined herein. This term also encompasses bicyclic heterocyclic-aryl compounds containing an aryl ring moiety fused to a heterocycloalkyl ring moiety, containing 5 to 10 ring atoms, including 1 to 4 heteroatoms independently selected from nitrogen, oxygen and sulfur, which may be unsubstituted or substituted by one or more of the substituents defined herein. Illustrative examples of heteroaryls include, but are not limited to, thienyl, pyrrolyl, imidazolyl, pyrazolyl, furyl (or furanyl), isothiazolyl, furazanyl, isoxazolyl, oxazolyl, oxadiazolyl, thiazolyl, pyridyl (or pyridinyl), pyrazinyl, pyrimidinyl, pyridazinyl, triazinyl, tetrazinyl, triazolyl, tetrazolyl, benzo[b]thienyl, isobenzofuryl, 2,3-dihydrobenzofuryl, chromenyl, chromanyl, indolizinyl, isoindolyl, indolyl, indazolyl, purinyl, isoquinolyl, quinolyl, phthalazinyl, naphthridinyl, quinazolinyl, benzothiazolyl, benzimidazolyl, tetrahydroquinolinyl, cinnolinyl, pteridinyl, isothiazolyl, carbazolyl, 1,2,3,4 tetrahydro isoquinolinyl and the like.

Generally, the heteroaryl groups present in the compounds of this invention are 5-membered and/or 6-membered monocyclic heteroaryl groups. Selected 5-membered heteroaryl groups contain one nitrogen, oxygen or sulfur ring heteroatom, and optionally contain 1, 2 or 3 additional nitrogen ring atoms. Selected 6-membered heteroaryl groups contain 1, 2, 3 or 4 nitrogen ring heteroatoms. Selected 5- or 6-membered heteroaryl groups include thienyl, pyrrolyl, imidazolyl, pyrazolyl, furyl, isothiazolyl, furazanyl, isoxazolyl, oxazolyl, oxadiazolyl, thiazolyl, triazolyl, and tetrazolyl or pyridyl, pyrazinyl, pyrimidinyl, pyridazinyl, and triazinyl.

“Oxo” represents a double-bonded oxygen moiety; for example, if attached directly to a carbon atom forms a carbonyl moiety (C═O), or attached to an N or S forms oxides, N-oxides, sulfones or sulfoxides.

The terms “halogen” and “halo” represent chloro, fluoro, bromo or iodo substituents. “Hydroxy” or “hydroxyl” is intended to mean the radical —OH.

As used herein, the term “compound(s) of the invention” means a compound of Formulas (I) to (XVI), respectively (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” means that a group, such as, which may include, but is not limited to alkyl, aryl, heteroaryl, etc., may be unsubstituted, or the group may be substituted with one or more substituent(s) as defined. In the case where groups may be selected from a number of alternative groups the selected groups may be the same or different.

The term “independently” means that where more than one substituent is selected from a number of possible substituents, those substituents may be the same or different.

The alternative definitions for the various groups and substitutent groups of Formulas (I) to (XVI), respectively, provided throughout the specification are intended to particularly describe each compound species disclosed herein, individually, as well as groups of one or more compound species. The scope of this invention includes any combination of these group and substituent group definitions.

### Enantiomers, Diastereomers and Polymorphs

The compounds according to Formulas (I) to (XVI) of the present invention may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula (I), or in any chemical structure illustrated herein, is not specified the structure is intended to encompass all individual stereoisomers and all mixtures thereof. Thus, compounds according to Formula (I) containing one or more chiral center may be used as racemic mixtures, enantiomerically enriched mixtures, or as enantiomerically pure individual stereoisomers.

Individual stereoisomers of a compound according to Formulas (I) to (XVI) which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation. When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

### Salts

Because of their potential use in medicine, the salts of the compounds of Formulas (I) through Formula (XVI) are preferably pharmaceutically acceptable salts. Suitable pharmaceutically acceptable salts include those described by Berge, Bighley and Monkhouse J. Pharm. Sci (1977) 66, pp 1-19.

When a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methyl benzoates, dinitrobenzoates, hydroxybenzoates, methoxybenzoates, phthalates, phenylacetates, phenylpropionates, phenylbutyrates, citrates, lactates, γ-hydroxybutyrates, glycollates, tartrates mandelates, and sulfonates, such as xylenesulfonates, methanesulfonates, propanesulfonates, naphthalene-1-sulfonates and naphthalene-2-sulfonates.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms.

Because the compounds of this invention may contain both acid and base moieties, pharmaceutically acceptable salts may be prepared by treating these compounds with an alkaline reagent or an acid reagent, respectively. Accordingly, this invention also provides for the conversion of one pharmaceutically acceptable salt of a compound of this invention, e.g., a hydrochloride salt, into another pharmaceutically acceptable salt of a compound of this invention, e.g., a sodium salt.

### Solvates

For solvates of the compounds of the invention, or salts thereof, that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

### Purity

Because the compounds of the present invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

### Synthetic Schemes and General Methods of Preparation

The present invention also relates to processes for making compounds of Formulas (I) to (XVI), respectively.

The present invention also relates to methods for the treatment of respiratory or respiratory tract diseases, which comprises administering to a subject in need thereof an effective amount of a compound of Formulas (I) to (XVI), respectively.

The compounds of the present invention may be obtained by using synthetic procedures illustrated in Schemes 1 to 6 below or by drawing on the knowledge of a skilled organic chemist.

The synthesis provided in these Schemes 1 to 6 are applicable for producing compounds of the invention as defined by Formulas (I) to (XVI), respectively, having a variety of different functional groups as defined employing appropriate precursors, which are suitably protected if needed, to achieve compatibility with the reactions outlined herein. Subsequent deprotection, where needed, affords compounds of the nature generally disclosed. While the Schemes 1 to 6, respectively, are shown with compounds only as defined therein, they are illustrative of processes that may be used to make the compounds of the invention.

Intermediates (compounds used in the preparation of the compounds of the invention) also may be present as salts. Thus, in reference to intermediates, the phrase “compound(s) of formula (number)” means a compound having that structural formula or a pharmaceutically acceptable salt thereof.

### Synthetic Schemes

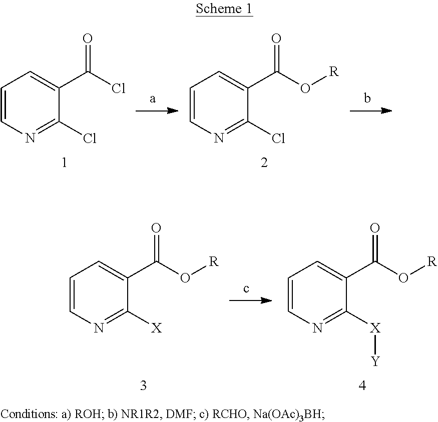

Scheme 1 represents a general scheme for the preparation of compounds according to Compounds (3) and (4) as shown above, where X is attached to the pyridine ring via a nitrogen atom. Compound 1, (2-chloronicotinoyl chloride—commercially available from Aldrich) depicted as starting material is available from commercial vendors. Reaction conditions are as described above in the scheme; however, the skilled artisan will appreciate that certain modifications in the reaction conditions and/or reagents used are possible.

Treatment of 2-chloronicotinoyl chloride 1 in an alcoholic solvent produces the desired ester 2. Ester 2 is further transformed to aminopyridine 3 via reaction with the appropriate amine. In the case where X contains a suitable protecting group, removal of the protecting group under the appropriate conditions and further transformation to other products of the present invention may be accomplished. Subsequent transformation of the amine function of the group X to the subsequent alkylamine XY can be performed with the appropriate aldehyde of Y via a reductive amination protocol. It will be appreciated by the skilled artisan that upon conversion to the alkylamine XY the resulting product may require further elaboration. This can include but is not limited to suitable protecting and functional group manipulations and reactions with alcohols, aryl halides, phenols, anilines, and amines.

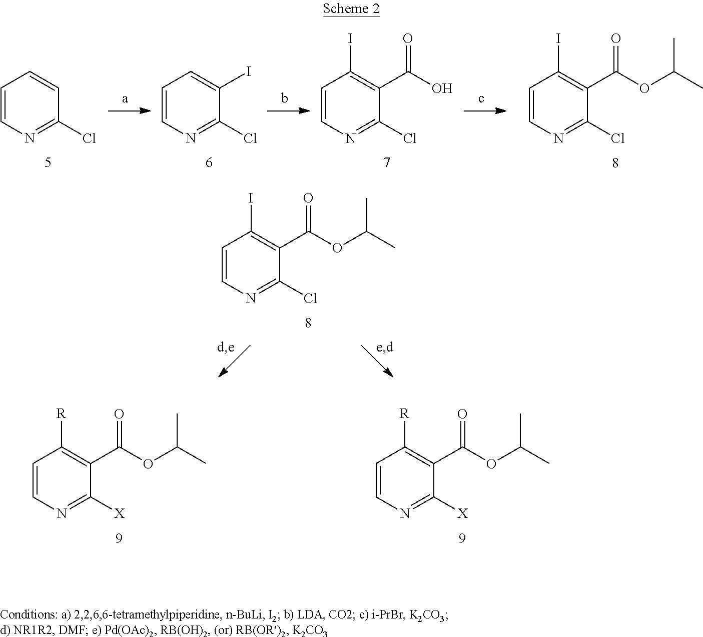

Scheme 2 represents a general scheme for the preparation of compounds according to—Compound (9) as defined above, where X is attached to the pyridine ring via a nitrogen atom and C4 is substituted. Compound 5, (2-chloropyridine) depicted as starting material is available from commercial vendors. Reaction conditions are as described above in the scheme; however, the skilled artisan will appreciate that certain modifications in the reaction conditions and/or reagents used are possible.

Deprotonation of 2-chloropyridine 5 followed by reaction with iodine produces the intermediate iodide 6. This is further transformed to the C3 acid/C4 iodide 7 via deprotonation using LDA followed by quenching with CO₂. The intermediate acid is then converted to the ester to produce the key compound 8 via treatment with isopropylbromide and potassium carbonate. With this material is hand, a 2 step sequence provides access to compounds of structure 9. In the first instance reaction with amine X where X may contain a suitable protecting group, followed by reaction of the C4 iodide provides access to 9 where the C4 substituent may be varied in the last step. Alternatively, the C4 substituent may be installed initially followed by incorporation of the C2 amine X allowing variation of the C2 position in the last step. Installation of the substituent R can be accomplished via a transition metal mediated coupling using an appropriate catalyst and coupling partner. As an example of such a transformation, for the case in Scheme 1 condition “e”, a Suzuki cross-coupling reaction can be completed using a boronic ester or acid in the presence of Pd(OAc)₂, Ph₃P, and K₂CO₃. Removal of any protecting group under the appropriate conditions and further transformation to other products may be accomplished. Subsequent transformation of the amine function of the group X to the subsequent alkylamine XY can be performed with the appropriate aldehyde of Y via a reductive amination protocol. It will be appreciated by the skilled artisan that upon conversion to the alkylamine XY the resulting product may require further elaboration. This can include but is not limited to suitable protecting and functional group manipulations and reactions with alcohols, aryl halides, phenols, anilines, and amines.

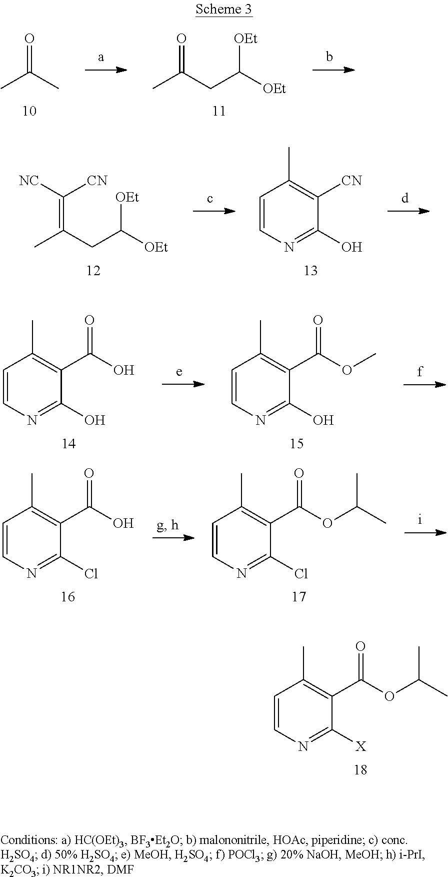

Scheme 3 represents a general scheme for the preparation of compounds according to Compound (18) as defined above, where X is attached to the pyridine ring via a nitrogen atom and C4 is substituted with a methyl group. Compound 10, (acetone) depicted as starting material is commercially available from commercial vendors. Reaction conditions are as described above in the scheme; however, the skilled artisan will appreciate that certain modifications in the reaction conditions and/or reagents used are possible.

Treatment of acetone with triethyl orthoformate produces the homologated ketone 11. Condensation with malononitrile and subsequent cyclization under acidic conditions produces pyridine 12. With this intermediate in hand, conversion through to the intermediate chloride 17 results from a series of functional group manipulations including hydrolysis of the nitrile to the acid, conversion of the acid to the methylester, reaction with POCI3 to produce the C2 chloride, hydrolysis of the ester to the acid and subsequent transformation of the acid to the isopropyl ester. Compound 17 can then be transformed to final products of the invention using conditions described in Scheme 3 above.

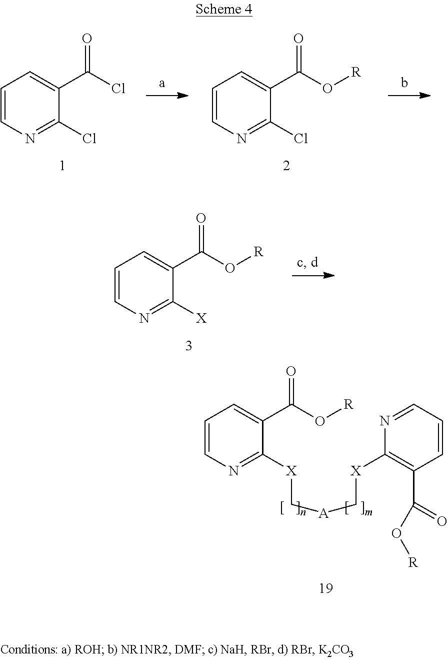

Scheme 4 represents a general scheme for the preparation of dimeric compounds (19) according to Compound 19 as defined above, where X is attached to the pyridine ring via a nitrogen atom. Compound 1, (2-chloronicotinoyl chloride) depicted as starting material is available from commercial vendors. Reaction conditions are as described above in the scheme; however, the skilled artisan will appreciate that certain modifications in the reaction conditions and/or reagents used are possible.

Treatment of 2-chloronicotinoyl chloride 1 in an alcoholic solvent produces the desired ester 2. Ester 2 is further transformed to aminopyridine 3 via reaction with the appropriate amine. In the case where X contains a suitable protecting group, removal of the protecting group under the appropriate conditions and further transformation to other products may be accomplished. In the case where the amine used to transform 2 to 3 is 3-Boc-aminopyrrolidine, installation of the alkyl group is achieved prior to removing the protecting group. With the protecting group removed, completion of the dimeric analogs 19 can be achieved via reaction with the appropriate benzyl or alkyl bromide under basic conditions. For the case where X is piperazine, the dimer analog can be made by reacting with the appropriate aldehyde bromide under basic conditions initially followed by reductive amination as described for Scheme 1.

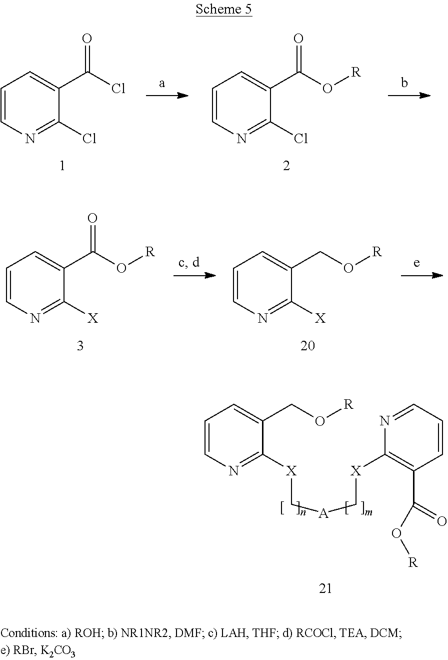

Scheme 5 represents a general scheme for the preparation of dimeric compounds (21) according to Compound 21, where X is attached to the pyridine ring via a nitrogen atom. Compound 1, (2-chloronicotinoyl chloride) depicted as starting material is available from commercial vendors. Reaction conditions are as described above in the scheme; however, the skilled artisan will appreciate that certain modifications in the reaction conditions and/or reagents used are possible.

Treatment of 2-chloronicotinoyl chloride 1 in an alcoholic solvent produces the desired ester 2. Ester 2 is further transformed to aminopyridine 3 via reaction with the appropriate amine. In the case where the amine used to transform 2 to 3 is 3-Boc-aminopyrrolidine, installation of the N-alkyl group can be achieved with the appropriate alkyl halide. Reduction of the ester to alcohol can then be achieved under reducing conditions using a reagent like lithium aluminium hydride. Formation of the ester is then accomplished via reaction with the appropriate acid chloride under basic conditions or with the appropriate acid in the presence of a coupling reagent. In the case where X contains a suitable protecting group, removal of the protecting group under the appropriate conditions and further transformation to other products may be accomplished. With the protecting group removed, completion of the dimeric analogs 21 can be achieved via reaction with the appropriate benzyl or alkyl bromide under basic conditions or in some cases via reaction with the appropriate dialdehyde under reductive amination conditions. For the case where X is piperazine, the dimer analog can be made by reacting initially with the appropriate aldehyde bromide under basic conditions followed by reductive amination as described for Scheme 1.

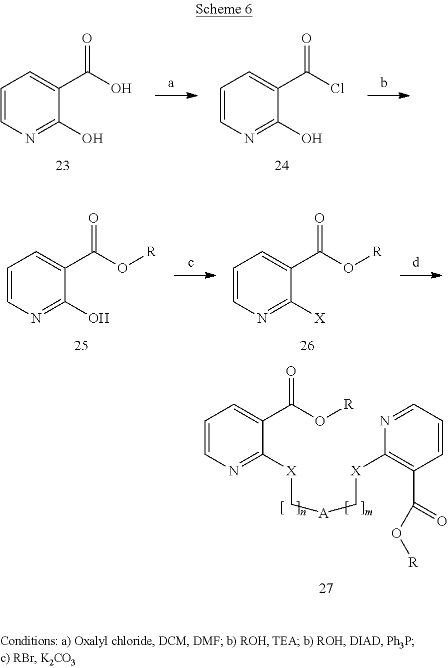

Scheme 6 represents a general scheme for the preparation of dimeric compounds (27) according to Compound (27) as defined above, where X is attached to the pyridine ring via an oxygen atom. Compound 23, (2-hydroxynicotinic acid) depicted as starting material is available from commercial vendors. Reaction conditions are as described above in the scheme; however, the skilled artisan will appreciate that certain modifications in the reaction conditions and/or reagents used are possible.

Treatment of 2-hydroxynicotinic acid 23 with oxalyl chloride produces the desired acid chloride 24. Acid chloride 24 is further transformed to ester 25 via reaction with the appropriate alcohol in presence of triethylamine. Conversion of the phenol to the requisite ether is then achieved under Mitsunobu conditions. In the case where X contains a suitable protecting group, removal of the protecting group under the appropriate conditions and further transformation to other products may be accomplished. With the protecting group removed, completion of the dimeric analogs 27 can be achieved via reaction with the appropriate benzyl or alkyl bromide under basic conditions or in some cases via reaction with the appropriate dialdehyde under reductive amination conditions. Alternatively, the dimer analog may be made by reacting initially with the appropriate aldehyde bromide under basic conditions followed by reductive amination as described for Scheme 1.

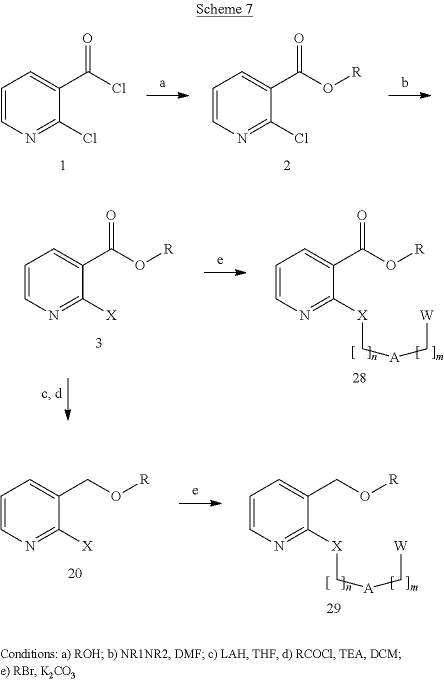

Scheme 7 represents a general scheme for the preparation of dimeric compounds (28) and (29), respectively. Compound 1, (2-chloronicotinoyl chloride) depicted as starting material is commercially available. Reaction conditions are as described above in the scheme; however, the skilled artisan will appreciate that certain modifications in the reaction conditions and/or reagents used are possible.

Treatment of 2-chloronicotinoyl chloride 1 in an alcoholic solvent produces the desired ester 2. Ester 2 is further transformed to aminopyridine 3 via reaction with the appropriate amine. In the case where the amine used to transform 2 to 3 is 3-Boc-aminopyrrolidine, installation of the N-alkyl group can be achieved with the appropriate alkyl halide.

In the case where X contains a suitable protecting group, removal of the protecting group under the appropriate conditions and further transformation to other products may be accomplished. With the protecting group removed, reaction with a benzyl or alkyl bromide, or benzyl or alkyl aldehyde, followed by an appropriate amine group “W” results completion of the dimeric analog (28).

Alternatively, reduction of the ester to alcohol can then be achieved under reducing conditions using a reagent like lithium aluminium hydride. Formation of the ester is then accomplished via reaction with the appropriate acid chloride under basic conditions or with the appropriate acid in the presence of a coupling reagent. In the case where X contains a suitable protecting group, removal of the protecting group under the appropriate conditions and further transformation to other products may be accomplished. With the protecting group removed, completion of the dimeric analog (29), respectively, can be achieved via reaction with the appropriate benzyl or alkyl bromide under basic conditions or in some cases via reaction with the appropriate dialdehyde under reductive amination conditions. For the case where X is piperazine, the dimer analog can be made by reacting initially with the appropriate aldehyde bromide under basic conditions followed by reductive amination as described for Scheme 1.

### Pharmaceutical Compositions, Dosage Forms and Regimens

The present invention relates to novel compounds of Formulas (I) to (XVI) and corresponding pharmaceutical compositions comprising compounds of Formulas (I) to (XVI), respectively.

The compounds of the invention will normally, but not necessarily, be formulated into a pharmaceutical composition prior to administration to a patient.

Accordingly, the present invention is directed to pharmaceutical compositions or formulations, which comprise a compound of the invention and pharmaceutically-acceptable excipient(s). In particular, the present invention also may relate to a pharmaceutical composition or formulation, which comprises a compound as defined by Formulas (I) to (XVI), respectively, or a pharmaceutically acceptable salt thereof, and pharmaceutically acceptable adjuvants, carriers or excipients, and optionally one or more other therapeutic ingredients.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula (I) or a salt, particularly a pharmaceutically acceptable salt, thereof). When prepared in unit dosage form, the pharmaceutical compositions or formulations may contain from 1 mg to 1000 mg of a compound of this invention.

The pharmaceutical compositions or formulations as defined herein typically contain one compound of the present invention. However, in certain embodiments, the pharmaceutical compositions may contain more than one compound of the present invention. In addition, the pharmaceutical compositions of the present invention may optionally further comprise one or more additional pharmaceutically active compounds.

As used herein, “pharmaceutically-acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically-acceptable are avoided. In addition, each excipient must of course be of sufficiently high purity to render it pharmaceutically-acceptable.

Suitable pharmaceutically-acceptable excipients will vary depending upon the particular dosage form chosen. In addition, suitable pharmaceutically-acceptable excipients may be chosen for a particular function that they may serve in the composition.

For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance. Moreover, pharmaceutical compositions, formulations, dosage forms, and the like, etc. may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The compounds of the invention and the pharmaceutically-acceptable excipient or excipients will typically be formulated into a dosage form adapted for administration to the patient by the desired route of administration.

With regard to the present invention, conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

The pharmaceutical compositions or formulations of the invention are prepared using techniques and methods known to those skilled in the art. Some of the methods commonly used in the art are described in Remington's Pharmaceutical Sciences (Mack Publishing Company).

In general, pharmaceutical compositions of the present invention are prepared using conventional materials and techniques, such as mixing, blending and the like.

The term “active agent” is defined for purposes of the present invention as any chemical substance or composition of the present invention, which can be delivered from the device into an environment of use to obtain a desired result.

The percentage of the compound in compositions can, of course, be varied as the amount of active in such therapeutically useful compositions is such that a suitable dosage will be obtained.

It will be appreciated that the actual preferred dosages of the compounds being used in the compositions of this invention will vary according to the particular composition formulated, the mode of administration, the particular site of administration and the host being treated.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet, etc.

In one aspect, compounds of Formulas (I) to (XVI) may also be administered by inhalation, that is by intranasal and oral inhalation administration. Appropriate dosage forms for such administration, such as an aerosol formulation or a metered dose inhaler, may be prepared by conventional techniques.

For administration by inhalation the compounds may be delivered in the form of an aerosol spray presentation from pressurized packs or a nebulizer, with the use of a suitable propellant, e.g. dichlorodifluoromethane, trichlorofluoromethane, dichlorotetrafluoroethane, a hydrofluroalkane such as tetrafluoroethane or heptafluoropropane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of a compound of the invention and a suitable powder base such as lactose or starch.

Dry powder compositions for topical delivery to the lung by inhalation may, for example, be presented in capsules and cartridges of for example gelatine or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di or poly-saccharides (e.g. lactose or starch). Use of lactose is preferred. Each capsule or cartridge may generally contain between 20 μg-10 mg of the compound of formula (I) optionally in combination with another therapeutically active ingredient. Alternatively, the compound of the invention may be presented without excipients.

Suitably, the packing/medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

In the case of multi-dose delivery, the formulation can be pre-metered (e.g. as in Diskus, see GB 2242134, U.S. Pat. Nos. 6,632,666, 5,860,419, 5,873,360 and 5,590,645 or Diskhaler, see GB 2178965, 2129691 and 2169265, U.S. Pat. Nos. 4,778,054, 4,811,731, 5,035,237, the disclosures of which are hereby incorporated by reference) or metered in use (e.g. as in Turbuhaler, see EP 69715 or in the devices described in U.S. Pat. No. 6,321,747 the disclosures of which are hereby incorporated by reference). An example of a unit-dose device is Rotahaler (see GB 2064336 and U.S. Pat. No. 4,353,656, the disclosures of which are hereby incorporated by reference).

The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) or (Ia) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament there from.

In one aspect, the multi-dose blister pack comprises plural blisters arranged in generally circular fashion on a disc-form blister pack. In another aspect, the multi-dose blister pack is elongate in form, for example comprising a strip or a tape. In one aspect, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined there between and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognised as being extremely effective in removing particles from the nose in a rapid manner, for example, within 10-30 minutes from the time the particles enter the nose. Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regime for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

In one aspect, the means for applying a formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the pre-compression pump is that atomization of the spray is ensured as it will not release the formulation until the threshold pressure for effectively atomizing the spray has been achieved. Typically, the VP7 model may be used with a bottle capable of holding 10-50 ml of a formulation. Each spray will typically deliver 50-1000 of such a formulation, therefore, the VP7 model is capable of providing at least 100 metered doses.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of Formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants, e.g., oleic acid or lecithin and cosolvents, e.g. ethanol. Pressurised formulations will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means e.g., by micronization. The desired fraction may be separated out by air classification or sieving. Suitably, the particles will be crystalline in form. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulization may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

For all methods of use disclosed herein for the compounds of Formulas (I) to (XVI), the daily oral dosage regimen will preferably be from about 0.05 to about 80 mg/kg of total body weight, preferably from about 0.1 to 30 mg/kg, more preferably from about 0.5 mg to 15 mg/kg, administered in one or more daily doses. For example, the daily parenteral dosage regimen about 0.1 to about 80 mg/kg of total body weight, preferably from about 0.2 to about 30 mg/kg, and more preferably from about 0.5 mg to 15 mg/kg, administered in one or more daily doses. The daily topical dosage regimen will preferably be from 0.01 mg to 150 mg, administered one to four times daily. The daily inhalation dosage regimen will preferably be from about 0.05 microgram/kg to about 5 mg/kg per day, or from about 0.2 microgram/kg to about 20 microgram/kg, administered in one or more daily doses.

It will also be recognized by one of skill in the art that the optimal quantity and spacing of individual dosages of a compound of Formulas (I) to (XVI), respectively, or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound of Formulas (I) to (XVI), respectively, or a pharmaceutically acceptable salt thereof given per day for a defined number of days, can be ascertained by those skilled in the art using conventional course of treatment determination tests.

The amount of a compound of Formulas (I) to (XVI), respectively, or a pharmaceutically acceptable salt thereof which is required to achieve a therapeutic effect will, of course, vary with the particular compound, the route of administration, the subject under treatment, and the particular disorder or disease being treated.

The compounds of the present invention may be administered by inhalation at a dose of from 0.0005 mg to 400 mg. In another aspect, compounds of the present invention may be administered by inhalation at a dose of from 0.00 5 mg to 40 mg, such as at a dose of from 0.05 mg to 0.5 mg. The dose range for adult humans is generally from 0.0005 mg to 10 mg per day; such as at a dose of from 0.01 mg to 1 mg per day or from 0.05 mg to 0.5 mg per day.

### Administration

Treatment regimen for the administration of compounds, pharmaceutical compositions, or controlled-release formulations or dosage forms of the present invention also may be determined readily by those with ordinary skill in art.

The quantity of the compound, pharmaceutical composition, or dosage form of the present invention administered may vary over a wide range to provide in a unit dosage in an effective amount based upon the body weight of the patient per day to achieve the desired effect and as based upon the mode of administration.

The scope of the present invention includes all compounds, pharmaceutical compositions, or controlled-release formulations or dosage forms, which is contained in an amount effective to achieve its intended purpose. While individual needs vary, determination of optimal ranges of effective amounts of each component is within the skill of the art.

The compounds of the invention may be administered by any suitable route of administration, including both systemic administration and topical administration. Systemic administration includes oral administration, parenteral administration, transdermal administration, rectal administration, and administration by inhalation.

Parenteral administration refers to routes of administration other than enteral, transdermal, or by inhalation, and is typically by injection or infusion. Parenteral administration includes intravenous, intramuscular, and subcutaneous injection or infusion.

Inhalation refers to administration into the patient's lungs whether inhaled through the mouth or through the nasal passages.

In one aspect, pharmaceutical compositions, formulations, dosages, dosage forms or dosing regimens of the present invention are adapted for administration by inhalation.

Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect.

Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

### Methods of Treatment

The present invention also relates to uses or methods for the treatment of respiratory or respiratory tract diseases, which comprises administering to a subject in need thereof an effective amount of a compound of Formulas (I) to (XVI), respectively.

As used herein, “patient” refers to a human or other mammal.

In one aspect, the present invention is directed to a use or a method for treatment of respiratory or respiratory tract diseases selected from asthma, allergen-induced asthmatic reactions, cystic fibrosis, bronchitis, chronic bronchitis, chronic obstructive pulmonary disease (COPD), cough, adult respiratory distress syndrome (ARDS), chronic pulmonary inflammation, rhinitis and upper respiratory tract inflammatory disorders (URID), ventilator induced lung injury, silicosis, pulmonary sarcoidosis, idiopathic pulmonary fibrosis or bronchopulmonary dysplasia. Specific types of coughs which may be treated by compounds of the present invention, include, but are not limited to dry cough, wet cough, croupy cough, or chest cough.

In one aspect, the present invention relates to a use or a method for treating chronic obstructive pulmonary diseases (COPD), which comprises administering an effective amount of a compound of Formulas (I) to (XVI), or pharmaceutical composition of the present invention, respectively, to a subject in need thereof.

In another aspect, the present invention relates to a use or a method for treating cough, which comprises administering to a subject in need thereof an effective amount of a compound of Formulas (I) to (XVI), respectively.

The compounds, pharmaceutical compositions, controlled release formulations or dosage forms prepared according to the present invention can be used to treat warm-blooded animals, such as mammals, which include humans.

In accordance with any of the methods or uses of administration of the present invention, the term a “therapeutically effective amount”, as used herein, generally includes within its meaning a non-toxic but sufficient amount of the particular drug to which it is referring to provide the desired therapeutic effect. The exact amount required will vary from subject to subject depending on factors such as the patient's general health, the patient's age, etc.

Active drug or therapeutic agents or compounds, such as those described above may be prepared according to processes or methods taught by either the present disclosure or processes or methods known to those of skill in the art.

### Combination Therapies

Active drug or therapeutic agents, when employed in combination with the compounds, or pharmaceutical compositions of the present invention, may be used or administered, for example, in dosage amounts indicated in the Physicians' Desk Reference (PDR) or as otherwise determined by one of ordinary skill in the art.

In the context of this specification, the term “simultaneously” when referring to simultaneous administration of the relevant drugs means at exactly the same time, as would be the case, for example in embodiments where the drugs are combined in a single preparation. In other embodiments, “simultaneously” can mean one drug taken a short duration after another, wherein “a short duration” means a duration which allows the drugs to have their intended synergistic effect.

In light of the foregoing, the present invention also relates to a combination therapy, which may be a comprised of a simultaneous or co-administration, or serial administration of a combination of compounds or pharmaceutical compositions of the present invention with other active drug or therapeutic agents, such as described above, and where such administration also is determined by one of ordinary skill in the art.

In addition, the present invention also relates to a combination therapy for the treatment or prevention of reparatory tract or respiratory diseases as described herein, which is comprised of a composition, dosage form or formulation formed from a synergistic combination or mixture of compounds, controlled release compositions, dosage forms or formulations of the present invention and another active drug or therapeutic agent or agents as those described above and optionally which comprises pharmaceutically acceptable carrier, diluent or adjuvant. In such an aforementioned combination composition, dosage form or formulation of the present invention, each of the active drug components are contained in therapeutically effective and synergistic dosage amounts.

The Examples set forth below are illustrative of the present invention and are not intended to limit, in any way, the scope of the present invention.

### EXAMPLES

The following examples illustrate the invention. These examples are not intended to limit the scope of the present invention, but rather to provide guidance to the skilled artisan to prepare and use the compounds, compositions, and methods of the present invention.

While particular embodiments of the present invention are described, the skilled artisan will appreciate that various changes and modifications can be made without departing from the spirit and scope of the invention. Some of chemical compounds or pharmaceutically acceptable salts thereof of the present invention may be made by different chemical reaction methods or preparative procedures. Some examples of compounds prepared by different experimental procedures are found in, but not limited to representative Examples 225 and 474, 368 and 469, 365 and 468, 407 and 471 and the like.

### Biology and Biological Assays

### Mechanism of Action of the Present Invention:

The cough reflex protects the airway from potential harm by aiding the clearance of luminal debris. Within the airway epithelium, irritant sensing vagal nerve endings transmit information arising from the presence of tussive stimuli to the brain stem evoking an urge to cough. Coughing is produced in a variety of airway diseases, which may enhance and intensify the cough response. Chronic cough, often thought as dry and unproductive, is associated with progressive irreversible lung damage such as occurs in chronic obstructive pulmonary disease (COPD). The persistence and intensity of this form of cough robs patients of quality of life.

Propagation of nerve impulses arising from tussive stimuli is mediated, at least in part, via voltage-gated Na⁺ channels (NaV). Generation of the action potential is blocked by local anesthetics such as Lidocaine. Lidocaine reduces the inward sodium current which elicits neuronal impulses (Butterworth et al., 1990; Catterall, 1987; Hille, 1966; Taylor, 1959). Indeed, blockade of neuronal Na⁺ channels is one of the most powerful and well described analgesic principles (Catterall et al., 2005). Lidocaine, a pan-NaV inhibitor, is used to minimize gagging and cough during bronchoconscopy (Reed, 1992) and to limit airway intubation-induced post operative cough and sore throat (Diachun et al., 2001). There is evidence suggesting that short-term administration of intravenous lidocaine may produce pain relief that far exceeds both the duration of infusion and the half-life of the drug (McCleane, 2007). Although widely investigated, the mechanism remains unknown. One possibility is that local anesthetics inhibit central sensitization, i.e., the long-term increase in the excitability of the central nervous system in response to on-going or repeated activation of nociceptors. Blockade of sensory nerve input even for a short time would allow restoration of normal nerve function, a similar long-lasting effect on intractable dry cough could be expected.

### Biological Assays

The ability of the compounds of the invention to modulate the voltage-gated sodium channel subtype NaV 1.3 and NaV 1.7 may be determined by the following assay.

### Cell Biology

Stable cell lines expressing hNaV1.3 channels were created by transfecting CHO cells with the pCIN5-hNav1.3 vector using the lipofectamine (Invitrogen) transfection method. pCIN5 is a bicistronic vector for the creation of mammalian cell lines that predisposes all neomycin resistant cells to express recombinant protein (see Rees S., Coote J., Stable J., Goodson S., Harris S. & Lee M. G. (1996) Biotechniques, 20, 102-112) by virtue of the recombinant cDNA being linked to the neomycin-selectable marker cDNA downstream of the CMV promoter (for full details see Chen Y H, Dale T J, Romanos M A, Whitaker W R, Xie X M, Clare J J. Cloning, distribution and functional analysis of the type III sodium channel from human brain Eur J Neurosci, 2000 December; 12, 4281-9). Cells were cultured in Iscove's Modified Dulbecco's Medium (Invitrogen, 21980-032) adding, 10% Dialized Fetal Bovine Serum (PAA, A15-107), 1% L-glutamine (Invitrogen, 25030-024), 1% Penicillin-Streptomycin (Invitrogen, 15140-122), 1% non-essential amino acids (Invitrogen, 11140-035), 2% HT supplement (Invitrogen, 41065-012) and 400 ug/ml of G418 (PAA, P11-012). Cells were grown and maintained at 37° C. in a humidified environment containing 5% CO2 in air. Cells were detached from the T175 culture flask for passage and harvesting using Versene (Invitrogen, 15040-033).

### Cell Preparation

Cells were grown to 60-95% confluence in a T175 flask. Cells were removed from the incubator and the media was aspirated. Cells were washed with 3 ml of warmed (37° C.) Versene and then 1.5 ml of warmed (37° C.) Versene was added to the flask for 6 min. The flask was tapped to dislodge cells and 10 ml of warmed (37° C.) DPBS (Invitrogen, 14040) was added to prepare a cell suspension. Cell suspension was then placed into a 15 ml centrifuge tube and centrifuged for 2 min at 1000 rpm. After centrifugation, the supernatant was removed and the cell pellet was resuspended in 5 ml of warmed (37° C.) DPBS using a 5 ml pipette to break up the pellet.

### Electrophysiology

Currents were recorded at room temperature using the IonWorks Quattro™ planar array electrophysiology technology (Molecular Devices Corp.) with PatchPlate™ PPC for Ionworks Quattro (Molecular Devices, 9000-0902). Stimulation protocols and data acquisition were carried out using a microcomputer (Dell Pentium 4). In order to determine planar electrode hole resistances (Rp), a 10 mV voltage step was applied across each well. These measurements were performed before cell addition. After cell addition a seal test was performed by applying a voltage step from −100 mV to −90 mV for 80 ms prior to antibiotic amphotericin-B solution (Sigma, P11-012) circulation to achieve intracellular access. Leak subtraction was conducted in all experiments by applying a 80 ms hyperpolarizing (10 mV) prepulse followed by a 80 ms at the holding potential before the test pulses, to measure leak current. Test pulses stepping from the holding potential of −90 mV to 0 mV were applied for 20 ms and repeated 10 times at a frequency of 10 Hz. In all experiments, the test pulse protocol was performed in the absence (pre-read) and presence (post-read) of a compound. Pre- and post-reads were separated by a compound addition followed by a 3 minute incubation.

### Solutions and Drugs

The intracellular solution contained the following (in mM): K-gluconate 100, KCl, 40, MgCl2 3.2, EGTA 3, HEPES 5, adjusted to pH 7.5. Amphotericin-B solution was prepared as 50 mg/ml stock solution in DMSO and diluted to a final working concentration of 0.1 mg/ml in intracellular solution. The external solution was Dulbecco's PBS (Invitrogen, 14040) and contained the following (in mM): CaCl2 0.90, KCl 2.67, KH2PO4 1.47, MgCL6H2O 0.493, NaCl 136.9, Na3PO4 8.06, with a pH of 7.4. Compounds were prepared in DMSO as 10 mM stock solutions and subsequent 1:3 serial dilutions was performed. Finally the compounds were diluted 1:100 in external solution containing 0.05% pluronic acid.

### Data Analysis

The recordings were analysed and filtered using both seal resistance (>40 MΩ) and peak current amplitude (>200 pA) in the absence of compound to eliminate unsuitable cells from further analysis. Paired comparisons between pre-drug and post-drug additions were used to determine the inhibitory effect of each compound. Data were normalized to the high control (1% DMSO) and low control (0.3 uM Tetrodotoxin from Tocris, 1069). The normalized data were analysed by using ActivityBase software. The concentrations of compounds required to inhibit current elicited by the 1^{st} depolarizing pulse by 50% (tonic pIC50) were determined by fitting of the four parameter logistic function to the concentration response data. In addition the use-dependent inhibitory properties of the compounds were determined by assessing the effect of compounds on the 10^{th} versus 1^{st} depolarizing pulse. The ratio of the 10^{th} over 1^{st} pulse was calculated in the absence and presence of drug and the % use-dependent inhibition calculated. The data was fitted using the same equation as for the tonic pIC₅₀ and the concentration producing 15% inhibition (use-dependent pUD₁₅) calculated.

The following compounds identified by Example numbers were tested and found to have pUD₁₅ of 5.5 or greater against NaV1.3:
3-8, 10-11, 17, 19-20, 22-24, 27, 30, 38, 48, 51-52, 54-55, 58-61, 64, 67-68, 70, 72-74, 80, 86, 88, 90-91, 93-96, 98, 111-112, 114-119, 122-123, 125-128, 136, 139, 144, 148, 152, 169, 172-173, 175-176, 179-181, 183, 187, 188, 195, 197, 199, 203-204, 212, 220-223, 226, 228-229, 231-238, 244-245, 248, 250-251, 257, 258, 260-262, 264-266, 270-282, 285, 287, 289-291, 295-296, 298-299, 301, 303-307, 310-313, 316, 319, 322-328, 330-335, 347, 352, 357, 364-366, 368, 371, 373-377, 379-386, 389-395, 399-401, 403-404, 407, 409-412, 414, 417, 423, 428, 433, 436, 438, 442, 447, 449, 453, 455, 460, 463, 464, 466, 467, 468, 470, 471, 475, 476, 477, 478, 479, 482, 483, 485, 486, 488, 489, 490, 491, 492, 493, 494, 497, 498, 499, 500, 501, 502, 503, 504, 505, 508, 511, 513, 514, 515, 516, 517, 518, 520, 522, 523, 524, 527, 528, 542.

The following compounds were tested and found to have pUD₁₅ of 5.5 or greater against NaV1.7:
4-8, 10-11, 14, 19-20, 23-24, 30, 38, 48, 51, 52, 54-55, 60-61, 64, 67-68, 70, 72-74, 81, 85-86, 88, 90-91, 93-95, 111, 115-118, 122-123, 125-128, 144, 152, 169, 173, 176-177, 181, 183, 190-191, 199, 204, 212, 216, 220-221, 226, 231-232, 234, 236-237, 244, 251, 256-257, 260-262, 265-266, 270-271, 274, 276-280, 282, 285, 287, 289, 291, 295, 298, 299, 303-306, 310-311, 313, 319, 322-325, 330, 332-333, 335, 357, 364, 365, 368, 373-375, 377, 379-384, 386-387, 389, 391-392, 394-395, 399, 409-410, 412, 414, 417, 419, 423, 425, 436-437, 442, 447, 449, 453, 460, 464, 467, 468, 470, 471, 472, 475, 476, 477, 479, 482, 488, 489, 490, 491, 492, 493, 497, 500, 501, 502, 508, 513, 514, 515, 516, 517, 518, 519, 520, 521, 523, 530, 532, 537, 542

The following compounds were tested and found to have pUD15 of 4-4.99 against NaV1.3:
12, 31, 34, 36-37, 43, 45-47, 49-50, 56, 62, 65-66, 69, 76-77, 83, 99-104, 106-110, 124, 129, 133, 143, 145-147, 150, 154-155, 158, 160, 162, 164, 166, 168, 170, 185-186, 189, 194, 196, 200, 208, 210, 213, 215, 218, 2390-242, 246, 252, 254, 263, 268, 293, 300, 314-315, 318, 321, 329, 337, 339, 341, 344-345, 348, 355, 358, 361, 363, 370, 378, 406, 408, 416, 418, 420, 422, 427, 431-432, 437, 439, 441, 444-446, 450-451, 454, 456-457, 462, 473, 484, 510, 533-534, 538, 539, 540.

The following compounds were tested and found to have pUD15 of 5-5.99 against NaV1.3:
1-4, 8-10, 13-18, 21-22, 24-29, 32-33, 39-40, 42, 48, 53, 58-59, 61, 63-64, 71, 75, 78-82, 85, 87, 89, 93-94, 96-98, 105, 111-112, 114, 116, 119-123, 126, 130, 135-136, 138-139, 141, 144, 148-149, 151-152, 156-157, 169, 171-184, 187-188, 190-191, 193, 195, 197, 199, 202-203, 205-207, 211-212, 214, 216, 219-230, 232-236, 238, 243-245, 247-248, 250-251, 253, 255-262, 264, 266-267, 269-279, 281-282, 284-288, 290-292, 294, 296-297, 301-303, 306-309, 312-313, 316-317, 319-320, 322-323, 325-328, 331, 333-334, 336, 340, 342, 346-347, 349-354, 356-357, 359-360, 362, 364-366, 368, 371-372, 374-377, 383, 385-405, 407, 409-414, 417, 419, 423, 425-426, 428-430, 433-436, 438, 440, 443, 447, 449, 453, 455, 459-461, 463, 464, 465, 466, 467, 468, 472, 474, 475, 479, 480, 481, 483, 485, 486, 487, 488, 489, 490, 492, 493, 494, 495, 496, 498, 499, 500, 502, 504, 506, 507, 509, 511, 512, 514, 516, 517, 518, 519, 521, 524, 525, 526, 528, 529-531, 537, 541.

The following compounds were tested and found to have pUD15 of 6-7.5 against NaV1.3:
5-7, 11, 19-20, 23, 30, 38, 51-52, 54-55, 60, 67-68, 70, 72-74, 86, 88, 90-91, 95, 115, 117-118, 125, 127-128, 204, 231, 237, 265, 280, 289, 295, 298-299, 304-305, 310-311, 324, 330, 332, 335, 373, 379-382, 384, 442, 447, 468, 470, 471, 476, 477, 478, 482, 491, 497, 501, 503, 505, 508, 513, 515, 520, 522, 523, 527, 542.

The following compounds were tested and found to be inactive with respect to use-dependent potency against NaV1.3:
35, 44, 84, 92, 113, 131-132, 134, 137, 140, 142, 153, 159, 161, 163, 165, 167, 192, 198, 201, 209, 217, 249, 283, 338, 343, 367, 369, 415, 421, 424, 448, 452, 458, 532, 535.

Examples 41 and 57 were not tested for potency against NaV1.3.

The following compounds were tested and found to have pUD15 of 4-4.99 against NaV1.7:
13, 21, 25-26, 29, 31, 35-36, 39, 47, 50, 53, 56, 65, 71, 75, 78-79, 87, 89, 97, 99-102, 107-110, 121, 124, 130, 133, 135-138, 142-143, 145, 147, 157, 168, 170, 172, 185, 198, 207-208, 210, 218-219, 240-242, 246, 252, 286, 308, 312, 314, 317-318, 327, 329, 331, 342-343, 346, 352, 354, 359, 362, 388, 398, 402, 406, 408, 427, 432-433, 440, 443, 456-457, 459, 480, 484, 495, 498, 503, 506, 512, 526, 529, 539, 540, 533-534.

The following compounds were tested and found to have pUD15 of 5-5.99 against NaV1.7:
1-4, 8-10, 12, 14-18, 20, 22, 24, 27-28, 30, 32-34, 38, 40, 42, 46, 48, 58-59, 61-64, 72-74, 80-81, 85, 90, 93-94, 96, 98, 103, 105, 111-112, 114-116, 118-120, 122-123, 126-127, 129, 139, 141, 144, 148-149, 151-152, 169, 171, 173-184, 188, 190, 195, 197, 199, 203, 206, 211-212, 214, 216, 220-239, 243-245, 247-248, 251, 253-262, 266, 270-272, 274-278, 282, 284-285, 287-288, 290-292, 298-299, 302-307, 309, 313, 316, 319-320, 322-323, 325-326, 328, 333-334, 340, 347, 350, 357, 360, 364-366, 368, 371, 373-378, 382-383, 385-387, 389-395, 397, 399-401, 403-405, 407, 409-410, 412, 414, 417, 419, 423, 425-426, 428-430, 434-439, 441, 446-447, 449, 451, 453, 455, 460, 463, 464, 465, 466, 467, 468, 472, 474, 475, 479, 481, 489, 490, 493, 494, 496, 497, 500, 504, 505, 507, 509, 511, 513, 514, 515, 516, 517, 518, 520, 521, 522, 523, 524, 525, 530, 532, 538, 537, 541, 542.

The following compounds were tested and found to have pUD15 of 6-7.5 against NaV1.7:
5-7, 11, 19, 23, 51-52, 54-55, 60, 67-68, 70, 86, 88, 91, 95, 117, 125, 128, 191, 204, 265, 279-280, 289, 295, 310-311, 324, 330, 332, 335, 379-381, 384, 442, 468, 470, 471, 476, 477, 482, 488, 491, 492, 501, 502, 508, 519.

The following compounds were tested and found to be inactive with respect to use-dependent potency against NaV1.7:
37, 44, 49, 66, 69, 76-77, 82-84, 92, 104, 106, 113, 131, 134, 140, 146, 150, 153-156, 158, 160, 162-167, 186-187, 189, 192-194, 196, 200-202, 205, 209, 213, 215, 217, 249-250, 283, 293, 296-297, 300-301, 315, 321, 336-339, 341, 344-345, 348-349, 351, 353, 355-356, 358, 361, 363, 367, 369-370, 372, 396, 411, 413, 416, 418, 420, 424, 431, 444-445, 448, 450, 452, 454, 458, 462, 473, 478, 483, 485, 486, 487, 499, 510, 527, 528, 535.

The following examples were not tested for potency against NaV1.7:
41, 43, 45, 57, 132, 159, 161, 263-264, 267-269, 273, 281, 294, 415, 421-422, 461 and 531.

### Guinea Pig Cough Method

Male Hartley Guinea pigs (n=6-8/group), weight range 600-700 gm were used in this study. After balancing transducers and air flow into the whole body plethysmograph chambers, the animals (after the appropriate pretreatment time) were placed into each of 4 chambers and allowed to acclimate to their new environment for approximately 5 minutes. Citric Acid (0.2M) was aerosolized into each chamber for 5 minutes and the animals remained in the chambers an additional 8 minutes. The number of coughs are counted by the computer software during the entire 13 minute time period. The software records each cough incident and records the time of the incident and totals the number of coughs for each animal during the test period (13 minutes). Results are summarized into a spread sheet.

### Intratracheal Dosing in Guinea Pigs.

Dosing—Animals are anesthetized (with 5% isoflurane using 95%02) and placed in the supine position. The drug/vehicle is then administered through the trachea. The trachea is intubated with a steel gavage needle (1.5 inch, 22 gauge, small ball) and 2000 of dosing solution or suspension is delivered. For intratracheal microspray applications (solutions only), the Penn-Century MicroSprayer® (19 gauge stainless steel tubing, see picture below) device is used to deliver 2004 The animals are visually monitored during the recovery process, which typically occurs within two minutes.

### Compound Examples

### General

Unless otherwise noted, all starting materials were obtained from commercial suppliers and used without further purification. Unless otherwise indicated, all temperatures are expressed in ° C. (degrees Centigrade). Unless otherwise indicated, all reactions are conducted under an inert atmosphere at ambient temperature.

All temperatures are given in degrees Celsius, all solvents are highest available purity and all reactions run under anhydrous conditions in an argon (Ar) or nitrogen (N₂) atmosphere where necessary.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. The CombiFlash system used for purification in this application was purchased from Isco, Inc. CombiFlash purification was carried out using prepacked silica gel columns, a detector with UV wavelength at 254 nm and a variety of solvents or solvent combinations.

Preparative HPLC was performed using a Gilson Preparative System with variable wavelength UV detection or an Agilent Mass Directed AutoPrep (MDAP) system with both mass and variable wavelength UV detection. A variety of reverse phase columns, e.g., Shimadzu 15 u m 250*21.2 mm, Luna 5u C18(2) 100A, SunFire™ C18, XBridge™ C18 were used in the purification with the choice of column support dependent upon the conditions used in the purification. The compounds are eluted using a gradient of acetonitrile and water. Neutral conditions used an acetonitrile and water gradient with no additional modifier, acidic conditions used an acid modifier, usually 0.05% or 0.1% TFA (added to both the acetonitrile and water) and basic conditions used a basic modifier, usually 10 mmol/L NH₄HCO₃, 0.04% NH₃H₂O or 0.1% NH₄OH (added to the water).

Analytical HPLC was run using an Agilent system with variable wavelength UV detection using reverse phase chromatography with an acetonitrile and water gradient with a 0.05 or 0.1% TFA modifier (added to each solvent). LC-MS was determined using Aglient 6110 quadrupole LC/MS, a PE Sciex Single Quadrupole LC/MS API-150 or a Waters. The compound is analyzed using a reverse phase column, e.g., Xbridge-C18, Sunfire-C18, Thermo Aquasil/Aquasil C18, Acquity HPLC C18, Thermo Hypersil Gold eluted using an acetonitrile and water gradient with a low percentage of an acid modifier such as 0.02% TFA or 0.1% formic acid.

Nuclear magnetic resonance spectra were recorded at 400 MHz using a Bruker AVANCE3 400, Bruker AC 400 or Brucker DPX400 spectrometer. CDCl₃ is deuteriochloroform, DMSO-D₆ is hexadeuteriodimethylsulfoxide, and CD₃OD is tetradeuteriomethanol. Chemical shifts are reported in parts per million (δ) downfield from the internal standard tetramethylsilane (TMS) or calibrated to the residual proton signal in the NMR solvent (e.g., CHCl₃ in CDCl₃). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

Heating of reaction mixtures with microwave irradiations was carried out on a Smith Creator (purchased from Personal Chemistry, Forboro, Mass., now owned by Biotage), an Emrys Optimizer (purchased from Personal Chemistry) or an Explorer (purchased from CEM, Matthews, N.C.) microwave.

Cartridges or columns containing polymer based functional groups (acid, base, metal chelators, etc) can be used as part of compound workup. The “amine” columns or cartridges are used to neutralize or basify acidic reaction mixtures or products. These include NH2 Aminopropyl SPE-ed SPE Cartridges available from Applied Separations and diethylamino SPE cartridges available from United Chemical Technologies, Inc.

In some cases, purifications and analyses of materials were carried out using the following instruments:

### LC-MS Analysis

The LC/MS of Intermediates and Examples were performed using the following equipment and conditions:

### Liquid Chromatograph:

System: Shimadzu LC system with SCL-10A Controller and dual UV detector
Autosampler: Leap CTC with a Valco six port injector

### Column: Aquasil/Aquasil (C18 40×1 mm)

Inj. Volume: 2.0 μL

### Solvent A: H₂O, 0.02% TFA

### Solvent B: MeCN, 0.018% TFA

Gradient: linear

### Channel A: UV 214 nm

### Channel B: ELS

| Step | Time (min) | Dura. (min) | Flow (μL/min) | Sol. A | Sol. B |
|---|---|---|---|---|---|
| 0 | 0.00 | 0.00 | 300.00 | 95.00 | 5.00 |
| 1 | 0.00 | 0.01 | 300.00 | 95.00 | 5.00 |
| 2 | 0.01 | 3.20 | 300.00 | 10.00 | 90.00 |
| 3 | 3.21 | 1.00 | 300.00 | 10.00 | 90.00 |
| 4 | 4.21 | 0.10 | 300.00 | 95.00 | 5.00 |
| 5 | 4.31 | 0.40 | 300.00 | 95.00 | 5.00 |

Mass Spectrometer:

### Instrument: PE Sciex Single Quadrupole LC/MS API-150

### Polarity: Positive

Acquisition mode: Profile

### Preparatory HPLC

Automated preparatory HPLC purifications were conducted using a Gilson® semi-preparative HPLC system under the following conditions:
Column: 75×33 mm I. D., S-5 um, 12 nm
Flow rate: 30 mL/min
Injection Volume: 0.800 mL
Room temperature
The eluent was a mixture composed of solvents A and B. Either one of three different solvent combinations were used:
   TFA conditions
      Solvent A: 0.1% trifluoroacetic acid in water
      Solvent B: 0.1% trifluoroacetic acid in acetonitrile
   NH₄OH conditions
   Solvent A: 0.1% NH₄OH in water
   Solvent B: 0.1% NH₄OH in acetonitrile
Neutral conditions
   Solvent A: 0.1% NH₄OH in water
   Solvent B: 0.1% NH₄OH in acetonitrile

### Automated Flash Chromatography

The automated flash chromatography purifications were conducted with a CombiFlash® Companion® personal flash chromatography system under the following conditions:
Silica cartridge:
   Size, 4, 12, 40, 80 or 120 g depending on the amount of material to be purified
Flow rate: Between 4 and 85 mL/min
Room temperature
The eluent was a mixture composed of solvents A and B:
   Solvent A: Hexane
   Solvent B: Ethyl acetate

### Mass-Directed Auto Prep HPLC

The Mass-Directed Auto Prep HPLC (MDAP) purifications were conducted with an Agilent preparatory HPLC-MS system under the following conditions:
Column: ZORBAX Eclipse XDB-C18 (21.2×50 mm)
Flow rate: 20 mL/min
Injection volume: 900 uL
Temperature: 30° C.
absorption wavelength: 230 nm
The eluent was a mixture composed of solvents A and B:
   Solvent A: 0.1% trifluoroacetic acid in water
   Solvent B: 0.1% trifluoroacetic acid in acetonitrile

### Monomers and Corresponding Intermediates

### Intermediates

### Intermediate 1: 4,4-bis(ethyloxy)-2-butanone

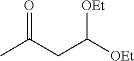

BF₃Et₂O (53.8 g, 378.8 mmol) was added dropwise over 15 min to a cooled (−40° C.) solution of HC(OEt)₃ (51.0 g, 344.4 mmol) in CH₂Cl₂ (200 mL). Stirring was continued for 10 min at −40° C. then the solution was transferred to an ice-water bath and stirred at 0° C. for 20 min. The mixture was cooled to −78° C., and acetone (10.0 g, 172.2 mmol) added followed by dropwise addition of i-Pr₂NEt (66.7 g, 516.5 mmol) over 30 min. Stirring was continued for 1 h then the solution was poured onto a vigorously stirred mixture of saturated NaHCO₃ (200 mL) and CH₂Cl₂ (300 mL). The organic phase was separated, washed with ice-cold 1 NH₂SO₄ (200 mL×2) and brine (200 mL), dried over Na₂SO₄ and evaporated, the residue oil was purified by distillation under reduced pressure (1 mm Hg, 78-82° C.) to give the title compound. (19.5 g, 70.7%) as a colorless oil. This was used in the next step.

### Intermediate 2: [3,3-bis(ethyloxy)-1-methylpropylidene]propanedinitrile

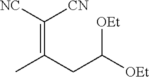

Malononitrile (22.78 g, 344.71 mmol) was added in portions over 15 min to a stirred solution of 4,4-bis(ethyloxy)-2-butanone (46.03 g, 287.31 mmol) in PhMe (250 mL) containing acetic acid (5.75 mL, 100.56 mmol) and piperidine (9.94 mL, 100.56 mmol). Stirring was continued at r.t. for overnight, and the resulting dark red solution was directly purified by distillation under reduced pressure (1 mm Hg, 108° C.) to give the crude product (45.3 g, 75.7%) as a colorless oil. This was used in the next step.

### Intermediate 3: 4-methyl-2-oxo-1,2-dihydro-3-pyridinecarbonitrile

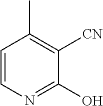

The compound [3,3-bis(ethyloxy)-1-methylpropylidene]propanedinitrile (32.21 g, 154.66 mmol) was dropwise added to a stirred solution of concentrated H₂SO₄ (48.54 g, 494.93 mmol) at a rate so that the reaction contents did not exceed 30° C. The reaction mixture was then heated to 50° C. and held at that temperature for 2 h. The reaction mixture was cooled to r.t. followed by the addition of H₂O (100 mL). The product was filtered from the reaction mixture, washed with water (30 mL) and dried to give the title compound. (20.75 g, >100%) as a yellow solid. ¹H NMR (400 MHz, DMSO): δ 2.48 (s, 3H), 6.28 (d, J=6.6 Hz, 1H), 7.63 (d, J=6.6 Hz, 1H), 12.32 (br, 1

H).

### Intermediate 4: 4-methyl-2-oxo-1,2-dihydro-3-pyridinecarboxylic acid

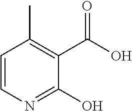

The compound 4-methyl-2-oxo-1,2-dihydro-3-pyridinecarbonitrile (2.5 g, 18.6 mmol) was added to 50 mL round bottom flask and followed by the addition of 50% H₂SO₄ (20 mL), and the mixture was stirred at 120° C. for 8 h. The reaction mixture was cooled to r.t. and H₂O (20 mL) was added, stirring was continued for 20 min, the precipitates was filter off and dried to give the desired product (1.8 g, 63.1%) as an off-white solid. ¹H NMR (400 MHz, DMSO): δ 2.60 (s, 3H), 6.55 (d, J=6.6 Hz, 1H), 7.75 (d, J=6.6 Hz, 1H), 13.05 (br, 1H).

### Intermediate 5: Methyl 4-methyl-2-oxo-1,2-dihydro-3-pyridinecarboxylate

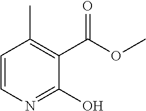

The compound 4-methyl-2-oxo-1,2-dihydro-3-pyridinecarboxylic acid (1.6 g, 10.5 mmol) was added to 100 mL round bottom flask containing of 50 mL MeOH and followed by dropwise addition of H₂SO₄ (0.6 mL), and then the mixture was stirred at 80° C. for 4 h. The solvent of MeOH was removed under reduced pressure and saturated NaHCO₃ (20 mL) was added, and then the aqueous layer was extracted by CH₂Cl₂ (30 mL×3) and dried over Na₂SO₄. The solvent was removed under reduced pressure to give the desired product (1.4 g, 80.2%) as an off-white solid. ¹H NMR (400 MHz, DMSO): δ 2.09 (s, 3H), 3.75 (s, 3H), 6.10 (d, J=6.4 Hz, 1H), 7.36 (d, J=6.4 Hz, 1H), 11.80 (br, 1H).

### Intermediate 6: Methyl 2-chloro-4-methyl-3-pyridinecarboxylate

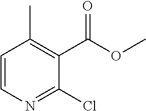

The compound Methyl 4-methyl-2-oxo-1,2-dihydro-3-pyridinecarboxylate (1.4 g, 8.4 mmol) was added to 25 mL round bottom flask containing of 8.0 mL POCl₃ and the mixture was stirred at reflux for 3.5 h. The reagent of POCl₃ was removed under reduced pressure, saturated NaHCO₃ (30 mL) was added and the aqueous layer was extracted by AcOEt (30 m L×3) and dried over Na₂SO₄. The solvent was removed under reduced pressure to give the desired product (1.5 g, 96.5%) as a colorless liquid. ¹H NMR (400 MHz, CDCl₃): δ 2.34 (s, 3H), 3.97 (s, 3H), 7.10 (d, J=5.2 Hz, 1H), 8.28 (d, J=5.2 Hz, 1H).

### Intermediate 7: 2-chloro-4-methyl-3-pyridinecarboxylic acid

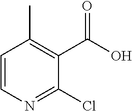

The compound Methyl 2-chloro-4-methyl-3-pyridinecarboxylate (1.3 g, 7.0 mmol) was added to 50 mL round bottom flask containing of 15 mL MeOH and followed by the addition of 20% NaOH (5 mL), and then the mixture was stirred at r.t. for 12 h. Adjust the value of pH to 4.0 and the solvent of MeOH and H₂O was remove under reduced pressure to give the crude product which was directly used in the next step.

### Intermediate 8: 1-methylethyl 2-chloro-4-methyl-3-pyridinecarboxylate

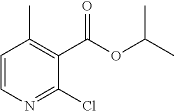

The crude product 2-chloro-4-methyl-3-pyridinecarboxylic acid (3.51 g) was added to the 100 mL round bottom flask containing of 60 mL acetone and followed by the addition of K₂CO₃ (8.48 g, 61.37 mmol) and i-Prl (10.43 g, 61.37 mmol), and then the mixture was stirred at 56° C. for overnight. The solvent of acetone was removed under reduced pressure and then 60 mL H₂O was added, and the aqueous layer was extracted by AcOEt (100 mL×3) and dried over Na₂SO₄. The solvent was removed under reduced pressure to give the crude product which was purified by column chromatography over silica gel (Petroleum Ether/EtOAc=20/1) to afford the desired product (3.4 g, two step yield: 77.8%) as a colorless liquid. ¹H NMR (400 MHz, CDCl₃): δ 1.39 (d, J=6.4 Hz, 6H), 2.35 (s, 3H), 5.33 (m, 1H), 7.09 (d, J=5.2 Hz, 1H), 8.27 (d, J=5.2 Hz, 1H). ¹³C NMR (100 MHz, CDCl₃): δ 19.3, 21.8, 70.3, 124.3, 130.8, 147.6, 149.6, 165.5.

### Intermediate 9: 2-chloro-3-iodopyridine

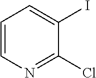

N-butyllithium (2.3 M in hexanes, 92.47 mL, 212.69 mmol) was added dropwise to 2,2,6,6-tetramethylpiperidine (31.47 g, 222.82 mmol) in tetrahydrofuran (150 mL) at −20° C. After stirred for 1 h at −20° C., the mixture was cooled to −78° C., and then 2-chloropyridine (23.00 g, 202.56 mmol) in tetrahydrofuran (150 mL) was added dropwise. After stirring for 2 h at −78° C., the mixture was treated with a solution of iodine (53.98 g, 212.69 mmol) in tetrahydrofuran (100 mL), and then water (200 mL) was added in several partitions. The mixture was extracted with diethyl ether (3×100 mL), and the combined organic layers were washed with 1.0 M hydrochloric acid (50 mL), a saturated aqueous solution of sodium thiosulfate (50 mL), brine (50 mL) and dried over Na₂SO₄. After the organic solvent was removed, the residue was recrystallized from hexane to afford the product as a white solid (35.50 g, yield 76%) ¹H NMR (400 MHz, CDCl₃): δ 6.94-6.97 (m, 1H), 8.14-8.16 (m, 1H), 8.36-8.37 (m, 1 H).

### Intermediate 10: 2-chloro-4-iodo-3-pyridinecarboxylic acid

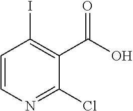 [SMILES: O=C(O)c1c(I)ccnc1Cl]

To a solution of LDA (prepared by reaction of diisopropylamine (33.38 mL, 235.21 mmol) in THF (300 mL) and n-butyllithium (95.88 mL, 2.3 M, 220.51 mmol) at −20° C. for 30 min) was added dropwise a solution of 2-chloro-3-iodopyridine (35.20 g, 147.01 mmol) in THF (300 mL) at −78° C. The mixture was stirred at the same temperature for 2 h, and then CO₂ gas was introduced for 20 min. After the reaction had been quenched by addition of water (50 mL) and 2 N HCl (200 mL), the mixture was extracted with ethyl acetate (3×200 mL). The combined extracts were dried over Na₂SO₄, filtered, and then concentrated in vacuo. The residue was recrystallized from ethyl acetate and petroleum ether to afford the product as a yellow solid (31.60 g, Yield 75.83%). ¹H NMR (400 MHz, DMSO): δ 7.98 (d, J=5.6 Hz, 1H), 8.12 (d, J=5.6 Hz, 1H).

### Intermediate 11: 1-methylethyl 2-chloro-4-iodo-3-pyridinecarboxylate

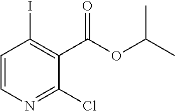

The mixture of 2-chloro-4-iodo-3-pyridinecarboxylic acid (15.17 g, 53.52 mmol), i-PrBr (19.75 g, 160.56 mmol) and K₂CO₃ (22.19 g, 160.56 mmol) in DMF (100 mL) was heated to 50° C. overnight. After cooled to room temperature, the mixture was diluted with Et₂O (500 mL), and then washed with water (50 mL×4) and brine (50 mL), dried over Na₂SO₄. The solvent was concentrated to afford the product as a yellow solid (16.90 g, Yield 97.0%). ¹H NMR (400 MHz, CDCl₃): δ 1.42 (d, J=6.0 Hz, 6H), 5.35 (q, J=6.0 Hz, 1H), 7.70 (d, J=4.8 Hz, 1H), 8.03 (d, J=4.8 Hz, 1H).

### Intermediate 12: 1-methylethyl 2-chloro-4-phenyl-3-pyridinecarboxylate

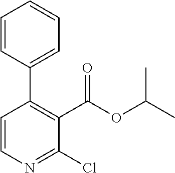 [SMILES: CC(C)OC(=O)c1c(-c2ccccc2)ccnc1Cl]

To a mixture of Pd(OAc)₂ (40.0 mg, 0.18 mmol), PPh₃ (116.8 mg, 0.45 mmol), K₂CO₃ (1.85 g, 13.36 mmol), and 1-methylethyl 2-chloro-4-iodo-3-pyridinecarboxylate (2.9 g, 8.91 mmol) in 1,4-dioxane (20 mL) was added PhB(OH)₂ (1.30 g, 10.69 mmol) and the reaction mixture was refluxed for overnight. After the solvent was evaporated, the residue was dissolved in EtOAc (100 mL), and then the solution was washed with water (30 mL), brine (30 mL), and dried over Na₂SO₄. The solvent was removed to obtain the crude product which was purified on silica gel chromatography column (Petroleum Ether: EtOAc=40:1) to afford the product (2.05 g, yield 83.6%) ¹H NMR (400 MHz, CDCl₃): δ 1.10 (d, J=6.4 Hz, 6H), 5.05-5.14 (m, 1H), 7.27 (d, J=5.2 Hz, 1H), 7.40-7.45 (m, 5H), 8.45 (d, J=5.2 Hz, 1H) ¹³C NMR (100 MHz, CDCl₃): δ 21.5, 70.3, 123.4, 128.3, 128.9, 129.0, 129.5, 137.0, 148.1, 149.9, 150.6, 165.4.

### Intermediate 13: 1-methylethyl 2-{(3R)-3-[{[(1,1-dimethylethyl)oxy]carbonyl}(ethyl)amino]-1-pyrrolidinyl}-4-iodo-3-pyridinecarboxylate

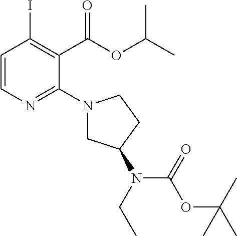 [SMILES: CCN(C(=O)OC(C)(C)C)[C@@H]1CCN(c2nccc(I)c2C(=O)OC(C)C)C1]

To the suspension of 1-methylethyl 2-chloro-4-iodo-3-pyridinecarboxylate (3.0 g, 9.22 mmol) and K₂CO₃ (1.91 g, 13.82 mmol) in DMF (50 mL), S2-1 (QJ208951-120) (2.07 g, 9.68 mmol) was added. The reaction mixture was stirred and heated at 80° C. overnight. After cooling to r.t., the solvent was removed and the residue was purified on silica gel chromatography column (PE: EA=8:1) to obtain 1.60 g of UMS-109-1-1 as oil and 2.47 g of UMS-109-1-2 as solid. ¹H NMR (400 MHz, CDCl₃): δ 1.12 (t, J=6.8 Hz, 3H), 1.40 (d, J=5.2 Hz, 3H), 1.44 (d, J=4.0 Hz, 3H), 1.46 (s, 9H), 2.04-2.10 (m, 2H), 3.10-3.63 (m, 6H), 4.51-4.66 (m, 1H), 5.24-5.27 (m, 1H), 7.05 (d, J=4.8 Hz, 1H), 7.73 (d, J=4.8 Hz, 1H). ¹³C NMR (100 MHz, CDCl₃): δ 15.7, 21.9, 28.7, 28.9, 38.6, 46.9, 50.3, 54.7, 70.6, 80.0, 106.4, 120.1, 122.7, 128.7, 148.4, 153.7, 168.4.

### Intermediate 14: 1-methylethyl 4-iodo-2-[4-(phenylmethyl)-1-piperazinyl]-3-pyridine carboxylate

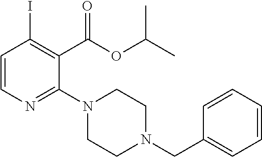 [SMILES: CC(C)OC(=O)c1c(I)ccnc1N1CCN(Cc2ccccc2)CC1]

The mixture of 1-methylethyl 2-chloro-4-iodo-3-pyridinecarboxylate (16.30 g, 50.07 mmol), 1-benzylpiperazine dihydrochloride (13.72 g, 55.08 mmol) and K₂CO₃ (13.84 g, 100.14 mmol) in DMF (100 mL) was heated to 80° C. overnight. After cooled to room temperature, the reaction was diluted with EtOAc (500 mL), and then washed with water (50 mL×4) and brine (50 mL), dried over Na₂SO₄. After the solvent was removed, the residue was purified on si-gel column chromatography, eluting with Petroleum Ether/EtOAc (5:1). 3.2 g of the product was obtained as a yellow solid (yield 13.73%) ¹H NMR (400 MHz, CDCl₃): δ 1.40 (d, J=5.6 Hz, 6H), 2.51 (t, J=5.2 Hz, 4H), 3.39 (t, J=5.2 Hz, 4H), 3.54 (s, 2H), 5.22-5.28 (m, 1H), 7.24-7.34 (m, 6H), 7.80 (d, J=5.2 Hz, 1H); ¹³C NMR (100 MHz, CDCl₃) δ 21.9, 49.1, 53.1, 63.1, 70.2, 106.1, 126.0, 127.3, 128.4, 129.3, 138.0, 148.2, 158.3, 167.8.

### Intermediate 15: 1-methylethyl 2-(1-piperazinyl)-3-pyridinecarboxylate

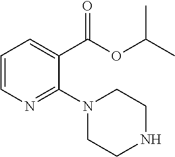 [SMILES: CC(C)OC(=O)c1cccnc1N1CCNCC1]

Piperazine (30.2 g, 351 mmol) and DIPEA (70 mL, 401 mmol) was added to a 500 mL three-neck round flask with DMF (200 mL), the solution was heated to 100° C., dropwise addition of 1-methylethyl 2-chloro-3-pyridinecarboxylate (20.0 g, 100 mmol) to the solution. The resulting mixture was continued stirring at 100° C. for 30 min, and then cooled to room temperature. The solvent was removed under reduced to give the crude product, which was purified by flash chromatography, eluting at 1% Et₃N of Heptane over 20 min and obtained the desired product (19.1 g, 76.0%). LC/MS: m/z=249.9[M+H]⁺, Ret. Time: 0.59 min.

### Intermediate 16: 1-methylethyl 2-{4-[(2-bromophenyl)methyl]-1-piperazinyl}-3-pyridinecarboxylate

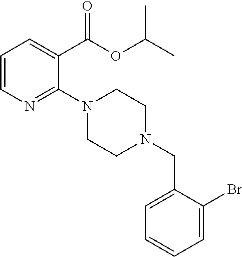 [SMILES: CC(C)OC(=O)c1cccnc1N1CCN(Cc2ccccc2Br)CC1]

To a solution of 1-methylethyl 2-(1-piperazinyl)-3-pyridinecarboxylate (750 mg, 3.01 mmol) in methanol (8.0 mL) with acetic acid (18.07 mg, 0.30 mmol), added 2-bromobenzaldehyde (835 mg, 4.51 mmol). The solution was stirred for 4 h at room temperature. Then MP-cyanoborohydride (3.37 g, 7.52 mmol) was added, the resulting mixture was stirred at room temperature for 12 h. The polymer was filtered and washed with AcOEt, the filtrate was concentrated to give crude product which was purified on a silica cartridge (4 g) with a Combiflash Companion, eluting with 5-15% EtOAc/Hexane, 365 mg desired compound was obtained (yield: 29.0%).

LC/MS: m/z=418.2[M+H]⁺, Ret. Time: 0.88 min.

### Intermediate 17: 1-methylethyl 2-{4-[3-bromophenyl)methyl]-1-piperazinyl}-3-pyridinecarboxylate

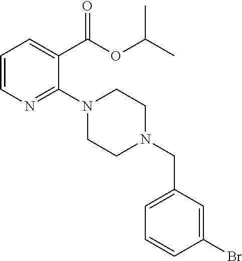 [SMILES: CC(C)OC(=O)c1cccnc1N1CCN(Cc2cccc(Br)c2)CC1]

To a solution of 1-methylethyl 2-(1-piperazinyl)-3-pyridinecarboxylate (750 mg, 3.01 mmol) in methanol (8.0 mL) with acetic acid (18.07 mg, 0.30 mmol), added 3-bromobenzaldehyde (835 mg, 4.51 mmol). The solution was stirred for 4 h at room temperature. Then MP-cyanoborohydride (3.37 g, 7.52 mmol) was added, the resulting mixture was stirred at room temperature for 12 h. The polymer was filtered and washed with AcOEt, the filtrate was concentrated to give crude product which was purified on a silica cartridge (4 g) with a Combiflash Companion, eluting with 5-15% EtOAc/Hexane, 665 mg desired compound was obtained (yield: 52.8%).

LC/MS: m/z=418.2[M+H]⁺, Ret. Time: 0.88 min.

### Intermediate 18: 1-methylethyl 2-{4-[(4-bromophenyl)methyl]-1-piperazinyl}-3-pyridinecarboxylate

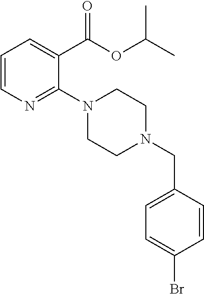 [SMILES: CC(C)OC(=O)c1cccnc1N1CCN(Cc2ccc(Br)cc2)CC1]

To a solution of 1-methylethyl 2-(1-piperazinyl)-3-pyridinecarboxylate (750 mg, 3.01 mmol) in methanol (8.0 mL) with acetic acid (18.07 mg, 0.30), added 3-bromobenzaldehyde (835 mg, 4.51 mmol). The solution was stirred for 4 h at room temperature. Then MP-cyanoborohydride (3.34 g, 7.52 mmol) was added, the resulting mixture was stirred at room temperature for 12 h. The polymer was filtered and washed with AcOEt, the filtrate was concentrated to give crude product which was purified on a silica cartridge (4 g) with a Combiflash Companion, eluting with 5-15% EtOAc/Hexane, 682 mg desired compound was obtained (yield: 54.2%).

LC/MS: m/z=418.2[M+H]⁺, Ret. Time: 0.91 min.

### Intermediate 19: 1-methylethyl 4-phenyl-2-(1-piperazinyl)-3-pyridinecarboxylate

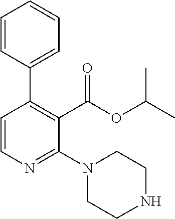 [SMILES: CC(C)OC(=O)c1c(-c2ccccc2)ccnc1N1CCNCC1]

Piperazine (3.5 g, 19.04 mmol) and DIPEA (3.8 mL) was added to a 100 mL three-neck round flask with DMF (80 mL), the solution was heated to 100° C., dropwise addition of 2-chloro-4-phenyl-3-pyridinecarboxylate (1.5 g, 5.44 mmol) to the solution. The resulting mixture was continued stirring at 110° C. for 4 hr, and then cooled to room temperature. The solvent was removed under reduced to give the crude product, which was purified by flash chromatography, eluting at 1% Et3N of Heptane over 20 min and obtained the desired product (1.4 g, 79.0%).

LC/MS: m/z=326.0[M+H]⁺, Ret. Time: 0.83 min.

### Intermediate 20:1-methylethyl 2-{(3R)-3-[{[(1,1-dimethylethyl)oxy]carbonyl}(ethyl)amino]-1-pyrrolidinyl}-4-phenyl-3-pyridinecarboxylate

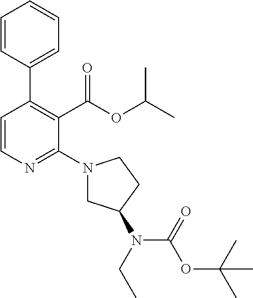 [SMILES: CCN(C(=O)OC(C)(C)C)[C@@H]1CCN(c2nccc(-c3ccccc3)c2C(=O)OC(C)C)C1]

1-methylethyl 2-{(3R)-3-[{[(1,1-dimethylethyl)oxy]carbonyl}ethyl)amino]-1-pyrrolidin-yl}-4-iodo-3-pyridinecarboxylate (975 mg, 1.93 mmol) and phenylboronic acid (283 mg, 2.324 mmol) were suspended in 1,4-Dioxane (12.0 mL) and H₂O (4.0 mL) in a 20 mL microwave vial. Potassium carbonate (944 mg, 6.78 mmol) and tetrakis(triphenylphosphine)palladium (134 mg, 0.116 mmol) was added, and then heated in Biotage microwave at 130° C. in high setting for 20 min. Filtered and concentrated the solvent to give the crude product, which was purified on a silica cartridge (12 g) with a Combiflash Companion, eluting with 10% AcOEt/Hexane, 822.4 mg desired compound was obtained (yield: 90%).

LC/MS: m/z=454.1[M+H]⁺, Ret. Time: 1.15 min.

### Intermediate 21: 1-methylethyl 2-[(3R)-3-(ethylamino)-1-pyrrolidinyl]-4-phenyl-3-pyridinecarboxylate

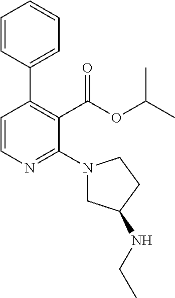 [SMILES: CCN[C@@H]1CCN(c2nccc(-c3ccccc3)c2C(=O)OC(C)C)C1]

1-methylethyl 2-{(3R)-3-[{[(1,1-dimethylethyl)oxy]carbonyl}(ethyl)amino]-1-pyrrolidinyl}-4-phenyl-3-pyridinecarboxylate (822.4 mg, 1.81 mmol) was taken up in a solution of 2 M HCl in dioxane (1.8 mL, 3.60 mmol). The reaction was stirred at room temperature for 12 hr. Added saturated NaHCO₃ (5 mL) and stirred for 20 min, concentrated and added DCM (10 mL), filtered with 500 mg Spe-ed NH₂-Column and concentrated the filtrate to give the free base (602 mg, 94%).

LC/MS: m/z=354.0[M+H]⁺, Ret. Time: 0.81 min.

### Intermediate 22: 1-methylethyl 4-methyl-2-(1-piperazinyl)-3-pyridinecarboxylate

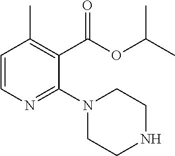 [SMILES: Cc1ccnc(N2CCNCC2)c1C(=O)OC(C)C]

Piperazine (1.41 g, 16.38 mmol) and DI PEA (3.02 g, 23.40 mmol) was added to a 100 mL three-neck round flask with DMF (60 mL), the solution was heated to 120° C., dropwise addition of 1-methylethyl 2-chloro-4-methyl-3-pyridinecarboxylate (1.0 g, 4.68 mmol) in DMF (20 mL) to the solution. The resulting mixture was continued stirring at 120° C. for 4 hr, and then cooled to room temperature. The solvent was removed under reduced to give the crude product, which was purified by flash chromatography, eluting at 5% AcOEt of Heptane with 1% Et₃N over 20 min and concentrated to give the desired product (720 mg, 58.4%). LC/MS: m/z=263.9[M+H]⁺, Ret. Time: 0.87 min.

### Intermediate 23: 1-methylethyl 2-[4-({4-[(ethylamino)methyl]phenyl}methyl)-1-piperazinyl]-3-pyridinecarboxylate

### Preparation 1

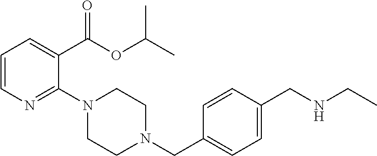 [SMILES: CCNCc1ccc(CN2CCN(c3ncccc3C(=O)OC(C)C)CC2)cc1]

To a solution of 1-methylethyl 2-{4-[(4-formylphenyl)methyl]-1-piperazinyl}-3-pyridinecarboxylate (480 mg, 1.31 mmol) in methanol (10 mL) was added 2 M ethylamine (1.3 mL, 2.61 mmol), and added acetic acid (15.69 mg, 20 mol %), the stirring was kept for 4 hours at room temperature. Sodium cyanoborohydride (287 mg, 4.57 mmol) was added and kept stirring at room temperature for 16 hr. Removed the solvent of methanol and got the crude product, which was purified on a silica cartridge (4 g) with a Combiflash Companion, eluting at 5% DCM of AcOEt over 20 min, and obtained the product (210 mg, 40.5%).

LC/MS: m/z=397.1[M+H]⁺, Ret. Time: 0.73 min.

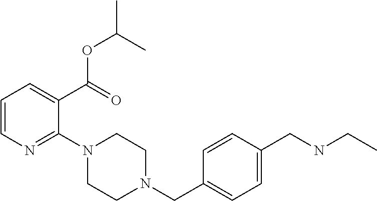 [SMILES: CCNCc1ccc(CN2CCN(c3ncccc3C(=O)OC(C)C)CC2)cc1]

### Preparation 2

1-Methylethyl2-{4-[(4-formylphenyl)methyl]-1-piperazinyl}-3-pyridinecarboxylate (2.94 g, 8.00 mmol), was dissolved in Tetrahydrofuran (THF) (23.54 ml), cooled to 4° C. on an ice bath and ethylamine 2M in THF (8.00 ml, 16.00 mmol), and acetic acid (0.458 ml, 8.00 mmol) were added. Stirred for 20 min and then sodium triacetoxyborohydride (2.54 g, 12.00 mmol) was added in several portions. Stirred on the ice bath for 20 min more and then slowly warmed to 23° C. and stirred 16 h and then diluted with EtOAc (300 mL) and washed with 1 M aq NaOH (100 ml). The aq was back extracted with EtOAc (100 mL) and the combined EtOAc was washed with 1 M aq NaOH (100 mL), water (100 mL) and satd aq NaCl (100 mL) and dried (Na₂SO₄) and concentrated to afford a brown oil which was purified on a silica cartridge (120 g) eluting at 85 mL/min with a gradient running from dichloromethane to 10% MeOH containing 2M NH₃ in dichloromethane over 35 min. The desired fractions were pooled and concentrated to afford the title compound (2.74 g, 6.91 mmol, 86% yield) as a yellow oil. Lcms rt 0.70[M+H]=397.1.

There was no detectable dimer which resulted from reaction at 23° C., and NaCNBH₃ as the reducing agent.

### Intermediate 24: Isopropyl 2-chloronicotinate

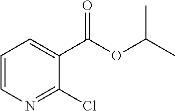

To a solution of 2-chloronicotinic acid (40 g, 254 mmol) in CH₂Cl₂ (150 mL) was added oxalyl chloride (96.7 g, 762 mmol) dropwise at 0° C. followed by DMF (1 mL), the resulting solution was stirred at room temperature for 2 h. The solvent was evaporated to dryness. The residue was dissolved in CH₂Cl₂ (100 mL), which was added dropwise to a mixture of isopropanol (30.5 g, 508 mmol) and Et₃N (106 mL, 762 mmol) at 0° C. The reaction mixture was stirred at room temperature for 16 h, and then was poured into ice-water, CH₂Cl₂ (3×100 mL). The combined organic layer was washed with brine, dried over MgSO₄, concentrated giving the crude product which was purified by column chromatography on silica gel (200-300 um, 500 g) eluting by 10% ethyl acetate of petroleum ether. The desired fraction was concentrated to obtain the title compound 45 g (yield: 89%) as yellow oil.

LC/MS: M/z=200.1 (M+H), Ret. Time: 1.42 min.

Intermediate 25: (R)-Isopropyl 2-[3-(ter-butoxycarbonylamino)pyrrolidine-1-yl]nicotinate

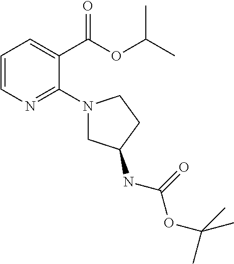 [SMILES: CC(C)OC(=O)c1cccnc1N1CC[C@@H](NC(=O)OC(C)(C)C)C1]

To a solution isopropyl 2-chloronicotinate (500 mg, 2.5 mmol) in THF (10 mL) was added (R)-3-Boc-aminopyrrolidine (558 mg, 3 mmol) followed by Et₃N (500 mg, 5 mmol). The resulting mixture was stirred at reflux for 3 h. The solvent was evaporated under reduced pressure and the residue was purified by column chromatography on silica gel (200-300 um, 50 g) eluting with 10% to 25% EtOAc of n-Hexane to obtain the title compound 790 mg (yield: 91%) as yellow oil.

LC/MS: M/z=350.2 (M+H), Ret. Time: 1.48 min.

Intermediate 26: (R)-Isopropyl 2-{3-[tert-butoxycarbonyl(ethyl)amino]pyrrolidin-1-yl}nicotinate

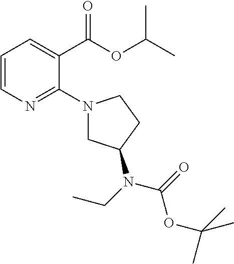 [SMILES: CCN(C(=O)OC(C)(C)C)[C@@H]1CCN(c2ncccc2C(=O)OC(C)C)C1]

To a solution (R)-isopropyl 2-[3-(ter-butoxycarbonylamino)pyrrolidine-1-yl]nicotinate (790 mg, 2.26 mmol) in DMF (5 mL) was added NaH (180 mg, 4.52 mmol). After stirring for 20 min, bromoethane (741 mg, 6.8 mmol) was added dropwise via syringe. The resulting mixture was stirred at room temperature for 5 h. Water (10 mL) was added, the mixture was extracted with ethyl acetate (3×50 mL). The combined organic phase was washed with brine (3×50 mL), dried (Na₂SO₄), concentrated giving the crude product which was purified by column chromatography on silica gel (200-300 um, 80 g) eluting with 10% to 25% EtOAc of n-Hexane to obtain the title compound 660 mg (yield: 77%) as yellow oil.

LC/MS: M/z=378.0 (M+H), Ret. Time: 1.89 min.

### Intermediate 27: 1-Methylethyl 2-[(3S)-3-({[(1,1-dimethylethyl)oxy]carbonyl}amino)-1-pyrrolidinyl]-3-pyridinecarboxylate

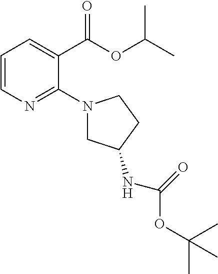 [SMILES: CC(C)OC(=O)c1cccnc1N1CC[C@H](NC(=O)OC(C)(C)C)C1]

To a solution of isopropyl 2-chloronicotinate (2 g, 10 mmol) in THF (50 mL) was added (S)-tert-butyl pyrrolidin-3-ylcarbamate (1.866 g, 10 mmol) and Et₃N (1.2 g, 12 mmol). The mixture was heated to reflux overnight. Solvent was removed. Water (50 mL) was added and extracted with EtOAc (3×50 mL). The combined organic layer was dried over Na₂SO₄ and concentrated to obtain the title compound (3.5 g, 100%) as yellow oil. LC-MS m/z 350 (M+H)⁺, 1.75 min (ret time)

### Intermediate 28: 1-Methylethyl 2-[(3S)-3-amino-1-pyrrolidinyl]-3-pyridinecarboxylate

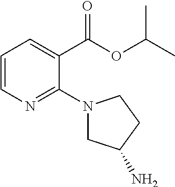 [SMILES: CC(C)OC(=O)c1cccnc1N1CC[C@H](N)C1]

To a solution of 1-Methylethyl 2-[(3S)-3-({[(1,1-dimethylethyl)oxy]carbonyl}amino)-1-pyrrolidinyl]-3-pyridinecarboxylate (1.1 g, 3 mmol) in dichloromethane (50 mL) was added CF₃COOH (3 mL). The mixture was stirred at room temperature for 2 h. Saturated NaHCO₃ was added and extracted with dichloromethane twice, washed with saturated NaCl. The combined organic layer was dried over Na₂SO₄ and concentrated to obtain the title compound (772 mg, 98%) as yellow oil. LC-MS m/z 250 (M+H)⁺, 1.37 min (ret time)

### Intermediate 29: 1-Methylethyl 2-{(3S)-3-[{[(1,1-dimethylethyl)oxy]carbonyl}(methyl)amino]-1-pyrrolidinyl}-3-pyridinecarboxylate

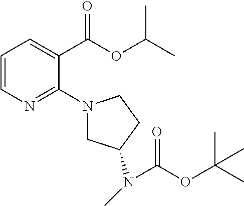 [SMILES: CC(C)OC(=O)c1cccnc1N1CC[C@H](N(C)C(=O)OC(C)(C)C)C1]

To a solution of 1-Methylethyl 2-[(3S)-3-({[(1,1-dimethylethyl)oxy]carbonyl}amino)-1-pyrrolidinyl]-3-pyridinecarboxylate (1 g, 2.87 mmol) in DMF (10 mL) was added NaH (60%, 138 mg, 3.44 mmol). The mixture was stirred at room temperature for 5 min. CH₃I (407 mg, 2.87 mmol) was added. The reaction mixture was continuously to stir for 2 h. Water was added and extracted with EtOAc. The combined organic layer was washed with brine, concentrated to obtain the title compound (1 g, 96%) as yellow oil.

LC-MS m/z 364(M+H)⁺, 1.83 min (ret time)

### Intermediate 30:1-Methylethyl 2-[(3S)-3-(methylamino)-1-pyrrolidinyl]-3-pyridinecarboxylate

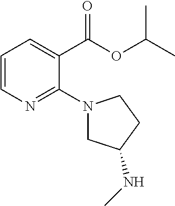 [SMILES: CN[C@H]1CCN(c2ncccc2C(=O)OC(C)C)C1]

To a solution of 1-Methylethyl 2-{(3S)-3-[{[(1,1-dimethylethyl)oxy]carbonyl}(methyl)amino]-1-pyrrolidinyl}-3-pyridinecarboxylate (1 g, 2.8 mmol) in dichloromethane (50 mL) was added CF₃COOH (3 mL). The mixture was stirred at room temperature for 2 h. Saturated NaHCO₃ was added, extracted with dichloromethane twice. The combined organic layer was washed with saturated NaCl, dried over Na₂SO₄ and concentrated to obtain the title compound (670 mg, 92%) as yellow oil. LC-MS m/z 264 (M+H)⁺, 1.42 min (ret time)

### Intermediate 31: 1-Methylethyl 2-{(3S)-3-[{[(1,1-dimethylethyl)oxy]carbonyl}(ethyl)amino]-1-pyrrolidinyl}-3-pyridinecarboxylate

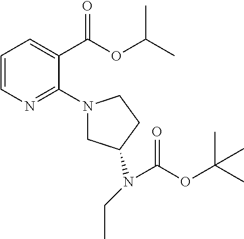 [SMILES: CCN(C(=O)OC(C)(C)C)[C@H]1CCN(c2ncccc2C(=O)OC(C)C)C1]

To a solution of 1-Methylethyl 2-[(3S)-3-({[(1,1-dimethylethyl)oxy]carbonyl}amino)-1-pyrrolidinyl]-3-pyridinecarboxylate (1 g, 2.87 mmol) in DMF (10 mL) at room temperature was added NaH (60%, 138 mg, 3.44 mmol). CH₃CH₂I (447 mg, 2.87 mmol) was added after 5 min. The reaction mixture was stirred for 2 h. Water was added and extracted with EtOAc. The combined organic layer was washed with brine, concentrated to obtain the title compound (1 g, 93%) as yellow oil. LC-MS m/z 378 (M+H)⁺, 1.88 min (ret time)

### Intermediate 32: 1-Methylethyl 2-[(3S)-3-(ethylamino)-1-pyrrolidinyl]-3-pyridinecarboxylate

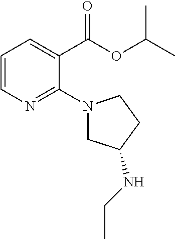 [SMILES: CCN[C@H]1CCN(c2ncccc2C(=O)OC(C)C)C1]

To a solution of 1-Methylethyl 2-{(3S)-3-[{[(1,1-dimethylethyl)oxy]carbonyl}(ethyl)amino]-1-pyrrolidinyl}-3-pyridinecarboxylate (1 g, 2.7 mmol) in dichloromethane (50 mL) was added CF₃COOH (3 mL). The mixture was stirred at room temperature for 2 h. Saturated NaHCO₃ was added. It was extracted with dichloromethane twice. The combined organic layer was washed with saturated NaCl, dried over Na₂SO₄ and concentrated to obtain the title compound (720 mg, 98%) as yellow oil. LC-MS m/z 278 (M+H)⁺, 1.48 min (ret time)

Intermediate 33: (3-Bromophenyl)(phenyl)methanol

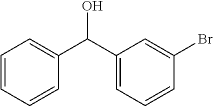 [SMILES: OC(c1ccccc1)c1cccc(Br)c1]

To a mixture of Mg (2.5 g, 104.17 mmol) in THF (50 mL) was added iodine (one drop) under Argon. PhBr (16.36 g, 104.17 mmol) in THF (50 mL) was added dropwise and kept slightly boiling. After complete addition, the gray mixture was stirred at reflux for 1 h. The above solution was added to a mixture of 3-bromobenzaldehyde (19.27 g, 104.17 mmol) dropwise under Argon, the resulting mixture was stirred at room temperature for 2 h and then quenched with water (20 mL). The solution was extracted with EtOAc (3×150 mL), the combined organics were washed with brine (2×50 mL), dried, concentrated to give the crude product which was purified via column chromatography (200-300 um silica gel, 100 g, elution with 5% EtOAc in hexane) to give the desired product (23.5 g, 86%) as pale yellow oil. LC-MS m/z 246.9 (M-18+H)⁺, 1.55 min (ret time)

### Intermediate 34: (4-Bromophenyl)(phenyl)methanol

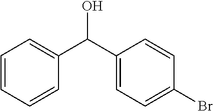 [SMILES: OC(c1ccccc1)c1ccc(Br)cc1]

Following the general procedure of (3-Bromophenyl)(phenyl)methanol

Mg (0.25 g, 10.42 mmol), PhBr (1.64 g, 10.42 mmol) and 4-bromobenzaldehyde (19.27 g, 104.17 mmol) in THF (50 mL) were reacted to give the title compound (2.5 g, 91%) as yellow solid. LC-MS m/z 244.9 (M−18+H)⁺, 1.59 min (ret time)

### Intermediate 35: (2E)-3-(4-Bromophenyl)-1-phenyl-2-propen-1-one

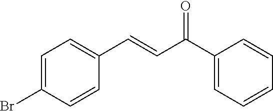 [SMILES: O=C(/C=C/c1ccc(Br)cc1)c1ccccc1]

A mixture of acetophenone (3.25 g, 27 mmol) and 4-bromobenzaldehyde (5 g, 27 mmol) was stirred in EtOH (50 mL) at room temperature. KOH (1.51 g, 27 mmol) in water (10 mL) was added dropwise. The resulting mixture was stirred at room temperature for 2 h. The mixture was poured into ice-water, the solid was collected via filtration, washed with EtOH/H₂O (1:1, 2×5 mL), dried in vacuo to give the title product (7.5 g, 97%) as pale yellow solid. LC-MS m/z 286.9 (M+H)⁺, 1.73 min (ret time).

### Intermediate 36: 1-Bromo-3-(phenylmethyl)benzene

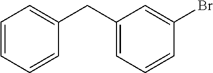 [SMILES: Brc1cccc(Cc2ccccc2)c1]

To a solution of (3-bromophenyl)(phenyl)methanol (10 g, 38 mmol) in diethyl ether was added TFA (2 mL) and the resulting solution was stirred at room temperature for 24 h. 10% NaOH (20 mL) was added, the mixture was extracted with EtOAc (3×20 mL). The combined organics were dried, concentrated to give the desired product (9.4 g, 100%) as yellow oil.

### Intermediate 37: 1-Bromo-4-(phenylmethyl)benzene

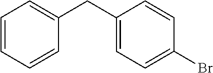 [SMILES: Brc1ccc(Cc2ccccc2)cc1]

To a solution of (4-bromophenyl)(phenyl)methanol (2.5 g, 9.5 mmol) in DCM (5 mL) was added TFA (4.6 g, 47.5 mmol), followed by Et₃SiH (3.3 g, 28.5 mmol), the resulting mixture was stirred at room temperature for 2 h. Solvent was evaporated to dryness to give the title compound (2.35 g, 100%) as yellow oil.

### Intermediate 38: 1-Bromo-4-(3-phenylpropyl)benzene

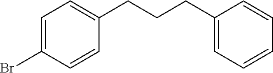 [SMILES: Brc1ccc(CCCc2ccccc2)cc1]

To a solution of 3-(4-bromophenyl)-1-phenylprop-2-en-1-one (7.5 g, 26.12 mmol) and Et₃SiH (5 mL) in DCM (30 mL) was added TFA (15 mL) dropwise. The resulting mixture was heated at 50° C. for 16 h. Solvent was evaporated to dryness, the residue was purified via flash chromatography eluting with hexane to give the title compound (6.96 g, 97%) as yellow oil. ¹H NMR (400 MHz, CDCl₃) δ 7.43-7.41 (m, 2H), 7.31-7.28 (m, 2H), 7.22-7.19 (m, 3H), 7.08 (d, J=4.0 Hz, 2H), 2.69-2.61 (m, 4H, 1.98-1.95 (m, 2H).

### Intermediate 39: 3-(Phenylmethyl)benzaldehyde

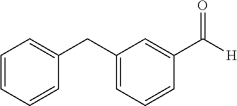 [SMILES: O=Cc1cccc(Cc2ccccc2)c1]

To a solution of 1-benzyl-3-bromobenzene (2 g, 8.1 mmol) in dry THF (5 mL) under Argon at −78° C. was added nBuLi (2.5 M, 3.5 mL, 8.75 mmol) dropwise, the resulting solution was stirred at −78° C. for 30 min. DMF (849 mg, 8.75 mmol) was added dropwise, the mixture was allowed to warm to room temperature. The mixture was poured into water (10 mL), extracted with EtOAc (3×20 mL). The combined organics were washed with brine (2×30 mL), dried over MgSO₄ and filtered. The filtrate was concentrated to give the desired product (1.58 g, 99%) as yellow oil. ¹H NMR (400 MHz, CDCl₃) δ 10.01 (s, 1H), 7.76-7.21 (m, 9H), 4.09 (s, 2H).

### Intermediate 40: 4-(Phenylmethyl)benzaldehyde

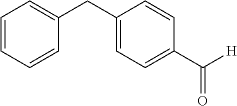 [SMILES: O=Cc1ccc(Cc2ccccc2)cc1]

Following the general procedure of 3-(Phenylmethyl)benzaldehyde 1-Benzyl-4-bromobenzene (2 g, 8.1 mmol), nBuLi (2.5 M, 3.5 mL, 8.75 mmol) and DMF (849 mg, 8.75 mmol) in THF (5 mL) were reacted to give the title compound (1.59 g, 100%) as yellow oil. ¹H NMR (400 MHz, CDCl₃) δ 10.00 (s, 1H), 7.84-7.20 (m, 9H), 4.09 (s, 2H).

### Intermediate 41: 4-(3-Phenylpropyl)benzaldehyde

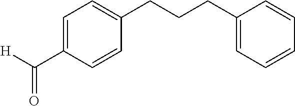 [SMILES: O=Cc1ccc(CCCc2ccccc2)cc1]

Following the general procedure of 3-(Phenylmethyl)benzaldehyde 1-Bromo-4-(3-phenylpropyl)benzene (2 g, 6.9 mmol), nBuLi (2.5 M, 3.0 mL, 7.6 mmol) and DMF (738 mg, 7.6 mmol) in THF (5 mL) were reacted to give the title compound (1.55 g, 100%) as yellow oil. ¹H NMR (400 MHz, CDCl₃) δ 10.01 (s, 1H), 7.84-7.82 (m, 2H), 7.36-7.20 (m, 7H), 2.78-2.68 (m, 4H), 2.05-2.00 (m, 2H).

### Intermediate 42: 3-(Phenylthio)benzaldehyde

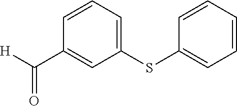 [SMILES: O=Cc1cccc(Sc2ccccc2)c1]

Cu(I)I (16.5 mg, 0.086 mmol), potassium carbonate (475 mg, 3.44 mmol) and 3-iodobenzaldehyde (400 mg, 1.72 mmol) were added to a screw-capped test tube. The tube was evacuated and backfilled with argon (3 cycles). 2-Propanol (2 mL), ethylene glycol (3.44 mmol, 200 mg) and thiophenol (190 mg, 1.72 mmol) were added via syringe at room temperature. The resulting mixture was heated at 80° C. for 20 h. The reaction was quenched by addition of water (10 mL). The mixture was extracted with EtOAc (3×50 mL). The combined extracts were washed with brine (2×30 mL), dried and concentrated to give the crude product (302 mg, 82%) as yellow oil.

### Intermediate 43: 3-Bromophenyl phenylmethyl sulfide

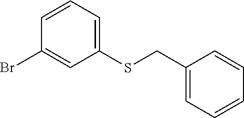 [SMILES: Brc1cccc(SCc2ccccc2)c1]

To a solution of 3-bromobenzenethiol (2 g, 10.58 mmol) in acetone (20 mL) was added

K₂CO₃ (2.2 g, 15.87 mmol) followed by BnBr (1.88 g, 11 mmol). The resulting mixture was stirred at reflux for 2 h. The mixture was filtered; the filtrate was concentrate to give the crude product (2.96 g, 100%) as yellow oil, which was used in next step without further purification. ¹H NMR (400 MHz, DMSO-d₆) δ 7.52 (s, 1H), 7.51-7.21 (m, 8H), 4.29 (s, 1H).

### Intermediate 44: 3-[(Phenylmethyl)thio]benzaldehyde

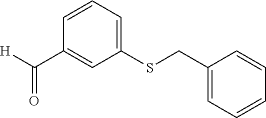 [SMILES: O=Cc1cccc(SCc2ccccc2)c1]

Following the general procedure of 3-(Phenylmethyl)benzaldehyde Benzyl(3-bromophenyl)sulfane (1 g, 3.58 mmol), nBuLi (2.5 M, 1.6 mL, 3.9 mmol) and DMF (427 mg, 5.8 mmol) in THF were reacted to give the title compound (817 mg, 100%) as yellow oil. ¹H NMR (400 MHz, CDCl₃) δ 9.96 (s, 1H), 7.81-7.22 (m, 9H), 4.20 (s, 2H).

### Intermediate 45: 1-Methylethyl 2-{4-[4-mercaptophenyl)methyl]-1-piperazinyl}-3-pyridine carboxylate

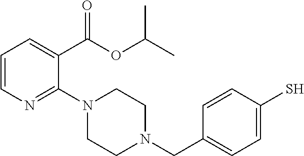 [SMILES: CC(C)OC(=O)c1cccnc1N1CCN(Cc2ccc(S)cc2)CC1]

To a solution of 1-methylethyl 2-(1-piperazinyl)-3-pyridinecarboxylate hydrochloride (200 mg, 0.70 mmol) in EtOAc (10 mL) was added Et₃N (106 mg, 1.05 mmol). The mixture was stirred at room temperature for 30 min and filtered. The filtrate was concentrated to dryness to give the free amine. The above residue was dissolved in DCE (20 mL), 4-mercaptobenzaldehyde (116 mg, 0.84 mmol) and HOAc (two drops) was added. The mixture was stirred at room temperature for 20 min. NaBH(OAc)₃ (297 mg, 1.4 mmol) was added in one portion, the resulting mixture was stirred at room temperature for 16 h. 10% NaOH (aq. 10 mL) was added, the mixture was extracted with DCM (3×150 mL). The combined organics were washed with brine (2×30 mL), dried over MgSO₄, filtered, concentrated to give the crude product which was purified via chromatography on silica gel (200-300 um, 10 g, elution with 25% EtOAc of hexane) to afford the title compound (215 mg, 83%) as purple oil. LC-MS m/z 372.0 (M+H)⁺, 1.085 min (ret time).

### Intermediate 46: 1,1-Dimethylethyl [(3R)-1-(2-methylpropanoyl)-3-pyrrolidinyl]carbamate

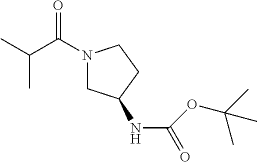 [SMILES: CC(C)C(=O)N1CC[C@@H](NC(=O)OC(C)(C)C)C1]

To a solution of isobutyric acid (500 mg, 5.68 mmol) in DMF (6 mL) at room temperature was added solid N,N′-carbonyldiimidazole (1012 mg, 6.24 mmol) portion wise during 5 min. The reaction mixture was stirred at 50° C. for 30 min. (3R)-(+)-(tert-butoxycarbonylamino)pyrrolidine (1057 mg, 5.68 mmol) was added in one portion and the resulting solution was stirred at 50° C. for 1 h. Solvent was evaporated to dryness in vacuum and the residue was dissolved in EtOAc (100 mL), washed with saturated sodium carbonate (2×30 mL), brine (2×30 mL), dried over magnesium sulphate, concentrated to give the title compound (1.45 g, 95%) as a white solid. LC-MS m/z 257.1 (M+H)⁺, 1.25 min (ret time).

### Intermediate 47: 1,1-Dimethylethyl ethyl[(3R)-1-(2-methylpropanoyl)-3-pyrrolidinyl]carbamate

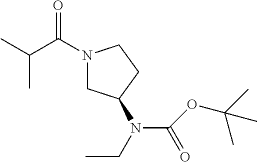 [SMILES: CCN(C(=O)OC(C)(C)C)[C@@H]1CCN(C(=O)C(C)C)C1]

To a solution of 1,1-Dimethylethyl [(3R)-1-(2-methylpropanoyl)-3-pyrrolidinyl]carbamate (700 mg, 2.73 mmol) in DMF (4 mL) was added sodium hydride (164 mg, 4.10 mmol) portion wise. After 5 min, bromoethane (0.306 mL, 4.10 mmol) was added dropwise. The resulting mixture was stirred at room temperature for 1 h. Water (5 mL) was added, the mixture was extracted with EtOAc (3×30 mL). The combined organic layer was washed with saturated brine (2×30 mL), dried over MgSO₄ and evaporated in vacuo to give the title compound (770 mg, 81%) as yellow oil. LC-MS m/z 285.1 (M+H)⁺, 1.41 min (ret time)

### Intermediate 48:(3R)—N-Ethyl-1-(2-methylpropanoyl)-3-pyrrolidinamine hydrochloride

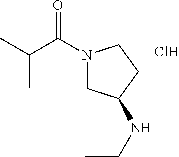

To a solution of 1,1-Dimethylethyl ethyl[(3R)-1-(2-methylpropanoyl)-3-pyrrolidinyl]carbamate (770 mg, 2.71 mmol) in methanol (20 mL) at 0° C. was added acetyl chloride (1.925 mL, 27.1 mmol) dropwise. The reaction mixture was stirred at 0° C. for 30 min. Solvent was evaporated to dryness under reduced pressure and the residue was stirred in EtOAc (20 mL) at RT for 30 min. The solid was filtered, washed with EtOAc (2 mL), dried in vacuo to give the title compound (598 mg, 100%) as white solid. LC-MS m/z 185.1 (M+H)⁺, 0.34 min (ret time).

### Intermediate 49: 1,1-Dimethylethyl 4-(3-{[(1-methylethyl)oxy]carbonyl}-2-pyridinyl)-1-piperazine carboxylate

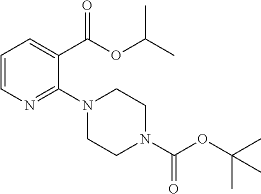 [SMILES: CC(C)OC(=O)c1cccnc1N1CCN(C(=O)OC(C)(C)C)CC1]

The solution of isopropyl 2-chloronicotinate (20 g, 0.1 mol) and tert-butyl piperazine-1-carboxylate (18.6 g, 0.1 mol) in THF (200 mL) was heated to reflux. Then triethylamine (12 g, 0.12 mol) was added. The reaction mixture was heated overnight. Solvent was removed under vacuo. To the residue was added water (200 mL), extracted with ethyl acetate (3×200 mL). The organic layer was dried over Na₂SO₄, concentrated to give the crude product. It was purified by column chromatography by silica gel column eluting with 1:15 ratio of ethyl acetate in petroleum ether to give the title compound (10 g, 29%) as white solid. LC-MS m/z 350.2 (M+H)⁺, 1.80 min (ret time)

Intermediate 50:1-Methylethyl 2-(1-piperazinyl)-3-pyridinecarboxylate

### (Preparation 1)

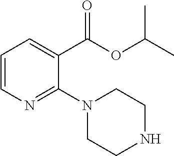 [SMILES: CC(C)OC(=O)c1cccnc1N1CCNCC1]

To the solution of 1,1-dimethylethyl 4-(3-{[(1-methylethyl)oxy]carbonyl}-2-pyridinyl)-1-piperazinecarboxylate (10 g, 29 mmol) in CH₂Cl₂ (150 mL) was added 2,2,2-trifluoroacetic acid (20 mL). The reaction mixture was stirred at room temperature overnight. Solvent was removed under vacuo. To the residue was added saturated Na₂CO₃ (200 mL) and extracted with ethyl acetate (3×200 mL). The organic layer was dried over Na₂SO₄ and concentrated to give the title compound (7.13 g, 100%) as yellow oil. LC-MS m/z 250.0 (M+H)⁺, 0.89 min (ret time)

### (Preparation 2)

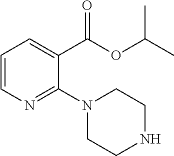 [SMILES: CC(C)OC(=O)c1cccnc1N1CCNCC1]

1-Methylethyl 2-(1-piperazinyl)-3-pyridinecarboxylate hydrochloride (800 mg) was suspended in EtOAc (75 mL) and shaken with 1N aq NaOH (25 mL) and the solid dissolved. The EtOAc was washed again with 1 N aq NaOH (25 mL) and then with water (25 mL) and then satd aq NaCl (25 mL), dried (Na₂SO₄) and concentrated to afford 1-methylethyl 2-(1-piperazinyl)-3-pyridinecarboxylate free base as a clear oil.

### Intermediate 51: 1-Methylethyl 2-[4-({4-[bis(ethyloxy)methyl]phenyl}methyl)-1-piperazinyl]-3-pyridinecarboxylate

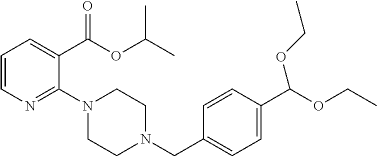 [SMILES: CCOC(OCC)c1ccc(CN2CCN(c3ncccc3C(=O)OC(C)C)CC2)cc1]

The solution of 1-methylethyl 2-(1-piperazinyl)-3-pyridinecarboxylate (1.5 g, 6 mmol) and 4-(diethoxymethyl)benzaldehyde (1.248 g, 6 mmol) in THF (8 mL) was stirred at room temperature for 10 min, NaBH(AcO)₃ (3.831 g, 18 mmol) was added. The reaction mixture was stirred overnight. To the reaction mixture was added water and extracted with ethyl acetate. The organic layer was washed with brine, dried over Na₂SO₄ and concentrated to give the crude product. It was purified by silica gel column eluting with 1:30 to 1:2 ratio of ethyl acetate in petroleum ether to give the title compound (1.3 g, 49%) as yellow oil. LC-MS m/z 442.3 (M+H)⁺, 1.93 min (ret time)

### Intermediate 52: 1-Methylethyl 2-{4-[(4-formylphenyl)methyl]-1-piperazinyl}-3-pyridine carboxylate

### (Preparation 1)

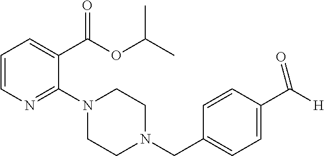 [SMILES: CC(C)OC(=O)c1cccnc1N1CCN(Cc2ccc(C=O)cc2)CC1]

The solution of 1-methylethyl 2-[4-({4-[bis(ethyloxy)methyl]phenyl}methyl)-1-piperazinyl]-3-pyridinecarboxylate (800 mg, 1.81 mmol), HCl (5 mL, 1 mol/L) in acetone (10 mL) was heated at 80° C. for 1 h. The reaction mixture was cooled to room temperature; saturated NaHCO₃ was added and extracted with ethyl acetate. The organic layer was dried over Na₂SO₄ and concentrated to give the title compound (641 mg, 96%) as yellow oil. LC-MS m/z 368.1 (M+H)⁺, 1.73 min (ret time)

### (Preparation 2)

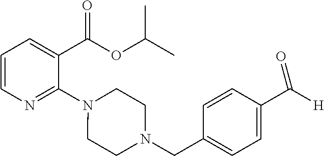 [SMILES: CC(C)OC(=O)c1cccnc1N1CCN(Cc2ccc(C=O)cc2)CC1]

1-methylethyl 2-(1-piperazinyl)-3-pyridinecarboxylate (4.99 g, 20 mmol), Acetonitrile (8.00 ml), 4-(bromomethyl)benzaldehyde (3.98 g, 20 mmol), and K₂CO₃ (3.32 g, 24 mmol) were combined and heated to 50° C. for 5 h. The reaction was diluted with EtOAc (400 mL) and washed with 1 M aq NaOH (100 mL). The aq was back extracted with EtOAc (75 mL) and the combined extracts were washed with water (75 mL), satd aq NaCl (75 mL) and dried (Na₂SO₄), filtered and concentrated to a brown oil. The crude product was purified on a silica cartridge (120 g) eluting at 85 mL/min with a gradient running from 10% EtOAc/hexanes to 40% EtOAc/hexanes over 35 min. The desired fractions were pooled and concentrated to afford the title compound as a clear yellow oil which solidified on standing at 23° C. (3.51 g, 9.55 mmol, 47.8% yield). Lcms rt 0.78[M+H]=368.3.

### (Preparation 3)

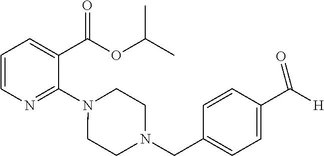 [SMILES: CC(C)OC(=O)c1cccnc1N1CCN(Cc2ccc(C=O)cc2)CC1]

To a solution of 1-methylethyl 2-(1-piperazinyl)-3-pyridinecarboxylate (3.59 g, 14.40 mmol) in Acetone (20 mL) was added 4-(bromomethyl)benzaldehyde (2.58 g, 12.96 mmol) and K2CO3 (5.97 g, 43.2 mmol) and heated to 50° C. for 3 hours. The reaction was filtered and concentrated. The residue was purified by silica column eluting with 0-50% EtOAc/DCM to obtain the title compound (3.53 g, 66%). LC-MS m/z=368 (M+H), 1.10 minutes (retention time).

### Intermediate 53: 3-(Hydroxymethyl)benzaldehyde

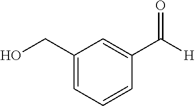

To a solution of isophthalaldehyde (10 g, 74.56 mmol) in THF (50 mL) and EtOH (100 mL) at 0° C. was added NaBH₄ (1 g, 26.43 mmol) portion wise, the resulting solution was stirred at 0° C. for 10 min. 10% HCl (aq. 50 mL) was added, the mixture was extracted with EtOAc (3×150 mL). The combined organic layer was washed with brine (2×50 mL), dried over MgSO₄, concentrated to give the title product (10 g, 99%) as yellow oil. LC-MS m/z 137.1 (M+H)⁺, 1.01 min (ret time).

### Intermediate 54: 1-Methylethyl 2-[(3R)-3-({[(1,1-dimethylethyl)oxy]carbonyl}amino)-1-pyrrolidinyl]-3-pyridinecarboxylate

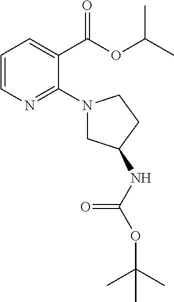 [SMILES: CC(C)OC(=O)c1cccnc1N1CC[C@@H](NC(=O)OC(C)(C)C)C1]

The solution of isopropyl 2-chloronicotinate (4 g, 20 mmol) and (R)-tert-butyl pyrrolidin-3-ylcarbamate (4.5 g, 24 mmol) in THF (200 mL) was heated to reflux. Et₃N (2.4 g, 24 mmol) was added and it was stirred overnight. Solvent was removed. To the residue was added water (200 mL), extracted with ethyl acetate (3×200 mL). The organic layer was dried over Na₂SO₄ and concentrated to give the title compound (6 g, 86%) as yellow oil. LC-MS m/z 350.1 (M+H)⁺, 1.26 min (ret time)

### Intermediate 55: 1-Methylethyl 2-{(3R)-3-[{[(1,1-dimethylethyl)oxy]carbonyl}(ethyl)amino]-1-pyrrolidinyl}-3-pyridinecarboxylate

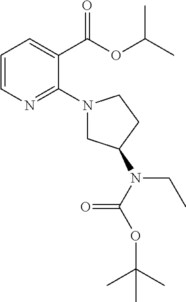 [SMILES: CCN(C(=O)OC(C)(C)C)[C@@H]1CCN(c2ncccc2C(=O)OC(C)C)C1]

To the solution of 1-methylethyl 2-[(3R)-3-({[(1,1-dimethylethyl)oxy]carbonyl}amino)-1-pyrrolidinyl]-3-pyridinecarboxylate (6 g, 17 mmol) in DMF (20 mL) was added NaH (60%, 1.65 g, 41 mmol), stirred at room temperature for 10 min. To the reaction mixture was added iodoethane (5.36 g, 34 mmol), stirred at 40° C. for 2 h. To the reaction mixture was added water (200 mL), extracted with ethyl acetate (3×200 mL). The organic layer was washed with brine (4×200 mL), dried over Na₂SO₄ and concentrated to give the title compound (6.4 g, 98.7%) as yellow solid. LC-MS m/z 378.1 (M+H)⁺, 1.43 min (ret time)

### Intermediate 56: 1-Methylethyl 2-{4-[(3-nitrophenyl)methyl]-1-piperazinyl}-3-pyridine carboxylate

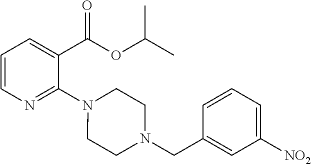 [SMILES: CC(C)OC(=O)c1cccnc1N1CCN(Cc2cccc([N+](=O)[O-])c2)CC1]

A mixture of 1-methylethyl 2-(1-piperazinyl)-3-pyridinecarboxylate (800 mg, 3.2 mmol) and 3-nitrobenzaldehyde (0.48 g, 3.2 mmol) in THF (100 mL) was stirred at RT for 10 min. NaBH(CH₃CO₂)₃ (1.2 g, 5.6 mmol) was added. The reaction mixture was stirred at RT overnight. Water (10 mL) was added. The water layer was extracted with ethyl acetate. The organic layer was washed with brine, dried and concentrated to give desired product (1.25 g, crude) as yellow oil. LC-MS m/z 385.0 (M+H)⁺, 1.84 min (ret time)

### Intermediate 57: 1-Methylethyl 2-{4-[3-aminophenyl)methyl]-1-piperazinyl}-3-pyridinecarboxylate

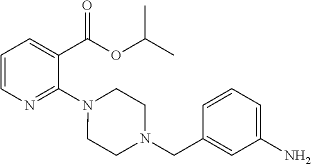 [SMILES: CC(C)OC(=O)c1cccnc1N1CCN(Cc2cccc(N)c2)CC1]

To a solution of 1-methylethyl 2-{4-[(3-nitrophenyl)methyl]-1-piperazinyl}-3-pyridinecarboxylate (1.25 g, 3.2 mmol) in methanol (50 mL) at RT was added a solution of NaHS (1.8 g, 32 mmol) in water (20 mL). The mixture was heated at 75° C. overnight. The reaction mixture was diluted with water, extracted with ethyl acetate. The organic layer was washed by water and brine, dried and concentrated to give the desired product (1.2 g, 100%) as yellow solid. LC-MS m/z 355.1 (M+H)⁺, 1.03 min (ret time)

### Intermediate 58: 1-Methylethyl 2-[4-({3-[(phenylcarbonyl)amino]phenyl}methyl)-1-piperazinyl]-3-pyridinecarboxylate

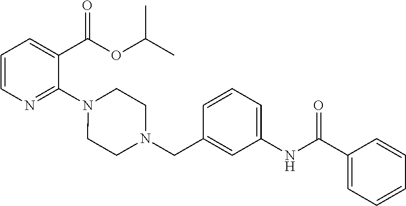 [SMILES: CC(C)OC(=O)c1cccnc1N1CCN(Cc2cccc(NC(=O)c3ccccc3)c2)CC1]

To a solution of 1-methylethyl 2-{4-[(3-aminophenyl)methyl]-1-piperazinyl}-3-pyridinecarboxylate (590 mg, 1.66 mmol) and benzoic acid (203 mg, 1.66 mmol) in THF (100 mL) were added EDCI (317 mg, 1.66 mmol) and HOBt (224 mg, 1.66 mmol). The reaction mixture was stirred at room temperature overnight. The reaction mixture was diluted with ethyl acetate. The organic layer was washed with water and brine, dried and concentrated to give the desired product (700 mg, 92%) as yellow solid. LC-MS m/z 459.1 (M+H)⁺, 1.77 min (ret time)

### Intermediate 59: 1-Methylethyl2-{4-[4-nitrophenyl)methyl]-1-piperazinyl}-3-pyridine carboxylate

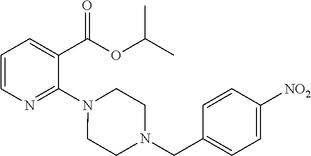 [SMILES: CC(C)OC(=O)c1cccnc1N1CCN(Cc2ccc([N+](=O)[O-])cc2)CC1]

A mixture of 1-methylethyl 2-(1-piperazinyl)-3-pyridinecarboxylate (800 mg, 3.2 mmol) and 4-nitrobenzaldehyde (0.48 g, 3.2 mmol) in THF (100 mL) was stirred at RT for 10 min. NaBH(CH₃CO₂)₃ (1.2 g, 5.6 mmol) was added. The reaction mixture was stirred at RT overnight. Water (10 mL) was added. The water layer was extracted with ethyl acetate. The organic layer was washed with brine, dried and concentrated to give the crude desired product (1.17 g) as yellow oil. LC-MS m/z 385.1 (M+H)⁺, 1.13 min (ret time)

### Intermediate 60:1-Methylethyl 2-{4-[(4-aminophenyl)methyl]-1-piperazinyl}-3-pyridine carboxylate

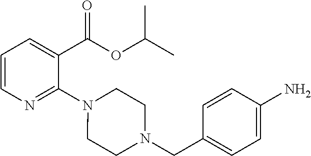 [SMILES: CC(C)OC(=O)c1cccnc1N1CCN(Cc2ccc(N)cc2)CC1]

The procedure was similar with 1-Methylethyl 2-{4-[(3-aminophenyl)methyl]-1-piperazinyl}-3-pyridinecarboxylate

1-Methylethyl 2-{4-[(4-nitrophenyl)methyl]-1-piperazinyl}-3-pyridinecarboxylate (1.17 g, 3 mmol) and NaHS (1.7 g, 30 mmol) were reacted to give the title compound (1.2 g, 100%) as yellow solid. LC-MS m/z 365.1 (M+H)⁺, 1.61 min (ret time)

### Intermediate 61: 1-Methylethyl 2-[4-({4-[(phenylcarbonyl)amino]phenyl}methyl)-1-piperazinyl]-3-pyridinecarboxylate

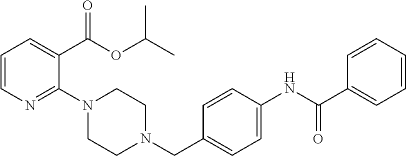 [SMILES: CC(C)OC(=O)c1cccnc1N1CCN(Cc2ccc(NC(=O)c3ccccc3)cc2)CC1]

The procedure was similar with 1-Methylethyl 2-[4-({3-[(phenylcarbonyl)amino]phenyl}methyl)-1-piperazinyl]-3-pyridinecarboxylate 1-Methylethyl 2-{4-[(4-aminophenyl)methyl]-1-piperazinyl}-3-pyridinecarboxylate (730 mg, 2.06 mmol), benzoic acid (251.5 mg, 2.06 mmol), EDCI (393 mg, 2.06 mmol) and

HOBt (278 mg, 2.06 mmol) were reacted to give the title compound (800 mg, 85%) as yellow solid. LC-MS m/z 459.1 (WH)⁺, 1.76 min (ret time)

### Intermediate 62: 3-{[4-(3-{[(1-Methylethyl)oxy]carbonyl}-2-pyridinyl)-1-piperazinyl]methyl}benzoic acid

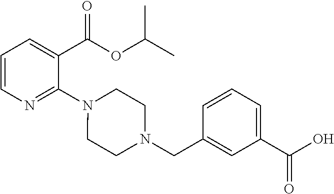 [SMILES: CC(C)OC(=O)c1cccnc1N1CCN(Cc2cccc(C(=O)O)c2)CC1]

The procedure was similar with 1-Methylethyl 2-{4-[(4-nitrophenyl)methyl]-1-piperazinyl}-3-pyridinecarboxylate 1-Methylethyl 2-(1-piperazinyl)-3-pyridinecarboxylate (500 mg, 2 mmol), 3-formylbenzoic acid (301 mg, 2 mmol) and NaBH(CH₃CO₂)₃ (550 mg, 2.6 mmol) were reacted to give the title compound (700 mg, 91%) as yellow solid. LC-MS m/z 384.0 (M+H)⁺, 1.30 min (ret time)

### Intermediate 63: 4-{[4-(3-{[(1-Methylethyl)oxy]carbonyl}-2-pyridinyl)-1-piperazinyl]methyl}benzoic acid

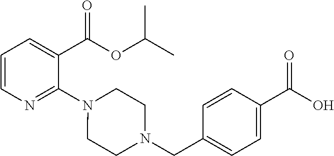 [SMILES: CC(C)OC(=O)c1cccnc1N1CCN(Cc2ccc(C(=O)O)cc2)CC1]

The procedure was similar with 1-Methylethyl 2-{4-[(4-nitrophenyl)methyl]-1-piperazinyl}-3-pyridinecarboxylate 1-Methylethyl 2-(1-piperazinyl)-3-pyridinecarboxylate (500 mg, 2 mmol), 4-formylbenzoic acid (301 mg, 2 mmol) and NaBH(CH₃CO₂)₃ (550 mg, 2.6 mmol) were reacted to give the title compound (700 mg, 91%) as yellow solid. LC-MS m/z 384.1 (M+H)⁺, 1.30 min (ret time)

### Intermediate 64: 3-{[(2-Chloro-6-fluorophenyl)methyl]oxy}benzaldehyde

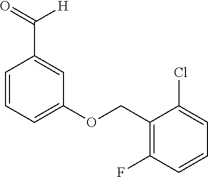 [SMILES: O=Cc1cccc(OCc2c(F)cccc2Cl)c1]

To a solution of 3-hydroxybenzaldehyde (244.24 mg, 2 mmol) and 2-(bromomethyl)-1-chloro-3-fluorobenzene (446.94 mg, 2 mmol) in acetone (50 mL), was added K₂CO₃ (500 mg, 3.6 mmol). The reaction mixture was refluxed for 3 h, filtered and evaporated to obtain the desired product (560 mg, 100%) as colorless oil. LC-MS m/z 264.9 (M+H)⁺, 1.59 min (ret time)

### Intermediate 65: 1-Methylethyl2-{[((2S)-{[(1,1-dimethylethyl)oxy]carbonyl}-2-pyrrolidinyl)methyl]oxy}-3-pyridinecarboxylate

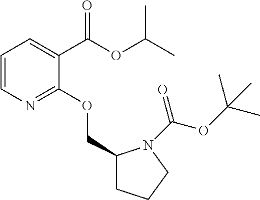 [SMILES: CC(C)OC(=O)c1cccnc1OC[C@@H]1CCCN1C(=O)OC(C)(C)C]

To a solution of Isopropyl 2-hydroxynicotinate (8.25 g, 45.5 mmol) in THF (200 ml) under argon at −5° C., was added triphenylphosphine (17.91 g, 68.3 mmol), (S)-tert-butyl 2-(hydroxymethyl)pyrrolidine-1-carboxylate (13.75 g, 68.3 mmol). DIAD (13.28 mL, 68.3 mmol) in THF (50 mL) was then added to the reaction mixture dropwise. The reaction mixture was warmed to room temperature and stirred overnight. Solvent was removed under reduced pressure. The residue was purified by silica gel column chromatography eluting with 100% petroleum ether to 25% ethyl acetate in petroleum ether to obtain the desired compound (9.36 g, 56.4%) as colorless oil. LC-MS m/z 365.0 (M+H)⁺, 1.72 min (ret time); ¹H NMR (400 MHz, DMSO-d₆) δ 1.28-1.30 (d, 6H) 1.40 (s, 9H) 1.62-1.66 (m, 2H) 1.84-1.90 (m, 2H) 3.37-3.50 (m, 2H) 3.50-3.52 (m, 2H) 5.10-5.13 (m, 1H) 5.37-5.39 (m, 1H) 7.06-7.09 (m, 1H) 8.07-8.09 (m, 1H) 8.32-8.34 (m, 1H).

### Intermediate 66: 1-Methylethyl 2-{[((2R)-1-{[(1,1-dimethylethyl)oxy]carbonyl}-2-pyrrolidinyl)methyl]oxy}-3-pyridinecarboxylate

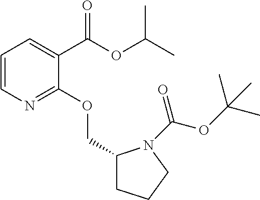 [SMILES: CC(C)OC(=O)c1cccnc1OC[C@H]1CCCN1C(=O)OC(C)(C)C]

To a solution of Isopropyl 2-hydroxynicotinate (8.25 g, 45.5 mmol) in THF (200 mL) under argon at −5° C., was added triphenylphosphine (17.91 g, 68.3 mmol), 1,1-dimethylethyl (2R)-2-(hydroxymethyl)-1-pyrrolidinecarboxylate (13.75 g, 68.3 mmol). DIAD (13.28 mL, 68.3 mmol) in THF (50 mL) was then added to the reaction mixture dropwise. The reaction mixture was warmed to room temperature and stirred overnight. Solvent was removed under reduced pressure. The residue was purified by silica gel column chromatography eluting with 100% petroleum ether to 25% ethyl acetate in petroleum ether to obtain the desired compound (8.58 g, 34.6%) as red oil. LC-MS m/z 365.2 (M+H)⁺, 1.90 min (ret time)

### Intermediate 67:Methyl 2-chloro-3-pyridinecarboxylate

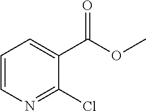

To a suspension of 2-chloronicotinic acid (10 g, 0.06 mol) in dichloromethane (30 mL) was added oxalyl chloride (19.33 g, 0.15 mol) and DMF (0.2 mL) dropwise. The mixture was stirred at room temperature for 45 min. Solvent was removed in vacuum to obtain 2-chloronicotinoyl chloride as yellow solid. 2-Chloronicotinoyl chloride was then added to a solution of Et₃N (5 mL) in methanol (30 mL) in portions. The reaction mixture was allowed to stir at room temperature for 1 h. Water was added and extracted with EtOAc. The combined organic layer was washed with saturated Na₂CO₃, dried over Na₂SO₄ and concentrated to obtain the title compound (10 g, 92%) as yellow oil. LC-MS m/z 172 (M+H)⁺, 1.36 min (ret time)

### Intermediate 68:(2-Chloro-3-pyridinyl)methanol

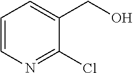

To a solution of methyl 2-chloronicotinate (2.5 g, 15 mmol) in THF (30 mL) was added LiAlH₄(1.109 mg, 30 mmol) at 0° C. in a water/ice bath. The reaction was allowed to warm to room temperature and stirred for 30 min. Then to the reaction mixture was added Na₂SO₄.10H₂O (4.7 g). Solid was removed though filtration. The filtrate was concentrated to obtain the title compound (2.08 g, 100%) as yellow oil. LC-MS m/z 144 (M+H)⁺, 0.87 min (ret time)

### Intermediate 69: 2-Oxo-1,2-dihydro-3-pyridinecarbonyl chloride

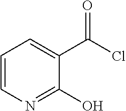

To a suspension of 2-hydroxynicotinic acid (50 g, 0.36 mol) and oxalyl chloride (54.7 g, 0.43 mol) in dichloromethane (250 mL) was added DMF (1 mL) dropwise. The mixture was stirred at room temperature for 30 min. Solvent was removed to obtain the title compound (56.6 g, 100%) as yellow solid.

### Intermediate 70:1-Methylethyl 2-oxo-1,2-dihydro-3-pyridinecarboxylate

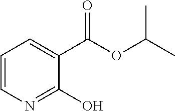

To a solution of Et₃N (30 mL) in propan-2-ol (180 mL) was added 2-hydroxynicotinoyl chloride (56.7 g) in portions. The reaction mixture was stirred at room temperature for 2 h. Solvent was removed. Water was added to the residue and extracted with EtOAc. The combined organic layer was washed with saturated NaHCO₃, dried over Na₂SO₄ and concentrated to obtain the title compound (65 g, 100%) as white solid. LC-MS m/z 182(M+H)⁺, 1.02 min (ret time)

### Intermediate 71: 1,1-Dimethylethyl ethyl{(3R)-1-[3-(hydroxymethyl)-2-pyridinyl]-3-pyrrolidinyl}carbamate

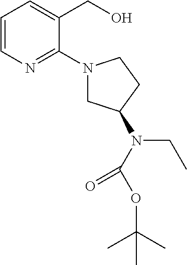 [SMILES: CCN(C(=O)OC(C)(C)C)[C@@H]1CCN(c2ncccc2CO)C1]

To the solution of 1-methylethyl 2-{(3R)-3-[{[(1,1-dimethylethyl)oxy]carbonyl}(ethyl)amino]-1-pyrrolidinyl}-3-pyridinecarboxylate (2 g, 5 mmol) in THF (10 mL) was added LiAlH₄ (210 mg, 5 mmol), stirred at room temperature for 30 min. To the reaction mixture was added Na₂SO₄.10H₂O (2 g), stirred for another 30 min. The reaction mixture was filtrated. The filtrate was concentrated to give the title compound (1.7 g, 100%) as yellow solid. LC-MS m/z 322.1 (M+H)⁺, 0.99 min (ret time)

### Intermediate 72:(2-{(3R)-3-[{[(1,1-Dimethylethyl)oxy]carbonyl}(ethyl)amino]-1-pyrrolidinyl}-3-pyridinyl)methyl benzoate

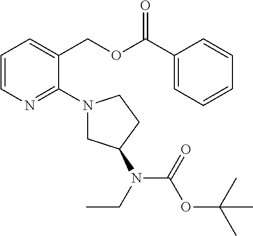 [SMILES: CCN(C(=O)OC(C)(C)C)[C@@H]1CCN(c2ncccc2COC(=O)c2ccccc2)C1]

To the solution of benzoic acid (732 mg, 6 mmol) in CH₂Cl₂ (3 mL) was added oxalyl dichloride (914 mg, 7.2 mmol). It was stirred at room temperature for 30 min. Solvent was removed. The residue was dissolved in CH₂Cl₂ (3 mL), 1,1-dimethylethyl ethyl{(3R)-1-[3-(hydroxymethyl)-2-pyridinyl]-3-pyrrolidinyl}carbamate (963 mg, 3 mmol) and Et₃N (0.9 mL) were added. It was stirred at room temperature for another 2 h. To the reaction mixture was added CH₂Cl₂ (30 mL), filtrated. The filtrate was concentrated to give the crude product. It was purified by silica gel column eluting with 1:10 ratio of ethyl acetate in petroleum ether to give the title compound (360 mg, 28%) as yellow oil. LC-MS m/z 426.2 (M+H)⁺, 1.45 min (ret time)

### Intermediate 73: {2-[(3R)-3-(Ethylamino)-1-pyrrolidinyl]-3-pyridinyl}methyl benzoate

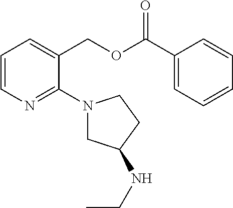 [SMILES: CCN[C@@H]1CCN(c2ncccc2COC(=O)c2ccccc2)C1]

To the solution of (2-{(3R)-3-[{[(1,1-dimethylethyl)oxy]carbonyl}(ethyl)amino]-1-pyrrolidinyl}-3-pyridinyl)methyl benzoate (910 mg, 2.14 mmol) in CH₂Cl₂ (30 mL) was added 2,2,2-trifluoroacetic acid (2 mL). It was stirred at room temperature for 2 h. Solvent was removed. To the residue was added saturated Na₂CO₃, adjusted Ph≧7, extracted with ethyl acetate (3×30 mL). The organic layer was dried over Na₂SO₄ and concentrated to give the title compound (696 mg, 100%) as yellow oil. LC-MS m/z 326.1 (M+H)⁺, 0.93 min (ret time)

### Intermediate 74: (2-{(3R)-3-[{[(1,1-Dimethylethyl)oxy]carbonyl}(ethyl)amino]-1-pyrrolidinv1}-3-pyridinyl)methyl 3,3-dimethylbutanoate

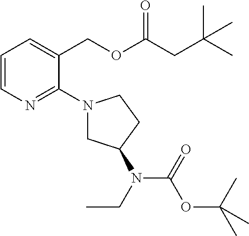 [SMILES: CCN(C(=O)OC(C)(C)C)[C@@H]1CCN(c2ncccc2COC(=O)CC(C)(C)C)C1]

To a solution of 1,1-dimethylethyl ethyl{(3R)-1-[3-(hydroxymethyl)-2-pyridinyl]-3-pyrrolidinyl}carbamate (500 mg, 1.55 mmol) and 3,3-dimethylbutanoic acid (180.7 mg, 1.55 mmol) in DCM(40 mL) was added EDCI (592.1 mg, 3.1 mmol) and HOBt (418.5 mg, 3.1 mmol). The reaction mixture was stirred at room temperature overnight. Water (40 mL) was added, extracted with DCM. The organic layer was washed with water and brine, dried and concentrated. The residue was purified by silica gel column chromatography eluting with 8:1 ratio of petroleum ether in ethyl acetate to give the desired product (342.8 mg, 52.4%) as pale yellow oil. LC-MS m/z 420.2 (M+H)⁺, 1.29 min (ret time)

### Intermediate 75: (2-{(3R)-3-[{[(1,1-Dimethylethyl)oxy]carbonyl}(ethyl)amino]-1-pyrrolidinyl}-3-pyridinyl)methyl 3,3-dimethylbutanoate

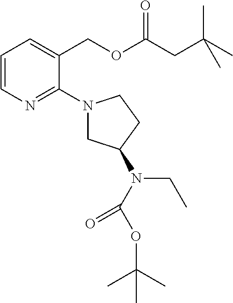 [SMILES: CCN(C(=O)OC(C)(C)C)[C@@H]1CCN(c2ncccc2COC(=O)CC(C)(C)C)C1]

To the solution of 3,3-dimethyl butanoic acid (464 mg, 4 mmol) in CH₂Cl₂ (2 mL) was added oxalyldichloride (609 mg, 4.8 mmol). It was stirred at room temperature for 30 min. Solvent was removed. The residue was dissolved in CH₂Cl₂ (2 mL), 1,1-dimethylethyl ethyl{(3R)-1-[3-(hydroxymethyl)-2-pyridinyl]-3-pyrrolidinyl}carbamate (642 mg, 2 mmol) and Et₃N (0.6 mL) were added. It was stirred at room temperature for another 2 h. To the reaction mixture was added CH₂Cl₂ (50 mL), filtrated. The filtrate was concentrated to give the crude product. It was purified by silica gel column eluting with 1:8 ratio of ethyl acetate in petroleum ether to give the title compound (560 mg, 67%) as colorless oil. LC-MS m/z 420.3 (M+H)⁺, 1.50 min (ret time)

### Intermediate 76: {2-[(3R)-3-(Ethylamino)-1-pyrrolidinyl}-3-pyridinyl]methyl 3,3-dimethylbutanoate

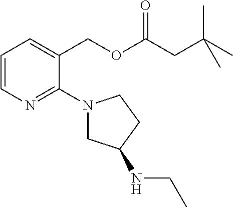 [SMILES: CCN[C@@H]1CCN(c2ncccc2COC(=O)CC(C)(C)C)C1]

To the solution of (2-{(3R)-3-[{[(1,1-dimethylethyl)oxy]carbonyl}(ethyl)amino]-1-pyrrolidinyl}-3-pyridinyl)methyl 3,3-dimethylbutanoate (560 mg, 1.34 mmol) in CH₂Cl₂ (20 mL) was added 2,2,2-trifluoroacetic acid (1 mL). It was stirred at room temperature for 2 h. Solvent was removed. To the residue was added saturated Na₂CO₃, adjusted Ph 7, extracted with ethyl acetate (3×20 mL). The organic layer was dried over Na₂SO₄ and concentrated to give the title compound (426 mg, 100%) as yellow oil. LC-MS m/z 320.2 (M+H)⁺, 1.16 min (ret time)

### Intermediate 77: 1-Methylethyl 2-{[(2S)-2-pyrrolidinylmethyl]oxy}-3-pyridinecarboxylate

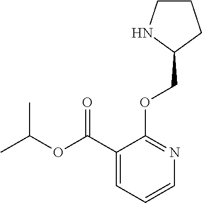 [SMILES: CC(C)OC(=O)c1cccnc1OC[C@@H]1CCCN1]

To a solution of (S)-isopropyl 2-((1-(tert-butoxycarbonyl)pyrrolidin-2-yl) (9.6 g, 26.3 mmol) in DCM (150 mL) under ice-cold bath was added TFA (100 mL). The reaction mixture was stirred at room temperature for 2 h. Solvent was removed under reduced pressure. Saturated NaHCO₃ solution was added to the residue and extracted with ethyl acetate. The organic layer was dried, concentrated to obtain the desired compound (5.5 g, 79.1%) as pale yellow solid. LC-MS m/z 265.0 (M+H)⁺, 1.47 min (ret time); ¹H NMR (400 MHz, DMSO-d₆) δ 1.31 (d, J=6.4 Hz, 6H), 1.84-2.13 (m, 4 H), 3.23 (m, 2H), 3.96 (m, 1H), 4.43 (m, 1H), 4.54 (m, 1H), 5.12 (m, 1H), 7.16 (m, 1 H), 8.15 (m, 1H), 8.38 (m, 1H).

### Intermediate 78: 1-Methylethyl 2-{[(2R)-2-pyrrolidinylmethyl]oxy}-3-pyridinecarboxylate

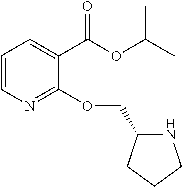 [SMILES: CC(C)OC(=O)c1cccnc1OC[C@H]1CCCN1]

To a solution of (S)-isopropyl 2-((1-(tert-butoxycarbonyl)pyrrolidin-2-yl) (8.58 g, 263.5 mmol) in DCM (150 mL) under ice-cold bath was added TFA (100 mL). The reaction mixture was stirred at room temperature for 2 h. Solvent was removed under reduced pressure. Saturated NaHCO₃ solution was added to the residue and extracted with ethyl acetate. The organic layer was dried, concentrated to obtain the desired compound (6.0 g, 96%) as brown oil. LC-MS m/z 265.1 (M+H)⁺, 1.46 min (ret time); ¹H NMR (400 MHz, DMSO-d₆) δ 1.31 (d, J=5.2 Hz, 6H), 1.81-1.86 (m, 2H), 1.98-2.10 (m, 2H), 3.16-3.19 (m, 2H), 4.02-4.03 (m, 1H), 4.38-4.42 (m, 1H), 4.50-4.53 (m, 1H), 5.10-5.12 (m, 1H), 7.14-7.17 (m, 1H), 8.14-8.16 (m, 1H), 8.36-8.38 (m, 1H).

### Intermediate 79 1-Methylethyl 2-{4-[(3-hydroxyphenyl)methyl]-1-piperazinyl}-3-pyridinecarboxylate

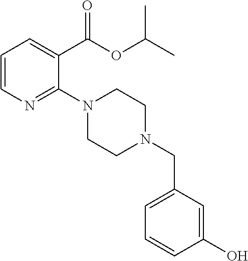 [SMILES: CC(C)OC(=O)c1cccnc1N1CCN(Cc2cccc(O)c2)CC1]

1-Methylethyl 2-(1-piperazinyl)-3-pyridinecarboxylate (386 mg, 1.55 mmol) free base, 3-hydroxybenzaldehyde (189 mg, 1.55 mmol), and tetrahydrofuran (THF) (7 mL) were combined in a 20 mL vial and stirred together for 5 min and then sodium triacetoxyborohydride (984 mg, 4.68 mmol) was added. The mixture was stirred 6 h at 23° C. and then was diluted with EtOAc (75 mL) and washed with 1 N aq NaOH (25 mL), H₂O (25 mL) and satd aq NaCl (25 mL), dried (Na₂SO₄) and concentrated to afford the title compound as a yellow oil, 549 mg (87%). Lcms rt=0.66[M+H]=356.2. Purity by LCMS 87%.

### Intermediate 80:1-Methylethyl 2-{(3R)-3-(ethylamino)-1-pyrrolidinyl}-3-pyridine carboxylate dihydrochloride

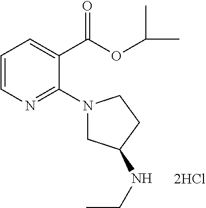 [SMILES: CCN[C@@H]1CCN(c2ncccc2C(=O)OC(C)C)C1]

To a solution of 1-methylethyl 2-{(3R)-3-[{[(1,1-dimethylethyl)oxy]carbonyl}(ethyl)amino]-1-pyrrolidinyl}-3-pyridinecarboxylate (17 g, 45.0 mmol) in methanol (100 mL), 4.0 M HCl in dioxane (56.3 mL, 225 mmol) was added. The reaction mixture was stirred at 25° C. for 1.5 h. Then all solvent was evaporated and dried under vacuum pump for 16 h to give the title compound 17 g. It was kept as an intermediate without further purification. LC-MS m/z 277.9 (M+H)⁺, 0.67 min (ret time)

### Intermediate 81: [(2-chloro-6-fluorophenyl)methyl]ethylamine Preparation 1

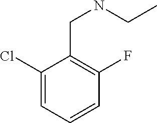

2-chloro-6-fluorobenzaldehyde (2.378 g, 15.00 mmol) was dissolved in 1,2-Dichloroethane (DCE) (29.1 ml) and cooled to 4° C. on an ice bath. 2M ethylamine in THF (30.0 ml, 60.0 mmol) was added and the solution was stirred at 4° C. for 15 min and then acetic acid (0.859 ml, 15.00 mmol) and sodium triacetoxyborohydride (3.18 g, 15.00 mmol) was added. The resulting mixture was stirred for 30 min and then was warmed to 23° C. and stirred for 16 h. The reaction was diluted with EtOAc (200 mL) and 1M aq NaOH (50 mL), the phases were separated and the aqueous was extracted again with EtOAc (50 mL) and the combined EtOAc was washed with 50 mL each of water, and satd aq NaCl, dried (Na₂SO₄) and concentrated to afford 2.77 of an orange residue. The crude product was purified on a silica cartridge (80 g) with a Combiflash Companion, eluting at 60 mL/min with a gradient running from dichloromethane to ethyl acetate over 30 min. The desired fractions were pooled and concentrated to afford [(2-chloro-6-fluorophenyl)methyl]ethylamine (586 mg, 3.12 mmol, 20.82% yield) as a clear light yellow oil. ¹H NMR (400 MHz, DMSO-d₆) δ 7.32-7.51 (m, 2H), 7.17-7.33 (m, 1H), 3.79-3.94 (m, 2H), 2.68 (m, 2), 0.99-1.14 (m, 3H).

### Preparation 2: Intermediate: [(2-chloro-6

fluorophenyl)methyl]ethylamine

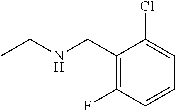

To a solution of 2-chloro-6-fluorobenzaldehyde (3.00 g, 18.92 mmol) in Methanol (3 mL) was added 2M Ethylamine in THF (47.3 mL, 95 mmol) followed by Acetic Acid (1.083 mL, 18.92 mmol). The mixture was stirred at room temperature for 45 minutes. Sodium cyanoborohydride (4.16 g, 66.2 mmol) was then added and the reaction was stirred at room temperature for 18 hours. Afterwards, the reaction was quenched with 5 ml water, and the solvent was concentrated. The residue was dissolved in EtOAc and washed with water (2×). The aqueous layer was backextracted with EtOAc, washed combined organics with water, brine, dried MgSO4, and concentrated the solvent. The residue was purified by silica column eluting with 10-50% of a (10% bottle of 2N NH3/MeOH/DCM)/with DCM to obtain the title compound (1.73 g, 48%). LC-MS m/z=188 (M+H), 0.46 minutes (retention time).

### Intermediate 82 2-bromo-5-({[(1,1-dimethylethyl)(dimethyl)silyl]oxy}methyl)pyridine

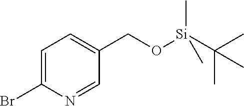 [SMILES: CC(C)(C)[Si](C)(C)OCc1ccc(Br)nc1]

(6-Bromo-3-pyridinyl)methanol (5 g, 26.6 mmol), triethylamine (5.56 ml, 39.9 mmol), in N,N-dimethylformamide (DMF) (9.21 ml) and TBDMSCI (4.01 g, 26.6 mmol) were stirred at 23 C for 16 h and then more of TBDMSCI (8.02 g, 53.2 mmol), and more of triethylamine (7.41 ml, 53.2 mmol) were added and stirred for 4 h. The solvent was removed in vacuo and the residue was dissolved in EtOAc, washed three times with satd aq NaCl, dried (MgSO₄), concentrated under a stream of nitrogen at 50° C., then high vacuum. The crude product was purified by chromatography on a silica cartridge (80 g) eluting with a gradient from dichloromethane to 5% EtOAc in dichloromethane to give 5.6915 g (70.8%) of the title compound. (LC-MS m/z 302/304 (M+H)⁺ 1.42 (ret time)

### Intermediate 83: 5-({[(1,1-dimethylethyl)(dimethyl)silyl]oxy}methyl)-2-pyridine carbaldehyde

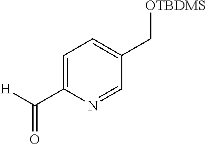 [SMILES: CC(C)(C)[Si](C)(C)OCc1ccc(C=O)nc1]

To 2-bromo-5-({[(1,1-dimethylethyl)(dimethyl)silyl]oxy}methyl)pyridine (4.5407 g, 15.02 mmol) in dry THF (200 ml) under an argon atmosphere was slowly added nBuLi 2.5M in hexane (6.61 ml, 16.52 mmol) at −78° C. After 40 min at −78° C. dry DMF (1.279 ml, 16.52 mmol) was added. the mixture was stirred one hour and then warmed to 23° C. The solvent was removed in vacuo and the reaction mixture was diluted with diethyl ether, washed with satd aq NaCl, dried (Mg₂SO₄), and concentrated under a stream of nitrogen at 50° C. The crude product was purified by chromatography on a silica cartridge (80 g), 10% to 20% EtOAc in hexane. The desired fractions were concentrated under reduced pressure and dried under high vacuum, giving 2.8 g (70.5%) of the title compound. LC-MS m/z 252.2 (M+H)⁺ 1.16 (ret time)

### Intermediate 84: 1-methylethyl 2-(4-{[5-({[(1,1-dimethylethyl)(dimethyl)silyl]oxy}methyl)-2-pyridinyl]methyl}-1-piperazinyl)-3-pyridinecarboxylate

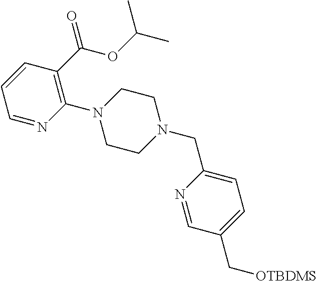 [SMILES: CC(C)OC(=O)c1cccnc1N1CCN(Cc2ccc(CO[Si](C)(C)C(C)(C)C)cn2)CC1]

Sodium triacetoxyborohydride (5.41 g, 25.5 mmol), 5-({[(1,1-dimethylethyl)(dimethyl)silyl]oxy}methyl)-2-pyridinecarbaldehyde (2.83 g, 11.26 mmol) and 1-methylethyl 2-(1-piperazinyl)-3-pyridinecarboxylate (2.12 g, 8.50 mmol) in THF (28.3 ml) were stirred for 12 h at 23° C. The reaction was extracted with EtOAc, and the organic phase was washed with 1M aq NaOH, water, and then satd aq NaCl, dried (MgSO₄), and concentrated under reduced pressure to afford 4.868 g (103%) of the title compound. LC-MS m/z 485.5 (M+H)⁺ 1.14 (ret time)

### Intermediate 85: 1-methylethyl 2-(4-{[5-(hydroxymethyl)-2-pyridinyl]methyl}-1-piperazinyl)-3-pyridinecarboxylate

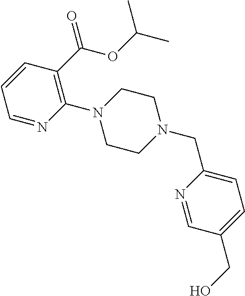 [SMILES: CC(C)OC(=O)c1cccnc1N1CCN(Cc2ccc(CO)cn2)CC1]

1-Methylethyl 2-(4-{[5-({[(1,1-dimethylethyl)(dimethyl)silyl]oxy}methyl)-2-pyridinyl]methyl}-1-piperazinyl)-3-pyridinecarboxylate (2.8437 g, 5.87 mmol) was dissolved in THF (10 ml), and triethylamine trihydrofluoride (0.955 ml, 5.87 mmol) was added and the resulting mixture was stirred at 23° C. for 5 h, concentrated under a stream of nitrogen at 50° C., dissolved in EtOAc, washed with satd aq NaHCO₃, dried (MgSO₄), and concentrated under a stream of nitrogen at 50° C., to afford 1.41 g (64%) of the title compound. LC-MS m/z 371.1 (M+H)⁺ 0.73 (ret time)

### Intermediate 86: 1-methylethyl 2-(4-{[5-(hydroxymethyl)-2-pyridinyl]methyl}-1-piperazinyl)-3-pyridinecarboxylate

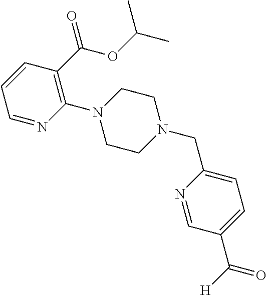 [SMILES: CC(C)OC(=O)c1cccnc1N1CCN(Cc2ccc(C=O)cn2)CC1]

1-Methylethyl 2-(4-{[5-(hydroxymethyl)-2-pyridinyl]methyl}-1-piperazinyl)-3-pyridine carboxylate (194.4 mg, 0.525 mmol) and activated MnO₂ (45.6 mg, 0.525 mmol) in dichloromethane (2.5 ml) were stirred at 23° C. for 16 h. More of activated MnO₂ (91.2 mg, 1.05 mmol) was added the mixture was stirred for 6 days. The reaction mixture was filtrated through a celite pad, and concentrated under a stream of nitrogen at 50° C. to afford 141 mg (73%) of the title compound. LC-MS m/z 369 (M+H)⁺ 0.78 (ret time)

### Intermediate 87: N-[(2-chloro-6-fluorophenyl)methyl]-N-{[5-({[(1,1-dimethylethyl) (dimethyl)silyl]oxy}methyl)-2-pyridinyl]methyl}ethanamine

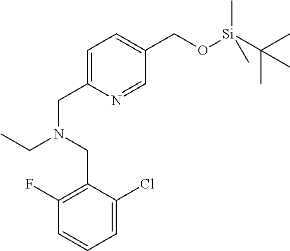 [SMILES: CCN(Cc1ccc(CO[Si](C)(C)C(C)(C)C)cn1)Cc1c(F)cccc1Cl]

5-({[(1,1-dimethylethyl)(dimethyl)silyl]oxy}methyl)-2-pyridinecarbaldehyde (220 mg, 0.875 mmol), N-[(2-chloro-6-fluorophenyl)methyl]ethanamine (328 mg, 1.750 mmol), sodium triacetoxyborohydride (556 mg, 2.63 mmol) in THF (4376 μl) were combined in a 10 ml vial. The mixture was stirred for 36 h. The reaction mixture was concentrated under a stream of nitrogen at 50° C. and dissolved in EtOAc, washed with NaOH 1N then water, dried (MgSO₄), and concentrated under a stream of nitrogen at 50° C. The crude product was purified by chromatography on a silica cartridge (40 g), eluting with a gradient from dichloromethane to 50% EtOAc in dichloromethane. The desired fractions were concentrated under reduced pressure and dried under high vacuum, giving 213 mg (58%) of the title compound. LC-MS m/z 423.0 (M+H)⁺ 0.78 (ret time)

### Intermediate 88: (6-{[[(2-chloro-6-fluorophenyl)methyl](ethyl)amino]methyl}-3-pyridinyl) methanol

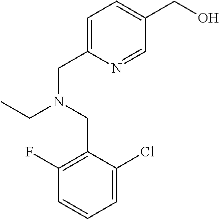 [SMILES: CCN(Cc1ccc(CO)cn1)Cc1c(F)cccc1Cl]

N-[(2-chloro-6-fluorophenyl)methyl]-N-{[5-({[(1,1-dimethylethyl)(dimethyl)silyl]oxy}methyl)-2-pyridinyl]methyl}ethanamine (213 mg, 0.503 mmol) was dissolved in THF (2.4 mL) and triethylamine trihydrofluoride (82 μl, 0.503 mmol) was added and the reaction was stirred at 23° C. for 16 h. The reaction was concentrated under a stream of nitrogen at 50° C. and dissolved in EtOAc, washed with satd aq NaHCO₃ three times, dried (MgSO₄) concentrated under a stream of nitrogen at 50° C. to afford 171.4 mg (110%) of the title compound LC-MS m/z 308.9 (M+H)⁺ 0.65 (ret time)

### Intermediate 89: 6-{[[(2-chloro-6-fluorophenyl)methyl](ethyl)amino]methyl}-3-pyridine carbaldehyde

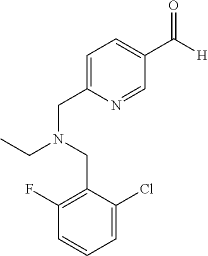 [SMILES: CCN(Cc1ccc(C=O)cn1)Cc1c(F)cccc1Cl]

(6-{[[(2-chloro-6-fluorophenyl)methyl](ethyl)amino]methyl}-3-pyridinyl)methanol (171.4 mg, 0.555 mmol) and activated MnO₂ (290 mg, 3.33 mmol) in dichloromethane (2.8 mL) were stirred at 23° C. for 7 h. Added 6 eq more of activated MnO₂ (290 mg, 3.33 mmol) and the mixture was stirred at 23° C. for 24 h. Filtration with celite and concentration under a stream of nitrogen at 50° C. afforded 108 mg (63%) of the title compound LC-MS m/z 307.1 (M+H)⁺ 0.57 (ret time) within an impure mixture. The crude product was used in the following reaction.

### Intermediate 90: Intermediate ester1 1-methylethyl 2-formylbenzoate

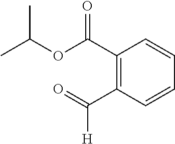

2-Formylbenzoic acid (1 g, 6.66 mmol) and SOCl₂ (1.458 ml, 19.98 mmol), were dissolved in dichloromethane (DCM) (10 ml) and 3 drops of DMF was added, and the mixture was stirred, and refluxed for 1 h. The volatiles were removed in vacuo and the residue was combined with pyridine (0.54 ml, 6.66 mmol) and 2-propanol (2.053 ml, 26.6 mmol), and heated to reflux for 1 h. The reaction was diluted with EtOAc and H₂O, shaken in a separatory funnel and the EtOAc was separated and concentrated. The crude product was purified on a silica cartridge (40 g) eluting at 40 mL/min with a gradient running from dichloromethane to 5% methanol in dichloromethane over 60 min to afford (1.28 g, 100%). LC-MS m/z 193.1 (M+H)⁺ 0.94 (ret time).

### Intermediate 91: 2 1-methylethyl 3-formylbenzoate

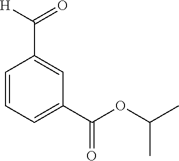

3-Formylbenzoic acid (0.5 g, 3.33 mmol) and SOCl₂ (0.729 ml, 9.99 mmol), were dissolved in dichloromethane (DCM) (10 ml) and 3 drops of DMF was added, and stirred, refluxed for 1 h. The volatiles were removed in vacuo and the residue was combined with pyridine (0.27 ml, 3.33 mmol) and 2-propanol (1.06 ml, 13.3 mmol), and heated to reflux for 24 h. The reaction was diluted with EtOAc and H₂O, shaken in a separatory funnel and the EtOAc was separated and concentrated. The crude product was purified on a silica cartridge (40 g) eluting at 40 mL/min with a gradient running from dichloromethane to 5% methanol in dichloromethane over 60 min to afford (74.6 mg, 12%). LC-MS m/z 193.1 (M+H)⁺ 0.94 (ret time).

### Intermediate 92: 3 1-methylethyl 4-formylbenzoate

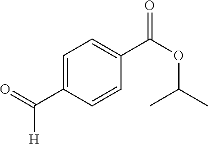

4-Formylbenzoic acid (1.5 g, 10 mmol) and SOCl₂ (2.2 ml, 30 mmol), were dissolved in dichloromethane (10 ml) and 3 drops of DMF was added, and stirred, and heated to dichloromethane reflux for 1 h. The volatiles were removed in vacuo and the residue was combined with pyridine (0.81 ml, 10 mmol) and 2-propanol (9.6 ml, 40 mmol), and heated to reflux for 24 h. The reaction was diluted with EtOAc and H₂O, shaken in a separatory funnel and the EtOAc was separated and concentrated. The crude product was purified on a silica cartridge (40 g) with a Combiflash Companion, eluting at 40 mL/min with a gradient running from dichloromethane to 5% methanol in dichloromethane over 60 min to afford the title compound (0.36 g, 19%) LC-MS m/z 193.1 (M+H)⁺ 0.94 (ret time).

### Intermediate 93: 1-methylethyl 4-formylbenzoate

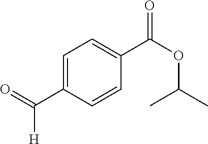

4-Formylbenzoic acid (1.5 g, 10 mmol) and SOCl₂ (2.2 ml, 30 mmol), were dissolved in dichloromethane (10 ml) and 3 drops of DMF was added, and stirred, and heated to dichloromethane reflux for 1 h. The volatiles were removed in vacuo and the residue was combined with pyridine (0.81 ml, 10 mmol) and 2-propanol (9.6 ml, 40 mmol), and heated to reflux for 24 h. The reaction was diluted with EtOAc and H₂O, shaken in a separatory funnel and the EtOAc was separated and concentrated. The crude product was purified on a silica cartridge (40 g) with a Combiflash Companion, eluting at 40 mL/min with a gradient running from dichloromethane to 5% methanol in dichloromethane over 60 min to afford the title compound (0.36 g, 19%) LC-MS m/z 193.1 (M+H)⁺ 0.94 (ret time).

### Intermediate 94: 2-cyano-N,N-dimethylbenzenesulfonamide

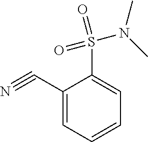

2-Cyanobenzenesulfonyl chloride (1.1 g, 5.46 mmol), 2M dimethylamine in THF (2.73 ml, 5.46 mmol), and triethylamine (0.76 ml, 5.46 mmol) were dissolved in dichloromethane (22 mL) at 4° C. The resulting solution was warmed to 23° C. and stirred for 2 h. The mixture was diluted with EtOAc and washed in succession with 1N aq NaOH, H₂O, and satd aq NaCl, dried (Na₂SO₄), and concentrated in vacuo. The crude product was purified on a silica cartridge (40 g) with a Combiflash Companion, eluting at 40 mL/min with a gradient running from 10% EtOAc/dichloromethane to 50% EtOAc/dichloromethane over 40 min. The desired fractions were concentrated in vacuo, to afford the title compound (531.8 mg, 46%) LC-MS m/z 211.1 (M+H)⁺ 0.61 (ret time).

### Intermediate 95: 3-cyano-N,N-dimethylbenzenesulfonamide

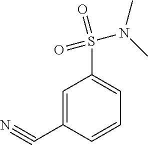

3-Cyanobenzenesulfonyl chloride (1.0 g, 4.96 mmol), 2 M dimethylamine in THF (2.48 ml, 4.96 mmol), triethylamine (0.691 ml, 4.96 mmol) in DCM (22 ml) were dissolved in dichloromethane (2.1 mL) at 4° C. The resulting solution was warmed to 23° C. and stirred for 2 h. The mixture was diluted with EtOAc and washed in succession with 1N aq NaOH, H₂O, and satd aq NaCl, dried (Na₂SO₄), and concentrated in vacuo. The crude product was purified on a silica cartridge (40 g) with a Combiflash Companion, eluting at 40 mL/min with a gradient running from 10% EtOAc/dichloromethane to 50% EtOAc/dichloromethane over 40 min. The desired fractions were concentrated in vacuo to afford the title compound (864 mg, 83%) LC-MS m/z 211.0 (M+H)⁺ 0.79 (ret time).

### Intermediate 96: 4-cyano-N,N-dimethylbenzenesulfonamide

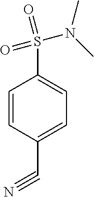

4-Cyanobenzenesulfonyl chloride (1.0 g, 4.96 mmol), dimethylamine in THF (4.96 ml, 9.92 mmol) and triethylamine (1.383 ml, 9.92 mmol) in DCM (18.46 ml) at 4° C. The resulting solution was warmed to 23° C. and stirred for 2 h. The mixture was diluted with EtOAc and washed in succession with 1N aq NaOH, H₂O, and satd aq NaCl, dried (Na₂SO₄), and concentrated in vacuo. The crude product was purified on a silica cartridge (40 g) with a Combiflash Companion, eluting at 40 mL/min with a gradient running from 10% EtOAc/dichloromethane to 50% EtOAc/dichloromethane over 40 min. The desired fractions were concentrated in vacuo to afford the title compound (885 mg, 97%) LC-MS m/z 211.0 (M+H)⁺ 0.79 (ret time).

### Intermediate 97: 2-Formyl-N,N-dimethylbenzenesufonamide

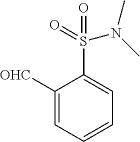

1 M DIBAL-H in DCM (8.1 mL, 8.1 mmol) was added dropwise to a solution of 2-cyano-N,N-dimethylbenzenesulfonamide (424.6 mg, 2.02 mmol) at −78° C. under argon. The reaction was warmed to 4° C. and stirred 2 h. Methanol (1 mL) was added and stirred 10 min and then 2M HCl in di-ethyl ether (6 mL) and dichloromethane (6 mL) were added and the mixture was stirred at 23° C. for 1 h. The mixture was concentrated in vacuo and partitioned between EtOAc and H₂O and the organic phase was concentrated to afford the title compound (112.3 mg, 26%) LC-MS m/z 214.1 (M+H)⁺ 0.66 (ret time) (70% purity)

### Intermediate 98: 3-Formyl-N,N-dimethylbenzenesulfonamide

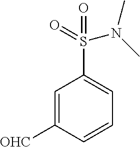

1 M DIBAL-H in DCM (20.87 ml, 20.87 mmol) was added dropwise to a solution of 3-cyano-N,N-dimethylbenzenesulfonamide (877.6 mg, 4.17 mmol) in dichloromethane (10 mL) at −78° C. under argon. The reaction was warmed to 4° C. and stirred 2 h. Methanol (1 mL) was added and stirred 10 min and then 2M HCl in di-ethyl ether (6 mL) and dichloromethane (6 mL) were added and the mixture was stirred at 23° C. for 1 h. The mixture was concentrated in vacuo and partitioned between EtOAc and H₂O and the organic phase was concentrated to afford the title compound (928.2 mg, 72.0%) LC-MS m/z 214.1 (M+H)⁺ 0.65 (ret time) (77% purity)

### Intermediate 99: 4-Formyl-N,N-dimethylbenzenesulfonamide

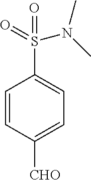

1M DIBAL-H in DCM (16.8 ml, 16.80 mmol) was added dropwise to a solution of 4-cyano-N,N-dimethylbenzenesulfonamide (885 mg, 4.21 mmol) in dichloromethane (5 mL) at −78° C. under argon. The reaction was warmed to 4° C. and stirred 2 h. Methanol (1 mL) was added and stirred 10 min and then 2M HCl in di-ethyl ether (6 mL) and dichloromethane (6 mL) were added and the mixture was stirred at 23° C. for 1 h. The mixture was concentrated in vacuo and partitioned between EtOAc and H₂O and the organic phase was concentrated to afford the title compound (804 mg, 57%). LC-MS m/z 214.1 (M+H)⁺0.74 (ret time) (77% purity)

### Intermediate 100: 2,5-bis(bromomethyl)pyrazine

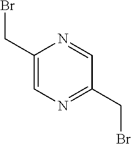

To a solution of 2,5-dimethylpyrazine (2.50 g, 23.12 mmol) in dry Carbontetrachloride (200 mL) was added NBS (8.23 g, 46.2 mmol) and benzoyl peroxide (0.560 g, 2.312 mmol) and heated to 75° C. for 73 hours. The reaction was cooled and filtered. The filtrate was concentrated and purified by silica column eluting with 0-20% EtOAc/Hexane to obtain the title compound (1.21 g, 19%). LC-MS m/z=266 (M+H), 0.67 minutes (retention time).

### Intermediate 101: 1,1-dimethylethyl 3,5-bis(bromomethyl)-1H-pyrazole-1-carboxylate

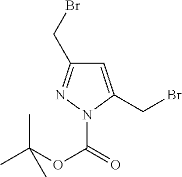 [SMILES: CC(C)(C)OC(=O)n1nc(CBr)cc1CBr]

To a solution of 1,1-dimethylethyl 3,5-dimethyl-1H-pyrazole-1-carboxylate (3.24 g, 16.51 mmol) in CCl4 (100 mL) was added NBS (6.17 g, 34.7 mmol) and benzoyl peroxide (1.120 g, 4.62 mmol) and heated to 75° C. for 3 hours. The reaction was cooled and solvent concentrated. The residue was dissolved in EtOAc, washed with saturated aqueous NaHCO₃(2×), brine, dried MgSO₄ and concentrated. The residue was purified by silica column eluting with 0-7% EtOAc/Hexane to obtain the title compound (0.339 g, 5%). LC-MS m/z=355 (M+H), 1.12 minutes (retention time).

### Intermediate 102: 1,1-dimethylethyl 3,5-bis{[[(3R)-1-(3-{[(3,3-dimethylbutanoyl)oxy]methyl}-2-pyridinyl)-3-pyrrolidinyl](ethyl)amino]methyl}-1H-pyrazole-1-carboxylate

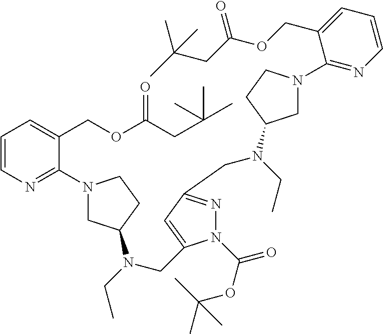 [SMILES: CCN(Cc1cc(CN(CC)[C@@H]2CCN(c3ncccc3COC(=O)CC(C)(C)C)C2)n(C(=O)OC(C)(C)C)n1)[C@@H]1CCN(c2ncccc2COC(=O)CC(C)(C)C)C1]

To a solution of {2-[(3R)-3-(ethylamino)-1-pyrrolidinyl]-3-pyridinyl}methyl 3,3-dimethylbutanoate (0.237 g, 0.604 mmol) and 1,1-dimethylethyl 3,5-bis(bromomethyl)-1H-pyrazole-1-carboxylate (0.107 g, 0.302 mmol) in Acetonitrile (7 mL) and Water (0.240 mL) was added potassium iodide (0.100 g, 0.604 mmol) and then potassium carbonate (0.125 g, 0.906 mmol). The reaction was stirred at room temperature for 21 hours. Solvent was concentrated and residue was dissolved in EtOAc, washed with water(2×), saturated aqueous NaHCO3, brine, dried MgSO4 concentrated. The residue was purified by silica column eluting with 100% EtOAc to obtain the title compound (0.138 g, 27%). LC-MS m/z=832 (M+H), 1.02 minutes (retention time).

### Intermediate 103: 1-methylethyl 2-[4-({4-[(ethylamino)methyl]phenyl}methyl)-1-piperazinyl]-3-pyridine carboxylate

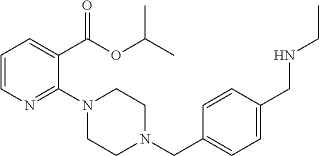 [SMILES: CCNCc1ccc(CN2CCN(c3ncccc3C(=O)OC(C)C)CC2)cc1]

To a solution of 1-methylethyl 2-{4-[(4-formylphenyl)methyl]-1-piperazinyl}-3-pyridinecarboxylate (3.43 g, 9.33 mmol) in Methanol (84 ml) was added 2M Ethylamine in THF (9.33 ml, 18.67 mmol) and acetic acid (0.107 ml, 1.867 mmol) and stirred at room temperature for 18 hours. Afterwards, sodium cyanoborohydride (2.053 g, 32.7 mmol) was added and stirred at room temperature for 22 hours. LCMS analysis indicated the reaction was incomplete so additional sodium cyanoborohydride (0.147 g, 2.334 mmol) was added and stirred for 22 hours. The solvent was concentrated and the residue was dissolved in EtOAc, washed with water (2×), brine, dried MgSO4, concentrated. The residue was purified by silica column eluting with 10-50% of a (10% bottle of 2N NH3/MeOH/DCM)/with DCM to obtain the title compound (1.22 g, 33%). LC-MS m/z=397 (M+H), 0.44 minutes (retention time).

### Intermediate 104: 1,1-dimethylethyl 6-{[4-(3-{[(1-methylethyl)oxy]carbonyl}-2-pyridinyl)-1-piperazinyl]methyl}-3,4-dihydro-2(1H)-isoquinolinecarboxylate

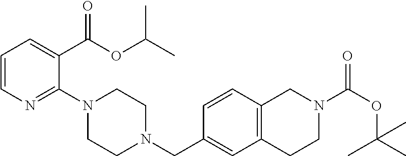 [SMILES: CC(C)OC(=O)c1cccnc1N1CCN(Cc2ccc3c(c2)CCN(C(=O)OC(C)(C)C)C3)CC1]

To a solution of 1-methylethyl 2-(1-piperazinyl)-3-pyridinecarboxylate (0.100 g, 0.401 mmol) and 6-Formyl-2-N-Boc-3,4-dihydroisoquinoline (0.131 g, 0.501 mmol) in Methanol (3 mL) was added Acetic Acid (7 μL, 0.122 mmol) and stirred at room temperature for 2 hours. Sodium cyanoborohydride (0.050 g, 0.802 mmol) was then added and stirred for 22 hours. Additional sodium cyanoborohydride (6.30 mg, 0.100 mmol) was added and stirred for 4 hours. The solvent was concentrated and the residue was dissolved in EtOAc, washed with water, brine, dried MgSO4, concentrated. The residue was purified by silica column eluting with 5-50% EtOAc/DCM to obtain the title compound (0.129 g, 65%). LC-MS m/z=495 (M+H), 0.99 minutes (retention time).

### Intermediate 105: 1-methylethyl 2-[4-(1,2,3,4-tetrahydro-6-isoquinolinylmethyl)-1-piperazinyl]-3-pyridine carboxylate-7hydrochloride

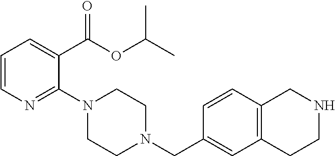 [SMILES: CC(C)OC(=O)c1cccnc1N1CCN(Cc2ccc3c(c2)CCNC3)CC1]

1,1-dimethylethyl 6-{[4-(3-{[(1-methylethyl)oxy]carbonyl}-2-pyridinyl)-1-piperazinyl]methyl}-3,4-dihydro-2(1H)-isoquinolinecarboxylate (0.129 g, 0.261 mmol) was dissolved in 4N HCl in 1,4 Dioxane (5.00 ml, 20.00 mmol) and stirred at room temperature for 18 hours. The solvent was concentrated to obtain the title compound (0.165 g, 97%). LC-MS m/z=395 (M+H), 0.48 minutes (retention time).

### Intermediate 106: 1-methylethyl 2-((3R)-3-{ethyl[(4-formylphenyl)methyl]amino}-1-pyrrolidinyl)-3-pyridinecarboxylate

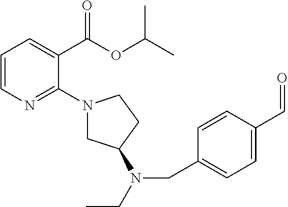 [SMILES: CCN(Cc1ccc(C=O)cc1)[C@@H]1CCN(c2ncccc2C(=O)OC(C)C)C1]

1-methylethyl2-[(3R)-3-(ethylamino)-1-pyrrolidinyl]-3-pyridinecarboxylate-2H Cl (0.500 g, 1.43 mmol) was dissolved in DCM and extracted with 1N NaOH to generate the freebase form of the molecule. The aqueous layer was backextracted 2×DCM and the combined organics were washed with brine, dried with MgSO4 and concentrated to give 0.325 g of the compound as a freebase oil. To a solution of 1-methylethyl 2-[(3R)-3-(ethylamino)-1-pyrrolidinyl]-3-pyridinecarboxylate (0.325 g, 1.172 mmol) in Acetone (5 mL) was added 4-(bromomethyl)benzaldehyde (0.210 g, 1.055 mmol) and K2CO3 (0.486 g, 3.52 mmol) and heated to 50° C. for 6 hours. The reaction was cooled and filtered and the solvent was concentrated. The residue was purified by silica column eluting with 0-30% EtOAc/DCM to obtain the title compound (0.379 g, 66%). LC-MS m/z=396 (M+H), 0.73 minutes (retention time).

### Intermediate 107: 1-methylethyl 2-{(3R)-3-[ethyl({4-[(ethylamino)methyl]phenyl}methyl)amino]-1-pyrrolidinyl}-3-pyridinecarboxylate

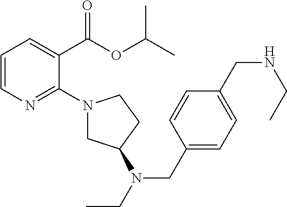 [SMILES: CCNCc1ccc(CN(CC)[C@@H]2CCN(c3ncccc3C(=O)OC(C)C)C2)cc1]

To a solution of 1-methylethyl 2-((3R)-3-{ethyl[(4-formylphenyl)methyl]amino}-1-pyrrolidinyl)-3-pyridinecarboxylate (0.379 g, 0.958 mmol) in Methanol (8.57 ml) was added 2M Ethylamine in THF (0.958 ml, 1.917 mmol) and acetic acid (0.055 ml, 0.958 mmol) and stirred at room temperature for 1 hour. Sodium cyanoborohydride (0.181 g, 2.87 mmol) was added and stirred at room temperature for 20 hours. The reaction was quenched with 1 ml water, and concentrated the solvent. The residue was dissolved in EtOAc, washed with water, back extracted aqueous with EtOAc (2×), washed combined organics with water, brine, dried MgSO4 to give the title compound (0.158 g, 38%). LC-MS m/z=425 (M+H), 0.60 minutes (retention time).

### Intermediate 108: 1-(3-{[(2-chloro-6-fluorophenyl)methyl]amino}propyl)-2-pyrrolidinone

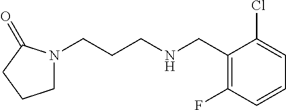 [SMILES: O=C1CCCN1CCCNCc1c(F)cccc1Cl]

To a solution of 2-chloro-6-fluorobenzaldehyde (0.043 g, 0.271 mmol) in 1,2-Dichloroethane (DCE) (2 mL) was added 1-(3-aminopropyl)-2-pyrrolidinone (0.046 mL, 0.325 mmol) and acetic acid (0.023 mL, 0.407 mmol), followed by sodium triacetoxyborohydride (0.086 g, 0.407 mmol) and stirred at room temperature for 1 hour. The solvent was concentrated and the residue was dissolved in DCM and washed with 1N NaOH. The layers were separated on a hydrophobic phase separator column, and the aqueous layer was extracted with DCM. The layers were separated as above, and the organic layer was concentrated to give the title compound (60 mg, 78%). LC-MS m/z=285 (M+H), 0.59 minutes (retention time) which was used without further purification.

Intermediate 109: (2-{4-[4-{[[(2-chloro-6-fluorophenyl)methyl](ethyl)amino]methyl}phenyl)methyl]-1-piperazinyl}-3-pyridinyl)methanol

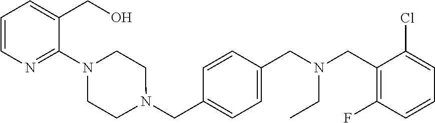 [SMILES: CCN(Cc1ccc(CN2CCN(c3ncccc3CO)CC2)cc1)Cc1c(F)cccc1Cl]

1-methylethyl 2-{4-[(4-{[[(2-chloro-6-fluorophenyl)methyl](ethyl)amino]methyl}phenyl)methyl]-1-piperazinyl}-3-pyridinecarboxylate (1.36 g, 2.52 mmol) was dissolved in Tetrahydrofuran (THF) (20 mL) and cooled in ice bath. Lithium aluminum hydride (0.105 g, 2.78 mmol) was added portion wise and then stirred at room temperature for 3 hours. LCMS analysis indicated the reaction was complete, so the reaction was cooled in an ice bath and 0.55 ml (0.22 ml/mmol) of saturated Na2SO4 solution was added with 20 ml THF and stirred for 30 minutes at room temperature. Afterwards, the white solids were filtered and the solvent concentrated. The residue was purified by silica column eluting with 0-5% MeOH/DCM to obtain the title compound (1.02 g, 82%). LC-MS m/z=483 (M+H), 0.55 minutes (retention time).

### Intermediate 110: 2-methyl-3-pyridinecarbaldehyde

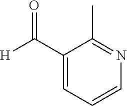

To a solution of 2-Methyl-3-hydroxymethylpyridine (0.300 g, 2.436 mmol) in Dichloromethane (DCM) (7 mL) was added manganese dioxide (0.847 g, 9.74 mmol) and stirred at room temperature for 18 hours. TLC analysis in 50% EtOAc/DCM indicated presence of minor starting material alcohol. More manganese dioxide (0.200 g, 2.301 mmol) was added and stirred at room temperature for 18 hours. TLC analysis indicated the reaction was complete. The reaction was filtered through a pad of Celite and washed with excess DCM. The solvent was concentrated and pumped on high vacuum. No purification was performed. ¹H NMR (400 MHz, DMSO-d₆) d ppm 2.80 (s, 3H) 7.47 (dd, J=7.65, 4.89 Hz, 1H) 8.18 (dd, J=7.78, 1.76 Hz, 1H) 8.68 (dd, J=4.77, 1.76 Hz, 1H) 10.29 (s, 1H)

### Intermediate 111: 1-methylethyl 2-{4-[(2-formylphenyl)methyl]-1-piperazinyl}-3-pyridinecarboxylate

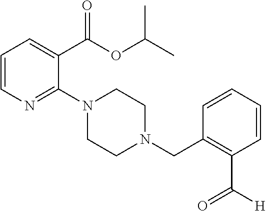 [SMILES: CC(C)OC(=O)c1cccnc1N1CCN(Cc2ccccc2C=O)CC1]

To a solution of 1-methylethyl 2-(4-{[2-(hydroxymethyl)phenyl]methyl}-1-piperazinyl)-3-pyridinecarboxylate (0.070 g, 0.189 mmol) in Dichloromethane (DCM) (3 mL) was added manganese dioxide (0.066 g, 0.758 mmol) and stirred at room temperature for 18 hours. TLC analysis in 50% EtOAc/DCM indicated reaction was mostly starting material. No change was observed after an additional 7 hours. Manganese dioxide (0.066 g, 0.758 mmol) was added and stirred for 18 hours. TLC indicated no progress. The solvent was removed and the reaction was dissolved in Chloroform (3.00 ml) and the reaction was heated to 50° C. for 2 hours and at room temperature for 2 days. TLC indicated the title compound was the major component. The reaction was filtered through a pad of Celite and the solvent was concentrated. The residue was purified by silica column eluting with 0-50% of EtOAc/DCM to obtain the title compound (30 mg, 43%). LC-MS m/z=368 (M+H), 0.71 minutes (retention time).

### Intermediate 112: Ethyl (2E)-3-(2-chloro-6-fluorophenyl)-2-propenoate

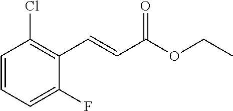

To a suspension of sodium hydride (0.717 g, 28.4 mmol) in dry Tetrahydrofuran (THF) (15 mL), was added triethyl phosphonoacetate (6.36 g, 28.4 mmol) in Tetrahydrofuran (THF) (7 mL) dropwise at 0° C. and stirred at 0° C. for 20 minuets. A solution of 2-chloro-6-fluorobenzaldehyde (3.00 g, 18.92 mmol) in Tetrahydrofuran (THF) (10 mL) was then added dropwise at 0° C. The reaction was then heated to 50° C. for 1 hour. Afterwards, it was cooled and quenched with 20 ml of saturated ammonium chloride. The layers were separated and the aqueous layer was extracted with 2× diethyl ether. The combined organics were washed with saturated. NaCl, dried MgSO4, and solvents were concentrated to give the title compound LC-MS m/z=229 (M+H), 1.18 minutes (retention time). Title compound was carried on crude for the preparation of 3-(2-chloro-6-fluorophenyl)-1-propanol.

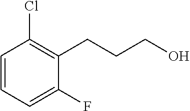 [SMILES: OCCCc1c(F)cccc1Cl]

### Intermediate 113: 3-(2-chloro-6-fluorophenyl)-1-propanol

Lithium aluminium hydride (2.154 g, 56.8 mmol) was suspended in Tetrahydrofuran (THF) (15 mL) and cooled to 0° C. Ethyl (2E)-3-(2-chloro-6-fluorophenyl)-2-propenoate (4.33 g, 18.92 mmol), in Tetrahydrofuran (THF) (25 mL) and added dropwise with dropping funnel to suspension and stirred at room temperature for 4 hours. Reaction was then cooled in ice bath and was quenched with 12 ml (0.22 ml/mmol of LAH) saturated Na2SO4. Solution turned yellow after addition of all Na2SO4 and white solids crashed out. Solids were filtered, washed with THF and concentrated solvent. The residue was dissolved in EtOAc, washed with water, brine, dried MgSO4, and concentrated. This residue was purified by silica column eluting with 0-10% of EtOAc/DCM to obtain the title compound (1.16 g, 32% for two reactions). LC-MS m/z=189 (M+H), 0.84 minutes (retention time).

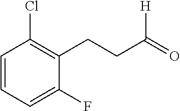 [SMILES: O=CCCc1c(F)cccc1Cl]

### Intermediate 114: 3-(2-chloro-6-fluorophenyl)propanal

3-(2-chloro-6-fluorophenyl)-1-propanol (0.150 g, 0.795 mmol) was dissolved in Dichloromethane (DCM) (3 mL) and cooled in ice bath. Then 4A molecular sieves were added followed by pyridinium chlorochromate (0.240 g, 1.113 mmol) and stirred at room temperature for 21 hours. The reaction was filtered through a pad of Celite and washed with excess DCM. The solvent was concentrated to give the title compound. LC-MS m/z=187 (M+H), 0.91 minutes (retention time). The title compound was carried on crude for the preparation of 1-methylethyl 2-{4-[(4-{[[3-(2-chloro-6-fluorophenyl)propyl](ethyl)amino]methyl}phenyl)methyl]-1-piperazinyl}-3-pyridinecarboxylate.

### Intermediate 115: 1-Methylethyl2-(4-{[4-(aminomethyl)phenyl]methyl}-1-piperazinyl)-3-Pyridinecarboxylate

Intermediate: 116: Methylethyl2-(4-{[4-(hydroxymethyl)phenyl]methyl}-1-piperazinyl)-3-pyridine carboxylate

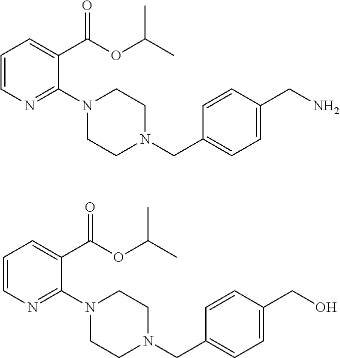 [SMILES: CC(C)OC(=O)c1cccnc1N1CCN(Cc2ccc(CN)cc2)CC1 ; CC(C)OC(=O)c1cccnc1N1CCN(Cc2ccc(CO)cc2)CC1]

To a solution of 1-methylethyl 2-{4-[(4-formylphenyl)methyl]-1-piperazinyl}-3-pyridinecarboxylate (1.03 g, 2.80 mmol) in Methanol (40 mL) was added Raney Nickel (0.300 g, 2.80 mmol) and ammonia hydroxide (10 mL, 2.80 mmol). The reaction was evacuated and purged with hydrogen 3 times and was stirred under hydrogen balloon for 18 hours. The reaction was filtered through Celite and the Celite was washed with methanol. All solvents were concentrated. The residue was purified by silica column eluting with 0-50% of a (10% bottle of 2N NH3/MeOH/DCM)/with DCM to obtain 1-methylethyl 2-(4-{[4-(aminomethyl)phenyl]methyl}-1-piperazinyl)-3-pyridinecarboxylate (0.712 g, 68%). LC-MS m/z=369 (M+H), 0.53 minutes (retention time) and 1-methylethyl 2-(4-{[4-(hydroxymethyl)phenyl]methyl}-1-piperazinyl)-3-pyridinecarboxylate (0.185 g, 17%). LC-MS m/z=370 (M+H), 0.67 minutes (retention time).

### Intermediate 117: 1-methylethyl 2-((3R)-3-{ethyl[(3-formylphenyl)methyl]amino}-1-pyrrolidinyl)-3-pyridinecarboxylate

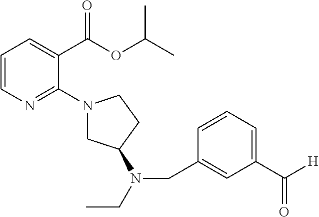 [SMILES: CCN(Cc1cccc(C=O)c1)[C@@H]1CCN(c2ncccc2C(=O)OC(C)C)C1]

1-methylethyl 2-[(3R)-3-(ethylamino)-1-pyrrolidinyl]-3-pyridinecarboxylate-2HCl (0.500 g, 1.43 mmol) was dissolved in DCM and extracted with 1N NaOH to generate the freebase form of the molecule. The aqueous layer was backextracted 4×DCM and the combined organics were washed with water, brine, dried with MgSO4 and concentrated to give 0.372 g of the compound as a freebase oil. To a solution of 1-methylethyl 2-[(3R)-3-(ethylamino)-1-pyrrolidinyl]-3-pyridinecarboxylate (0.372 g, 1.341 mmol) in Acetone (5 mL) was added 2-(bromomethylphenyl)benzaldehyde (0.267 g, 1.341 mmol) and K2CO3 (0.556 g, 4.02 mmol) and heated to 50° C. for 4 hours. The reaction was cooled and filtered and the solvent was concentrated. The residue was purified by silica column eluting with 0-30% EtOAc/DCM to obtain the title compound (0.383 g, 72%). LC-MS m/z=396 (M+H), 0.72 minutes (retention time).

### Intermediate 118: 1-methylethyl 2-{[4-(1-piperazinylmethyl)phenyl]methyl}-1-piperazinyl)-3-pyridinecarboxylate

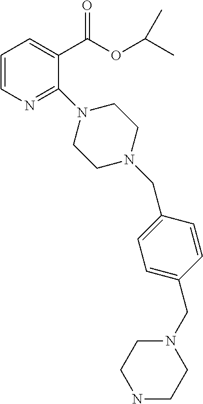 [SMILES: CC(C)OC(=O)c1cccnc1N1CCN(Cc2ccc(CN3CCNCC3)cc2)CC1]

To a round bottom bottle with 1-methylethyl 2-{4-[(4-formylphenyl)methyl]-1-piperazinyl}-3-pyridinecarboxylate (200 mg, 0.544 mmol) in Dichloromethane (DCM) (20 mL) with 1,1-dimethylethyl 1-piperazinecarboxylate (122 mg, 0.653 mmol) was added HOAc (32.7 mg, 0.544 mmol). The result solution was stirred for 2 hr. Na(OAc)₃BH (233 mg, 1.089 mmol) was added into the solution and stirred for another 12 hr. H₂O (10 mL) and DCM (10 mL) were added and the result solution was separated by Phase Separator. The water layer was washed with DCM (10 mL). Combined the organic layer and removed the solvent. MeOH (10 mL) and Dioxane (20 mL) were added to the residue with HCl (29.6 mg, 10.89 mmol). The result solution was heated at 60° C. for 1 hr. Removed the solvent to get 120 mg (50.4%) of the desired product. LC/MS: m/z=438.1[M+H]⁺, Ret. Time: 0.57 min

### Compound Examples

### Example 1

### 1-Methylethyl 2-{4-[(5-ethyl-2-thienyl)methyl]-1-piperazinyl}-3-pyridinecarboxylate

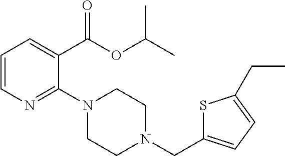 [SMILES: CCc1ccc(CN2CCN(c3ncccc3C(=O)OC(C)C)CC2)s1]

To a solution of isopropyl 2-(piperazin-1-yl)nicotinate hydrochloride (50 mg, 0.175 mmol) in EtOAc (10 mL) was added Et₃N (35 mg, 0.35 mmol). The mixture was stirred at room temperature for 30 min. The solid was filtered; the filtrate was concentrated to dryness. To this residue was added DCE (10 mL), 5-ethyl-2-thiophene carboxaldehyde (30 mg, 0.21 mmol), HOAc (16 mg, 0.263 mmol), NaBH(OAc)₃ (75 mg, 0.35 mmol) and the resulting mixture was stirred at room temperature for 3 h. The mixture was filtered; the solid was washed with DCE (5 mL). The filtrates were concentrated under reduced pressure. The residue was purified on Pre-TLC eluting with 20% EtOAc of Hexane to give the title compound (49 mg, 75%) as yellow oil. LC-MS m/z 374.2 (M+H)⁺, 1.94 min (ret time); ¹H NMR (400 MHz, CDCl₃) δ 8.26-8.24 (m, 1H), 7.94-7.91 (m, 1H), 6.74-6.61 (m, 3H), 5.19-5.16 (m, 1H), 3.72 (s, 2H), 3.48-3.46 (m, 4H), 2.82 (q, J=7.6, 14.8 Hz, 2H), 2.64-2.63 (m, 4H), 1.35 (d, J=6.4 Hz, 6H), 1.30 (t, J=7.6 Hz, 3H).

### Example 2

### 1-Methylethyl 2-{4-[(4,5-dimethyl-2-thienyl)methyl]-1-piperazinyl}-3-pyridinecarboxylate

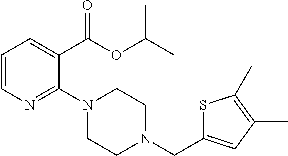 [SMILES: Cc1cc(CN2CCN(c3ncccc3C(=O)OC(C)C)CC2)sc1C]

Following the general procedure of 1-Methylethyl 2-{4-[(5-ethyl-2-thienyl)methyl]-1-piperazinyl}-3-pyridinecarboxylate

Isopropyl 2-(piperazin-1-yl)nicotinate hydrochloride (100 mg, 0.35 mmol) in EtOAc (10 mL), Et₃N (70 mg, 0.70 mmol), 4,5-dimethyl-2-thiophene carboxaldehyde (59 mg, 0.42 mmol), HOAC (32 mg, 0.53 mmol) and NaBH(OAc)₃ (148 mg, 0.70 mmol) were reacted to give the title compound (94 mg, 72%) as yellow oil. LC-MS m/z 374.2 (M+H)⁺, 1.93 min (ret time); ¹H NMR (400 MHz, CDCl₃) δ 8.26-8.24 (m, 1H), 7.94-7.91 (m, 1H), 6.72-6.69 (m, 1H), 6.60 (s, 1H), 5.19-5.16 (m, 1H), 3.64 (s, 2H), 3.46 (t, J=5.2 Hz, 4H), 2.59 (t, J=5.2 Hz, 4H), 2.30 (s, 3H), 2.08 (s, 3H), 1.34 (d, J=6.4 Hz, 6H).

### Example 3

### 1-Methylethyl 2-{4-[(4-ethylphenyl)methyl]-1-piperazinyl}-3-pyridinecarboxylate

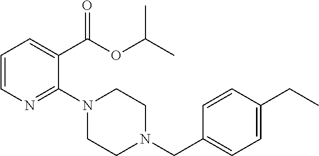 [SMILES: CCc1ccc(CN2CCN(c3ncccc3C(=O)OC(C)C)CC2)cc1]

To a suspension of isopropyl 2-(piperazin-1-yl)nicotinate hydrochloride (100 mg, 0.35 mmol) in THF (10 mL) was added Et₃N (53 mg, 0.53 mmol). The reaction mixture was stirred at room temperature for 10 min. 4-Ethylbenzaldehyde (56 mg, 0.42 mmol) was added, followed by NaBH(OAc)₃ (148 mg, 0.70 mmol). The resulting mixture was stirred at room temperature for 1 h. The suspension was filtered, the solid cake was washed with THF (5 mL), and the filtrate was concentrated under reduced pressure. The residue was purified by column chromatography (300-400 um, 50 g) eluting with 200 mL 10% EtOAc of petroleum ether to give the title compound (105 mg, 82%) as yellow oil. LC-MS m/z 368.2 (M+H)⁺, 2.23 min (ret time); ¹H NMR (400 MHz, CDCl₃) δ 8.27-8.24 (m, 1H), 7.93-7.90 (m, 1H), 7.27-7.15 (m, 4H), 6.72-6.69 (m, 1H), 5.19-5.16 (m, 1H), 3.55 (s, 2H), 3.45 (t, J=4.8 Hz, 4H), 2.67-2.61 (q, J=7.6, 14.8 Hz, 2H), 2.58 (t, J=4.8 Hz, 4H), 1.33 (d, J=6.4 Hz, 6H), 1.23 (t, J=7.6 Hz, 3H).

### Example 4

### 1-Methyl ethyl 2-{4-[(2-ethylphenyl)methyl]-1-piperazinyl}-3-pyridinecarboxylate

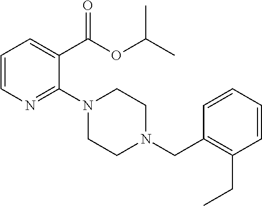 [SMILES: CCc1ccccc1CN1CCN(c2ncccc2C(=O)OC(C)C)CC1]

To a suspension of isopropyl 2-(piperazin-1-yl)nicotinate hydrochloride (100 mg, 0.35 mmol) in THF (10 mL) was added Et₃N (53 mg, 0.53 mmol). The reaction mixture was stirred at room temperature for 30 min. The mixture was filtered; the filtrate was concentrated to dryness. To this residue was added DCE (10 mL), 2-ethylbenzaldehyde (56 mg, 0.42 mmol), HOAc (30 mg, 0.5 mmol) and NaBH(OAc)₃ (148 mg, 0.70 mmol). The resulting mixture was stirred at room temperature for 16 h. The mixture was filtered; the solid was washed with DCE (5 mL). The filtrates were concentrated under reduced pressure. The residue was purified by column chromatography (300-400 um, 50 g) eluting with 200 mL 10% EtOAc of petroleum ether to give the title compound (97 mg, 76%) as yellow oil. LC-MS m/z 368.2 (M+H)⁺, 2.31 min (ret time); ¹H NMR (400 MHz, CDCl₃) δ 8.26-8.24 (m, 1H), 7.92-7.90 (m, 1H), 7.30-7.20 (m, 4H), 6.72-6.69 (m, 1H), 5.20-5.14 (m, 1H), 3.53 (s, 2H), 3.43 (t, J=4.8 Hz, 4H), 2.75 (q, J=7.6, 14.8 Hz, 2H), 2.56 (t, J=4.8 Hz, 4H), 1.35 (d, J=6.4 Hz, 6H), 1.23 (t, J=7.6 Hz, 3H).

### Example 5

### 1-Methylethyl 2-{methyl[(3S)-1-(phenylmethyl)-3-pyrrolidinyl]amino}-3-pyridine carboxylate hydrochloride

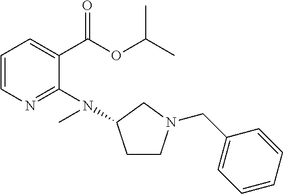 [SMILES: CC(C)OC(=O)c1cccnc1N(C)[C@H]1CCN(Cc2ccccc2)C1]

A mixture of isopropyl 2-chloronicotinate (330 mg, 1.653 mmol) and (S)-1-benzyl-N-methylpyrrolidin-3-amine (630 mg, 3.306 mmol) in a microwave vial was heated in microwave for 2 h at 120° C. The crude product was purified by silica gel column to give the free base product as yellow oil (364 mg, 62%). It was re-dissolved in the solution of HCl in dioxane and stirred for 5 min to give the title product. LC-MS m/z 354 (M+H)⁺, 1.15 min (ret time); ¹H NMR (400 MHz, CDCl₃) δ 13.11 (s, 1H), 8.35-8.33 (d, J=5.6 Hz, 1H), 8.25-8.23 (d, J=7.2 Hz, 1H), 7.75-7.74 (d, J=6.0 Hz, 2H), 7.44-7.42 (m, 3H), 7.071 (t, J=8.9 Hz, 1H), 5.70-5.66 (m, 1H), 5.25-5.22 (m, 1H), 4.46-4.32 (m, 3H), 3.73-3.70 (m, 1H), 3.61-3.58 (m, 1H), 3.19 (s, 3H), 3.10-3.07 (m, 1H), 2.80-2.76 (m, 1H), 2.53-2.50 (m, 1H), 1.40-1.30 (dd, J=8.4 Hz, 6H)

### Example 6

### 1-Methylethyl2-{methyl[(3R)-1-(phenylmethyl)-3-pyrrolidinyl]amino}-3-pyridine carboxylate hydrochloride

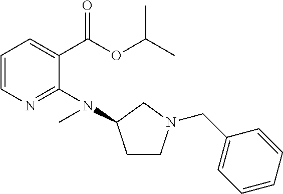 [SMILES: CC(C)OC(=O)c1cccnc1N(C)[C@@H]1CCN(Cc2ccccc2)C1]

A mixture of isopropyl 2-chloronicotinate (360 mg, 1.803 mmol) and (R)-1-benzyl-N-methylpyrrolidin-3-amine (686 mg, 3.607 mmol) in a microwave vial was heated in a microwave for 2 h at 120° C. The reaction mixture was diluted with DCM, washed with water and brine, dried over Na₂SO₄ and concentrated. The crude product was purified by silica gel column eluting with 10:1 ratio of ethyl acetate in petroleum ether to give the free base product as yellow oil (349 mg, 55%). It was then re-dissolved in the solution of HCl in dioxane (2 mL) and stirred for 5 min to obtain the title product. Solvent was removed to give the title product (349 mg, 55%) as white solid. LC-MS m/z 354 (M+H)⁺, 1.15 min (ret time); ¹H NMR (400 MHz, CDCl₃) δ 13.11 (s, 1H), 8.34-8.32 (d, J=5.6 Hz, 1H), 8.23-8.21 (d, J=6 Hz, 1H), 7.75-7.73 (d, J=6.0 Hz, 2H), 7.44-7.42 (m, 3H), 7.06-7.03 (m, 1H), 5.71-5.63 (m, 1H), 5.25-5.21 (m, 1H), 4.45-4.32 (m, 3H), 3.74-3.70 (m, 1H), 3.62-3.57 (m, 1H), 3.18 (s, 3H), 3.08-3.03 (m, 1H), 2.79-2.75 (m, 1H), 2.52-2.48 (m, 1H), 1.39-1.35 (dd, J=6 Hz, 6H).

### Example 7

### 1-Methylethyl 2-((3S)-3-{[(5-ethyl-2-thienyl)methyl]amino}-1-pyrrolidinyl)-3-pyridinecarboxylate

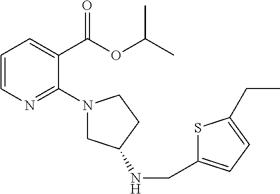 [SMILES: CCc1ccc(CN[C@H]2CCN(c3ncccc3C(=O)OC(C)C)C2)s1]

A mixture of 1-Methylethyl 2-[(3S)-3-amino-1-pyrrolidinyl]-3-pyridinecarboxylate (168 mg, 0.675 mmol) and 5-ethylthiophene-2-carbaldehyde (63 mg, 0.45 mmol) in THF (1 mL) was stirred at RT for 30 min, then NaBH(OAc)₃ (429 mg, 2.02 mmol) was added. The mixture was stirred at room temperature overnight. The crude product was purified to obtain the title compound (80 mg, 32%) as yellow oil. LC-MS m/z 374 (M+H)⁺, 2.09 min (ret time); ¹H NMR (400 MHz, CDCl₃) δ 8.24-8.22 (m, 1H), 7.84-7.81 (m, 1H), 6.71-6.70 (m, 1H), 6.61-6.58 (m, 2H), 5.22-5.16 (m, 1H), 3.94 (s, 2H), 3.60-3.45 (m, 4H), 3.28-3.24 (m, 1H), 2.82-2.76 (m, 2H), 2.18-2.10 (m, 1H), 1.87-1.79 (m, 1H), 1.67 (s, 1H), 1.36-1.35 (d, J=6.4 Hz, 6H), 1.27 (t, J=7.6 Hz, 3H).

### Example 8

### 1-Methylethyl 2-((3S)-3-{[(4,5-dimethyl-2-thienyl)methyl]amino}-1-pyrrolidinyl)-3-pyridinecarboxylate

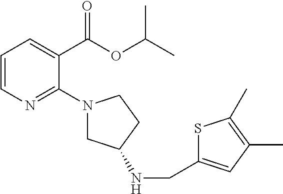 [SMILES: Cc1cc(CN[C@H]2CCN(c3ncccc3C(=O)OC(C)C)C2)sc1C]

A mixture of 1-Methylethyl 2-[(3S)-3-amino-1-pyrrolidinyl]-3-pyridinecarboxylate (168 mg, 0.675 mmol) and 4,5-dimethylthiophene-2-carbaldehyde (63 mg, 0.45 mmol) in THF (1 mL) was stirred at RT for 30 min. Then NaBH(OAc)₃ (429 mg, 2.02 mmol) was added. The mixture was stirred at room temperature overnight. The crude product was purified to obtain the title compound (50 mg, 30%) as yellow oil. LC-MS m/z 374 (M+H)⁺, 2.08 min (ret time); ¹H NMR (400 MHz, CDCl₃) δ 8.243-8.22 (m, 1H), 7.83-7.81 (m, 1H), 6.60-6.6.57 (m, 2H), 5.22-5.16 (m, 1H), 3.88 (s, 2H), 3.59-3.44 (m, 4 H), 3.27-3.23 (m, 1H), 2.28 (s, 3H), 2.17-2.10 (m, 1H), 2.06 (s, 3H), 1.86-1.77 (m, 1 H), 1.62 (s, 1H), 1.36-1.34 (dd, J=1.2 Hz, 1.6 Hz, 6H).

### Example 9

### 1-Methylethyl 2-((3S)-3-{[(3-ethylphenyl)methyl]amino}-1-pyrrolidinyl)-3-pyridine carboxylate

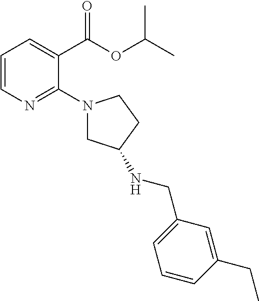 [SMILES: CCc1cccc(CN[C@H]2CCN(c3ncccc3C(=O)OC(C)C)C2)c1]

A mixture of 1-Methylethyl 2-[(3S)-3-amino-1-pyrrolidinyl]-3-pyridinecarboxylate (249 mg, 1 mmol) and 3-ethylbenzaldehyde (89 mg, 0.67 mmol) in THF (1 mL) was stirred for 10 min. Then NaBH(OAc)₃ (424 mg, 2 mmol) was added. The mixture was stirred at room temperature overnight. Water (10 mL) was added to the mixture and it was extracted with ethyl acetate (3×10 mL). The combined organic layer was dried over Na₂SO₄ and concentrated to obtain the crude product. It was purified by HPLC to obtain the title compound (100 mg, 27%) as yellow oil. LC-MS m/z 368 (M+H)⁺, 2.09 min (ret time); ¹H NMR (400 MHz, CDCl₃) δ 8.25-8.23 (m, 1H), 7.84-7.82 (m, 1H), 7.26-7.21 (m, 1H), 7.15-7.07 (m, 3H), 6.61-6.58 (m, 1H), 5.24-5.15 (m, 1H), 3.80 (s, 2H), 3.61-3.41 (m, 4H), 3.31-3.27 (m, 1H), 2.66-2.60 (m, 2H), 2.19-2.11 (m, 1H), 1.88-1.80 (m, 1H), 1.54 (s, 1H), 1.36-1.35 (d, J=6 Hz, 6H), 1.22 (t, J=8 Hz, 3H).

### Example 10

### 1-Methylethyl 2-((3S)-3-{[(4-ethylphenyl)methyl]amino}-1-pyrrolidinyl)-3-pyridine carboxylate

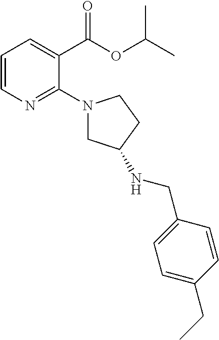 [SMILES: CCc1ccc(CN[C@H]2CCN(c3ncccc3C(=O)OC(C)C)C2)cc1]

To a solution of 1-Methylethyl 2-[(3S)-3-amino-1-pyrrolidinyl]-3-pyridinecarboxylate (137 mg, 0.55 mmol) in acetone (5 mL) was added 1-(bromomethyl)-4-ethylbenzene (100 mg, 0.5 mmol) and K₂CO₃ (104 mg, 0.75 mmol). The mixture was heated at 60° C. for 3 h. It was filtered and the filtrate was concentrated to obtain the crude product. It was purified by Prep HPLC to obtain the title compound (50 mg, 25%) as yellow oil. LC-MS m/z 368 (M+H)⁺, 2.10 min (ret time); ¹H NMR (400 MHz, CDCl₃) δ 8.23-8.21 (m, 1H), 7.84-7.82 (m, 1H), 7.30-7.26 (m, 2H), 7.17-7.15 (m, 2H), 6.62-6.59 (m, 1H), 5.21-5.15 (m, 1H), 3.90-3.82 (m, 2H), 3.65-3.61 (m, 1H), 3.52-3.41 (m, 4H), 2.98 (s, 1H), 2.65-2.59 (m, 2H), 2.23-2.15 (m, 1H), 2.02-1.93 (m, 1H), 1.36-1.34 (d, J=6 Hz, 6H), 1.21 (t, J=8 Hz, 3H).

### Example 11

### 1-Methylethyl 2-((3S)-3-{[(2-ethylphenyl)methyl]amino}-1-pyrrolidinyl)-3-pyridinecarboxylate

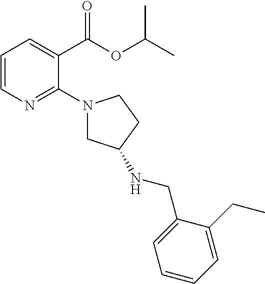 [SMILES: CCc1ccccc1CN[C@H]1CCN(c2ncccc2C(=O)OC(C)C)C1]

To a solution of 1-Methylethyl 2-[(3S)-3-amino-1-pyrrolidinyl]-3-pyridinecarboxylate (137 mg, 0.55 mmol) in acetone (5 mL) was added 1-(bromomethyl)-2-ethylbenzene (100 mg, 0.5 mmol) and K₂CO₃ (104 mg, 0.75 mmol). The mixture was heated at 60° C. for 3 h. It was filtered and the filtrate was concentrated to obtain the crude product. It was purified by Prep HPLC to obtain the title compound (30 mg, 15%) as yellow oil. LC-MS m/z 368 (M+H)⁺, 2.12 min (ret time); ¹H NMR (400 MHz, CDCl₃) δ 8.24-8.23 (m, 1H), 7.84-7.82 (m, 1H), 7.30-7.12 (m, 4H), 6.61-6.58 (m, 1H), 5.23-5.16 (m, 1 H), 3.81 (s, 2H), 3.63-3.44 (m, 4H), 3.30-3.26 (m, 1H), 2.70-2.64 (m, 2H), 2.20-2.13 (m, 1H), 1.90-1.82 (m, 1H), 1.52 (m, 1H), 1.36-1.35 (d, J=6 Hz, 6H), 1.20 (t, J=7.6 Hz, 3H).

### Example 12

### 1-Methylethyl 2-{(3S)-3-[[(5-ethyl-2-thienyl)methyl](methyl)amino]-1-pyrrolidinyl}-3-pyridinecarboxylate

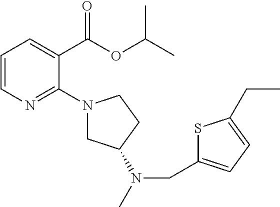 [SMILES: CCc1ccc(CN(C)[C@H]2CCN(c3ncccc3C(=O)OC(C)C)C2)s1]

A solution of 1-Methylethyl 2-[(3S)-3-(methylamino)-1-pyrrolidinyl]-3-pyridinecarboxylate (131 mg, 0.5 mmol) and 5-ethylthiophene-2-carbaldehyde (63 mg, 0.45 mmol) in THF (1 mL) was stirred at RT for 30 min. Then NaBH(OAc)₃ (319 mg, 1.5 mmol) was added. The mixture was stirred at room temperature overnight. The crude product was purified to obtain the title compound (40 mg, 21%) as yellow oil. LC-MS m/z 388 (M+H)⁺, 2.23 min (ret time); ¹H NMR (400 MHz, CDCl₃) δ 8.25-8.23 (m, 1H), 7.83-7.81 (m, 1H), 6.69-6.69 (m, 1H), 6.61-6.58 (m, 2H), 5.23-5.16 (m, 1 H), 3.77 (s, 2H), 3.66-3.36 (m, 4H), 3.13-3.05 (m, 1H), 2.82-2.77 (m, 2H), 2.28 (s, 3 H), 2.21-2.15 (m, 1H), 1.97-1.89 (m, 1H), 1.38-1.34 (dd, J=6.4 Hz, 6H), 1.28 (m, J=7.6 Hz, 3H).

### Example 13

### 1-Methylethyl 2-{(3S)-3-[[(4,5-dimethyl-2-thienyl)methyl](methyl)amino]-1-pyrrolidinyl}-3-pyridinecarboxylate

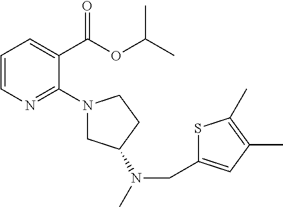 [SMILES: Cc1cc(CN(C)[C@H]2CCN(c3ncccc3C(=O)OC(C)C)C2)sc1C]

A solution of 1-Methylethyl 2-[(3S)-3-(methylamino)-1-pyrrolidinyl]-3-pyridinecarboxylate (131 mg, 0.5 mmol) and 4,5-dimethylthiophene-2-carbaldehyde (63 mg, 0.45 mmol) in THF (1 mL) was stirred at RT for 30 min. Then NaBH(OAc)₃ (319 mg, 1.5 mmol) was added. The mixture was stirred at room temperature overnight. The crude product was purified to obtain the title compound (60 mg, 31%) as yellow oil. LC-MS m/z 388 (M+H)⁺, 2.22 min (ret time); ¹H NMR (400 MHz, CDCl₃) δ 8.25-8.23 (m, 1H), 7.83-7.81 (m, 1H), 6.61-6.57 (m, 2H), 5.23-5.16 (m, 1H), 3.72 (s, 2H), 3.66-3.35 (m, 4H), 3.12-3.04 (m, 1H), 2.28 (s, 6H), 2.20-2.14 (m, 1H), 2.07 (s, 3H), 1.96-1.85 (m, 1H), 1.37-1.34 (dd, J=6.4 Hz, 6H).

### Example 14

### 1-Methylethyl 2-{(3S)-3-[[(3-ethylphenyl)methyl](methyl)amino]-1-pyrrolidinyl}-3-pyridinecarboxylate

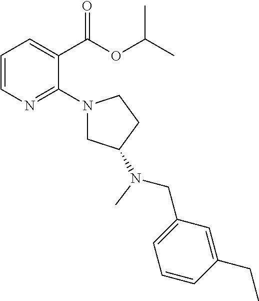 [SMILES: CCc1cccc(CN(C)[C@H]2CCN(c3ncccc3C(=O)OC(C)C)C2)c1]

A solution of 1-Methylethyl 2-[(3S)-3-(methylamino)-1-pyrrolidinyl]-3-pyridinecarboxylate (270 mg, 1.03 mmol) and 3-ethylbenzaldehyde (125 mg, 0.93 mmol) in THF (1 mL) was stirred for 10 min. Then NaBH(OAc)₃ (594 mg, 2.79 mmol) was added. The mixture was stirred at room temperature overnight. Water (10 mL) was added to the reaction mixture and it was extracted with ethyl acetate (3×10 mL). The combined organic layer was dried over Na₂SO₄ and concentrated to obtain the crude product. It was purified by Prep HPLC to obtain the title compound (200 mg, 51%) as yellow oil. LC-MS m/z 382 (M+H)⁺, 2.28 min (ret time); ¹H NMR (400 MHz, CDCl₃) δ 8.25-8.24 (m, 1H), 7.84-7.82 (m, 1H), 7.26-7.07 (m, 4H), 6.61-6.58 (m, 1 H), 5.22-5.17 (m, 1H), 3.68-3.40 (m, 6H), 3.11-3.04 (m, 1H), 3.66-2.60 (m, 2H), 2.18-2.17 (m, 4H), 2.00-1.90 (m, 1H), 1.37-1.33 (m, 6H), 1.25-1.21 (m, 3H).

### Example 15

### 1-Methylethyl 2-{(3S)-3-[[(4-ethylphenyl)methyl](methyl)amino]-1-pyrrolidinyl}-3-pyridinecarboxylate

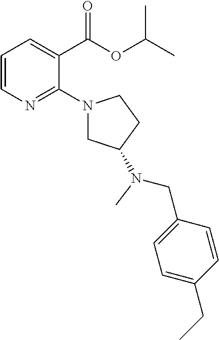 [SMILES: CCc1ccc(CN(C)[C@H]2CCN(c3ncccc3C(=O)OC(C)C)C2)cc1]

To a solution of 1-Methylethyl 2-[(3S)-3-(methylamino)-1-pyrrolidinyl]-3-pyridinecarboxylate (131 mg, 0.5 mmol) in acetone (5 mL) was added 1-(bromomethyl)-4-ethylbenzene (100 mg, 0.5 mmol) and K₂CO₃ (104 mg, 0.75 mmol). The mixture was heated at 60° C. for 3 h. The reaction mixture was filtered and the filtrate was concentrated to obtain the crude product. It was purified by Prep HPLC to obtain the title compound (70 mg, 37%) as yellow oil. LC-MS m/z 382 (M+H)⁺, 2.28 min (ret time); ¹H NMR (400 MHz, CDCl₃) δ 8.25-8.24 (m, 1H), 7.84-7.81 (m, 1H), 7.26-7.13 (m, 4H), 6.62-6.59 (m, 1H), 5.22-5.16 (m, 1H), 3.67-3.39 (m, 6H), 3.11-3.03 (m, 1H), 2.66-2.60 (m, 2H), 2.22-2.17 (m, 4H), 1.99-1.79 (m, 1H), 1.37-1.33 (dd, J=6.4 Hz, 6H), 1.22 (t, J=8 Hz, 3H).

### Example 16

### 1-Methylethyl 2-{(3S)-3-[[(2-ethylphenyl)methyl](methyl)amino]-1-pyrrolidinyl}-3-pyridinecarboxylate

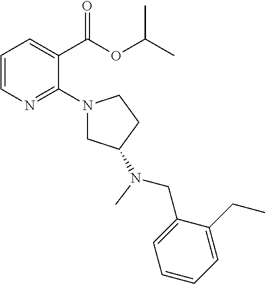 [SMILES: CCc1ccccc1CN(C)[C@H]1CCN(c2ncccc2C(=O)OC(C)C)C1]

To a solution of 1-Methylethyl 2-[(3S)-3-(methylamino)-1-pyrrolidinyl]-3-pyridinecarboxylate (131 mg, 0.5 mmol) in acetone (5 mL) was added 1-(bromomethyl)-2-ethylbenzene (100 mg, 0.5 mmol) and K₂CO₃ (104 mg, 0.75 mmol). The mixture was heated at 60° C. for 3 h. The reaction mixture was filtered and the filtrate was concentrated to obtain the crude product. It was purified by Prep HPLC with a basic buffer condition to obtain the title compound (120 mg, 63%) as yellow oil. LC-MS m/z 382 (M+H)⁺, 2.33 min (ret time); ¹H NMR (400 MHz, CDCl₃) δ 8.17-8.16 (m, 1H), 7.76-7.74 (m, 1H), 7.22-7.03 (m, 4H), 6.53-6.50 (m, 1H), 5.19-5.08 (m, 1 H), 3.61-3.35 (m, 6H), 3.03-2.96 (m, 1H), 2.68-2.63 (m, 2H), 2.15-2.09 (m, 1H), 2.04 (s, 3H), 1.95-1.85 (m, 1H), 1.28-1.24 (dd, J=6.4 Hz, 6H), 1.12 (t, J=8 Hz, 3H).

### Example 17

### 1-Methylethyl 2-((3S)-3-{ethyl[(5-ethyl-2-thienyl)methyl]amino}-1-pyrrolidinyl)-3-pyridinecarboxylate

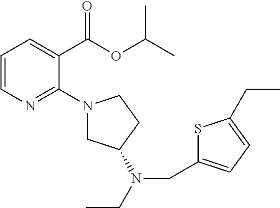 [SMILES: CCc1ccc(CN(CC)[C@H]2CCN(c3ncccc3C(=O)OC(C)C)C2)s1]

A solution of 1-Methylethyl 2-[(3S)-3-(ethylamino)-1-pyrrolidinyl]-3-pyridinecarboxylate (125 mg, 0.45 mmol), 5-ethylthiophene-2-carbaldehyde (57 mg, 0.41 mmol) in THF (1 mL) was stirred at RT for 30 min. Then NaBH(OAc)₃ (287 mg, 1.35 mmol) was added. The mixture was stirred at room temperature overnight. The crude product was purified to obtain the title compound (50 mg, 28%) as yellow oil. LC-MS m/z 402 (M+H)⁺, 2.33 min (ret time); ¹H NMR (400 MHz, CDCl₃) δ 8.24-8.23 (m, 1H), 7.82-7.80 (m, 1H), 6.69-6.68 (m, 1H), 6.60-6.57 (m, 2H), 5.22-5.16 (m, 1H), 3.87 (s, 2H), 3.64-3.32 (m, 5H), 2.82-2.76 (m, 2H), 2.69-2.62 (m, 2H), 2.19-2.13 (m, 1H), 1.95-1.85 (m, 1H), 1.37-1.33 (dd, J=6.4 Hz, 6H), 1.28 (t, J=7.6 Hz, 3H), 1.07 (t, J=7.2 Hz, 3H).

### Example 18

### 1-Methylethyl 2-{(3S)-3-[[(4,5-dimethyl-2-thienyl)methyl](ethyl)amino]-1-pyrrolidinyl}-3-pyridinecarboxylate

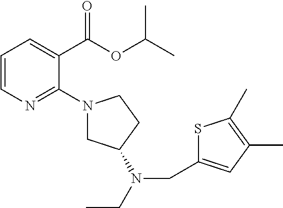 [SMILES: CCN(Cc1cc(C)c(C)s1)[C@H]1CCN(c2ncccc2C(=O)OC(C)C)C1]

A solution of 1-Methylethyl 2-[(3S)-3-(ethylamino)-1-pyrrolidinyl]-3-pyridinecarboxylate (125 mg, 0.45 mmol) and 4,5-dimethylthiophene-2-carbaldehyde (57 mg, 0.41 mmol) in THF (1 mL) was stirred at RT for 30 min. Then NaBH(OAc)₃ (287 mg, 1.35 mmol) was added. The mixture was stirred at room temperature overnight. The crude product was purified to obtain the title compound (60 mg, 33%) as yellow oil. LC-MS m/z 402 (M+H)⁺, 2.31 min (ret time); ¹H NMR (400 MHz, CDCl₃) δ 8.24-8.22 (m, 1H), 7.82-7.80 (m, 1H), 6.60-6.56 (m, 2H), 5.22-5.16 (m, 1H), 3.82 (s, 2H), 3.64-3.30 (m, 5H), 2.70-2.60 (m, 2H), 2.28 (s, 3H), 2.18-2.12 (m, 1H), 2.06 (s, 3H), 1.94-1.84 (m, 1H), 1.37-1.33 (dd, J=6.4 Hz, 6H), 1.07 (t, J=7.2 Hz, 3H).

### Example 19

### 1-Methylethyl 2-((3S)-3-{ethyl[(3-ethylphenyl)methyl]amino}-1-pyrrolidinyl)-3-pyridinecarboxylate

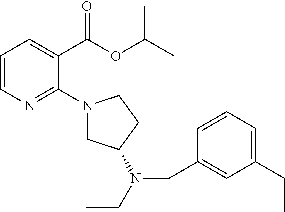 [SMILES: CCc1cccc(CN(CC)[C@H]2CCN(c3ncccc3C(=O)OC(C)C)C2)c1]

To a solution of 1-Methylethyl 2-[(3S)-3-(ethylamino)-1-pyrrolidinyl]-3-pyridinecarboxylate (400 mg, 1.44 mmol) and 3-ethylbenzaldehyde (176 mg, 1.31 mmol) in THF (1 mL) was added NaBH(OAc)₃ (835 mg, 3.94 mmol). The mixture was stirred at room temperature overnight. Water (15 mL) was added to the reaction mixture and it was extracted with ethyl acetate (3×15 mL). The combined organic layer was dried over Na₂SO₄ and concentrated to obtain the crude product. It was purified by Prep HPLC with a basic buffer condition to obtain the title compound (200 mg, 35%) as yellow oil. LC-MS m/z 396 (M+H)⁺, 2.38 min (ret time); ¹H NMR (400 MHz, CDCl₃) δ 8.24-8.23 (m, 1H), 7.82-7.80 (m, 1H), 7.25-7.14 (m, 3H), 7.07-7.05 (m, 1H), 6.60-6.57 (m, 1H), 5.21-5.14 (m, 1H), 2.70-3.36 (m, 7H), 2.66-2.60 (m, 4 H), 2.10-2.09 (m, 1H), 1.97-1.86 (m, 1H), 1.36-1.32 (m, 6H), 1.24-1.21 (m, 3H), 1.00 (t, J=6.8 Hz, 3H).

### Example 20

### 1-Methylethyl 2-((3S)-3-{ethyl[(4-ethylphenyl)methyl]amino}-1-pyrrolidinyl)-3-pyridinecarboxylate

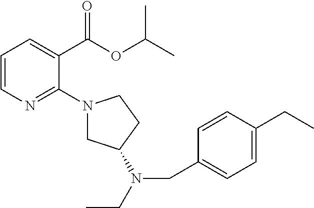 [SMILES: CCc1ccc(CN(CC)[C@H]2CCN(c3ncccc3C(=O)OC(C)C)C2)cc1]

To a solution of 1-Methylethyl 2-[(3S)-3-(ethylamino)-1-pyrrolidinyl]-3-pyridinecarboxylate in acetone (5 mL) was added 1-(bromomethyl)-4-ethylbenzene (119 mg, 0.6 mmol) and K₂CO₃ (123 mg, 0.9 mmol). The mixture was heated at 60° C. for 3 h. The reaction mixture was filtered and the filtrate was concentrated to obtain the crude product. It was purified by Prep HPLC with a basic buffer condition to obtain the title compound (80 mg, 34%) as yellow oil. LC-MS m/z 396 (M+H)⁺, 2.38 min (ret time); ¹H NMR (400 MHz, CDCl₃) δ 8.24-8.23 (m, 1H), 7.82-7.80 (m, 1H), 7.26-7.24 (m, 2H), 7.13-7.11 (m, 2H), 6.60-6.57 (m, 1H), 5.21-5.14 (m, 1H), 3.69-3.35 (m, 7 H), 2.65-2.60 (m, 4H), 2.13-2.09 (m, 1H), 2.08-1.85 (m, 1H), 1.36-1.32 (dd, J=6.4 Hz, 6H), 1.22 (t, J=7.6 Hz, 3H), 1.00 (t, J=7.2 Hz, 3H).

### Example 21

### 1-Methylethyl 2-((3S)-3-{ethyl[(2-ethylphenyl)methyl]amino}-1-pyrrolidinyl)-3-pyridinecarboxylate

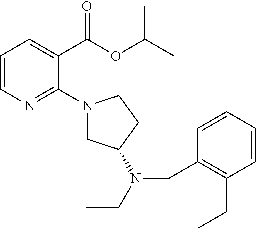 [SMILES: CCc1ccccc1CN(CC)[C@H]1CCN(c2ncccc2C(=O)OC(C)C)C1]

To a solution of 1-Methylethyl 2-[(3S)-(ethylamino)-1-pyrrolidinyl]-3-pyridinecarboxylate (139 mg, 0.5 mmol) in acetone (5 mL) was added 1-(bromomethyl)-2-ethylbenzene (100 mg, 0.5 mmol) and K₂CO₃ (104 mg, 0.75 mmol). The mixture was heated at 60° C. for 3 h. The reaction mixture was filtered and the filtrate was concentrated to obtain the crude product. It was purified by Prep HPLC with a basic buffer condition to obtain the title compound (69 mg, 35%) as yellow oil. LC-MS m/z 396 (M+H)⁺, 2.42 min (ret time); ¹H NMR (400 MHz, CDCl₃) δ 8.24-8.23 (m, 1H), 7.82-7.80 (m, 1H), 7.41-7.39 (m, 1H), 7.25-7.10 (m, 3H), 6.60-6.57 (m, 1H), 5.21-5.14 (m, 1H), 3.75-3.36 (m, 7H), 2.75-2.70 (m, 2H), 2.62-2.59 (m, 2H), 2.12-2.07 (m, 1H), 1.98-1.88 (m, 1H), 1.36-1.32 (dd, J=6.4 Hz, 6H), 1.20 (t, J=7.6 Hz, 3 H), 0.97 (t, J=6.8 Hz, 3H).

### Example 22

### 1-Methylethyl 2-(4-{[3-({[3-(methyloxy)phenyl]methyl}oxy)phenyl]methyl}-piperazinyl)-3-pyridinecarboxylate hydrochloride

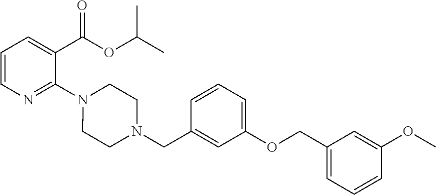 [SMILES: COc1cccc(COc2cccc(CN3CCN(c4ncccc4C(=O)OC(C)C)CC3)c2)c1]

To a solution of isopropyl 2-(4-(3-hydroxybenzyl)piperazin-1-yl)nicotinate (1.8 g, 5 mmol) and 1-(bromomethyl)-3-methoxybenzene (1.01 g, 5 mmol) in acetone (100 mL) was added K₂CO₃ (1.4 g, 10 mmol). The reaction mixture was refluxed overnight. It was filtered, concentrated to give the crude residue. It was purified by silica gel column chromatography eluting with 1:8 ratio ethyl acetate in petroleum ether to obtain colorless oil, which was stirred in 1M HCl in diethyl ether (10 mL) for 10 min. Solvent was removed to give the title compound (1 g, 43%) as a white solid. LC-MS m/z 476.3 (M+H)⁺, 1.27 min (ret time); ¹H NMR (400 MHz, DMSO-d₆) δ 1.29 (d, J=6 Hz, 6 H), 3.06-3.07 (m, 2H), 3.31-3.52 (m, 4H), 3.83 (d, J=14 Hz, 2H), 4.31 (d, J=4.4 Hz), 5.18-5.12 (m, 2H), 6.88-7.47 (m, 9H), 8.04-8.06 (m, 1H), 8.34-8.35 (m, 1H).

### Example 23

### 1-Methylethyl 2-{(3R)-3-[ethyl(phenylmethyl)amino]-1-pyrrolidinyl}-3-pyridinecarboxylate

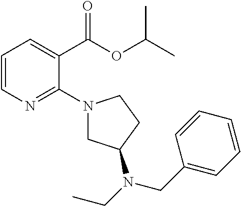 [SMILES: CCN(Cc1ccccc1)[C@@H]1CCN(c2ncccc2C(=O)OC(C)C)C1]

To a solution of (R)-isopropyl 2-(3-(tert-butoxycarbonyl(ethyl)amino)pyrrolidin-1-yl)nicotinate (480 mg, 1.27 mmol) in ethyl acetate (10 mL) at RT was introduced HCl (gas) for 30 min. Solvent was removed under reduced pressure. It was re-dissolved in acetone (20 mL). K₂CO₃ (351 mg) and (bromomethyl)benzene (435 mg, 2.54 mmol) were added. The resulting mixture was stirred at reflux for 16 h. Solid was filtered and the filtrate was concentrated to give the crude product. It was purified by silica gel column eluting with 1:10 ratio ethyl acetate in petroleum ether to give the desire target (350 mg, 75%) as pale yellow oil. LC-MS m/z 368.0 (M+H)⁺, 1.99 min (ret time)

### Example 24

### 2-{(3R)-3-[Ethyl(phenylmethyl)amino]-1-pyrrolidinyl}-3-pyridinecarboxylic acid

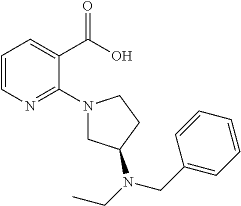 [SMILES: CCN(Cc1ccccc1)[C@@H]1CCN(c2ncccc2C(=O)O)C1]

To a solution of (R)-isopropyl 2-(3-(benzyl(ethyl)amino)pyrrolidin-1-yl)nicotinate (1.3 g, 3.5 mmol) in methanol (100 mL) was added NaOH (2.4 M/L, 50 mL). The mixture was heated at 100° C. overnight. Solvent was removed. The residue was washed with ethanol for three times to obtain the crude product. It was purified by silica gel column eluting with 1:10 ratio DCM in MeOH to obtain the title compound (1.15 g 100%) as yellow solid. LC-MS m/z 326(M+H)⁺, 1.28 min (ret time); ¹H NMR (400 MHz, DMSO-d₆) δ 11.80 (s, 1H), 8.27-8.25 (m, 1H), 8.09-8.06 (m, 1H), 7.74-7.73 (m, 2H), 7.46-7.45 (m, 3H), 6.91-6.88 (m, 1H), 4.57-3.44 (m, 7H), 3.07-2.945 (m, 2H), 2.57-2.51 (m, 1H), 2.50-2.39 (m, 1H), 1.27-1.22 (m, 3H).

### Example 25

### 1-Methylethyl 2-{methyl[(3R)-3-pyrrolidinyl]amino}-3-pyridinecarboxylate hydrochloride

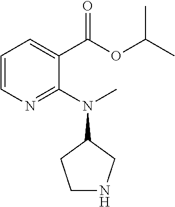

To a solution of isopropyl (R)-isopropyl 2-{[1-(tert-butoxycarbonyl)pyrrolidin-3-yl](methyl)amino}nicotinate (3.2 g, 8.8 mmol) in methanol (5 mL) at 0° C. was added acetyl chloride (1.4 g, 17.6 mmol) dropwise. The mixture was stirred at room temperature for 30 min. Solvent was evaporated to dryness under reduced pressure. To the residue was added EtOAc (50 mL), the mixture was stirred at 0° C. for 30 min. The white solid was collected via filtration, washed with EtOAc (2×5 mL), and dried in vacuum to give the title compound (2.4 g, 90%) as white solid. LC-MS m/z 264.2 (M+H)⁺, 0.94 min (ret time); ¹H NMR (400 MHz, DMSO-d₆) δ 1.32 (d, J=3.2 Hz, 6H), 2.21-2.04 (m, 4H), 2.78 (s, 3H), 3.53-3.13 (m, 4H), 5.12-4.90 (m, 2H), 6.87-6.84 (m, 1H), 7.93-7.91 (m, 1H), 8.30-8.28 (m, 1H), 9.21 (d, J=10.2 Hz, 2H).

### Example 26

### 1-methylethyl2-(4-{[3-(Phenylmethyl)phenyl]methyl}-1-piperazinyl)-3-pyridine carboxylate hydrochloride

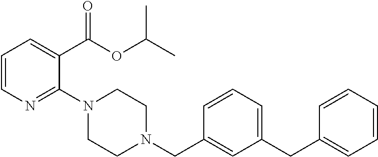 [SMILES: CC(C)OC(=O)c1cccnc1N1CCN(Cc2cccc(Cc3ccccc3)c2)CC1]

To a solution of 1-methylethyl 2-(1-piperazinyl)-3-pyridinecarboxylate hydrochloride (100 mg, 0.35 mmol) in EtOAc (10 mL) was added Et₃N (53 mg, 0.52 mmol), the mixture was stirred at room temperature for 30 min and filtered. The filtrate was concentrated to dryness to give the free amine. The above residue was dissolved in DCE (20 mL), 3-benzylbenzaldehyde (69 mg, 0.35 mmol) and HOAc (two drops) was added, the mixture was stirred at room temperature for 20 min. NaBH(OAc)₃ (148 mg, 0.7 mmol) was added in one portion, the resulting mixture was stirred at room temperature for 16 h. 10% NaOH (aq. 10 mL) was added, the mixture was extracted with DCM (3×150 mL). The combined organics were washed with brine (2×30 mL), dried over MgSO₄, filtered, concentrated to give the crude product which was purified via chromatography on silica gel (200-300 um, 10 g, elution with 25% EtOAc of hexane) to afford the free base of the title compound (120 mg, 81%) as yellow oil. To the free base of the title compound (120 mg, 0.28 mmol) in diethyl ether (2 mL) was added HCl (1 M in diethyl ether, 3 mL). Solid was collected via filtration, dried in vacuo to give the title compound (130 mg) as white solid. LC-MS m/z 430.1 (M+H)⁺, 2.28 min (ret time); ¹H NMR (400 MHz, DMSO-d₆) δ 11.81 (s, br, 1H), 8.35 (d, J=3.2 Hz, 1H), 8.07 (d, J=7.2 Hz, 1H), 7.57-6.98 (m, 10H), 5.11-5.08 (m, 1H), 4.33 (d, J=3.6 Hz, 2H), 3.96 (s, 2H), 3.85-3.04 (m, 8H), 1.29 (d, J=6.4 Hz, 6H).

### Example 27

### 1-Methylethyl 2-(4-{[4-(phenylmethyl)phenyl]methyl}-1-piperazinyl)-3 pyridinecarboxylate hydrochloride

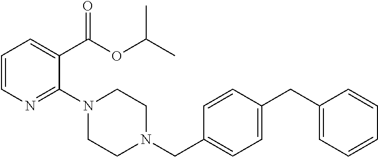 [SMILES: CC(C)OC(=O)c1cccnc1N1CCN(Cc2ccc(Cc3ccccc3)cc2)CC1]

Following the general procedure of 1-methylethyl 2-(4-{[3-(Phenylmethyl)phenyl]methyl}-1-piperazinyl)-3-pyridinecarboxylate hydrochloride 1-methylethyl 2-(1-piperazinyl)-3-pyridinecarboxylate hydrochloride (100 mg, 0.35 mmol) was stirred in EtOAc (10 mL) with Et₃N (53 mg, 0.52 mmol) to give a free amine, which was then reacted with 4-benzylbenzaldehyde (69 mg, 0.35 mmol), HOAC (two drops) and NaBH(OAc)₃ (148 mg, 0.70 mmol) to give the free base of the title compound (91 mg, 61%) as colorless oil. It was stirred in HCl (1M in diethyl ether, 1 mL) to give the title compound (54 mg) as white solid. LC-MS m/z 430.1 (M+H)⁺, 2.29 min (ret time); ¹H NMR (400 MHz, DMSO-d₆) δ 11.77 (s, br, 1H), 8.34 (s, 1H), 8.06 (d, J=6.8 Hz, 1H), 7.59-6.99 (m, 10H), 5.11-5.08 (m, 1H), 4.30 (s, 2H), 3.96 (s, 2H), 3.84-3.03 (m, 8 H), 1.28 (d, J=5.6 Hz, 6H).

### Example 28

### 1-Methylethyl 2-[4-(2-phenylethyl)-1-piperazinyl]-3-pyridinecarboxylate hydrochloride

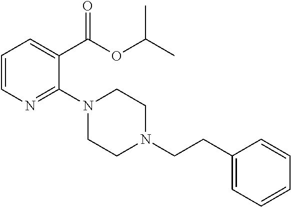 [SMILES: CC(C)OC(=O)c1cccnc1N1CCN(CCc2ccccc2)CC1]

A mixture of 1-methylethyl 2-(1-piperazinyl)-3-pyridinecarboxylate (200 mg, 0.8 mmol) and 2-phenylacetaldehyde (96.4 mg, 0.8 mmol) in THF (20 mL) was stirred at RT for 10 min. NaBH (OAc)₃ (340 mg, 1.6 mmol) was added. The reaction mixture was stirred at RT overnight. Water (10 mL) was added. The water layer was extracted with ethyl acetate. The organic layer was washed with brine, dried and concentrated. The residue was purified by Prep HPLC to obtain pale yellow oil, which was stirred in 1 M HCl in diethyl ether for 10 min. Solvent was removed to give the desired product (62 mg, 22%) as pale yellow solid. LC-MS m/z 354.1 (M+H)⁺, 2.06 min (ret time); ¹H NMR (400 MHz, CDCl₃) δ 1.40-1.49 (m, 6H), 3.33 (s, 4H), 3.46-4.45 (m, 8H), 5.28 (s, 1H), 7.28-7.31 (d, 8H), 8.46 (s, 2H), 13.54 (s, 1H).

### Example 29

### 1-Methylethyl2-(4-{[4-(3-phenylpropyl)phenyl]methyl}-1-piperazinyl)-3-pyridine carboxylate hydrochloride

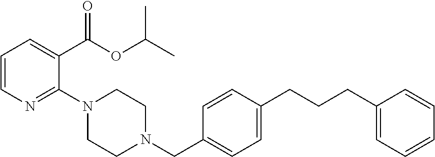 [SMILES: CC(C)OC(=O)c1cccnc1N1CCN(Cc2ccc(CCCc3ccccc3)cc2)CC1]

Following the general procedure of 1-methylethyl 2-(4-{[3-(Phenyl methyl)phenyl]methyl}-1-piperazinyl)-3-pyridinecarboxylate hydrochloride 1-Methylethyl 2-(1-piperazinyl)-3-pyridinecarboxylate hydrochloride (100 mg, 0.35 mmol) was stirred in THF (10 mL) with Et₃N (53 mg, 0.52 mmol) to give a free amine, which was then reacted with 4-(3-phenylpropyl)benzaldehyde (79 mg, 0.35 mmol), HOAC (two drops) and NaBH(OAc)₃ (148 mg, 0.70 mmol) to give the free base of the title compound (100 mg, 63%) as colorless oil. It was stirred in HCl (1M in diethyl ether, 1 mL) to give the title compound (140 mg) as white solid. LC-MS m/z 458.1 (M+H)⁺, 2.43 min (ret time); ¹H NMR (400 MHz, DMSO-d₆) δ 11.90 (s, br, 1H), 9.19 (s, br, 2H), 8.34 (d, J=3.2 Hz, 1H), 8.08-8.06 (m, 1H), 7.59 (d, J=7.6 Hz, 2H), 7.28-6.98 (m, 8H), 5.11-5.08 (m, 1H), 4.32 (d, J=4.0 Hz, 2H), 3.83 (d, J=13.6 Hz, 2H), 3.56-3.53 (m, 2H), 3.53-3.36 (m, 2H), 3.07-3.06 (m, 2H), 2.64-2.58 (m, 4H), 1.97-1.87 (m, 2H), 1.28 (d, J=6.0 Hz, 6H).

### Example 30

### 1-Methylethyl 2-[4-({3-[methyl(phenylcarbonyl)amino]phenyl}methyl)-1-piperazinyl]-3-pyridinecarboxylate hydrochloride

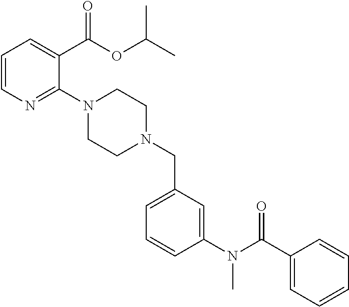 [SMILES: CC(C)OC(=O)c1cccnc1N1CCN(Cc2cccc(N(C)C(=O)c3ccccc3)c2)CC1]

To a solution of 1-methylethyl 2-[4-({3-[(phenylcarbonyl)amino]phenyl}methyl)-1-piperazinyl]-3-pyridinecarboxylate (200 mg, 0.52 mmol) in DMF (15 mL) was added sodium hydride (60%, 20.8 mg, 0.52 mmol). The reaction mixture was stirred at RT for 10 min, and then was added iodomethane (80 mg, 0.53 mmol). The reaction mixture was stirred overnight. Water (40 mL) was added, extracted with ethyl acetate. The organic layer was washed with water and brine, dried with anhydrous sodium sulfate, filtered and concentrated to give the crude residue. It was purified by Prep TLC (DCM: methanol=20: 1) to the free base of the title compound (40 mg, 16.3%) as colorless oil. The oil was dissolved in diethyl ether (5 mL). 1M HCl in diethyl ether was added and stirred for 10 min. Solvent was removed to give the title compound as yellow solid (40 mg, 98%). LC-MS m/z 473.1 (M+H)⁺, 1.95 min (ret time); ¹H NMR (400 MHz, CDCl₃) δ 1.06-1.35 (m, 10H), 3.11 (s, 2H), 3.45 (d, 3H), 4.11 (s, 4H), 5.21 (s, 1H), 7.10-7.52 (m, 12H), 13.12 (s, 1H).

### Example 31

### 1-Methylethyl 2-[4-({4-[methyl(phenylcarbonyl)amino]phenyl}methyl)-1-piperazinyl]-3-pyridinecarboxylate hydrochloride

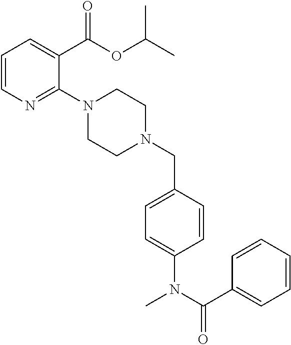 [SMILES: CC(C)OC(=O)c1cccnc1N1CCN(Cc2ccc(N(C)C(=O)c3ccccc3)cc2)CC1]

The procedure was similar with 1-Methylethyl 2-[4-({3-[methyl(phenylcarbonyl)amino]phenyl}methyl)-1-piperazinyl]-3-pyridinecarboxylate hydrochloride. 1-Methylethyl 2-[4-({4-[(phenylcarbonyl)amino]phenyl}methyl)-1-piperazinyl]-3-pyridine carboxylate (100 mg, 0.26 mmol), iodomethane (37 mg, 0.26 mmol), sodium hydride (60%, 11 mg, 0.26 mmol) and 1 M diethyl ether were reacted to give the desired product (40 mg, 28%) as white solid. LC-MS m/z 473.2 (M+H)⁺, 1.19 min (ret time); ¹H NMR (400 MHz, Methanol-d₄) δ 3.31-3.33 (m, 6H), 3.40-3.68 (m, 9H), 3.98 (s, 2H), 4.44 (s, 2H), 5.25-5.28 (t, 1H), 7.23-7.33 (m, 8H), 7.55-7.57 (d, 2H), 8.38-8.38 (d, 1H), 8.58-8.60 (d, 1H).

### Example 32

### 1-methylethyl 2-[4-({3-[(dimethylamino)carbonyl]phenyl}methyl)-1-piperazinyl]-3-pyridinecarboxylate hydrochloride

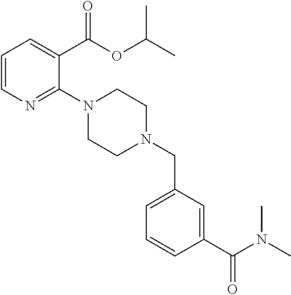 [SMILES: CC(C)OC(=O)c1cccnc1N1CCN(Cc2cccc(C(=O)N(C)C)c2)CC1]

The procedure was similar with 1-Methylethyl 2-((3S)-3-{ethyl[(4-ethylphenyl)methyl]amino}-1-pyrrolidinyl)-3-pyridinecarboxylate. 3-{[4-(3-{[(1-Methylethyl)oxy]carbonyl}-2-pyridinyl)-1-piperazinyl]methyl}benzoic acid (300 mg, 0.78 mmol), dimethylamine hydrochloride (63.8 mg, 0.78 mmol), EDCI (150 mg, 0.78 mmol), HOBt (105 mg, 0.78 mmol) and TEA (157.8 mg, 1.56 mmol) were reacted to give the free base of the title compound (60 mg, 18.7%) as colorless oil. The oil was dissolved in diethyl ether (10 mL). 1M HCl in diethyl ether was added and stirred for 10 min. Solvent was removed to give the title compound as white solid (60 mg). LC-MS m/z 411.1 (M+H)⁺, 1.79 min (ret time); ¹H NMR (400 MHz, DMSO-d₆&D₂O) δ 1.25-1.26 (d, 6H), 2.89-3.42 (m, 12H), 3.80 (s, 2H), 4.39 (s, 2H), 5.03-5.09 (m, 1H), 6.98-7.01 (m, 1H), 7.47-7.62 (m, 4H), 8.03-8.05 (d, 1H), 8.30-8.31 (d, 1H).

### Example 33

### 1-Methylethyl 2-[4-({4-[(dimethylamino)carbonyl]phenyl}methyl)-1-piperazinyl]-3-pyridinecarboxylate hydrochloride

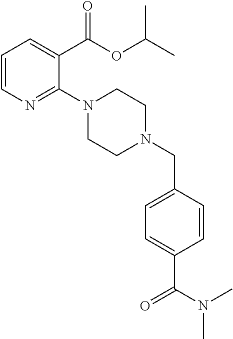 [SMILES: CC(C)OC(=O)c1cccnc1N1CCN(Cc2ccc(C(=O)N(C)C)cc2)CC1]

The procedure was similar with 1-Methylethyl 2-((3S)-3-{ethyl[(4-ethylphenyl)methyl]amino}-1-pyrrolidinyl)-3-pyridinecarboxylate 4-{[4-(3-{[(1-Methylethyl)oxy]carbonyl}-2-pyridinyl)-1-piperazinyl]methyl}benzoic acid (300 mg, 0.78 mmol), dimethylamine hydrochloride (63.8 mg, 0.78 mmol), EDCI (150 mg, 0.78 mmol), HOBt (105 mg, 0.78 mmol) and TEA (157.8 mg, 1.56 mmol) were reacted to give the title compound (60 mg, 18.7%) as colorless oil. The oil was dissolved in diethyl ether (10 mL), and to the solution was added 1M HCl in diethyl ether, stirred for 10 min. Solvent was removed to give the title compound as white solid (60 mg). LC-MS m/z 411.1 (M+H)⁺, 1.78 min (ret time); ¹H NMR (400 MHz, CDCl₃) δ 1.41 (s, 6H), 3.13 (s, 6H), 3.47-3.75 (m, 4H), 4.10-4.43 (m, 6H), 5.26 (s, 1H), 7.54 (s, 3H), 7.83 (s, 2H), 8.46 (s, 2H), 13.25 (s, 1H).

### Example 34

### 1-Methylethyl2-(4-{[4-(hydroxymethyl)phenyl]methyl}-1-piperazinyl)-3-pyridine carboxylate hydrochloride

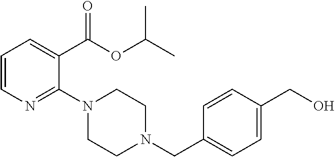 [SMILES: CC(C)OC(=O)c1cccnc1N1CCN(Cc2ccc(CO)cc2)CC1]

The solution of 1-methylethyl 2-{4-[(4-formylphenyl)methyl]-1-piperazinyl}-3-pyridinecarboxylate (4.53 g, 0.012 mmol) in methanol (50 mL) at 0° C. was added NaBH₄ (560 mg, 0.015 mmol). The reaction mixture was stirred overnight. Solvent was removed under reduce pressure. It was dissolved in THF, washed with brine, dried over Na₂SO₄ and concentrated to give the free base of the title compound (4 g, 88%) as yellow oil. It was dissolved in ether (10 mL); the solution of HCl in ether (5 mL) was added. It was stirred at room temperature for 10 min. Solvent was removed to give the title compound (3.5 g, 70%) as white solid. LC-MS m/z 370.2 (M+H)⁺, 1.73 min (ret time); ¹H NMR (400 MHz, CDCl₃) 1.38-1.39 (d, J=4.4 Hz, 6H) 3.41 (s, 4H) 4.03 (s, 2H) 4.28 (s, 4H) 4.70 (s, 2H) 5.23 (s, 1H) 7.17 (s, 1H) 7.41 (s, 2H) 7.63 (s, 2 H) 8.37 (s, 2H) 13.03 (s, 1H)

### Example 35

### 1-Methylethyl 2-(4-{[3-(phenylthio)phenyl]methyl}-1-piperazinyl)-3-pyridinecarboxylate hydrochloride

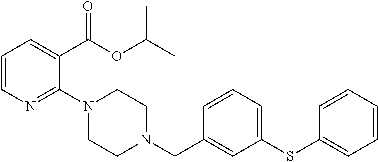 [SMILES: CC(C)OC(=O)c1cccnc1N1CCN(Cc2cccc(Sc3ccccc3)c2)CC1]

1-Methylethyl 2-(1-piperazinyl)-3-pyridinecarboxylate hydrochloride (200 mg, 0.70 mmol) was stirred in EtOAc (10 mL) with Et₃N (106 mg, 1.05 mmol) to give a free amine, which was then reacted with 3-(phenylthio)benzaldehyde (214 mg, 1 mmol), HOAC (two drops) and NaBH(OAc)₃ (297 mg, 1.40 mmol) to give the free base of the title compound (36 mg, 11.5%) as yellow oil. It was stirred in HCl (1 M in diethyl ether, 1 mL) to give the title compound (35 mg) as white solid. LC-MS m/z 448.1 (M+H)⁺, 1.22 min (ret time); ¹H NMR (400 MHz, MEOD) δ 8.64 (d, J=4.8 Hz, 1H), 8.39 (s, 1H), 7.60-7.36 (m, 10H), 5.29 (s, 1H), 4.47 (s, 2H), 3.99-3.45 (m, 8H), 1.42 (d, J=4.8 Hz, 6H).

### Example 36

### 1-Methylethyl 2-(4-{[4-(phenylthio)phenyl]methyl}-1-piperazinyl)-3-pyridinecarboxylate hydrochloride

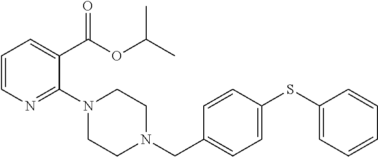 [SMILES: CC(C)OC(=O)c1cccnc1N1CCN(Cc2ccc(Sc3ccccc3)cc2)CC1]

Following the general procedure of 1-methylethyl 2-(4-{[3-(Phenylmethyl)phenyl]methyl}-1-piperazinyl)-3-pyridinecarboxylate hydrochloride 1-Methylethyl 2-(1-piperazinyl)-3-pyridinecarboxylate hydrochloride (200 mg, 0.70 mmol) was stirred in EtOAc (20 mL) with Et₃N (106 mg, 1.05 mmol) to give a free amine, which was then reacted with 4-(phenylthio)benzaldehyde (150 mg, 0.7 mmol), HOAC (four drops) and NaBH(OAc)₃ (297 mg, 1.40 mmol) to give the free base of the title compound (110 mg, 35%) as yellow oil. It was stirred in HCl (1M in diethyl ether, 2 mL) to give the title compound (30 mg) as yellow solid. LC-MS m/z 448.1 (M+H)±, 1.40 min (ret time); ¹H NMR (400 MHz, MEOD) δ 8.54 (d, J=5.6 Hz, 1H), 8.38 (s, 1 H), 7.55-7.28 (m, 10H), 5.27 (s, 1H), 4.44 (s, 2H), 3.98 (s, 2H), 3.61-3.42 (m, 6H), 1.41 (d, J=5.2 Hz, 6H).

### Example 37

### 1-Methylethyl 2-[4-({3-[(phenylmethyl)thio]phenyl}methyl)-1-piperazinyl]-3-pyridine carboxylate hydrochloride

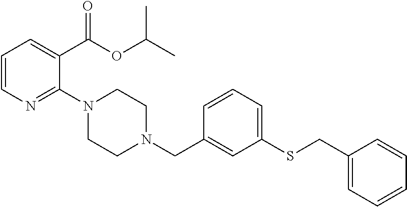 [SMILES: CC(C)OC(=O)c1cccnc1N1CCN(Cc2cccc(SCc3ccccc3)c2)CC1]

Following the general procedure of 1-methylethyl 2-(4-{[3-(Phenylmethyl)phenyl]methyl}-1-piperazinyl)-3-pyridinecarboxylate hydrochloride 1-Methylethyl 2-(1-piperazinyl)-3-pyridinecarboxylate hydrochloride (200 mg, 0.70 mmol) was stirred in EtOAc (20 mL) with Et₃N (106 mg, 1.05 mmol) to give a free amine, which was then reacted with 3-(benzylthio)benzaldehyde (192 mg, 0.84 mmol), HOAC (two drops) and NaBH(OAc)₃ (297 mg, 1.40 mmol) to give the free base of the title compound (242 mg, 75%) as pale yellow oil. It (50 mg) was stirred in HCl (1 M in diethyl ether, 2 mL) to give the title compound (53 mg) as white solid. LC-MS m/z 462.1 (M+H)⁺, 1.42 min (ret time); ¹H NMR (400 MHz, MEOD) δ 8.70 (d, J=6.8 Hz, 1H), 8.40 (d, J=4.0 Hz, 1H), 7.65 (s, 1H), 7.48-7.22 (m, 9H), 5.30-5.28 (m, 1H), 4.54 (s, 2H), 4.28 (s, 2H), 3.99-3.32 (m, 8H), 1.43 (d, J=6.0 Hz, 6H).

### Example 38

### 1-Methylethyl 2-[4-({4-[(phenylmethyl)thio]phenyl}methyl)-1-piperazinyl]-3-pyridine carboxylate hydrochloride

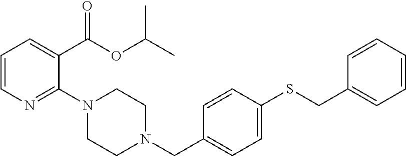 [SMILES: CC(C)OC(=O)c1cccnc1N1CCN(Cc2ccc(SCc3ccccc3)cc2)CC1]

To a solution of isopropyl 2-[4-(4-mercaptobenzyl)piperazin-1-yl]nicotinate (100 mg, 0.27 mmol) in acetone (20 mL) was added K₂CO₃ (56 mg, 0.4 mmol) followed by BnBr (51 mg, 0.3 mmol). The resulting mixture was stirred at room temperature for 30 min. The mixture was filtered. The filtrate was concentrated to dryness. The residue was purified by flash chromatography eluting with 0 to 25% EtOAc in hexane to give the free base of the title compound (65 mg, 52%) as pale yellow oil. It (30 mg, 0.065 mmol) was dissolved in diethyl ether (1 mL), HCl (1 M in diethyl ether, 1 mL) was added. The mixture was stirred at room temperature for 5 min. Solvent was evaporated to dryness to give the title compound (32 mg) as white solid. LC-MS m/z 462.1 (M+H)⁺, 1.42 min (ret time); ¹H NMR (400 MHz, MEOD) δ 8.68 (s, 1H), 8.39 (s, 1H), 7.57-7.22 (m, 10H), 5.29 (s, 1H), 4.46 (s, 2H), 4.23 (s, 2H), 4.01-3.47 (m, 8H), 1.43 (s, 6H).

### Example 39

### 1-Methylethyl 2-[(3S)-3-(ethylamino)-1-pyrrolidinyl]-3-pyridinecarboxylate hydrochloride

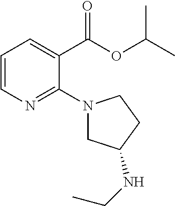 [SMILES: CCN[C@H]1CCN(c2ncccc2C(=O)OC(C)C)C1]

To a solution of isopropyl(S)-isopropyl2-(3-(tert-butoxycarbonyl(ethyl)amino) pyrrolidin-1-yl)nicotinate (4.3 g, 11.4 mmol) in ethyl ether (30 mL) was added HCl in ethyl ether (1M, 30 mL). The reaction mixture was stirred at RT for 30 min, and then evaporated to give the crude product. It was washed with ethyl ether. Solid was collected to give the title compound (4.0 g, 100%). LC-MS m/z 278.3 (M+H)⁺, 0.95 min (ret time); ¹H NMR (400 MHz, DMSO-d₆) δ 1.24 (t, 3H), 1.35 (t, 6H), 2.28-2.37 (m, 2H), 2.50 (s, 2H), 2.90-3.02 (m, 2H), 3.16 (s, 1H), 3.58-3.72 (m, 4H), 3.86 (m, 1H), 5.10 (m, 1H), 6.92 (m, 1H), 8.08 (d, 1H), 8.26 (d, 1H), 9.56 (s, 1H), 9.73 (s, 1H).

### Example 40

### 1-Methylethyl2-(4-{[4-(2-phenylethyl)phenyl]methyl}-1-piperazinyl)-3-pyridinecarboxylate hydrochloride

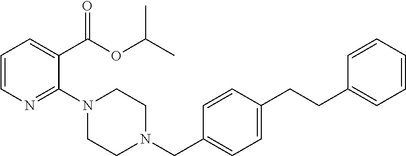 [SMILES: CC(C)OC(=O)c1cccnc1N1CCN(Cc2ccc(CCc3ccccc3)cc2)CC1]

To a solution of 1-methylethyl 2-(1-piperazinyl)-3-pyridinecarboxylate (100 mg, 0.4 mmol) and 4-phenethylbenzaldehyde (84 mg, 0.4 mmol) in DCE (5 mL) was added HOAc (36 mg, 0.6 mmol) followed by NaBH(OAc) (3 9170 mg, 0.8 mmol). The resulting mixture was stirred at RT for 16 h. 10% NaOH (aq, 10 mL) was added, the mixture was extracted with DCM (3×50 mL). The combined organic layer was washed with brine (2×30 mL), dried over MgSO₄, filtered, and concentrated to give the crude product which was purified by pre-TLC (eluting with 15% EtOAc in n-Hexane) to give the free base of the title compound (28 mg). It was dissolved in Et₂O (1 mL), HCl/Et₂O (1M, 1 mL, 1 mmol) was added. The mixture was stirred at RT for 10 min. The solid was collected via filtration, dried in vacuo to give the title compound (27 mg, 14%). LC-MS m/z 444.1 (M+H)⁺, 1.40 min (ret time); ¹H NMR (400 MHz, DMSO-d₆) δ 1.30 (s, 6H), 2.50 (s, 3H), 2.89 (s, 4H), 3.00-3.08 (m, 2H), 3.34 (d, 2H), 3.48 (t, 2H), 3.82 (d, 2H), 4.31 (d, 1H), 5.10 (m, 1H), 6.98 (m, 1H), 7.16-7.30 (m, 7 H), 7.55 (m, 2H), 8.04 (dd, 1H), 8.34 (dd, 1H), 11.64 (s, 1H).

### Example 41

### 1-Methylethyl 2-(4-{[3-(hydroxymethyl)phenyl]methyl}-1-piperazinyl)-3-pyridine carboxylate

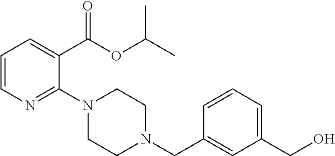 [SMILES: CC(C)OC(=O)c1cccnc1N1CCN(Cc2cccc(CO)c2)CC1]

To the solution of isopropyl 2-(piperazin-1-yl)nicotinate (3.88 g, 15.5 mmol) and 3-(hydroxymethyl)benzaldehyde (2.11 g, 15.5 mmol) in THF (10 mL) was added NaBH(AcO)₃ (9.9 g, 46.5 mmol). The reaction mixture was stirred at room temperature overnight. Water (50 mL) was added and it was extracted with ethyl acetate (3×50 mL). The organic layer was dried over Na₂SO₄ and concentrated to give the crude product. It was purified by silica gel column eluting with 2% of methanol and 1% of Et₃N in CH₂Cl₂ to give the title compound (3 g, 52%) as yellow solid. LC-MS m/z 370.1 (M+H)⁺, 1.08 min (ret time); ¹H NMR (400 MHz, CDCl₃) δ 1.33-1.34 (d, J=6.0 Hz, 6H) 2.02 (s, 1H) 2.63 (s, 4H) 3.46-3.49 (m, 4H) 3.62 (s, 2H) 4.70 (s, 2H) 5.15-5.21 (m, 1H) 6.71-6.74 (m, 1H) 7.26-7.39 (m, 4H) 7.92-7.95 (m, 1 H) 8.24-8.26 (m, 1H)

### Example 42

### 1-Methylethyl2-[(3R)-3-(ethyl{[4-({ethyl[(3R)-1-(2-methylpropanoyl)-3-pyrrolidinyl]amino}methyl)phenyl}methyl]amino)-1-pyrrolidinyl]-3-pyridinecarboxylate

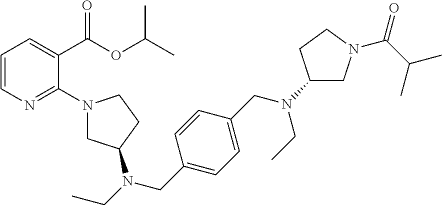 [SMILES: CCN(Cc1ccc(CN(CC)[C@@H]2CCN(c3ncccc3C(=O)OC(C)C)C2)cc1)[C@@H]1CCN(C(=O)C(C)C)C1]

To a solution of 1-methylethyl 2-[(3R)-3-(ethylamino)-1-pyrrolidinyl]-3-pyridinecarboxylate (85 mg, 0.385 mmol) and potassium carbonate (160 mg, 1.155 mmol) in acetone (10 mL) at room temperature was added (3R)—N-{[4-(bromomethyl)phenyl]methyl}-N-ethyl-1-(2-methylpropanoyl)-3-pyrrolidinamine (230 mg, 0.385 mmol) in one portion. The resulting mixture was heated to reflux for 24 h. It was cooled to room temperature. The reaction mixture was filtered and the filtrate was concentrated under reduced pressure to give crude product. It was purified by Pre-TLC eluting with EtOAc to give the title compound (26 mg, 11%) as pale yellow solid. LC-MS m/z 564.4 (M+H)⁺, 2.21 min (ret time); ¹H NMR (400 MHz, CDCl₃) δ 8.26-8.24 (m, 1H), 7.84-7.82 (m, 1H), 7.83-7.28 (m, 4H), 6.65-6,7-(m, 1H), 5.20-5.17 (m, 1H), 3.70-3.28 (m, 14H), 2.66-2.59 (m, 5H), 2.10-1.90 (m, 4H), 1.38-1.34 (m, 6H), 1.14-0.99 (m, 12H).

### Example 43

### 1-methylethyl 2-{(3R)-3-[(3-biphenylylmethyl)(ethyl)amino]-1-pyrrolidinyl}-3-pyridine carboxylate

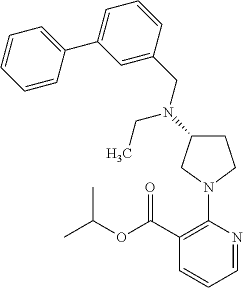 [SMILES: CCN(Cc1cccc(-c2ccccc2)c1)[C@@H]1CCN(c2ncccc2C(=O)OC(C)C)C1]

In an A-vial, acetaldehyde (5.30 mg, 0.120 mmol) and 1-methylethyl 2-[(3R)-3-amino-1-pyrrolidinyl]-3-pyridinecarboxylate (30.0 mg, 0.120 mmol) were added to the solution of dimethyl sulfoxide (DMSO) (1.5 ml) with acetic acid (7.23 mg, 0.120 mmol). The solution was stirred for 1 h at room temperature. Then MP-B(OAc)₃H (282 mg, 1.203 mmol) was added. The resulted mixture was stirred at room temperature for 12 hours after which time sodium cyanoborohydride (76 mg, 1.203 mmol) was added and the contents were stirred for another 12 h. To the resulting mixture 3-biphenyl benzylaldehyde (37.9 mg, 0.361 mmol) was added and the solution was stirred for 3 hr. The polymer was filtered and the crude product was dissolved in DMSO, and purified on a Gilson HPLC (XBridge 19×100 mm 5 preparatory column), eluting with acetonitrile and 0.1% aqueous NH₄OH. The desired fractions were concentrated under a stream of nitrogen at 50° C., giving 7.67 mg (%) of the title compound. LC-MS m/z 444.4 (M+H)⁺, 1.05 min (ret time).

Following the procedure as described above in the preparation of 1-methylethyl 2-{(3R)-3-[(3-biphenylylmethyl)(ethyl)amino]-1-pyrrolidinyl}-3-pyridinecarboxylate, 1-methylethyl 2-[(3R)-3-amino-1-pyrrolidinyl]-3-pyridinecarboxylate (30.0 mg, 0.120 mmol) was reacted with the appropriate aldehyde to yield the examples listed in Table I.

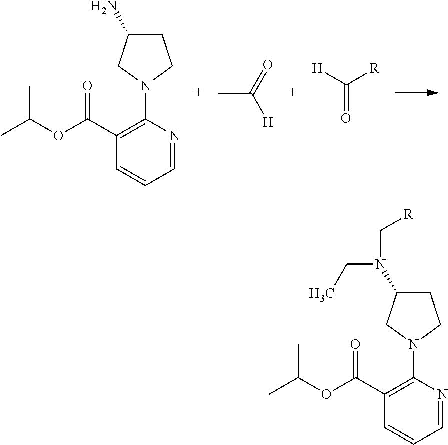

TABLE I

| | | | LC-MS | |
|---|---|---|---|---|
| | Aldehyde | | m/z | RT |
| Example | or Ketone | Product | (M + H)⁺ | (min) |
| Example 44 | 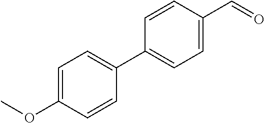 [SMILES: COc1ccc(-c2ccc(C=O)cc2)cc1] | 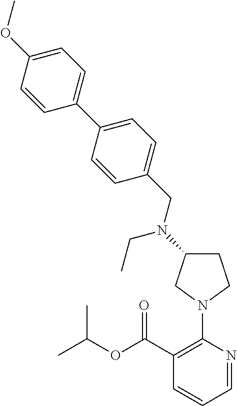 [SMILES: CCN(Cc1ccc(-c2ccc(OC)cc2)cc1)[C@@H]1CCN(c2ncccc2C(=O)OC(C)C)C1] | 474.3 | 1.0 |
| | | 1-methylethyl 2-[(3R)-3- | | |
| | | (ethyl{[4′-(methyloxy)-4- | | |
| | | biphenylyl]methyl}amino)- | | |
| | | 1-pyrrolidinyl]-3- | | |
| | | pyridinecarboxylate | | |
| Example 45 | 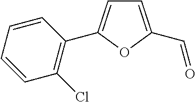 [SMILES: O=Cc1ccc(-c2ccccc2Cl)o1] | 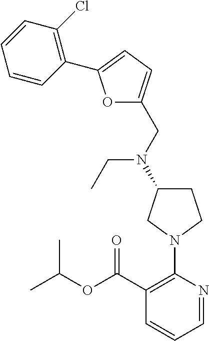 [SMILES: CCN(Cc1ccc(-c2ccccc2Cl)o1)[C@@H]1CCN(c2ncccc2C(=O)OC(C)C)C1] | 468.3 | 1.1 |
| | | 1-methylethyl 2-{(3R)-3- | | |
| | | [{[5-(2-chlorophenyl)-2- | | |
| | | furanyl]methyl}(ethyl)amino]- | | |
| | | 1-pyrrolidinyl}-3- | | |
| | | pyridinecarboxylate | | |
| Example 46 | 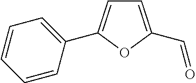 [SMILES: O=Cc1ccc(-c2ccccc2)o1] | 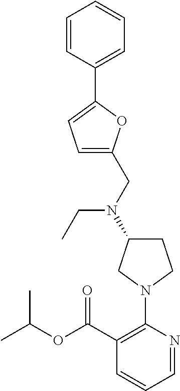 [SMILES: CCN(Cc1ccc(-c2ccccc2)o1)[C@@H]1CCN(c2ncccc2C(=O)OC(C)C)C1] | 435.2 | 0.8 |
| | | 1-methylethyl 2-((3R)-3- | | |
| | | {ethyl[(5-phenyl-2- | | |
| | | furanyl)methyl]amino}-1- | | |
| | | pyrrolidinyl)-3- | | |
| | | pyridinecarboxylate | | |
| Example 47 | 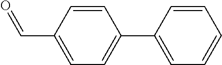 [SMILES: O=Cc1ccc(-c2ccccc2)cc1] | 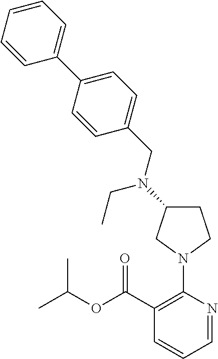 [SMILES: CCN(Cc1ccc(-c2ccccc2)cc1)[C@@H]1CCN(c2ncccc2C(=O)OC(C)C)C1] | 444.4 | 1.0 |
| | | 1-methylethyl 2-{(3R)-3- | | |
| | | [(4-biphenylyl methyl) | | |
| | | (ethyl)amino]-1- | | |
| | | pyrrolidinyl}-3-pyridine | | |
| | | carboxylate | | |
| Example 48 | 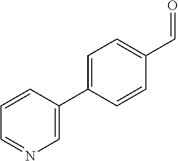 [SMILES: O=Cc1ccc(-c2cccnc2)cc1] | 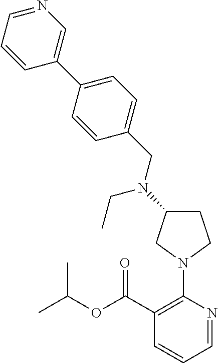 [SMILES: CCN(Cc1ccc(-c2cccnc2)cc1)[C@@H]1CCN(c2ncccc2C(=O)OC(C)C)C1] | 445.5 | 0.8 |
| | | 1-methylethyl 2-[(3R)-3- | | |
| | | (ethyl{[4-(3-pyridinyl) | | |
| | | phenyl]methyl}amino)-1- | | |
| | | pyrrolidinyl]-3-pyridine | | |
| | | carboxylate | | |
| Example 49 | 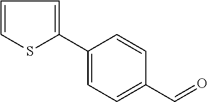 [SMILES: O=Cc1ccc(-c2cccs2)cc1] | 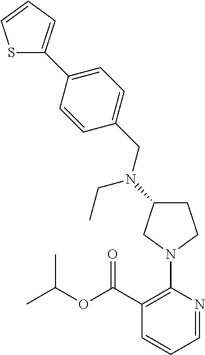 [SMILES: CCN(Cc1ccc(-c2cccs2)cc1)[C@@H]1CCN(c2ncccc2C(=O)OC(C)C)C1] | 450.2 | 1.0 |
| | | 1-methylethyl 2-[(3R)-3- | | |
| | | (ethyl{[4-(2-thienyl)phenyl] | | |
| | | methyl}amino)-1- | | |
| | | pyrrolidinyl]-3-pyridine | | |
| | | carboxylate | | |
| Example 50 | 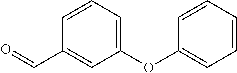 [SMILES: O=Cc1cccc(Oc2ccccc2)c1] | 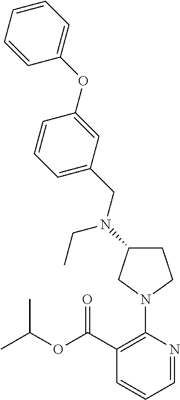 [SMILES: CCN(Cc1cccc(Oc2ccccc2)c1)[C@@H]1CCN(c2ncccc2C(=O)OC(C)C)C1] | 460.3 | 1.0 |
| | | 1-methylethyl 2-[(3R)-3- | | |
| | | (ethyl{[3-(phenyloxy) | | |
| | | phenyl]methyl}amino)-1- | | |
| | | pyrrolidinyl]-3-pyridine | | |
| | | carboxylate | | |

### Table 2

Following the procedure as described above in the preparation of 1-methylethyl 2-{methyl[(3R)-1-({2-[(trifluoromethyl)oxy]phenyl}methyl)-3-pyrrolidinyl]amino}-3-pyridinecarboxylate, 1-methylethyl 2-{ethyl[(2R)-2-(ethylamino)propyl]amino}-3-pyridine carboxylate (25 mg, 0.09 mmol) was reacted with the appropriate aldehyde or ketone to yield the examples listed in Table II.

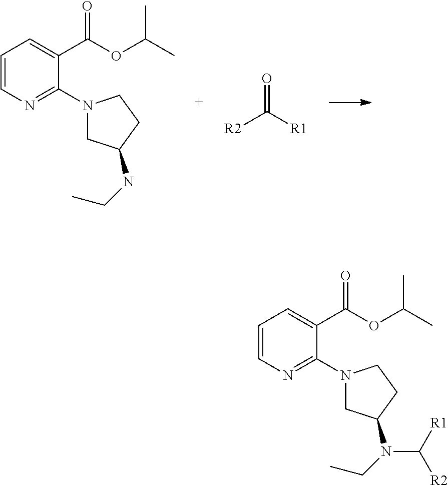

TABLE II

| | | | LC-MS | |
|---|---|---|---|---|
| | | | m/z | |
| Ex- | Aldehyde | | (M + | RT |
| ample | or Ketone | Product | H)⁺ | (min) |
| Ex- ample 51 | 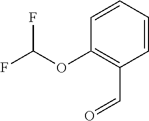 [SMILES: O=Cc1ccccc1OC(F)F] | 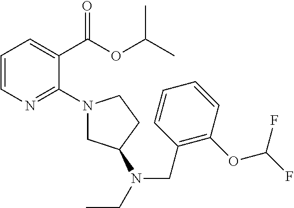 [SMILES: CCN(Cc1ccccc1OC(F)F)[C@@H]1CCN(c2ncccc2C(=O)OC(C)C)C1] | 434.21 | 0.9 |
| | | 1-methylethyl 2-{(3R)-3- | | |
| | | [({2-[(difluoromethyl) | | |
| | | oxy]phenyl}methyl) | | |
| | | (ethyl)amino]-1- | | |
| | | pyrrolidinyl}-3- | | |
| | | pyridinecarboxylate | | |
| Ex- ample 52 | 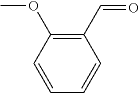 | 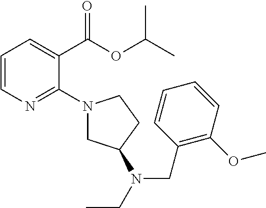 [SMILES: CCN(Cc1ccccc1OC)[C@@H]1CCN(c2ncccc2C(=O)OC(C)C)C1] | 398.15 | 0.8 |
| | | 1-methylethyl 2-[(3R)-3- | | |
| | | (ethyl{[2-(methyloxy) | | |
| | | phenyl]methyl}amino)- | | |
| | | 1-pyrrolidinyl]-3- | | |
| | | pyridinecarboxylate | | |
| Ex- ample 53 | 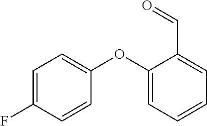 [SMILES: O=Cc1ccccc1Oc1ccc(F)cc1] | 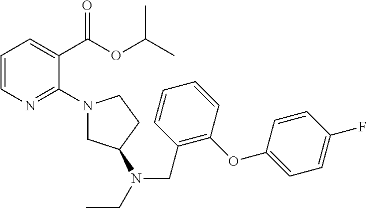 [SMILES: CCN(Cc1ccccc1Oc1ccc(F)cc1)[C@@H]1CCN(c2ncccc2C(=O)OC(C)C)C1] | 478.18 | 1.0 |
| | | 1-methylethyl 2-{(3R)-3- | | |
| | | [ethyl({2-[(4-fluoro- | | |
| | | phenyl)oxy]phenyl}meth- | | |
| | | yl)amino]-1-pyrrolidinyl}- | | |
| | | 3-pyridinecarboxylate | | |
| Ex- ample 54 | 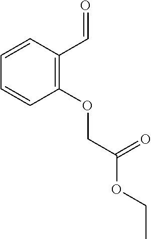 | 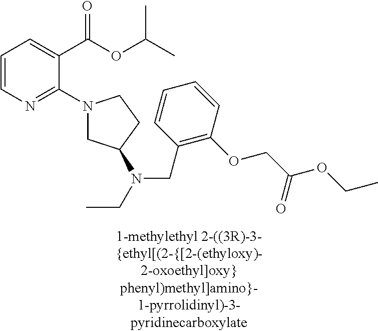 [SMILES: CCOC(=O)COc1ccccc1CN(CC)[C@@H]1CCN(c2ncccc2C(=O)OC(C)C)C1] | 470.36 | 1.0 |
| Ex- ample 55 | 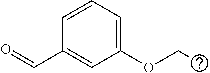 | 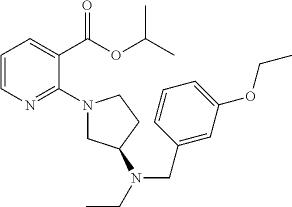 [SMILES: CCOc1cccc(CN(CC)[C@@H]2CCN(c3ncccc3C(=O)OC(C)C)C2)c1] | 412.13 | 0.9 |
| | | 1-methylethyl 2-[(3R)-3- | | |
| | | (ethyl{[3-(ethyloxy) | | |
| | | phenyl]methyl}amino)- | | |
| | | 1-pyrrolidinyl]-3- | | |
| | | pyridinecarboxylate | | |
| Ex- ample 56 | 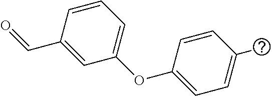 [SMILES: Cc1ccc(Oc2cccc(C=O)c2)cc1] | 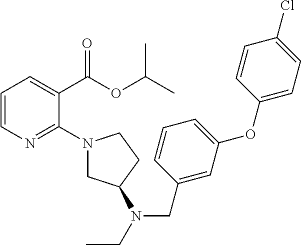 [SMILES: CCN(Cc1cccc(Oc2ccc(Cl)cc2)c1)[C@@H]1CCN(c2ncccc2C(=O)OC(C)C)C1] | 494.23 | 1.1 |
| | | 1-methylethyl 2-{(3R)-3- | | |
| | | [({3-[(4-chlorophenyl) | | |
| | | oxy]phenyl}methyl)(ethyl) | | |
| | | amino]-1-pyrrolidinyl}- | | |
| | | 3-pyridinecarboxylate | | |
| Ex- ample 57 | 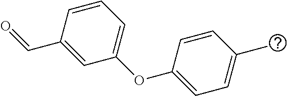 [SMILES: Cc1ccc(Oc2cccc(C=O)c2)cc1] | 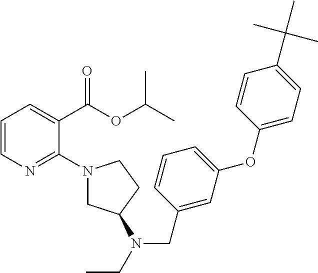 [SMILES: CCN(Cc1cccc(Oc2ccc(C(C)(C)C)cc2)c1)[C@@H]1CCN(c2ncccc2C(=O)OC(C)C)C1] | 516.39 | 1.2 |
| | | 1-methylethyl 2-{(3R)-3- | | |
| | | [[(3-{[4-(1,1-dimethyl- | | |
| | | ethyl)phenyl]oxy}phenyl) | | |
| | | methyl](ethyl)amino]-1- | | |
| | | pyrrolidinyl}-3- | | |
| | | pyridinecarboxylate | | |
| Ex- ample 58 | 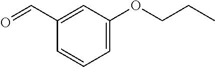 | 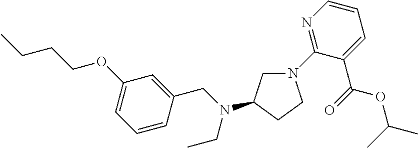 [SMILES: CCCCOc1cccc(CN(CC)[C@@H]2CCN(c3ncccc3C(=O)OC(C)C)C2)c1] | 440.27 | 1.0 |
| | | 1-methylethyl 2-{(3R)-3- | | |
| | | [{[3-(butyloxy)phenyl] | | |
| | | methyl}(ethyl)amino]-1- | | |
| | | pyrrolidinyl}-3- | | |
| | | pyridinecarboxylate | | |
| Ex- ample 59 | 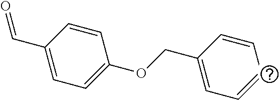 [SMILES: O=Cc1ccc(OCc2ccccc2)cc1] | 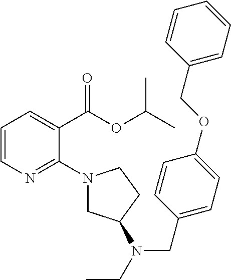 [SMILES: CCN(Cc1ccc(OCc2ccccc2)cc1)[C@@H]1CCN(c2ncccc2C(=O)OC(C)C)C1] | 474.3 | 1.0 |
| | | 1-methylethyl 2-{(3R)-3- | | |
| | | [ethyl({4-[(phenyl- | | |
| | | methyl)oxy]phenyl}meth- | | |
| | | yl)amino]-1-pyrrolidinyl}- | | |
| | | 3-pyridinecarboxylate | | |
| Ex- ample 60 | 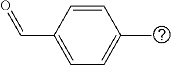 | 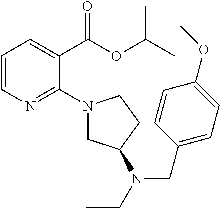 [SMILES: CCN(Cc1ccc(OC)cc1)[C@@H]1CCN(c2ncccc2C(=O)OC(C)C)C1] | 398.15 | 0.8 |
| | | 1-methylethyl 2-[(3R)-3- | | |
| | | (ethyl{[4-(methyloxy) | | |
| | | phenyl]methyl}amino)- | | |
| | | 1-pyrrolidinyl]-3- | | |
| | | pyridinecarboxylate | | |
| Ex- ample 61 | 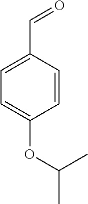 | 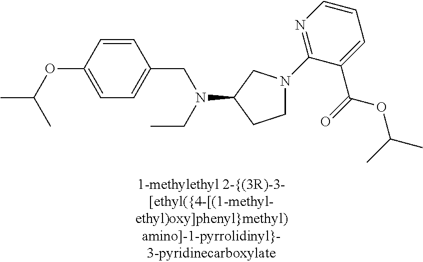 [SMILES: CCN(Cc1ccc(OC(C)C)cc1)[C@@H]1CCN(c2ncccc2C(=O)OC(C)C)C1] | 426.27 | 1.2 |
| Ex- ample 62 | 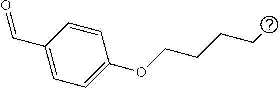 | 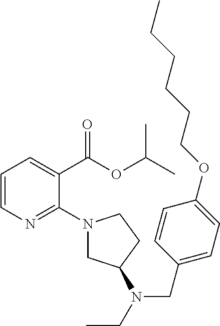 [SMILES: CCCCCCOc1ccc(CN(CC)[C@@H]2CCN(c3ncccc3C(=O)OC(C)C)C2)cc1] | 468.46 | 1.2 |
| | | 1-methylethyl 2-[(3R)-3- | | |
| | | (ethyl{[4-(hexyloxy) | | |
| | | phenyl]methyl}amino)- | | |
| | | 1-pyrrolidinyl]-3- | | |
| | | pyridinecarboxylate | | |
| Ex- ample 63 | 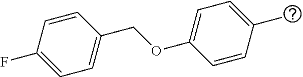 [SMILES: Cc1ccc(OCc2ccc(F)cc2)cc1] | 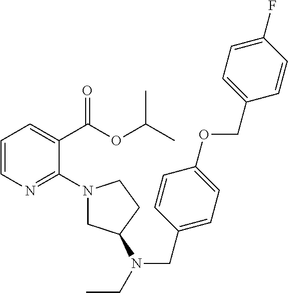 [SMILES: CCN(Cc1ccc(OCc2ccc(F)cc2)cc1)[C@@H]1CCN(c2ncccc2C(=O)OC(C)C)C1] | 492.32 | 1.0 |
| | | 1-methylethyl 2-((3R)-3- | | |
| | | {ethyl[(4-{[(4-fluoro- | | |
| | | phenyl)methyl]oxy}phen- | | |
| | | yl)methyl]amino}-1- | | |
| | | pyrrolidinyl)-3- | | |
| | | pyridinecarboxylate | | |
| Ex- ample 64 | 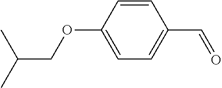 | 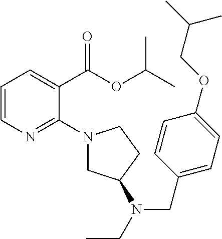 [SMILES: CCN(Cc1ccc(OCC(C)C)cc1)[C@@H]1CCN(c2ncccc2C(=O)OC(C)C)C1] | 440.29 | 1.0 |
| | | 1-methylethyl 2-{(3R)-3- | | |
| | | [ethyl({4-[(2- | | |
| | | methylpropyl)oxy]phenyl} | | |
| | | methyl)amino]-1- | | |
| | | pyrrolidinyl}-3- | | |
| | | pyridinecarboxylate | | |
| Ex- ample 65 | 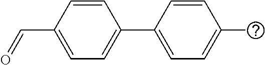 | 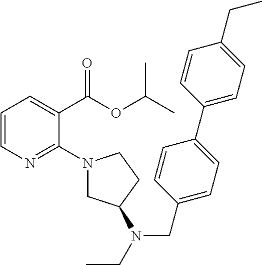 [SMILES: CCc1ccc(-c2ccc(CN(CC)[C@@H]3CCN(c4ncccc4C(=O)OC(C)C)C3)cc2)cc1] | 472.32 | 1.1 |
| | | 1-methylethyl 2-((3R)-3- | | |
| | | {ethyl[(4′-ethyl-4- | | |
| | | biphenylyl)methyl]amino}- | | |
| | | 1-pyrrolidinyl)-3- | | |
| | | pyridinecarboxylate | | |
| Ex- ample 66 | 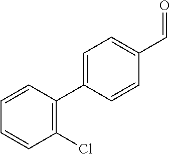 [SMILES: O=Cc1ccc(-c2ccccc2Cl)cc1] | 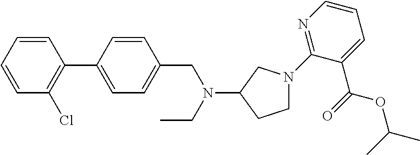 [SMILES: CCN(Cc1ccc(-c2ccccc2Cl)cc1)C1CCN(c2ncccc2C(=O)OC(C)C)C1] | 478.25 | 1.0 |
| | | 1-methylethyl 2-{(3R)-3- | | |
| | | [[(2′-chloro-4- | | |
| | | biphenylyl)methyl](ethyl) | | |
| | | amino]-1-pyrrolidinyl}- | | |
| | | 3-pyridinecarboxylate | | |
| Ex- ample 67 | 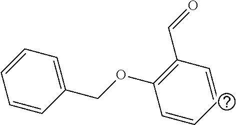 [SMILES: O=Cc1ccccc1OCc1ccccc1] | 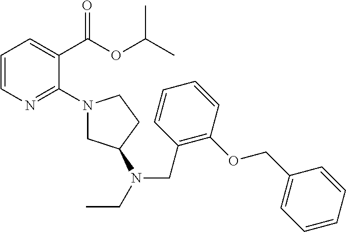 [SMILES: CCN(Cc1ccccc1OCc1ccccc1)[C@@H]1CCN(c2ncccc2C(=O)OC(C)C)C1] | 474.27 | 1.0 |
| | | 1-methylethyl 2-{(3R)-3- | | |
| | | [ethyl({2- | | |
| | | [(phenylmethyl)oxy]phen- | | |
| | | yl}methyl)amino]-1- | | |
| | | pyrrolidinyl}-3- | | |
| | | pyridinecarboxylate | | |
| Ex- ample 68 | 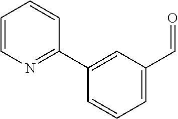 [SMILES: O=Cc1cccc(-c2ccccn2)c1] | 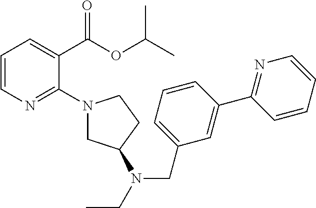 [SMILES: CCN(Cc1cccc(-c2ccccn2)c1)[C@@H]1CCN(c2ncccc2C(=O)OC(C)C)C1] | 445.29 | 0.8 |
| | | 1-methylethyl 2-[(3R)-3- | | |
| | | (ethyl{[3-(2-pyridinyl) | | |
| | | phenyl]methyl}amino)- | | |
| | | 1-pyrrolidinyl]-3- | | |
| | | pyridinecarboxylate | | |
| Ex- ample 69 | 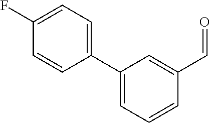 [SMILES: O=Cc1cccc(-c2ccc(F)cc2)c1] | 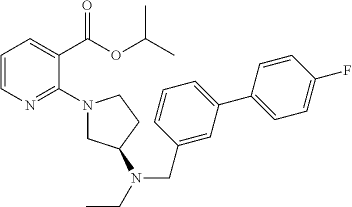 [SMILES: CCN(Cc1cccc(-c2ccc(F)cc2)c1)[C@@H]1CCN(c2ncccc2C(=O)OC(C)C)C1] | 462.28 | 1.0 |
| | | 1-methylethyl 2-((3R)-3- | | |
| | | {ethyl[(4′-fluoro-3- | | |
| | | biphenylyl)methyl]amino}- | | |
| | | 1-pyrrolidinyl)-3- | | |
| | | pyridinecarboxylate | | |
| Ex- ample 70 | 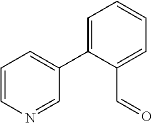 [SMILES: O=Cc1ccccc1-c1cccnc1] | 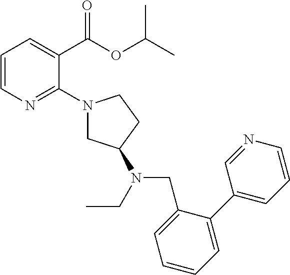 [SMILES: CCN(Cc1ccccc1-c1cccnc1)[C@@H]1CCN(c2ncccc2C(=O)OC(C)C)C1] | 445.29 | 0.7 |
| | | 1-methylethyl 2-[(3R)-3- | | |
| | | (ethyl{[2-(3-pyridinyl) | | |
| | | phenyl]methyl}amino)- | | |
| | | 1-pyrrolidinyl]-3- | | |
| | | pyridinecarboxylate | | |
| Ex- ample 71 | 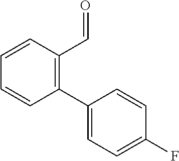 [SMILES: O=Cc1ccccc1-c1ccc(F)cc1] | 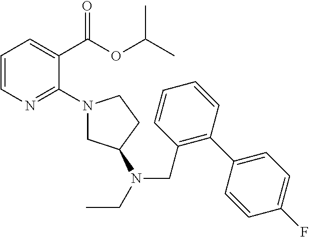 [SMILES: CCN(Cc1ccccc1-c1ccc(F)cc1)[C@@H]1CCN(c2ncccc2C(=O)OC(C)C)C1] | 462.29 | 1.0 |
| | | 1-methylethyl 2-((3R)-3- | | |
| | | {ethyl[(4′-fluoro-2- | | |
| | | biphenylyl)methyl]amino}- | | |
| | | 1-pyrrolidinyl)-3- | | |
| | | pyridinecarboxylate | | |
| Ex- ample 72 | 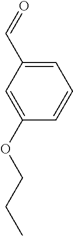 | 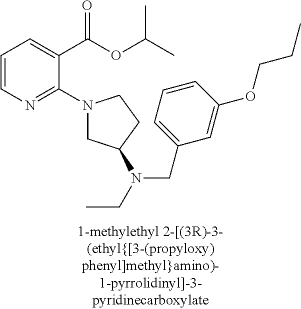 [SMILES: CCCOc1cccc(CN(CC)[C@@H]2CCN(c3ncccc3C(=O)OC(C)C)C2)c1] | 426.27 | 1.0 |
| Ex- ample 73 | 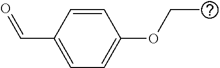 | 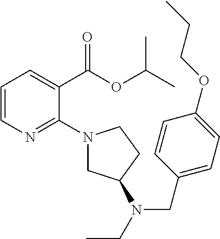 [SMILES: CCCOc1ccc(CN(CC)[C@@H]2CCN(c3ncccc3C(=O)OC(C)C)C2)cc1] | 426.28 | 1.0 |
| | | 1-methylethyl 2-[(3R)-3- | | |
| | | (ethyl{[4-(propyloxy) | | |
| | | phenyl]methyl}amino)- | | |
| | | 1-pyrrolidinyl]-3- | | |
| | | pyridinecarboxylate | | |

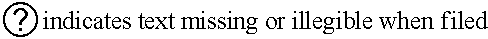

### Table 3

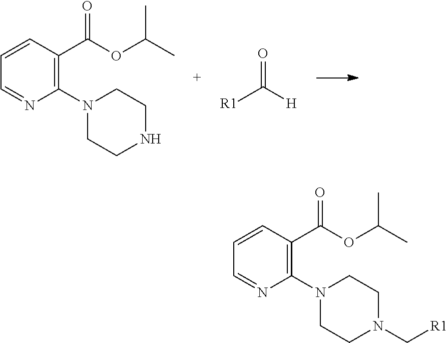

A solution of 1-methylethyl 2-(1-piperazinyl)-3-pyridinecarboxylate (625 mg, 2.5 mmol) in dimethyl sulfoxide (DMSO) (37.5 ml) was dispensed into 25 A-vials containing benzaldehydes (0.2 mmol, purchased from Sigma Aldrich) followed by addition of acetic acid (5 L, 0.087 mmol). The reaction was stirred for 4 h in a VX-2500 Multi-Tube Vortexer. MP-B(OAc)₃ (83 mg, 0.201 mmol) was then added and the reaction was stirred overnight in a VX-2500 Multi-Tube Vortexer. Starting material remained so sodium triacetoxyborohydride (50.0 mg, 0.236 mmol) was added to all the reaction mixtures. These were stirred over a weekend in a VX-2500 Multi-Tube Vortexer.

The reaction mixtures were filtered using a Bohdan miniblock, concentrated then purified via preparative HPLC (Column: X-Bridge 19×100 mm 5, Mobile phase: Acetonitrile: Water 0.1% NH₄OH, Flow rate: 15 ml/min). These are shown in Table III.

TABLE III

| | | | LC-MS | |
|---|---|---|---|---|
| | Aldehyde | | m/z | RT |
| Example | (ketone) | Product Name | (M + H)⁺ | (min) |
| Example 74 | 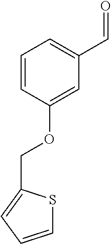 [SMILES: O=Cc1cccc(OCc2cccs2)c1] | 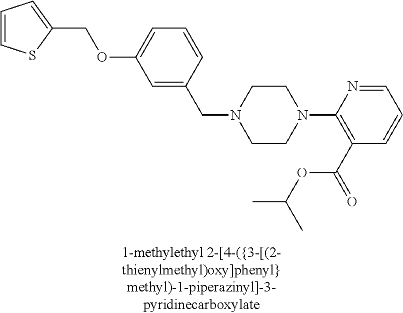 [SMILES: CC(C)OC(=O)c1cccnc1N1CCN(Cc2cccc(OCc3cccs3)c2)CC1] | 452.18 | 1.0 |
| Example 75 | 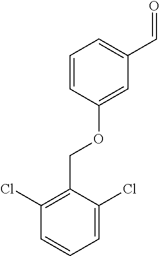 [SMILES: O=Cc1cccc(OCc2c(Cl)cccc2Cl)c1] | 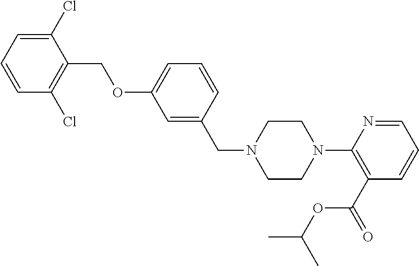 [SMILES: CC(C)OC(=O)c1cccnc1N1CCN(Cc2cccc(OCc3c(Cl)cccc3Cl)c2)CC1] | 514.32 | 1.1 |
| | | 1-methylethyl 2-{4-[(3- | | |
| | | {[(2,6- | | |
| | | dichlorophenyl)methyl]oxy} | | |
| | | phenyl)methyl]-1- | | |
| | | piperazinyl}-3- | | |
| | | pyridinecarboxylate | | |
| Example 76 | 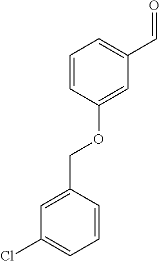 [SMILES: O=Cc1cccc(OCc2cccc(Cl)c2)c1] | 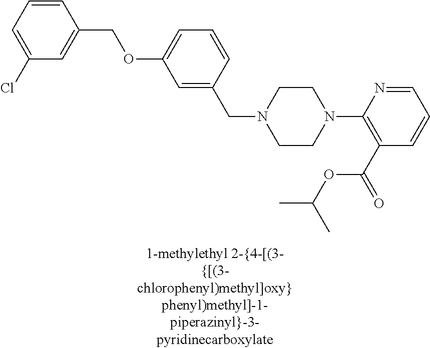 [SMILES: CC(C)OC(=O)c1cccnc1N1CCN(Cc2cccc(OCc3cccc(Cl)c3)c2)CC1] | 480.13 | 1.1 |
| Example 77 | 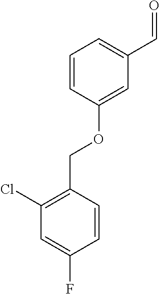 [SMILES: O=Cc1cccc(OCc2ccc(F)cc2Cl)c1] | 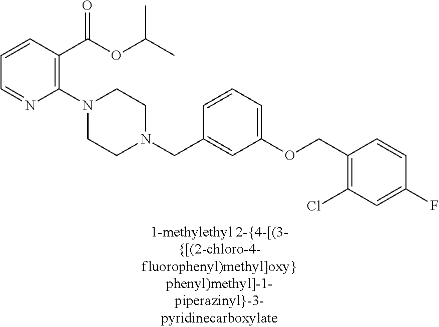 [SMILES: CC(C)OC(=O)c1cccnc1N1CCN(Cc2cccc(OCc3ccc(F)cc3Cl)c2)CC1] | 498.42 | 1.1 |
| Example 78 | 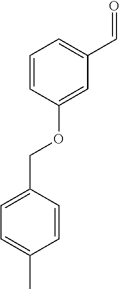 [SMILES: Cc1ccc(COc2cccc(C=O)c2)cc1] | 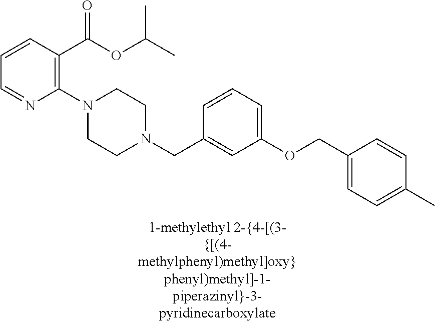 [SMILES: Cc1ccc(COc2cccc(CN3CCN(c4ncccc4C(=O)OC(C)C)CC3)c2)cc1] | 460.28 | 1.053 |
| Example 79 | 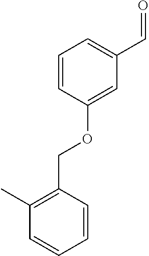 [SMILES: Cc1ccccc1COc1cccc(C=O)c1] | 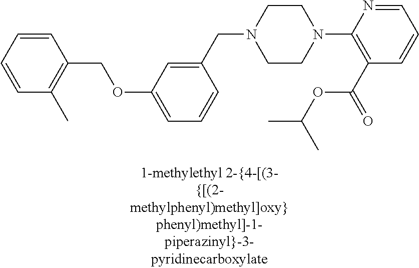 [SMILES: Cc1ccccc1COc1cccc(CN2CCN(c3ncccc3C(=O)OC(C)C)CC2)c1] | 460.29 | 1.0 |
| Example 80 | 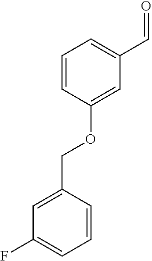 [SMILES: O=Cc1cccc(OCc2cccc(F)c2)c1] | 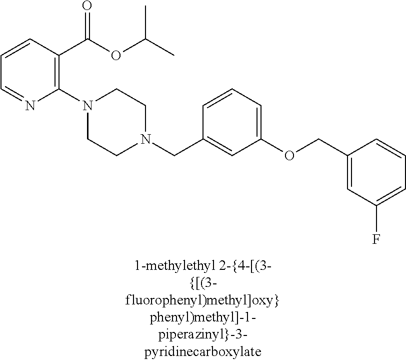 [SMILES: CC(C)OC(=O)c1cccnc1N1CCN(Cc2cccc(OCc3cccc(F)c3)c2)CC1] | 464.31 | 1.0 |
| Example 81 | 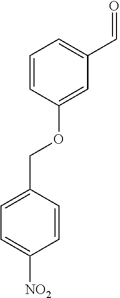 [SMILES: O=Cc1cccc(OCc2ccc([N+](=O)[O-])cc2)c1] | 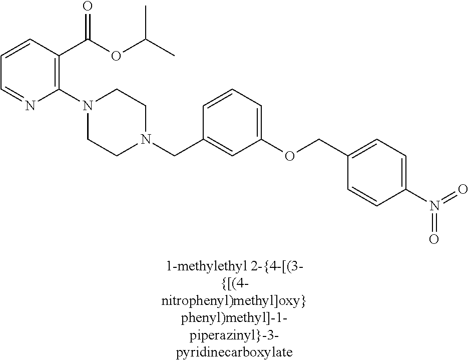 [SMILES: CC(C)OC(=O)c1cccnc1N1CCN(Cc2cccc(OCc3ccc([N+](=O)[O-])cc3)c2)CC1] | 491.31 | 1.0 |
| Example 82 | 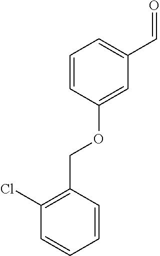 [SMILES: O=Cc1cccc(OCc2ccccc2Cl)c1] | 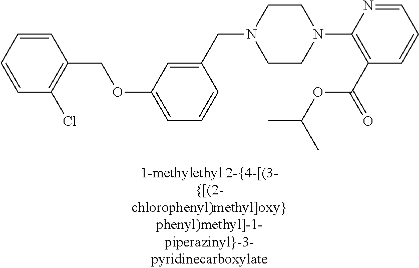 [SMILES: CC(C)OC(=O)c1cccnc1N1CCN(Cc2cccc(OCc3ccccc3Cl)c2)CC1] | 480.15 | 1.0 |
| Example 83 | 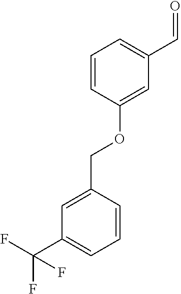 [SMILES: O=Cc1cccc(OCc2cccc(C(F)(F)F)c2)c1] | 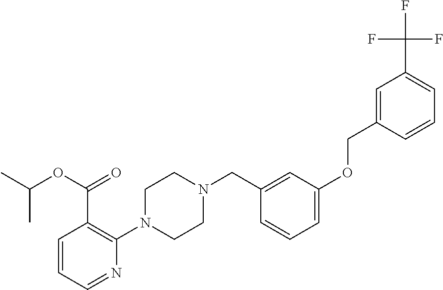 [SMILES: CC(C)OC(=O)c1cccnc1N1CCN(Cc2cccc(OCc3cccc(C(F)(F)F)c3)c2)CC1] | 514.44 | 1.1 |
| | | 1-methylethyl 2-(4-{[3- | | |
| | | ({[3- | | |
| | | (trifluoromethyl)phenyl] | | |
| | | methyl}oxy)phenyl]methyl}-1- | | |
| | | piperazinyl)-3- | | |
| | | pyridinecarboxylate | | |
| Example 84 | 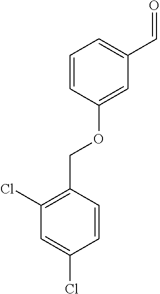 [SMILES: O=Cc1cccc(OCc2ccc(Cl)cc2Cl)c1] | 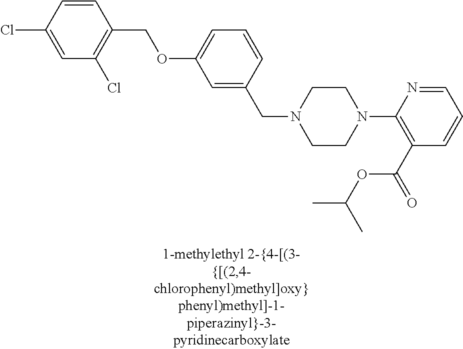 [SMILES: CC(C)OC(=O)c1cccnc1N1CCN(Cc2cccc(OCc3ccc(Cl)cc3Cl)c2)CC1] | 515.1 | 0.9 |
| Example 85 | 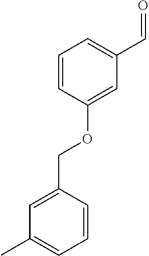 [SMILES: Cc1cccc(COc2cccc(C=O)c2)c1] | 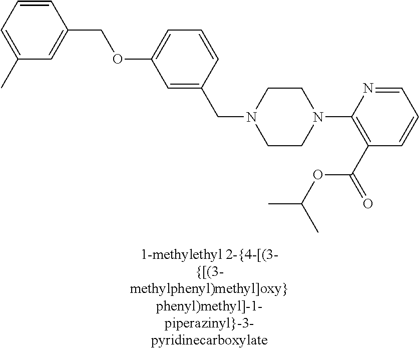 [SMILES: Cc1cccc(COc2cccc(CN3CCN(c4ncccc4C(=O)OC(C)C)CC3)c2)c1] | 461.1 | 0.9 |
| Example 86 | 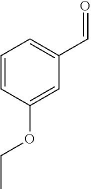 | 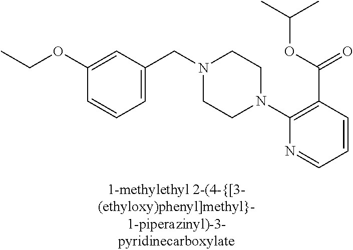 [SMILES: CCOc1cccc(CN2CCN(c3ncccc3C(=O)OC(C)C)CC2)c1] | 385 | 0.8 |
| Example 87 | 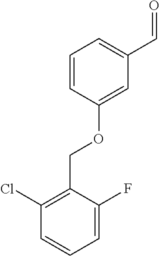 [SMILES: O=Cc1cccc(OCc2c(F)cccc2Cl)c1] | 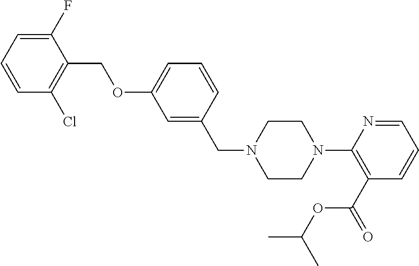 [SMILES: CC(C)OC(=O)c1cccnc1N1CCN(Cc2cccc(OCc3c(F)cccc3Cl)c2)CC1] | 499.1 | 0.8 |
| | | 1-methylethyl 2-{4-[(3- | | |
| | | {[(2-chloro-6- | | |
| | | fluorophenyl)methyl]oxy} | | |
| | | phenyl)methyl]-1- | | |
| | | piperazinyl}-3- | | |
| | | pyridinecarboxylate | | |
| Example 88 | 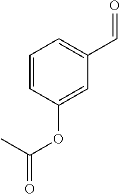 | 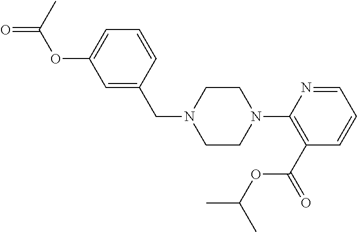 [SMILES: CC(=O)Oc1cccc(CN2CCN(c3ncccc3C(=O)OC(C)C)CC2)c1] | 399 | 0.7 |
| | | 1-methylethyl 2-(4-{[3- | | |
| | | (acetyloxy)phenyl]methyl}- | | |
| | | 1-piperazinyl)-3- | | |
| | | pyridinecarboxylate | | |
| Example 89 | 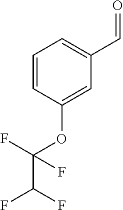 [SMILES: O=Cc1cccc(OC(F)(F)C(F)F)c1] | 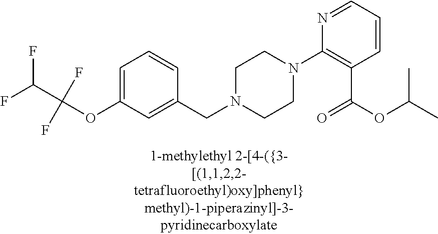 [SMILES: CC(C)OC(=O)c1cccnc1N1CCN(Cc2cccc(OC(F)(F)C(F)F)c2)CC1] | 457 | 0.8 |
| Example 90 | 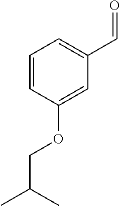 | 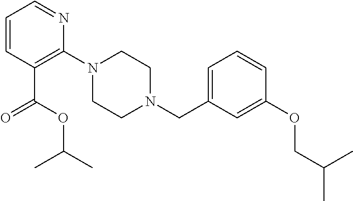 [SMILES: CC(C)COc1cccc(CN2CCN(c3ncccc3C(=O)OC(C)C)CC2)c1] | 413 | 0.8 |
| | | 1-methylethyl 2-[4-({3-[(2- | | |
| | | methylpropyl)oxy]phenyl} | | |
| | | methyl)-1-piperazinyl]-3- | | |
| | | pyridinecarboxylate | | |
| Example 91 | 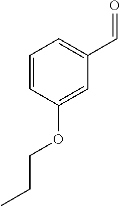 | 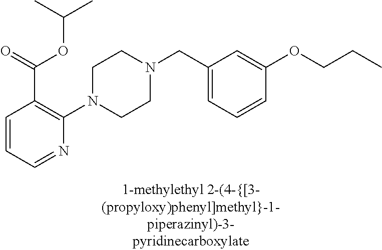 [SMILES: CCCOc1cccc(CN2CCN(c3ncccc3C(=O)OC(C)C)CC2)c1] | 399 | 0.8 |
| Example 92 | 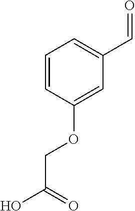 [SMILES: O=Cc1cccc(OCC(=O)O)c1] | 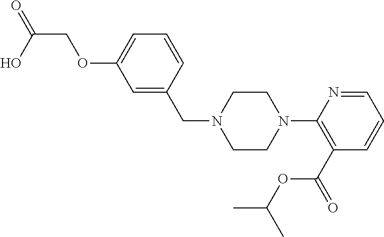 [SMILES: CC(C)OC(=O)c1cccnc1N1CCN(Cc2cccc(OCC(=O)O)c2)CC1] | 415 | 0.6 |
| | | [(3-{[4-(3-{[(1- | | |
| | | methylethyl)oxy]carbonyl}- | | |
| | | 2-pyridinyl)-1- | | |
| | | piperazinyl]methyl}phenyl) | | |
| | | oxy]acetic acid | | |
| Example 93 | 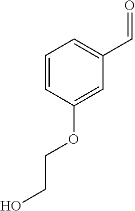 [SMILES: O=Cc1cccc(OCCO)c1] | 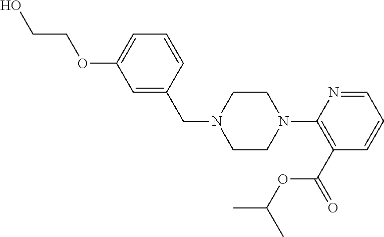 [SMILES: CC(C)OC(=O)c1cccnc1N1CCN(Cc2cccc(OCCO)c2)CC1] | 401 | 0.6 |
| | | 1-methylethyl 2-[4-({3-[(2- | | |
| | | hydroxyethyl)oxy]phenyl} | | |
| | | methyl)-1-piperazinyl]-3- | | |
| | | pyridinecarboxylate | | |
| Example 94 | 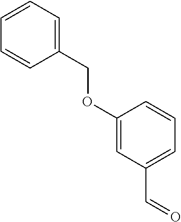 [SMILES: O=Cc1cccc(OCc2ccccc2)c1] | 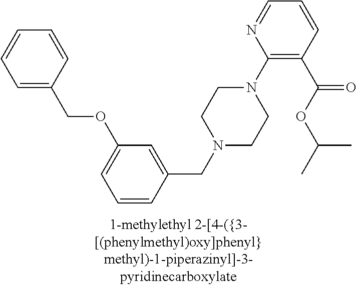 [SMILES: CC(C)OC(=O)c1cccnc1N1CCN(Cc2cccc(OCc3ccccc3)c2)CC1] | 447.1 | 0.8 |
| Example 95 | 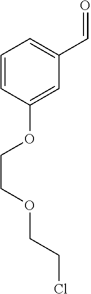 [SMILES: O=Cc1cccc(OCCOCCCl)c1] | 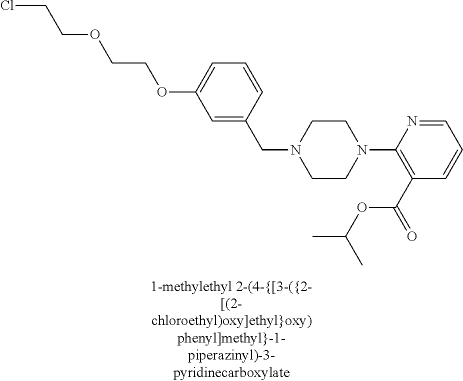 [SMILES: CC(C)OC(=O)c1cccnc1N1CCN(Cc2cccc(OCCOCCCl)c2)CC1] | 463.1 | 0.8 |
| Example 96 | 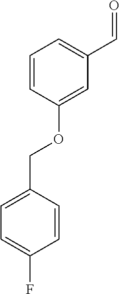 [SMILES: O=Cc1cccc(OCc2ccc(F)cc2)c1] | 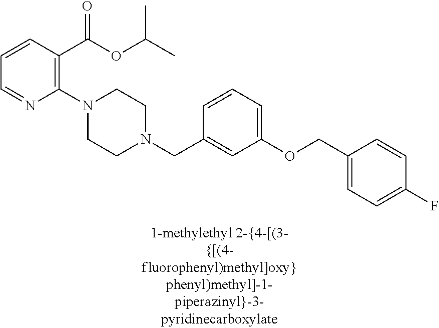 [SMILES: CC(C)OC(=O)c1cccnc1N1CCN(Cc2cccc(OCc3ccc(F)cc3)c2)CC1] | 465.1 | 0.8 |
| Example 97 | 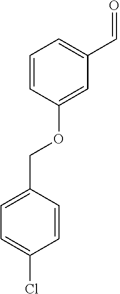 [SMILES: O=Cc1cccc(OCc2ccc(Cl)cc2)c1] | 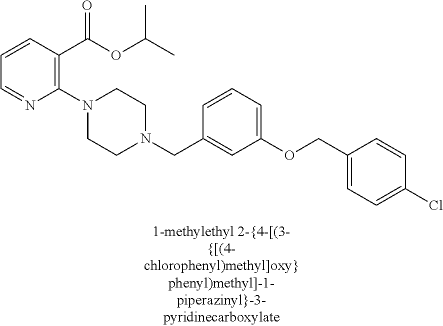 [SMILES: CC(C)OC(=O)c1cccnc1N1CCN(Cc2cccc(OCc3ccc(Cl)cc3)c2)CC1] | 481.1 | 0.9 |

Table 4

### Example 98

### 1-methylethyl 2-[4-(phenylmethyl)-1-piperazinyl]-4-(phenyloxy)-3-pyridinecarboxylate

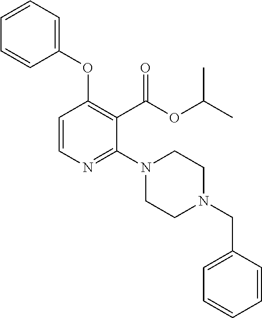 [SMILES: CC(C)OC(=O)c1c(Oc2ccccc2)ccnc1N1CCN(Cc2ccccc2)CC1]

Added the 1-methylethyl 4-iodo-2-[4-(phenylmethyl)-1-piperazinyl]-3-pyridinecarboxylate (30.0 mg, 0.064 mmol), phenol (12.1 mg, 0.129 mmol), Xantphos (6.2 mg, 0.013 mmol), t-BuONa (9.3 mg, 0.097 mmol) and Palladium(II) acetate (1.45 mg, 10 mol %) in Toluene (3.0 mL) in a 5 mL reaction vial, and then stirred the mixture for 30 min at room temperature under nitrogen atmosphere. The stirring was kept for overnight at 100° C. Pretreated a StratoSpheres PL-Thiol MP SPE column with Methanol, filtered reaction mixture through column, washed with methanol. Concentrated and obtained the crude product, which was dissolved in DMSO, and purified on a Gilson HPLC (Sunfire 30×150 mm, 5 um preparatory column), eluting at 40 mL/min with linear gradient running from 30% to 100% acetonitrile and 0.1% aqueous NH₄OH over 25 min. The desired fractions were concentrated under a stream of nitrogen at 50° C., giving the desired product (8.28 mg, 26.5%).

LC/MS: m/z=432.1[M+H]⁺, Ret. Time: 1.00 min.

### Example 99

### 1-methylethyl4-[(2-fluorophenyl)amino]-2-[4-(phenylmethyl)-1-piperazinyl]-3-pyridinecarboxylate

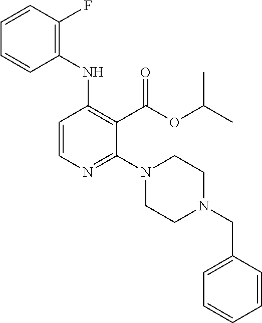 [SMILES: CC(C)OC(=O)c1c(Nc2ccccc2F)ccnc1N1CCN(Cc2ccccc2)CC1]

Added the 1-methylethyl 4-iodo-2-[4-(phenylmethyl)-1-piperazinyl]-3-pyridine carboxylate (30.0 mg, 0.064 mmol), Anilines (0.148 mmol), Xantphos (6.2 mg, 0.013 mmol), Potassium phosphate (41.1 mg, 0.193 mmol) and Palladium(II) acetate (1.45 mg, 10 mol %) in 3.0 mL Toluene in a 5 mL reaction vial, and then stirred the mixture for 30 min at room temperature under nitrogen atmosphere. The stirring was kept for overnight at 102° C. Pretreated a StratoSpheres PL-Thiol MP SPE column with methanol, filtered reaction mixture through column, washed with methanol. Concentrated to give the crude product, which was dissolved in DMSO, and purified on a Gilson HPLC (Sunfire 30×150 mm, 5 um preparatory column), eluting at 40 mL/min with linear gradient running from 30% to 100% acetonitrile and 0.1% aqueous

NH₄OH over 25 min. The desired fractions were concentrated under a stream of nitrogen at 50° C., giving the desired product (6.47 mg, 22.37%). LC/MS: m/z=449.1 [M+H]⁺, Ret. Time: 0.84 min.

Following the procedure as described above in the preparation of 1-methylethyl 4-[(2-fluorophenyl)amino]-2-[4-(phenylmethyl)-1-piperazinyl]-3-pyridinecarboxylate, 1-methylethyl 4-iodo-2-[4-(phenylmethyl)-1-piperazinyl]-3-pyridinecarboxylate (30.0 mg, 0.064 mmol) was reacted with the appropriate aryl anilines (0.148 mmol) to yield the examples listed in Table IV.

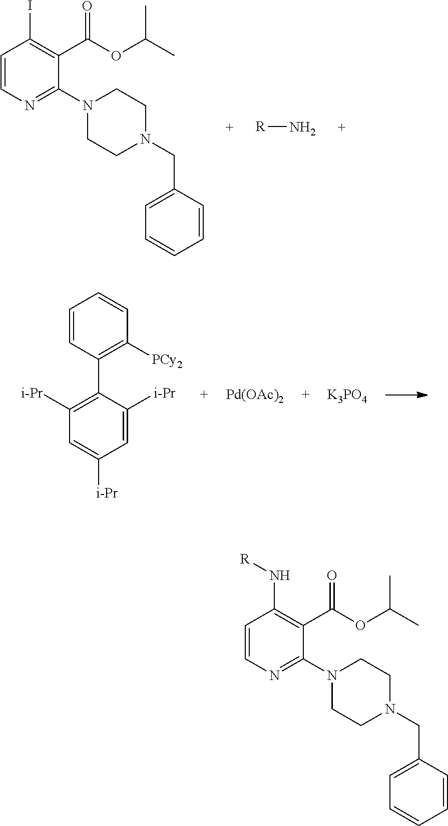

TABLE IV

| | | | LC-MS | |
|---|---|---|---|---|
| | | | m/z | RT |
| Example | Aniline | Product | (M + H)⁺ | (min) |
| Example 100 | 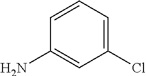 | 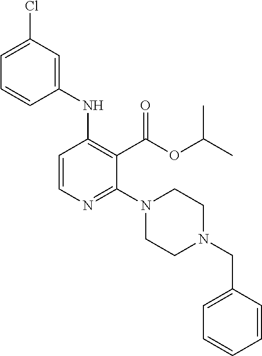 [SMILES: CC(C)OC(=O)c1c(Nc2cccc(Cl)c2)ccnc1N1CCN(Cc2ccccc2)CC1] 1-methylethyl 4-[(3- chlorophenyl)amino]-2- [4-(phenylmethyl)-1- piperazinyl]-3- pyridinecarboxylate | 465.1 | 0.9 |
| Example 101 | 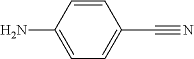 | 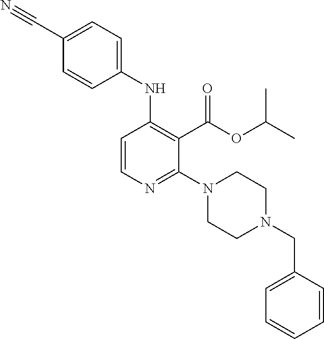 [SMILES: CC(C)OC(=O)c1c(Nc2ccc(C#N)cc2)ccnc1N1CCN(Cc2ccccc2)CC1] 1-methylethyl 4-[(4- cyanophenyl)amino]-2- [4-(phenylmethyl)-1- piperazinyl]-3- pyridinecarboxylate | 456.1 | 0.8 |
| Example 102 | 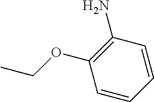 | 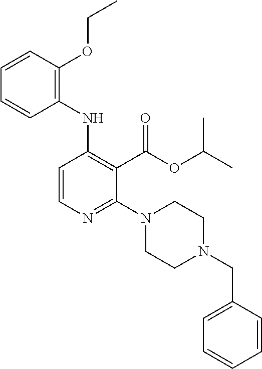 [SMILES: CCOc1ccccc1Nc1ccnc(N2CCN(Cc3ccccc3)CC2)c1C(=O)OC(C)C] 1-methylethyl 4-{[2- (ethyloxy)phenyl]amino}- 2-[4-(phenylmethyl)- 1-piperazinyl]-3- pyridinecarboxylate | 475.1 | 0.8 |
| Example 103 | 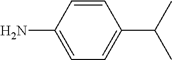 | 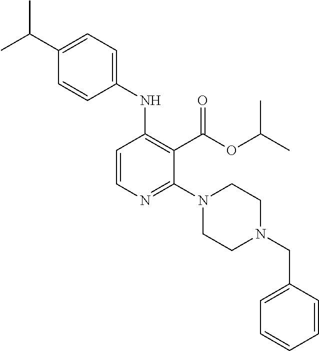 [SMILES: CC(C)OC(=O)c1c(Nc2ccc(C(C)C)cc2)ccnc1N1CCN(Cc2ccccc2)CC1] 1-methylethyl 4-{[4-(1- methylethyl)phenyl] amino}-2-[4- (phenylmethyl)-1- piperazinyl]-3- pyridinecarboxylate | 473.2 | 0.9 |
| Example 104 | 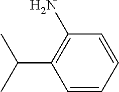 | 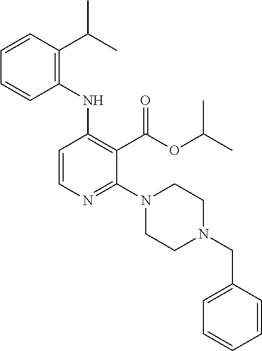 [SMILES: CC(C)OC(=O)c1c(Nc2ccccc2C(C)C)ccnc1N1CCN(Cc2ccccc2)CC1] 1-methylethyl 4-{[2-(1- methylethyl)phenyl] amino}-2-[4- (phenylmethyl)-1- piperazinyl]-3- pyridinecarboxylate | 473.2 | 0.9 |
| Example 105 | 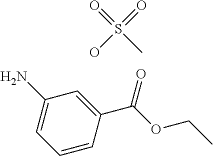 | 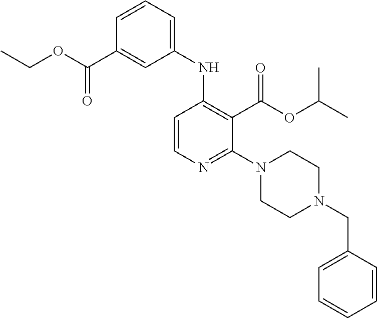 [SMILES: CCOC(=O)c1cccc(Nc2ccnc(N3CCN(Cc4ccccc4)CC3)c2C(=O)OC(C)C)c1] 1-methylethyl 4-({3- [(ethyloxy)carbonyl] phenyl}amino)-2-[4- (phenylmethyl)-1- piperazinyl]-3- pyridinecarboxylate | 503.2 | 0.9 |
| Example 106 | 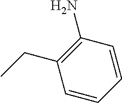 | 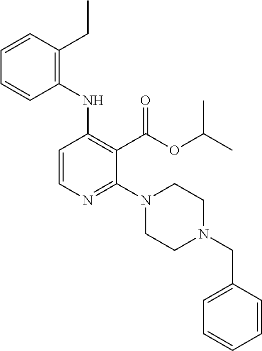 [SMILES: CCc1ccccc1Nc1ccnc(N2CCN(Cc3ccccc3)CC2)c1C(=O)OC(C)C] 1-methylethyl 4-[(2- ethylphenyl)amino]-2- [4-(phenylmethyl)-1- piperazinyl]-3- pyridinecarboxylate | 459.1 | 0.9 |
| Example 107 | 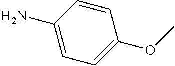 | 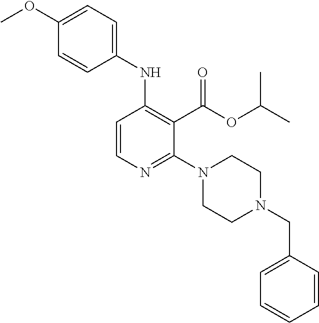 [SMILES: COc1ccc(Nc2ccnc(N3CCN(Cc4ccccc4)CC3)c2C(=O)OC(C)C)cc1] 1-methylethyl 4-{[4- (methyloxy)phenyl] amino}-2-[4- (phenylmethyl)-1- piperazinyl]-3- pyridinecarboxylate | 461.1 | 0.8 |
| Example 108 | 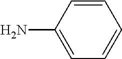 | 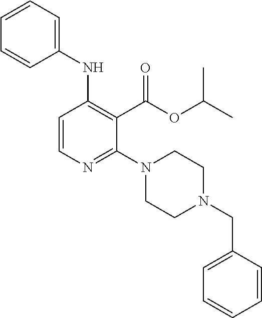 [SMILES: CC(C)OC(=O)c1c(Nc2ccccc2)ccnc1N1CCN(Cc2ccccc2)CC1] 1-methylethyl 4- (phenylamino)-2-[4- (phenylmethyl)-1- piperazinyl]-3- pyridinecarboxylate | 431.1 | 0.8 |

### Table 5

### Example 109

### 1-methylethyl 2-[4-(phenylmethyl)-1-piperazinyl]-4-(phenylthio)-3-pyridinecarboxylate

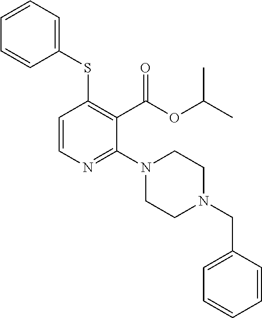 [SMILES: CC(C)OC(=O)c1c(Sc2ccccc2)ccnc1N1CCN(Cc2ccccc2)CC1]

Added 1-methylethyl 4-iodo-2-[4-(phenylmethyl)-1-piperazinyl]-3-pyridinecarboxylate (0.050 g, 0.135 mmol), copper(I) iodide (1.023 mg, 5.37 μmol) and potassium carbonate (0.030 g, 0.215 mmol) to a 5 mL microwave vial flushed with Argon. Then added Isopropanol (2.0 ml), ethylene glycol (13.34 mg, 0.215 mmol) and benzenethiol (0.236 mmol). Heated reaction to 150° C. for 20 min. Pretreated a StratoSpheres PL-Thiol MP SPE column with Methanol, filtered reaction mixture through column, washed with Methanol. Concentrated and to give the crude product, which was dissolved in DMSO, and purified on a Gilson HPLC (Sunfire 30×150 mm, 5 um preparatory column), eluting at 40 mL/min with linear gradient running from 30% to 100% acetonitrile and 0.1% aqueous NH₄OH over 25 min. The desired fractions were concentrated under a stream of nitrogen at 50° C., giving the desired product (31.69 mg, 65.9%).

LC/MS: m/z=448.1[M+H]⁺, Ret. Time: 1.03 min.

### Example 110

### 1-methylethyl 4-{[2-(methyloxy)phenyl]thio}-2-[4-(phenylmethyl)-1-piperazinyl]3-pyridine carboxylate

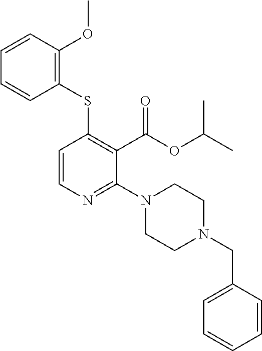 [SMILES: COc1ccccc1Sc1ccnc(N2CCN(Cc3ccccc3)CC2)c1C(=O)OC(C)C]

Added 1-methylethyl 4-iodo-2-[4-(phenylmethyl)-1-piperazinyl]-3-pyridinecarboxylate (0.050 g, 0.135 mmol), copper(I) iodide (1.023 mg, 5.37 μmol) and potassium carbonate (0.030 g, 0.215 mmol) to a 5 mL microwave vial flushed with Argon. Then added Isopropanol (2.0 ml), ethylene glycol (13.34 mg, 0.215 mmol) and 2-(methyloxy)phenyl hydrosulfide (33.1 mg, 0.236 mmol). Heated reaction to 150° C. for 20 min. Pretreated a StratoSpheres PL-Thiol MP SPE column with Methanol, filtered reaction mixture through column, washed with Methanol. Concentrated and to give the crude product, which was dissolved in DMSO, and purified on a Gilson HPLC (Sunfire 30×150 mm, 5 um preparatory column), eluting at 40 mL/min with a linear gradient running from 30% to 100% acetonitrile and 0.1% aqueous NH₄OH over 25 min. The desired fractions were concentrated under a stream of nitrogen at 50° C., giving the desired product (38 mg, 74%).

LC/MS: m/z=478.1[M+H]⁺, Ret. Time: 1.08 min.

### Example 111

### 1-methylethyl 2-[4-({2-[(2-chlorophenyl)amino]phenyl}methyl)-1-piperazinyl]-3-pyridinecarboxylate

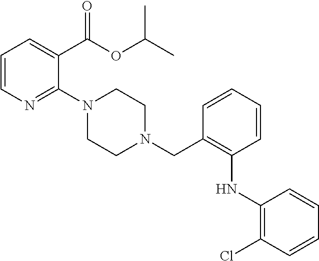 [SMILES: CC(C)OC(=O)c1cccnc1N1CCN(Cc2ccccc2Nc2ccccc2Cl)CC1]

Added the 1-methylethyl 2-{4-[(2-bromophenyl)methyl]-1-piperazinyl}-3-pyridinecarboxylate (25.0 mg, 0.060 mmol), (2-chlorophenyl)amine (0.120 mmol), XPhos (5.7 mg, 0.012 mmol), potassium phosphate (38.1 mg, 0.179 mmol) and palladium(II) acetate (1.34 mg, 10 mol %) in 3.0 mL Toluene in a 5 mL reaction vial, and then stirred the mixture for 30 min at room temperature under nitrogen atmosphere. The stirring was kept for 12 hr at 105° C. Pretreated a StratoSpheres PL-Thiol MP SPE column with methanol, filtered reaction mixture through column, washed with Methanol. Concentrated to give the crude product, which was dissolved in DMSO, and purified on a Gilson HPLC (XBridge 19×100 mm 5u preparatory column), eluting at 18 mL/min with a linear gradient running from 20% to 95% acetonitrile and 0.1% aqueous NH₄OH over 18 min. The desired fractions were concentrated under a stream of nitrogen at 45° C., giving the desired product (8.75 mg, 31.5%).

LC/MS: m/z=465.0[M+H]⁺, Ret. Time: 1.08 min.

### Example 112

### 1-methylethyl 2-{4-[(3-{[2-(trifluoromethyl)phenyl]amino}phenyl)methyl]-1-piperazinyl}-3-pyridinecarboxylate

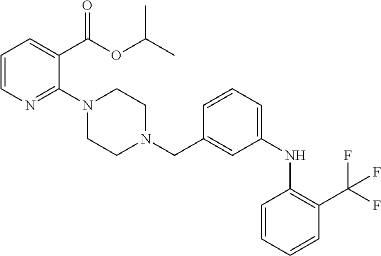 [SMILES: CC(C)OC(=O)c1cccnc1N1CCN(Cc2cccc(Nc3ccccc3C(F)(F)F)c2)CC1]

Added the 1-methylethyl 2-{4-[(3-bromophenyl)methyl]-1-piperazinyl}-3-pyridinecarboxylate (25.0 mg, 0.060 mmol), [2-(trifluoromethyl)phenyl]amine (0.120 mmol), XPhos (5.7 mg, 0.012 mmol), potassium phosphate (38.1 mg, 0.179 mmol) and palladium(II) acetate (1.34 mg, 10 mol %) in 3.0 mL Toluene in a 5 mL reaction vial, and then stirred the mixture for 30 min at room temperature under nitrogen atmosphere. The stirring was kept for 12 hr at 105° C. Pretreated a StratoSpheres PL-Thiol MP SPE column with methanol, filtered reaction mixture through column, washed with Methanol. Concentrated to give the crude product, which was dissolved in DMSO, and purified on a Gilson HPLC (XBridge 19×100 mm 5u preparatory column), eluting at 18 mL/min with a linear gradient running from 20% to 95% acetonitrile and 0.1% aqueous NH₄OH over 18 min. The desired fractions were concentrated under a stream of nitrogen at 45° C., giving the desired product (11.48 mg, 38.5%). LC/MS: m/z=499.0[M+H]⁺, Ret. Time: 1.07 min.

Following the procedure as described above in the preparation of 1-methylethyl 2-{4-[(3-{[2-(trifluoromethyl)phenyl]amino}phenyl)methyl]-1-piperazinyl}-3-pyridinecarboxylate, aryl anilines (0.12 mmol) was reacted with the appropriate boronic acid to yield the examples listed in Table V.

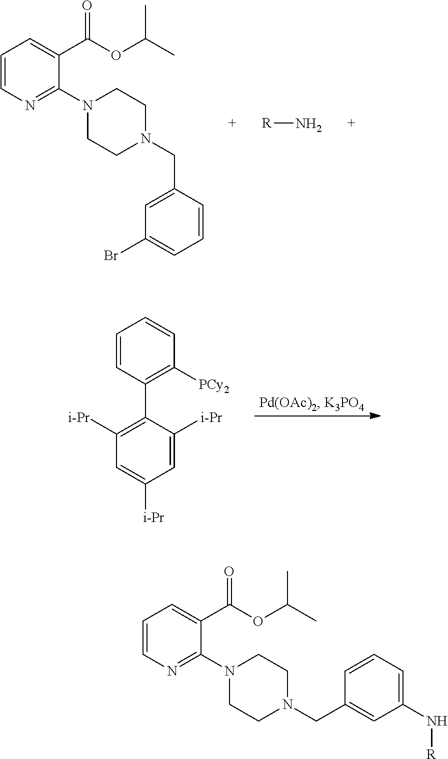

TABLE V

| | | | LC-MS | |
|---|---|---|---|---|
| | | | m/z | RT |
| Example | Aniline | Product | (M + H)⁺ | (min) |
| Example 113 | 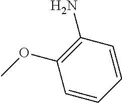 | 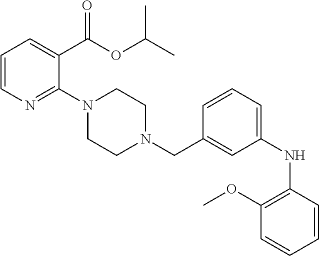 [SMILES: COc1ccccc1Nc1cccc(CN2CCN(c3ncccc3C(=O)OC(C)C)CC2)c1] 1-methylethyl 2-{4-[(3-{[2- (methyloxy)phenyl]amino} phenyl)methyl]-1-piperazinyl}- 3-pyridinecarboxylate | 461 | 1.0 |
| Example 114 | 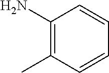 | 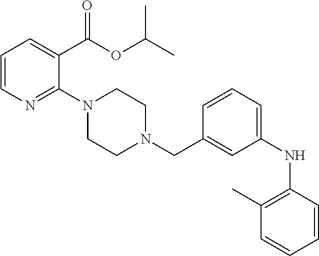 [SMILES: Cc1ccccc1Nc1cccc(CN2CCN(c3ncccc3C(=O)OC(C)C)CC2)c1] 1-methylethyl 2-[4-({3-[(2- methylphenyl)amino]phenyl} methyl)-1-piperazinyl]-3- pyridinecarboxylate | 445 | 1.1 |
| Example 115 | 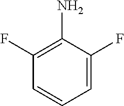 | 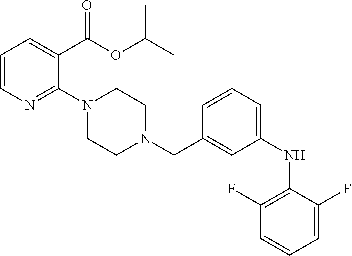 [SMILES: CC(C)OC(=O)c1cccnc1N1CCN(Cc2cccc(Nc3c(F)cccc3F)c2)CC1] 1-methylethyl 2-[4-({3-[(2,6- difluorophenyl)amino] phenyl}methyl)-1-piperazinyl]-3- pyridinecarboxylate | 467 | 1.0 |
| Example 116 | 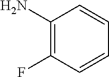 | 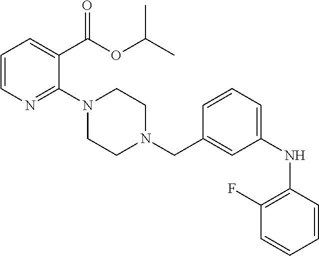 [SMILES: CC(C)OC(=O)c1cccnc1N1CCN(Cc2cccc(Nc3ccccc3F)c2)CC1] 1-methylethyl 2-[4-({3-[(2- fluorophenyl)amino]phenyl} methyl)-1-piperazinyl]-3- pyridinecarboxylate | 449 | 1.0 |
| Example 117 | 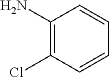 | 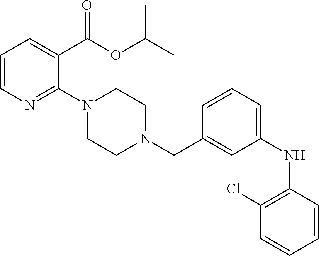 [SMILES: CC(C)OC(=O)c1cccnc1N1CCN(Cc2cccc(Nc3ccccc3Cl)c2)CC1] 1-methylethyl 2-[4-({3-[(2- chlorophenyl)amino]phenyl} methyl)-1-piperazinyl]-3- pyridinecarboxylate | 465 | 1.1 |

### Table 6

### Example 118

### 1-methylethyl 2-(4-{[4-({2-[(trifluoromethyl)oxy]phenyl}amino)phenyl]methyl}-1-piperazinyl)-3-pyridinecarboxylate

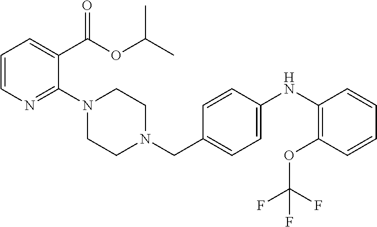 [SMILES: CC(C)OC(=O)c1cccnc1N1CCN(Cc2ccc(Nc3ccccc3OC(F)(F)F)cc2)CC1]

Added the 1-methylethyl 2-{4-[(4-bromophenyl)methyl]-1-piperazinyl}-3-pyridine carboxylate (25.0 mg, 0.060 mmol), Anilines (0.120 mmol), Xphos (5.7 mg, 0.012 mmol), potassium phosphate (38.1 mg, 0.179 mmol) and palladium(II) acetate (1.34 mg, 10 mol %) in 3.0 mL Toluene in a 5 mL reaction vial, and then stirred the mixture for 30 min at room temperature under nitrogen atmosphere. The stirring was kept for 12 hr at 105° C. Pretreated a StratoSpheres PL-Thiol MP SPE column with methanol, filtered reaction mixture through column, washed with Methanol. Concentrated to give the crude product, which was dissolved in DMSO, and purified on a Gilson HPLC (XBridge 19×100 mm 5u preparatory column), eluting at 18 mL/min with a linear gradient running from 20% to 95% acetonitrile and 0.1% aqueous NH₄OH over 18 min. The desired fractions were concentrated under a stream of nitrogen at 45° C., giving the desired product (12.83 mg, 41.7%).

LC/MS: m/z=515.0[M+H]⁺, Ret. Time: 1.16 min.

Following the procedure as described above in the preparation of 1-methylethyl 2-(4-{[4-({2-[(trifluoromethyl)oxy]phenyl}amino)phenyl]methyl}-1-piperazinyl)-3-pyridine carboxylate,

1-methylethyl 2-{4-[(4-bromophenyl)methyl]-1-piperazinyl}-3-pyridine carboxylate (25.0 mg, 0.060 mmol) was reacted with the appropriate aryl anilines (0.12 mmol) to yield the examples listed in Table VI.

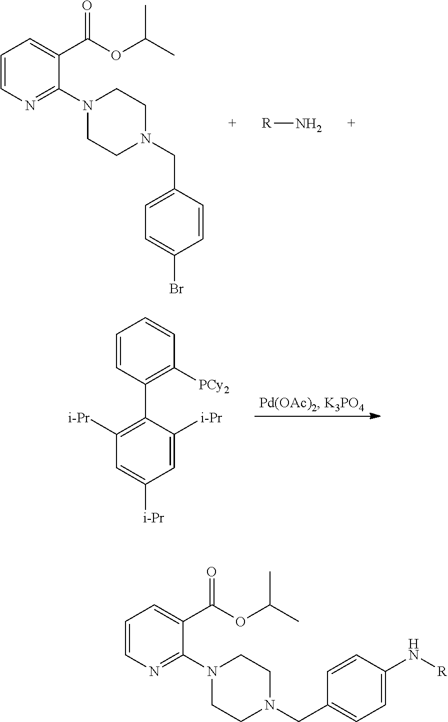

TABLE VI

| | | | LC-MS | |
|---|---|---|---|---|
| | Aryl | | m/z | RT |
| Example | aniline | Product | (M + H)⁺ | (min) |
| Example 119 | 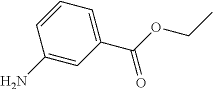 | 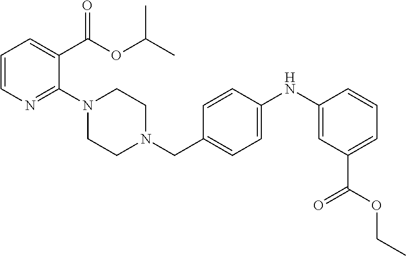 [SMILES: CCOC(=O)c1cccc(Nc2ccc(CN3CCN(c4ncccc4C(=O)OC(C)C)CC3)cc2)c1] 1-methylethyl 2-(4-{[4-({3- (ethyloxy)carbonyl]phenyl} amino)phenyl]methyl}-1- piperazinyl)-3-pyridine carboxylate | 503 | 1.1 |
| Example 120 | 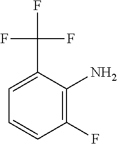 [SMILES: Nc1c(F)cccc1C(F)(F)F] | 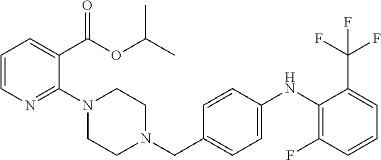 [SMILES: CC(C)OC(=O)c1cccnc1N1CCN(Cc2ccc(Nc3c(F)cccc3C(F)(F)F)cc2)CC1] 1-methylethyl 2-{4-[(4-{[2- fluoro-6-(trifluoromethyl) phenyl]amino}phenyl) methyl]-1-piperazinyl}-3- pyridinecarboxylate | 516.9 | 1.1 |
| Example 121 | 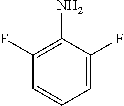 | 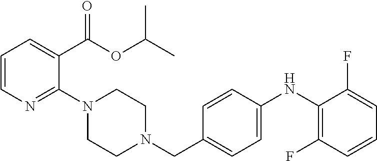 [SMILES: CC(C)OC(=O)c1cccnc1N1CCN(Cc2ccc(Nc3c(F)cccc3F)cc2)CC1] 1-methylethyl 2-[4-({4-[(2,6- difluorophenyl)amino] phenyl}methyl)-1-piperazinyl]-3- pyridinecarboxylate | 467.0 | 1.1 |
| Example 122 | 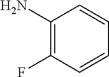 | 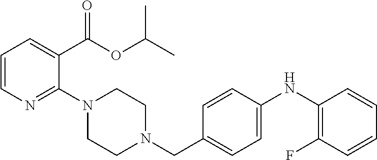 [SMILES: CC(C)OC(=O)c1cccnc1N1CCN(Cc2ccc(Nc3ccccc3F)cc2)CC1] 1-methylethyl 2-[4-({4-[(2- fluorophenyl)amino]phenyl} methyl)-1-piperazinyl]-3- pyridinecarboxylate | 449.0 | 1.1 |
| Example 123 | 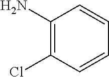 | 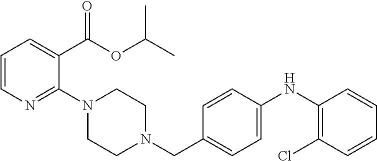 [SMILES: CC(C)OC(=O)c1cccnc1N1CCN(Cc2ccc(Nc3ccccc3Cl)cc2)CC1] 1-methylethyl 2-[4-({4-[(2- chlorophenyl)amino]phenyl} methyl)-1-piperazinyl]-3- pyridinecarboxylate | 465 | 1.1 |
| Example 124 | 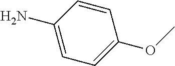 | 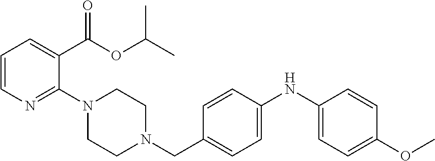 [SMILES: COc1ccc(Nc2ccc(CN3CCN(c4ncccc4C(=O)OC(C)C)CC3)cc2)cc1] 1-methylethyl 2-{4-[(4-{[4- (methyloxy)phenyl]amino} phenyl)methyl]-1- piperazinyl}-3- pyridinecarboxylate | 461.1 | 1.0 |

### Table 7

### Example 125

### 1-methylethyl 2-[4-(2-furanylmethyl)-1-piperazinyl]-4-phenyl-3-pyridinecarboxylate

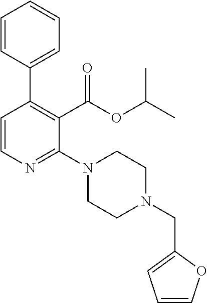 [SMILES: CC(C)OC(=O)c1c(-c2ccccc2)ccnc1N1CCN(Cc2ccco2)CC1]

Dissolved 1-methylethyl 4-phenyl-2-(1-piperazinyl)-3-pyridinecarboxylate (30.0 mg, 0.092 mmol) and furan-2-carbaldehyde (0.24 mmol) in THF (2.5 mL) and DMSO (0.5 mL) with acetic acid (5.54 mg, 0.092 mmol). The solution was stirred for 1 h at room temperature. Then added MP-cyanoborohydride (0.277 mmol) and stirred at room temperature for 12 hr. The polymer was filtered and got the crude product, which was dissolved in DMSO, and purified on a Gilson HPLC (XBridge 19×100 mm 5u preparatory column), eluting at 18 mL/min with a linear gradient running from 30% to 95% acetonitrile and 0.1% aqueous NH₄OH over 18 min. The desired fractions were concentrated under a stream of nitrogen at 45° C., giving the desired product (3.66 mg, 9.79%). LC/MS: m/z=406.1[M+H]⁺, Ret. Time: 0.97 min.

Following the procedure as described above in the preparation of 1-methylethyl 2-[4-(2-furanyl methyl)-1-piperazinyl]-4-phenyl-3-pyridinecarboxylate, 1-methylethyl 4-phenyl-2-(1-piperazinyl)-3-pyridinecarboxylate (30.0 mg, 0.092 mmol) was reacted with the appropriate aldehydes (0.24 mmol) to yield the examples listed in Table VII.

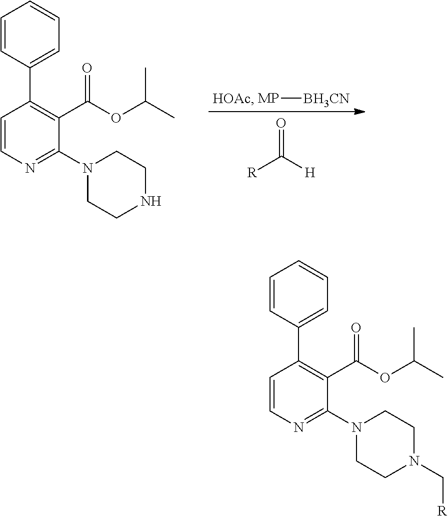

TABLE VII

| | | | LC-MS | |
|---|---|---|---|---|
| Ex- | | | m/z | |
| am- | | | (M + | RT |
| ple | Aldehyde | Product | H)⁺ | (min) |
| Ex- am- ple 126 | 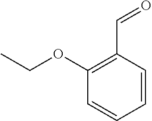 | 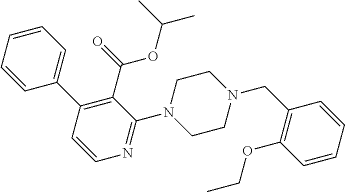 [SMILES: CCOc1ccccc1CN1CCN(c2nccc(-c3ccccc3)c2C(=O)OC(C)C)CC1] 1-methylethyl 2-(4-{[2- (ethyloxy)phenyl]methyl}- 1-piperazinyl)-4-phenyl- 3-pyridinecarboxylate | 460.0 | 1.2 |
| Ex- am- ple 127 | 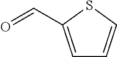 | 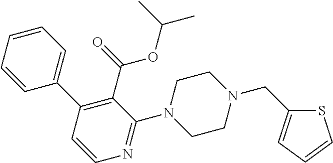 [SMILES: CC(C)OC(=O)c1c(-c2ccccc2)ccnc1N1CCN(Cc2cccs2)CC1] 1-methylethyl 4-phenyl-2- [4-(2-thienylmethyl)-1- piperazinyl]-3- pyridinecarboxylate | 422.0 | 1.0 |
| Ex- am- ple 128 | 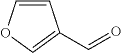 | 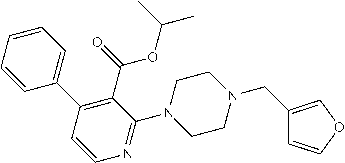 [SMILES: CC(C)OC(=O)c1c(-c2ccccc2)ccnc1N1CCN(Cc2ccoc2)CC1] 1-methylethyl 2-[4-(3- furanylmethyl)-1- piperazinyl]-4-phenyl-3- pyridinecarboxylate | 406.0 | 1.0 |
| Ex- am- ple 129 | 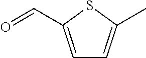 | 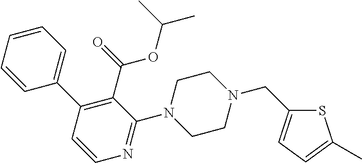 [SMILES: Cc1ccc(CN2CCN(c3nccc(-c4ccccc4)c3C(=O)OC(C)C)CC2)s1] 1-methylethyl 2-{4-[(5- methyl-2-thienyl)methyl]- 1-piperazinyl}-4-phenyl- 3-pyridinecarboxylate | 436.0 | 1.0 |
| Ex- am- ple 130 | 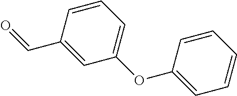 [SMILES: O=Cc1cccc(Oc2ccccc2)c1] | 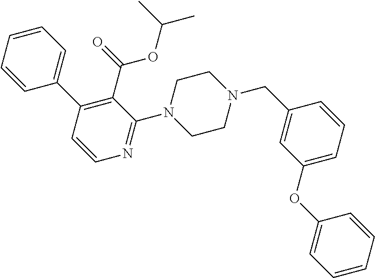 [SMILES: CC(C)OC(=O)c1c(-c2ccccc2)ccnc1N1CCN(Cc2cccc(Oc3ccccc3)c2)CC1] 1-methylethyl 4-phenyl-2- (4-{[3-(phenyloxy) phenyl]methyl}- 1-piperazinyl)-3- pyridinecarboxylate | 508.0 | 1.2 |
| Ex- am- ple 131 | 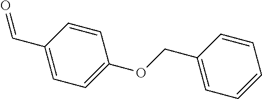 [SMILES: O=Cc1ccc(OCc2ccccc2)cc1] | 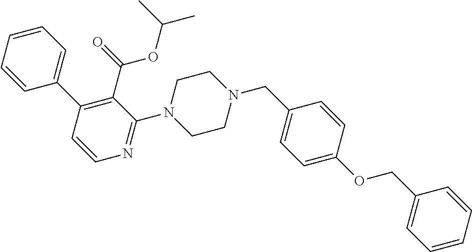 [SMILES: CC(C)OC(=O)c1c(-c2ccccc2)ccnc1N1CCN(Cc2ccc(OCc3ccccc3)cc2)CC1] 1-methylethyl 4-phenyl-2- (4-{[3- (phenyloxy)phenyl]methyl}- 1-piperazinyl)-3- pyridinecarboxylate | 522.0 | 1.2 |
| Ex- am- ple 132 | 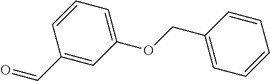 [SMILES: O=Cc1cccc(OCc2ccccc2)c1] | 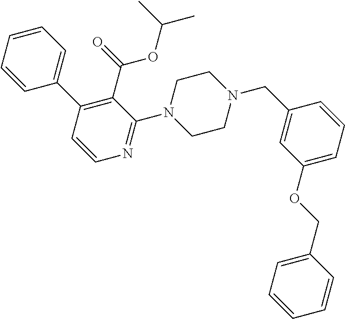 [SMILES: CC(C)OC(=O)c1c(-c2ccccc2)ccnc1N1CCN(Cc2cccc(OCc3ccccc3)c2)CC1] 1-methylethyl 4-phenyl-2- [4-({3- [(phenylmethyl)oxy] phenyl}methyl)-1-piperazinyl]- 3-pyridinecarboxylate | 522.0 | 1.2 |
| Ex- am- ple 133 | 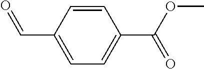 | 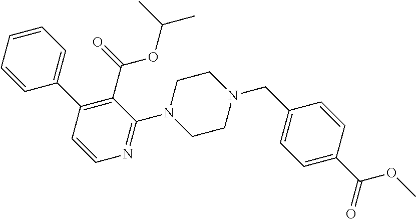 [SMILES: COC(=O)c1ccc(CN2CCN(c3nccc(-c4ccccc4)c3C(=O)OC(C)C)CC2)cc1] 1-methylethyl 4-phenyl-2- [4-({3- [(phenylmethyl)oxy] phenyl}methyl)-1-piperazinyl]- 3-pyridinecarboxylate | 474.0 | 1.0 |
| Ex- am- ple 134 | 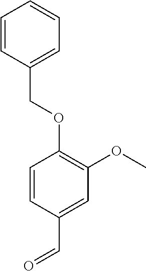 [SMILES: COc1cc(C=O)ccc1OCc1ccccc1] | 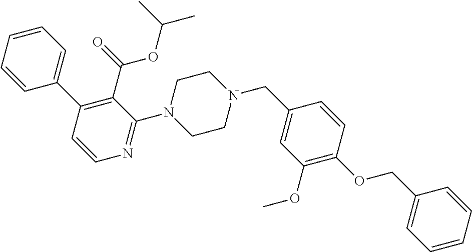 [SMILES: COc1cc(CN2CCN(c3nccc(-c4ccccc4)c3C(=O)OC(C)C)CC2)ccc1OCc1ccccc1] 1-methylethyl 2-[4-({3- (methyloxy)-4- [(phenylmethyl)oxy] phenyl}methyl)-1-piperazinyl]- 4-phenyl-3- pyridinecarboxylate | 552.0 | 1.2 |
| Ex- am- ple 135 | 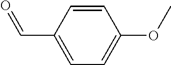 | 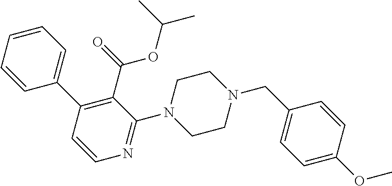 [SMILES: COc1ccc(CN2CCN(c3nccc(-c4ccccc4)c3C(=O)OC(C)C)CC2)cc1] 1-methylethyl 2-[4-({3- (methyloxy)-4- [(phenylmethyl)oxy] phenyl}methyl)-1-piperazinyl]- 4-phenyl-3- pyridinecarboxylate | 446.1 | 1.0 |
| Ex- am- ple 136 | 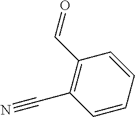 | 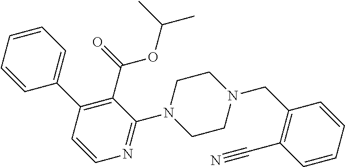 [SMILES: CC(C)OC(=O)c1c(-c2ccccc2)ccnc1N1CCN(Cc2ccccc2C#N)CC1] 1-methylethyl 2-{4-[(2- cyanophenyl)methyl]-1- piperazinyl}-4-phenyl-3- pyridinecarboxylate | 441.0 | 1.0 |
| Ex- am- ple 137 | 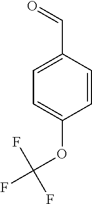 [SMILES: O=Cc1ccc(OC(F)(F)F)cc1] | 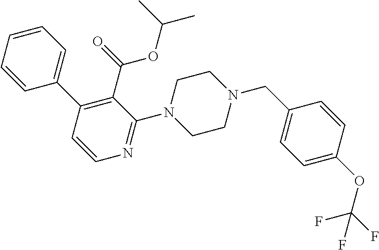 [SMILES: CC(C)OC(=O)c1c(-c2ccccc2)ccnc1N1CCN(Cc2ccc(OC(F)(F)F)cc2)CC1] 1-methylethyl 4-phenyl-2- [4-({4-[(trifluoromethyl) oxy]phenyl}methyl)-1- piperazinyl]-3- pyridinecarboxylate | 500.0 | 1.1 |
| Ex- am- ple 138 | 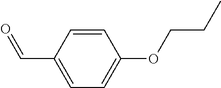 | 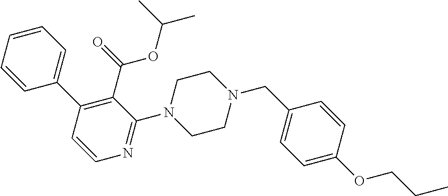 [SMILES: CCCOc1ccc(CN2CCN(c3nccc(-c4ccccc4)c3C(=O)OC(C)C)CC2)cc1] 1-methylethyl 4-phenyl-2- (4-{[4- (propyloxy)phenyl]methyl}- 1-piperazinyl)-3- pyridinecarboxylate | 474.0 | 1.1 |
| Ex- am- ple 139 | 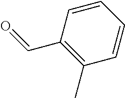 | 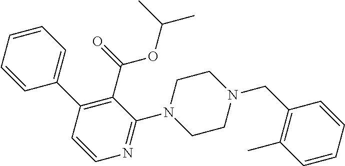 [SMILES: Cc1ccccc1CN1CCN(c2nccc(-c3ccccc3)c2C(=O)OC(C)C)CC1] 1-methylethyl 2-{4-[(2- methylphenyl)methyl]-1- piperazinyl}-4-phenyl-3- pyridinecarboxylate | 430.0 | 1.0 |
| Ex- am- ple 140 | 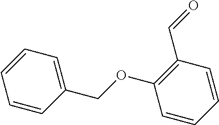 [SMILES: O=Cc1ccccc1OCc1ccccc1] | 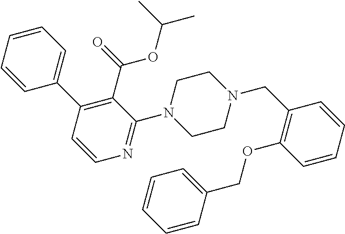 [SMILES: CC(C)OC(=O)c1c(-c2ccccc2)ccnc1N1CCN(Cc2ccccc2OCc2ccccc2)CC1] 1-methylethyl 4-phenyl-2- [4-({2- [(phenylmethyl)oxy]phenyl} methyl)-1-piperazinyl]- 3-pyridinecarboxylate | 522.0 | 1.2 |
| Ex- am- ple 141 | 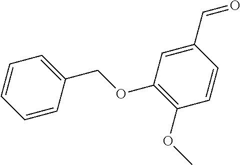 [SMILES: COc1ccc(C=O)cc1OCc1ccccc1] | 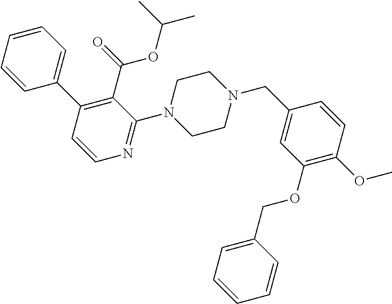 [SMILES: COc1ccc(CN2CCN(c3nccc(-c4ccccc4)c3C(=O)OC(C)C)CC2)cc1OCc1ccccc1] 1-methylethyl 2-[4-({4- (methyloxy)-3- [(phenylmethyl)oxy] phenyl}methyl)-1-piperazinyl]- 4-phenyl-3- pyridinecarboxylate | 552.0 | 1.2 |
| Ex- am- ple 142 | 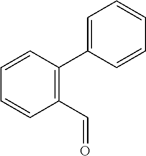 [SMILES: O=Cc1ccccc1-c1ccccc1] | 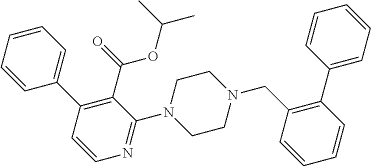 [SMILES: CC(C)OC(=O)c1c(-c2ccccc2)ccnc1N1CCN(Cc2ccccc2-c2ccccc2)CC1] 1-methylethyl 2-[4-(2- biphenylylmethyl)-1- piperazinyl]-4-phenyl-3- pyridinecarboxylate | 492.0 | 1.2 |
| Ex- am- ple 143 | 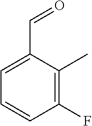 | 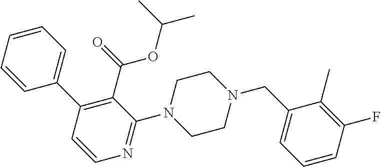 [SMILES: Cc1c(F)cccc1CN1CCN(c2nccc(-c3ccccc3)c2C(=O)OC(C)C)CC1] 1-methylethyl 2-{4-[(3- fluoro-2- methylphenyl)methyl]-1- piperazinyl}-4-phenyl-3- pyridinecarboxylate | 448.1 | 1.1 |
| Ex- am- ple 144 | 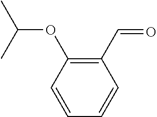 | 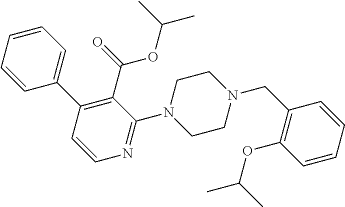 [SMILES: CC(C)OC(=O)c1c(-c2ccccc2)ccnc1N1CCN(Cc2ccccc2OC(C)C)CC1] 1-methylethyl 2-[4-({2-[(1- methylethyl)oxy]phenyl} methyl)-1-piperazinyl]-4- phenyl-3- pyridinecarboxylate | 474.1 | 1.1 |
| Ex- am- ple 145 | 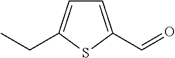 | 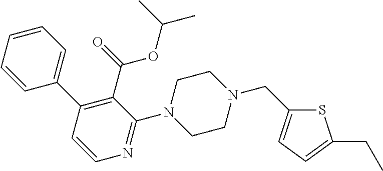 [SMILES: CCc1ccc(CN2CCN(c3nccc(-c4ccccc4)c3C(=O)OC(C)C)CC2)s1] 1-methylethyl 2-[4-({2-[(1- methylethyl)oxy]phenyl} methyl)-1-piperazinyl]-4- phenyl-3- pyridinecarboxylate | 450.0 | 1.1 |
| Ex- am- ple 146 | 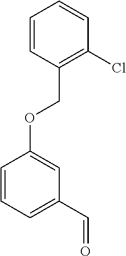 [SMILES: O=Cc1cccc(OCc2ccccc2Cl)c1] | 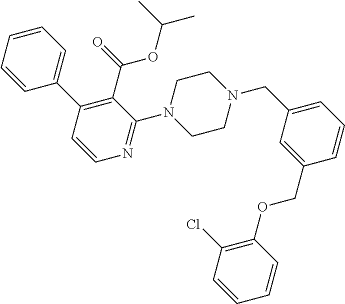 [SMILES: CC(C)OC(=O)c1c(-c2ccccc2)ccnc1N1CCN(Cc2cccc(COc3ccccc3Cl)c2)CC1] 1-methylethyl 2-{4-[(3- {[(2- chlorophenyl)methyl]oxy} phenyl)methyl]-1- piperazinyl}-4-phenyl-3- pyridinecarboxylate | 556 | 1.2 |
| Ex- am- ple 147 | 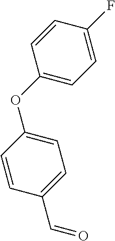 [SMILES: O=Cc1ccc(Oc2ccc(F)cc2)cc1] | 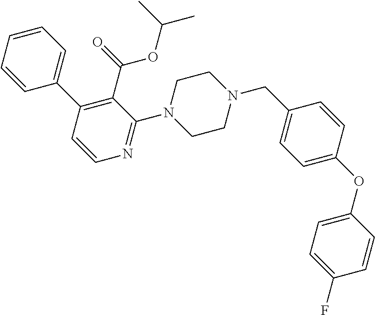 [SMILES: CC(C)OC(=O)c1c(-c2ccccc2)ccnc1N1CCN(Cc2ccc(Oc3ccc(F)cc3)cc2)CC1] 1-methylethyl 2-[4-({4-[(4- fluorophenyl)oxy]phenyl} methyl)-1-piperazinyl]-4- phenyl-3- pyridinecarboxylate | 526.0 | 1.2 |

### Table 8

### Example 148

### 1-methylethyl 2-[(3R)-3-[ethyl(2-furanylmethyl)amino]-1-pyrrolidinyl]-4-phenyl-3-pyridinecarboxylate

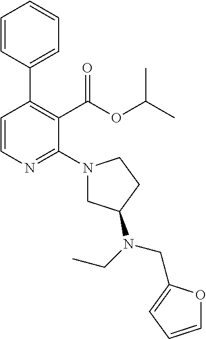 [SMILES: CCN(Cc1ccco1)[C@@H]1CCN(c2nccc(-c3ccccc3)c2C(=O)OC(C)C)C1]

Dissolved 1-methylethyl 2-[(3R)-3-(ethylamino)-1-pyrrolidinyl]-4-phenyl-3-pyridinecarboxylate (25.0 mg, 0.071 mmol) and furan-2-carbaldehyde (0.212 mmol) in methanol (2.5 mL) with acetic acid (1 mg, 0.014 mmol). The solution was stirred for 1 h at room temperature. Then added sodiumcyanoborohydride (15.56 mg, 0.248 mmol) and stirred at room temperature for 12 hr. The polymer was filtered and concentrated the filtrate to give crude product, which was dissolved in DMSO, and purified on a Gilson HPLC (XBridge 19×100 mm 5u preparatory column), eluting at 18 mL/min with a linear gradient running from 30% to 90% acetonitrile and 0.1% aqueous NH₄OH over 15 min. The desired fractions were concentrated under a stream of nitrogen at 45° C., giving the desired product (3.34 mg, 10.89%). LC/MS: m/z=434.1[M+H]⁺, Ret. Time: 0.96 min.

Following the procedure as described above in the preparation of 1-methylethyl 2-{(3R)-3-[ethyl(2-furanyl methyl)amino]-1-pyrrolidinyl}-4-phenyl-3-pyridinecarboxylate, 1-methylethyl 2-[(3R)-3-(ethylamino)-1-pyrrolidinyl]-4-phenyl-3-pyridine carboxylate (25.0 mg, 0.071 mmol) was reacted with the appropriate aldehydes (0.212 mmol) yield the examples listed in Table VIII.

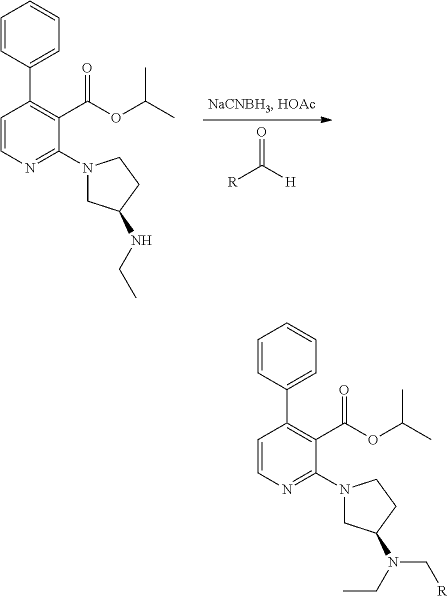

TABLE VIII

| | | | LC-MS | |
|---|---|---|---|---|
| Ex- | | | m/z | |
| am- | | | (M + | RT |
| ple | Aldehyde | Product | H)⁺ | (min) |
| Ex- am- ple 149 | 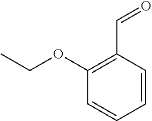 | 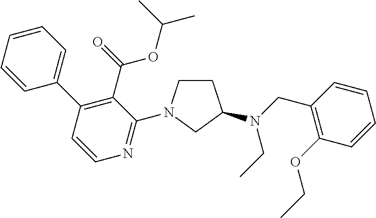 [SMILES: CCOc1ccccc1CN(CC)[C@@H]1CCN(c2nccc(-c3ccccc3)c2C(=O)OC(C)C)C1] 1-methylethyl 2-[(3R)-3- (ethyl{[2- (ethyloxy)phenyl]methyl} amino)-1-pyrrolidinyl]-4- phenyl-3- pyridinecarboxylate | 488.1 | 1.1 |
| Ex- am- ple 150 | 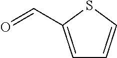 | 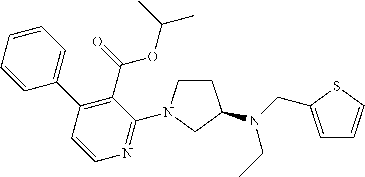 [SMILES: CCN(Cc1cccs1)[C@@H]1CCN(c2nccc(-c3ccccc3)c2C(=O)OC(C)C)C1] 1-methylethyl 2-{(3R)-3- [ethyl(2- thienylmethyl)amino]-1- pyrrolidinyl}-4-phenyl-3- pyridinecarboxylate | 450.0 | 0.99 |
| Ex- am- ple 151 | 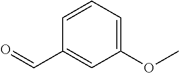 | 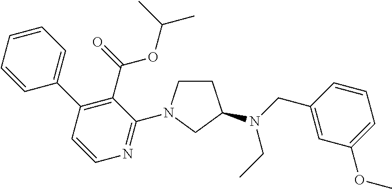 [SMILES: CCN(Cc1cccc(OC)c1)[C@@H]1CCN(c2nccc(-c3ccccc3)c2C(=O)OC(C)C)C1] 1-methylethyl 2-[(3R)-3- (ethyl{[3- (methyloxy)phenyl]methyl} amino)-1-pyrrolidinyl]-4- phenyl-3- pyridinecarboxylate | 474.1 | 1.0 |
| Ex- am- ple 152 | 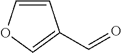 | 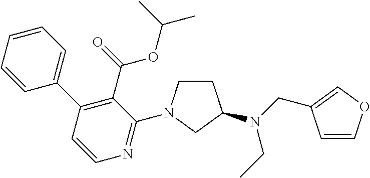 [SMILES: CCN(Cc1ccoc1)[C@@H]1CCN(c2nccc(-c3ccccc3)c2C(=O)OC(C)C)C1] 1-methylethyl 2-{(3R)-3- [ethyl(3- furanylmethyl)amino]-1- pyrrolidinyl}-4-phenyl-3- pyridinecarboxylate | 434.1 | 1.0 |
| Ex- am- ple 153 | 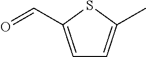 | 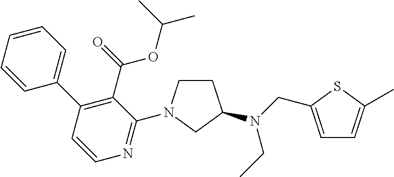 [SMILES: CCN(Cc1ccc(C)s1)[C@@H]1CCN(c2nccc(-c3ccccc3)c2C(=O)OC(C)C)C1] 1-methylethyl 2-((3R)-3- {ethyl[(5-methyl-2- thienyl)methyl]amino}-1- pyrrolidinyl)-4-phenyl-3- pyridinecarboxylate | 464.0 | 1.1 |
| Ex- am- ple 154 | 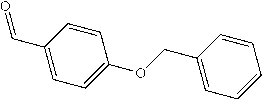 [SMILES: O=Cc1ccc(OCc2ccccc2)cc1] | 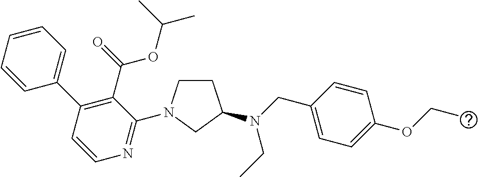 [SMILES: CCOc1ccc(CN(CC)[C@@H]2CCN(c3nccc(-c4ccccc4)c3C(=O)OC(C)C)C2)cc1] 1-methylethyl 2-{(3R)-3- [ethyl({4- [(phenylmethyl)oxy]phenyl} methyl)amino]-1- pyrrolidinyl}-4-phenyl-3- pyridinecarboxylate | 550.1 | 1.2 |
| Ex- am- ple 155 | 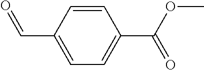 | 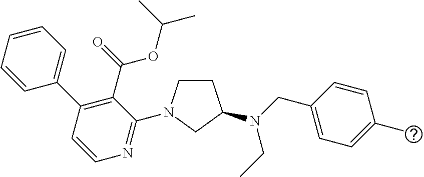 [SMILES: CCN(Cc1ccc(C)cc1)[C@@H]1CCN(c2nccc(-c3ccccc3)c2C(=O)OC(C)C)C1] 1-methylethyl 2-{(3R)-3- [ethyl({4- [(methyloxy)carbonyl] phenyl}methyl)amino]-1- pyrrolidinyl}-4-phenyl-3- pyridinecarboxylate | 502.1 | 1.0 |
| Ex- am- ple 156 | 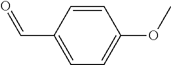 | 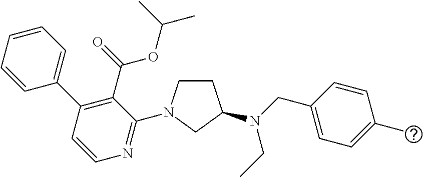 [SMILES: CCN(Cc1ccc(C)cc1)[C@@H]1CCN(c2nccc(-c3ccccc3)c2C(=O)OC(C)C)C1] 1-methylethyl 2-[(3R)-3- (ethyl{[4- (methyloxy)phenyl]methyl} amino)-1-pyrrolidinyl]-4- phenyl-3- pyridinecarboxylate | 474.1 | 1.0 |
| Ex- am- ple 157 | 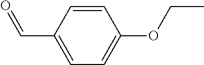 | 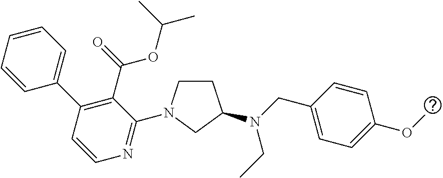 [SMILES: CCN(Cc1ccc(OC)cc1)[C@@H]1CCN(c2nccc(-c3ccccc3)c2C(=O)OC(C)C)C1] 1-methylethyl 2-[(3R)-3- (ethyl{[4- (ethyloxy)phenyl]methyl} amino)-1-pyrrolidinyl]-4- phenyl-3- pyridinecarboxylate | 488.1 | 1.1 |
| Ex- am- ple 158 | 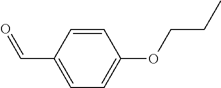 | 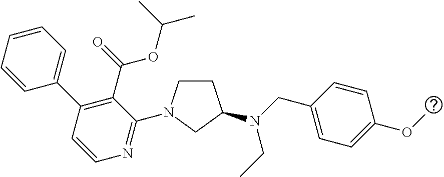 [SMILES: CCN(Cc1ccc(OC)cc1)[C@@H]1CCN(c2nccc(-c3ccccc3)c2C(=O)OC(C)C)C1] 1-methylethyl 2-[(3R)-3- (ethyl{[4- (propyloxy)phenyl]methyl} amino)-1-pyrrolidinyl]-4- phenyl-3- pyridinecarboxylate | 502.1 | 1.2 |
| Ex- am- ple 159 | 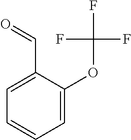 [SMILES: O=Cc1ccccc1OC(F)(F)F] | 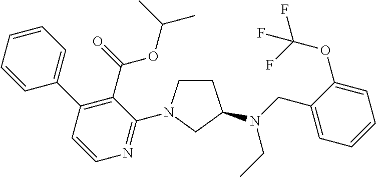 [SMILES: CCN(Cc1ccccc1OC(F)(F)F)[C@@H]1CCN(c2nccc(-c3ccccc3)c2C(=O)OC(C)C)C1] 1-methylethyl 2-{(3R)-3- [ethyl({2- [(trifluoromethyl)oxy] phenyl}methyl)amino]-1- pyrrolidinyl}-4-phenyl-3- pyridinecarboxylate | 528.0 | 1.1 |
| Ex- am- ple 160 | 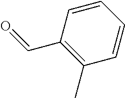 | 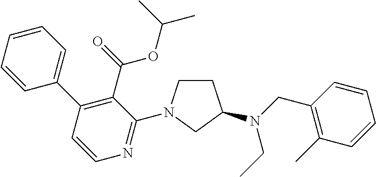 [SMILES: CCN(Cc1ccccc1C)[C@@H]1CCN(c2nccc(-c3ccccc3)c2C(=O)OC(C)C)C1] 1-methylethyl 2-((3R)-3- {ethyl[(2-methylphenyl) methyl]amino}- 1-pyrrolidinyl)-4-phenyl- 3-pyridinecarboxylate | 458.1 | 1.0 |
| Ex- am- ple 161 | 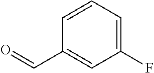 | 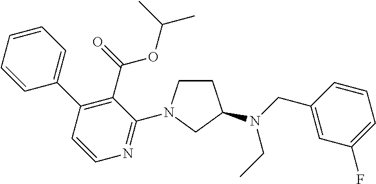 [SMILES: CCN(Cc1cccc(F)c1)[C@@H]1CCN(c2nccc(-c3ccccc3)c2C(=O)OC(C)C)C1] 1-methylethyl 2-((3R)-3- {ethyl[(3-fluorophenyl) methyl]amino}- 1-pyrrolidinyl)-4-phenyl- 3-pyridinecarboxylate | 462.1 | 1.0 |
| Ex- am- ple 162 | 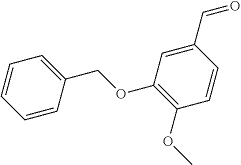 [SMILES: COc1ccc(C=O)cc1OCc1ccccc1] | 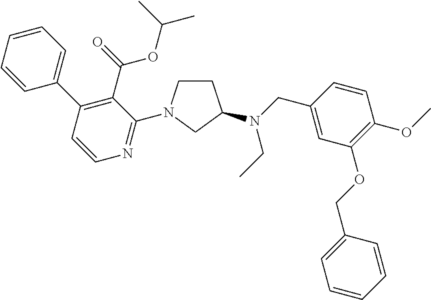 [SMILES: CCN(Cc1ccc(OC)c(OCc2ccccc2)c1)[C@@H]1CCN(c2nccc(-c3ccccc3)c2C(=O)OC(C)C)C1] 1-methylethyl 2-{(3R)-3- [ethyl({4-(methyloxy)-3- [(phenylmethyl)oxy]phenyl} methyl)amino]-1- pyrrolidinyl}-4-phenyl-3- pyridinecarboxylate | 580.1 | 1.2 |
| Ex- am- ple 163 | 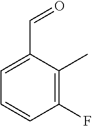 | 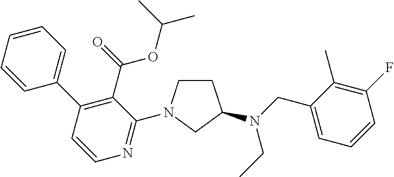 [SMILES: CCN(Cc1cccc(F)c1C)[C@@H]1CCN(c2nccc(-c3ccccc3)c2C(=O)OC(C)C)C1] 1-methylethyl 2-((3R)-3- {ethyl[(3-fluoro-2- methylphenyl)methyl]amino}- 1-pyrrolidinyl)-4-phenyl- 3-pyridinecarboxylate | 476.1 | 1.1 |
| Ex- am- ple 164 | 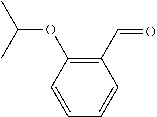 | 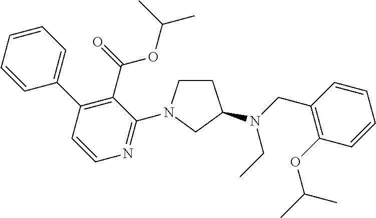 [SMILES: CCN(Cc1ccccc1OC(C)C)[C@@H]1CCN(c2nccc(-c3ccccc3)c2C(=O)OC(C)C)C1] 1-methylethyl 2-{(3R)-3- [ethyl({2-[(1- methylethyl)oxy]phenyl}methyl) amino]-1-pyrrolidinyl}- 4-phenyl-3- pyridinecarboxylate | 502.1 | 1.1 |
| Ex- am- ple 165 | 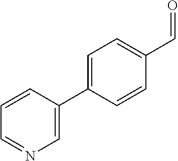 [SMILES: O=Cc1ccc(-c2cccnc2)cc1] | 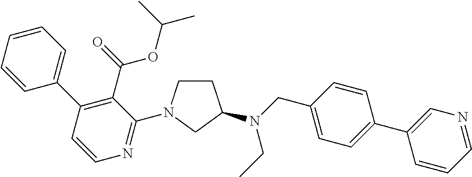 [SMILES: CCN(Cc1ccc(-c2cccnc2)cc1)[C@@H]1CCN(c2nccc(-c3ccccc3)c2C(=O)OC(C)C)C1] 1-methylethyl 2-[(3R)-3- (ethyl{[4-(3- pyridinyl)phenyl]methyl} amino)-1-pyrrolidinyl]-4- phenyl-3- pyridinecarboxylate | 521.1 | 0.9 |
| Ex- am- ple 166 | 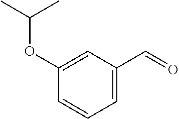 | 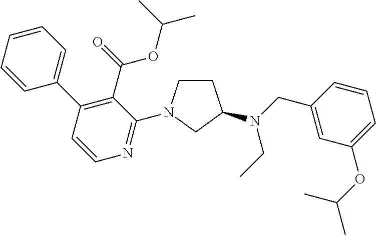 [SMILES: CCN(Cc1cccc(OC(C)C)c1)[C@@H]1CCN(c2nccc(-c3ccccc3)c2C(=O)OC(C)C)C1] 1-methylethyl 2-{(3R)-3- [ethyl({3-[(1- methylethyl)oxy]phenyl} methyl)amino]-1-pyrrolidinyl}- 4-phenyl-3- pyridinecarboxylate | 502.1 | 1.1 |
| Ex- am- ple 167 | 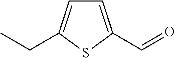 | 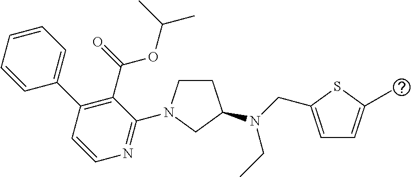 [SMILES: CCN(Cc1ccc(C)s1)[C@@H]1CCN(c2nccc(-c3ccccc3)c2C(=O)OC(C)C)C1] 1-methylethyl 2-((3R)-3- {ethyl[(5-ethyl-2- thienyl)methyl]amino}-1- pyrrolidinyl)-4-phenyl-3- pyridinecarboxylate | 478.0 | 1.1 |
| Ex- am- ple 168 | 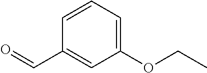 | 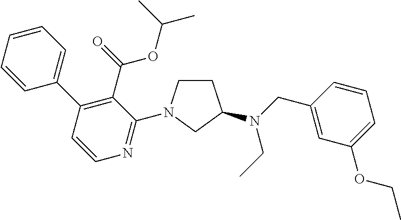 [SMILES: CCOc1cccc(CN(CC)[C@@H]2CCN(c3nccc(-c4ccccc4)c3C(=O)OC(C)C)C2)c1] 1-methylethyl 2-[(3R)-3- (ethyl{[3- (ethyloxy)phenyl]methyl} amino)-1-pyrrolidinyl]-4- phenyl-3- pyridinecarboxylate | 488.1 | 1.1 |

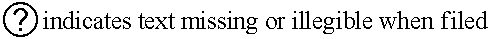

### Table 9

### Example 169

### 1-methylethyl 2-{(3S)-3-[ethyl({4-(methyloxy)-3-[(phenyl methyl)oxy]phenyl}methyl)amino]-1-pyrrolidinyl}-3-pyridine carboxylate

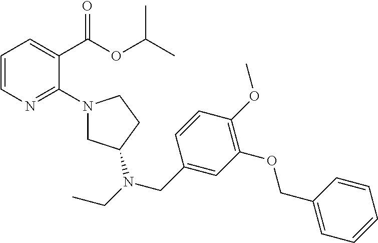 [SMILES: CCN(Cc1ccc(OC)c(OCc2ccccc2)c1)[C@H]1CCN(c2ncccc2C(=O)OC(C)C)C1]

Dissolved 1-methylethyl 2-[(3S)-3-(ethylamino)-1-pyrrolidinyl]-3-pyridine carboxylate (30.0 mg, 0.108 mmol) and furan-2-carbaldehyde (0.27 mmol) in methanol (2.5 mL) with acetic acid (1.3 mg, 0.022 mmol). The solution was stirred for 1 h at room temperature. Then added sodium cyanoborohydride (23.79 mg, 0.379 mmol) and stirred at room temperature for 12 hr. The polymer was filtered and concentrated the filtrate to give crude product, which was dissolved in DMSO, and purified on a Gilson HPLC (XBridge 19×100 mm 5u preparatory column), eluting at 18 mL/min with a linear gradient running from 40% to 95% acetonitrile and 0.1% aqueous NH₄OH over 15 min. The desired fractions were concentrated under a stream of nitrogen at 45° C., giving the desired product (27.4 mg, 50.3%). LC/MS: m/z=504.1[M+H]⁺, Ret. Time: 1.02 min.

Following the procedure as described above in the preparation of 1-methylethyl 2-{(3S)-3-[ethyl({4-(methyloxy)-3-[(phenylmethyl)oxy]phenyl}methyl)amino]-1-pyrrolidinyl}-3-pyridinecarboxylate, 1-methylethyl 2-[(3S)-3-(ethylamino)-1-pyrrolidinyl]-3-pyridine carboxylate (30.0 mg, 0.108 mmol) was reacted with the appropriate aldehydes (0.27 mmol) to yield the examples listed in Table IX.

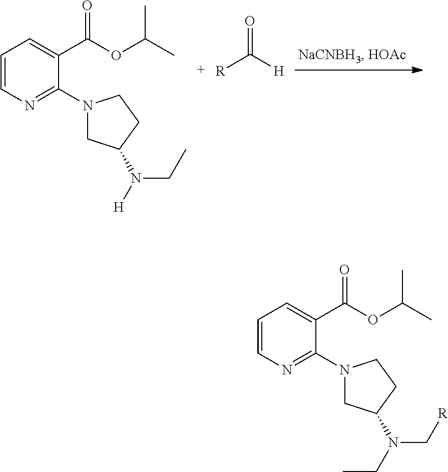

TABLE IX

| | | | LC-MS | |
|---|---|---|---|---|
| | | | m/z | RT |
| Example | Aldehyde | Product | (M + H)⁺ | (min) |
| Example 170 | 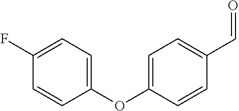 [SMILES: O=Cc1ccc(Oc2ccc(F)cc2)cc1] | 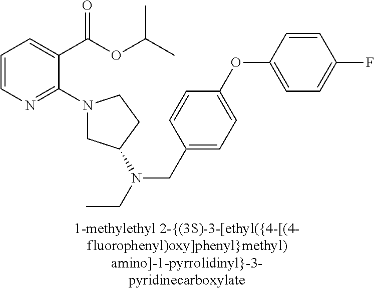 [SMILES: CCN(Cc1ccc(Oc2ccc(F)cc2)cc1)[C@H]1CCN(c2ncccc2C(=O)OC(C)C)C1] | 478.1 | 1.1 |
| Example 171 | 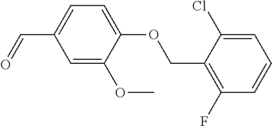 [SMILES: COc1cc(C=O)ccc1OCc1c(F)cccc1Cl] | 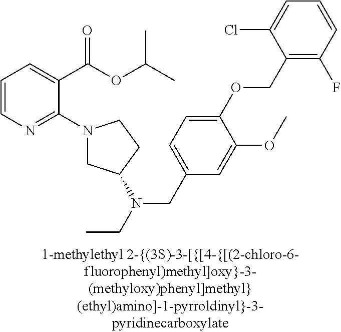 [SMILES: CCN(Cc1ccc(OCc2c(F)cccc2Cl)c(OC)c1)[C@H]1CCN(c2ncccc2C(=O)OC(C)C)C1] | 556.0 | 1.1 |
| Example 172 | 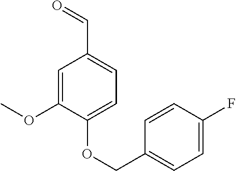 [SMILES: COc1cc(C=O)ccc1OCc1ccc(F)cc1] | 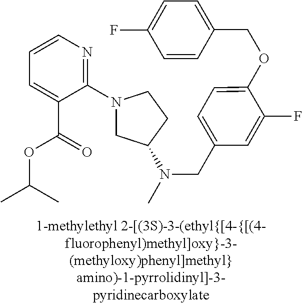 [SMILES: CC(C)OC(=O)c1cccnc1N1CC[C@H](N(C)Cc2ccc(OCc3ccc(F)cc3)c(F)c2)C1] | 522.1 | 1.1 |
| Example 173 | 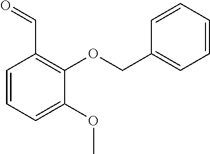 [SMILES: COc1cccc(C=O)c1OCc1ccccc1] | 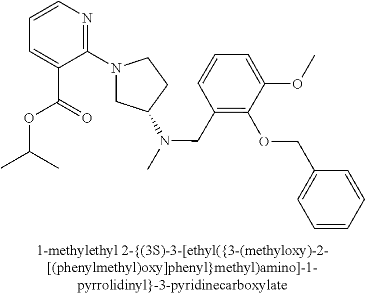 [SMILES: COc1cccc(CN(C)[C@H]2CCN(c3ncccc3C(=O)OC(C)C)C2)c1OCc1ccccc1] | 504.1 | 1.1 |
| Example 174 | 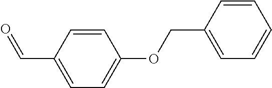 [SMILES: O=Cc1ccc(OCc2ccccc2)cc1] | 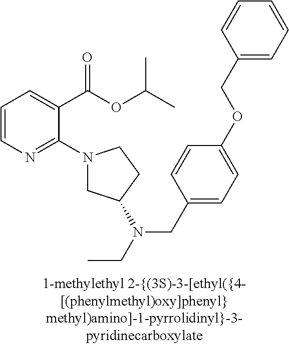 [SMILES: CCN(Cc1ccc(OCc2ccccc2)cc1)[C@H]1CCN(c2ncccc2C(=O)OC(C)C)C1] | 474.1 | 1.1 |
| Example 175 | 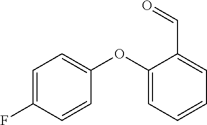 [SMILES: O=Cc1ccccc1Oc1ccc(F)cc1] | 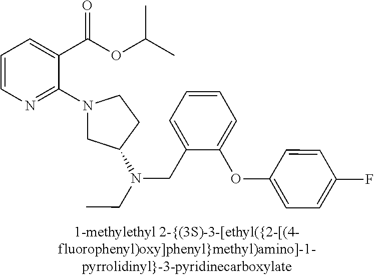 [SMILES: CCN(Cc1ccccc1Oc1ccc(F)cc1)[C@H]1CCN(c2ncccc2C(=O)OC(C)C)C1] | 478.1 | 1.1 |
| Example 176 | 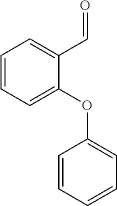 [SMILES: O=Cc1ccccc1Oc1ccccc1] | 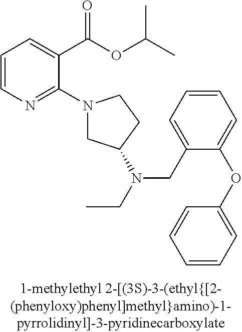 [SMILES: CCN(Cc1ccccc1Oc1ccccc1)[C@H]1CCN(c2ncccc2C(=O)OC(C)C)C1] | 460.1 | 1.1 |
| Example 177 | 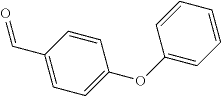 [SMILES: O=Cc1ccc(Oc2ccccc2)cc1] | 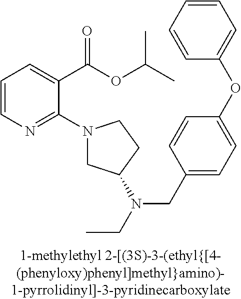 [SMILES: CCN(Cc1ccc(Oc2ccccc2)cc1)[C@H]1CCN(c2ncccc2C(=O)OC(C)C)C1] | 460.1 | 1.1 |
| Example 178 | 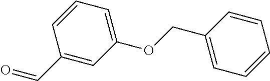 [SMILES: O=Cc1cccc(OCc2ccccc2)c1] | 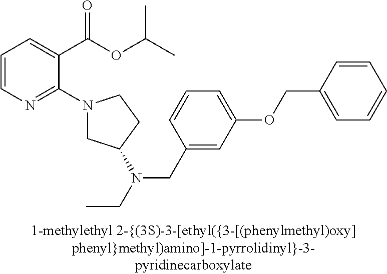 [SMILES: CCN(Cc1cccc(OCc2ccccc2)c1)[C@H]1CCN(c2ncccc2C(=O)OC(C)C)C1] | 474.1 | 1.1 |
| Example 179 | 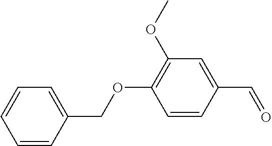 [SMILES: COc1cc(C=O)ccc1OCc1ccccc1] | 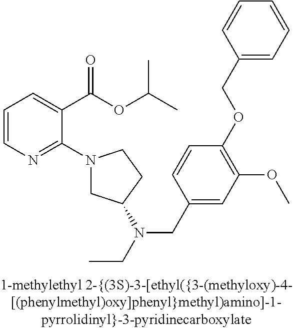 [SMILES: CCN(Cc1ccc(OCc2ccccc2)c(OC)c1)[C@H]1CCN(c2ncccc2C(=O)OC(C)C)C1] | 504.1 | 1.1 |
| Example 180 | 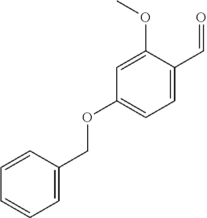 [SMILES: COc1cc(OCc2ccccc2)ccc1C=O] | 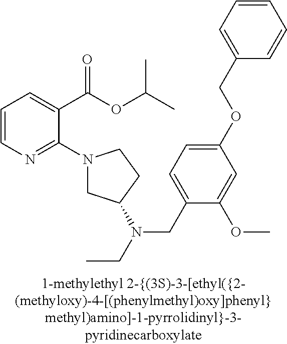 [SMILES: CCN(Cc1ccc(OCc2ccccc2)cc1OC)[C@H]1CCN(c2ncccc2C(=O)OC(C)C)C1] | 504.1 | 1.1 |
| Example 181 | 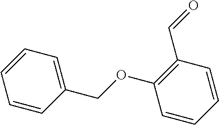 [SMILES: O=Cc1ccccc1OCc1ccccc1] | 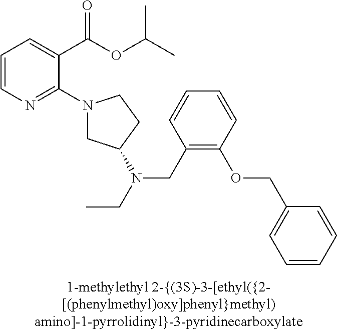 [SMILES: CCN(Cc1ccccc1OCc1ccccc1)[C@H]1CCN(c2ncccc2C(=O)OC(C)C)C1] | 474.1 | 1.1 |
| Example 182 | 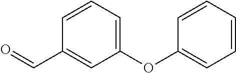 [SMILES: O=Cc1cccc(Oc2ccccc2)c1] | 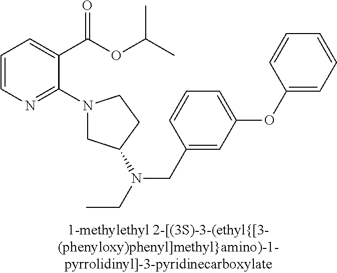 [SMILES: CCN(Cc1cccc(Oc2ccccc2)c1)[C@H]1CCN(c2ncccc2C(=O)OC(C)C)C1] | 460.1 | 1.1 |
| Example 183 | 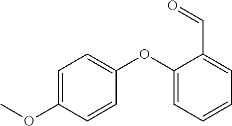 [SMILES: COc1ccc(Oc2ccccc2C=O)cc1] | 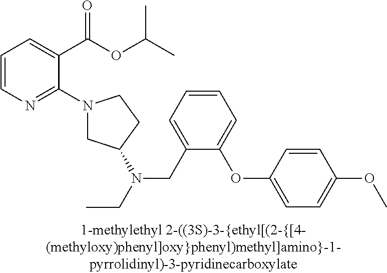 [SMILES: CCN(Cc1ccccc1Oc1ccc(OC)cc1)[C@H]1CCN(c2ncccc2C(=O)OC(C)C)C1] | 490.1 | 1.1 |
| Example 184 | 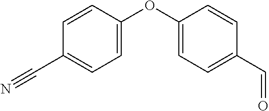 [SMILES: N#Cc1ccc(Oc2ccc(C=O)cc2)cc1] | 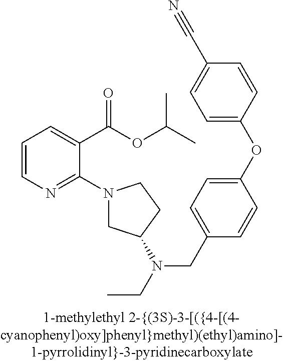 [SMILES: CCN(Cc1ccc(Oc2ccc(C#N)cc2)cc1)[C@H]1CCN(c2ncccc2C(=O)OC(C)C)C1] | 485.1 | 1.0 |
| Example 185 | 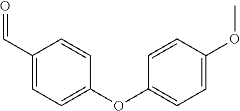 [SMILES: COc1ccc(Oc2ccc(C=O)cc2)cc1] | 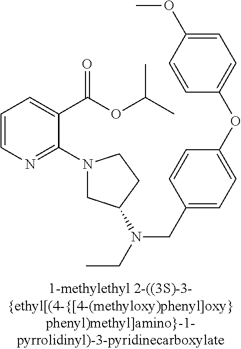 [SMILES: CCN(Cc1ccc(Oc2ccc(OC)cc2)cc1)[C@H]1CCN(c2ncccc2C(=O)OC(C)C)C1] | 490.1 | 1.1 |
| Example 186 | 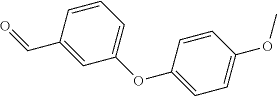 [SMILES: COc1ccc(Oc2cccc(C=O)c2)cc1] | 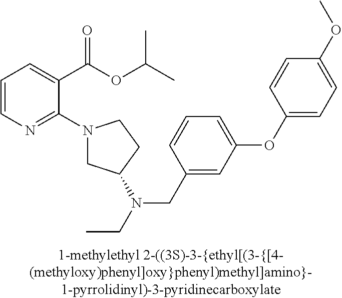 [SMILES: CCN(Cc1cccc(Oc2ccc(OC)cc2)c1)[C@H]1CCN(c2ncccc2C(=O)OC(C)C)C1] | 490.1 | 1.1 |

### Table X

### Example 187

### 1-methylethyl 2-(4-{[4-({[4-(ethyloxy)phenyl]oxy}methyl)phenyl]methyl}-1-piperazinyl)-3-pyridinecarboxylate

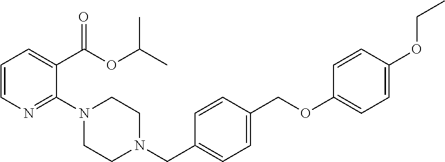 [SMILES: CCOc1ccc(OCc2ccc(CN3CCN(c4ncccc4C(=O)OC(C)C)CC3)cc2)cc1]

To a solution of bis(1-methylethyl) (E)-1,2-diazenedicarboxylate (24.63 mg, 0.122 mmol) in anhydrous THF (1 mL) added Triphenyl phosphine (31.9 mg, 0.122 mmol), the mixture was stirred for 10 min at room temperature. 1-methylethyl 2-(4-{[4-(hydroxymethyl)phenyl]methyl}-1-piperazinyl)-3-pyridinecarboxylate (30 mg, 0.081 mmol) and 4-(ethyloxy)phenol (0.089 mmol) was added to the mixture, and then kept stirring for 12 h at room temperature. Concentrated to give crude product, which was dissolved in DMSO, and purified on a Gilson HPLC (XBridge 19×100 mm 5 u preparatory column), eluting at 18 mL/min with a linear gradient running from 35% to 90% acetonitrile and 0.1% aqueous NH₄OH over 15 min. The desired fractions were concentrated under a stream of nitrogen at 45° C., giving the desired product (4.31 mg, 10.84%). LC/MS: m/z=490.2[M+H]⁺, Ret. Time: 1.10 min.

Following the procedure as described above in the preparation of 1-methylethyl 2-(4-{[4-({[4-(ethyloxy)phenyl]oxy}methyl)phenyl]methyl}-1-piperazinyl)-3-pyridinecarboxylate, 1-methylethyl 2-(4-{[4-(hydroxymethyl)phenyl]methyl}-1-piperazinyl)-3-pyridine carboxylate (30 mg, 0.081 mmol) was reacted with the appropriate phenols (0.089 mmol) to yield the examples listed in Table X.

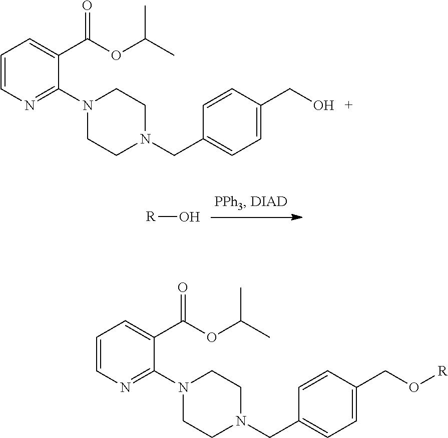

TABLE X

| | | | LC-MS | |
|---|---|---|---|---|
| EX- | | | m/z | |
| AM- | | | (M + | RT |
| PLE | Aldehyde | Product | H)⁺ | (min) |
| Example 188 | 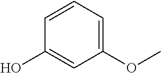 | 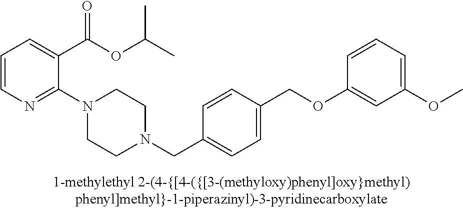 [SMILES: COc1cccc(OCc2ccc(CN3CCN(c4ncccc4C(=O)OC(C)C)CC3)cc2)c1] | 476.2 | 1.1 |
| Example 189 | 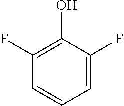 | 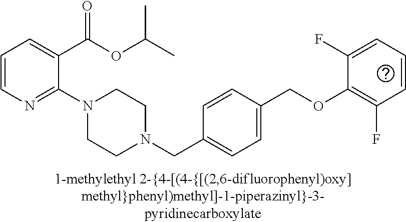 [SMILES: CC(C)OC(=O)c1cccnc1N1CCN(Cc2ccc(COc3c(F)cccc3F)cc2)CC1] | 482.1 | 1.1 |
| Example 190 | 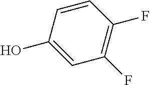 | 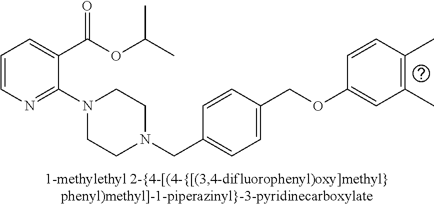 [SMILES: Cc1ccc(OCc2ccc(CN3CCN(c4ncccc4C(=O)OC(C)C)CC3)cc2)cc1C] | 482.1 | 1.1 |
| Example 191 | 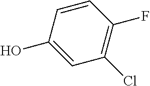 | 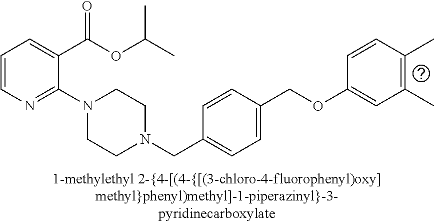 [SMILES: Cc1ccc(OCc2ccc(CN3CCN(c4ncccc4C(=O)OC(C)C)CC3)cc2)cc1C] | 498.1 | 1.2 |
| Example 192 GSK- 2338903A N8941- 55-107 | 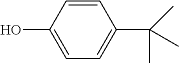 | 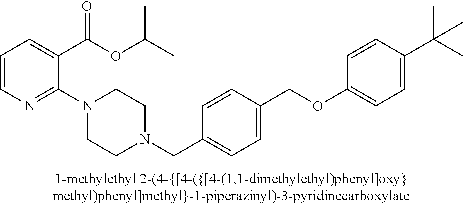 [SMILES: CC(C)OC(=O)c1cccnc1N1CCN(Cc2ccc(COc3ccc(C(C)(C)C)cc3)cc2)CC1] | 502.2 | 1.3 |
| Example 193 | 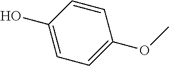 | 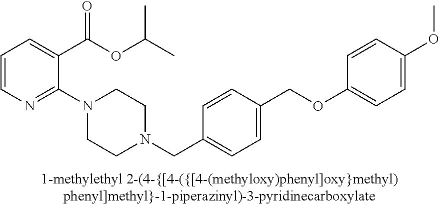 [SMILES: COc1ccc(OCc2ccc(CN3CCN(c4ncccc4C(=O)OC(C)C)CC3)cc2)cc1] | 476.2 | 1.0 |
| Example 194 | 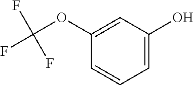 [SMILES: Oc1cccc(OC(F)(F)F)c1] | 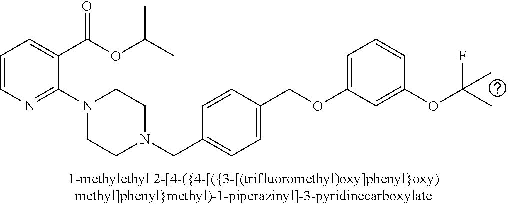 [SMILES: CC(C)OC(=O)c1cccnc1N1CCN(Cc2ccc(COc3cccc(OC(C)(C)F)c3)cc2)CC1] | 530.1 | 1.2 |
| Example 195 | 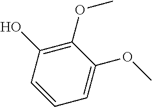 | 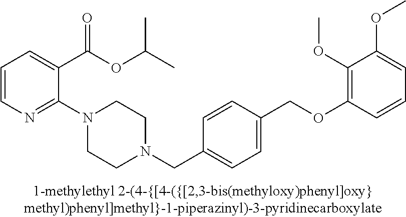 [SMILES: COc1cccc(OCc2ccc(CN3CCN(c4ncccc4C(=O)OC(C)C)CC3)cc2)c1OC] | 506.2 | 1.0 |
| Example 196 | 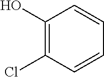 | 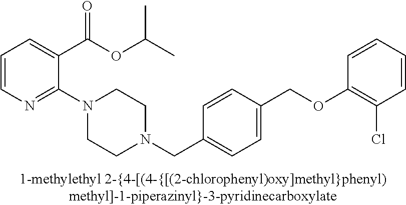 [SMILES: CC(C)OC(=O)c1cccnc1N1CCN(Cc2ccc(COc3ccccc3Cl)cc2)CC1] | 480.1 | 1.1 |
| Example 197 | 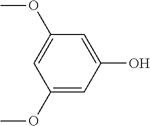 | 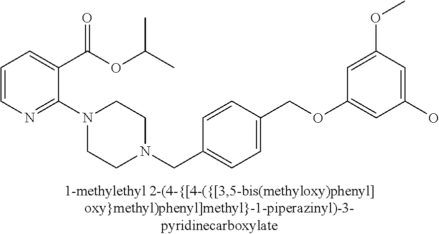 [SMILES: COc1cc(O)cc(OCc2ccc(CN3CCN(c4ncccc4C(=O)OC(C)C)CC3)cc2)c1] | 506.2 | 1.1 |
| Example 198 | 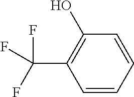 [SMILES: Oc1ccccc1C(F)(F)F] | 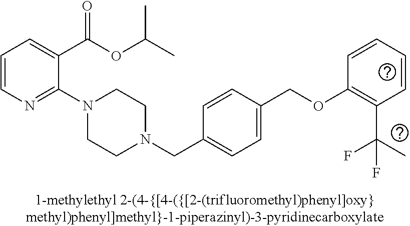 [SMILES: CC(C)OC(=O)c1cccnc1N1CCN(Cc2ccc(COc3ccccc3C(C)(F)F)cc2)CC1] | 514.1 | 1.2 |
| Example 199 | 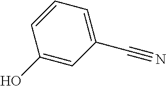 | 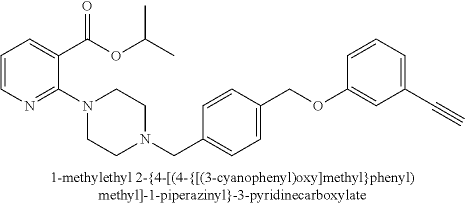 [SMILES: C#Cc1cccc(OCc2ccc(CN3CCN(c4ncccc4C(=O)OC(C)C)CC3)cc2)c1] | 471.2 | 1.0 |
| Example 200 | 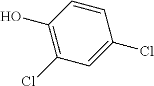 | 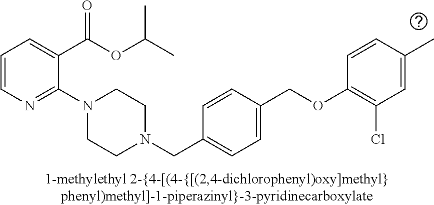 [SMILES: Cc1ccc(OCc2ccc(CN3CCN(c4ncccc4C(=O)OC(C)C)CC3)cc2)c(Cl)c1] | 514.1 | 1.2 |
| Example 201 | 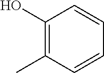 | 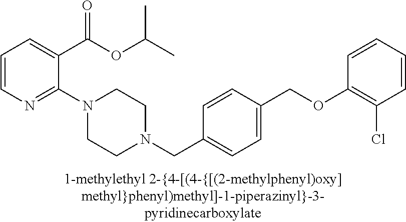 [SMILES: CC(C)OC(=O)c1cccnc1N1CCN(Cc2ccc(COc3ccccc3Cl)cc2)CC1] | 460.2 | 1.1 |
| Example 202 | 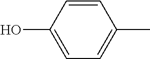 | 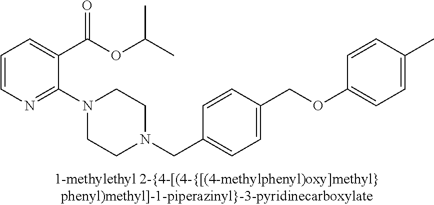 [SMILES: Cc1ccc(OCc2ccc(CN3CCN(c4ncccc4C(=O)OC(C)C)CC3)cc2)cc1] | 460.2 | 1.1 |
| Example 203 | 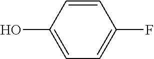 | 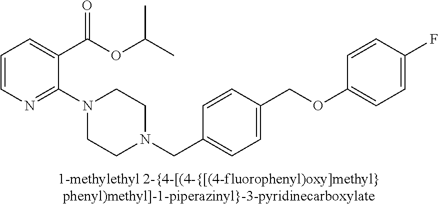 [SMILES: CC(C)OC(=O)c1cccnc1N1CCN(Cc2ccc(COc3ccc(F)cc3)cc2)CC1] | 464.2 | 1.1 |
| Example 204 | 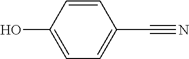 | 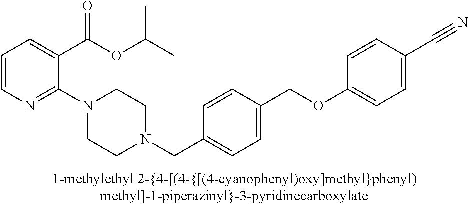 [SMILES: CC(C)OC(=O)c1cccnc1N1CCN(Cc2ccc(COc3ccc(C#N)cc3)cc2)CC1] | 471.2 | 1.0 |

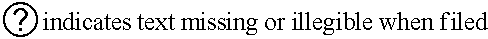

### Table 11

### Example 205

### 1-methylethyl 2-(4-{[3-({[4-(ethyloxy)phenyl]oxy}methyl)phenyl]methyl}-1-piperazinyl)-3-pyridinecarboxylate

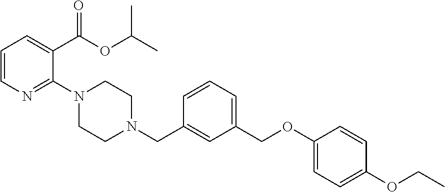 [SMILES: CCOc1ccc(OCc2cccc(CN3CCN(c4ncccc4C(=O)OC(C)C)CC3)c2)cc1]

To a solution of bis(1-methylethyl) (E)-1,2-diazenedicarboxylate (41.0 mg, 0.203 mmol) in anhydrous THF (1 mL) added Triphenyl phosphine (53.2 mg, 0.203 mmol), the mixture was stirred for 10 min at room temperature. 1-methylethyl 2-(4-{[3-(hydroxymethyl)phenyl]methyl}-1-piperazinyl)-3-pyridinecarboxylate (30 mg, 0.081 mmol) and 4-(ethyloxy)phenol (0.081 mmol) was added to the mixture, and then kept stirring for 18 h at room temperature. Concentrated to give crude product, which was dissolved in DMSO, and purified on a Gilson HPLC (XBridge 19×100 mm 5 u preparatory column), eluting at 18 mL/min with a linear gradient running from 40% to 90% acetonitrile and 0.1% aqueous NH₄OH over 15 min. The desired fractions were concentrated under a stream of nitrogen at 45° C., giving the desired product (4.82 mg, 12.12%). LC/MS: m/z=490.1[M+H]⁺, Ret. Time: 1.00 min.

Following the procedure as described above in the preparation of 1-methylethyl 2-(4-{[3-({[4-(ethyloxy)phenyl]oxy}methyl)phenyl]methyl}-1-piperazinyl)-3-pyridine carboxylate, 1-methylethyl 2-(4-{[3-(hydroxymethyl)phenyl]methyl}-1-piperazinyl)-3-pyridine carboxylate (30 mg, 0.081 mmol) was reacted with the appropriate phenols (0.081 mmol) to yield the examples listed in Table XI.

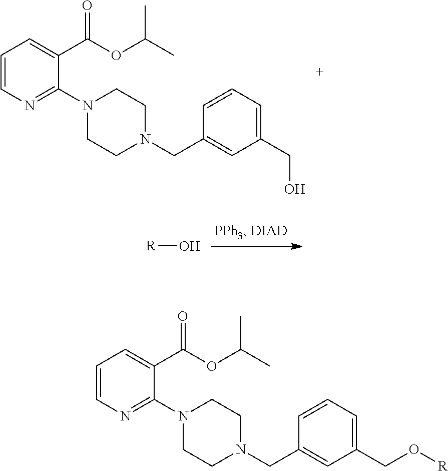

TABLE XI

| | Aldehyde | | LC-MS | |
|---|---|---|---|---|
| | or | | m/z | RT |
| Example | Ketone | Product | (M + H)⁺ | (min) |
| Example 206 | 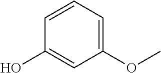 | 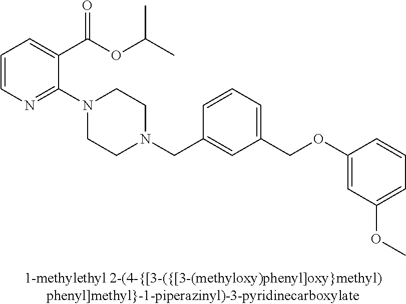 [SMILES: COc1cccc(OCc2cccc(CN3CCN(c4ncccc4C(=O)OC(C)C)CC3)c2)c1] | 476.1 | 1.1 |
| Example 207 | 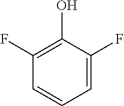 | 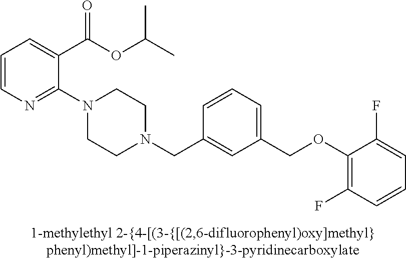 [SMILES: CC(C)OC(=O)c1cccnc1N1CCN(Cc2cccc(COc3c(F)cccc3F)c2)CC1] | 482.1 | 1.0 |
| Example 208 | 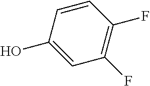 | 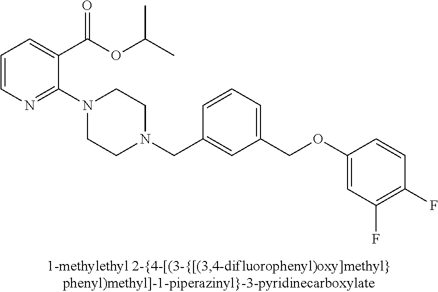 [SMILES: CC(C)OC(=O)c1cccnc1N1CCN(Cc2cccc(COc3ccc(F)c(F)c3)c2)CC1] | 481.2 | 1.0 |
| Example 209 | 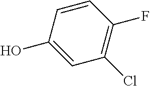 | 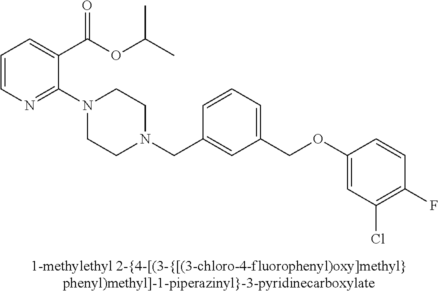 [SMILES: CC(C)OC(=O)c1cccnc1N1CCN(Cc2cccc(COc3ccc(F)c(Cl)c3)c2)CC1] | 498.0 | 1.1 |
| Example 210 | 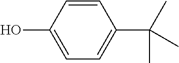 | 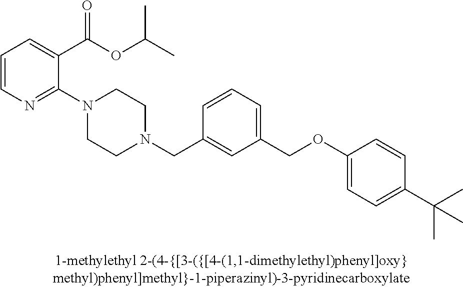 [SMILES: CC(C)OC(=O)c1cccnc1N1CCN(Cc2cccc(COc3ccc(C(C)(C)C)cc3)c2)CC1] | 502.2 | 1.2 |
| Example 211 | 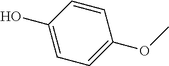 | 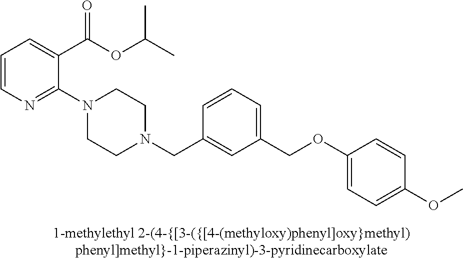 [SMILES: COc1ccc(OCc2cccc(CN3CCN(c4ncccc4C(=O)OC(C)C)CC3)c2)cc1] | 476.1 | 1.0 |
| Example 212 | 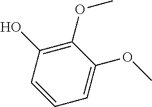 | 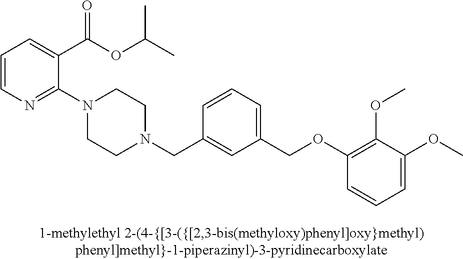 [SMILES: COc1cccc(OCc2cccc(CN3CCN(c4ncccc4C(=O)OC(C)C)CC3)c2)c1OC] | 506.1 | 0.9 |
| Example 213 | 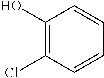 | 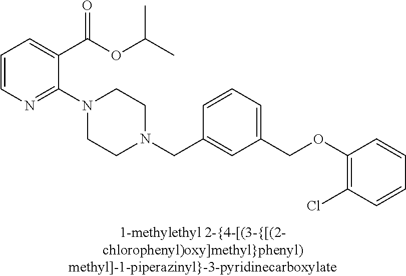 [SMILES: CC(C)OC(=O)c1cccnc1N1CCN(Cc2cccc(COc3ccccc3Cl)c2)CC1] | 480.1 | 0.9 |
| Example 214 | 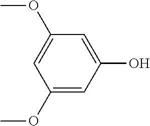 | 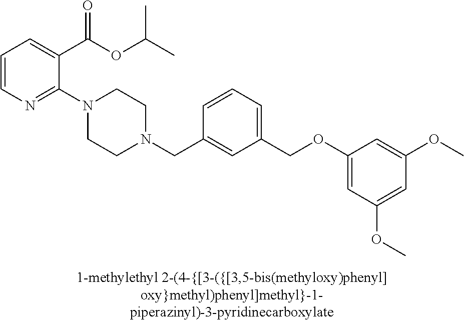 [SMILES: COc1cc(OC)cc(OCc2cccc(CN3CCN(c4ncccc4C(=O)OC(C)C)CC3)c2)c1] | 506.1 | 1.0 |
| Example 215 | 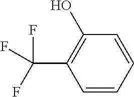 [SMILES: Oc1ccccc1C(F)(F)F] | 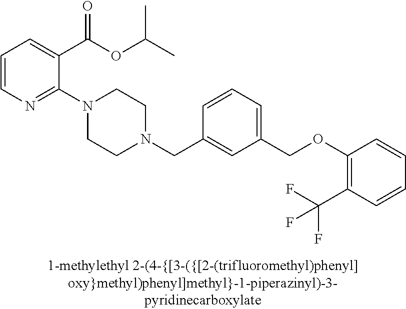 [SMILES: CC(C)OC(=O)c1cccnc1N1CCN(Cc2cccc(COc3ccccc3C(F)(F)F)c2)CC1] | 514.1 | 1.1 |
| Example 216 | 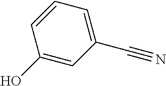 | 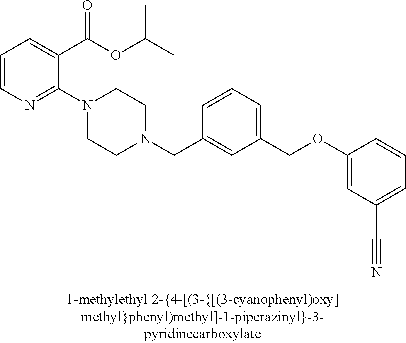 [SMILES: CC(C)OC(=O)c1cccnc1N1CCN(Cc2cccc(COc3cccc(C#N)c3)c2)CC1] | 471.1 | 0.9 |
| Example 217 | 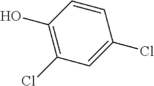 | 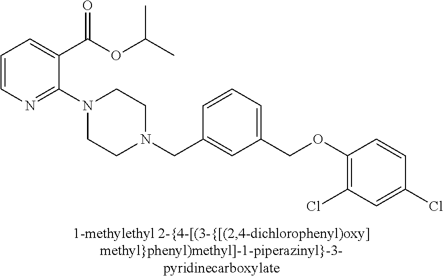 [SMILES: CC(C)OC(=O)c1cccnc1N1CCN(Cc2cccc(COc3ccc(Cl)cc3Cl)c2)CC1] | 514.0 | 1.1 |
| Example 218 | 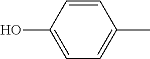 | 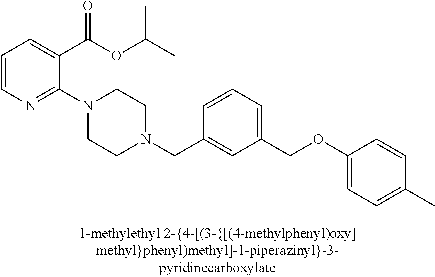 [SMILES: Cc1ccc(OCc2cccc(CN3CCN(c4ncccc4C(=O)OC(C)C)CC3)c2)cc1] | 460.1 | 1.0 |
| Example 219 | 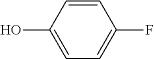 | 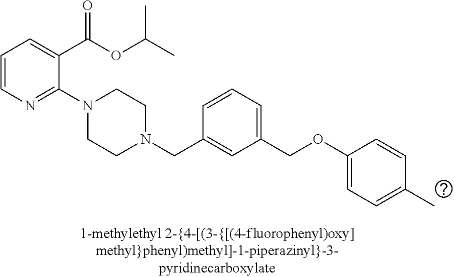 [SMILES: Cc1ccc(OCc2cccc(CN3CCN(c4ncccc4C(=O)OC(C)C)CC3)c2)cc1] | 464.1 | 1.0 |
| Example 220 | 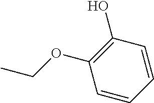 | 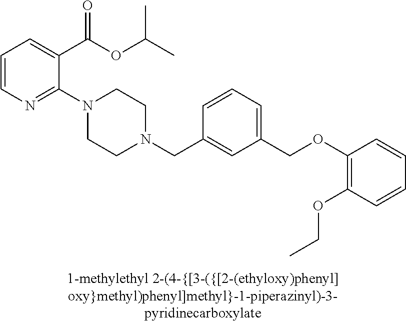 [SMILES: CCOc1ccccc1OCc1cccc(CN2CCN(c3ncccc3C(=O)OC(C)C)CC2)c1] | 490.1 | 1.0 |
| Example 221 | 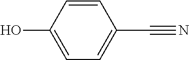 | 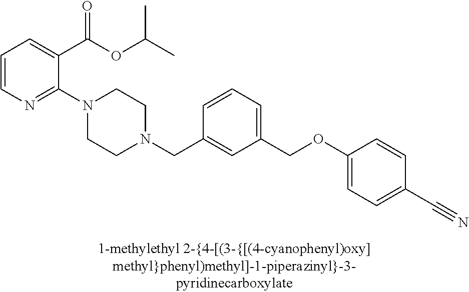 [SMILES: CC(C)OC(=O)c1cccnc1N1CCN(Cc2cccc(COc3ccc(C#N)cc3)c2)CC1] | 471.1 | 0.9 |

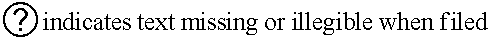

### Table 12

### Example 222

### 1-methylethyl 2-{4-[(4-{[ethyl(3-furanylmethyl)amino]methyl}phenyl)methyl]-1-piperazinyl}-3-pyridinecarboxylate

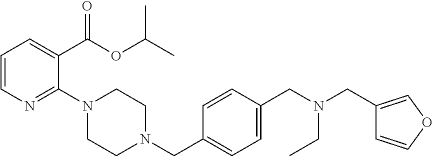 [SMILES: CCN(Cc1ccc(CN2CCN(c3ncccc3C(=O)OC(C)C)CC2)cc1)Cc1ccoc1]

Dissolved 1-methylethyl 2-[4-({4-[(ethylamino)methyl]phenyl}methyl)-1-piperazinyl]-3-pyridinecarboxylate (20.0 mg, 0.05 mmol) and furan-3-carbaldehyde (0.126 mmol) in methanol (3.5 mL) with acetic acid (3.1 mg, 0.050 mmol). The solution was stirred for 1 h at room temperature. Then added sodium cyanoborohydride (11.1 mg, 0.177 mmol) and stirred at room temperature for 12 hr. The polymer was filtered and concentrated the filtrate to give crude product, which was dissolved in DMSO, and purified on a Gilson HPLC (XBridge 19×100 mm 5u preparatory column), eluting at 18 mL/min with a linear gradient running from 40% to 90% acetonitrile and 0.1% aqueous NH₄OH over 15 min. The desired fractions were concentrated under a stream of nitrogen at 45° C., giving the desired product (12.73 mg, 53%). LC/MS: m/z=477.1 [M+H]⁺, Ret. Time: 0.64 min.

Following the procedure as described above in the preparation of 1-methylethyl 2-{4-[(4-{[ethyl(3-furanylmethyl)amino]methyl}phenyl)methyl]-1-piperazinyl}-3-pyridinecarboxylate. 1-methylethyl 2-[4-({4-[(ethylamino)methyl]phenyl}methyl)-1-piperazinyl]-3-pyridine carboxylate (20.0 mg, 0.05 mmol) was reacted with the appropriate aldehydes (0.126 mmol) to yield the examples listed in Table XII.

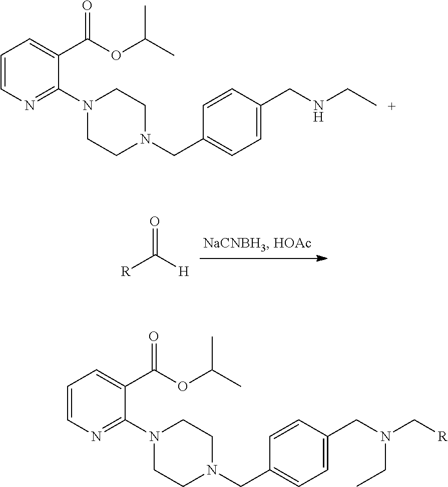

TABLE XII

| | | | LC-MS | |
|---|---|---|---|---|
| | | | m/z | RT |
| Example | Aldehyde | Product | (M + H)⁺ | (min) |
| Example 223 | 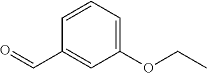 | 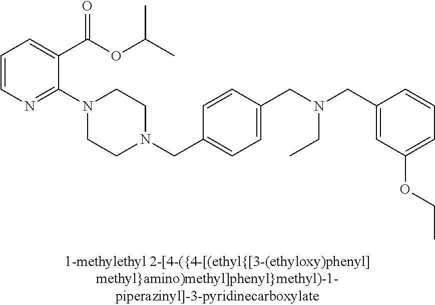 [SMILES: CCOc1cccc(CN(CC)Cc2ccc(CN3CCN(c4ncccc4C(=O)OC(C)C)CC3)cc2)c1] | 531.1 | 0.8 |
| Example 224 | 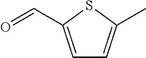 | 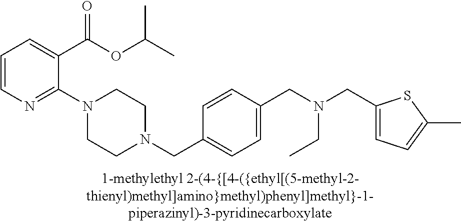 [SMILES: CCN(Cc1ccc(CN2CCN(c3ncccc3C(=O)OC(C)C)CC2)cc1)Cc1ccc(C)s1] | 507.1 | 0.7 |
| Example 225 | 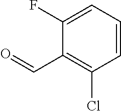 | 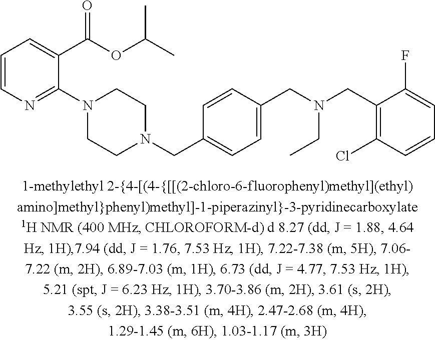 [SMILES: CCN(Cc1ccc(CN2CCN(c3ncccc3C(=O)OC(C)C)CC2)cc1)Cc1c(F)cccc1Cl] | 539.1 | 0.7 |
| Example 226 | 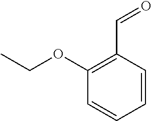 | 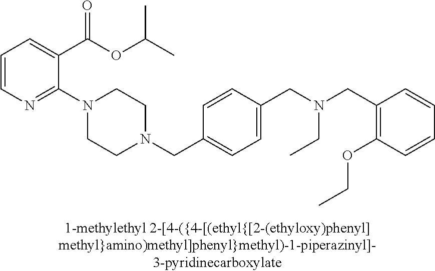 [SMILES: CCOc1ccccc1CN(CC)Cc1ccc(CN2CCN(c3ncccc3C(=O)OC(C)C)CC2)cc1] | 531.1 | 0.8 |
| Example 227 | 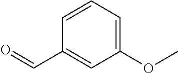 | 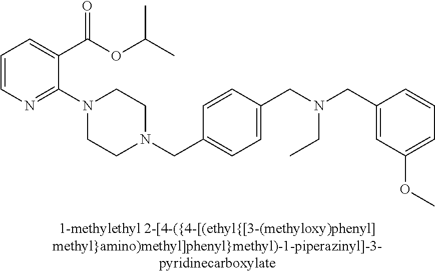 [SMILES: CCN(Cc1ccc(CN2CCN(c3ncccc3C(=O)OC(C)C)CC2)cc1)Cc1cccc(OC)c1] | 517.1 | 0.8 |
| Example 228 | 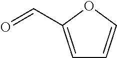 | 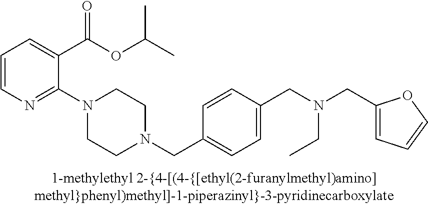 [SMILES: CCN(Cc1ccc(CN2CCN(c3ncccc3C(=O)OC(C)C)CC2)cc1)Cc1ccco1] | 477.1 | 0.6 |
| Example 229 | 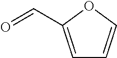 | 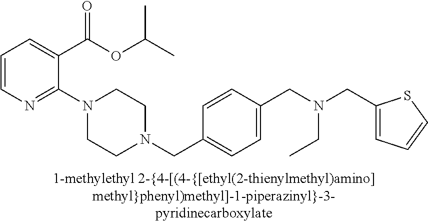 [SMILES: CCN(Cc1ccc(CN2CCN(c3ncccc3C(=O)OC(C)C)CC2)cc1)Cc1cccs1] | 493.1 | 0.7 |

### Table 13

### Example 230

### 1-methylethyl 4-methyl-2-[4-({4-[(methyloxy)carbonyl]phenyl}methyl)-1-piperazinyl]-3-pyridinecarboxylate

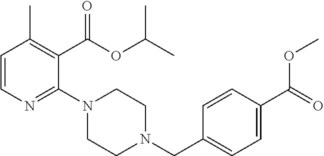 [SMILES: COC(=O)c1ccc(CN2CCN(c3nccc(C)c3C(=O)OC(C)C)CC2)cc1]

Dissolved 1-methylethyl 4-methyl-2-(1-piperazinyl)-3-pyridinecarboxylate (25.0 mg, 0.095 mmol) and methyl 4-formylbenzoate (0.252 mmol) in methanol (2.5 mL) with acetic acid (5.7 mg, 0.095 mmol). The solution was stirred for 4 h at room temperature. Then added sodium cyanoborohydride (20.88 mg, 0.335 mmol) and stirred at room temperature for 12 hr. The polymer was filtered and got the crude products, which was dissolved in DMSO, and purified on a Gilson HPLC (XBridge 19×100 mm 5u preparatory column), eluting at 18 mL/min with a linear gradient running from 25% to 80% acetonitrile and 0.1% aqueous NH₄OH over 15 min. The desired fractions were concentrated under a stream of nitrogen at 45° C., giving the desired product (16.46 mg, 42.1%). LC/MS: m/z=412.2[M+H]⁺, Ret. Time: 0.77 min.

Following the procedure as described above in the preparation of 1-methylethyl 4-methyl-2-[4-({4-[(methyloxy)carbonyl]phenyl}methyl)-1-piperazinyl]-3-pyridine carboxylate, 1-methylethyl 4-methyl-2-(1-piperazinyl)-3-pyridinecarboxylate (25.0 mg, 0.095 mmol) was reacted with the appropriate aldehydes (0.252 mmol) to yield the examples listed in Table XIII.

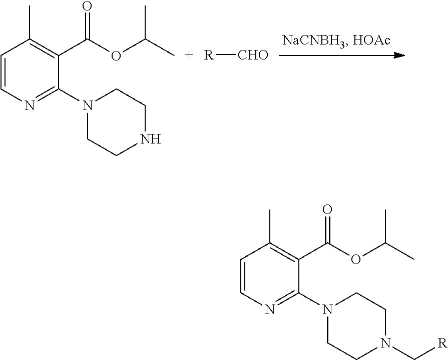

TABLE XIII

| | | | LC-MS | |
|---|---|---|---|---|
| | Aldehyde or | | m/z | RT |
| Example | Ketone | Product | (M + H)⁺ | (min) |
| Example 231 | 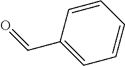 | 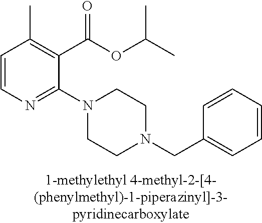 [SMILES: Cc1ccnc(N2CCN(Cc3ccccc3)CC2)c1C(=O)OC(C)C] | 354.18 | 0.8 |
| Example 232 | 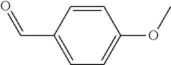 | 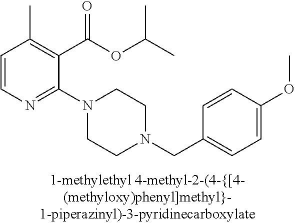 [SMILES: COc1ccc(CN2CCN(c3nccc(C)c3C(=O)OC(C)C)CC2)cc1] | 384.17 | 0.9 |
| Example 233 | 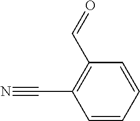 | 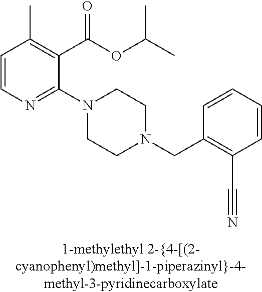 [SMILES: Cc1ccnc(N2CCN(Cc3ccccc3C#N)CC2)c1C(=O)OC(C)C] | 379.16 | 0.7 |
| Example 234 | 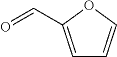 | 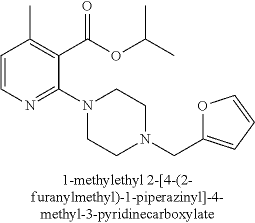 [SMILES: Cc1ccnc(N2CCN(Cc3ccco3)CC2)c1C(=O)OC(C)C] | 344.17 | 0.7 |
| Example 235 | 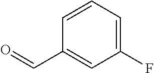 | 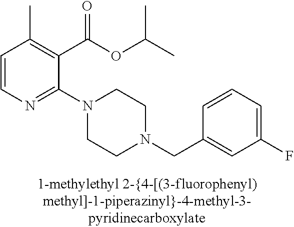 [SMILES: Cc1ccnc(N2CCN(Cc3cccc(F)c3)CC2)c1C(=O)OC(C)C] | 372.14 | 0.8 |
| Example 236 | 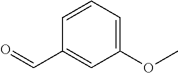 | 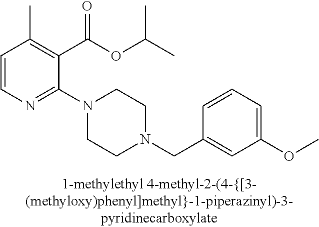 [SMILES: COc1cccc(CN2CCN(c3nccc(C)c3C(=O)OC(C)C)CC2)c1] | 384.2 | 0.8 |
| Example 237 | 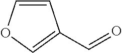 | 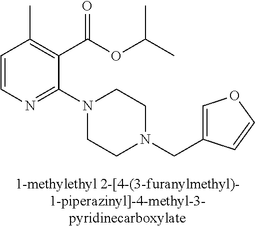 [SMILES: Cc1ccnc(N2CCN(Cc3ccoc3)CC2)c1C(=O)OC(C)C] | 344.16 | 0.7 |
| Example 238 | 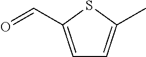 | 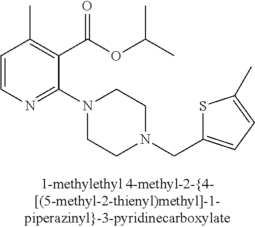 [SMILES: Cc1ccc(CN2CCN(c3nccc(C)c3C(=O)OC(C)C)CC2)s1] | 374.14 | 0.8 |
| Example 239 | 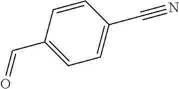 | 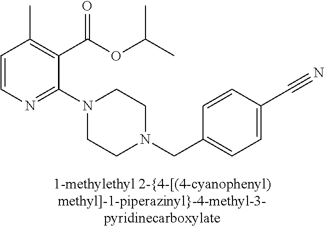 [SMILES: Cc1ccnc(N2CCN(Cc3ccc(C#N)cc3)CC2)c1C(=O)OC(C)C] | 379.17 | 0.7 |
| Example 240 | 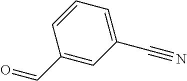 | 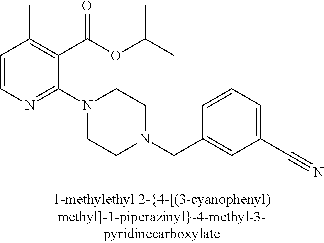 [SMILES: Cc1ccnc(N2CCN(Cc3cccc(C#N)c3)CC2)c1C(=O)OC(C)C] | 379.17 | 0.7 |
| Example 241 | 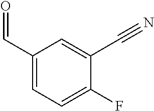 [SMILES: N#Cc1cc(C=O)ccc1F] | 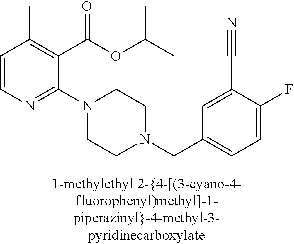 [SMILES: Cc1ccnc(N2CCN(Cc3ccc(F)c(C#N)c3)CC2)c1C(=O)OC(C)C] | 397.14 | 0.8 |
| Example 242 | 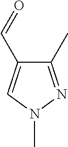 | 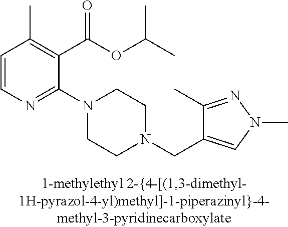 [SMILES: Cc1ccnc(N2CCN(Cc3cn(C)nc3C)CC2)c1C(=O)OC(C)C] | 372.21 | 0.8 |
| Example 243 | 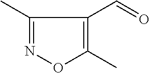 | 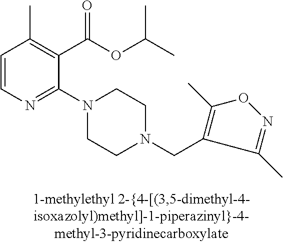 [SMILES: Cc1ccnc(N2CCN(Cc3c(C)noc3C)CC2)c1C(=O)OC(C)C] | 373.16 | 0.7 |
| Example 244 | 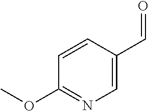 | 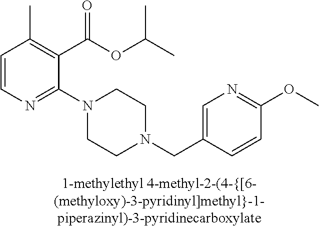 [SMILES: COc1ccc(CN2CCN(c3nccc(C)c3C(=O)OC(C)C)CC2)cn1] | 385.17 | 0.7 |
| Example 245 | 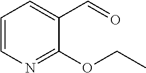 | 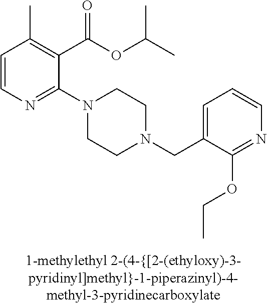 [SMILES: CCOc1ncccc1CN1CCN(c2nccc(C)c2C(=O)OC(C)C)CC1] | 399.21 | 0.8 |
| Example 246 | 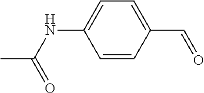 | 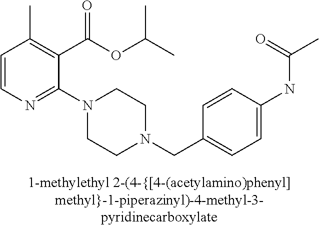 [SMILES: CC(=O)Nc1ccc(CN2CCN(c3nccc(C)c3C(=O)OC(C)C)CC2)cc1] | 411.18 | 0.7 |
| Example 247 | 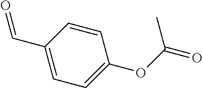 | 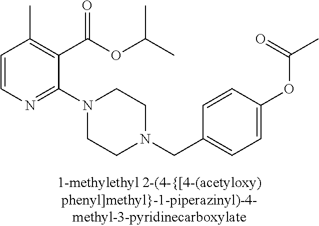 [SMILES: CC(=O)Oc1ccc(CN2CCN(c3nccc(C)c3C(=O)OC(C)C)CC2)cc1] | 412.2 | 0.8 |
| Example 248 | 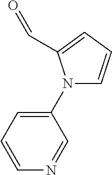 [SMILES: O=Cc1cccn1-c1cccnc1] | 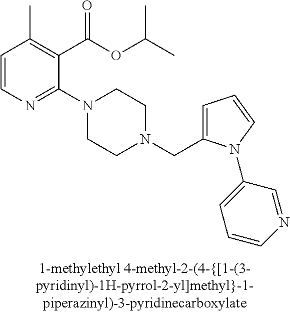 [SMILES: Cc1ccnc(N2CCN(Cc3cccn3-c3cccnc3)CC2)c1C(=O)OC(C)C] | 420.21 | 0.7 |
| Example 249 | 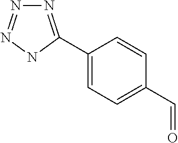 [SMILES: O=Cc1ccc(-c2nnn[nH]2)cc1] | 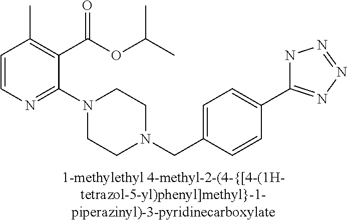 [SMILES: Cc1ccnc(N2CCN(Cc3ccc(-c4nnn[nH]4)cc3)CC2)c1C(=O)OC(C)C] | 582.8 | 0.7 |
| Example 250 | 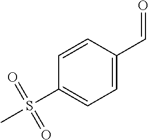 | 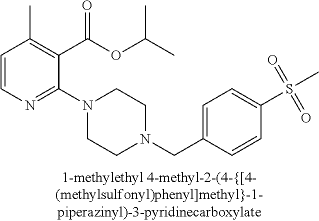 [SMILES: Cc1ccnc(N2CCN(Cc3ccc(S(C)(=O)=O)cc3)CC2)c1C(=O)OC(C)C] | 432.15 | 0.8 |
| Example 251 | 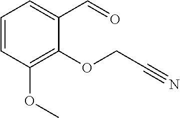 | 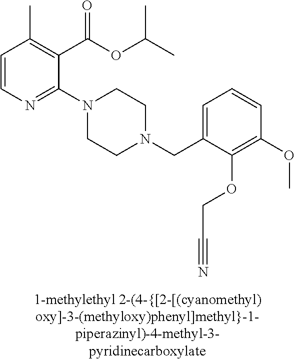 [SMILES: COc1cccc(CN2CCN(c3nccc(C)c3C(=O)OC(C)C)CC2)c1OCC#N] | 439.19 | 0.8 |
| Example 252 | 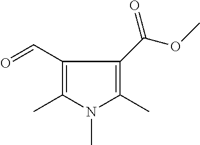 | 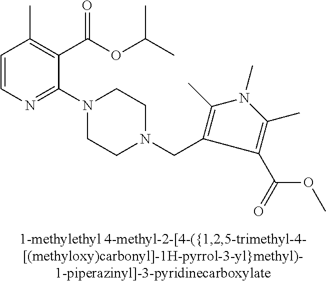 [SMILES: COC(=O)c1c(CN2CCN(c3nccc(C)c3C(=O)OC(C)C)CC2)c(C)n(C)c1C] | 443.21 | 0.8 |
| Example 253 | 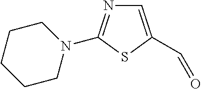 [SMILES: O=Cc1cnc(N2CCCCC2)s1] | 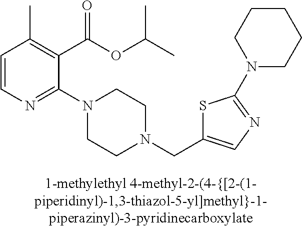 [SMILES: Cc1ccnc(N2CCN(Cc3cnc(N4CCCCC4)s3)CC2)c1C(=O)OC(C)C] | 444.2 | 0.8 |
| Example 254 | 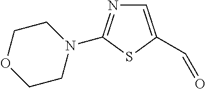 [SMILES: O=Cc1cnc(N2CCOCC2)s1] | 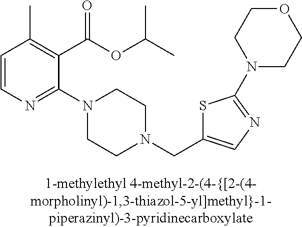 [SMILES: Cc1ccnc(N2CCN(Cc3cnc(N4CCOCC4)s3)CC2)c1C(=O)OC(C)C] | 446.17 | 0.7 |
| Example 255 | 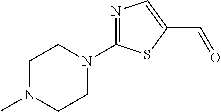 | 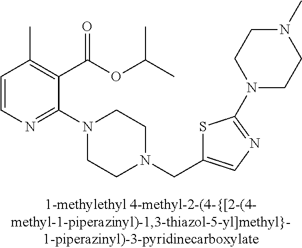 [SMILES: Cc1ccnc(N2CCN(Cc3cnc(N4CCN(C)CC4)s3)CC2)c1C(=O)OC(C)C] | 459.2 | 0.6 |
| Example 256 | 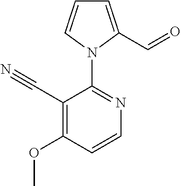 [SMILES: COc1ccnc(-n2cccc2C=O)c1C#N] | 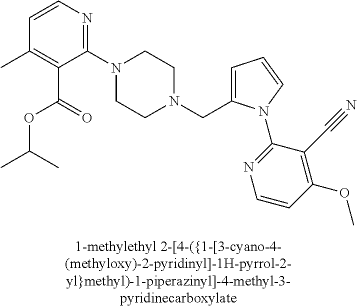 | 475.17 | 0.8 |

### Table 14

### Example 257

### 1-methylethyl 2-{methyl[(3R)-1-({2-[(trifluoromethyl)oxy]phenyl}methyl)-3-pyrrolidinyl]amino}-3-pyridinecarboxylate

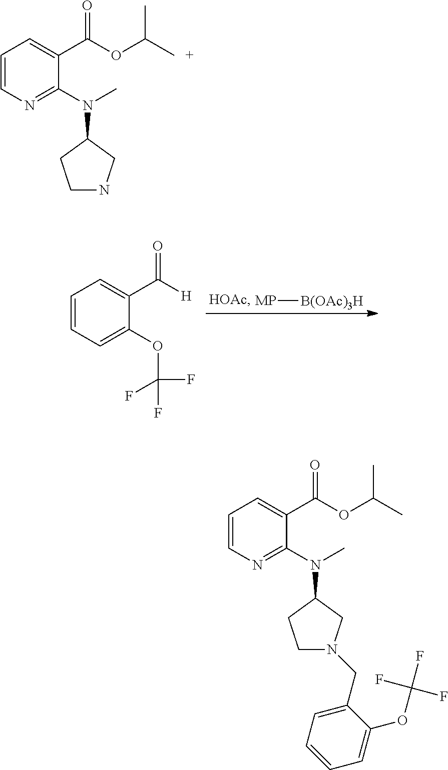

In an A-vial, 2-[(trifluoromethyl)oxy]benzaldehyde (45.1 mg, 0.237 mmol) and 1-methylethyl 2-{methyl[(3R)-3-pyrrolidinyl]amino}-3-pyridinecarboxylate (25 mg, 0.095 mmol) were added to the solution of with acetic acid (5.7 mg, 0.095 mmol) in dimethyl sulfoxide (DMSO) (1.5 ml). The solution was stirred for 1 h at room temperature. Then MP-B(OAc)₃H (111 mg, 0.475 mmol) was added. The resulted solution was stirred at room temperature for 12 hours. The polymer was filtered and the crude product was dissolved in DMSO, and purified on a Gilson HPLC (XBridge 19×100 mm 5u preparatory column), eluting with acetonitrile, water 0.1% NH4OH. The desired fractions were concentrated under a stream of nitrogen at 50° C., giving 4.09 mg (10.9%) of the titled compound. LC-MS m/z 438.17 (M+H)⁺, 1.0 min (ret time).

Following the procedure as described above in the preparation of 1-methylethyl 2-{methyl[(3R)-1-({2-[(trifluoromethyl)oxy]phenyl}methyl)-3-pyrrolidinyl]amino}-3-pyridinecarboxylate,

1-methylethyl 2-{methyl[(3R)-3-pyrrolidinyl]amino}-3-pyridinecarboxylate (25 mg, 0.095 mmol) was reacted with the appropriate aldehyde to yield the examples listed in Table XIV.

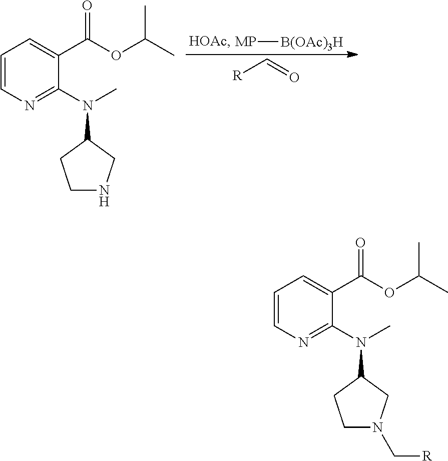

TABLE XIV

| | | | LC-MS | |
|---|---|---|---|---|
| | | | m/z | RT |
| Example | Aldehyde | Product Name | (M + H)⁺ | (min) |
| Example 258 | 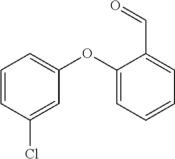 [SMILES: O=Cc1ccccc1Oc1cccc(Cl)c1] | 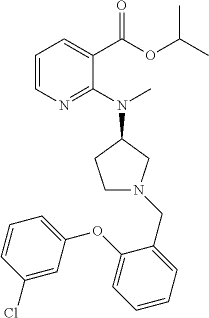 [SMILES: CC(C)OC(=O)c1cccnc1N(C)[C@@H]1CCN(Cc2ccccc2Oc2cccc(Cl)c2)C1] 1-methylethyl 2-[[(3R)-1-({2-[(3- chlorophenyl)oxy]phenyl}methyl)-3- pyrrolidinyl](methyl)amino]-3- pyridinecarboxylate | 480.19 | 1.2 |
| Example 259 | 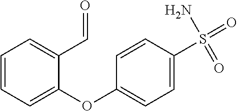 [SMILES: NS(=O)(=O)c1ccc(Oc2ccccc2C=O)cc1] | 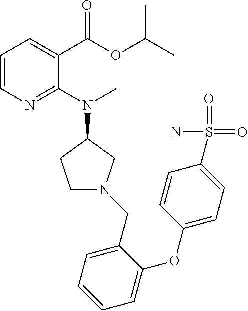 [SMILES: CC(C)OC(=O)c1cccnc1N(C)[C@@H]1CCN(Cc2ccccc2Oc2ccc(S(N)(=O)=O)cc2)C1] 1-methylethyl 2-[{(3R)-1-[(2-{[4- (aminosulfonyl)phenyl]oxy}phenyl) methyl]-3-pyrrolidinyl}(methyl)amino]- 3-pyridinecarboxylate | 525.2 | 0.9 |
| Example 260 | 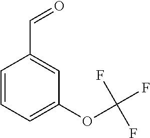 [SMILES: O=Cc1cccc(OC(F)(F)F)c1] | 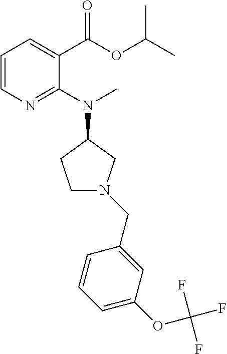 [SMILES: CC(C)OC(=O)c1cccnc1N(C)[C@@H]1CCN(Cc2cccc(OC(F)(F)F)c2)C1] 1-methylethyl 2-{methyl[(3R)-1-({3- [(trifluoromethyl)oxy]phenyl}methyl)- 3-pyrrolidinyl]amino}-3- pyridinecarboxylate | 438.17 | 1.0 |
| Example 261 | 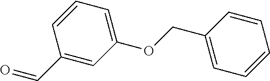 [SMILES: O=Cc1cccc(OCc2ccccc2)c1] | 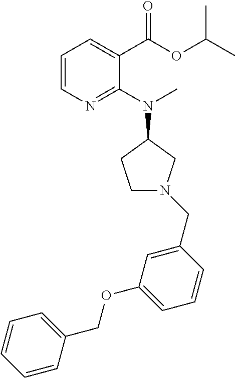 [SMILES: CC(C)OC(=O)c1cccnc1N(C)[C@@H]1CCN(Cc2cccc(OCc3ccccc3)c2)C1] 1-methylethyl 2-{methyl[(3R)-1-({3- [(phenylmethyl)oxy]phenyl}methyl)- 3-pyrrolidinyl]amino}-3- pyridinecarboxylate | 460.24 | 1.1 |
| Example 262 | 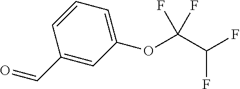 [SMILES: O=Cc1cccc(OC(F)(F)C(F)F)c1] | 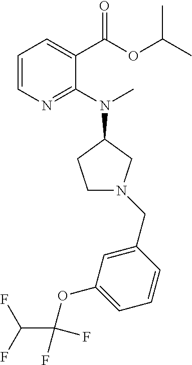 [SMILES: CC(C)OC(=O)c1cccnc1N(C)[C@@H]1CCN(Cc2cccc(OC(F)(F)C(F)F)c2)C1] 1-methylethyl 2-{methyl[(3R)- 1-({3-[(1,1,2,2- tetrafluoroethyl)oxy]phenyl}methyl)- 3-pyrrolidinyl]amino}-3- pyridinecarboxylate | 470.19 | 1.0 |
| Example 263 | 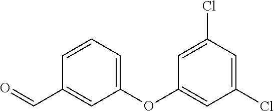 [SMILES: O=Cc1cccc(Oc2cc(Cl)cc(Cl)c2)c1] | 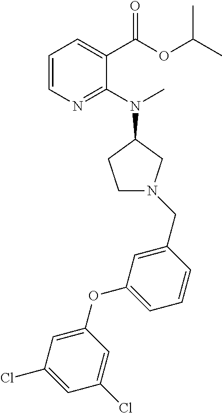 [SMILES: CC(C)OC(=O)c1cccnc1N(C)[C@@H]1CCN(Cc2cccc(Oc3cc(Cl)cc(Cl)c3)c2)C1] 1-methylethyl 2-[[(3R)-1-({3-[(3,5- dichlorophenyl)oxy]phenyl}methyl)-3- pyrrolidinyl](methyl)amino]-3- pyridinecarboxylate | 514.15 | 1.3 |
| Example 264 | 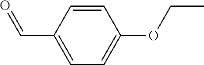 | 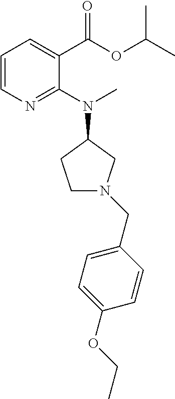 [SMILES: CCOc1ccc(CN2CC[C@@H](N(C)c3ncccc3C(=O)OC(C)C)C2)cc1] 1-methylethyl 2-[((3R)-1-{[4- (ethyloxy)phenyl]methyl}-3- pyrrolidinyl)(methyl)amino]-3- pyridinecarboxylate | 398.22 | 1.0 |
| Example 265 | 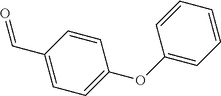 [SMILES: O=Cc1ccc(Oc2ccccc2)cc1] | 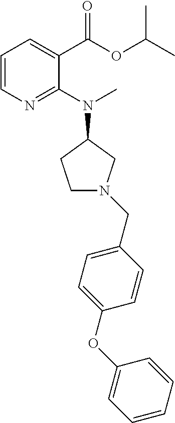 [SMILES: CC(C)OC(=O)c1cccnc1N(C)[C@@H]1CCN(Cc2ccc(Oc3ccccc3)cc2)C1] 1-methylethyl 2-[methyl((3R)-1-{[4- (phenyloxy)phenyl]methyl}-3- pyrrolidinyl)amino]-3- pyridinecarboxylate | 446.23 | 1.1 |
| Example 266 | 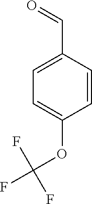 [SMILES: O=Cc1ccc(OC(F)(F)F)cc1] | 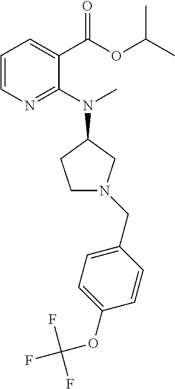 [SMILES: CC(C)OC(=O)c1cccnc1N(C)[C@@H]1CCN(Cc2ccc(OC(F)(F)F)cc2)C1] 1-methylethyl 2-{methyl[(3R)-1-({4- [(trifluoromethyl)oxy]phenyl}methyl)- 3-pyrrolidinyl]amino}-3- pyridinecarboxylate | 438.17 | 1.0 |
| Example 267 | 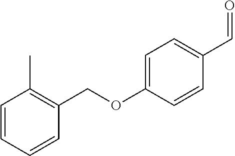 [SMILES: Cc1ccccc1COc1ccc(C=O)cc1] | 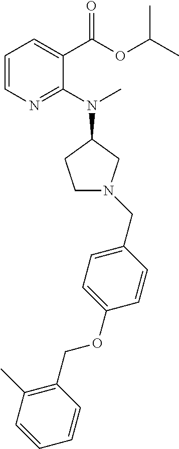 [SMILES: Cc1ccccc1COc1ccc(CN2CC[C@@H](N(C)c3ncccc3C(=O)OC(C)C)C2)cc1] 1-methylethyl 2-(methyl-{(3R)-1-[(4-{[(2- methylphenyl)methyl]oxy}phenyl) methyl]-3-pyrrolidinyl}amino)-3- pyridinecarboxylate | 474.26 | 1.1 |
| Example 268 | 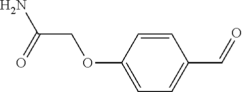 [SMILES: NC(=O)COc1ccc(C=O)cc1] | 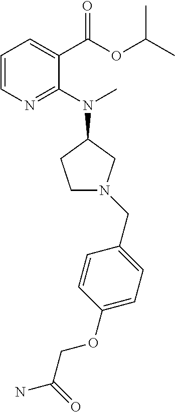 [SMILES: CC(C)OC(=O)c1cccnc1N(C)[C@@H]1CCN(Cc2ccc(OCC(N)=O)cc2)C1] 1-methylethyl 2-[[(3R)-1-({4-[(2-amino- 2-oxoethyl)oxy]phenyl}methyl)- 3-pyrrolidinyl](methyl)amino]- 3-pyridinecarboxylate | 427.24 | 0.7 |
| Example 269 | 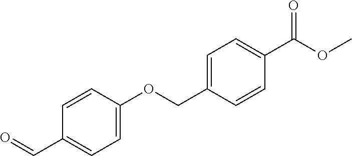 [SMILES: COC(=O)c1ccc(COc2ccc(C=O)cc2)cc1] | 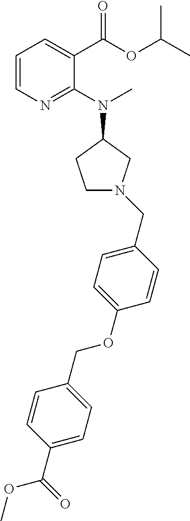 [SMILES: COC(=O)c1ccc(COc2ccc(CN3CC[C@@H](N(C)c4ncccc4C(=O)OC(C)C)C3)cc2)cc1] 1-methylethyl 2-{methyl[(3R)-1-({4-[({4- [(methyloxy)carbonyl]phenyl} methyl)oxy]phenyl}methyl)-3- pyrrolidinyl]amino}-3-pyridinecarboxylate | 518.24 | 1.1 |
| Example 270 | 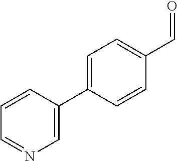 [SMILES: O=Cc1ccc(-c2cccnc2)cc1] | 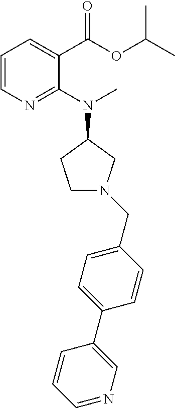 [SMILES: CC(C)OC(=O)c1cccnc1N(C)[C@@H]1CCN(Cc2ccc(-c3cccnc3)cc2)C1] 1-methylethyl 2-[methyl((3R)-1-{[4-(3- pyridinyl)phenyl]methyl}-3- pyrrolidinyl)amino]-3- pyridinecarboxylate | 431.24 | 0.7 |
| Example 271 | 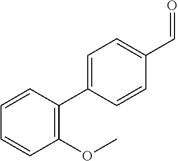 [SMILES: COc1ccccc1-c1ccc(C=O)cc1] | 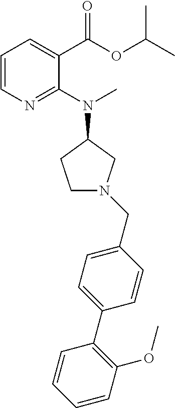 [SMILES: COc1ccccc1-c1ccc(CN2CC[C@@H](N(C)c3ncccc3C(=O)OC(C)C)C2)cc1] 1-methylethyl 2-[methyl((3R)-1-{[2′- (methyloxy)-4-biphenylyl]methyl}-3- pyrrolidinyl)amino]-3- pyridinecarboxylate | 460.22 | 1.0 |
| Example 272 | 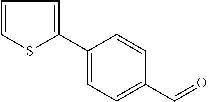 [SMILES: O=Cc1ccc(-c2cccs2)cc1] | 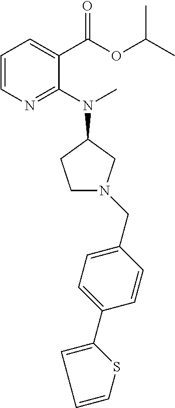 [SMILES: CC(C)OC(=O)c1cccnc1N(C)[C@@H]1CCN(Cc2ccc(-c3cccs3)cc2)C1] 1-methylethyl 2-[methyl((3R)-1-{[4-(2- thienyl)phenyl]methyl}-3- pyrrolidinyl)amino]-3- pyridinecarboxylate | 436.18 | 1.0 |
| Example 273 | 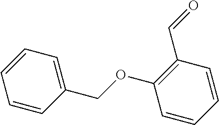 [SMILES: O=Cc1ccccc1OCc1ccccc1] | 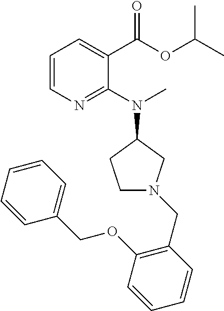 [SMILES: CC(C)OC(=O)c1cccnc1N(C)[C@@H]1CCN(Cc2ccccc2OCc2ccccc2)C1] 1-methylethyl 2-{methyl[(3R)-1-({2- [(phenylmethyl)oxy]phenyl}methyl)- 3-pyrrolidinyl]amino}-3- pyridinecarboxylate | 460.23 | 1.1 |
| Example 274 | 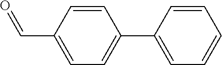 [SMILES: O=Cc1ccc(-c2ccccc2)cc1] | 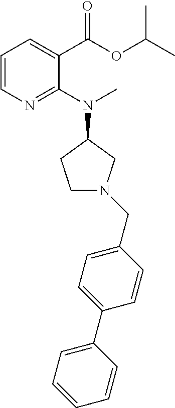 [SMILES: CC(C)OC(=O)c1cccnc1N(C)[C@@H]1CCN(Cc2ccc(-c3ccccc3)cc2)C1] 1-methylethyl 2-[[(3R)-1-(4- biphenylylmethyl)-3- pyrrolidinyl](methyl)amino]-3- pyridinecarboxylate | 430.23 | 1.1 |
| Example 275 | 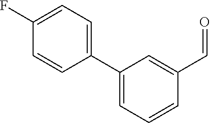 [SMILES: O=Cc1cccc(-c2ccc(F)cc2)c1] | 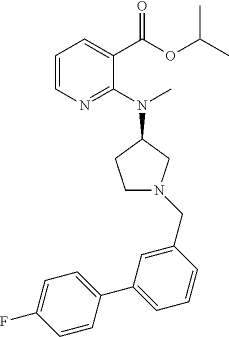 [SMILES: CC(C)OC(=O)c1cccnc1N(C)[C@@H]1CCN(Cc2cccc(-c3ccc(F)cc3)c2)C1] 1-methylethyl 2-[{(3R)-1-[(4′- fluoro-3-biphenylyl)methyl]-3- pyrrolidinyl}(methyl)amino]-3- pyridinecarboxylate | 448.24 | 1.1 |
| Example 276 | 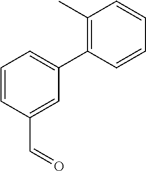 | 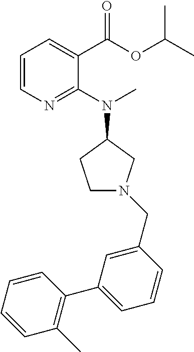 [SMILES: Cc1ccccc1-c1cccc(CN2CC[C@@H](N(C)c3ncccc3C(=O)OC(C)C)C2)c1] 1-methylethyl 2-(methyl{(3R)- 1-[(2′-methyl-3-biphenylyl)methyl]-3- pyrrolidinyl}amino)-3- pyridinecarboxylate | 444.24 | 1.1 |
| Example 277 | 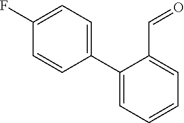 [SMILES: O=Cc1ccccc1-c1ccc(F)cc1] | 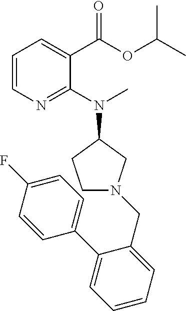 [SMILES: CC(C)OC(=O)c1cccnc1N(C)[C@@H]1CCN(Cc2ccccc2-c2ccc(F)cc2)C1] 1-methylethyl 2-[{(3R)-1-[(4′- fluoro-2-biphenylyl)methyl]-3- pyrrolidinyl}(methyl)amino]-3- pyridinecarboxylate | 448.24 | 1.0 |
| Example 278 | 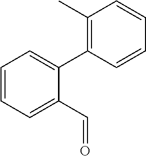 | 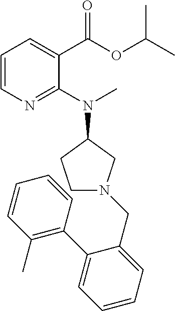 [SMILES: Cc1ccccc1-c1ccccc1CN1CC[C@@H](N(C)c2ncccc2C(=O)OC(C)C)C1] 1-methylethyl 2-(methyl{(3R)- 1-[(2′-methyl-2-biphenylyl)methyl]-3- pyrrolidinyl}amino)-3- pyridinecarboxylate | 444.25 | 1.1 |
| Example 279 | 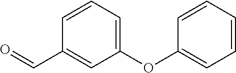 [SMILES: O=Cc1cccc(Oc2ccccc2)c1] | 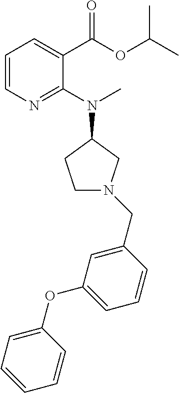 [SMILES: CC(C)OC(=O)c1cccnc1N(C)[C@@H]1CCN(Cc2cccc(Oc3ccccc3)c2)C1] 1-methylethyl 2-[methyl((3R)-1-{[3- (phenyloxy)phenyl]methyl}-3- pyrrolidinyl)amino]-3- pyridinecarboxylate | 446.21 | 1.1 |
| Example 280 | 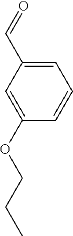 | 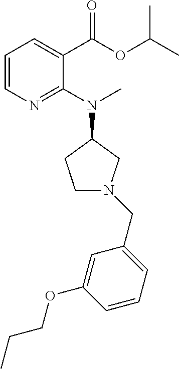 [SMILES: CCCOc1cccc(CN2CC[C@@H](N(C)c3ncccc3C(=O)OC(C)C)C2)c1] 1-methylethyl 2-[methyl((3R)-1-{[3- (propyloxy)phenyl]methyl}-3- pyrrolidinyl)amino]-3- pyridinecarboxylate | 412.22 | 1.0 |
| Example 281 | 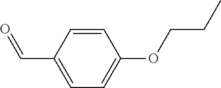 | 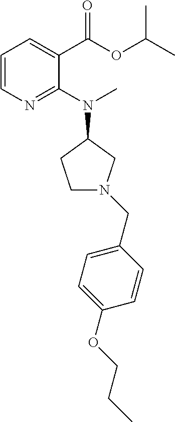 [SMILES: CCCOc1ccc(CN2CC[C@@H](N(C)c3ncccc3C(=O)OC(C)C)C2)cc1] 1-methylethyl 2-[methyl((3R)-1-{[4- (propyloxy)phenyl]methyl}-3- pyrrolidinyl)amino]-3- pyridinecarboxylate | 412.23 | 1.1 |

### Table 15

Following the procedure as described above in the preparation of 1-methylethyl 2-{methyl[(3R)-1-({2-[(trifluoromethyl)oxy]phenyl}methyl)-3-pyrrolidinyl]amino}-3-pyridinecarboxylate, 1-methylethyl 2-[(3R)-3-(ethylamino)-1-pyrrolidinyl]-4-methyl-3-pyridine carboxylate (30 mg, 0.103 mmol) was reacted with the appropriate aldehyde or ketone to yield the examples listed in Table XV.

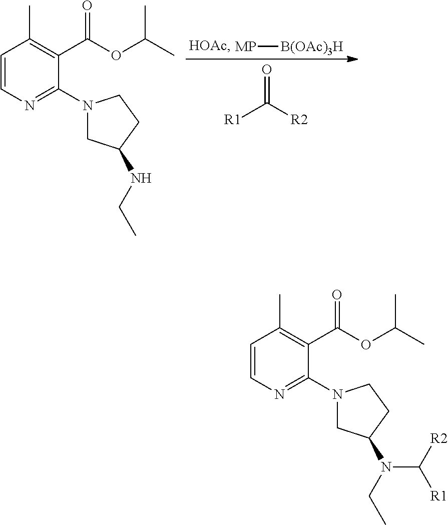

TABLE XV

| | | | LC-MS | |
|---|---|---|---|---|
| | Aldehyde | | m/z | RT |
| Example | (ketone) | Product Name | (M + H)⁺ | (min) |
| Example 282 | 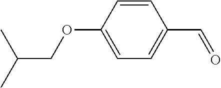 | 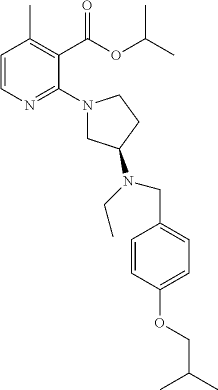 [SMILES: CCN(Cc1ccc(OCC(C)C)cc1)[C@@H]1CCN(c2nccc(C)c2C(=O)OC(C)C)C1] 1-methylethyl2-{(3R)-3-[ethyl({4-[(2- methylpropyl)oxy]phenyl}methyl) amino]-1-pyrrolidinyl}-4- methyl-4-pyridinecarboxylate | 454.2 | 1.0 |
| Example 283 | 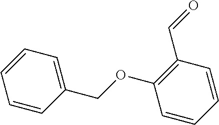 [SMILES: O=Cc1ccccc1OCc1ccccc1] | 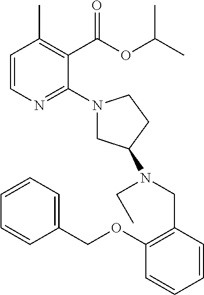 [SMILES: CCN(Cc1ccccc1OCc1ccccc1)[C@@H]1CCN(c2nccc(C)c2C(=O)OC(C)C)C1] 1-methylethyl 2-{(3R)-3-[ethyl({2- [(phenylmethyl)oxy]phenyl}methyl)amino]- 1-pyrrolidinyl}-4-methyl-3-pyridinecarboxylate | 488.17 | 0.9 |
| Example 284 | 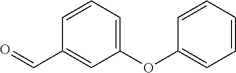 [SMILES: O=Cc1cccc(Oc2ccccc2)c1] | 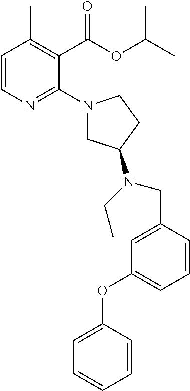 [SMILES: CCN(Cc1cccc(Oc2ccccc2)c1)[C@@H]1CCN(c2nccc(C)c2C(=O)OC(C)C)C1] 1-methylethyl 2-[(3R)-3-(ethyl{[3- (phenyloxy)phenyl]methyl}amino)- 1-pyrrolidinyl]-4-methyl-3-pyridinecarboxylate | 474.15 | 0.9 |
| Example 285 | 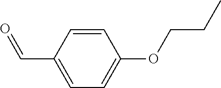 | 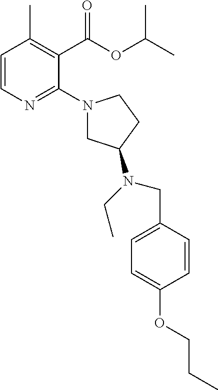 [SMILES: CCCOc1ccc(CN(CC)[C@@H]2CCN(c3nccc(C)c3C(=O)OC(C)C)C2)cc1] 1-methylethyl 2-[(3R)-3-(ethyl{[4- (propyloxy)phenyl]methyl}amino)- 1-pyrrolidinyl]-4-methyl-3-pyridinecarboxylate | 440.17 | 0.9 |
| Example 286 | 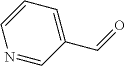 | 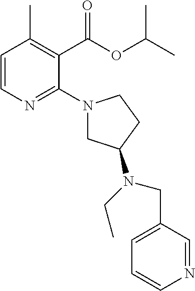 [SMILES: CCN(Cc1cccnc1)[C@@H]1CCN(c2nccc(C)c2C(=O)OC(C)C)C1] 1-methylethyl 2-{(3R)-3-[ethyl(3-pyridinylmethyl)amino]- 1-pyrrolidinyl}-4-methyl-3-pyridinecarboxylate | 383.11 | 0.6 |
| Example 287 | 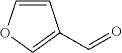 | 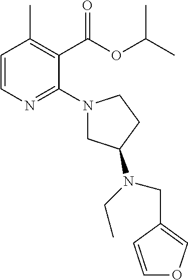 [SMILES: CCN(Cc1ccoc1)[C@@H]1CCN(c2nccc(C)c2C(=O)OC(C)C)C1] 1-methylethyl 2-{(3R)-3-[ethyl(3-furanylmethyl)amino]- 1-pyrrolidinyl}-4-methyl-3-pyridinecarboxylate | 372.09 | 0.7 |
| Example 288 | 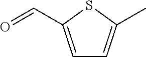 | 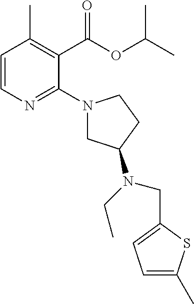 [SMILES: CCN(Cc1ccc(C)s1)[C@@H]1CCN(c2nccc(C)c2C(=O)OC(C)C)C1] 1-methylethyl 2-((3R)-3-{ethyl[(5-methyl-2- thienyl)methyl]amino}-1-pyrrolidinyl)-4-methyl-3- pyridinecarboxylate | 402.1 | 0.8 |
| Example 289 | 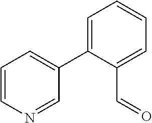 [SMILES: O=Cc1ccccc1-c1cccnc1] | 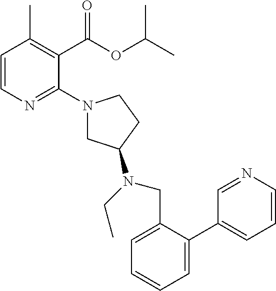 [SMILES: CCN(Cc1ccccc1-c1cccnc1)[C@@H]1CCN(c2nccc(C)c2C(=O)OC(C)C)C1] 1-methylethyl 2-[(3R)-3-(ethyl{[2-(3- pyridinyl)phenyl]methyl}amino)- 1-pyrrolidinyl]-4-methyl-3-pyridinecarboxylate | 459.1 | 0.7 |
| Example 290 | 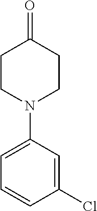 [SMILES: O=C1CCN(c2cccc(Cl)c2)CC1] | 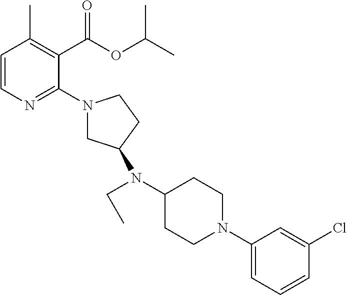 [SMILES: CCN(C1CCN(c2cccc(Cl)c2)CC1)[C@@H]1CCN(c2nccc(C)c2C(=O)OC(C)C)C1] 1-methylethyl 2-{(3R)-3-[[1-(3-chlorophenyl)-4- piperidinyl](ethyl)amino]-1-pyrrolidinyl}-4-methyl-3- pyridinecarboxylate | 485.1 | 1.0 |
| Example 291 | 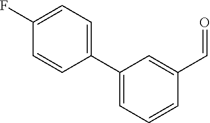 [SMILES: O=Cc1cccc(-c2ccc(F)cc2)c1] | 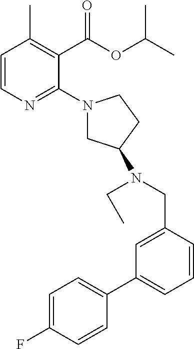 [SMILES: CCN(Cc1cccc(-c2ccc(F)cc2)c1)[C@@H]1CCN(c2nccc(C)c2C(=O)OC(C)C)C1] 1-methyelthyl 2-((3R)-3-{ethyl[(4′-fluoro-3- biphenylyl)methyl]amino}-1-pyrrolidinyl)-4-methyl-3- pyridinecarboxylate | 476.1 | 1.0 |
| Example 292 | 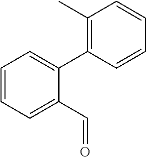 | 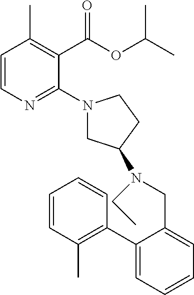 [SMILES: CCN(Cc1ccccc1-c1ccccc1C)[C@@H]1CCN(c2nccc(C)c2C(=O)OC(C)C)C1] 1-methylethyl 2-((3R)-3-{ethyl[(2′-methyl-2- biphenylyl)methyl]amino}-1-pyrrolidinyl)-4-methyl-3- pyridinecarboxylate | 472.14 | 1.0 |
| Example 293 | 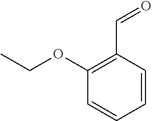 | 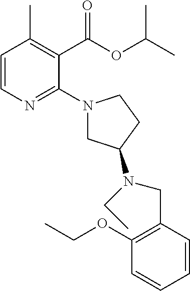 [SMILES: CCOc1ccccc1CN(CC)[C@@H]1CCN(c2nccc(C)c2C(=O)OC(C)C)C1] 1-methylethyl 2-[(3R)-3-(ethyl{[2- (ethyloxy)phenyl]methyl}amino)- 1-pyrrolidinyl]-4-methyl-3-pyridinecarboxylate | 426.15 | 0.9 |
| Example 294 | 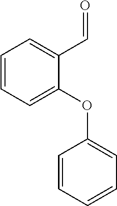 [SMILES: O=Cc1ccccc1Oc1ccccc1] | 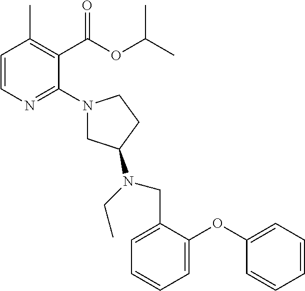 [SMILES: CCN(Cc1ccccc1Oc1ccccc1)[C@@H]1CCN(c2nccc(C)c2C(=O)OC(C)C)C1] 1-methylethyl 2-[(3R)-3-(ethyl{[2- (phenyloxy)phenyl]methyl}amino)- 1-pyrrolidinyl]-4-methyl-3-pyridinecarboxylate | 474.16 | 0.9 |
| Example 295 | 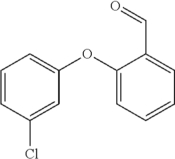 [SMILES: O=Cc1ccccc1Oc1cccc(Cl)c1] | 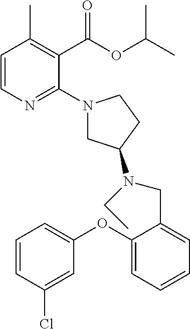 [SMILES: CCN(Cc1ccccc1Oc1cccc(Cl)c1)[C@@H]1CCN(c2nccc(C)c2C(=O)OC(C)C)C1] 1-methyelthyl 2-{(3R)-3-[({2-[(3- chlorophenyl)oxy]phenyl}methyl) (ethyl)amino]-1-pyrrolidinyl}-4- methyl-3-pyridinecarboxylate | 508 | 1.0 |
| Example 296 | 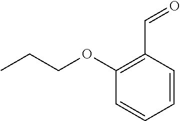 | 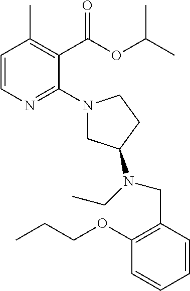 [SMILES: CCCOc1ccccc1CN(CC)[C@@H]1CCN(c2nccc(C)c2C(=O)OC(C)C)C1] 1-methylethyl 2-[(3R)-3-(ethyl{[2- (propyloxy)phenyl]methyl}amino)- 1-pyrrolidinyl]-4-methyl-3-pyridinecarboxylate | 440.17 | 0.9 |
| Example 297 | 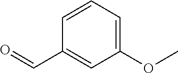 | 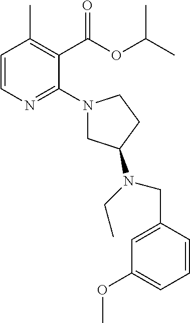 [SMILES: CCN(Cc1cccc(OC)c1)[C@@H]1CCN(c2nccc(C)c2C(=O)OC(C)C)C1] 1-methylethyl 2-[(3R)-3-(ethyl{[3- (methyloxy)phenyl]methyl}amino)- 1-pyrrolidinyl]-4-methyl-3-pyridinecarboxylate | 412.14 | 0.8 |
| Example 298 | 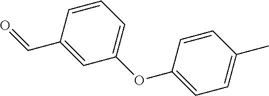 [SMILES: Cc1ccc(Oc2cccc(C=O)c2)cc1] | 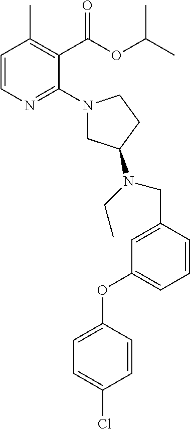 [SMILES: CCN(Cc1cccc(Oc2ccc(Cl)cc2)c1)[C@@H]1CCN(c2nccc(C)c2C(=O)OC(C)C)C1] 1-methylethyl 2-{(3R)-3-[({3-[(4- chlorophenyl)oxy]phenyl}methyl) (ethyl)amino]-1-pyrrolidinyl}-4- methyl-3-pyridinecarboxylate | 508 | 1.1 |
| Example 299 | 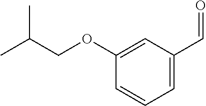 | 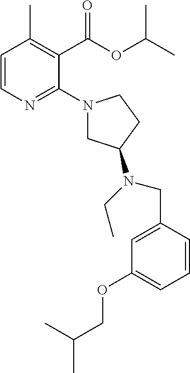 [SMILES: CCN(Cc1cccc(OCC(C)C)c1)[C@@H]1CCN(c2nccc(C)c2C(=O)OC(C)C)C1] 1-methylethyl 2-{(3R)-3-[ethyl({3-[(2- methylpropyl)oxy]phenyl}methyl) amino]-1-pyrrolidinyl}-4- methyl-3-pyridinecarboxylate | 454.19 | 1.0 |
| Example 300 | 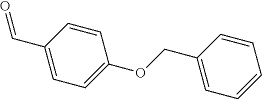 [SMILES: O=Cc1ccc(OCc2ccccc2)cc1] | 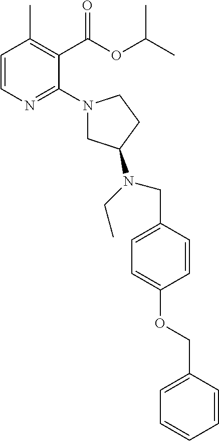 [SMILES: CCN(Cc1ccc(OCc2ccccc2)cc1)[C@@H]1CCN(c2nccc(C)c2C(=O)OC(C)C)C1] 1-methylethyl 2-{(3R)-3-[ethyl({4- [(phenylmethyl)oxy]phenyl}methyl)amino]- 1-pyrrolidinyl}-4-methyl-3-pyridinecarboxylate | 488.17 | 1.0 |
| Example 301 | 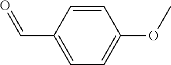 | 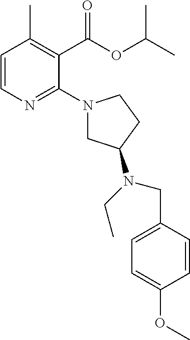 [SMILES: CCN(Cc1ccc(OC)cc1)[C@@H]1CCN(c2nccc(C)c2C(=O)OC(C)C)C1] 1-methylethyl 2-[(3R)-3-(ethyl{[4- (methyloxy)phenyl]methyl}amino)- 1-pyrrolidinyl]-4-methyl-3-pyridinecarboxylate | 412.14 | 0.8 |
| Example 302 | 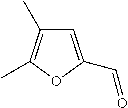 | 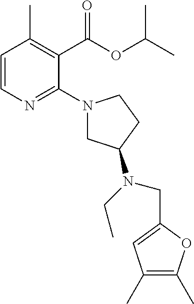 [SMILES: CCN(Cc1cc(C)c(C)o1)[C@@H]1CCN(c2nccc(C)c2C(=O)OC(C)C)C1] 1-methylethyl 2-{(3R)-3-[[(4,5-dimethyl-2- furanyl)methyl](ethyl)amino]-1- pyrrolidinyl}-4-methyl-3-pyridinecarboxylate | 400.12 | 0.8 |
| Example 303 | 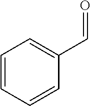 | 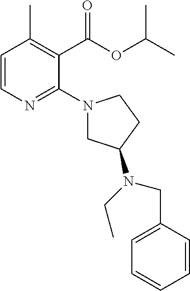 [SMILES: CCN(Cc1ccccc1)[C@@H]1CCN(c2nccc(C)c2C(=O)OC(C)C)C1] 1-methylethyl 2-{(3R)-3-[ethyl(phenylmethyl)amino]-1- pyrrolidinyl}-4-methyl-3-pyridinecarboxylate | 382.15 | 0.8 |
| Example 304 | 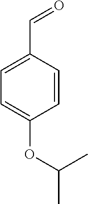 | 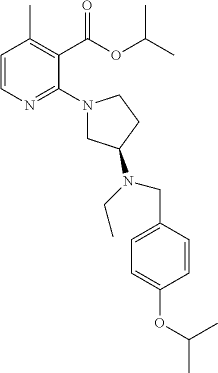 [SMILES: CCN(Cc1ccc(OC(C)C)cc1)[C@@H]1CCN(c2nccc(C)c2C(=O)OC(C)C)C1] 1-methylethyl 2-{(3R)-3-[ethyl({4-[(1- methylethyl)oxy]phenyl}methyl)amino]- 1-pyrrolidinyl}-4-methyl-3-pyridinecarboxylate | 440.17 | 0.9 |

### Table 16

Following the procedure as described above in the preparation of 1-methylethyl 2-{methyl[(3R)-1-({2-[(trifluoromethyl)oxy]phenyl}methyl)-3-pyrrolidinyl]amino}-3-pyridinecarboxylate, 1-methylethyl 2-{methyl[(3R)-3-pyrrolidinyl]amino}-3-pyridinecarboxylate (25 mg, 0.095 mmol) was reacted with the appropriate aldehyde or ketone to yield the examples listed in Table XVI.

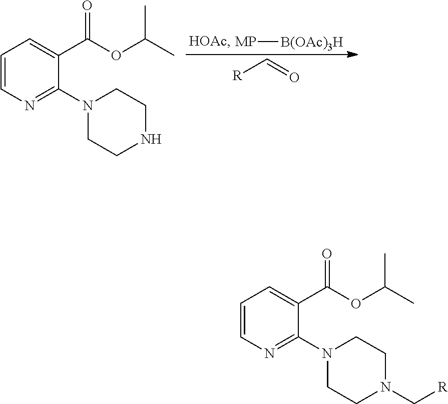

TABLE XVI

| | | | LC-MS | |
|---|---|---|---|---|
| | Aldehyde | | m/z | RT |
| Example | (ketone) | Product Name | (M + H)⁺ | (min) |
| Example 305 | 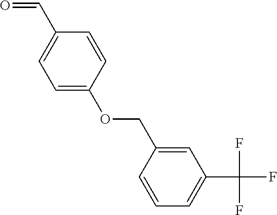 [SMILES: O=Cc1ccc(OCc2cccc(C(F)(F)F)c2)cc1] | 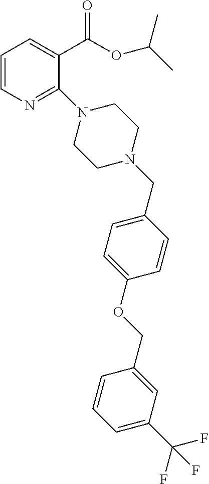 [SMILES: CC(C)OC(=O)c1cccnc1N1CCN(Cc2ccc(OCc3cccc(C(F)(F)F)c3)cc2)CC1] 1-methylethyl 2-(4-{[4-({[3- (trifluoromethyl)phenyl]methyl}oxy) phenyl]methyl}-1-piperazinyl)- 3-pyridinecarboxylate | 514.3 | 1.13 |
| Example 306 | 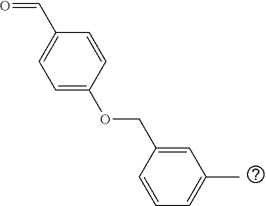 [SMILES: Cc1cccc(COc2ccc(C=O)cc2)c1] | 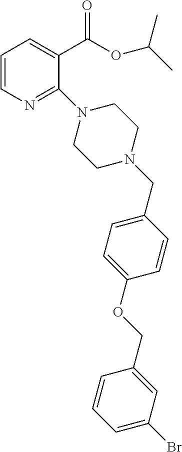 [SMILES: CC(C)OC(=O)c1cccnc1N1CCN(Cc2ccc(OCc3cccc(Br)c3)cc2)CC1] 1-methylethyl 2-{4-[(4-{[(3- bromophenyl)methyl]oxy}phenyl) methyl]-1-piperazinyl}-3-pyridinecarboxylate | 524.3 | 1.1 |
| Example 307 | 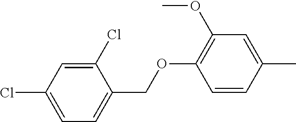 [SMILES: COc1cc(C)ccc1OCc1ccc(Cl)cc1Cl] | 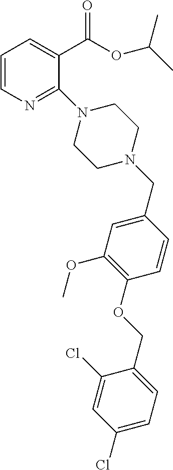 [SMILES: COc1cc(CN2CCN(c3ncccc3C(=O)OC(C)C)CC2)ccc1OCc1ccc(Cl)cc1Cl] 1-methylethyl 2-(4-{[4-{[(2,4- dichlorophenyl)methyl]oxy}-3- (methyloxy)phenyl]methyl}-1-piperazinyl)-3- pyridinecarboxylate | 544.2 | 1.2 |
| Example 308 | 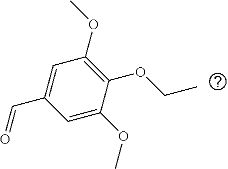 | 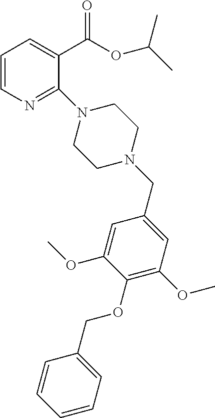 [SMILES: COc1cc(CN2CCN(c3ncccc3C(=O)OC(C)C)CC2)cc(OC)c1OCc1ccccc1] 1-methylethyl 2-[4-({3,5-bis(methyloxy)-4- [(phenylmethyl)oxy]phenyl}methyl)- 1-piperazinyl]-3-pyridinecarboxylate | 506.3 | 1.2 |
| Example 309 | 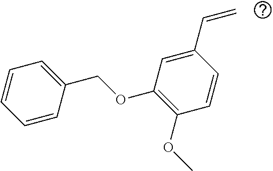 [SMILES: C=Cc1ccc(OC)c(OCc2ccccc2)c1] | 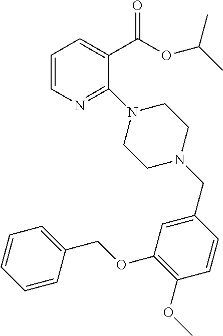 [SMILES: COc1ccc(CN2CCN(c3ncccc3C(=O)OC(C)C)CC2)cc1OCc1ccccc1] 1-methylethyl 2-[4-({4-(methyloxy)-3- [(phenylmethyl)oxy]phenyl}methyl)- 1-piperazinyl]-3-pyridinecarboxylate | 476.3 | 1.0 |
| Example 310 | 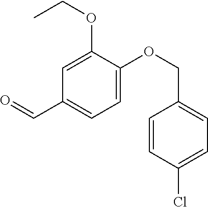 [SMILES: CCOc1cc(C=O)ccc1OCc1ccc(Cl)cc1] | 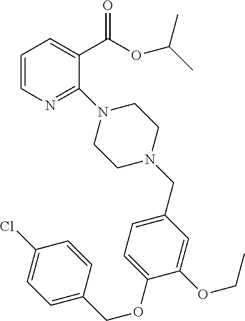 [SMILES: CCOc1cc(CN2CCN(c3ncccc3C(=O)OC(C)C)CC2)ccc1OCc1ccc(Cl)cc1] 1-methylethyl 2-(4-{[4-{[(4-chlorophenyl)methyl]oxy}-3- (ethyloxy)phenyl]methyl}-1-piperazinyl)-3- pyridinecarboxylate | 524.3 | 1.1 |
| Example 311 | 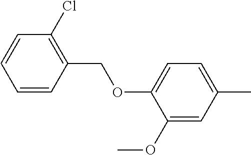 [SMILES: COc1cc(C)ccc1OCc1ccccc1Cl] | 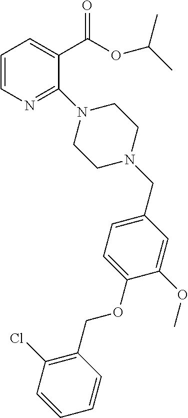 [SMILES: COc1cc(CN2CCN(c3ncccc3C(=O)OC(C)C)CC2)ccc1OCc1ccccc1Cl] 1-methylethyl 2-(4-{[4-{[(2- chlorophenyl)methyl]oxy}-3- (methyloxy)phenyl]methyl}-1- piperazinyl)-3-pyridinecarboxylate | 510.1 | 1.1 |
| Example 312 | 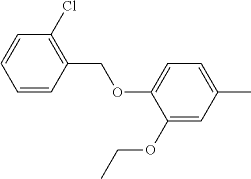 [SMILES: CCOc1cc(C)ccc1OCc1ccccc1Cl] | 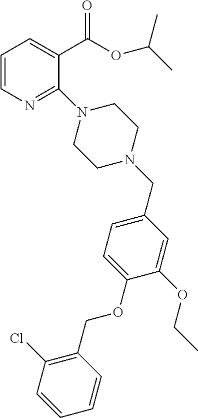 [SMILES: CCOc1cc(CN2CCN(c3ncccc3C(=O)OC(C)C)CC2)ccc1OCc1ccccc1Cl] 1-methylethyl 2-(4-{[4-{[(2- chlorophenyl)methyl]oxy}-3- (ethyloxy)phenyl]methyl}-1- piperazinyl)-3-pyridinecarboxylate | 524.3 | 1.1 |
| Example 313 | 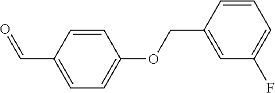 [SMILES: O=Cc1ccc(OCc2cccc(F)c2)cc1] | 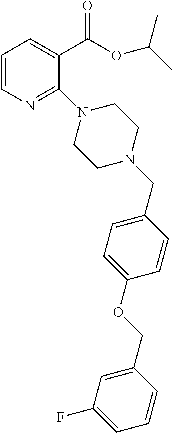 [SMILES: CC(C)OC(=O)c1cccnc1N1CCN(Cc2ccc(OCc3cccc(F)c3)cc2)CC1] 1-methylethyl 2-{4-[(4-{[(3- fluorophenyl)methyl]oxy}phenyl) methyl]-1-piperazinyl}-3-pyridinecarboxylate | 464.5 | 1.1 |
| Example 314 | 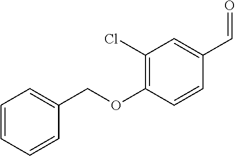 [SMILES: O=Cc1ccc(OCc2ccccc2)c(Cl)c1] | 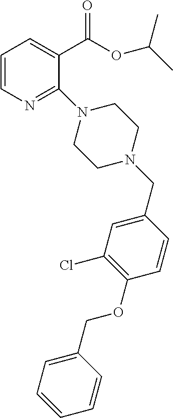 [SMILES: CC(C)OC(=O)c1cccnc1N1CCN(Cc2ccc(OCc3ccccc3)c(Cl)c2)CC1] 1-methylethyl 2-[4-({3-chloro-4- [(phenylmethyl)oxy]phenyl}methyl)- 1-piperazinyl]-3-pyridinecarboxylate | 480.1 | 1.1 |
| Example 315 | 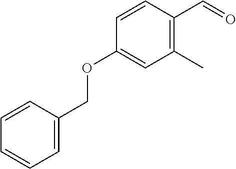 [SMILES: Cc1cc(OCc2ccccc2)ccc1C=O] | 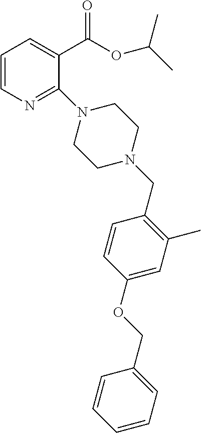 [SMILES: Cc1cc(OCc2ccccc2)ccc1CN1CCN(c2ncccc2C(=O)OC(C)C)CC1] 1-methylethyl 2-[4-({2-methyl-4- [(phenylmethyl)oxy]phenyl}methyl)- 1-piperazinyl]-3-pyridinecarboxylate | 460.2 | 1.1 |
| Example 316 | 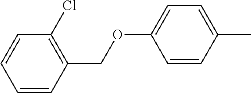 [SMILES: Cc1ccc(OCc2ccccc2Cl)cc1] | 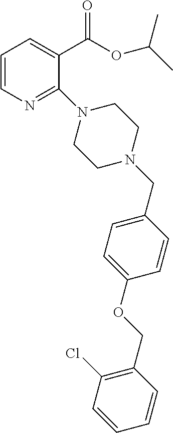 [SMILES: CC(C)OC(=O)c1cccnc1N1CCN(Cc2ccc(OCc3ccccc3Cl)cc2)CC1] 1-methylethyl 2-{4-[(4-{[(2- chlorophenyl)methyl]oxy}phenyl) methyl]-1-piperazinyl}-3-pyridinecarboxylate | 480.1 | 1.1 |
| Example 317 | 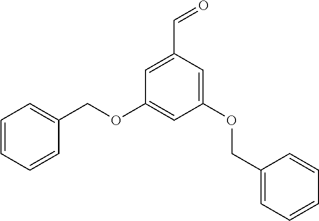 [SMILES: O=Cc1cc(OCc2ccccc2)cc(OCc2ccccc2)c1] | 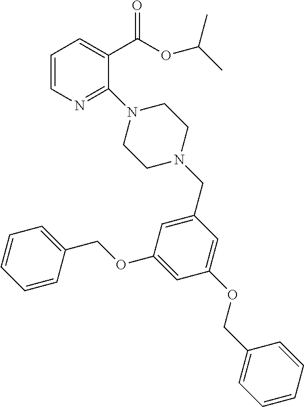 [SMILES: CC(C)OC(=O)c1cccnc1N1CCN(Cc2cc(OCc3ccccc3)cc(OCc3ccccc3)c2)CC1] 1-methylethyl 2-[4-({3,5- bis[(phenylmethyl)oxy]phenyl}methyl)- 1-piperazinyl]-3-pyridinecarboxylate | 552.6 | 1.2 |
| Example 318 | 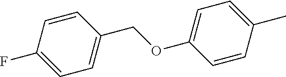 [SMILES: Cc1ccc(OCc2ccc(F)cc2)cc1] | 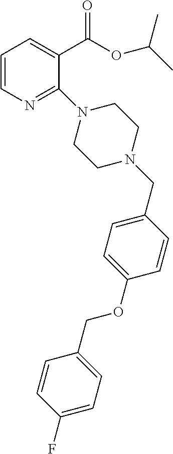 [SMILES: CC(C)OC(=O)c1cccnc1N1CCN(Cc2ccc(OCc3ccc(F)cc3)cc2)CC1] 1-methylethyl 2-{4-[(4-{[(4- fluorophenyl)methyl]oxy}phenyl) methyl]-1-piperazinyl}-3-pyridinecarboxylate | 464.2 | 1.1 |
| Example 319 | 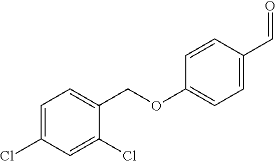 [SMILES: O=Cc1ccc(OCc2ccc(Cl)cc2Cl)cc1] | 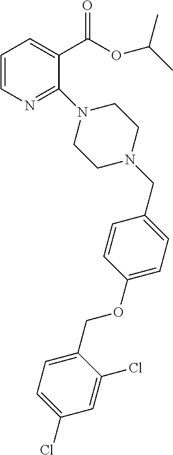 [SMILES: CC(C)OC(=O)c1cccnc1N1CCN(Cc2ccc(OCc3ccc(Cl)cc3Cl)cc2)CC1] 1-methylethyl 2-{4-[(4-{[(2,4- dichlorophenyl)methyl]oxy}phenyl) methyl]-1-piperazinyl}-3-pyridinecarboxylate | 514.3 | 1.2 |
| Example 320 | 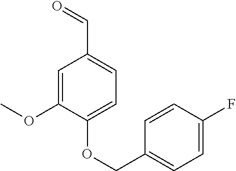 [SMILES: COc1cc(C=O)ccc1OCc1ccc(F)cc1] | 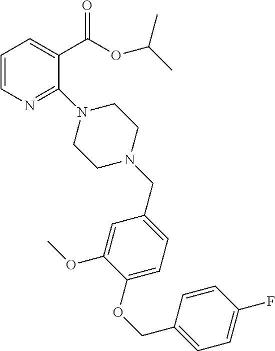 [SMILES: COc1cc(CN2CCN(c3ncccc3C(=O)OC(C)C)CC2)ccc1OCc1ccc(F)cc1] 1-methylethyl 2-(4-{[4-{[(4- fluorophenyl)methyl]oxy}-3- (methyloxy)phenyl]methyl}-1- piperazinyl)-3-pyridinecarboxylate | 494.2 | 1.1 |
| Example 321 | 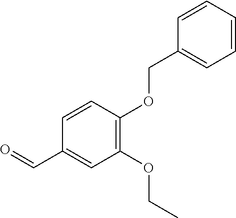 [SMILES: CCOc1cc(C=O)ccc1OCc1ccccc1] | 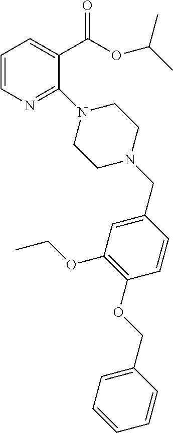 [SMILES: CCOc1cc(CN2CCN(c3ncccc3C(=O)OC(C)C)CC2)ccc1OCc1ccccc1] 1-methylethyl 2-[4-({3-(ethyloxy)-4- [(phenylmethyl)oxy]phenyl}methyl)- 1-piperazinyl]-3-pyridinecarboxylate | 490.3 | 1.1 |
| Example 322 | 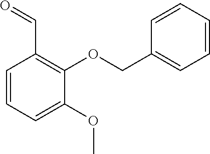 [SMILES: COc1cccc(C=O)c1OCc1ccccc1] | 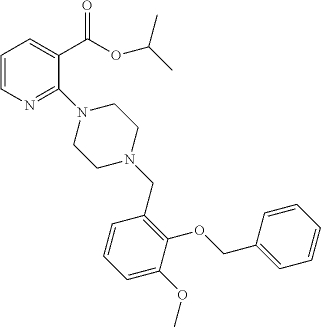 [SMILES: COc1cccc(CN2CCN(c3ncccc3C(=O)OC(C)C)CC2)c1OCc1ccccc1] 1-methylethyl 2-[4-({3-(methyloxy)-2- [(phenylmethyl)oxy]phenyl}methyl)- 1-piperazinyl]-3-pyridinecarboxylate | 476.2 | 1.1 |
| Example 323 | 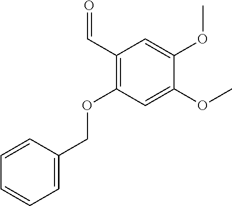 [SMILES: COc1cc(C=O)c(OCc2ccccc2)cc1OC] | 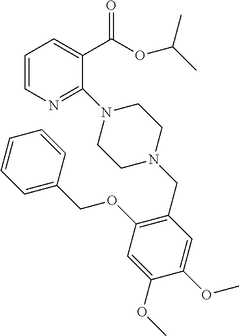 [SMILES: COc1cc(CN2CCN(c3ncccc3C(=O)OC(C)C)CC2)c(OCc2ccccc2)cc1OC] 1-methylethyl 2-[4-({4,5-bis(methyloxy)-2- [(phenylmethyl)oxy]phenyl}methyl)- 1-piperazinyl]-3-pyridinecarboxylate | 506.2 | 1.1 |
| Example 324 | 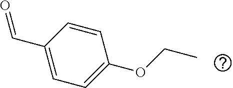 | 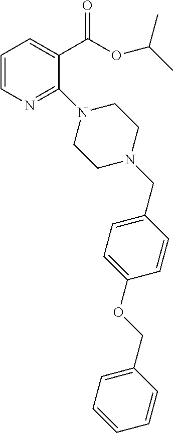 [SMILES: CC(C)OC(=O)c1cccnc1N1CCN(Cc2ccc(OCc3ccccc3)cc2)CC1] 1-methylethyl 2-[4-({4- [(phenylmethyl)oxy]phenyl}methyl)- 1-piperazinyl]-3-pyridinecarboxylate | 446.5 | 1.1 |
| Example 325 | 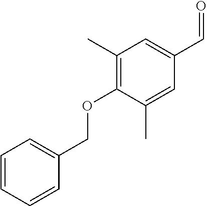 [SMILES: Cc1cc(C=O)cc(C)c1OCc1ccccc1] | 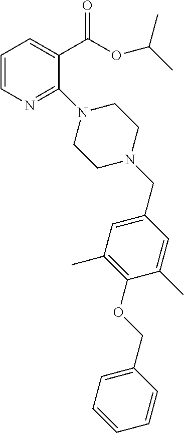 [SMILES: Cc1cc(CN2CCN(c3ncccc3C(=O)OC(C)C)CC2)cc(C)c1OCc1ccccc1] 1-methylethyl 2-[4-({3,5-dimethyl-4- [(phenylmethyl)oxy]phenyl}methyl)- 1-piperazinyl]-3-pyridinecarboxylate | 474.3 | 1.1 |
| Example 326 | 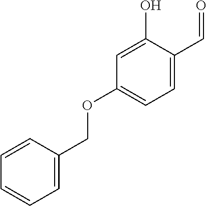 [SMILES: O=Cc1ccc(OCc2ccccc2)cc1O] | 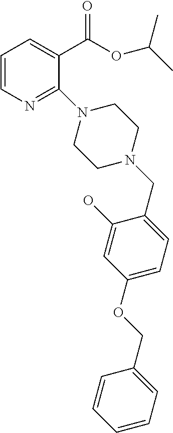 [SMILES: CC(C)OC(=O)c1cccnc1N1CCN(Cc2ccc(OCc3ccccc3)cc2O)CC1] 1-methylethyl 2-[4-({2-hydroxy-4- [(phenylmethyl)oxy]phenyl}methyl)- 1-piperazinyl]-3-pyridinecarboxylate | 462.1 | 1.0 |
| Example 327 | 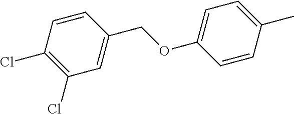 [SMILES: Cc1ccc(OCc2ccc(Cl)c(Cl)c2)cc1] | 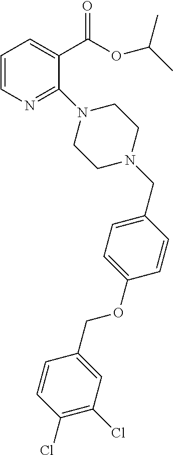 [SMILES: CC(C)OC(=O)c1cccnc1N1CCN(Cc2ccc(OCc3ccc(Cl)c(Cl)c3)cc2)CC1] 1-methylethyl 2-{4-[(4-{[(3,4- dichlorophenyl)methyl]oxy}phenyl) methyl]-1-piperazinyl}-3-pyridinecarboxylate | 514.3 | 1.2 |
| Example 328 | 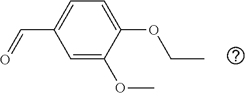 | 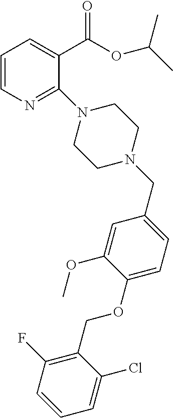 [SMILES: COc1cc(CN2CCN(c3ncccc3C(=O)OC(C)C)CC2)ccc1OCc1c(F)cccc1Cl] 1-methylethyl 2-(4-{[4-{[(2-chloro- 6-fluorophenyl)methyl]oxy}-3- (methyloxy)phenyl]methyl}-1- piperazinyl)-3-pyridinecarboxylate | 528.3 | 1.1 |
| Example 329 | 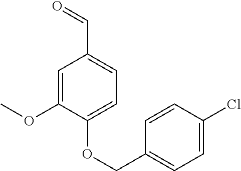 [SMILES: COc1cc(C=O)ccc1OCc1ccc(Cl)cc1] | 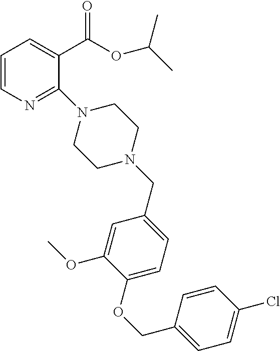 [SMILES: COc1cc(CN2CCN(c3ncccc3C(=O)OC(C)C)CC2)ccc1OCc1ccc(Cl)cc1] 1-methylethyl 2-(4-{[4-{[(4- chlorophenyl)methyl]oxy}-3- (methyloxy)phenyl]methyl}-1- piperazinyl)-3-pyridinecarboxylate | 510.1 | 1.1 |
| Example 330 | 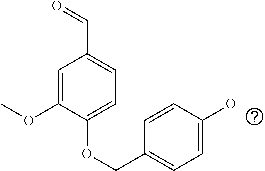 [SMILES: COc1cc(C=O)ccc1OCc1ccc(O)cc1] | 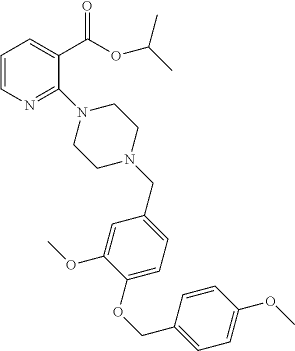 [SMILES: COc1ccc(COc2ccc(CN3CCN(c4ncccc4C(=O)OC(C)C)CC3)cc2OC)cc1] 1-methylethyl 2-(4-{[3-(methyloxy)-4-({[4- (methyloxy)phenyl]methyl}oxy)phe- nyl]methyl}-1-piperazinyl)-3-pyridinecarboxylate | 506.3 | 1.0 |
| Example 331 | 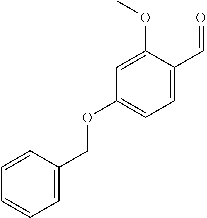 [SMILES: COc1cc(OCc2ccccc2)ccc1C=O] | 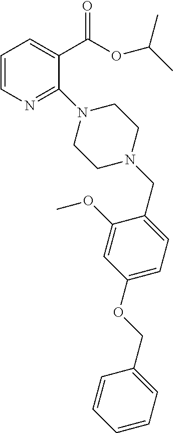 [SMILES: COc1cc(OCc2ccccc2)ccc1CN1CCN(c2ncccc2C(=O)OC(C)C)CC1] 1-methylethyl 2-[4-({2-(methyloxy)-4- [(phenylmethyl)oxy]phenyl}methyl)- 1-piperazinyl]-3-pyridinecarboxylate | 476.1 | 1.1 |
| Example 332 | 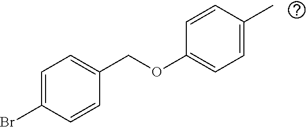 [SMILES: Cc1ccc(OCc2ccc(Br)cc2)cc1] | 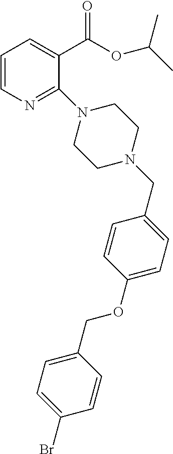 [SMILES: CC(C)OC(=O)c1cccnc1N1CCN(Cc2ccc(OCc3ccc(Br)cc3)cc2)CC1] 1-methylethyl 2-{4-[(4-{[(4- bromophenyl)methyl]oxy}phenyl) methyl]-1-piperazinyl}-3-pyridinecarboxylate | 524.2 | 1.1 |
| Example 333 | 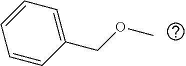 | 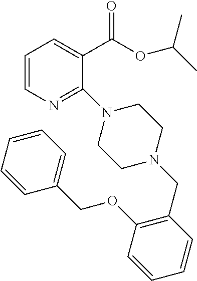 [SMILES: CC(C)OC(=O)c1cccnc1N1CCN(Cc2ccccc2OCc2ccccc2)CC1] 1-methylethyl 2-[4-({2- [(phenylmethyl)oxy]phenyl}methyl)- 1-piperazinyl]-3-pyridinecarboxylate | 446.5 | 1.1 |
| Example 334 | 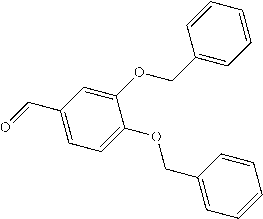 [SMILES: O=Cc1ccc(OCc2ccccc2)c(OCc2ccccc2)c1] | 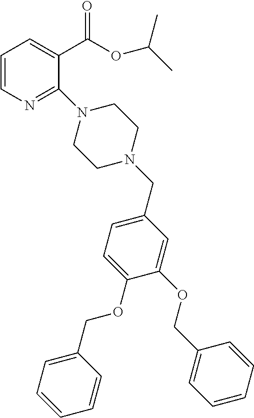 [SMILES: CC(C)OC(=O)c1cccnc1N1CCN(Cc2ccc(OCc3ccccc3)c(OCc3ccccc3)c2)CC1] 1-methylethyl 2-[4-({3,4- bis[(phenylmethyl)oxy]phenyl}methyl)- 1-piperazinyl]-3-pyridinecarboxylate | 552.6 | 1.2 |
| Example 335 | 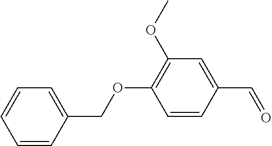 [SMILES: COc1cc(C=O)ccc1OCc1ccccc1] | 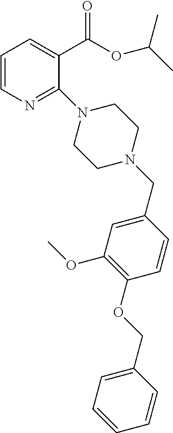 [SMILES: COc1cc(CN2CCN(c3ncccc3C(=O)OC(C)C)CC2)ccc1OCc1ccccc1] 1-methylethyl 2-[4-({3-(methyloxy)-4- [(phenylmethyl)oxy]phenyl}methyl)- 1-piperazinyl]-3-pyridinecarboxylate | 476.1 | 1.1 |
| Example 336 | 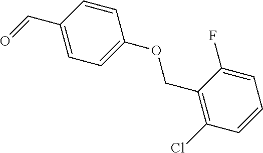 [SMILES: O=Cc1ccc(OCc2c(F)cccc2Cl)cc1] | 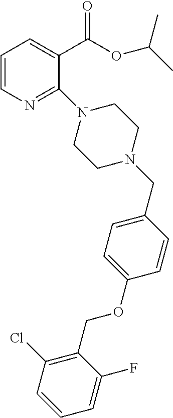 [SMILES: CC(C)OC(=O)c1cccnc1N1CCN(Cc2ccc(OCc3c(F)cccc3Cl)cc2)CC1] 1-methylethyl 2-{4-[(4-{[(2-chloro-6- fluorophenyl)methyl]oxy}phenyl) methyl]-1-piperazinyl}-3-pyridinecarboxylate | 498.4 | 1.1 |

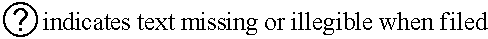

### Table 17

Following the procedure as described above in the preparation of 1-methylethyl 2-{methyl[(3R)-1-({2-[(trifluoromethyl)oxy]phenyl}methyl)-3-pyrrolidinyl]amino}-3-pyridinecarboxylate, 1-methylethyl 2-(1-piperazinyl)-3-pyridinecarboxylate (30 mg, 0.12 mmol) was reacted with the appropriate aldehyde or ketone to yield the examples listed in Table XVII.

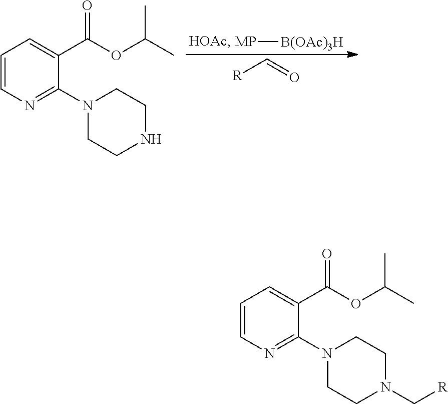

TABLE XVII

| | | | LC-MS | |
|---|---|---|---|---|
| | Aldehyde | | m/z | RT |
| Example | (ketone) | Product Name | (M + H)⁺ | (min) |
| Example 337 | 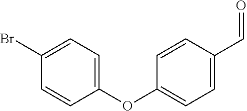 [SMILES: O=Cc1ccc(Oc2ccc(Br)cc2)cc1] | 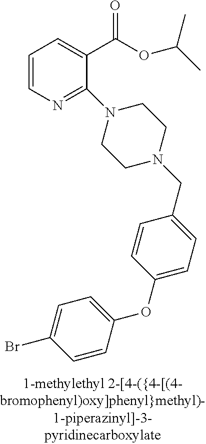 [SMILES: CC(C)OC(=O)c1cccnc1N1CCN(Cc2ccc(Oc3ccc(Br)cc3)cc2)CC1] | 512.2 | 1.1 |
| Example 338 | 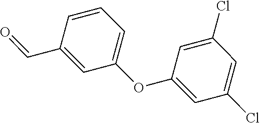 [SMILES: O=Cc1cccc(Oc2cc(Cl)cc(Cl)c2)c1] | 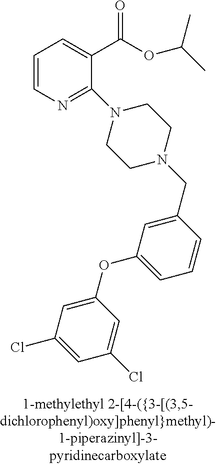 [SMILES: CC(C)OC(=O)c1cccnc1N1CCN(Cc2cccc(Oc3cc(Cl)cc(Cl)c3)c2)CC1] | 500.3 | 1.1 |
| Example 339 | 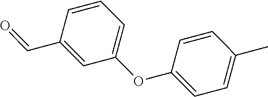 [SMILES: Cc1ccc(Oc2cccc(C=O)c2)cc1] | 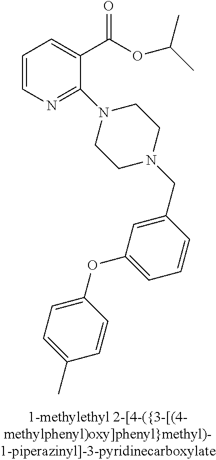 [SMILES: Cc1ccc(Oc2cccc(CN3CCN(c4ncccc4C(=O)OC(C)C)CC3)c2)cc1] | 446.5 | 1.1 |
| Example 340 | 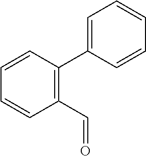 [SMILES: O=Cc1ccccc1-c1ccccc1] | 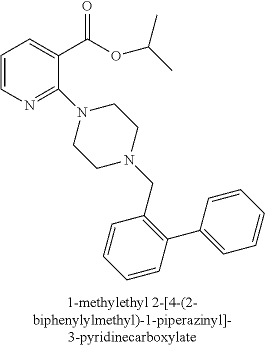 [SMILES: CC(C)OC(=O)c1cccnc1N1CCN(Cc2ccccc2-c2ccccc2)CC1] | 416.2 | 1.0 |
| Example 341 | 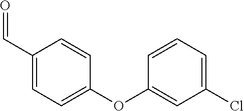 [SMILES: O=Cc1ccc(Oc2cccc(Cl)c2)cc1] | 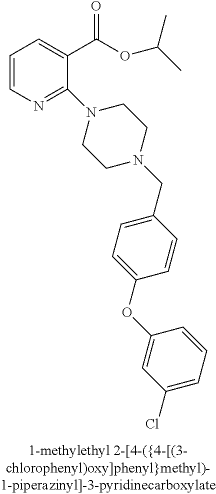 [SMILES: CC(C)OC(=O)c1cccnc1N1CCN(Cc2ccc(Oc3cccc(Cl)c3)cc2)CC1] | 466.2 | 1.1 |
| Example 342 | 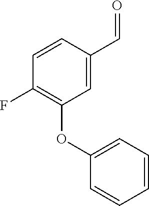 [SMILES: O=Cc1ccc(F)c(Oc2ccccc2)c1] | 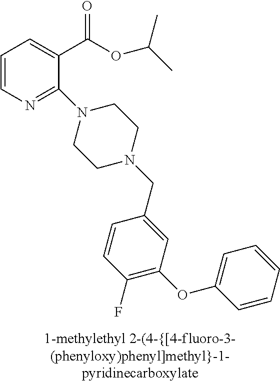 [SMILES: CC(C)OC(=O)c1cccnc1N1CCN(Cc2ccc(F)c(Oc3ccccc3)c2)CC1] | 450.0 | 1.0 |
| Example 343 | 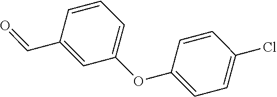 [SMILES: O=Cc1cccc(Oc2ccc(Cl)cc2)c1] | 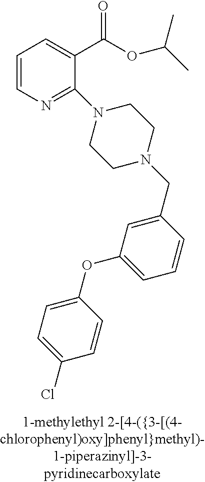 [SMILES: CC(C)OC(=O)c1cccnc1N1CCN(Cc2cccc(Oc3ccc(Cl)cc3)c2)CC1] | 466.0 | 1.1 |
| Example 344 | 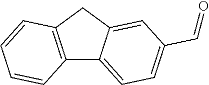 [SMILES: O=Cc1ccc2c(c1)Cc1ccccc1-2] | 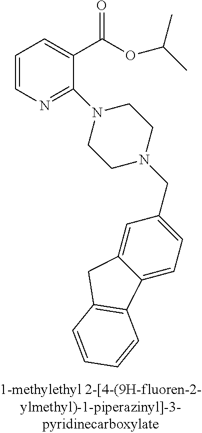 [SMILES: CC(C)OC(=O)c1cccnc1N1CCN(Cc2ccc3c(c2)Cc2ccccc2-3)CC1] | 427.9 | 1.0 |
| Example 345 | 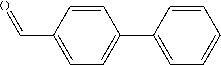 [SMILES: O=Cc1ccc(-c2ccccc2)cc1] | 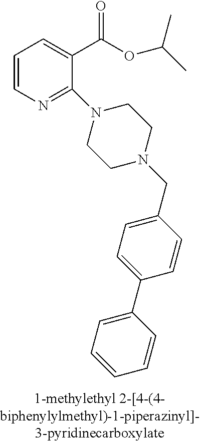 [SMILES: CC(C)OC(=O)c1cccnc1N1CCN(Cc2ccc(-c3ccccc3)cc2)CC1] | 416.2 | 1.0 |
| Example 346 | 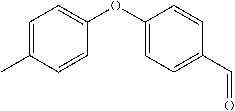 [SMILES: Cc1ccc(Oc2ccc(C=O)cc2)cc1] | 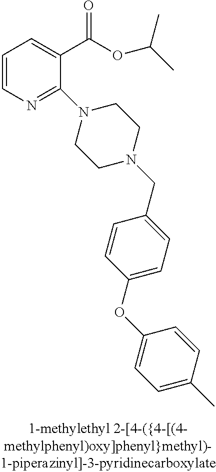 [SMILES: Cc1ccc(Oc2ccc(CN3CCN(c4ncccc4C(=O)OC(C)C)CC3)cc2)cc1] | 446.4 | 1.1 |
| Example 347 | 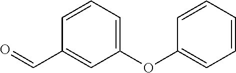 [SMILES: O=Cc1cccc(Oc2ccccc2)c1] | 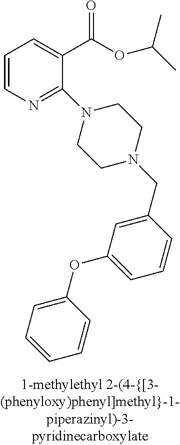 [SMILES: CC(C)OC(=O)c1cccnc1N1CCN(Cc2cccc(Oc3ccccc3)c2)CC1] | 432.0 | 1.0 |
| Example 348 | 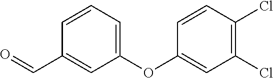 [SMILES: O=Cc1cccc(Oc2ccc(Cl)c(Cl)c2)c1] | 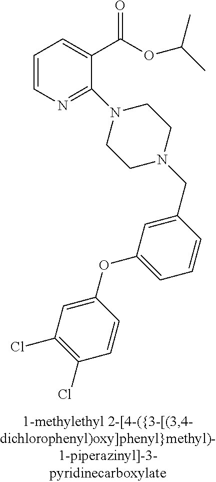 [SMILES: CC(C)OC(=O)c1cccnc1N1CCN(Cc2cccc(Oc3ccc(Cl)c(Cl)c3)c2)CC1] | 500.3 | 1.1 |
| Example 349 | 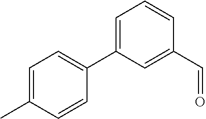 | 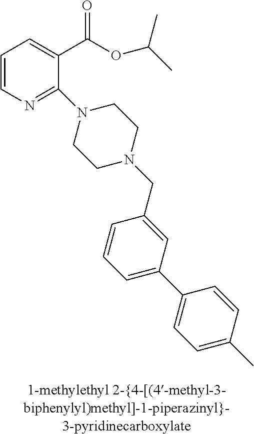 [SMILES: Cc1ccc(-c2cccc(CN3CCN(c4ncccc4C(=O)OC(C)C)CC3)c2)cc1] | 430.0 | 1.1 |
| Example 350 | 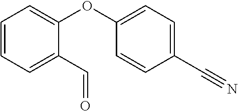 [SMILES: N#Cc1ccc(Oc2ccccc2C=O)cc1] | 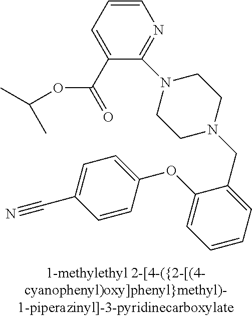 [SMILES: CC(C)OC(=O)c1cccnc1N1CCN(Cc2ccccc2Oc2ccc(C#N)cc2)CC1] | 456.9 | 1.0 |
| Example 351 | 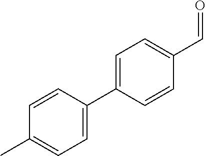 | 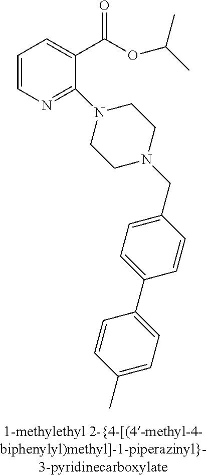 [SMILES: Cc1ccc(-c2ccc(CN3CCN(c4ncccc4C(=O)OC(C)C)CC3)cc2)cc1] | 430.0 | 1.1 |
| Example 352 | 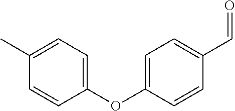 [SMILES: Cc1ccc(Oc2ccc(C=O)cc2)cc1] | 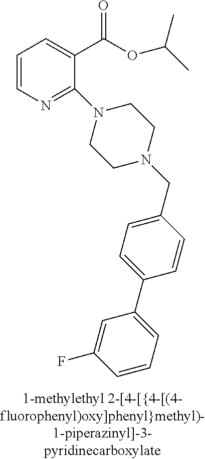 [SMILES: CC(C)OC(=O)c1cccnc1N1CCN(Cc2ccc(-c3cccc(F)c3)cc2)CC1] | 450.0 | 1.0 |
| Example 353 | 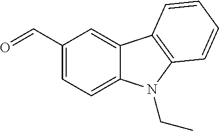 [SMILES: CCn1c2ccccc2c2cc(C=O)ccc21] | 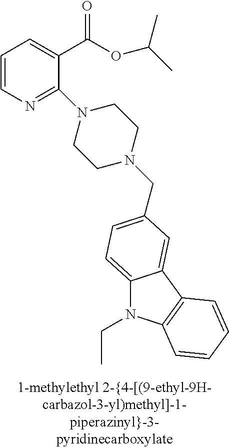 [SMILES: CCn1c2ccccc2c2cc(CN3CCN(c4ncccc4C(=O)OC(C)C)CC3)ccc21] | 457.0 | 1.1 |
| Example 354 | 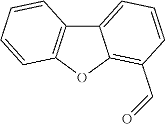 [SMILES: O=Cc1cccc2c1oc1ccccc12] | 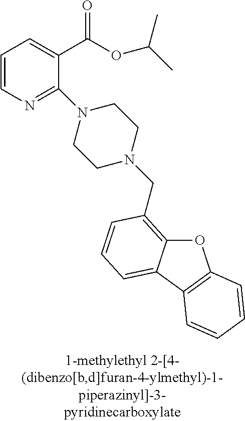 [SMILES: CC(C)OC(=O)c1cccnc1N1CCN(Cc2cccc3c2oc2ccccc23)CC1] | 429.9 | 1.0 |
| Example 355 | 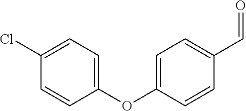 [SMILES: O=Cc1ccc(Oc2ccc(Cl)cc2)cc1] | 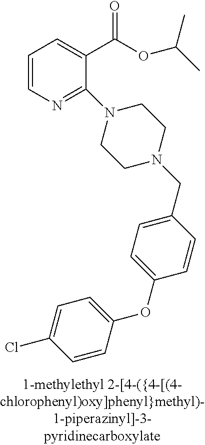 [SMILES: CC(C)OC(=O)c1cccnc1N1CCN(Cc2ccc(Oc3ccc(Cl)cc3)cc2)CC1] | 466.1 | 1.1 |
| Example 356 | 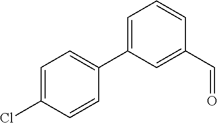 [SMILES: O=Cc1cccc(-c2ccc(Cl)cc2)c1] | 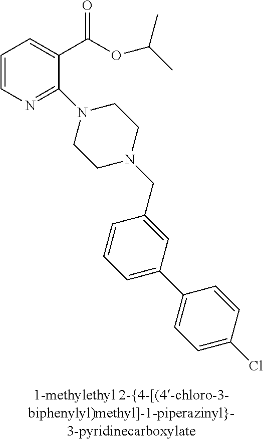 [SMILES: CC(C)OC(=O)c1cccnc1N1CCN(Cc2cccc(-c3ccc(Cl)cc3)c2)CC1] | 450.0 | 1.1 |
| Example 357 | 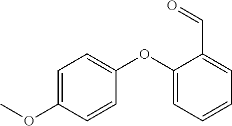 [SMILES: COc1ccc(Oc2ccccc2C=O)cc1] | 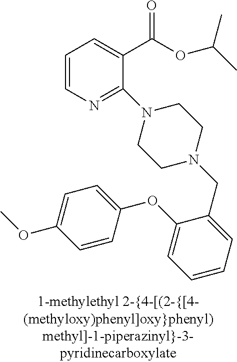 [SMILES: COc1ccc(Oc2ccccc2CN2CCN(c3ncccc3C(=O)OC(C)C)CC2)cc1] | 462.1 | 1.0 |
| Example 358 | 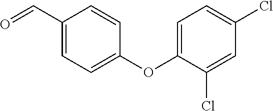 [SMILES: O=Cc1ccc(Oc2ccc(Cl)cc2Cl)cc1] | 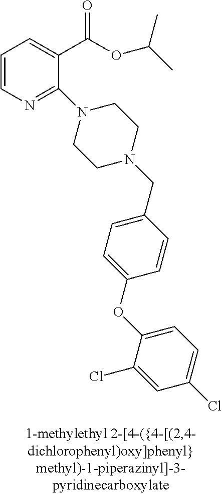 [SMILES: CC(C)OC(=O)c1cccnc1N1CCN(Cc2ccc(Oc3ccc(Cl)cc3Cl)cc2)CC1] | 500.3 | 1.1 |
| Example 359 | 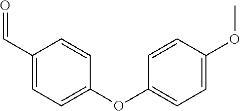 [SMILES: COc1ccc(Oc2ccc(C=O)cc2)cc1] | 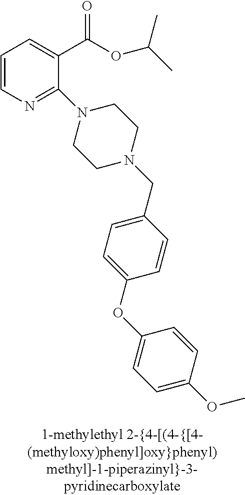 [SMILES: COc1ccc(Oc2ccc(CN3CCN(c4ncccc4C(=O)OC(C)C)CC3)cc2)cc1] | 462.1 | 1.0 |
| Example 360 | 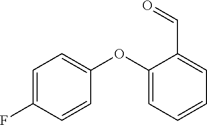 [SMILES: O=Cc1ccccc1Oc1ccc(F)cc1] | 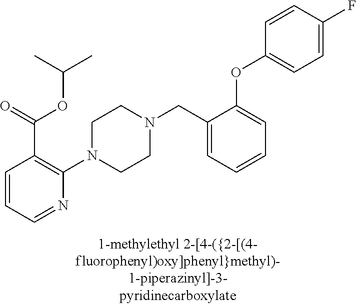 [SMILES: CC(C)OC(=O)c1cccnc1N1CCN(Cc2ccccc2Oc2ccc(F)cc2)CC1] | 449.9 | 1.0 |
| Example 361 | 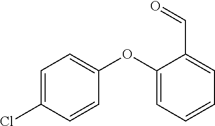 [SMILES: O=Cc1ccccc1Oc1ccc(Cl)cc1] | 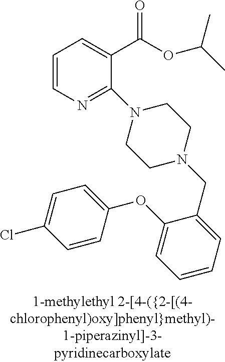 [SMILES: CC(C)OC(=O)c1cccnc1N1CCN(Cc2ccccc2Oc2ccc(Cl)cc2)CC1] | 466.2 | 1.1 |
| Example 362 | 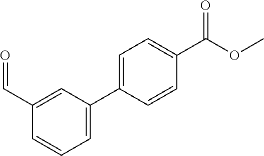 [SMILES: COC(=O)c1ccc(-c2cccc(C=O)c2)cc1] | 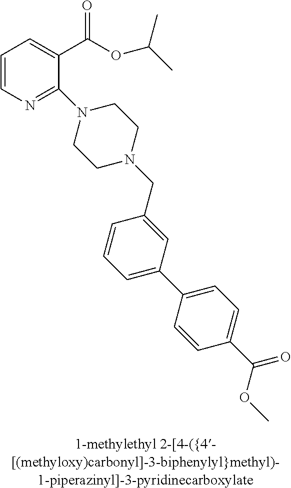 [SMILES: COC(=O)c1ccc(-c2cccc(CN3CCN(c4ncccc4C(=O)OC(C)C)CC3)c2)cc1] | 474.3 | 1.0 |
| Example 363 | 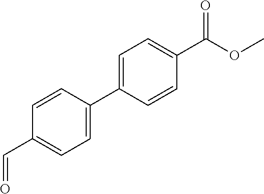 [SMILES: COC(=O)c1ccc(-c2ccc(C=O)cc2)cc1] | 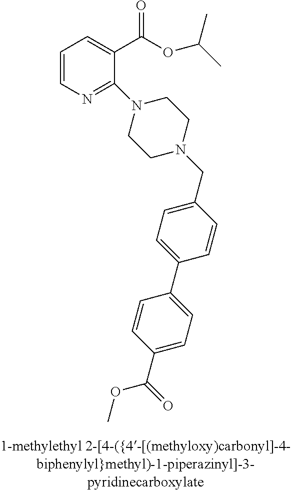 [SMILES: COC(=O)c1ccc(-c2ccc(CN3CCN(c4ncccc4C(=O)OC(C)C)CC3)cc2)cc1] | 474.3 | 1.0 |
| Example 364 | 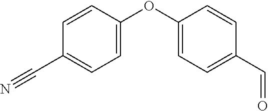 [SMILES: N#Cc1ccc(Oc2ccc(C=O)cc2)cc1] | 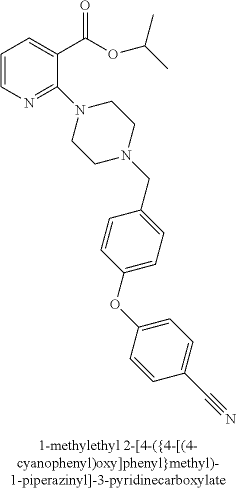 [SMILES: CC(C)OC(=O)c1cccnc1N1CCN(Cc2ccc(Oc3ccc(C#N)cc3)cc2)CC1] | 456.9 | 1.0 |
| Example 365 | 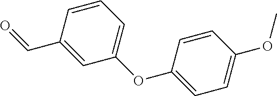 [SMILES: COc1ccc(Oc2cccc(C=O)c2)cc1] | 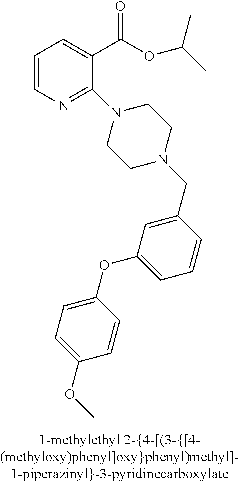 [SMILES: COc1ccc(Oc2cccc(CN3CCN(c4ncccc4C(=O)OC(C)C)CC3)c2)cc1] | 462.0 | 1.0 |
| Example 366 | 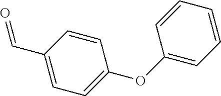 [SMILES: O=Cc1ccc(Oc2ccccc2)cc1] | 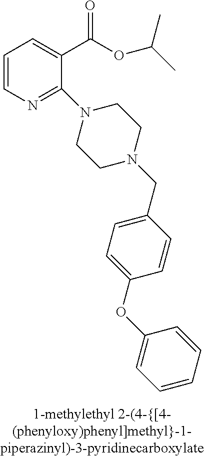 [SMILES: CC(C)OC(=O)c1cccnc1N1CCN(Cc2ccc(Oc3ccccc3)cc2)CC1] | 432.0 | 1.0 |
| Example 367 | 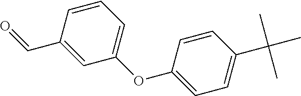 [SMILES: CC(C)(C)c1ccc(Oc2cccc(C=O)c2)cc1] | 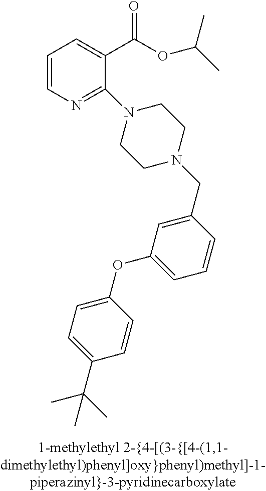 [SMILES: CC(C)OC(=O)c1cccnc1N1CCN(Cc2cccc(Oc3ccc(C(C)(C)C)cc3)c2)CC1] | 488.2 | 1.2 |
| Example 368 | 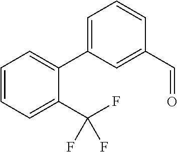 [SMILES: O=Cc1cccc(-c2ccccc2C(F)(F)F)c1] | 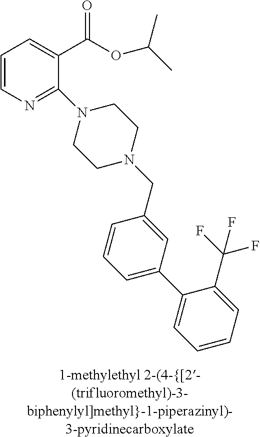 [SMILES: CC(C)OC(=O)c1cccnc1N1CCN(Cc2cccc(-c3ccccc3C(F)(F)F)c2)CC1] | 483.9 | 1.1 |
| Example 369 | 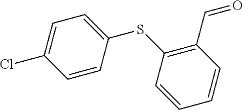 [SMILES: O=Cc1ccccc1Sc1ccc(Cl)cc1] | 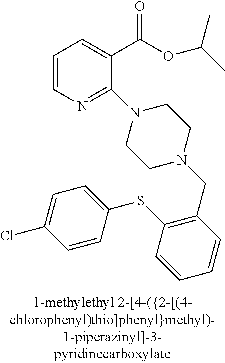 [SMILES: CC(C)OC(=O)c1cccnc1N1CCN(Cc2ccccc2Sc2ccc(Cl)cc2)CC1] | 481.9 | 1.1 |
| Example 370 | 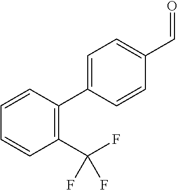 [SMILES: O=Cc1ccc(-c2ccccc2C(F)(F)F)cc1] | 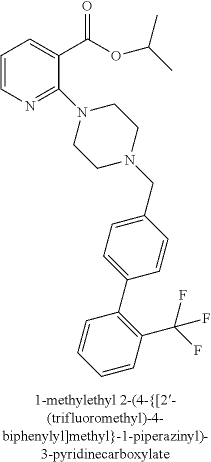 [SMILES: CC(C)OC(=O)c1cccnc1N1CCN(Cc2ccc(-c3ccccc3C(F)(F)F)cc2)CC1] | 484.0 | 1.1 |
| Example 371 | 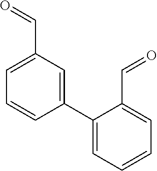 [SMILES: O=Cc1cccc(-c2ccccc2C=O)c1] | 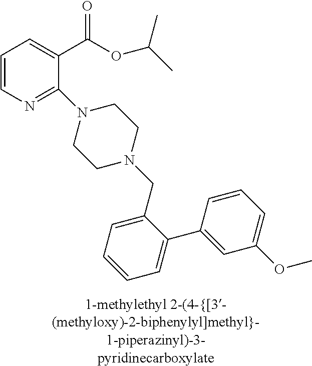 [SMILES: COc1cccc(-c2ccccc2CN2CCN(c3ncccc3C(=O)OC(C)C)CC2)c1] | 446.4 | 1.0 |
| Example 372 | 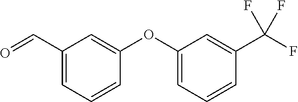 [SMILES: O=Cc1cccc(Oc2cccc(C(F)(F)F)c2)c1] | 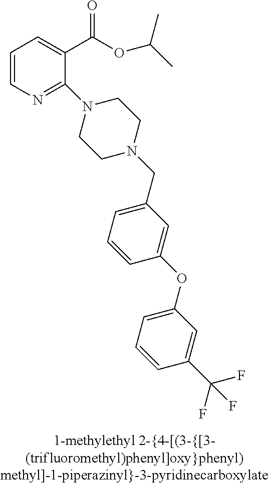 [SMILES: CC(C)OC(=O)c1cccnc1N1CCN(Cc2cccc(Oc3cccc(C(F)(F)F)c3)c2)CC1] | 500.3 | 1.1 |
| Example 373 | 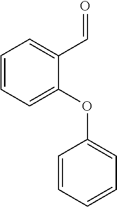 [SMILES: O=Cc1ccccc1Oc1ccccc1] | 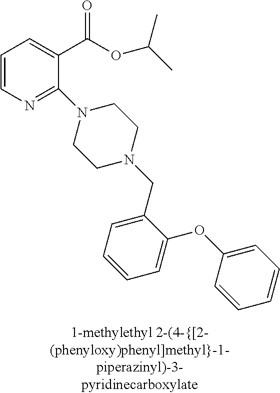 [SMILES: CC(C)OC(=O)c1cccnc1N1CCN(Cc2ccccc2Oc2ccccc2)CC1] | 432.0 | 1.0 |

### Table 18

Following the procedure as described above in the preparation of 1-methylethyl 2-{methyl[(3R)-1-({2-[(trifluoromethyl)oxy]phenyl}methyl)-3-pyrrolidinyl]amino}-3-pyridinecarboxylate, 1-methylethyl 2-{methyl[(3S)-3-pyrrolidinyl]amino}-3-pyridinecarboxylate (20 mg, 0.076 mmol) was reacted with the appropriate aldehyde or ketone to yield the examples listed in Table XVIII.

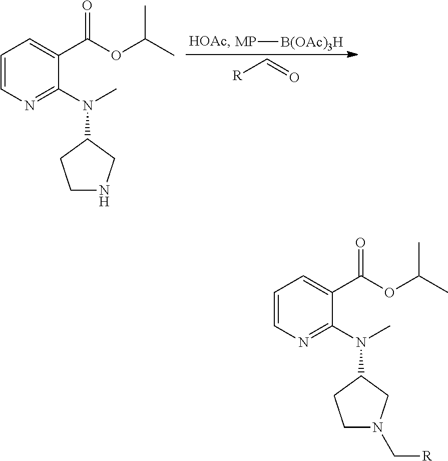

TABLE XVIII

| | | | LC-MS | |
|---|---|---|---|---|
| | | | m/z | RT |
| Example | Aldehyde | Product Name | (M + H)⁺ | (min) |
| Example 374 | 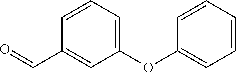 [SMILES: O=Cc1cccc(Oc2ccccc2)c1] | 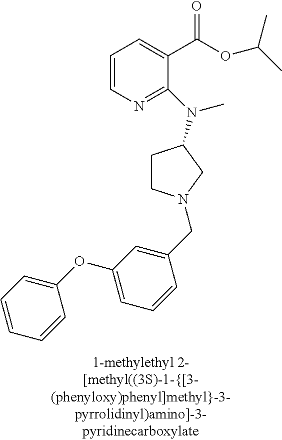 [SMILES: CC(C)OC(=O)c1cccnc1N(C)[C@H]1CCN(Cc2cccc(Oc3ccccc3)c2)C1] | 446.11 | 1.1 |
| Example 375 | 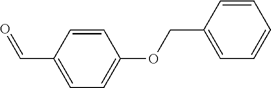 [SMILES: O=Cc1ccc(OCc2ccccc2)cc1] | 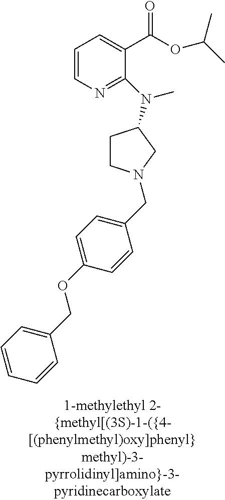 [SMILES: CC(C)OC(=O)c1cccnc1N(C)[C@H]1CCN(Cc2ccc(OCc3ccccc3)cc2)C1] | 460 | 1.1 |
| Example 376 | 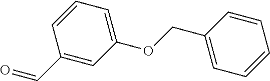 [SMILES: O=Cc1cccc(OCc2ccccc2)c1] | 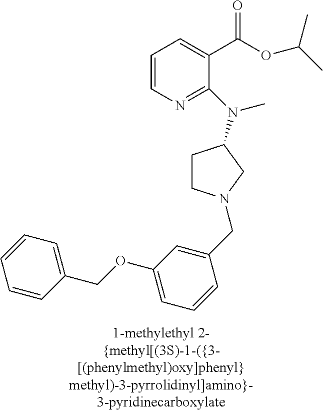 [SMILES: CC(C)OC(=O)c1cccnc1N(C)[C@H]1CCN(Cc2cccc(OCc3ccccc3)c2)C1] | 460.1 | 1.1 |
| Example 377 | 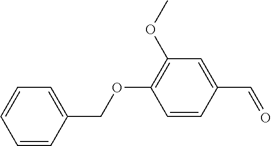 [SMILES: COc1cc(C=O)ccc1OCc1ccccc1] | 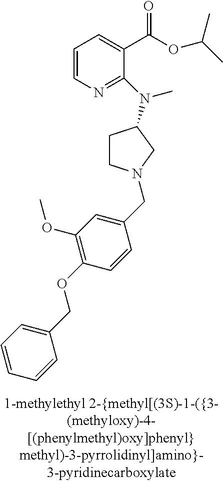 [SMILES: COc1cc(CN2CC[C@H](N(C)c3ncccc3C(=O)OC(C)C)C2)ccc1OCc1ccccc1] | 490.1 | 1.1 |
| Example 378 | 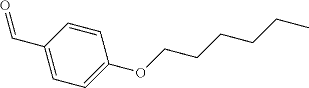 | 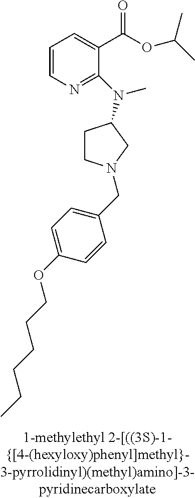 [SMILES: CCCCCCOc1ccc(CN2CC[C@H](N(C)c3ncccc3C(=O)OC(C)C)C2)cc1] | 454.17 | 1.2 |
| Example 379 | 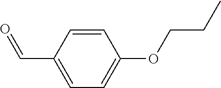 | 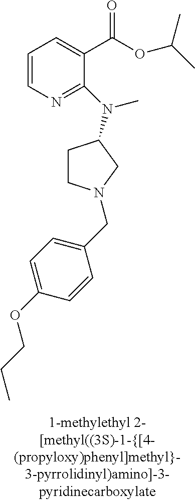 [SMILES: CCCOc1ccc(CN2CC[C@H](N(C)c3ncccc3C(=O)OC(C)C)C2)cc1] | 412.13 | 1.0 |
| Example 380 | 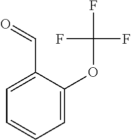 [SMILES: O=Cc1ccccc1OC(F)(F)F] | 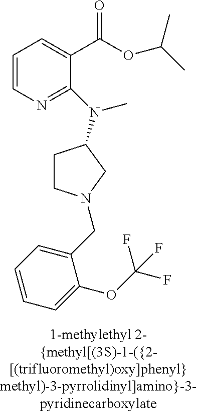 [SMILES: CC(C)OC(=O)c1cccnc1N(C)[C@H]1CCN(Cc2ccccc2OC(F)(F)F)C1] | 438.04 | 1.0 |
| Example 381 | 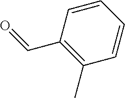 | 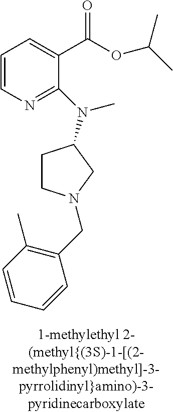 [SMILES: Cc1ccccc1CN1CC[C@H](N(C)c2ncccc2C(=O)OC(C)C)C1] | 368.13 | 1.0 |
| Example 382 | 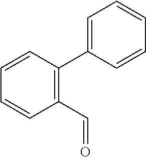 [SMILES: O=Cc1ccccc1-c1ccccc1] | 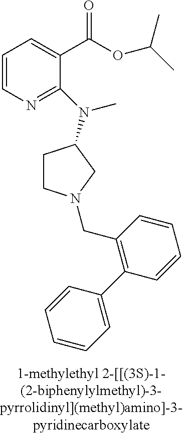 [SMILES: CC(C)OC(=O)c1cccnc1N(C)[C@H]1CCN(Cc2ccccc2-c2ccccc2)C1] | 430.08 | 1.1 |
| Example 383 | 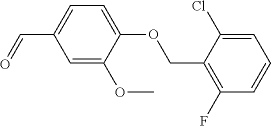 [SMILES: COc1cc(C=O)ccc1OCc1c(F)cccc1Cl] | 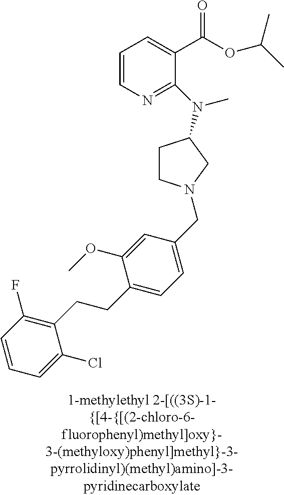 [SMILES: COc1cc(CN2CC[C@H](N(C)c3ncccc3C(=O)OC(C)C)C2)ccc1CCc1c(F)cccc1Cl] | 542 | 1.1 |
| Example 384 | 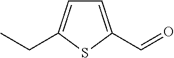 | 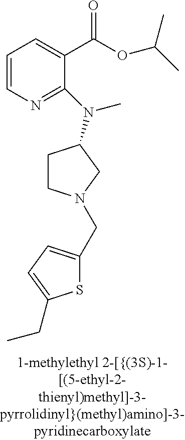 [SMILES: CCc1ccc(CN2CC[C@H](N(C)c3ncccc3C(=O)OC(C)C)C2)s1] | 388.07 | 1.0 |
| Example 385 | 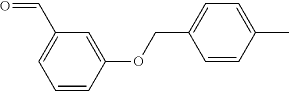 [SMILES: Cc1ccc(COc2cccc(C=O)c2)cc1] | 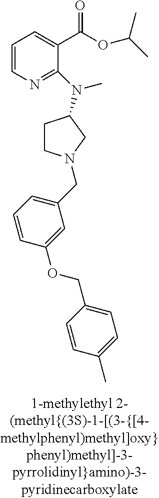 [SMILES: Cc1ccc(COc2cccc(CN3CC[C@H](N(C)c4ncccc4C(=O)OC(C)C)C3)c2)cc1] | 474.15 | 1.2 |
| Example 386 | 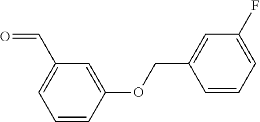 [SMILES: O=Cc1cccc(OCc2cccc(F)c2)c1] | 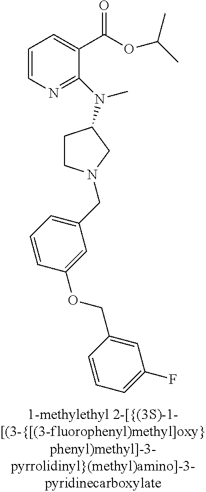 [SMILES: CC(C)OC(=O)c1cccnc1N(C)[C@H]1CCN(Cc2cccc(OCc3cccc(F)c3)c2)C1] | 478.1 | 1.1 |
| Example 387 | 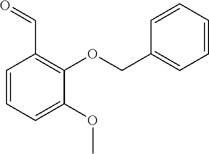 [SMILES: COc1cccc(C=O)c1OCc1ccccc1] | 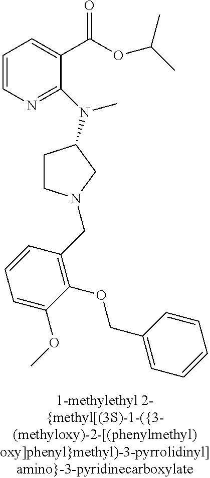 [SMILES: COc1cccc(CN2CC[C@H](N(C)c3ncccc3C(=O)OC(C)C)C2)c1OCc1ccccc1] | 476.1 | 1.1 |
| Example 388 | 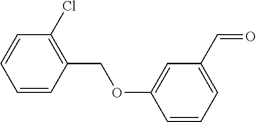 [SMILES: O=Cc1cccc(OCc2ccccc2Cl)c1] | 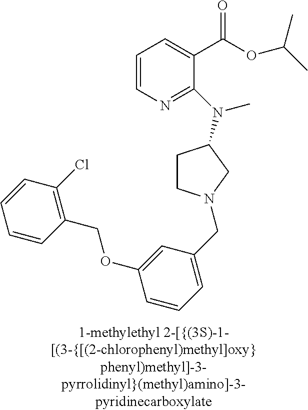 [SMILES: CC(C)OC(=O)c1cccnc1N(C)[C@H]1CCN(Cc2cccc(OCc3ccccc3Cl)c2)C1] | 494 | 1.2 |
| Example 389 | 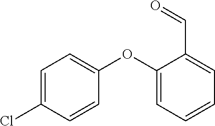 [SMILES: O=Cc1ccccc1Oc1ccc(Cl)cc1] | 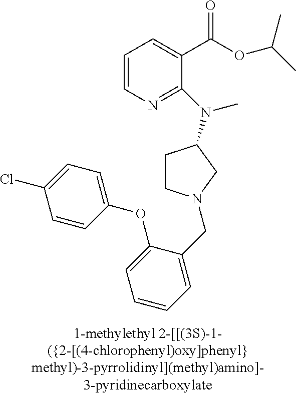 [SMILES: CC(C)OC(=O)c1cccnc1N(C)[C@H]1CCN(Cc2ccccc2Oc2ccc(Cl)cc2)C1] | 480 | 1.2 |
| Example 390 | 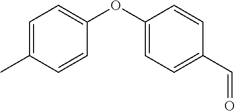 [SMILES: Cc1ccc(Oc2ccc(C=O)cc2)cc1] | 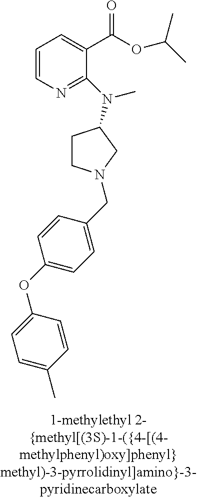 [SMILES: Cc1ccc(Oc2ccc(CN3CC[C@H](N(C)c4ncccc4C(=O)OC(C)C)C3)cc2)cc1] | 461.1 | 1.2 |
| Example 391 | 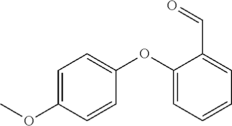 [SMILES: COc1ccc(Oc2ccccc2C=O)cc1] | 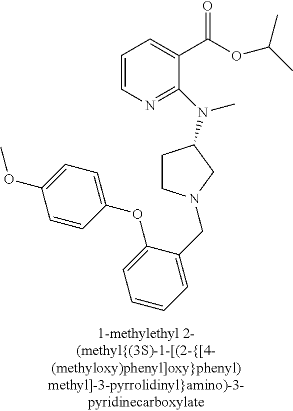 [SMILES: COc1ccc(Oc2ccccc2CN2CC[C@H](N(C)c3ncccc3C(=O)OC(C)C)C2)cc1] | 476 | 1.1 |
| Example 392 | 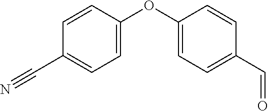 [SMILES: N#Cc1ccc(Oc2ccc(C=O)cc2)cc1] | 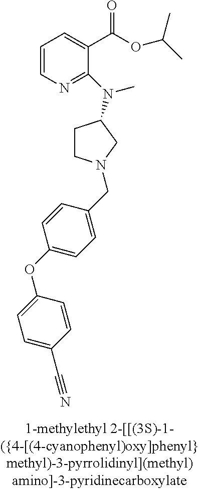 [SMILES: CC(C)OC(=O)c1cccnc1N(C)[C@H]1CCN(Cc2ccc(Oc3ccc(C#N)cc3)cc2)C1] | 471.1 | 1.0 |
| Example 393 | 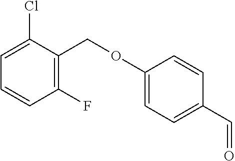 [SMILES: O=Cc1ccc(OCc2c(F)cccc2Cl)cc1] | 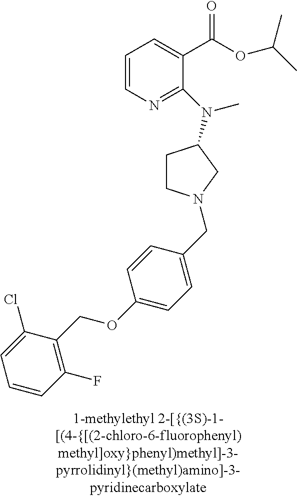 [SMILES: CC(C)OC(=O)c1cccnc1N(C)[C@H]1CCN(Cc2ccc(OCc3c(F)cccc3Cl)cc2)C1] | 512 | 1.1 |
| Example 394 | | 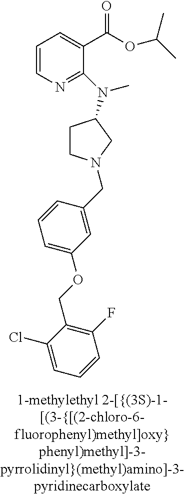 [SMILES: CC(C)OC(=O)c1cccnc1N(C)[C@H]1CCN(Cc2cccc(OCc3c(F)cccc3Cl)c2)C1] | 512 | 1.1 |

### Table 19

### Example 395

### 1-methylethyl 2-(4-{[3-({[3,4-bis(methyloxy)phenyl]methyl}oxy)phenyl]methyl}-1-piperazinyl)-3-pyridinecarboxylate

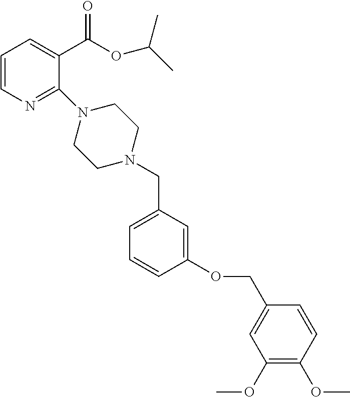 [SMILES: COc1ccc(COc2cccc(CN3CCN(c4ncccc4C(=O)OC(C)C)CC3)c2)cc1OC]

In a vial, 1-methylethyl 2-{4-[(3-hydroxyphenyl)methyl]-1-piperazinyl}-3-pyridinecarboxylate (30 mg, 0.084 mmol) and [3,4-bis(methyloxy)phenyl]methanol (0.127 mmol) were dissovled in DCM (1.5 ml) with Ph₃P (44.3 mg, 0.169 mmol). The solution was stirred for 15 min with ice-bath. Then DEAD (26.7 μl, 0.169 mmol) was added. The resulted solution was stirred at room temperature for 12 hours. The polymer was filtered and the resulting solution was purified by preparatory HPLC (basic condition) to afford 4.32 mg of the title compound. LC-MS m/z 506.3 (M+H)⁺, 0.96 min.

Following the procedure as described above in the preparation of 1-methylethyl 2-(4-{[3-({[3,4-bis(methyloxy)phenyl]methyl}oxy)phenyl]methyl}-1-piperazinyl)-3-pyridine carboxylate, 1-methylethyl 2-{4-[(3-hydroxyphenyl)methyl]-1-piperazinyl}-3-pyridine carboxylate (30 mg, 0.084 mmol) was reacted with the appropriate alcohol to yield the examples listed in Table XIX.

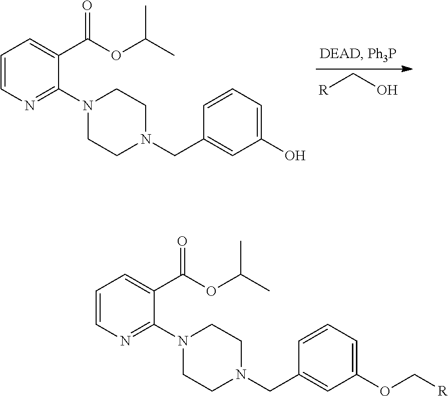

TABLE XIX

| | | | LC-MS | |
|---|---|---|---|---|
| | Aldehyde | | m/z | RT |
| Example | (ketone) | Product Name | (M + H)⁺ | (min) |
| Example 396 | 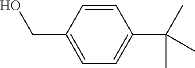 | 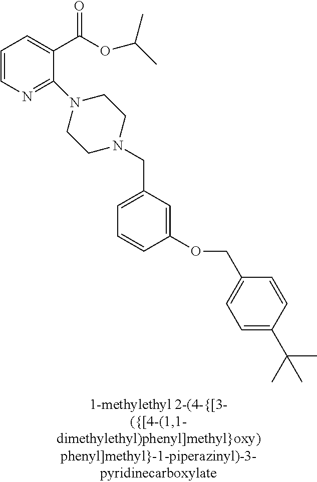 [SMILES: CC(C)OC(=O)c1cccnc1N1CCN(Cc2cccc(OCc3ccc(C(C)(C)C)cc3)c2)CC1] | 502.3 | 1.2 |
| Example 397 | 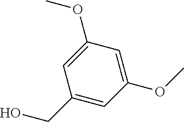 | 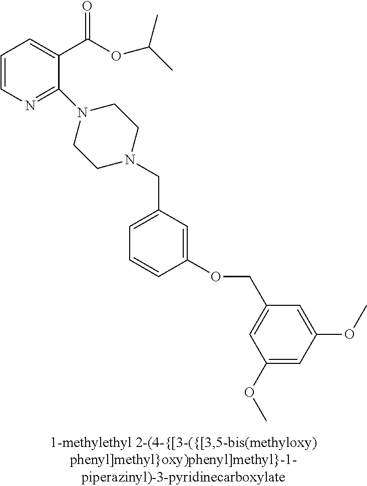 [SMILES: COc1cc(COc2cccc(CN3CCN(c4ncccc4C(=O)OC(C)C)CC3)c2)cc(OC)c1] | 506.3 | 1.0 |
| Example 398 | 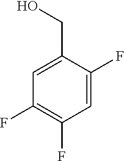 [SMILES: OCc1cc(F)c(F)cc1F] | 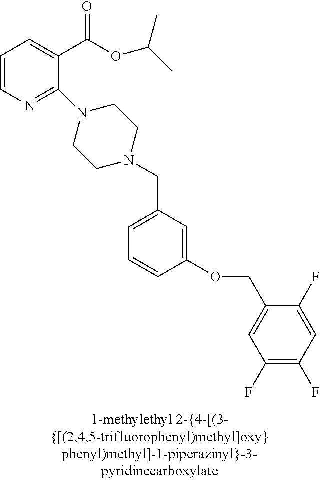 [SMILES: CC(C)OC(=O)c1cccnc1N1CCN(Cc2cccc(OCc3cc(F)c(F)cc3F)c2)CC1] | 500.4 | 1.1 |
| Example 399 | 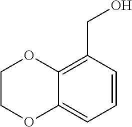 [SMILES: OCc1cccc2c1OCCO2] | 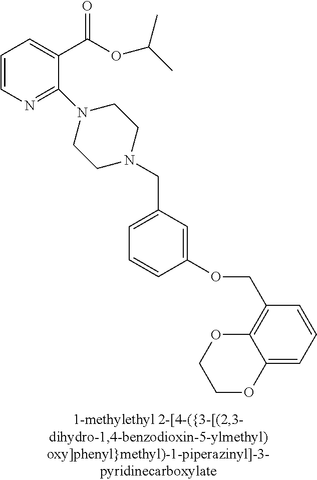 [SMILES: CC(C)OC(=O)c1cccnc1N1CCN(Cc2cccc(OCc3cccc4c3OCCO4)c2)CC1] | 504.2 | 1.0 |
| Example 400 | 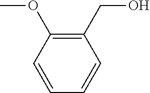 | 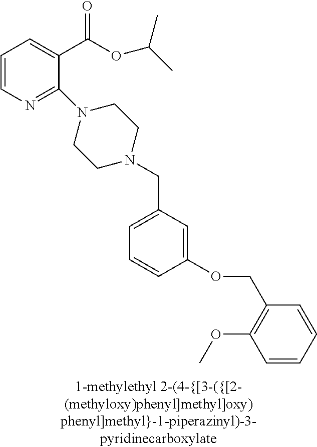 [SMILES: COc1ccccc1COc1cccc(CN2CCN(c3ncccc3C(=O)OC(C)C)CC2)c1] | 476.1 | 1.0 |
| Example 401 | 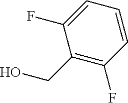 | 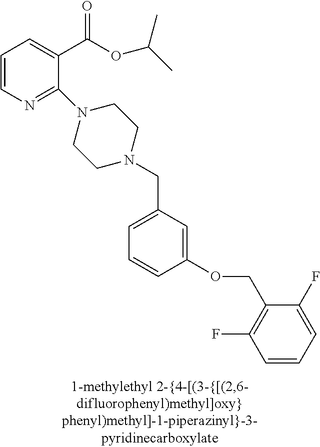 [SMILES: CC(C)OC(=O)c1cccnc1N1CCN(Cc2cccc(OCc3c(F)cccc3F)c2)CC1] | 481.9 | 1.0 |
| Example 402 | 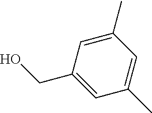 | 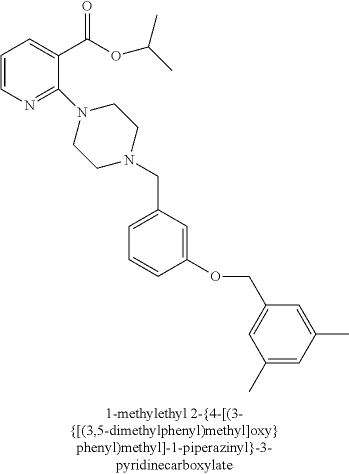 [SMILES: Cc1cc(C)cc(COc2cccc(CN3CCN(c4ncccc4C(=O)OC(C)C)CC3)c2)c1] | 474.3 | 1.1 |
| Example 403 | 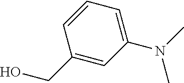 | 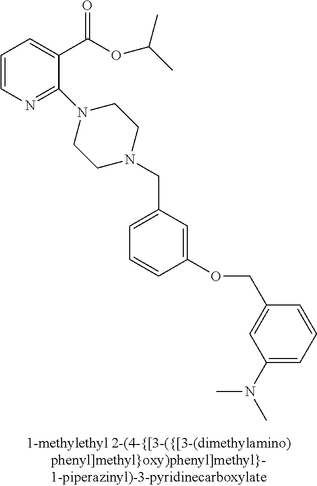 [SMILES: CC(C)OC(=O)c1cccnc1N1CCN(Cc2cccc(OCc3cccc(N(C)C)c3)c2)CC1] | 489.3 | 0.9 |
| Example 404 | 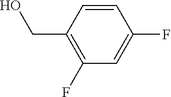 | 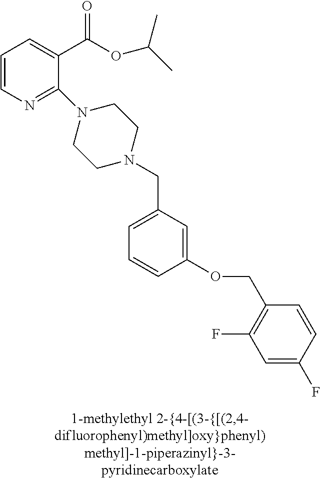 [SMILES: CC(C)OC(=O)c1cccnc1N1CCN(Cc2cccc(OCc3ccc(F)cc3F)c2)CC1] | 482.0 | 1.0 |
| Example 405 | 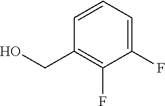 | 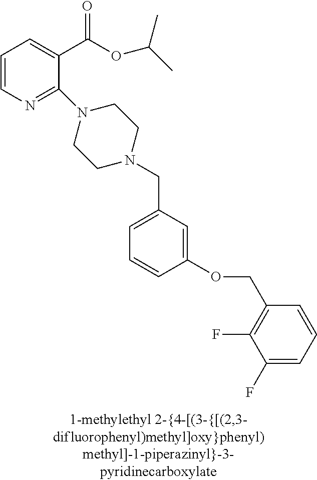 [SMILES: CC(C)OC(=O)c1cccnc1N1CCN(Cc2cccc(OCc3cccc(F)c3F)c2)CC1] | 482.0 | 1.0 |
| Example 406 | 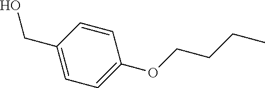 | 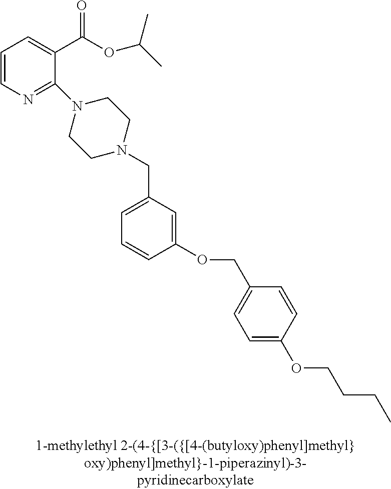 [SMILES: CCCCOc1ccc(COc2cccc(CN3CCN(c4ncccc4C(=O)OC(C)C)CC3)c2)cc1] | 518.4 | 1.2 |
| Example 407 | 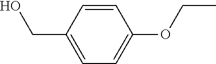 | 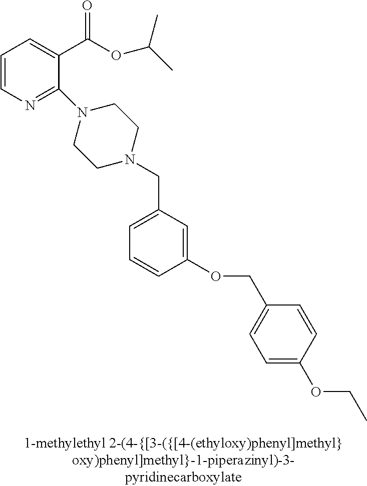 [SMILES: CCOc1ccc(COc2cccc(CN3CCN(c4ncccc4C(=O)OC(C)C)CC3)c2)cc1] | 490.2 | 1.1 |
| Example 408 | 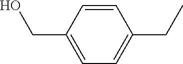 | 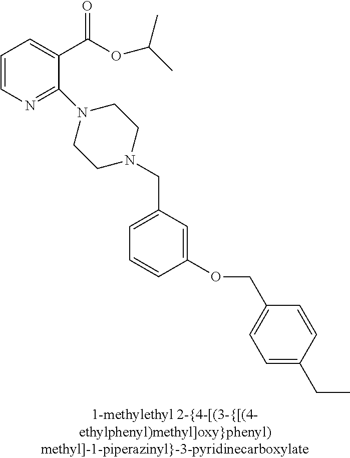 [SMILES: CCc1ccc(COc2cccc(CN3CCN(c4ncccc4C(=O)OC(C)C)CC3)c2)cc1] | 474.4 | 1.1 |
| Example 409 | 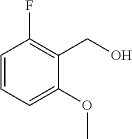 | 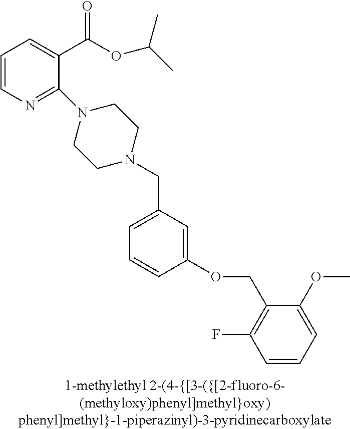 [SMILES: COc1cccc(F)c1COc1cccc(CN2CCN(c3ncccc3C(=O)OC(C)C)CC2)c1] | 494.2 | 1.0 |
| Example 410 | 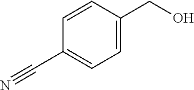 | 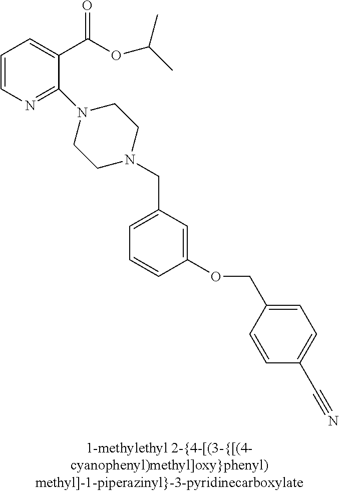 [SMILES: CC(C)OC(=O)c1cccnc1N1CCN(Cc2cccc(OCc3ccc(C#N)cc3)c2)CC1] | 471.3 | 1.0 |
| Example 411 | 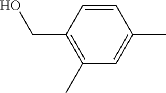 | 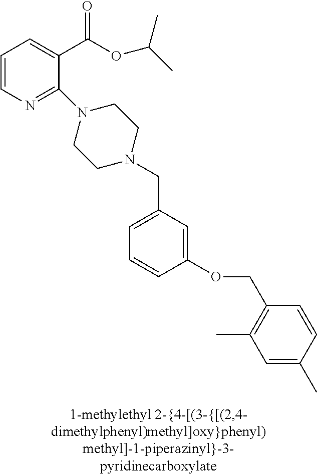 [SMILES: Cc1ccc(COc2cccc(CN3CCN(c4ncccc4C(=O)OC(C)C)CC3)c2)c(C)c1] | 474.3 | 1.1 |
| Example 412 | 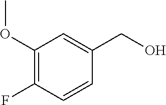 | 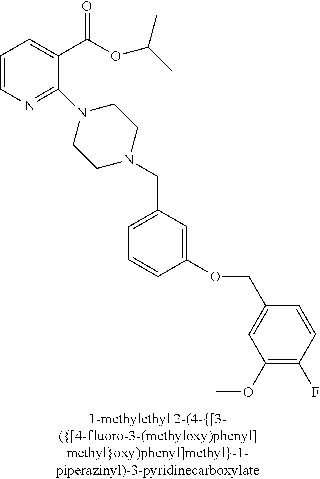 [SMILES: COc1cc(COc2cccc(CN3CCN(c4ncccc4C(=O)OC(C)C)CC3)c2)ccc1F] | 494.1 | 1.0 |
| Example 413 | 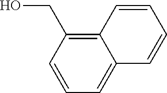 [SMILES: OCc1cccc2ccccc12] | 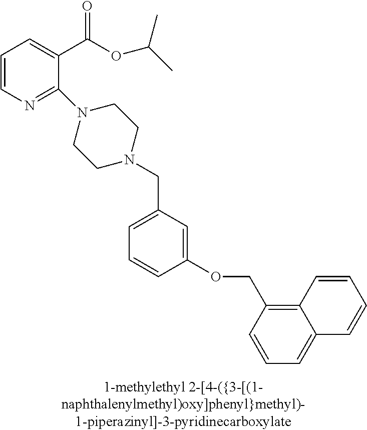 [SMILES: CC(C)OC(=O)c1cccnc1N1CCN(Cc2cccc(OCc3cccc4ccccc34)c2)CC1] | 496.3 | 1.1 |
| Example 414 | 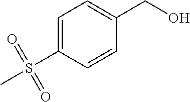 | 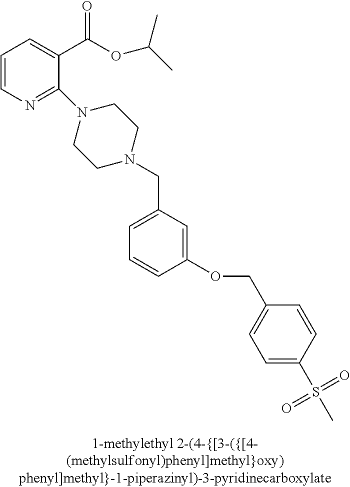 [SMILES: CC(C)OC(=O)c1cccnc1N1CCN(Cc2cccc(OCc3ccc(S(C)(=O)=O)cc3)c2)CC1] | 524.3 | 0.9 |
| Example 415 | 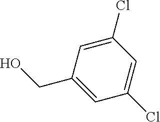 | 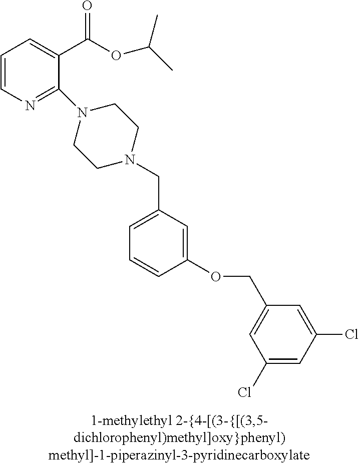 [SMILES: CC(C)OC(=O)c1cccnc1N1CCN(Cc2cccc(OCc3cc(Cl)cc(Cl)c3)c2)CC1] | 514.2 | 1.1 |
| Example 416 | 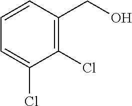 | 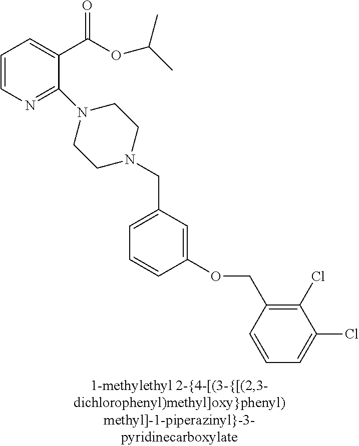 [SMILES: CC(C)OC(=O)c1cccnc1N1CCN(Cc2cccc(OCc3cccc(Cl)c3Cl)c2)CC1] | 514.2 | 1.1 |
| Example 417 | 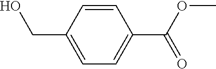 | 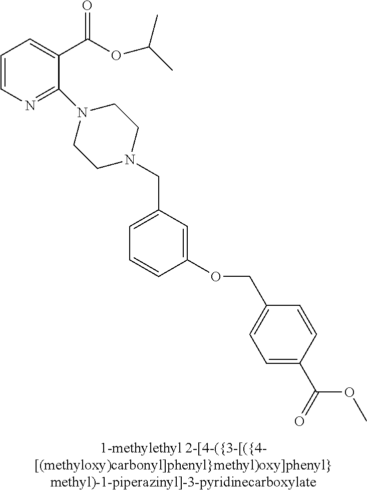 [SMILES: COC(=O)c1ccc(COc2cccc(CN3CCN(c4ncccc4C(=O)OC(C)C)CC3)c2)cc1] | 504.1 | 1.0 |
| Example 418 | 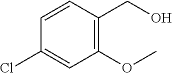 | 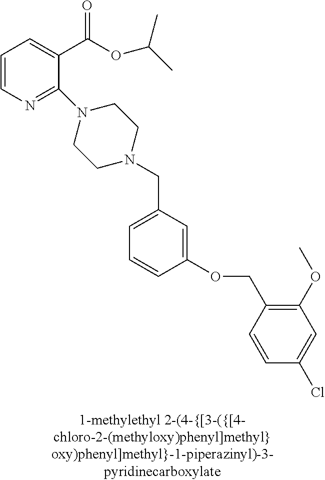 [SMILES: COc1cc(Cl)ccc1COc1cccc(CN2CCN(c3ncccc3C(=O)OC(C)C)CC2)c1] | 510.1 | 1.1 |
| Example 419 | 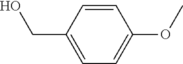 | 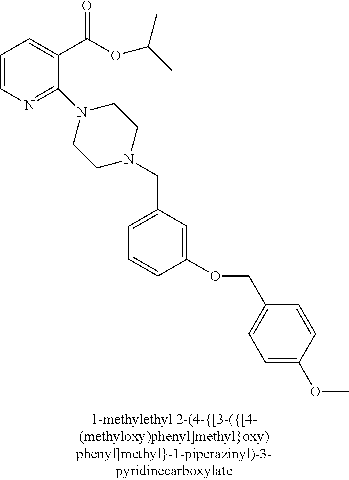 [SMILES: COc1ccc(COc2cccc(CN3CCN(c4ncccc4C(=O)OC(C)C)CC3)c2)cc1] | 476.1 | 1.0 |
| Example 420 | 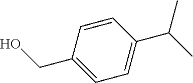 | 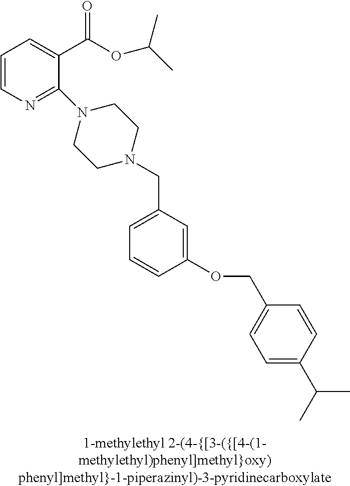 [SMILES: CC(C)OC(=O)c1cccnc1N1CCN(Cc2cccc(OCc3ccc(C(C)C)cc3)c2)CC1] | 488.2 | 1.2 |
| Example 421 | 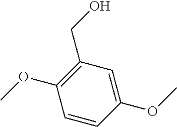 | 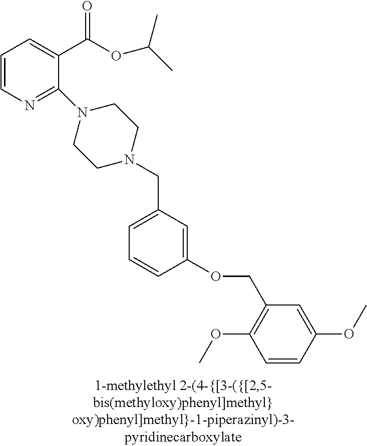 [SMILES: COc1ccc(OC)c(COc2cccc(CN3CCN(c4ncccc4C(=O)OC(C)C)CC3)c2)c1] | 506.3 | 1.0 |
| Example 422 | 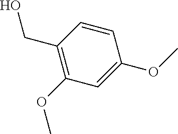 | 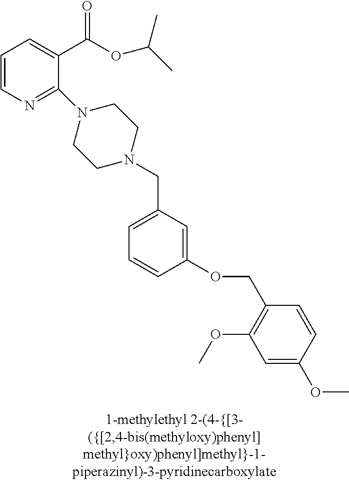 [SMILES: COc1ccc(COc2cccc(CN3CCN(c4ncccc4C(=O)OC(C)C)CC3)c2)c(OC)c1] | 506.2 | 1.0 |

### Table 20

Following the procedure as described above in the preparation of 1-methylethyl 2-(4-{[3-({[3,4-bis(methyloxy)phenyl]methyl}oxy)phenyl]methyl}-1-piperazinyl)-3-pyridine carboxylate, 1-methylethyl 2-{4[(4-hydroxyphenyl)methyl]-1-piperazinyl}-3-pyridine carboxylate (30 mg, 0.084 mmol) was reacted with the appropriate alcohol to yield the examples listed in Table XX.

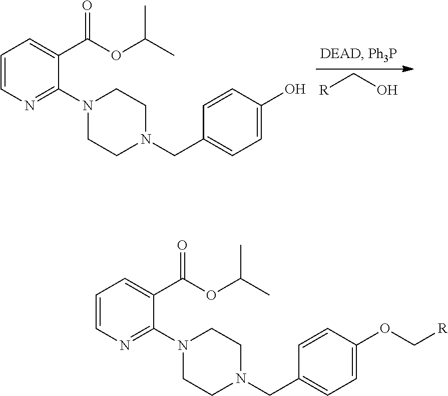

TABLE XX

| | | | LC-MS | |
|---|---|---|---|---|
| | Aldehyde | | m/z | RT |
| Example | (ketone) | Product Name | (M + H)⁺ | (min) |
| Example 423 | 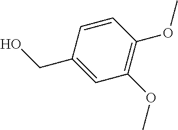 | 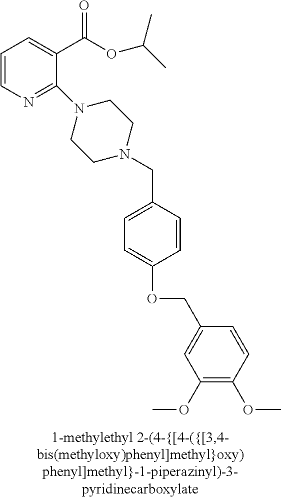 [SMILES: COc1ccc(COc2ccc(CN3CCN(c4ncccc4C(=O)OC(C)C)CC3)cc2)cc1OC] | 506 | 1.2 |
| Example 424 | 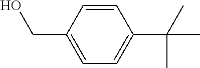 | 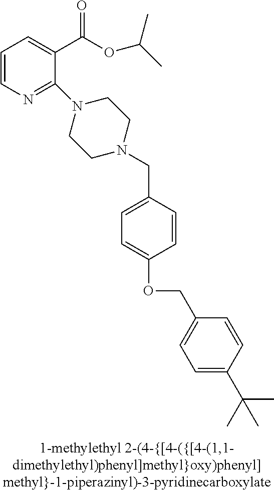 [SMILES: CC(C)OC(=O)c1cccnc1N1CCN(Cc2ccc(OCc3ccc(C(C)(C)C)cc3)cc2)CC1] | 502.1 | 1.3 |
| Example 425 | 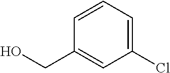 | 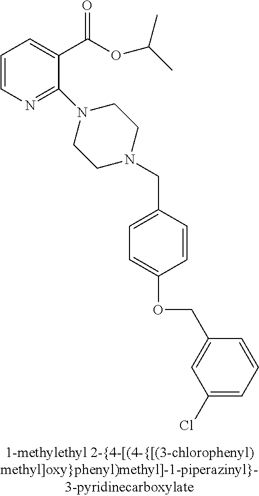 [SMILES: CC(C)OC(=O)c1cccnc1N1CCN(Cc2ccc(OCc3cccc(Cl)c3)cc2)CC1] | 480 | 1.0 |
| Example 426 | 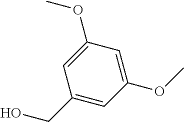 | 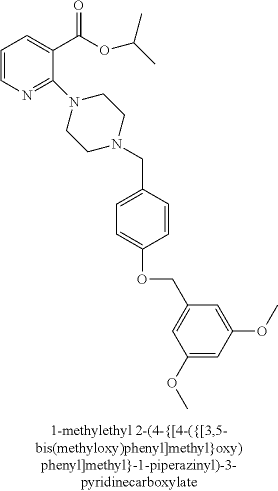 [SMILES: COc1cc(COc2ccc(CN3CCN(c4ncccc4C(=O)OC(C)C)CC3)cc2)cc(OC)c1] | 506 | 1.1 |
| Example 427 | 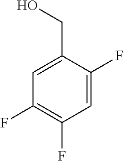 [SMILES: OCc1cc(F)c(F)cc1F] | 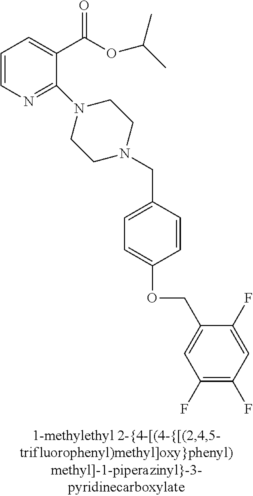 [SMILES: CC(C)OC(=O)c1cccnc1N1CCN(Cc2ccc(OCc3cc(F)c(F)cc3F)cc2)CC1] | 500 | 1.1 |
| Example 428 | 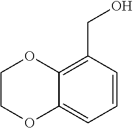 [SMILES: OCc1cccc2c1OCCO2] | 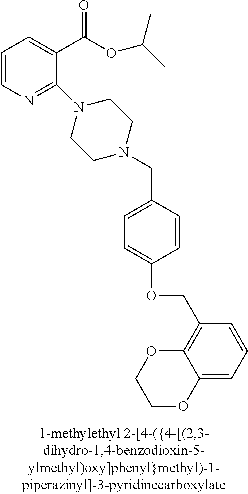 [SMILES: CC(C)OC(=O)c1cccnc1N1CCN(Cc2ccc(OCc3cccc4c3OCCO4)cc2)CC1] | 504.06 | 1.0 |
| Example 429 | 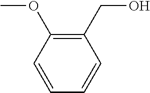 | 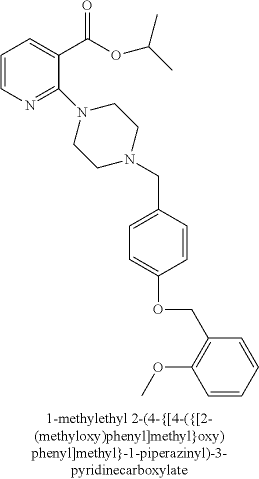 [SMILES: COc1ccccc1COc1ccc(CN2CCN(c3ncccc3C(=O)OC(C)C)CC2)cc1] | 476 | 1.1 |
| Example 430 | 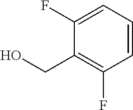 | 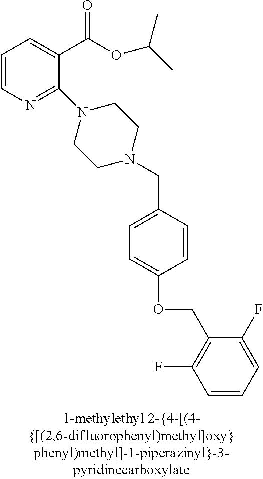 [SMILES: CC(C)OC(=O)c1cccnc1N1CCN(Cc2ccc(OCc3c(F)cccc3F)cc2)CC1] | 482 | 1.1 |
| Example 431 | 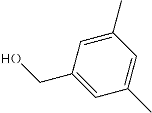 | 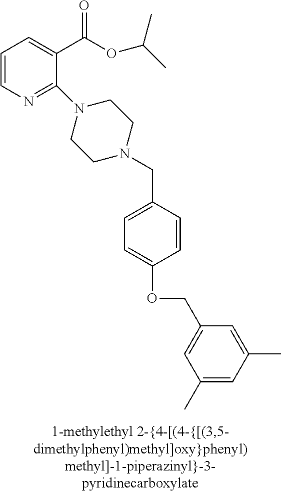 [SMILES: Cc1cc(C)cc(COc2ccc(CN3CCN(c4ncccc4C(=O)OC(C)C)CC3)cc2)c1] | 474 | 1.2 |
| Example 432 | 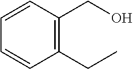 | 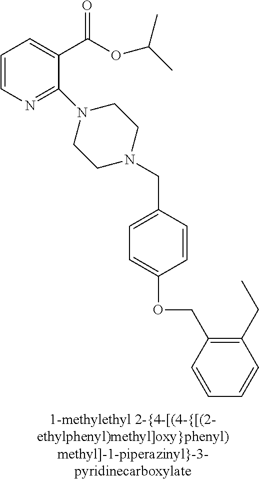 [SMILES: CCc1ccccc1COc1ccc(CN2CCN(c3ncccc3C(=O)OC(C)C)CC2)cc1] | 474 | 1.1 |
| Example 433 | 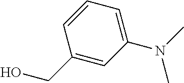 | 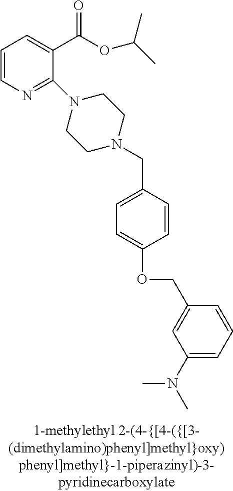 [SMILES: CC(C)OC(=O)c1cccnc1N1CCN(Cc2ccc(OCc3cccc(N(C)C)c3)cc2)CC1] | 1 | 0.9 |
| Example 434 | 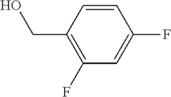 | 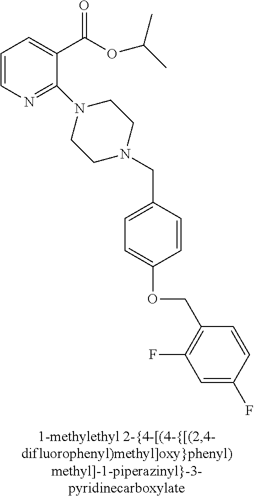 [SMILES: CC(C)OC(=O)c1cccnc1N1CCN(Cc2ccc(OCc3ccc(F)cc3F)cc2)CC1] | 489 | 1.1 |
| Example 435 | 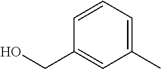 | 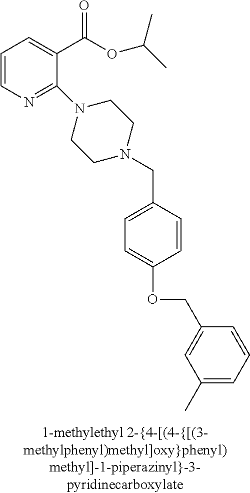 [SMILES: Cc1cccc(COc2ccc(CN3CCN(c4ncccc4C(=O)OC(C)C)CC3)cc2)c1] | 460.1 | 1.1 |
| Example 436 | 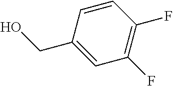 | 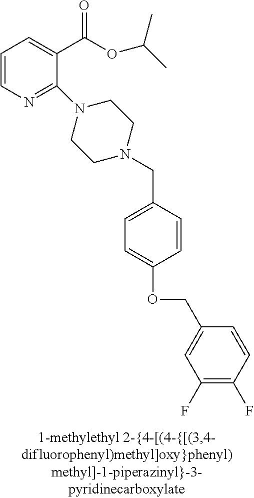 [SMILES: CC(C)OC(=O)c1cccnc1N1CCN(Cc2ccc(OCc3ccc(F)c(F)c3)cc2)CC1] | 482 | 1.1 |
| Example 437 | 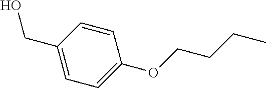 | 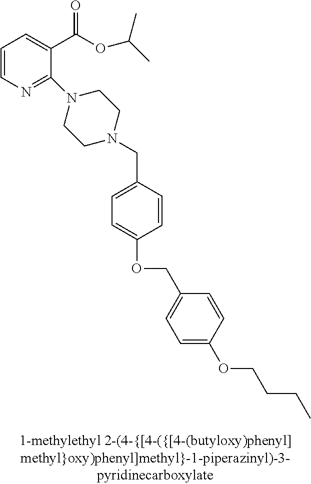 [SMILES: CCCCOc1ccc(COc2ccc(CN3CCN(c4ncccc4C(=O)OC(C)C)CC3)cc2)cc1] | 518 | 1.2 |
| Example 438 | 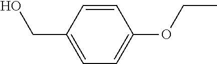 | 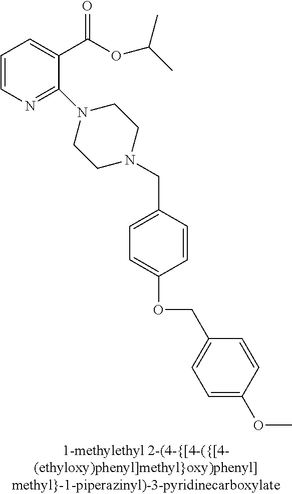 [SMILES: COc1ccc(COc2ccc(CN3CCN(c4ncccc4C(=O)OC(C)C)CC3)cc2)cc1] | 490 | 1.1 |
| Example 439 | 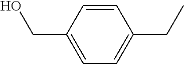 | 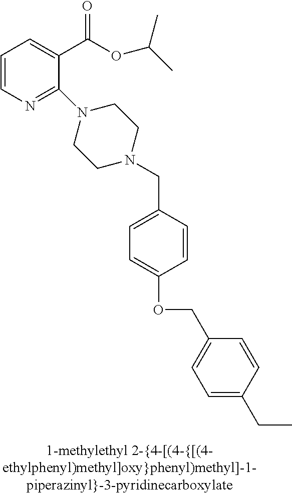 [SMILES: CCc1ccc(COc2ccc(CN3CCN(c4ncccc4C(=O)OC(C)C)CC3)cc2)cc1] | 474 | 1.2 |
| Example 440 | 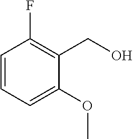 | 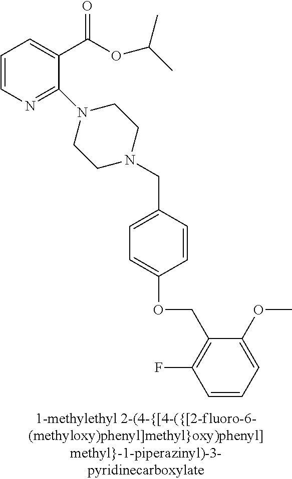 [SMILES: COc1cccc(F)c1COc1ccc(CN2CCN(c3ncccc3C(=O)OC(C)C)CC2)cc1] | 494 | 1.1 |
| Example 441 | 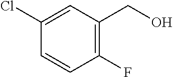 | 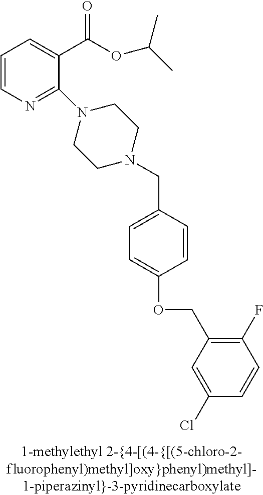 [SMILES: CC(C)OC(=O)c1cccnc1N1CCN(Cc2ccc(OCc3cc(Cl)ccc3F)cc2)CC1] | 498 | 1.1 |
| Example 442 | 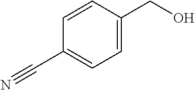 | 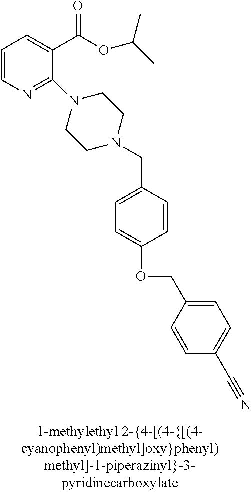 [SMILES: CC(C)OC(=O)c1cccnc1N1CCN(Cc2ccc(OCc3ccc(C#N)cc3)cc2)CC1] | 471 | 1.0 |
| Example 443 | 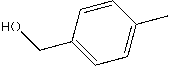 | 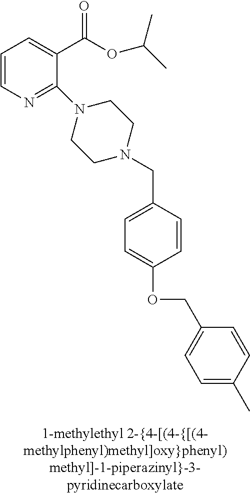 [SMILES: Cc1ccc(COc2ccc(CN3CCN(c4ncccc4C(=O)OC(C)C)CC3)cc2)cc1] | 460.1 | 1.1 |
| Example 444 | 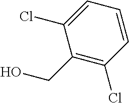 | 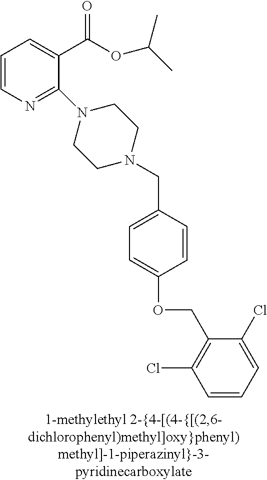 [SMILES: CC(C)OC(=O)c1cccnc1N1CCN(Cc2ccc(OCc3c(Cl)cccc3Cl)cc2)CC1] | 513.9 | 1.1 |
| Example 445 | 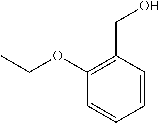 | 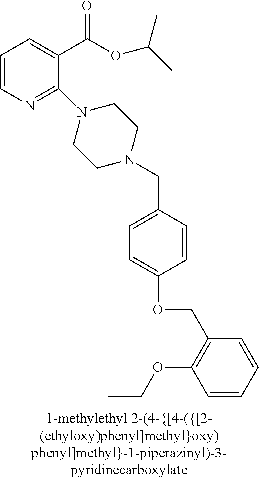 [SMILES: CCOc1ccccc1COc1ccc(CN2CCN(c3ncccc3C(=O)OC(C)C)CC2)cc1] | 490 | 1.1 |
| Example 446 | 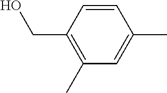 | 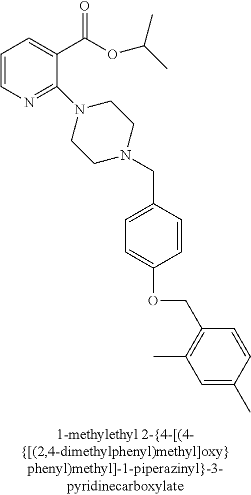 [SMILES: Cc1ccc(COc2ccc(CN3CCN(c4ncccc4C(=O)OC(C)C)CC3)cc2)c(C)c1] | 474 | 1.2 |
| Example 447 | 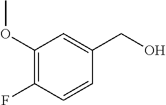 | 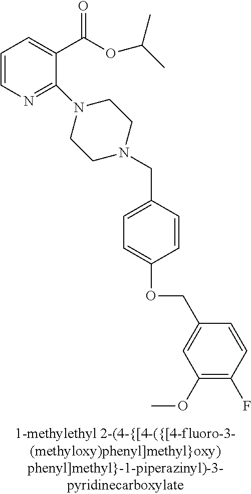 [SMILES: COc1cc(COc2ccc(CN3CCN(c4ncccc4C(=O)OC(C)C)CC3)cc2)ccc1F] | 494 | 1.1 |
| Example 448 | 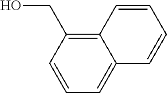 [SMILES: OCc1cccc2ccccc12] | 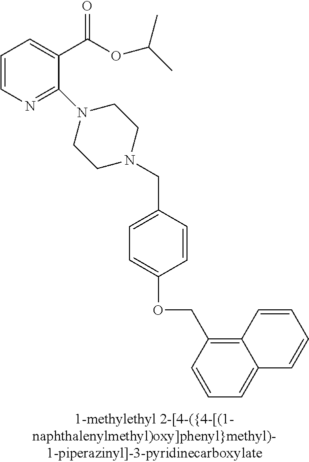 [SMILES: CC(C)OC(=O)c1cccnc1N1CCN(Cc2ccc(OCc3cccc4ccccc34)cc2)CC1] | 496 | 1.1 |
| Example 449 | 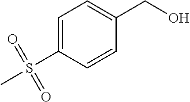 | 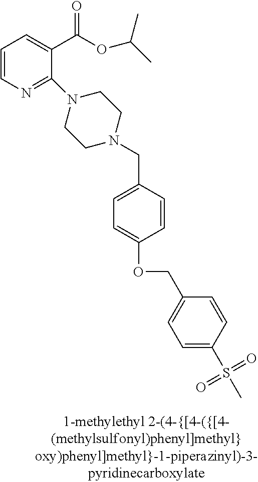 [SMILES: CC(C)OC(=O)c1cccnc1N1CCN(Cc2ccc(OCc3ccc(S(C)(=O)=O)cc3)cc2)CC1] | 524.04 | 0.9 |
| Example 450 | 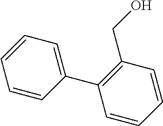 [SMILES: OCc1ccccc1-c1ccccc1] | 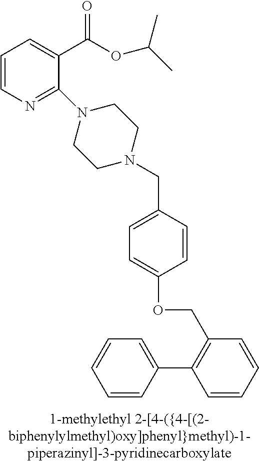 [SMILES: CC(C)OC(=O)c1cccnc1N1CCN(Cc2ccc(OCc3ccccc3-c3ccccc3)cc2)CC1] | 522 | 1.2 |
| Example 451 | 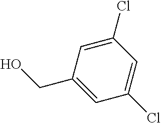 | 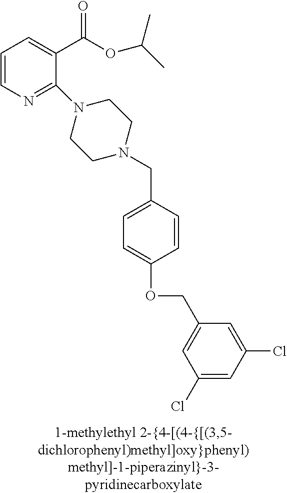 [SMILES: CC(C)OC(=O)c1cccnc1N1CCN(Cc2ccc(OCc3cc(Cl)cc(Cl)c3)cc2)CC1] | 513.9 | 1.2 |
| Example 452 | 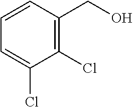 | 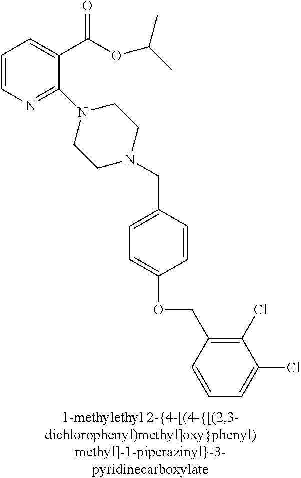 [SMILES: CC(C)OC(=O)c1cccnc1N1CCN(Cc2ccc(OCc3cccc(Cl)c3Cl)cc2)CC1] | 522 | 1.2 |
| Example 453 | 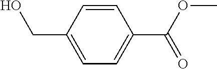 | 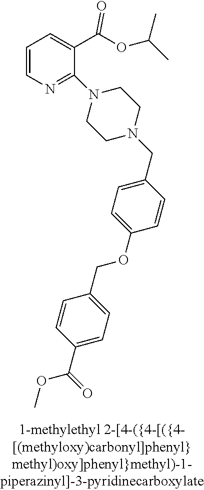 [SMILES: COC(=O)c1ccc(COc2ccc(CN3CCN(c4ncccc4C(=O)OC(C)C)CC3)cc2)cc1] | 504.06 | 1.0 |
| Example 454 | 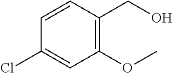 | 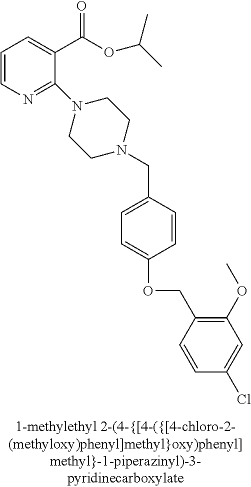 [SMILES: COc1cc(Cl)ccc1COc1ccc(CN2CCN(c3ncccc3C(=O)OC(C)C)CC2)cc1] | 510 | 1.1 |
| Example 455 | 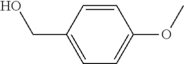 | 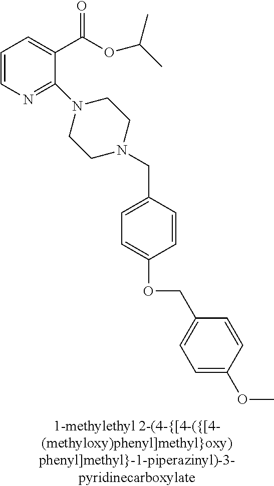 [SMILES: COc1ccc(COc2ccc(CN3CCN(c4ncccc4C(=O)OC(C)C)CC3)cc2)cc1] | 476.06 | 1.0 |
| Example 456 | 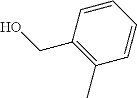 | 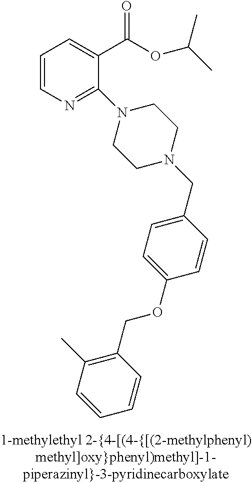 [SMILES: Cc1ccccc1COc1ccc(CN2CCN(c3ncccc3C(=O)OC(C)C)CC2)cc1] | 460.09 | 1.1 |
| Example 457 | 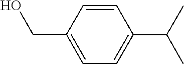 | 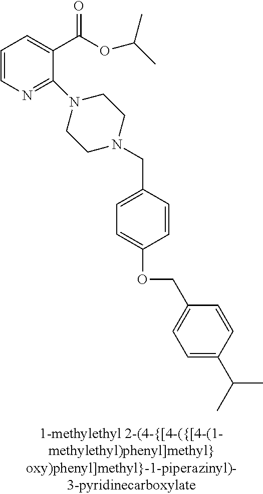 [SMILES: CC(C)OC(=O)c1cccnc1N1CCN(Cc2ccc(OCc3ccc(C(C)C)cc3)cc2)CC1] | 488 | 1.2 |
| Example 458 | 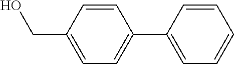 [SMILES: OCc1ccc(-c2ccccc2)cc1] | 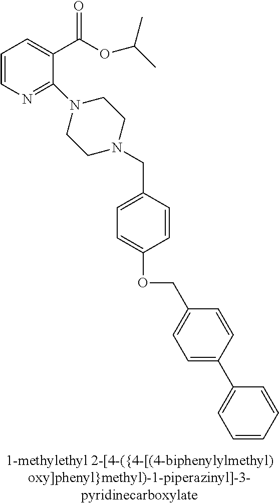 [SMILES: CC(C)OC(=O)c1cccnc1N1CCN(Cc2ccc(OCc3ccc(-c4ccccc4)cc3)cc2)CC1] | 522 | 1.2 |
| Example 459 | 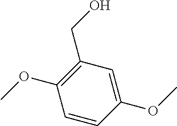 | 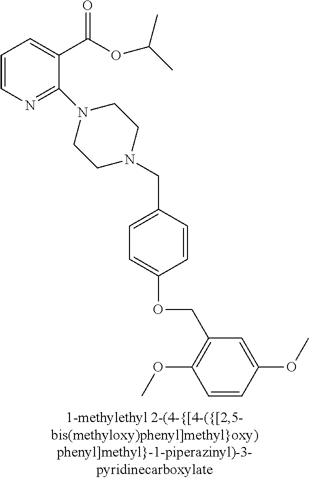 [SMILES: COc1ccc(OC)c(COc2ccc(CN3CCN(c4ncccc4C(=O)OC(C)C)CC3)cc2)c1] | 506 | 1.0 |
| Example 460 | 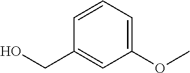 | 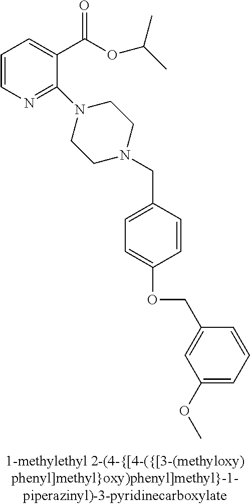 [SMILES: COc1cccc(COc2ccc(CN3CCN(c4ncccc4C(=O)OC(C)C)CC3)cc2)c1] | 476.06 | 1.0 |
| Example 461 | 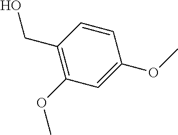 | 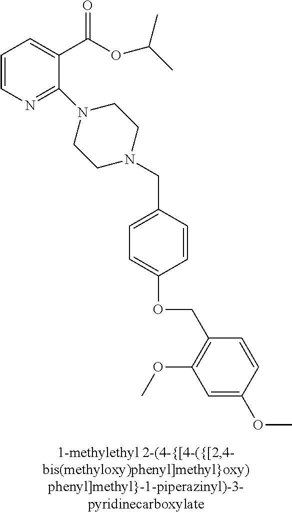 [SMILES: COc1ccc(COc2ccc(CN3CCN(c4ncccc4C(=O)OC(C)C)CC3)cc2)c(OC)c1] | 506 | 1 |
| Example 462 | 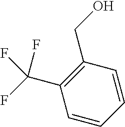 [SMILES: OCc1ccccc1C(F)(F)F] | 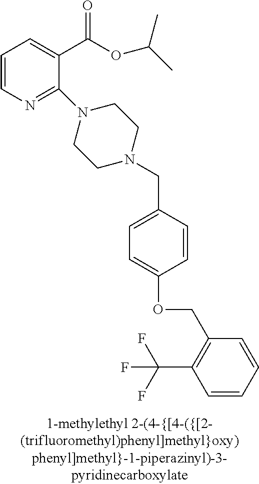 [SMILES: CC(C)OC(=O)c1cccnc1N1CCN(Cc2ccc(OCc3ccccc3C(F)(F)F)cc2)CC1] | 514.04 | 1.1 |

### Table 21

### Example 463

### 1-methylethyl 2-(4-{[4-({4-[(2-chloro-6-fluorophenyl)methyl]-1-piperazinyl}methyl)phenyl]methyl}-1-piperazinyl)-3-pyridinecarboxylate

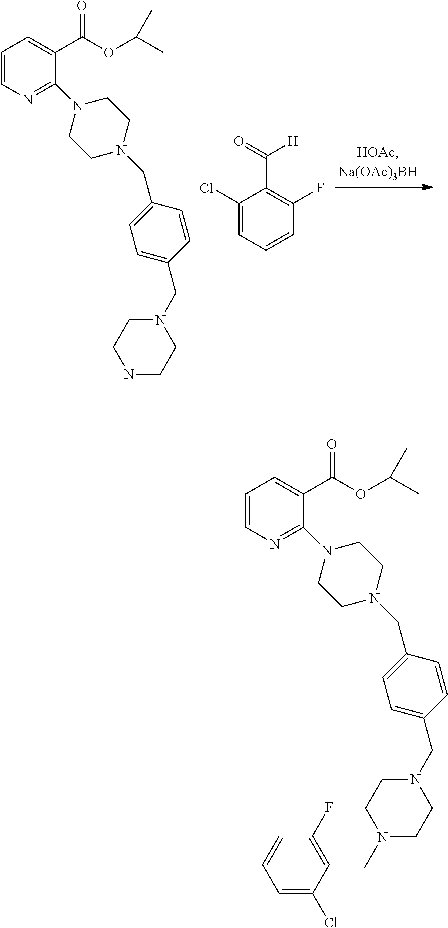

To a vial with 1-methylethyl 2-(4-{[4-(1-piperazinylmethyl)phenyl]methyl}-1-piperazinyl)-3-pyridinecarboxylate (130 mg, 0.297 mmol) in Dichloromethane (DCM) (5 mL) with 2-chloro-6-fluorobenzaldehyde (56.5 mg, 0.357 mmol) was added HOAc (17.84 mg, 0.297 mmol). The result solution was stirred for 2 hr. Na(OAc)₃BH (127 mg, 0.594 mmol) was added into the solution and stirred for another 12 hr. H₂O (10 mL) and DCM (10 mL) were added and the result solution was separated by Phase Separator. The water layer was washed with DCM (10 mL). Combined the organic layer and removed the solvent. The product was purified by prepared HPLC (Gilson, basic) to afford 88 mg (46.0%) of the desired product. LC/MS: m/z=580.3[M+H]⁺, Ret. Time: 0.74 min.

Following the procedure as described above in the preparation of 1-methylethyl 2-(4-{[4-({4-[(2-chloro-6-fluorophenyl)methyl]-1-piperazinyl}methyl)phenyl]methyl}-1-piperazinyl)-3-pyridinecarboxylate, 1-methylethyl 2-(4-{[4-(1-piperazinylmethyl)phenyl]methyl}-1-piperazinyl)-3-pyridinecarboxylate (40.0 mg, 0.091 mmol) was reacted with the appropriate benzyl aldehyde (0.137 mmol) to yield the examples listed in Table 1.

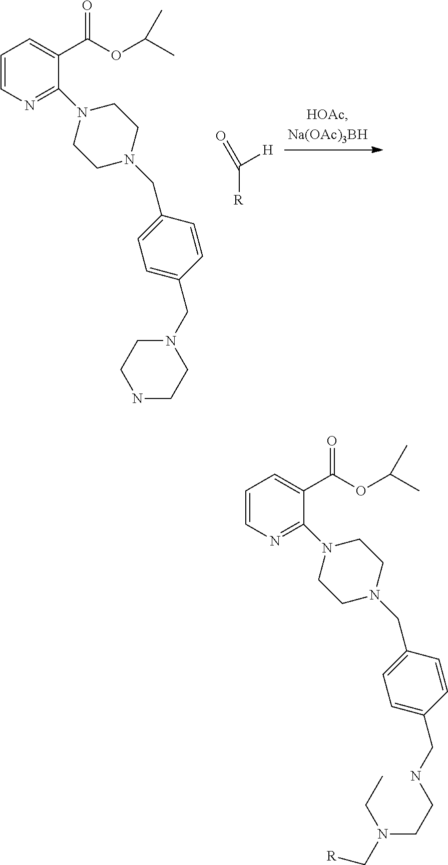

TABLE XXI

| | | | LC-MS | |
|---|---|---|---|---|
| | | | m/z | RT |
| Example | Aldehyde | Product | (M + H)⁺ | (min) |
| Example 464 | 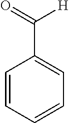 | 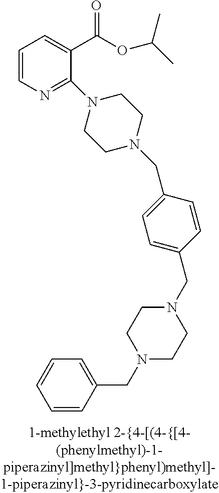 [SMILES: CC(C)OC(=O)c1cccnc1N1CCN(Cc2ccc(CN3CCN(Cc4ccccc4)CC3)cc2)CC1] | 525.8 | 0.65 |
| Example 465 | 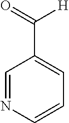 | 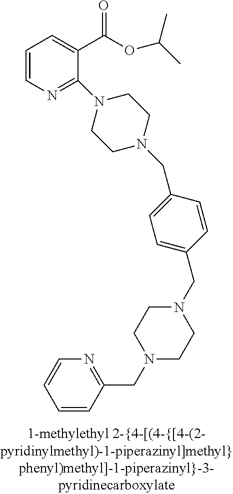 [SMILES: CC(C)OC(=O)c1cccnc1N1CCN(Cc2ccc(CN3CCN(Cc4ccccn4)CC3)cc2)CC1] | 529.5 | 0.55 |
| Example 466 | 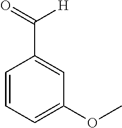 | 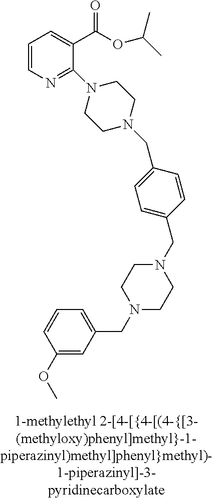 [SMILES: COc1cccc(CN2CCN(Cc3ccc(CN4CCN(c5ncccc5C(=O)OC(C)C)CC4)cc3)CC2)c1] | 558.5 | 0.70 |
| Example 467 | 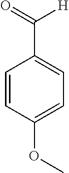 | 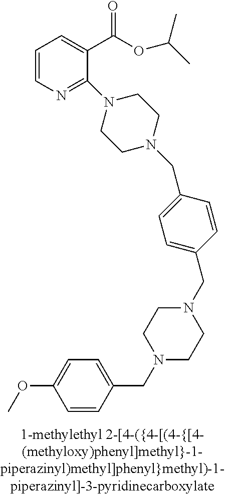 [SMILES: COc1ccc(CN2CCN(Cc3ccc(CN4CCN(c5ncccc5C(=O)OC(C)C)CC4)cc3)CC2)cc1] | 558.5 | 0.67 |

### Example 468

### 1-Methylethyl-2-{4-[(3-{[4-(methyloxy)phenyl]oxy}phenyl)methyl]-1-piperazinyl}-3-pyridine carboxylate dihydrochloride

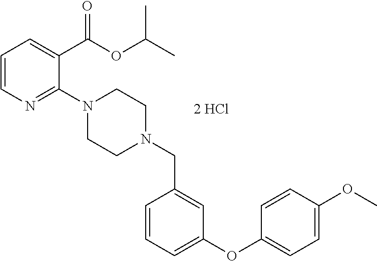 [SMILES: COc1ccc(Oc2cccc(CN3CCN(c4ncccc4C(=O)OC(C)C)CC3)c2)cc1]

The free base of 1-methylethyl 2-(1-piperazinyl)-3-pyridinecarboxylate (270 mg, 1.083 mmol), 3-{[4-(methyloxy)phenyl]oxy}benzaldehyde (494 mg, 2.166 mmol) and THF (5 mL) were combined in a 20 mL vial and stirred together for 5 min and then sodium triacetoxyborohydride (689 mg, 3.25 mmol) was added. The resulting mixture was capped and stirred 16 h and then diluted with EtOAc (75 mL) and washed in succession with 1 N aq NaOH, H₂O, and saturated aq NaCl, (25 mL each) and then the EtOAc phase was dried (Na₂SO₄) and concentrated to afford a brown oil. Purification by preparative hplc (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 217 mg (43%) of the free base of the title compound as a clear yellow oil. Lcms rt 0.92[M+H]=462.0

The above free base (217 mg, 0.436 mmol) was dissolved in diethyl ether (2 mL) and a solution of 2M HCl in diethyl ether (0.228 mL, 0.456 mmol) was added. The precipitated white solid was stirred in a sealed vial for 30 min and then was filtered and the resulting white solid was washed with more diethyl ether and dried in vacuo to afford the hydrochloride salt of the title compound (128.4 mg, 0.255 mmol, 24%) as a white solid. Lcms rt 0.98[M+H]=462.3

### Example 469

### 1-Methylethyl-2-(4-{[2′-(trifluoromethyl)-3-biphenylyl]methyl}-1-piperazinyl)-3-pyridine carboxylate

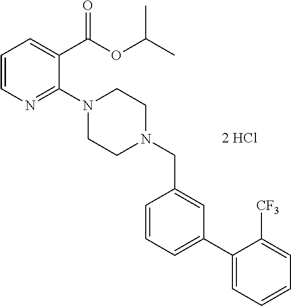 [SMILES: CC(C)OC(=O)c1cccnc1N1CCN(Cc2cccc(-c3ccccc3C(F)(F)F)c2)CC1]

1-Methylethyl 2-(1-piperazinyl)-3-pyridinecarboxylate (258 mg, 1.035 mmol) free base, 2′-(trifluoromethyl)-3-biphenylcarbaldehyde (518 mg, 2.070 mmol), and tetrahydrofuran (THF) (5.1 mL) were combined in a 20 mL vial and stirred together for 5 min and then sodium triacetoxyborohydride (658 mg, 3.10 mmol) was added. The mixture was stirred 6 h at 23° C. The reaction was diluted with EtOAc (75 mL) and washed with 1 N aq NaOH (25 mL), H₂O (25 mL) and satd aq NaCl (25 mL), dried (Na₂SO₄) and concentrated to afford a brown oil which was purified on a silica cartridge (12 g) eluting at 30 mL/min with a gradient running from 100% dichloromethane to 60% EtOAc/dichloromethane over 35 min. The desired fractions were pooled and concentrated to afford a brown oil which was further purified by preparative hplc. (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 146 mg (29%) of the title compound as a clear oil.

The above free base (146 mg, 0.302 mmol) was dissolved in diethyl ether (2 mL) and a solution of 2M HCl in diethyl ether (0.151 mL, 0.302 mmol) was added. The precipitated white solid was stirred in a sealed vial for 30 min and then was filtered and the resulting white solid was washed with more diethyl ether and dried in vacuo to afford the di-hydrochloride salt of the title compound (98.9 mg, 0.188 mmol, 18.20% yield) as a white solid. Lcms rt=1.04[M+H]=484.3

### Example 470

### 1-Methylethyl-2-(4-{[3-({[2-(methyloxy)phenyl]methyl}oxy)phenyl]methyl}-1-piperazinyl)-3-pyridinecarboxylate hydrochloride

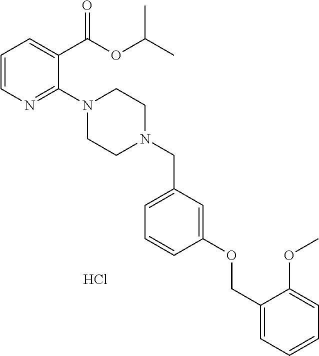 [SMILES: COc1ccccc1COc1cccc(CN2CCN(c3ncccc3C(=O)OC(C)C)CC2)c1 ; Cl]

1-Methylethyl 2-{4-[(3-hydroxyphenyl)methyl]-1-piperazinyl}-3-pyridinecarboxylate (270 mg, 0.760 mmol) and [2-(methyloxy)phenyl]methanol (157 mg, 1.139 mmol), were dissolved in dichloromethane (13.5 ml), cooled to 4° C. and then triphenylphosphine (1.44 g, 5.47 mmol) was added. The solution was stirred for 15 min while cooled to 4° C. and then di-isopropylazadicarboxylate (0.299 ml, 1.519 mmol) was added. The resulted solution was stirred at 23° C. for 24 h and then concentrated under a stream of nitrogen at 50° C. and purified by preparative hplc. (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 120 mg (33%) of the title compound free base as a clear oil. The material (120 mg, 0.25 mmol) was dissolved in di-ethyl ether 2 (mL) and treated with 2M etherial HCl (0.151 mL, 0.30 mmol) and the resulting mixture was stirred for 30 min, filtered, washed with more di-ethyl ether and dried in vacuo (0.2 mm Hg) for 24 h to afford 81 mg (21%) of the mono-hydrochloride salt of the title compound as a white solid. Lcms rt 1.02[M+H]=476.4.

### Example 471

### 1-Methylethyl-2-(4-{[3-({[4-(ethyloxy)phenyl]methyl}oxy)phenyl]methyl}-1-piperazinyl)-3-pyridinecarboxylate

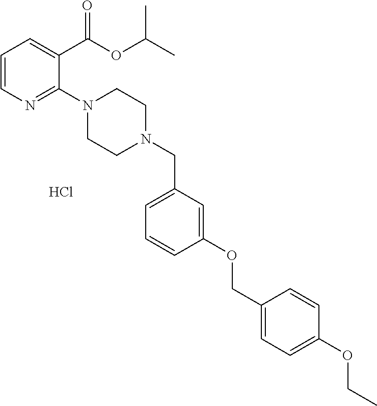 [SMILES: CCOc1ccc(COc2cccc(CN3CCN(c4ncccc4C(=O)OC(C)C)CC3)c2)cc1 ; Cl]

1-Methylethyl 2-{4-[(3-hydroxyphenyl)methyl]-1-piperazinyl}-3-pyridinecarboxylate (972 mg, 2.74 mmol) and [4-(ethyloxy)phenyl]methanol (625 mg, 4.1 mmol), were dissolved in dichloromethane (12.6 ml), cooled to 4° C. and then triphenylphosphine (1.44 g, 5.47 mmol) was added. The solution was stirred for 15 min while cooled to 4° C. and then di-isopropylazadicarboxylate (1.06 ml, 5.47 mmol) was added. The resulted solution was stirred at 23° C. for 24 h and then concentrated under a stream of nitrogen at 50° C. and purified by preparative hplc (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 215 mg (16%) of the title compound free base as a clear oil. The material (215 mg, 0.44 mmol) was dissolved in di-ethyl ether (4 mL) and treated with 2M etherial HCl (0.22 mL, 0.44 mmol) and the resulting mixture was stirred for 30 min, filtered, washed with more di-ethyl ether and dried in vacuo (0.2 mm Hg) for 24 h to afford 78 mg of the title compound mono-hydrochloride salt as a white solid. Lcms rt 1.08[M+H]=490.3.

### Example 472

### 1-Methylethyl-2-((3R)-3-{ethyl[(4′-fluoro-2-biphenylyl)methyl]amino}-1-pyrrolidinyl)-3-pyridinecarboxylate

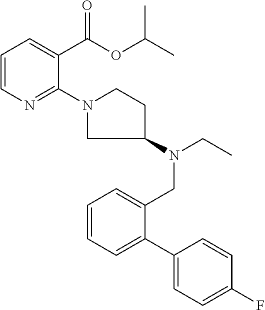 [SMILES: CCN(Cc1ccccc1-c1ccc(F)cc1)[C@@H]1CCN(c2ncccc2C(=O)OC(C)C)C1]

1-Methylethyl 2-[(3R)-3-(ethylamino)-1-pyrrolidinyl]-3-pyridinecarboxylate dihydrochloride was converted to the free base by partitioning between with EtOAc and 1 M aq NaOH. The EtOAc extract was concentrated to afford 1-Methylethyl 2-[(3R)-3-(ethylamino)-1-pyrrolidinyl]-3-pyridinecarboxylate free base as a light brown oil.

The free base 1-methylethyl 2-[(3R)-3-(ethylamino)-1-pyrrolidinyl]-3-pyridinecarboxylate (254 mg, 0.916 mmol), 4′-fluoro-2-biphenylcarbaldehyde (367 mg, 1.832 mmol), tetrahydrofuran (THF) (4.5 mL) were stirred together for 5 min and then sodium triacetoxyborohydride (582 mg, 2.75 mmol) was added. The reaction was stirred for 6 h and the reaction was diluted with EtOAc (75 mL) and washed with 1 N aq NaOH (25 mL), H₂O (25 mL) and satd aq NaCl (25 mL), dried (Na₂SO₄) and concentrated to afford a brown oil. The crude was partially purified by silica gel chromatography on a silica cartridge (12 g) eluting at 30 mL/min with a gradient running from dichloromethane to 60% EtOAc/dichloromethane over 35 min. The desired fractions were pooled, concentrated and further purified by preparative hplc (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 197 mg, 0.43 mmol (47%) of the free base of the title compound as a clear oil. The entire sample was dissolved in diethyl ether (4 mL) and 2M aq HCl in diethyl ether (0.215 mL, 0.43 mmol) was added. The resulting mixture was stirred for 30 min, filtered and the solid was washed with more diethyl ether to afford the hydrochloride salt of the title compound (145.5 mg, 0.289 mmol, 31.6% yield). Lcms rt 0.95[M+H]=462.3.

### Example 473

### 1-Methylethyl 2-[4-({4-[(ethylamino)methyl]phenyl}methyl)-1-piperazinyl]-3-pyridine carboxylate dihydrochloride

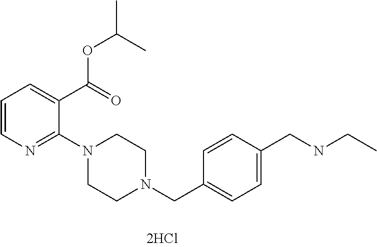 [SMILES: CCNCc1ccc(CN2CCN(c3ncccc3C(=O)OC(C)C)CC2)cc1]

1-Methylethyl2-[4-({4-[(ethylamino)methyl]phenyl}methyl)-1-piperazinyl]-3-pyridinecarboxylate (0.112 g, 0.282 mmol) was dissolved in Diethyl ether (5 mL) and 4M in dioxane hydrochloric acid (0.148 mL, 0.593 mmol) was added. The resulting mixture was stirred 20 min. Filtered, the resulting white solid was washed with more Et₂O (10 mL) and transferred to a vial and dried in vacuo (0.1 mm) for 18 h to afford the title compound (91 mg, 0.194 mmol, 68.6% yield). Lcms rt 0.70[M+H]=397.1

### Example 474

### 1-Methylethyl 2-{4-[(4-{[[(2-chloro-6-fluorophenyl)methyl](ethyl)amino]methyl}phenyl) methyl]-1-piperazinyl}-3-pyridinecarboxylate

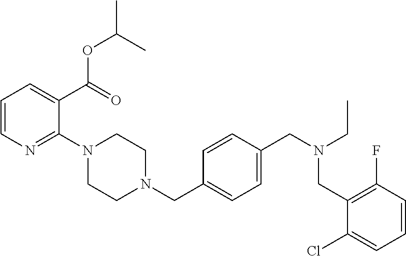 [SMILES: CCN(Cc1ccc(CN2CCN(c3ncccc3C(=O)OC(C)C)CC2)cc1)Cc1c(F)cccc1Cl]

1-Methylethyl 2-{4-[(4-formylphenyl)methyl]-1-piperazinyl}-3-pyridinecarboxylate (1.64 g, 4.46 mmol), [(2-chloro-6-fluorophenyl)methyl]ethylamine (1.005 g, 5.36 mmol), and acetic acid (0.383 ml, 6.69 mmol) were combined in 1,2-Dichloroethane (DCE) (17.47 ml) and stirred 5 min and then sodium triacetoxyborohydride (1.419 g, 6.69 mmol) was added. Stirred 16 h and diluted with dichloromethane (200 mL) and washed with 1M aq NaOH (35 mL), water (2×35 mL) and satd aq NaCl (2×35 mL), dried (Na₂SO₄) and concentrated to afford 2.53 g of a yellow oil.

Another batch of material was prepared with same reactants and reagents under similar reaction conditions as above, the product of which, was combined with above-identified 2.53 g of a yellow oil product. The combined crude material was purified.

Purification was by preparative hplc (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 1.66 (69%) of the free base as a yellow oil. LC-MS m/z=539.1 (M+H), 0.64 minutes (retention time). ¹H NMR (400 MHz, DMSO-d6) δ 8.25 (dd, J=2.01, 4.77 Hz, 1H), 7.74-7.93 (m, 1H), 7.11-7.41 (m, 7H), 6.81 (dd, J=4.52, 7.53 Hz, 1H), 5.07 (spt, J=6.23 Hz, 1H), 4.08 (q, J=5.27 Hz, 2H), 3.71 (d, J=1.25 Hz, 2H), 3.55 (s, 2H), 3.46 (s, 2H), 3.05-3.24 (m, 4H), 2.30-2.45 (m, 4H), 1.29 (d, J=6.27 Hz, 6H), 0.87-1.13 (m, 3H).

### Example 475

### 1-[(4-{[[(2-Chloro-6-fluorophenyl)methyl](ethyl)ammonio]methyl}phenyl)methyl]-4-(3-{[(1-methylethyl)oxy]carbonyl}-2-pyridinyl)piperazin-1-ium di-maleate

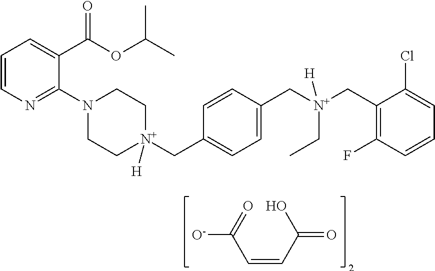

A solution of 0.1 M in ether 1-methylethyl 2-{4-[(4-{[[(2-chloro-6-fluorophenyl)methyl] (ethyl)amino]methyl}phenyl)methyl]-1-piperazinyl}-3-pyridine carboxylate (2.2 mL, 0.22 mmol) and 1.0 M in methanol maleic acid (441 μl, 0.441 mmol) were combined and diluted to 11 mL with ether and allowed to stand in the sealed vial for 3 days. Filtered to afford 1-[(4-{[[(2-chloro-6-fluorophenyl)methyl](ethyl)ammonio]methyl}phenyl)methyl]-4-(3-{[(1-methylethyl)oxy]carbonyl}-2-pyridinyl)piperazin-1-ium dimaleate (72 mg, 0.093 mmol, 42.2% yield) as light tan crystals. Microscopy with polarized light indicates the particles are birefringent. Nmr integration indicates the di-maleate. LC-MS m/z=539.4 (M+H), 0.75 minutes (retention time).

¹H NMR (400 MHz, DMSO-d₆) δ 8.35 (dd, J=1.76, 4.77 Hz, 1H), 7.99-8.08 (m, 1H), 7.26-7.49 (m, 6H), 7.17-7.25 (m, 1H), 6.91-7.02 (m, 1H), 6.15 (s, 4H), 5.05-5.16 (m, 1H), 2.87-4.41 (m, 26H), 1.30 (d, J=6.27 Hz, 6H), 1.09 (d, J=7.03 Hz, 3H).

### Example 476

### 1-methylethyl 2-{4-[(5-{[[(2-chloro-6-fluorophenyl)methyl](ethyl)amino]methyl}-2-pyridinyl)methyl]-1-piperazinyl}-3-pyridinecarboxylate

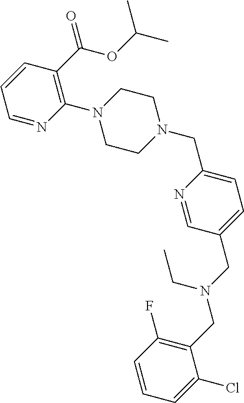 [SMILES: CCN(Cc1ccc(CN2CCN(c3ncccc3C(=O)OC(C)C)CC2)nc1)Cc1c(F)cccc1Cl]

5-({[(1,1-dimethylethyl)(dimethyl)silyl]oxy}methyl)-2-pyridinecarbaldehyde 1-Methylethyl 2-{4-[(5-formyl-2-pyridinyl)methyl]-1-piperazinyl}-3-pyridinecarboxylate (141 mg, 0.383 mmol), N-[(2-chloro-6-fluorophenyl)methyl]ethanamine (Intermediate F) (144 mg, 0.765 mmol), sodium triacetoxyborohydride (243 mg, 1.148 mmol) in THF (1.914 mL) were stirred at 23° C. for 1 day. The reaction mixture were concentrated, diluted in EtOAc, and washed with NaOH 1 N, water then brine. Purification by preparative hplc (The crude product was dissolved in DMSO (1 mL), filtered through a 0.45 μm acrodisc syringe filter, and purified on a Gilson HPLC(XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 79.2 mg (38%) of the title compound. LC-MS m/z 540.8 (M+H)⁺ 0.69 (ret time)

### Example 477

### 1-methylethyl 2-{4-[(6-{[[(2-chloro-6-fluorophenyl)methyl](ethyl)amino]methyl}-3-pyridinyl)methyl]-1-piperazinyl}-3-pyridinecarboxylate

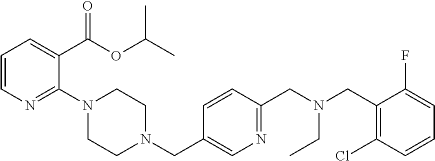 [SMILES: CCN(Cc1ccc(CN2CCN(c3ncccc3C(=O)OC(C)C)CC2)cn1)Cc1c(F)cccc1Cl]

1-Methylethyl 2-(1-piperazinyl)-3-pyridinecarboxylate (44 mg, 0.176 mmol), 6-{[[(2-chloro-6-fluorophenyl)methyl](ethyl)amino]methyl}-3-pyridinecarbaldehyde (108 mg, 0.353 mmol) and sodium triacetoxyborohydride (112 mg, 0.529 mmol) in THF (882 μl) were stirred at 23° C. for 1 day. The reaction mixture was concentrated under a stream of nitrogen at 50° C. and dissolved in EtOAc, washed with 1N aq NaOH, water then satd aq NaCl, dried (MgSO₄) and concentrated.

The residue was purified by preparative hplc (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 70.4 mg (38%) of the title compound. LC-MS m/z 540.1 (M+H)⁺ 0.67 (ret time)

### Example 478

### 1-methylethyl 2-{4-[(6-{[[(2-chloro-6-fluorophenyl)methyl](ethyl)amino]methyl}-3-pyridinyl)methyl]-1-piperazinyl}-3-pyridinecarboxylate trihydrochloride

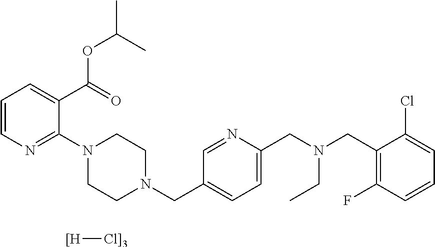

1-methylethyl 2-{4-[(6-{[[(2-chloro-6-fluorophenyl)methyl](ethyl)amino]methyl}-3-pyridinyl)methyl]-1-piperazinyl}-3-pyridinecarboxylate (74 mg, 0.137 mmol) was dissolved in diethyl ether (3 mL) and then 4M in dioxane hydrochloric acid (0.103 mL, 0.411 mmol) was added and the resulting mixture was stirred at 23° C. for 15 min and then was filtered and washed with more Et₂O to afford a hydroscopic solid. The material was transferred to a 4 mL vial and was pumped on at 0.1 mm for 14 h to afford the title compound as a dry solid. (37 mg, 42%). LC-MS m/z 540.3 (M+H)⁺ 0.74 (ret time)

### Example 479

### 1-methylethyl 2-(4-{[4-({ethyl[(2-{[(1-methylethyl)oxy]carbonyl}phenyl)methyl]amino}methyl)phenyl]methyl}-1-piperazinyl)-3-pyridinecarboxylate dihydrochloride

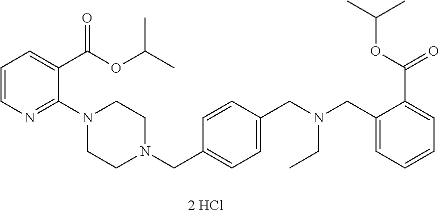 [SMILES: CCN(Cc1ccc(CN2CCN(c3ncccc3C(=O)OC(C)C)CC2)cc1)Cc1ccccc1C(=O)OC(C)C]

1-Methylethyl 2-[4-({4-[(ethylamino)methyl]phenyl}methyl)-1-piperazinyl]-3-pyridine carboxylate (313 mg, 0.789 mmol), 1-methylethyl 2-formylbenzoate (303 mg, 1.579 mmol) and sodium triacetoxyborohydride (502 mg, 2.368 mmol) in THF (4.0 mL) were stirred at 23° C. for 12 h. The volatiles were evaporated under a stream of nitrogen at 50° C. and the residue was dissolved in EtOAc washed in succession with aq 1N NaOH, water, and satd aq NaCl. The organic phase was concentrated in vacuo and the residue was purified by preparative hplc (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 225.6 mg (0.39 mmol) of the free base as a yellow oil.

The resulting product was dissolved in diethyl ether and a 2M solution of HCl in diethyl ether (0.394 mL, 0.79 mmol) was added and the resulting hydroscopic precipitate was decanted away from the diethyl ether and dissolved in H₂O and lyophilized to afford the title compound as a dry white solid. LC-MS m/z 573.6 (M+H)⁺ 0.80 (ret time).

### Example 480

### 1-methylethyl 2-(4-{[4-({ethyl[(3-{[(1-methylethyl)oxy]carbonyl}phenyl)methyl]amino}methyl)phenyl]methyl}-1-piperazinyl)-3-pyridinecarboxylate dihydrochloride

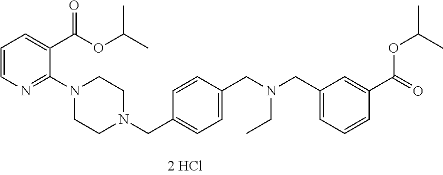 [SMILES: CCN(Cc1ccc(CN2CCN(c3ncccc3C(=O)OC(C)C)CC2)cc1)Cc1cccc(C(=O)OC(C)C)c1]

1-Methylethyl 2-[4-({4-[(ethylamino)methyl]phenyl}methyl)-1-piperazinyl]-3-pyridinecarboxylate (154 mg, 0.388 mmol) 1-methylethyl 3-formylbenzoate (74.6 mg, 0.388 mmol) and sodium triacetoxyborohydride (247 mg, 1.165 mmol) in THF (2.0 mL) were stirred at 23° C. 14 h. The volatiles were evaporated under a stream of nitrogen at 50° C. and the residue was dissolved in EtOAc washed in succession with aq 1N NaOH 1 N, water, and satd aq NaCl. The organic phase was concentrated in vacuo and the residue was purified by preparative hplc (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 38.9 mg (0.068 mmol) of the free base as a yellow oil.

The product was dissolved in diethyl ether and a 2M solution of HCl in diethyl ether (0.0678 ml, 0.136 mmol) was added and the resulting hydroscopic precipitate was decanted away from the diethyl ether and dissolved in H₂O and lyophilized to afford 31.7 mg (12%) the title compound as a brown solid. LC-MS m/z 573.6 (M+H)⁺0.81 (ret time).

### Example 481

### 1-methylethyl 2-(4-{[4-({ethyl[(4-{[(1-methylethyl)oxy]carbonyl}phenyl)methyl]amino}methyl)phenyl]methyl}-1-piperazinyl)-3-pyridinecarboxylate

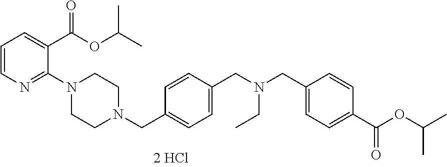 [SMILES: CCN(Cc1ccc(CN2CCN(c3ncccc3C(=O)OC(C)C)CC2)cc1)Cc1ccc(C(=O)OC(C)C)cc1]

1-Methylethyl 2-[4-({4-[(ethylamino)methyl]phenyl}methyl)-1-piperazinyl]-3-pyridinecarboxylate (197.9 mg, 0.499 mmol), 1-methylethyl 4-formylbenzoate (192 mg, 0.998 mmol) and the sodium triacetoxyborohydride (317 mg, 1.497 mmol) in THF (2.5 mL) were stirred at 23° C. for 16 h. The volatiles were evaporated under a stream of nitrogen at 50° C. and the residue was dissolved in EtOAc washed in succession with aq 1N NaOH, water and satd aq NaCl. The organic phase was concentrated in vacuo and the residue was purified by preparative hplc (The crude product was dissolved in DMSO (1 mL), filtered through a 0.45 μm acrodisc syringe filter, and purified on a Gilson HPLC (XBridge 018 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 146.39 mg (0.256 mmol) of the free base as yellow oil.

The product was dissolved in diethyl ether and a 2M solution of HCl in diethyl ether (0.26 ml, 0.72 mmol) was added and the resulting hydroscopic precipitate was decanted away from the diethyl ether and dissolved in H₂O and lyophilized to afford the di-hydrochloride salt of the title compound (101 mg, 0.156 mmol, 31.3%) as an off white solid. LC-MS m/z 573.6 (M+H)⁺ 0.84 (ret time).

### Example 482

### 1-methylethyl 2-[4-({2-[(dimethylamino)sulfonyl]phenyl}methyl)-1-piperazinyl]-3-pyridinecarboxylate hydrochloride

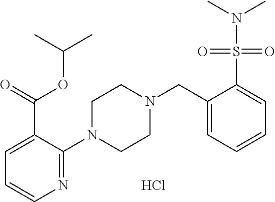 [SMILES: CC(C)OC(=O)c1cccnc1N1CCN(Cc2ccccc2S(=O)(=O)N(C)C)CC1 ; Cl]

2-Formyl-N,N-dimethylbenzenesulfonamide (145 mg, 0.682 mmol), sodium triacetoxyborohydride (217 mg, 1.023 mmol) and 1-methylethyl 2-(1-piperazinyl)-3-pyridinecarboxylate (85 mg, 0.341 mmol) in Tetrahydrofuran (THF) (2 mL) were stirred 16 h at 23° C. The reaction was concentrated under a stream of nitrogen at 50° C., extracted with EtOAc, and the organic phase was washed with aq NaOH 1 N, then water and then brine. The solvent was removed in vacuo and the residue was purified by preparative hplc (The crude product was dissolved in DMSO (1 mL), filtered through a 0.45 μm acrodisc, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford the free base as a yellow oil (81 mg, 53%) LC-MS m/z 447.3 (M+H)⁺ 0.80 (ret time).

All of the free base (81 mg, 0.182 mmol) was dissolved in diethyl ether (3 mL) and 0.091 ml of 2M HCl in diethyl ether (1 eq) was added. The resulting white solid was stirred in the ether for 30 min. More ether (2 ml) was added and the solid was loosened from the walls of the vial with a spatula. Stirred another 30 min and filtered. The white powdery solid was washed with several portions of diethyl ether and dried on high vacuum for one day and one night, to afford the title compound (55.3 mg, 0.124 mmol, 36.3% yield). Lyophilisation removed traces of solvents. LC-MS m/z 447.3 (M+H)⁺ 0.85 (ret time).

### Example 483

### 1-Methylethyl 2-[4-({3-[(dimethylamino)sulfonyl]phenyl}methyl)-1-piperazinyl]-3-pyridinecarboxylate

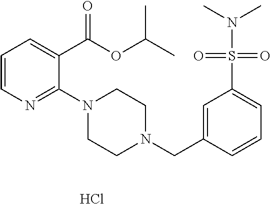 [SMILES: CC(C)OC(=O)c1cccnc1N1CCN(Cc2cccc(S(=O)(=O)N(C)C)c2)CC1 ; Cl]

Sodium triacetoxyborohydride (1.385 g, 6.53 mmol), 3-formyl-N,N-dimethylbenzenesulfonamide (0.929 g, 4.36 mmol), 1-methylethyl 2-(1-piperazinyl)-3-pyridinecarboxylate (0.543 g, 2.178 mmol) in tetrahydrofuran (THF) (10 ml) were stirred for 12 h at 23° C. Additional sodium triacetoxyborohydride (1.385 g, 6.53 mmol) was added and the reaction was stirred for another 12 h at 23° C. The reaction was concentrated under a stream of nitrogen at 50° C., extracted with EtOAc, and the organic phase was washed with aq NaOH 1 N, then water and then brine. The solvent was removed in vacuo and the residue was purified by preparative hplc. (The crude product was dissolved in DMSO (1 mL), filtered through a 0.45 μm acrodisc, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 295 mg (30%) of the free base as a yellow oil.

All of the free base (295 mg, 0.66 mmol) was dissolved in diethyl ether (5 mL), 2M HCl in diethyl ether (0.33 mL, 0.66 mmol) was added and the resulting white solid was stirred in the ether for 30 min, filtered and the solid was washed with more diethyl ether and dried in vacuo and the resulting solid was re-dissolved in water and lyophilized to remove residual solvent to afford the hydrochloride salt of 1-methylethyl 2-[4-({3-[(dimethylamino)sulfonyl]phenyl}methyl)-1-piperazinyl]-3-pyridinecarboxylate (255 mg, 24%). LC-MS m/z 447.2 (M+H)⁺ 0.76 (ret time).

### Example 484

### 1-methylethyl 2-[4-({4-[(dimethylamino)sulfonyl]phenyl}methyl)-1-piperazinyl]-3-pyridinecarboxylate

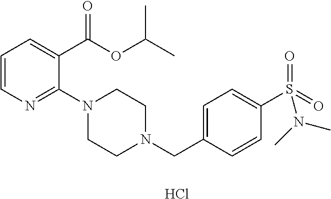 [SMILES: CC(C)OC(=O)c1cccnc1N1CCN(Cc2ccc(S(=O)(=O)N(C)C)cc2)CC1 ; Cl]

Sodium triacetoxyborohydride (1.20 g, 5.66 mmol), 4-formyl-N,N-dimethylbenzenesulfonamide (402 mg, 1.885 mmol) and the 1-methylethyl 2-(1-piperazinyl)-3-pyridinecarboxylate (470 mg, 1.89 mmol) in Tetrahydrofuran (THF) (10 ml) were stirred for 12 h at room temperature. Additional sodium triacetoxyborohydride (1.20 g, 5.66 mmol) was added and the reaction was stirred an additional 5 h. The reaction was concentrated under a stream of nitrogen at 50° C., extracted with EtOAc, and the organic phase was washed with aq NaOH 1 N, then water and then brine. The solvent was removed in vacuo and the residue was purified by preparative hplc. (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford 0.794 g (94%) of the free base as a yellow oil. All of the free base (0.794 g, 1.78 mmol) was dissolved in diethyl ether (5 mL) 0.890 ml of 2M HCl in diethyl ether (1 eq) was added and the resulting white solid was stirred in the ether for 30 min, filtered and the solid was washed with more diethyl ether and dried in vacuo and the resulting solid was re-dissolved in water and lyophilized to remove residual solvent to afford the hydrochloride salt of 1-methylethyl 2-[4-({3-[(dimethylamino)sulfonyl]phenyl}methyl)-1-piperazinyl]-3-pyridinecarboxylate (440.9 mg, 47.0%). LC-MS m/z 447.3 (M+H)⁺ 0.83 (ret time).

### Example 485

### 1-methylethyl 2-{4-[(4-{[({2-[(dimethylamino)sulfonyl]phenyl}methyl)(ethyl)amino]methyl}phenyl)methyl]-1-piperazinyl}-3-pyridinecarboxylate

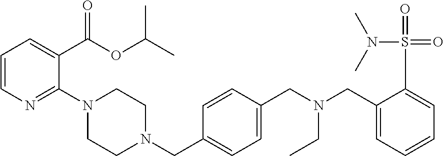 [SMILES: CCN(Cc1ccc(CN2CCN(c3ncccc3C(=O)OC(C)C)CC2)cc1)Cc1ccccc1S(=O)(=O)N(C)C]

To a solution of 1-methylethyl 2-[4-({4-[(ethylamino)methyl]phenyl}methyl)-1-piperazinyl]-3-pyridinecarboxylate (0.100 g, 0.252 mmol) and 2-formyl-N,N-dimethylbenzenesulfonamide (0.081 g, 0.378 mmol) in Methanol (5 mL) was added Acetic Acid (14 μl, 0.252 mmol) and stirred at 23° C. for 45 minutes. Then added sodium cyanoborohydride (0.050 g, 0.802 mmol) and stirred at 23° C. for 18 hours. Then added sodium cyanoborohydride (0.040 g, 0.637 mmol) and stirred at 50° C. for 6 hours. Finally added sodium cyanoborohydride (0.040 g, 0.637 mmol) and stirred at 23° C. for 2 days. The reaction was quenched with water (2 mL), the solvent was concentrated and the resulting mixture was purified by Gilson HPLC (Xbridge 19×150 mm 5u preparatory column), eluting at 18 mL/min with a linear gradient running from 70% to 90% with acetonitrile and 0.1% aqueous NH4OH over 8 minutes to give the freebase of the title compound (45 mg, 30%). LC-MS m/z=594 (M+H), 0.67 minutes (retention time).

### Example 486

### 1-methylethyl 2-{4-[(4-{[({3-[(dimethylamino)sulfonyl]phenyl}methyl)(ethyl)amino]methyl}phenyl)methyl]-1-piperazinyl}-3-pyridinecarboxylate

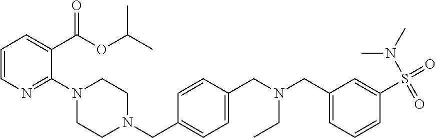 [SMILES: CCN(Cc1ccc(CN2CCN(c3ncccc3C(=O)OC(C)C)CC2)cc1)Cc1cccc(S(=O)(=O)N(C)C)c1]

To a solution of 1-methylethyl 2-[4-({4-[(ethylamino)methyl]phenyl}methyl)-1-piperazinyl]-3-pyridinecarboxylate (0.100 g, 0.252 mmol) and 3-formyl-N,N-dimethylbenzenesulfonamide (0.108 g, 0.504 mmol) in Methanol (5 mL) was added Acetic Acid (14 μl, 0.252 mmol) and stirred at 23° C. for 45 minutes. Then added sodium cyanoborohydride (0.050 g, 0.802 mmol) and stirred at 23° C. for 18 hours. Then added sodium cyanoborohydride (0.040 g, 0.637 mmol) and stirred at 50° C. for 6 hours. Finally added sodium cyanoborohydride (0.040 g, 0.637 mmol) and stirred at 23° C. for 2 days. Then added 3-formyl-N,N-dimethylbenzenesulfonamide (0.027 g, 0.126 mmol and the reaction was stirred at room temperature for 7 hours. The reaction was quenched with water (2 mL), the solvent was concentrated and the resulting mixture was purified by Gilson HPLC (Xbridge 19×150 mm 5u preparatory column), eluting at 18 mL/min with a linear gradient running from 70% to 90% with acetonitrile and 0.1% aqueous NH4OH over 8 minutes to give the freebase of the title compound. (60 mg, 40%). LC-MS m/z=594 (M+H), 0.68 minutes (retention time).

### Example 487

### 1-methylethyl 2-{4-[(4-{[({4[(dimethylamino)sulfonyl]phenyl}methyl)(ethyl)amino]methyl}phenyl)methyl]-1-piperazinyl}-3-pyridinecarboxylate

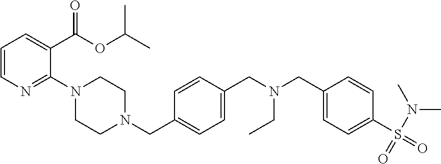 [SMILES: CCN(Cc1ccc(CN2CCN(c3ncccc3C(=O)OC(C)C)CC2)cc1)Cc1ccc(S(=O)(=O)N(C)C)cc1]

To a solution of 1-methylethyl 2-[4-({4-[(ethylamino)methyl]phenyl}methyl)-1-piperazinyl]-3-pyridinecarboxylate (0.100 g, 0.252 mmol) and 4-formyl-N,N-dimethylbenzenesulfonamide (0.108 g, 0.504 mmol) in Methanol (5 mL) was added acetic acid (14 μl, 0.252 mmol) and stirred at 23° C. for 45 minutes. Then added sodium cyanoborohydride (0.050 g, 0.802 mmol) and stirred at 23° C. for 18 hours. Then added sodium cyanoborohydride (0.040 g, 0.637 mmol) and stirred at 50° C. for 6 hours. Finally added sodium cyanoborohydride (0.040 g, 0.637 mmol) and stirred at 23° C. for 2 days. Then added 4-formyl-N,N-dimethylbenzenesulfonamide (0.027 g, 0.126 mmol and the reaction was stirred 23° C. for 7 hours. The reaction was quenched with water (2 mL), the solvent was concentrated and the resulting mixture was purified by Gilson HPLC (Xbridge 19×150 mm 5u preparatory column), eluting at 18 mL/min with a linear gradient running from 70% to 90% with acetonitrile and 0.1% aqueous NH4OH over 8 minutes to give the freebase of the title compound. (64 mg, 43%). LC-MS m/z=594 (M+H), 0.69 minutes (retention time).

### Example 488

1 1-Methylethyl 2-{4-[(4-{[[2-(2-chloro-6-fluorophenyl)ethyl](ethyl)amino]methyl}phenyl)methyl]-1-piperazinyl}-3-pyridinecarboxylate dihydrochloride

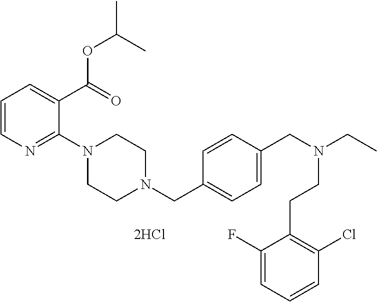 [SMILES: CCN(CCc1c(F)cccc1Cl)Cc1ccc(CN2CCN(c3ncccc3C(=O)OC(C)C)CC2)cc1]

1-Methylethyl 2-[4-({4-[(ethylamino)methyl]phenyl}methyl)-1-piperazinyl]-3-pyridinecarboxylate (0.209 g, 0.527 mmol), (2-chloro-6-fluorophenyl)acetaldehyde (0.109 g, 0.632 mmol), and tetrahydrofuran (THF) (2.078 ml) were dissolved together in a 10 mL flask under Ar, cooled on an ice bath to 4° C. and acetic acid (0.030 ml, 0.527 mmol) and sodium triacetoxyborohydride (0.168 g, 0.791 mmol) were added. The mixture was stirred 5 min and the ice bath was removed and the mixture was stirred at 23° C. for 14 h. The reaction was diluted with EtOAc (75 mL) and washed with 1 M aq NaOH (25 mL). The aq phase was washed with EtOAc (25 mL) and the combined EtOAc was washed with 1 M aq NaOH (20 mL), water (25 mL) and satd aq NaCl (25 mL), dried Na₂SO₄ and concentrated to afford a light green oil. Purification was by preparative hplc. (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 80% CH₃CN in H₂O (0.1% NH₄OH) to 100% CH₃CN over 10 min.) The desired fractions were concentrated under a stream of nitrogen at 50° C., to afford the free base of the title compound as a light yellow oil (168 mg, 58%).

The above free base (168 mg, 0.304 mmol) was dissolved in diethyl ether (5.9 mL) and 4M HCl in dioxane (152 μl, 0.607 mmol) was added. The mixture was stirred for 30 min, filtered and washed with Et₂O (3×5 mL), and dried at 0.1 mm for 4 h to afford a white solid which was dissolved in water (3 mL) and lyophillized. wt=106 mg of the title compound as the dihydrochloride (106 mg, 55.7%) as a white solid. LC-MS m/z 553.0 (M+H)⁺ 0.84 (ret time). 1H NMR (400 MHz, DMSO-d6) δ 11.75-11.94 (m, 1H), 11.51-11.70 (m, 1H), 8.29-8.43 (m, 1H), 8.00-8.12 (m, 1H), 7.81 (d, J=16.06 Hz, 4H), 7.34 (s, 2H), 7.19-7.29 (m, 1H), 6.92-7.04 (m, 1H), 5.00-5.17 (m, 1H), 4.32-4.61 (m, 4H), 3.77-3.96 (m, 2H), 2.98-3.42 (m, 11H), 1.17-1.46 (m, 9H).

### Example 489

### 1-methylethyl 2-{4-[(4-{[[(2-chloro-6-fluorophenyl)methyl](ethyl)amino]methyl}phenyl)methyl]-1-piperazinyl}-3-pyridinecarboxylate dihydrochloride

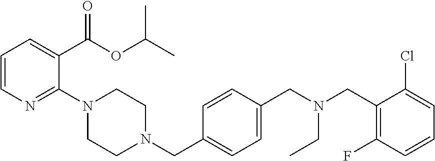 [SMILES: CCN(Cc1ccc(CN2CCN(c3ncccc3C(=O)OC(C)C)CC2)cc1)Cc1c(F)cccc1Cl]

To a solution of 1-methylethyl 2-[4-({4-[(ethylamino)methyl]phenyl}methyl)-1-piperazinyl]-3-pyridinecarboxylate (0.876 g, 2.209 mmol) in dry Methanol (15 mL) was added 2-chloro-6-fluorobenzaldehyde (0.876 g, 5.52 mmol) and acetic acid (0.025 mL, 0.442 mmol) and stirred at ambient temperature for 6 hours. Sodium cyanoborohydride (0.486 g, 7.73 mmol) was added and stirred at ambient temperature for 18 hours. Additional sodium cyanoborohydride (0.139 g, 2.209 mmol) was added and stirred for 4 hours. After which time, 2-chloro-6-fluorobenzaldehyde (0.438 g, 2.76 mmol) was added and the resulting mixture was stirred over night.

Additional 2-chloro-6-fluorobenzaldehyde (0.438 g, 2.76 mmol) and acetic acid (0.100 mL, 1.747 mmol) was added and stirred for 4 hours. Additional acetic acid (0.100 mL, 1.747 mmol) was added and stirred for approximately 4 hours. The solvent was then concentrated and the residue was dissolved in EtOAc, washed with water, and back extracted aqueous with EtOAc(2×). The combined extracts were washed with water (2×), saturated NaHCO₃, brine, dried MgSO₄, and concentrated. The resulting mixture was purified Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound (739 mg). The compound was dissolved in diethyl ether (15 mL), and 2M HCl in diethyl ether (1.326 mL, 2.65 mmol) (1.9 eq) was added and stirred for 2 hours, concentrated and dried under vacuum pump. The solid was then dissolved in 2 ml water and lyophilized to give the title compound (771 mg, 55%) as white solid. LC-MS m/z=540 (M+H), 0.69 minutes (retention time). ¹H NMR (400 MHz, DMSO-d6) δ 11.97 (br. s., 1H), 10.38 (br. s., 1H), 8.35 (dd, J=1.51, 4.52 Hz, 1H), 8.05 (dd, J=1.25, 7.53 Hz, 1H), 7.80 (s, 4H), 7.48-7.63 (m, 1H), 7.22-7.47 (m, 3H), 6.99 (dd, J=4.77, 7.53 Hz, 1H), 5.10 (dt, J=6.24, 12.36 Hz, 1H), 4.59 (br. s., 1H), 4.42 (br. s., 4H), 4.27 (br. s., 1H), 3.75-3.96 (m, 2H), 2.85-3.39 (m, 7H), 1.15-1.56 (m, 9H)

### Example 490

### 1-methylethyl 2-(4-{[4-({ethyl[(3-fluorophenyl)methyl]amino}methyl)phenyl]methyl}-1-piperazinyl)-3-pyridinecarboxylate

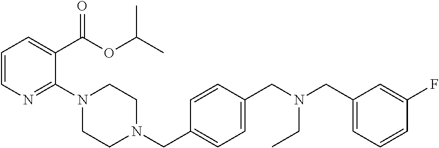 [SMILES: CCN(Cc1ccc(CN2CCN(c3ncccc3C(=O)OC(C)C)CC2)cc1)Cc1cccc(F)c1]

To a solution of 1-methylethyl 2-[4-({4-[(ethylamino)methyl]phenyl}methyl)-1-piperazinyl]-3-pyridinecarboxylate (0.100 g, 0.252 mmol) and 3-fluorobenzaldehyde (84 μl, 0.802 mmol) in Methanol (3 mL) was added acetic acid (7 μl, 0.122 mmol) and stirred at room temperature for 45 minutes. Then added sodium cyanoborohydride (0.050 g, 0.802 mmol) and stirred at room temperature for 18 hours. The solvent was concentrated and the resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (72 mg, 56%). LC-MS m/z=505 (M+H), 0.76 minutes (retention time).

### Example 491

### 1-methylethyl 2-(4-{[4-({ethyl[(4-fluorophenyl)methyl]amino}methyl)phenyl]methyl}-1-piperazinyl)-3-pyridinecarboxylate

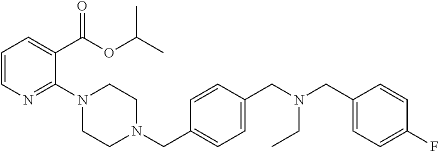 [SMILES: CCN(Cc1ccc(F)cc1)Cc1ccc(CN2CCN(c3ncccc3C(=O)OC(C)C)CC2)cc1]

To a solution of 1-methylethyl 2-[4-({4-[(ethylamino)methyl]phenyl}methyl)-1-piperazinyl]-3-pyridinecarboxylate (0.100 g, 0.252 mmol) and 4-Fluorobenzaldehyde (0.100 g, 0.802 mmol) in Methanol (3 mL) was added acetic acid (7 μl, 0.122 mmol) and stirred at room temperature for 45 minutes. Then added sodium cyanoborohydride (0.050 g, 0.802 mmol) and stirred at room temperature for 18 hours. The solvent was concentrated and the resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (74 mg, 56%). LC-MS m/z=505 (M+H), 0.75 minutes (retention time).

### Example 492

### 1-methylethyl 2-{4-[(4-{[[(2,6-difluorophenyl)methyl](ethyl)amino]methyl}phenyl)methyl]-1-piperazinyl}-3-pyridinecarboxylate

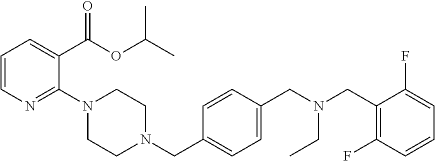 [SMILES: CCN(Cc1ccc(CN2CCN(c3ncccc3C(=O)OC(C)C)CC2)cc1)Cc1c(F)cccc1F]

To a solution of 1-methylethyl 2-[4-({4-[(ethylamino)methyl]phenyl}methyl)-1-piperazinyl]-3-pyridinecarboxylate (0.100 g, 0.252 mmol) and 2,6 difluorobenzaldehyde (0.114 g, 0.802 mmol) in Methanol (3 mL) was added acetic acid (7 μl, 0.122 mmol) and stirred at room temperature for 45 minutes. Then added sodium cyanoborohydride (0.050 g, 0.802 mmol) and stirred at room temperature for 18 hours. The solvent was concentrated and the resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (90 mg, 56%). LC-MS m/z=523 (M+H), 0.73 minutes (retention time).

### Example 493

### 1-methylethyl 2-(4-{[4-({ethyl[(2-fluorophenyl)methyl]amino}methyl)phenyl]methyl}-1-piperazinyl)-3-pyridinecarboxylate

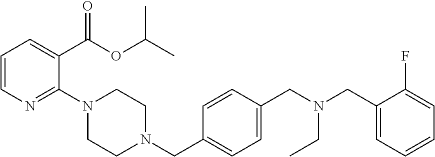 [SMILES: CCN(Cc1ccc(CN2CCN(c3ncccc3C(=O)OC(C)C)CC2)cc1)Cc1ccccc1F]

To a solution of 1-methylethyl 2-[4-({4-[(ethylamino)methyl]phenyl}methyl)-1-piperazinyl]-3-pyridinecarboxylate (0.100 g, 0.252 mmol) and 2-Fluorobenzaldehyde (65.4 μl, 0.802 mmol) in Methanol (3 mL) was added Acetic Acid (7 μl, 0.122 mmol) and stirred at room temperature for 45 minutes. Then added sodium cyanoborohydride (0.050 g, 0.802 mmol) and stirred at room temperature for 18 hours. The solvent was concentrated and the resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (72 mg, 56%). LC-MS m/z=505 (M+H), 0.72 minutes (retention time).

### Example 494

### 1-methylethyl 2-[4-({2-[(2-chloro-6-fluorophenyl)methyl]-1,2,3,4-tetrahydro-6-isoquinolinyl}methyl)-1-piperazinyl]-3-pyridinecarboxylate

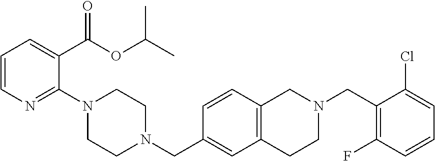 [SMILES: CC(C)OC(=O)c1cccnc1N1CCN(Cc2ccc3c(c2)CCN(Cc2c(F)cccc2Cl)C3)CC1]

To a solution of 1-methylethyl 2-[4-(1,2,3,4-tetrahydro-6-isoquinolinylmethyl)-1-piperazinyl]-3-pyridinecarboxylate-7HCl (0.165 g, 0.254 mmol) and 2-Cl, 6-F benzaldehyde (0.121 g, 0.762 mmol) in Methanol (3 mL) was stirred at room temperature for 45 minutes. Sodium cyanoborohydride (0.048 g, 0.762 mmol) was then added and the solution was stirred at room temperature for 36 hours. LCMS analysis indicated no product was formed. The solvent was concentrated and the residue was dissolved in EtOAc, and washed with 1N NaOH. The aqueous layer was backextracted with EtOAc, and the combined organics were washed with water, brine, dried MgSO₄, concentrated. This crude material was dissolved in Methanol (3 mL) and 2-Cl, 6-F benzaldehyde (0.121 g, 0.762 mmol) and acetic acid (7 μl, 0.122 mmol) were added and stirred at room temperature for 30 minutes. Then sodium cyanoborohydride (0.048 g, 0.762 mmol) was added and stirred at room temperature for 18 hours. LCMS analysis indicated title compound was major component. The solvent was concentrated and the resulting mixture was purified by Gilson HPLC (Xbridge 30×150 mm 5u preparatory column), eluting at 40 mL/min with a linear gradient running from 60% to 75% with acetonitrile and 0.1% aqueous NH₄OH over 10 minutes to give the freebase of the title compound. (63 mg, 56%) LC-MS m/z=538 (M+H), 0.65 minutes (retention time).

### Example 495

### 1-methylethyl 2-{4-[(4-{[[(2,6-dichlorophenyl)methyl}ethyl)amino]methyl}phenyl)methyl]-1-piperazinyl]-3-pyridinecarboxylate

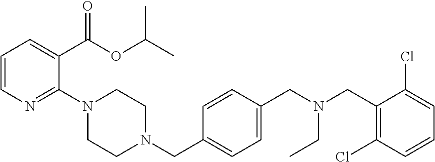 [SMILES: CCN(Cc1ccc(CN2CCN(c3ncccc3C(=O)OC(C)C)CC2)cc1)Cc1c(Cl)cccc1Cl]

To a solution of 1-methylethyl 2-[4-({4-[(ethylamino)methyl]phenyl}methyl)-1-piperazinyl]-3-pyridinecarboxylate (0.100 g, 0.252 mmol) and 2,6-dichlorobenzaldehyde (0.088 g, 0.504 mmol) in Methanol (5 mL) was added acetic acid (0.014 mL, 0.252 mmol) and stirred at room temperature for 45 minutes. Then sodium cyanoborohydride (0.050 g, 0.802 mmol) was added and stirred at room temperature for 18 hours. LCMS analysis indicated the reaction was incomplete. Additional sodium cyanoborohydride (0.050 g, 0.802 mmol) and 2,6-dichlorobenzaldehyde (0.044 g, 0.252 mmol) was added and stirred for 4 days. The solvent was concentrated and the resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (50 mg, 35%) LC-MS m/z=556 (M+H), 0.70 minutes (retention time).

### Example 496

### 1-methylethyl 2-{4-[(4-{[[(3-chlorophenyl)methyl](ethyl)amino]methyl}phenyl)methyl]-1-piperazinyl}-3-pyridinecarboxylate

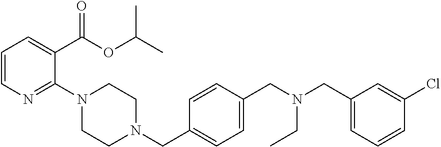 [SMILES: CCN(Cc1ccc(CN2CCN(c3ncccc3C(=O)OC(C)C)CC2)cc1)Cc1cccc(Cl)c1]

To a solution of 1-methylethyl 2-[4-({4-[(ethylamino)methyl]phenyl}methyl)-1-piperazinyl]-3-pyridinecarboxylate (0.100 g, 0.252 mmol) and 3-chlorobenzaldehyde (0.057 mL, 0.504 mmol) in Methanol (5 mL) was added acetic acid (0.014 mL, 0.252 mmol) and stirred at room temperature for 45 minutes. Then sodium cyanoborohydride (0.050 g, 0.802 mmol) was added and stirred at room temperature for 18 hours. The solvent was concentrated and the resulting mixture was purified by Gilson HPLC (Xbridge 30×150 mm 5u preparatory column), eluting at 40 mL/min with a linear gradient running from 80% to 100 with acetonitrile and 0.1% aqueous NH₄OH over 10 minutes to give the freebase of the title compound. (19 mg, 13%). LC-MS m/z=522 (M+H), 0.72 minutes (retention time).

### Example 497

### 1-methylethyl 2-{4-[(4-{[ethyl(phenylmethyl)amino]methyl}phenyl)methyl]-1-piperazinyl}-3-pyridinecarboxylate

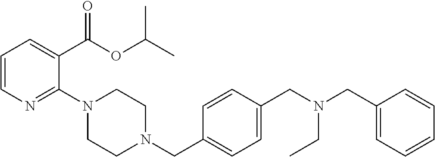 [SMILES: CCN(Cc1ccccc1)Cc1ccc(CN2CCN(c3ncccc3C(=O)OC(C)C)CC2)cc1]

To a solution of 1-methylethyl 2-[4-({4-[(ethylamino)methyl]phenyl}methyl)-1-piperazinyl]-3-pyridinecarboxylate (0.100 g, 0.252 mmol) and benzaldehyde (0.051 mL, 0.504 mmol) in methanol (5 mL) was added acetic acid (0.014 mL, 0.252 mmol) and stirred at room temperature for 45 minutes. Then sodium cyanoborohydride (0.050 g, 0.802 mmol) was added and stirred at room temperature for 18 hours. The solvent was concentrated and the resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (88 mg, 13%). LC-MS m/z=487 (M+H), 0.66 minutes (retention time).

### Example 498

### 1-methylethyl 2-{4-[(4-{[[(4-chlorophenyl)methyl](ethyl)amino]methyl}phenyl)methyl]-1-piperazinyl}-3-pyridinecarboxylate

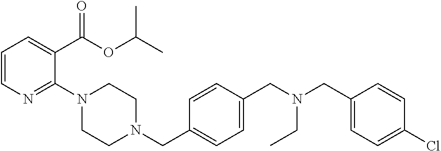 [SMILES: CCN(Cc1ccc(Cl)cc1)Cc1ccc(CN2CCN(c3ncccc3C(=O)OC(C)C)CC2)cc1]

To a solution of 1-methylethyl 2-[4-({4-[(ethylamino)methyl]phenyl}methyl)-1-piperazinyl]-3-pyridinecarboxylate (0.100 g, 0.252 mmol) and 4-chlorobenzaldehyde (0.071 g, 0.504 mmol) in methanol (5 mL) was added acetic acid (0.014 mL, 0.252 mmol) and stirred at room temperature for 45 minutes. Then sodium cyanoborohydride (0.050 g, 0.802 mmol) was added and stirred at room temperature for 18 hours. The solvent was concentrated and the resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (83 mg, 13%). LC-MS m/z=522 (M+H), 0.73 minutes (retention time).

### Example 499

### 1-methylethyl 2-{4-[(4-{[[(2-chlorophenyl)methyl](ethyl)amino]methyl}phenyl)methyl]-1-piperazinyl}-3-pyridinecarboxylate

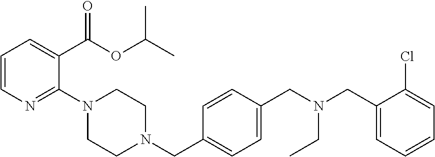 [SMILES: CCN(Cc1ccc(CN2CCN(c3ncccc3C(=O)OC(C)C)CC2)cc1)Cc1ccccc1Cl]

To a solution of 1-methylethyl 2-[4-({4-[(ethylamino)methyl]phenyl}methyl)-1-piperazinyl]-3-pyridinecarboxylate (0.100 g, 0.252 mmol) and 2-chlorobenzaldehyde (0.057 mL, 0.504 mmol) in Methanol (5 mL) was added Acetic Acid (0.014 mL, 0.252 mmol) and stirred at room temperature for 45 minutes. Then sodium cyanoborohydride (0.050 g, 0.802 mmol) was added and stirred at room temperature for 18 hours. The solvent was concentrated and the resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (87 mg, 13%). LC-MS m/z=522 (M+H), 0.68 minutes (retention time).

### Example 500

### 1-methylethyl 2-(4-{[4-({ethyl[(6-methyl-2-pyridinyl)methyl]amino}methyl)phenyl]methyl}-1-piperazinyl)-3-pyridinecarboxylate

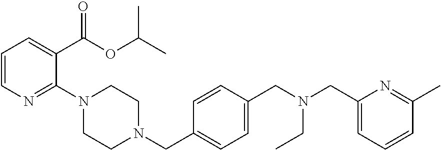 [SMILES: CCN(Cc1ccc(CN2CCN(c3ncccc3C(=O)OC(C)C)CC2)cc1)Cc1cccc(C)n1]

To a solution of 1-methylethyl 2-[4-({4-[(ethylamino)methyl]phenyl}methyl)-1-piperazinyl]-3-pyridinecarboxylate (0.100 g, 0.252 mmol) and 6-methyl-2-pyridinecarbaldehyde (0.031 g, 0.252 mmol) in Methanol (3 mL) was added Acetic Acid (7.22 μL, 0.126 mmol) and stirred at room temperature for 45 minutes. Then sodium cyanoborohydride (0.024 g, 0.378 mmol) was added and stirred at room temperature for 18 hours. The solvent was concentrated and the resulting mixture was purified by Gilson HPLC (Xbridge 30×150 mm 5u preparatory column), eluting at 40 mL/min with a linear gradient running from 75% to 95% with acetonitrile and 0.1% aqueous NH₄O H over 10 minutes to give the freebase of the title compound. (30 mg, 13%). LC-MS m/z=501 (M+H), 0.78 minutes (retention time).

### Example 501

### 1-methylethyl 2-(4-{[4-({[(2-chloro-6-fluorophenyl)methyl]amino}methyl)phenyl]methyl}-1-piperazinyl)-3-pyridinecarboxylate

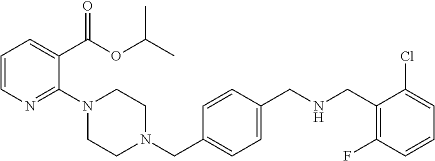 [SMILES: CC(C)OC(=O)c1cccnc1N1CCN(Cc2ccc(CNCc3c(F)cccc3Cl)cc2)CC1]

To a solution of 1-methylethyl 2-{4-[(4-formylphenyl)methyl]-1-piperazinyl}-3-pyridinecarboxylate (0.100 g, 0.272 mmol) and 2-Chloro-6-fluorobenzylamine (0.065 g, 0.408 mmol) in Methanol (3 mL) was added acetic acid (0.016 ml, 0.272 mmol) and stirred at room temperature for 1 hour. Then sodium cyanoborohydride (0.051 g, 0.816 mmol) was added and stirred at room temperature for 18 hours. The solvent was concentrated and the resulting mixture was purified by Gilson HPLC (Xbridge 30×150 mm 5u preparatory column), eluting at 40 mL/min with a linear gradient running from 75 to 95% with acetonitrile and 0.1% aqueous NH₄OH over 10 minutes to give the freebase of the title compound. (103 mg, 74%). LC-MS m/z=511 (M+H), 0.76 minutes (retention time).

### Example 502

### 1-methylethyl 2-{4-[(4-{[[(2-chloro-6-fluorophenyl)carbonyl](ethyl)amino]methyl}phenyl)methyl]-1-piperazinyl}-3-pyridinecarboxylate

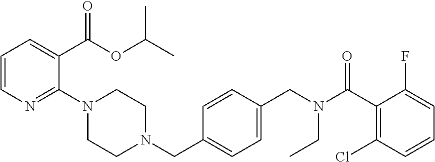 [SMILES: CCN(Cc1ccc(CN2CCN(c3ncccc3C(=O)OC(C)C)CC2)cc1)C(=O)c1c(F)cccc1Cl]

To a solution of 2-chloro-6-fluorobenzoic acid (0.106 g, 0.605 mmol) in Dichloromethane (DCM) (10 mL) was added oxalyl chloride (0.158 mL, 1.80 mmol) and followed by 1 drop of DMF and stirred at room temperature for 2 hours. The solvent was concentrated; the excess oxalyl chloride was azeotroped with DCM and the acid chloride was pumped on high vacuum. In separate 20 ml vial, 1-methylethyl 2-[4-({4-[(ethylamino)methyl]phenyl}methyl)-1-piperazinyl]-3-pyridinecarboxylate (0.100 g, 0.252 mmol) was dissolved in acetonitrile (7 mL). 2,4 Lutidine (0.208 mL, 1.80 mmol) was added and stirred in an ice bath. The acid chloride was then dissolved in acetonitrile (3 mL) and added to the solution of amine. The green reaction was stirred at room temperature for 18 hours. The solvent was concentrated, and the residue was dissolved in EtOAc and saturated NaHCO₃. The layers were separated and the aqueous layer was backextracted with EtOAc, washed combined organics with sat NaHCO₃, dried MgSO₄, and concentrated. The resulting mixture was purified by Gilson HPLC (Xbridge 30×150 mm 5u preparatory column), eluting at 40 mL/min with a linear gradient running from 60% to 75% with acetonitrile and 0.1% aqueous NH₄OH over 10 minutes to give the freebase of the title compound which was an inseparable mixture of cis and trans amide isomers. (74 mg, 53%). Isomer A was 32% of the mixture and had LC-MS m/z=553 (M+H), 0.87 minutes (retention time). Isomer B was 68% of the mixture and had LC-MS m/z=553 (M+H), 0.90 minutes (retention time).

### Example 503

### 1-methylethyl 2-{(3R)-3-[[(4-{[[(2-chloro-6-fluorophenyl)methyl](ethyl)amino]methyl}phenyl)methyl](ethyl)amino]-1-pyrrolidinyl}-3-pyridinecarboxylate

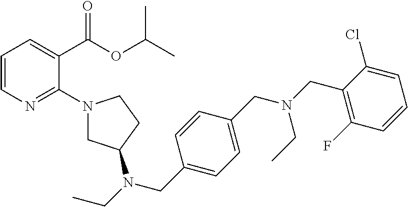 [SMILES: CCN(Cc1ccc(CN(CC)[C@@H]2CCN(c3ncccc3C(=O)OC(C)C)C2)cc1)Cc1c(F)cccc1Cl]

To a solution of 1-methylethyl 2-{(3R)-3-[ethyl({4-[(ethylamino)methyl]phenyl}methyl)amino]-1-pyrrolidinyl}-3-pyridinecarboxylate (0.077 g, 0.181 mmol) in Methanol (1.803 ml) was added 2-Cl,6-Fl-benzaldehyde (0.058 g, 0.363 mmol) and acetic acid (10.38 μl, 0.181 mmol) and stirred at room temperature for 1 hour. Then sodium cyanoborohydride (0.034 g, 0.544 mmol) was added and stirred at room temperature for 18 hours. Afterwards, additional 2-Cl,6-Fl-benzaldehyde (0.029 g, 0.181 mmol) and sodium cyanoborohydride (0.011 g, 0.181 mmol) were added and stirred at room temperature for 2 days. The reaction was quenched with 1 ml water, and concentrated the solvent. The residue was dissolved in EtOAc, washed with water (2×), brine, dried MgSO₄. The resulting mixture was purified by Gilson HPLC (Xbridge 30×150 mm 5u preparatory column), eluting at 40 mL/min with a linear gradient running from 80 to 100% with acetonitrile and 0.1% aqueous NH₄OH over 10 minutes to give the freebase of the title compound. (32 mg, 31%). LC-MS m/z=568 (M+H), 0.71 minutes (retention time).

### Example 504

### 1-methylethyl 2-(4-{[4-({[(2-chloro-6-fluorophenyl)methyl][3-(2-oxo-1-pyrrolidinyl) propyl]amino}methyl)phenyl]methyl}-1-piperazinyl)-3-pyridinecarboxylate

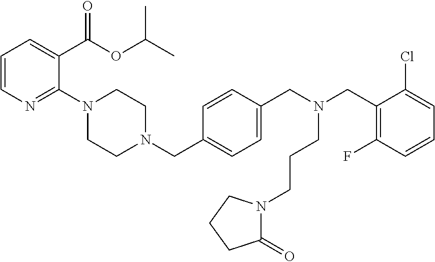 [SMILES: CC(C)OC(=O)c1cccnc1N1CCN(Cc2ccc(CN(CCCN3CCCC3=O)Cc3c(F)cccc3Cl)cc2)CC1]

To a solution of 1-methylethyl 2-{4-[(4-formylphenyl)methyl]-1-piperazinyl}-3-pyridinecarboxylate (0.065 g, 0.177 mmol) in 1,2-dichloroethane (DCE) (3 ml) was added 1-(3-{[(2-chloro-6-fluorophenyl)methyl]amino}propyl)-2-pyrrolidinone (0.060 g, 0.212 mmol) and then Acetic Acid (0.015 ml, 0.265 mmol) and stirred at room temperature for 10 minutes. Then sodium triacetoxyborohydride (0.056 g, 0.265 mmol) was added and stirred at room temperature for 1 hour. The reaction was diluted with DCM and washed with 1N NaOH. The layers were separated on a hydrophobic phase separator column, and the aqueous layer was extracted with DCM. The layers were separated as above, and the organic layer was concentrated. The resulting mixture was purified by Gilson HPLC (Xbridge 30×150 mm 5u preparatory column), eluting at 40 mL/min with a linear gradient running from 65% to 85% with acetonitrile and 0.1% aqueous NH₄OH over 10 minutes to give the freebase of the title compound. (57 mg, 50%). LC-MS m/z=637 (M+H), 0.70 minutes (retention time).

### Example 505

### (2-{4-[(4-{[[(2-chloro-6-fluorophenyl)methyl](ethyl)amino]methyl}phenyl)methyl]-1-piperazinyl}-3-pyridinyl)methyl 2,2,3,3-tetramethylcyclopropanecarboxylate

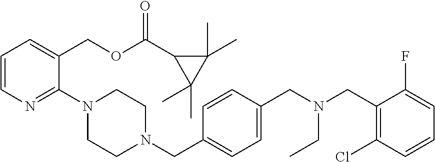 [SMILES: CCN(Cc1ccc(CN2CCN(c3ncccc3COC(=O)C3C(C)(C)C3(C)C)CC2)cc1)Cc1c(F)cccc1Cl]

To a solution of 2,2,3,3-tetramethylcyclopropanecarboxylic acid (0.015 g, 0.109 mmol) in dichloromethane (DCM) (1 mL) was added oxalyl chloride (10.87 μL, 0.124 mmol) and stirred at room temperature for 20 hours. In a separate flask, was dissolved (2-{4-[(4-{[[(2-chloro-6-fluorophenyl)methyl](ethyl)amino]methyl}phenyl)methyl]-1-piperazinyl}-3-pyridinyl)methanol (0.050 g, 0.104 mmol) in Dichloromethane (DCM) (2 mL). Triethylamine (0.020 mL, 0.145 mmol) was added to the solution which was then cooled to 0° C. The acid chloride solution was transferred to the cooled solution via pipette and stirred at room temperature for 20 hours. LCMS analysis indicated the presence of unreacted starting material alcohol, so another batch of acid chloride was prepared by dissolving 2,2,3,3-tetramethylcyclopropanecarboxylic acid (0.015 g, 0.109 mmol) in dichloromethane (DCM) (1 mL) and adding oxalyl chloride (10.87 μL, 0.124 mmol) which was stirred at room temperature for 2 hours. The solution was then added directly to reaction and stirred for 20 hours. Methanol was added to quench any excess acid chloride present and all the solvents were concentrated. The resulting mixture was purified by Gilson HPLC (Xbridge 19×150 mm 5u preparatory column), eluting at 18 mL/min with a linear gradient running from 80% to 100% with acetonitrile and 0.1% aqueous NH₄OH over 10 minutes to give the freebase of the title compound. (19 mg, 50%). LC-MS m/z=607 (M+H), 0.64 minutes (retention time).

### Example 506

### (2-{4-[(4-{[[(2-chloro-6-fluorophenyl)methyl](ethyl)amino]methyl}phenyl)methyl]-1-piperazinyl}-3-pyridinyl)methyl 3,3-dimethylbutanoate

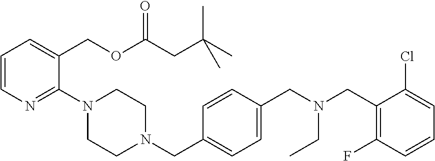 [SMILES: CCN(Cc1ccc(CN2CCN(c3ncccc3COC(=O)CC(C)(C)C)CC2)cc1)Cc1c(F)cccc1Cl]

To a solution of (2-{4-[(4-{[[(2-chloro-6-fluorophenyl)methyl](ethyl)amino]methyl}phenyl)methyl]-1-piperazinyl}-3-pyridinyl)methanol (0.050 g, 0.104 mmol) in dichloromethane (DCM) (2.00 mL) was added triethylamine (0.029 mL, 0.207 mmol) and cooled in ice bath. Then t-butylacetyl chloride (0.016 mL, 0.114 mmol) was added and stirred at room temperature for 20 hours. LCMS analysis indicated the presence of starting material alcohol. Additional t-butylacetyl chloride (0.016 mL, 0.114 mmol) was added to reaction and stirred at room temperature for 4 hours. Methanol was added to quench any excess acid chloride present and all the solvents were concentrated. The resulting mixture was purified by Gilson HPLC (Xbridge 19×150 mm 5u preparatory column), eluting at 18 mL/min with a linear gradient running from 80% to 100% with acetonitrile and 0.1% aqueous NH₄OH over 10 minutes to give the freebase of the title compound. (47 mg, 78%). LC-MS m/z=581 (M+H), 0.82 minutes (retention time).

### Example 507

### (2-{4-[(4-{[[(2-chloro-6-fluorophenyl)methyl](ethyl)amino]methyl}phenyl)methyl]-1-piperazinyl}-3-pyridinyl)methyl 2-methylpropanoate

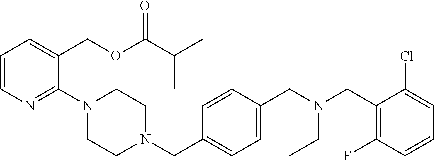 [SMILES: CCN(Cc1ccc(CN2CCN(c3ncccc3COC(=O)C(C)C)CC2)cc1)Cc1c(F)cccc1Cl]

To a solution of (2-{4-[(4-{[[(2-chloro-6-fluorophenyl)methyl](ethyl)amino]methyl}phenyl)methyl]-1-piperazinyl}-3-pyridinyl)methanol (0.050 g, 0.104 mmol) in dichloromethane (DCM) (2.00 mL) was added triethylamine (0.029 mL, 0.207 mmol) and cooled in ice bath. Then isobutyryl chloride (0.012 mL, 0.114 mmol) was added and stirred at room temperature for 20 hours. LCMS analysis indicated the presence of starting material alcohol. Additional isobutyryl chloride (0.012 mL, 0.114 mmol) was added to reaction and stirred at room temperature for 4 hours. Methanol was added to quench any excess acid chloride present and all the solvents were concentrated. The resulting mixture was purified by Gilson HPLC (Xbridge 19×150 mm 5u preparatory column), eluting at 18 mL/min with a linear gradient running from 80% to 100% with acetonitrile and 0.1% aqueous NH₄OH over 10 minutes to give the freebase of the title compound. (47 mg, 78%). LC-MS m/z=553 (M+H), 0.69 minutes (retention time).

### Example 508

### (2-{4-[(4-{[[(2-chloro-6-fluorophenyl)methyl](ethyl)amino]methyl}phenyl)methyl]-1-piperazinyl}-3-pyridinyl)methyl acetate

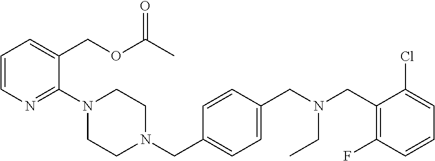 [SMILES: CCN(Cc1ccc(CN2CCN(c3ncccc3COC(C)=O)CC2)cc1)Cc1c(F)cccc1Cl]

To a solution of (2-{4-[(4-{[[(2-chloro-6-fluorophenyl)methyl](ethyl)amino]methyl}phenyl)methyl]-1-piperazinyl}-3-pyridinyl)methanol (0.050 g, 0.104 mmol) in dichloromethane (DCM) (2.00 mL) was added triethylamine (0.029 mL, 0.207 mmol) and cooled in ice bath. Then acetyl chloride (8.10 μL, 0.114 mmol) was added and stirred at room temperature for 20 hours. LCMS analysis indicated the presence of starting material alcohol. Additional acetyl chloride (8.10 μL, 0.114 mmol) was added to reaction and stirred at room temperature for 4 hours. Methanol was added to quench any excess acid chloride present and all the solvents were concentrated. The resulting mixture was purified by Gilson HPLC (Xbridge 19×150 mm 5u preparatory column), eluting at 18 mL/min with a linear gradient running from 80% to 100% with acetonitrile and 0.1% aqueous NH₄OH over 10 minutes to give the freebase of the title compound. (43 mg, 79%). LC-MS m/z=525 (M+H), 0.62 minutes (retention time).

### Example 509

### 1-methylethyl 2-{4-[(5-{[[(2-chloro-6-fluorophenyl)methyl](ethyl)amino]methyl}-2-pyrazinyl)methyl]-1-piperazinyl}-3-pyridinecarboxylate

### Example 510 470

### bis(1-methylethyl) 2,2′-[2,5-pyrazinediylbis(methanediyl-4,1-piperazinediyl)]di(3-pyridine carboxylate)

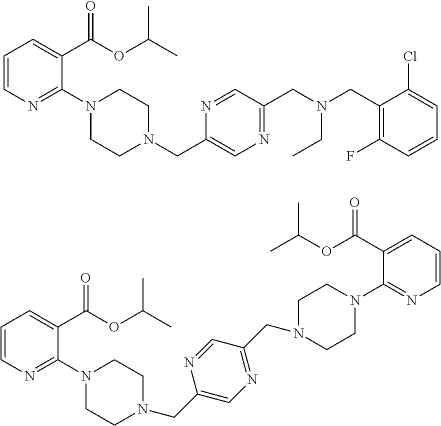 [SMILES: CCN(Cc1cnc(CN2CCN(c3ncccc3C(=O)OC(C)C)CC2)cn1)Cc1c(F)cccc1Cl ; CC(C)OC(=O)c1cccnc1N1CCN(Cc2cnc(CN3CCN(c4ncccc4C(=O)OC(C)C)CC3)cn2)CC1]

To a solution of 1-methylethyl 2-(1-piperazinyl)-3-pyridinecarboxylate (0.050 g, 0.201 mmol), 2,5-bis(bromomethyl)pyrazine (0.053 g, 0.201 mmol) and [(2-chloro-6-fluorophenyl)methyl]ethylamine (0.038 g, 0.201 mmol) in Acetone (4 mL) was added K2CO3 (0.055 g, 0.401 mmol) and heated to 50° C. for 4 hours and then stirred at room temperature for 18 hours. The reaction was then filtered and solvent concentrated. The resulting mixture was purified by Gilson HPLC (Xbridge 19×150 mm 5u preparatory column), eluting at 18 mL/min with a linear gradient running from 50% to 100% with acetonitrile and 0.1% aqueous NH₄OH over 20 minutes to give the freebase of:
=1-methylethyl 2-{4-[(5-{[[(2-chloro-6-fluorophenyl)methyl](ethyl)amino]methyl}-2-pyrazinyl)methyl]-1-piperazinyl}-3-pyridinecarboxylate (29 mg, 26%). LC-MS m/z=541 (M+H), 0.69 minutes (retention time).
=bis(1-methylethyl) 2,2′-[2,5-pyrazinediylbis(methanediyl-4,1-piperazinediyl)]di(3-pyridinecarboxylate)
   (24 mg, 19%). LC-MS m/z=602 (M+H), 0.74 minutes (retention time).

### Example 511

### (2-{4-[(4-{[[(2-chloro-6-fluorophenyl)methyl](ethyl)amino]methyl}phenyl)methyl]-1-piperazinyl}-3-pyridinyl)methyl cyclopropanecarboxylate

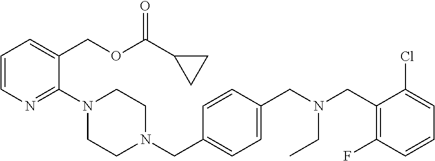 [SMILES: CCN(Cc1ccc(CN2CCN(c3ncccc3COC(=O)C3CC3)CC2)cc1)Cc1c(F)cccc1Cl]

To a solution of (2-{4-[(4-{[[(2-chloro-6-fluorophenyl)methyl](ethyl)amino]methyl}phenyl)methyl]-1-piperazinyl}-3-pyridinyl)methanol (0.050 g, 0.104 mmol) in Dichloromethane (DCM) (2.00 mL) was added triethylamine (0.029 mL, 0.207 mmol) and cooled in ice bath. Then cyclopropanecarbonyl chloride (10.33 μL, 0.114 mmol) was added and stirred at room temperature for 20 hours. LCMS analysis indicated the presence of starting material alcohol. Additional cyclopropanecarbonyl chloride (10.33 μL, 0.114 mmol) was added to reaction and stirred at room temperature for 4 hours. Methanol was added to quench any excess acid chloride present and all the solvents were concentrated. The resulting mixture was purified by Gilson HPLC (Xbridge 19×150 mm 5u preparatory column), eluting at 18 mL/min with a linear gradient running from 80% to 100% with acetonitrile and 0.1% aqueous NH₄OH over 10 minutes to give the freebase of the title compound. (37 mg, 64%). LC-MS m/z=551 (M+H), 0.67 minutes (retention time).

### Example 512

### 1-methylethyl 2-{4-[(3-{[[(2-chloro-6-fluorophenyl)methyl](ethyl)amino]methyl}phenyl)methyl]-1-piperazinyl}-3-pyridinecarboxylate

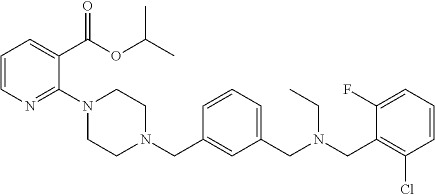 [SMILES: CCN(Cc1cccc(CN2CCN(c3ncccc3C(=O)OC(C)C)CC2)c1)Cc1c(F)cccc1Cl]

To a solution of 1-methylethyl 2-{4-[(3-formylphenyl)methyl]-1-piperazinyl}-3-pyridinecarboxylate (0.100 g, 0.272 mmol) in 1,2-Dichloroethane (DCE) (15 ml) was added N-[(2-chloro-6-fluorophenyl)methyl]ethanamine (0.061 g, 0.327 mmol) and then Acetic Acid (0.019 ml, 0.327 mmol) and stirred at room temperature for 10 minutes. Sodium triacetoxyborohydride (0.087 g, 0.408 mmol) was added and the reaction was stirred at room temperature for 2 days. The reaction was then quenched with 1 ml water and all solvents were concentrated. The resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (108 mg, 73%). LC-MS m/z=539 (M+H), 0.67 minutes (retention time).

### Example 513

### (2-{4-[(4-{[[(2-chloro-6-fluorophenyl)methyl](ethyl)amino]methyl}phenyl)methyl]-1-piperazinyl}-3-pyridinyl)methyl propanoate

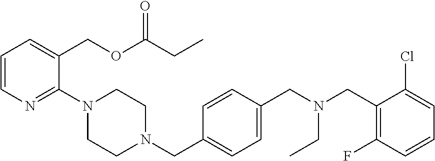 [SMILES: CCC(=O)OCc1cccnc1N1CCN(Cc2ccc(CN(CC)Cc3c(F)cccc3Cl)cc2)CC1]

To a solution of (2-{4-[(4-{[[(2-chloro-6-fluorophenyl)methyl](ethyl)amino]methyl}phenyl)methyl]-1-piperazinyl}-3-pyridinyl)methanol (0.050 g, 0.104 mmol) in Dichloromethane (DCM) (2.00 mL) was added triethylamine (0.029 mL, 0.207 mmol) and cooled in ice bath. Then propionyl chloride (9.89 μL, 0.114 mmol) was added and stirred at room temperature for 20 hours. LCMS analysis indicated the presence of starting material alcohol. Additional propionyl chloride (9.89 μL, 0.114 mmol) was added to reaction and stirred at room temperature for 4 hours. Methanol was added to quench any excess acid chloride present and all the solvents were concentrated. The resulting mixture was purified by Gilson HPLC (Xbridge 19×150 mm 5u preparatory column), eluting at 18 mL/min with a linear gradient running from 80% to 100% with acetonitrile and 0.1% aqueous NH₄OH over 10 minutes to give the freebase of the title compound. (46 mg, 82%). LC-MS m/z=539 (M+H), 0.66 minutes (retention time).

### Example 514

### 1-methylethyl 2-(4-{[4-({ethyl[(2-methyl-3-pyridinyl)methyl]amino}methyl)phenyl]methyl}-1-piperazinyl)-3-pyridinecarboxylate

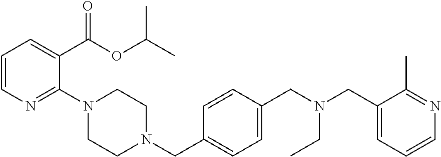 [SMILES: CCN(Cc1ccc(CN2CCN(c3ncccc3C(=O)OC(C)C)CC2)cc1)Cc1cccnc1C]

To a solution of 1-methylethyl 2-[4-({4-[(ethylamino)methyl]phenyl}methyl)-1-piperazinyl]-3-pyridinecarboxylate (0.100 g, 0.252 mmol) and 2-methyl-3-pyridinecarbaldehyde (0.015 g, 0.126 mmol) in 1,2-Dichloroethane (DCE) (3 mL) was added Acetic Acid (7.22 μL, 0.126 mmol) and stirred at room temperature for 45 minutes. Then sodium triacetoxyborohydride (0.053 g, 0.252 mmol) was added and stirred at room temperature for 18 hours. The reaction was then quenched with 1 ml water and the solvents were concentrated. The resulting mixture was purified by Gilson HPLC (Xbridge 19×150 mm 5u preparatory column), eluting at 18 mL/min with a linear gradient running from 70% to 100% with acetonitrile and 0.1% aqueous NH₄OH over 10 minutes to give the freebase of the title compound. (16 mg, 25%). LC-MS m/z=502 (M+H), 0.61 minutes (retention time).

### Example 515

### 1-methylethyl2-(4-{[4-({[(2-fluorophenyl)methyl]amino}methyl)phenyl]methyl}-1-piperazinyl)-3-pyridinecarboxylate

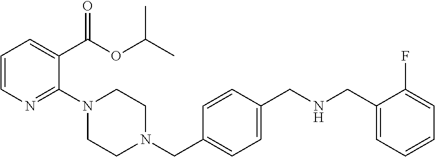 [SMILES: CC(C)OC(=O)c1cccnc1N1CCN(Cc2ccc(CNCc3ccccc3F)cc2)CC1]

To a solution of 1-methylethyl 2-{4-[(4-formylphenyl)methyl]-1-piperazinyl}-3-pyridinecarboxylate (0.100 g, 0.272 mmol) and 2-Fluorobenzylamine (0.034 g, 0.272 mmol) in Methanol (3 mL) was added acetic acid (0.016 ml, 0.272 mmol) and stirred at room temperature for 1 hour. Then sodium cyanoborohydride (0.051 g, 0.816 mmol) was added and stirred at room temperature for 21 hours. The solvent was concentrated and the resulting mixture was purified by Gilson HPLC (Xbridge 19×150 mm 5u preparatory column), eluting at 18 mL/min with a linear gradient running from 75% to 95% with acetonitrile and 0.1% aqueous NH₄OH over 10 minutes to give the freebase of the title compound. (68 mg, 52%). LC-MS m/z=477 (M+H), 0.64 minutes (retention time).

### Example 516

### 1-methylethyl 2-{4-[(2-{[[(2-chloro-6-fluorophenyl)methyl](ethyl)amino]methyl}phenyl)methyl]-1-piperazinyl}-3-pyridinecarboxylate

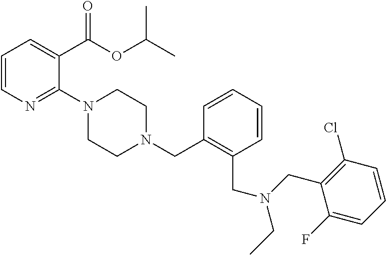 [SMILES: CCN(Cc1ccccc1CN1CCN(c2ncccc2C(=O)OC(C)C)CC1)Cc1c(F)cccc1Cl]

To a solution of 1-methylethyl 2-{4-[(2-formyl phenyl)methyl]-1-piperazinyl}-3-pyridinecarboxylate (0.046 g, 0.125 mmol) in 1,2-Dichloroethane (DCE) (7 ml) was added N-[(2-chloro-6-fluorophenyl)methyl]ethanamine (0.028 g, 0.150 mmol) and then acetic acid (8.60 μl, 0.150 mmol) and stirred at room temperature for 10 minutes. Sodium triacetoxyborohydride (0.040 g, 0.188 mmol) was added and stirred at room temperature for 20 hours. LCMS analysis indicated presence of unreacted aldehyde starting material. Additional sodium triacetoxyborohydride (0.040 g, 0.188 mmol) was added and the reaction was stirred at room temperature for 66 hours. The reaction was quenched with 1 ml water and all solvents were concentrated. The resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (10 mg, 14%). LC-MS m/z=539 (M+H), 0.96 minutes (retention time).

### Example 517

### 1-methylethyl2-{4-[(4-{[[3-(2-chloro-6-fluorophenyl)propyl](ethyl)amino]methyl}phenyl)methyl]-1-piperazinyl}-3-pyridinecarboxylate

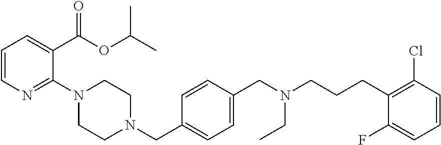 [SMILES: CCN(CCCc1c(F)cccc1Cl)Cc1ccc(CN2CCN(c3ncccc3C(=O)OC(C)C)CC2)cc1]

To a solution of 1-methylethyl 2-[4-({4-[(ethylamino)methyl]phenyl}methyl)-1-piperazinyl]-3-pyridinecarboxylate (0.066 g, 0.166 mmol) and 3-(2-chloro-6-fluorophenyl)propanal (0.047 g, 0.250 mmol) in methanol (3 mL) was added acetic acid (9.53 μL, 0.166 mmol) and stirred at room temperature for 45 minutes. Then sodium cyanoborohydride (0.026 g, 0.416 mmol) was added and stirred at room temperature for 66 hours. The reaction was quenched with 1 ml water, the solvent was concentrated and the resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (65 mg, 14% for two reactions). LC-MS m/z=567 (M+H), 0.82 minutes (retention time).

### Example 518

### 1-methylethyl 2-{4-[(4-{[(phenylmethyl)amino]methyl}phenyl)methyl]-1-piperazinyl}-3-pyridinecarboxylate

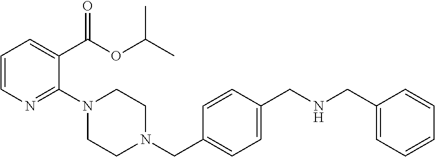 [SMILES: CC(C)OC(=O)c1cccnc1N1CCN(Cc2ccc(CNCc3ccccc3)cc2)CC1]

To a solution of 1-methylethyl 2-{4-[(4-formylphenyl)methyl]-1-piperazinyl}-3-pyridinecarboxylate (0.100 g, 0.272 mmol) and benzylamine (0.030 ml, 0.272 mmol) in methanol (3 mL) was added acetic acid (0.016 ml, 0.272 mmol) and stirred at room temperature for 1 hour. Then sodium cyanoborohydride (0.051 g, 0.816 mmol) was added and stirred at room temperature for 18 hours. The solvent was concentrated and the resulting mixture was purified by Gilson HPLC (Xbridge 30×150 mm 5u preparatory column), eluting at 40 mL/min with a linear gradient running from 65% to 85% with acetonitrile and 0.1% aqueous NH₄OH over 10 minutes to give the freebase of the title compound. (65 mg, 52%). LC-MS m/z=459 (M+H), 0.66 minutes (retention time).

### Example 519

### 1-methylethyl 2-(4-{[4-({ethyl[(2-fluorophenyl)methyl]amino}methyl)phenyl]methyl}-1-piperazinyl)-3-pyridinecarboxylate dihydrochloride

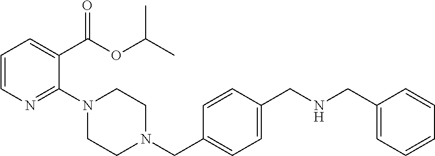 [SMILES: CC(C)OC(=O)c1cccnc1N1CCN(Cc2ccc(CNCc3ccccc3)cc2)CC1]

To a solution of 1-methylethyl 2-(4-{[4-({ethyl[(2-fluorophenyl)methyl]amino}methyl)phenyl]methyl}-1-piperazinyl)-3-pyridinecarboxylate (0.0342 g, 0.068 mmol) in diethyl ether (1 mL)was added 2M HCl in diethylether (0.068 mL, 0.136 mmol) and stirred at room temperature for 1 hour. The solvent was evaporated under nitrogen stream. The solid was dissolved in 1 ml water and lyophilized to give the title compound (35 mg, 86%). LC-MS m/z=505 (M+H), 0.68 minutes (retention time).

### Example 520

### 1-methylethyl 2-{4-[(4-{[ethyl(phenylmethyl)amino]methyl}phenyl)methyl]-1-piperazinyl}-3-pyridinecarboxylate dihydrochloride

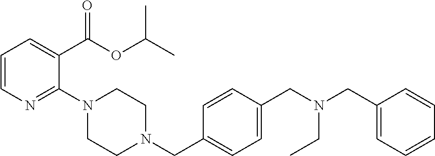 [SMILES: CCN(Cc1ccccc1)Cc1ccc(CN2CCN(c3ncccc3C(=O)OC(C)C)CC2)cc1]

To a solution of 1-methylethyl 2-{4-[(4-{[ethyl(phenylmethyl)amino]methyl}phenyl)methyl]-1-piperazinyl}-3-pyridinecarboxylate (0.0363 g, 0.075 mmol) in diethyl ether (1 mL)was added 2M HCl in diethylether (0.075 mL, 0.149 mmol) and stirred at room temperature for 1 hour. The solvent was evaporated under a nitrogen stream. The solid was dissolved in 1 ml water and lyophilized to give the title compound (37 mg, 86%). LC-MS m/z=486 (M+H), 0.68 minutes (retention time).

### Example 521

### 1-methylethyl2-(4-{[4-({[(2-chloro-6-fluorophenyl)carbonyl]amino}methyl)phenyl]methyl}-1-piperazinyl)-3-pyridinecarboxylate

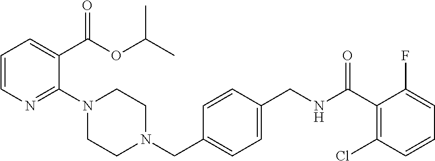 [SMILES: CC(C)OC(=O)c1cccnc1N1CCN(Cc2ccc(CNC(=O)c3c(F)cccc3Cl)cc2)CC1]

To a solution of 1-methylethyl 2-(4-{[4-(aminomethyl)phenyl]methyl}-1-piperazinyl)-3-pyridinecarboxylate (0.058 g, 0.157 mmol) in N,N-Dimethylformamide (DMF) (5 mL) was added 2-chloro-6-fluorobenzoic acid (0.033 g, 0.189 mmol) and HATU (0.072 g, 0.189 mmol) followed by DIEA (0.041 mL, 0.236 mmol). The reaction was stirred at room temperature for 3 hours. The solvent was concentrated. The resulting mixture was purified by Gilson HPLC (Xbridge 30×150 mm 5u preparatory column), eluting at 40 mL/min with a linear gradient running from 50% to 70% with acetonitrile and 0.1% aqueous NH₄OH over 10 minutes to give the freebase of the title compound. (7 mg, 8%). LC-MS m/z=526 (M+H), 0.86 minutes (retention time).

### Example 522

### 1-methylethyl 2-{(3R)-3-[[(3-{[[(2-chloro-6-fluorophenyl)methyl](ethyl)amino]methyl}phenyl)methyl](ethyl)amino]-1-pyrrolidinyl}-3-pyridinecarboxylate

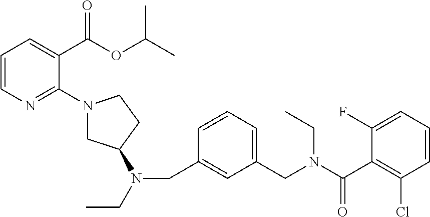 [SMILES: CCN(Cc1cccc(CN(CC)[C@@H]2CCN(c3ncccc3C(=O)OC(C)C)C2)c1)C(=O)c1c(F)cccc1Cl]

To a solution of 1-methylethyl 2-((3R)-3-{ethyl[(3-formylphenyl)methyl]amino}-1-pyrrolidinyl)-3-pyridinecarboxylate (0.100 g, 0.253 mmol) and [(2-chloro-6-fluorophenyl)methyl]ethylamine (0.057 g, 0.303 mmol) in 1,2-dichloroethane (DCE) (3.00 ml) was added acetic acid (0.022 ml, 0.379 mmol) and stirred for 30 minutes. Then sodium triacetoxyborohydride (0.080 g, 0.379 mmol) was added and stirred at room temperature for 4 hours. The reaction was quenched with 1 ml water and the solvents were concentrated. The resulting mixture was purified by Gilson HPLC (Xbridge 30×150 mm 5u preparatory column), eluting at 40 mL/min with a linear gradient running from 80% to 100% with acetonitrile and 0.1% aqueous NH₄OH over 10 minutes to give the freebase of the title compound. (91 mg, 63%). LC-MS m/z=568 (M+H), 0.68 minutes (retention time).

### Example 523

### 1-methylethyl2-(4-{[4-({ethyl[(6-methyl-2-pyridinyl)methyl]amino}methyl)phenyl]methyl}-1-piperazinyl)-3-pyridinecarboxylate quaternary hydrochloride

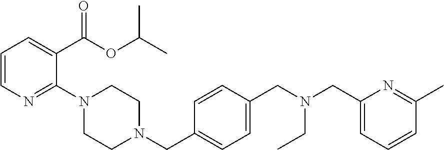 [SMILES: CCN(Cc1ccc(CN2CCN(c3ncccc3C(=O)OC(C)C)CC2)cc1)Cc1cccc(C)n1]

To a solution of 1-methylethyl 2-(4-{[4-({ethyl[(6-methyl-2-pyridinyl)methyl]amino}methyl)phenyl]methyl}-1-piperazinyl)-3-pyridinecarboxylate (0.055 g, 0.110 mmol) in Diethyl ether (1 mL) was added 2M HCl in diethyl ether (0.164 mL, 0.329 mmol) and stirred at room temperature for 1 hour. The solvent was evaporated under nitrogen stream. The solid was dissolved in 1.5 ml water and lyophilized to give the title compound (68 mg, 86%). LC-MS m/z=502 (M+H), 0.69 minutes (retention time).

### Example 524

### 1-methylethyl 2-(4-{[4-({[(2-fluorophenyl)carbonyl]amino}methyl)phenyl]methyl}-1-piperazinyl)-3-pyridinecarboxylate

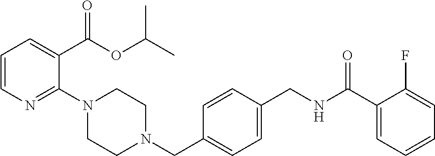 [SMILES: CC(C)OC(=O)c1cccnc1N1CCN(Cc2ccc(CNC(=O)c3ccccc3F)cc2)CC1]

To a solution of 1-methylethyl 2-(4-{[4-(aminomethyl)phenyl]methyl}-1-piperazinyl)-3-pyridinecarboxylate (0.100 g, 0.271 mmol) in N,N-Dimethylformamide (DMF) (5 mL) was added 2-fluorobenzoic acid (0.038 g, 0.271 mmol) and HATU (0.124 g, 0.326 mmol) and then added DIEA (0.071 mL, 0.407 mmol) and stirred at room temperature for 21 hours. The solvent was concentrated and the resulting mixture was purified by Gilson HPLC (Xbridge 30×150 mm 5u preparatory column), eluting at 40 mL/min with a linear gradient running from 60% to 80% with acetonitrile and 0.1% aqueous NH₄OH over 10 minutes to give the freebase of the title compound. (73 mg, 54%). LC-MS m/z=491 (M+H), 0.80 minutes (retention time).

### Example 525

### 1-methylethyl 2-{4-[(4-{[(phenylcarbonyl)amino]methyl}phenyl)methyl]-1-piperazinyl}-3-pyridinecarboxylate

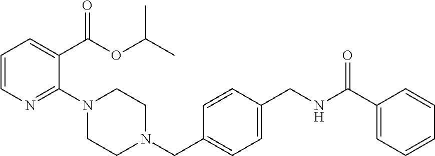 [SMILES: CC(C)OC(=O)c1cccnc1N1CCN(Cc2ccc(CNC(=O)c3ccccc3)cc2)CC1]

To a solution of 1-methylethyl 2-(4-{[4-(aminomethyl)phenyl]methyl}-1-piperazinyl)-3-pyridinecarboxylate (0.100 g, 0.271 mmol) in N,N-Dimethylformamide (DMF) (5 mL) was added benzoic acid (0.033 g, 0.271 mmol) and HATU (0.124 g, 0.326 mmol) and then added DIEA (0.071 mL, 0.407 mmol) and stirred at room temperature for 21 hours. The solvent was concentrated and the resulting mixture was purified by Gilson HPLC (Xbridge 30×150 mm 5u preparatory column), eluting at 40 mL/min with a linear gradient running from 60% to 80% with acetonitrile and 0.1% aqueous NH₄OH over 10 minutes to give the freebase of the title compound. (73 mg, 54%). LC-MS m/z=473 (M+H), 0.80 minutes (retention time).

### Example 526

### 1-methylethyl2-(4-{[4-({[(2-chloro-6-fluorophenyl)methyl]oxy}methyl)phenyl]methyl}-1-piperazinyl)-3-pyridinecarboxylate

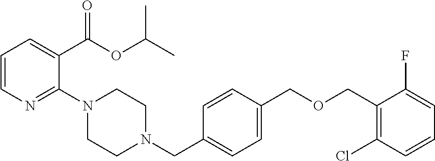 [SMILES: CC(C)OC(=O)c1cccnc1N1CCN(Cc2ccc(COCc3c(F)cccc3Cl)cc2)CC1]

1-methylethyl2-(4-{[4-(hydroxymethyl)phenyl]methyl}-1-piperazinyl)-3-pyridine carboxylate (0.050 g, 0.135 mmol) was dissolved in tetrahydrofuran (THF) (3 mL) and cooled in ice bath. Sodium hydride (3.42 mg, 0.135 mmol) was added and stirred for 15 minutes. Then 2-(bromomethyl)-1-chloro-3-fluorobenzene (0.039 g, 0.175 mmol) in tetrahydrofuran (THF) (1.5 mL) was added and the reaction was stirred at room temperature for 21 hours. LCMS analysis indicated presence of starting material alcohol. Reaction was cooled in ice bath and additional sodium hydride (6.0 mg, 0.250 mmol) was added to reaction and stirred at room temperature for 2 hours. LCMS analysis indicated small decrease in amount of starting material alcohol. 2-(bromomethyl)-1-chloro-3-fluorobenzene (0.015 g, 0.067 mmol) was then added and stirred at room temperature for 2 hours and at 50° C. for 1 hour. LCMS analysis indicated no change in reaction. The reaction was cooled and quenched with 1 ml water. The solvents were concentrated and the resulting mixture was purified by Gilson HPLC (Xbridge 19×150 mm 5u preparatory column), eluting at 18 mL/min with a linear gradient running from 60% to 90% with acetonitrile and 0.1% aqueous NH₄OH over 10 minutes to give the freebase of the title compound. (21 mg, 30%). LC-MS m/z=512 (M+H), 1.00 minutes (retention time).

### Example 527

### 1-methylethyl2-(4-{[4-({[(2-chloro-6-fluorophenyl)methyl]amino}methyl)phenyl]methyl}-1-piperazinyl)-3-pyridinecarboxylate tri hydrochloride

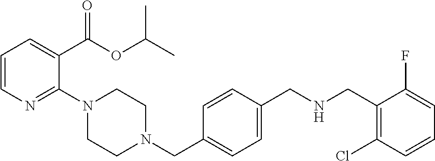 [SMILES: CC(C)OC(=O)c1cccnc1N1CCN(Cc2ccc(CNCc3c(F)cccc3Cl)cc2)CC1]

To a solution of 1-methylethyl 2-(4-{[4-({[(2-chloro-6-fluorophenyl)methyl]amino}methyl)phenyl]methyl}-1-piperazinyl)-3-pyridinecarboxylate (89.36 mg, 0.175 mmol) in Diethyl ether (2 mL) was added 2M HCl (0.262 mL, 0.525 mmol) and stirred at room temperature for 1 hour. The solvent was evaporated under nitrogen stream. The solid was dissolved in 1.5 ml water and lyophilized to give the title compound (89 mg, 77%). LC-MS m/z=539 (M+H), 0.71 minutes (retention time).

### Example 528

### 1-methylethyl 2-{4-[(4-{[[(2-chloro-6-fluorophenyl)carbonyl](ethyl)amino]methyl}phenyl)methyl]-1-piperazinyl}-3-pyridinecarboxylate dihydrochloride

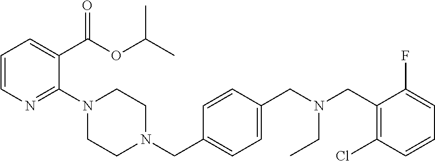 [SMILES: CCN(Cc1ccc(CN2CCN(c3ncccc3C(=O)OC(C)C)CC2)cc1)Cc1c(F)cccc1Cl]

To a solution of 1-methylethyl 2-{4-[(4-{[[(2-chloro-6-fluorophenyl) carbonyl] (ethyl)amino]methyl}phenyl)methyl]-1-piperazinyl}-3-pyridinecarboxylate (69.96 mg, 0.126 mmol) in diethyl ether (2 mL) was added 2M HCl (0.126 mL, 0.253 mmol) and stirred at room temperature for 1 hour. The solvent was evaporated under nitrogen stream. The solid was dissolved in 1.5 ml water and lyophilized to give the title compound which was an inseparable mixture of cis and trans amide isomers. (89 mg, 77%). Isomer A was 33% of the mixture and had LC-MS m/z=554 (M+H), 0.96 minutes (retention time). Isomer B was 67% of the mixture and had LC-MS m/z=554 (M+H), 0.99 minutes (retention time).

Dimer Compounds and Corresponding Precursors/Intermediates

### Example 529

### Bis(1-methylethyl) 2,2′-{benzene-1,4-diylbis[methanediyl(ethylimino)(3R)-3,1-pyrrolidinediyl]}di(3-pyridinecarboxylate)

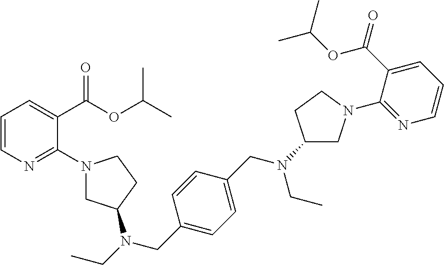 [SMILES: CCN(Cc1ccc(CN(CC)[C@@H]2CCN(c3ncccc3C(=O)OC(C)C)C2)cc1)[C@@H]1CCN(c2ncccc2C(=O)OC(C)C)C1]

To a solution of (R)-isopropyl 2-{3-[tert-butoxycarbonyl(ethyl)amino]pyrrolidin-1-yl}nicotinate (500 mg, 1.32 mmol) in EtOAc (10 mL) was introduced HCl at 0° and the mixture was stirred at room temperature for 30 min. Solvent was evaporated to dryness. The residue was suspended in acetone (20 mL) and to this mixture was added K₂CO₃ (729 mg, 5.28 mmol) followed by α,α′-dibromo-p-xylene (174 mg, 0.66 mmol). The resulting mixture was stirred at reflux for 48 h, monitored via TLC and LCMS. The cooled reaction mixture was filtered; the solid cake was washed with acetone (5 mL). The filtrate was concentrated to dryness which was purified by column chromatography (silica gel, 200-300 um, 50 g) eluting with 500 mL 25% EtOAc of Hexane to give the title compound (325 mg, 38%) as yellow oil. LC-MS m/z 657.4 (M+H)⁺, 2.33 min (ret time); ¹H NMR (400 MHz, CDCl₃) δ 1.01 (t, J=6.8 Hz, 6H), 1.33-1.37 (m, 12H), 1.85-1.98 (m, 2H), 2.05-2.12 (m, 2H), 2.63 (q, J=7.2, 14.4 Hz, 2H), 3.38-3.67 (m, 14H), 5.16-5.19 (m, 2H), 6.58-6.61 (m, 2H), 7.27 (s, 4H), 7.81-7.83 (m, 2H), 8.23-8.25 (m, 2H).

### Example 530

Bis(1-methylethyl) 2,2′-{benzene-1,3-diylbis[methanediyl(ethylimino)(3R)-3,1-pyrrolidinediyl]}i(3-pyridinecarboxylate)

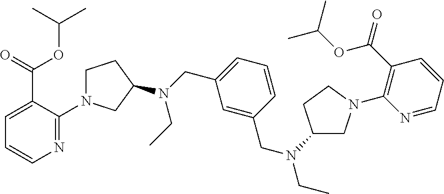 [SMILES: CCN(Cc1cccc(CN(CC)[C@@H]2CCN(c3ncccc3C(=O)OC(C)C)C2)c1)[C@@H]1CCN(c2ncccc2C(=O)OC(C)C)C1]

Following the general procedure of Bis(1-methylethyl) 2,2′-{benzene-1,4-diylbis[methanediyl(ethylimino)(3R)-3,1-pyrrolidinediyl]}di(3-pyridinecarboxylate) (R)-Isopropyl 2-{3-[tert-butoxycarbonyl(ethyl)amino]pyrrolidin-1-yl}nicotinate (100 mg, 0.265 mmol), K₂CO₃ (146 mg, 1.06 mmol) and α,α′-dibromo-m-xylene (35 mg, 0.133 mmol) were reacted to give the title compound (30 mg, 18%) as yellow oil. LC-MS m/z 657.5 (M+H)⁺, 2.31 min (ret time); ¹H NMR (400 MHz, CDCl₃) δ 0.99 (t, J=6.8 Hz, 6H) 1.33-1.36 (m, 12H) 1.88-1.95 (m, 2H) 2.05-2.10 (m, 2H) 2.63 (q, J=7.2, 14.4 Hz, 2H) 3.37-3.69 (m, 14H) 5.16-5.19 (m, 2H) 6.58-6.61 (m, 2H), 7.23-7.30 (m, 4H) 7.81-7.83 (m, 2H) 8.23-8.25 (m, 2H).

### Example 531

### 1-Methylethyl 2-[(3R)-3-(ethyl{[4-({ethyl[(3S)-1-(3-{[(1-methylethyl)oxy]carbonyl}-2-pyridinyl)-3-pyrrolidinyl]amino}methyl)phenyl]methyl}amino)-1-pyrrolidinyl]-3-pyridinecarboxylate

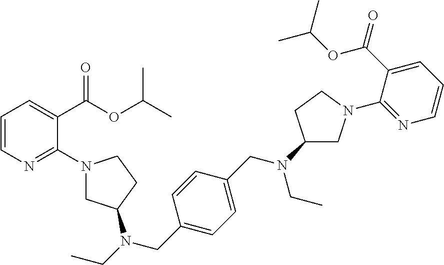 [SMILES: CCN(Cc1ccc(CN(CC)[C@H]2CCN(c3ncccc3C(=O)OC(C)C)C2)cc1)[C@@H]1CCN(c2ncccc2C(=O)OC(C)C)C1]

Following the general procedure of Bis(1-methylethyl) 2,2′-{benzene-1,4-diylbis[methanediyl(ethylimino)(3R)-3,1-pyrrolidinediyl]}di(3-pyridinecarboxylate), (S)-Isopropyl 2-{3-[tert-butoxycarbonyl(ethyl)amino]pyrrolidin-1-yl}nicotinate (50 mg, 0.132 mmol), K₂CO₃ (31 mg, 0.22 mmol) and (R)-isopropyl 2-(3-{[4-(bromomethyl)benzyl](ethyl)amino}pyrrolidin-1-yl)nicotinate (50 mg, 0.11 mmol) were reacted to give the title compound (26 mg, 36%) as yellow oil. LC-MS m/z 657.4 (M+H)⁺, 2.34 min (ret time); ¹H NMR (400 MHz, CDCl₃) δ 1.02 (t, J=6.8 Hz, 6H) 1.33-1.37 (m, 12H) 1.85-1.98 (m, 2H) 2.05-2.12 (m, 2H) 2.63 (q, J=7.2, 14.4 Hz, 2H) 3.38-3.67 (m, 14H) 5.16-5.19 (m, 2H), 6.58-6.61 (m, 2H) 7.27 (s, 4H) 7.81-7.83 (m, 2H) 8.23-8.25 (m, 2H).

### Example 532

### Bis(1-methylethyl) 2,2′-{benzene-1,3-diylbis[methanediyl(2S)-1,2-pyrrolidinediyl methanediyloxy]}di(3-pyridinecarboxylate)

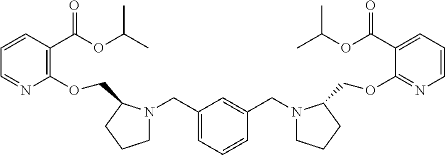 [SMILES: CC(C)OC(=O)c1cccnc1OC[C@@H]1CCCN1Cc1cccc(CN2CCC[C@H]2COc2ncccc2C(=O)OC(C)C)c1]

To a solution of 1-Methylethyl 2-[(3S)-3-amino-1-pyrrolidinyl]-3-pyridinecarboxylate (264 mg, 1 mmol) in acetone (5 mL) was added 1,3-bis(bromomethyl)benzene (132 mg, 0.5 mmol) and K₂CO₃ (207 mg, 1.5 mmol). It was heated at 60° C. for 2 h. The reaction mixture was filtered and the filtrate was concentrated to obtain the crude product. It was purified to obtain the title compound (50 mg, 16%) as colorless oil. LC-MS m/z 631 (M+H)⁺, 1.09 min (ret time); ¹H NMR (400 MHz, CDCl₃) δ 8.26-8.24 (m, 2H), 8.10-8.07 (m, 2H), 7.24-7.18 (m, 4H), 6.91-6.88 (m, 2H), 5.26-5.16 (m, 2H), 4.53-4.49 (m, 2H), 4.30-4.26 (m, 2H), 4.23-4.19 (d, J=12.8 Hz, 2H), 3.43-3.40 (d, J=12.8 Hz, 2H), 3.05-2.98 (m, 2H), 2.93-2.89 (m, 2H), 2.27-2.21 (m, 2H), 2.08-1.98 (m, 2H), 1.84-1.66 (m, 6H), 1.33-1.31 (dd, J=1.2 Hz, 1.6, 12 H).

### Example 533

### Benzene-1,4-diylbis[methanediyl(ethylimino)(3R)-3,1-pyrrolidinediyl-2,3-pyridinediyl methanediyl]bis(3,3-dimethylbutanoate) hydrochloride

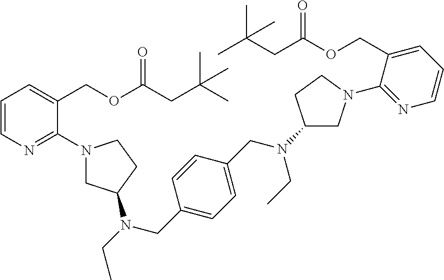 [SMILES: CCN(Cc1ccc(CN(CC)[C@@H]2CCN(c3ncccc3COC(=O)CC(C)(C)C)C2)cc1)[C@@H]1CCN(c2ncccc2COC(=O)CC(C)(C)C)C1]

A mixture of (R)-(2-(3-(ethylamino)pyrrolidin-1-yl)pyridin-3-yl)methyl 3,3-dimethylbutanoate (426 mg, 1.3 mmol) and 1,4-bis(bromomethyl)benzene (176 mg, 0.7 mmol) in acetone (10 mL) was heated to 60° C. K₂CO₃ (184 mg, 1.3 mmol) was added. It was heated at reflux for 2 h. The reaction mixture was filtered. The filtrate was concentrated to obtain the crude product.

Another batch of material was prepared with same reactants and reagents under similar reaction conditions as above, the product of which, was combined with crude product as identified above. The combined crude material was purified by silica gel column eluting with a mixture of 10% ethyl acetate, 4% of Et₃N in petroleum ether to give the free base of the title compound (300 mg, 36%) as yellow oil. It was dissolved in 5 mL of ether; the solution of HCl in ether (2 mL, 1 mol/L) was added. It was stirred at room temperature for 10 min. Solvent was removed to give the title compound (310 mg, 99% combined yield of two (2) batches of product) as white solid. LC-MS m/z 741.4 (M+H)⁺, 1.28 min (ret time); ¹H NMR (400 MHz, CDCl₃) δ 1.33 (s, 6H) 3.01-3.64 (m, 14H) 5.07-5.15 (m, 4H) 6.76-6.79 (m, 2H) 7.57-7.73 (m, 6H) 8.15-8.16 (m, 2H) 12.84 (s, 1H)

### Example 534

### Benzene-1,4-diylbis[methanediyl(ethylimino)(3R)-3,1-pyrrolidinediyl-2,3-pyridinediylmethanediyl]dibenzoate hydrochloride

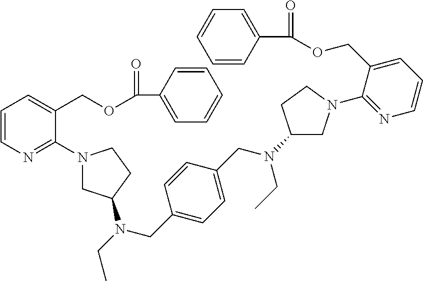 [SMILES: CCN(Cc1ccc(CN(CC)[C@@H]2CCN(c3ncccc3COC(=O)c3ccccc3)C2)cc1)[C@@H]1CCN(c2ncccc2COC(=O)c2ccccc2)C1]

A mixture of (R)-(2-(3-(ethylamino)pyrrolidin-1-yl)pyridin-3-yl)methyl benzoate (630 mg, 1.9 mmol) and 1,4-bis(bromomethyl)benzene (256 mg, 0.97 mmol) in acetone (10 mL) was heated to 60° C. K₂CO₃ (401 mg, 2.9 mmol) was added. It was heated at reflux for 2 h. The reaction mixture was filtered. The filtrate was concentrated to obtain the crude product. It was purified by silica gel column eluting with a mixture of 14% of ethyl acetate and 5% of Et₃N in petroleum ether to give the free base of the title compound (280 mg, 38%) as yellow oil. It was dissolved in ether (10 mL); the solution of HCl in ether (5 mL, 1 mol/L) was added and stirred at room temperature for 30 min. Solvent was removed to give the title compound (290 mg, 99%) as yellow solid. LC-MS m/z 753.3 (M+H)⁺, 1.34 min (ret time); ¹H NMR (400 MHz, CDCl₃) δ 1.19-1.25 (m, 6H) 2.59-3.18 (m, 8H) 4.15-4.49 (m, 14H) 5.47 (s, 4H) 6.83 (s, 2H) 7.44-8.23 (m, 18H) 12.73 (s, 1H)

### Example 535

### Bis(1-methylethyl) 2,2′-[benzene-1,4-diylbis(methanediyl-4,1-piperazinediyl)]di(3-pyridinecarboxylate)

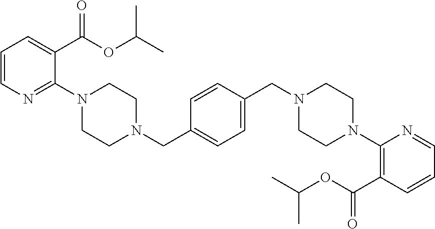 [SMILES: CC(C)OC(=O)c1cccnc1N1CCN(Cc2ccc(CN3CCN(c4ncccc4C(=O)OC(C)C)CC3)cc2)CC1]

To a solution of 1-methylethyl 2-{4-[(4-formylphenyl)methyl]-1-piperazinyl}-3-pyridinecarboxylate (175 mg, 0.476 mmol) in dimethyl sulfoxide (DMSO) (2 mL), AcOH (0.055 mL, 0.953 mmol), 1-methylethyl 2-(1-piperazinyl)-3-pyridinecarboxylate (178 mg, 0.714 mmol) and sodium triacetoxyborohydride (303 mg, 1.429 mmol) were added. After the reaction mixture was stirred at R.T. for 16 hrs, it was purified by Gilson-HPLC in basic condition (Acetonitrile: water+0.1% NH4OH, Gradient: 50% to 80% B at 15 min) to get title compound as white solid (90 mg, 29.9%). LC-MS m/z 601.4 (M+H)⁺, 0.83 min (ret time).

### Example 536

### Bis(1-methylethyl) 2,2′-{benzene-1,4-diylbis[methanediyl(2S)-1,2-pyrrolidinediyl methanediyloxy]}di(3-pyridinecarboxylate)

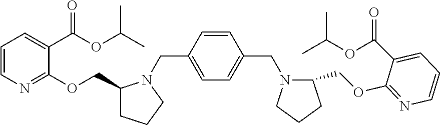 [SMILES: CC(C)OC(=O)c1cccnc1OC[C@@H]1CCCN1Cc1ccc(CN2CCC[C@H]2COc2ncccc2C(=O)OC(C)C)cc1]

To a solution of 1-Methyl ethyl 2-{[(2R)-2-pyrrolidinylmethyl]oxy}-3-pyridinecarboxylate (264 mg, 1 mmol) in acetone (5 mL) was added 1,4-bis(bromomethyl)benzene (132 mg, 0.5 mmol) and K₂CO₃ (207 mg, 1.5 mmol). It was heated at 60° C. for 2 h. The reaction mixture was filtered and the filtrate was concentrated to obtain the crude product. It was purified to obtain the title compound (50 mg, 16%) as yellow oil. LC-MS m/z 631 (M+H)⁺, 1.01 min (ret time); ¹H NMR (400 MHz, CDCl₃) δ 8.26-8.25 (m, 2 H), 8.10-8.09 (m, 2H), 7.28-7.23 (m, 4H), 6.94-6.89 (m, 2H), 5.26-5.18 (m, 2H), 4.53-4.49 (m, 2H), 4.31-4.27 (m, 2H), 4.24-4.21 (m, J=12.8 Hz, 2H), 3.42-3.39 (d, J=12.8 Hz, 2H), 3.03-3.02 (m, 2H), 2.91 (m, 2H), 2.24-2.21 (m, 2H), 2.04-2.03 (m, 2 H), 1.79-1.71 (m, 6H), 1.33-1.32 (d, J=4.8 Hz, 12H).

### Example 537

### Bis(1-methylethyl) 2,2′-{benzene-1,4-diylbis[methanediyl(ethylimino)(3S)-3,1-pyrrolidinediyl]}di(3-pyridinecarboxylate)

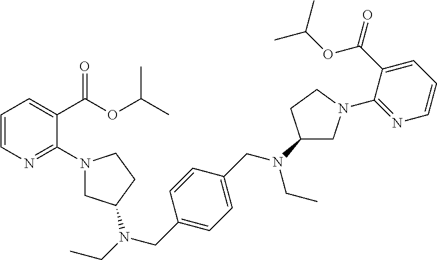 [SMILES: CCN(Cc1ccc(CN(CC)[C@H]2CCN(c3ncccc3C(=O)OC(C)C)C2)cc1)[C@H]1CCN(c2ncccc2C(=O)OC(C)C)C1]

1,4-bis(bromomethyl)benzene (2.53 g, 9.58 mmol) was added to a suspension of 1-methylethyl 2-[(35)-3-(ethylamino)-1-pyrrolidinyl]-3-pyridinecarboxylate (7.8 g, 19.93 mmol) and potassium carbonate (8.29 g, 60.0 mmol) in acetonitrile (75 mL) at ambient temperature. The resulting suspension was allowed to stir. After 17 hrs an additional 0.3 g of amine starting material was added. After ˜22 hrs the reaction suspension was filtered, washed with ethyl acetate and the filtrate concentrated to give 8.63 g of tan gum. This was taken into ethyl acetate and extracted with water (2×). The organic phase was then extracted with HCl solution (pH 1-2) (4×). The organic phase was extracted with brine (1×), dried over magnesium sulfate, filtered, and concentrated to give 5.95 g of a clear, light brown liquid. This was passed through a plug of silica gel using a 2 L fritted funnel filled ˜½ with silica gel. A solvent gradient consisting of 5%, 10%, 20%, 30%, 40%, 50%, and 100% ethyl acetate/hexanes was used to elute the product and gave 4.0 g of clear, colorless oil (isolate A).

The combined acidic aqueous phase was basified with 6N NaOH and extracted with ethyl acetate. The organic phase was dried over magnesium sulfate, filtered and concentrated to give 0.73 g of a clear, light tan oil. This was passed through a plug of silica gel using a 1 L fritted funnel filled ˜½ with silica gel. 100% DCM was used as eluent followed by 100% ethyl acetate to give 0.71 g of a clear, colorless oil (isolate B).

The combined isolates A and B were subjected to purification via Gilson HPLC purification method under the following conditions: Column: XBridge 30×150 mm 5u, Mobile phase: Acetonitrile: Water+0.1% NH₄OH, Flow rate: 40 ml/min, Gradient: 80%-100% B for 10 min. to give 3.672 g of clear, oil. This was taken into 10 mL of methanol afterwhich time 0.711 g of L-tartaric acid was added and the suspension stirred to complete dissolution. The resulting solution was concentrated to a gel. The gel was re-dissolved in methanol and ether added. The solution was concentrated and pumped to a white solid. This was taken into ˜80 mL of water and lyophilized to give 3.14 g of a white solid. LC/MS m/z-657.8 (M+H); ¹H NMR (400 MHz, MeOD₄) δ 1.18 (t, J=8 Hz, 6H), 1.38 (t, J=8 Hz, 12H), 2.01-2.12 (m, 2H), 2.3-2.4 (m, 2H), 2.87 (q, J=8 Hz, 4H), 3.50-3.68 (m, 10H), 3.90-4.05 (m, 2H), 4.45 (s, 2H), 5.15-5.21 (m, 2H), 6.72-6.65 (m, 2H), 7.45 (s, 4H), 7.90-7.92 (m, 2H), 8.21-8.22 (m, 2H).

### Example 42

### 1-Methylethyl 2-[(3R)-3-(ethyl{[4-({ethyl[(3R)-1-(2-methylpropanoyl)-3-pyrrolidinyl]amino}methyl)phenyl]methyl}amino)-1-pyrrolidinyl]-3-pyridinecarboxylate (recited supra, reiterated here)

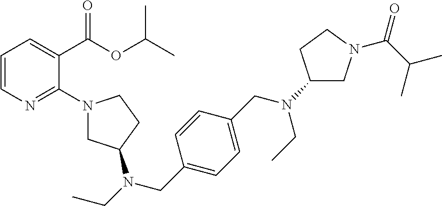 [SMILES: CCN(Cc1ccc(CN(CC)[C@@H]2CCN(c3ncccc3C(=O)OC(C)C)C2)cc1)[C@@H]1CCN(C(=O)C(C)C)C1]

To a solution of 1-methylethyl 2-[(3R)-3-(ethylamino)-1-pyrrolidinyl]-3-pyridinecarboxylate (85 mg, 0.385 mmol) and potassium carbonate (160 mg, 1.155 mmol) in acetone (10 mL) at room temperature was added (3R)—N-{[4-(bromomethyl)phenyl]methyl}-N-ethyl-1-(2-methylpropanoyl)-3-pyrrolidinamine (230 mg, 0.385 mmol) in one portion. The resulting mixture was heated to reflux for 24 h. It was cooled to room temperature. The reaction mixture was filtered and the filtrate was concentrated under reduced pressure to give crude product. It was purified by Pre-TLC eluting with EtOAc to give the title compound (26 mg, 11%) as pale yellow solid. LC-MS m/z 564.4 (M+H)⁺, 2.21 min (ret time); ¹H NMR (400 MHz, CDCl₃) δ 8.26-8.24 (m, 1H), 7.84-7.82 (m, 1H), 7.83-7.28 (m, 4H), 6.65-6,7-(m, 1H), 5.20-5.17 (m, 1H), 3.70-3.28 (m, 14H), 2.66-2.59 (m, 5H), 2.10-1.90 (m, 4H), 1.38-1.34 (m, 6H), 1.14-0.99 (m, 12H).

### Example 538

### Bis(1-methylethyl)2,2′-[(ethylimino)bis(methanediylbenzene-4,1-diylmethanediyl-4,1-piperazinediyl)]di(3-pyridinecarboxylate)

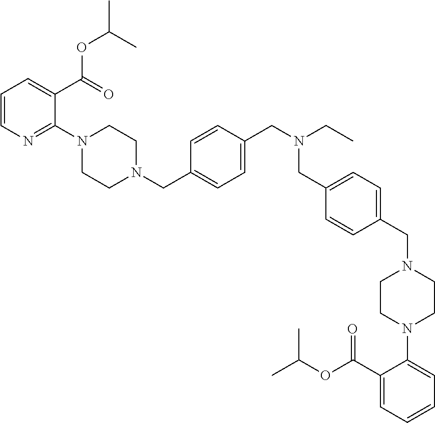 [SMILES: CCN(Cc1ccc(CN2CCN(c3ccccc3C(=O)OC(C)C)CC2)cc1)Cc1ccc(CN2CCN(c3ncccc3C(=O)OC(C)C)CC2)cc1]

1-Methylethyl 2-{-4-[(4-formylphenyl)methyl]-1-piperazinyl}-3-pyridinecarboxylate (1.06 g, 2.88 mmol), and methanol (11.37 ml) were combined in a 20 mL vial and then 2M in THF ethylamine (2.88 ml, 5.77 mmol) and acetic acid (0.165 ml, 2.88 mmol) were added at 23° C. Stirred 5 min and then sodium cyanoborohydride (0.635 g, 10.10 mmol) was added. The resulting mixture was stirred in the sealed vial at 23° C. for 16 h. The reaction was diluted with EtOAc (˜150 mL) and washed with 1M aq NaOH (50 mL), water (2×50 mL) and satd aq NaCl (50 mL) and concentrated to afford an oil. The crude product was purified on a silica cartridge (40 g) with a Combiflash Companion, eluting at 40 mL/min with a gradient running from dichloromethane to 100% of a 10% solution of 2M NH₃ in MeOH in dichloromethane over 30 min. Two products were isolated. The first compound to elute was the dimeric title compound. The second compound to elute was the free base of the compound of the previous experiment.

The dimer was further purified by preparative hplc (The crude product was dissolved in DMSO (1 mL), filtered through a 0.45 μm acrodisc syringe filter, and purified on a Gilson HPLC (XBridge C18 30×150 mm 5p preparatory column), eluting at 40 mL/min with a linear gradient running from 50% CH₃CN in H₂O (0.1% NH₄OH) to 100% CH₃CN over 20 min.) The desired fractions were concentrated under a stream of nitrogen at 50° C. to afford the title compound (82 mg, 3.8%) was obtained as a partially purified oil. LC-MS m/z 748.1 (M+H)⁺ 0.80 (ret time).

### Example 539

### (3R)—N,N-diethyl-N-{[4-({ethyl[(3R)-1-(3-{[(1-methylethyl)oxy]carbonyl}-2-pyridinyl)-3-pyrrolidinyl]amino}methyl)phenyl]methyl}-1-(3-{[(1-methylethyl)oxy]carbonyl}-2-pyridinyl)-3-pyrrolidinaminium

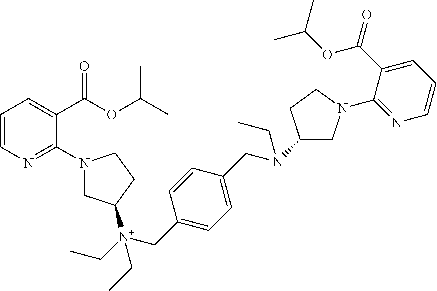 [SMILES: CCN(Cc1ccc(C[N+](CC)(CC)[C@@H]2CCN(c3ncccc3C(=O)OC(C)C)C2)cc1)[C@@H]1CCN(c2ncccc2C(=O)OC(C)C)C1]

bis(1-Methylethyl) 2,2′-{benzene-1,4-diylbis[methanediyl(ethylimino)(3R)-3,1-pyrrolidinediyl]}di(3-pyridinecarboxylate) (97.5 mg, 0.148 mmol) was dissolved in acetonitrile (4 mL) and ethyl iodide (0.20 mL, 2.475 mmol) was added and the solution was stirred in a tightly sealed 20 mL vial for 3 days. Additional ethyl iodide (1 mL) was added. The mixture was stirred for 4 weeks. The volatiles were concentrated under a stream of nitrogen at 50° C. to afford 0.152 g of an amber oil. Purification was by preparative hplc (The crude product was dissolved in DMSO (1 mL), filtered through a 0.45 μm acrodisc syringe filter, and purified on a Gilson HPLC (Atlantis 19×150 mm 5p preparatory column), eluting at 16 mL/min with a linear gradient running from 30% CH₃CN in H₂O to 60% CH₃CN over 12 min.) The desired fractions were concentrated under a stream of nitrogen at 50° C., to afford the title compound as a light beige solid (12 mg, 9.95%). Returned and the spectrum at 100° C. supports the presence of rotational isomers at room temp. The nmr and C13 supports the proposed mono salt structure also indicated by the lcms. LC-MS m/z 685.8 (M)⁺ 0.80 (ret time).

### Example 540

### 1H-pyrazole-3,5-diylbis[methanediyl(ethylimino)(3R)-3,1-pyrrolidinediyl-2,3-pyridinediyl methanediyl]bis(3,3-dimethylbutanoate) quaternary hydrochloride

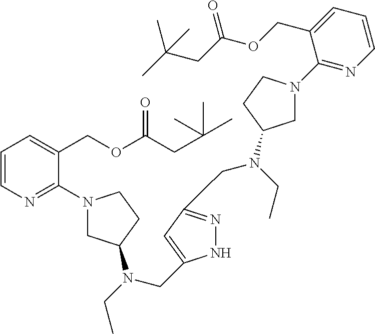 [SMILES: CCN(Cc1cc(CN(CC)[C@@H]2CCN(c3ncccc3COC(=O)CC(C)(C)C)C2)[nH]n1)[C@@H]1CCN(c2ncccc2COC(=O)CC(C)(C)C)C1]

To a solution of 1,1-dimethylethyl 3,5-bis{[[(3R)-1-(3-{[(3,3-dimethylbutanoyl)oxy]methyl}-2-pyridinyl)-3-pyrrolidinyl](ethyl)amino]methyl}-1H-pyrazole-1-carboxylate (0.138 g, 0.166 mmol) in dichloromethane (DCM) (5 mL) was added trifluoroacetic acid (0.250 mL, 3.24 mmol) and stirred at room temperature for 4 hours. The resulting mixture was purified by Gilson HPLC (Sunfire 30×150 mm 5u preparatory column), eluting at 40 mL/min with a linear gradient running from 25% to 45% with acetonitrile and water with 0.1% TFA over 8 minutes to give the TFA salt of the title compound. The product fractions were pooled and diluted with EtOAc. Aqueous layer was neutralized with sat. NaHCO₃ and separated. Aqueous layer was extracted 3× EtOAc. Organic layers were washed with water, brine, dried MgSO₄, and concentrated to give the free base of the title compound (82 mg). The compound was dissolved in ether (1 mL), and 2M HCl in diethyl ether (0.164 ml, 0.328 mmol) (2.9 eq vs free base compound) was added and stirred for 3 hours, concentrated and dried under vacuum pump. The solid was then dissolved in 2 ml water and lyophilized to give the title compound (97 mg, 66%) as white solid. LC-MS m/z=731 (M+H), 0.87 minutes (retention time).

### Example 541

### 2,5-pyrazinediylbis[methanediyl(ethylimino)(3R)-3,1-pyrrolidinediyl-2,3-pyridinediyl methanediyl]bis(3,3-dimethylbutanoate) hydrochloride

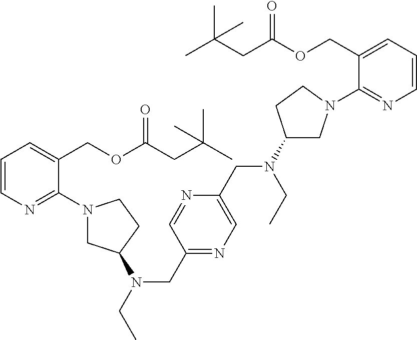 [SMILES: CCN(Cc1cnc(CN(CC)[C@@H]2CCN(c3ncccc3COC(=O)CC(C)(C)C)C2)cn1)[C@@H]1CCN(c2ncccc2COC(=O)CC(C)(C)C)C1]

To a solution of {2-[(3R)-3-(ethylamino)-1-pyrrolidinyl]-3-pyridinyl}methyl 3,3-dimethylbutanoate (0.100 g, 0.255 mmol) and 2,5-bis(bromomethyl)pyrazine (0.120 g, 0.451 mmol) in acetonitrile (3 mL) and water (1 mL) was added potassium iodide (0.042 g, 0.255 mmol) and stirred for 5 minutes. Then potassium carbonate (0.053 g, 0.382 mmol) was added and stirred at room temperature for 22 hours. The solvent was concentrated and the residue was dissolved in EtOAc and washed with water (2×), brine dried MgSO₄ concentrated. The resulting mixture was purified by Gilson HPLC (Sunfire 19×150 mm 5u preparatory column), eluting at 18 mL/min with a linear gradient running from 15% to 45% with acetonitrile and water with 0.1% TFA over 8 minutes to give the TFA salt of the title compound. The title compound was dissolved in water and was neutralized with saturated NaHCO₃ and separated. The aqueous layer was extracted EtOAc (3×). Organic layers were washed with water, brine, dried MgSO₄, concentrated to give the free base of the title compound. The compound was dissolved in diethyl ether (1.50 mL), and added 2M HCl in diethyl ether (0.144 mL, 0.288 mmol) (2 eq with respect to isolated freebase product) was added, stirred at room temperature for 1 hour and concentrated solvent. The solid was then dissolved in 2 ml water and lyophilized to give the title compound (46 mg, 15%). LC-MS m/z=743 (M+H), 0.68 minutes (retention time).

### Example 542

### bis(1-methylethyl)2,2′-{benzene-1,4-diylbis[methanediylimino(3R)-3,1-pyrrolidinediyl]}di(3-pyridinecarboxylate)

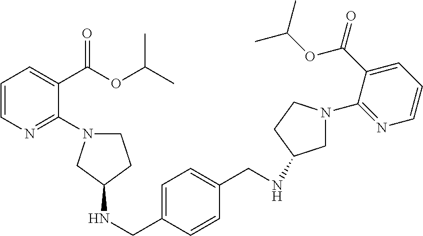 [SMILES: CC(C)OC(=O)c1cccnc1N1CC[C@@H](NCc2ccc(CN[C@@H]3CCN(c4ncccc4C(=O)OC(C)C)C3)cc2)C1]

To a solution of 1-methylethyl 2-[(3R)-3-amino-1-pyrrolidinyl]-3-pyridinecarboxylate hydrochloride (0.150 g, 0.466 mmol) and terephthaldicarboxaldehyde (0.031 g, 0.233 mmol) in 1,2-dichloroethane (DCE) (6.00 ml) was added acetic acid (0.027 ml, 0.466 mmol) and stirred at room temperature for 1 hour. Sodium triacetoxyborohydride (0.197 g, 0.931 mmol) was then added and stirred at room temperature for 3 hours. LCMS analysis indicated that no product had been formed. DIEA (0.244 ml, 1.397 mmol) was then added and stirred for 20 hours. LCMS analysis indicated formation of product. Additional terephthaldicarboxaldehyde (10 mg, 0.075 mmol) and sodium triacetoxyborohydride (0.049 g, 0.233 mmol) was added and stirred for 19 hours. The reaction was quenched with 1 ml water and the solvents were concentrated and the resulting mixture was purified by Gilson HPLC (Xbridge 30×150 mm 5u preparatory column), eluting at 40 mL/min with a linear gradient running from 50% to 70% with acetonitrile and 0.1% aqueous NH₄OH over 10 minutes to give the freebase of the title compound. (19 mg, 6%). LC-MS m/z=601 (M+H), 0.69 minutes (retention time).

### Example 510

### (Reiterated Here—Mixture of Products, Inc. Dlmer bis(1-methylethyl) 2,2′-[2,5-pyrazinediylbis(methanediyl-4,1-piperazinediyl)]di(3-pyridine carboxylate)

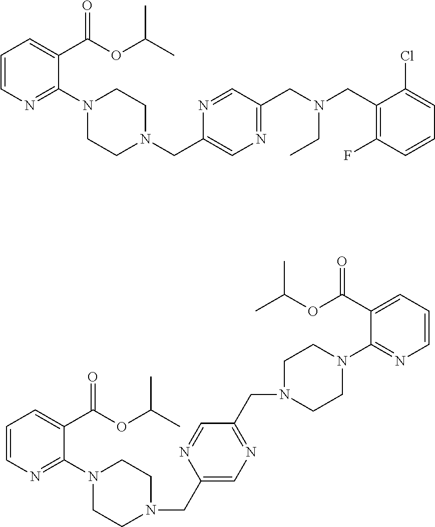 [SMILES: CCN(Cc1cnc(CN2CCN(c3ncccc3C(=O)OC(C)C)CC2)cn1)Cc1c(F)cccc1Cl ; CC(C)OC(=O)c1cccnc1N1CCN(Cc2cnc(CN3CCN(c4ncccc4C(=O)OC(C)C)CC3)cn2)CC1]

To a solution of 1-methylethyl 2-(1-piperazinyl)-3-pyridinecarboxylate (0.050 g, 0.201 mmol), 2,5-bis(bromomethyl)pyrazine (0.053 g, 0.201 mmol) and [(2-chloro-6-fluorophenyl)methyl]ethylamine (0.038 g, 0.201 mmol) in acetone (4 mL) was added K₂CO₃ (0.055 g, 0.401 mmol) and heated to 50° C. for 4 hours and then stirred at room temperature for 18 hours. The reaction was then filtered and solvent concentrated. The resulting mixture was purified by Gilson HPLC (Xbridge 19×150 mm 5u preparatory column), eluting at 18 mL/min with a linear gradient running from 50% to 100% with acetonitrile and 0.1% aqueous NH₄OH over 20 minutes to give the freebase of:
1-methylethyl2-{4-[(5-{[[(2-chloro-6-fluorophenyl)methyl](ethyl)amino]methyl}-2-pyrazinyl)methyl]-1-piperazinyl}-3-pyridinecarboxylate (29 mg, 26%). LC-MS m/z=541 (M+H), 0.69 minutes (retention time).
bis(1-methylethyl)2,2′-[2,5-pyrazinediylbis(methanediyl-4,1-piperazinediyl)]di(3-pyridinecarboxylate) (24 mg, 19%). LC-MS m/z=602 (M+H), 0.74 minutes (retention time).

It is to be understood that the invention is not limited to the embodiments illustrated hereinabove and the right is reserved to the illustrated embodiments and all modifications coming within the scope of the following claims.

The various references to journals, patents, and other publications which are cited herein comprise the state of the art and are incorporated herein by reference as though fully set forth.

What is claimed is:

1-5. (canceled)

6. A compound of Formula (IV): 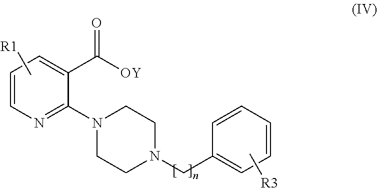
wherein:
n is 0 or an integer from 1 to 5;
Y is straight or branched C₁₋₆ alkyl or cycloalkyl;
R₁ is H, halogen, straight or branched C₁₋₆ alkyl, phenyl, substituted phenyl, —NHR₁ₐ, —SR_{1b} or —OR_{1c};
R₃ is one or more substituents independently selected from —H, —OH, —CN, halogen, straight or branched C₁₋₆ alkyl, -straight or branched C₁₋₆ haloalkyl, -straight or branched C₁₋₆ alkoxy, -straight or branched C₁₋₆ alkoxy, —O(CH₂)ₓOR_{1d}, —C(O)R₁ₑ, —C(O)OR_{1f}, —phenyl, —(CH₂)ₓ-phenyl, —(CH₂)ₓ-substituted phenyl, -phenyloxy, -substituted phenyloxy, —(CH₂)ₓ-phenyloxy, —(CH₂)ₓ-piperazinyl, —(CH₂)ₓ-substituted piperazinyl, —(CH₂)ₓ—N-substituted piperazinyl, —(CH₂)ₓNRC(O)-phenyl, —(CH₂)ₓNRC(O)-substituted phenyl, —O—(CH₂)ₓ-phenyl, —O—(CH₂)ₓ-substituted phenyl, —O(CH₂)ₓ-1,4-benzodioxinyl, —O(CH₂)ₓ-naphthalenyl, —O(CH₂)ₓ-tetrazolyl, —S-phenyl, —S(CH₂)ₓphenyl, —SO₂R_{1g}, —SO₂N(R_{1g})₂, —(CH₂)ₓ—N(R₁ₕ)—(CH₂)ₓR₁ᵢ;
wherein:
R₁ₐ, R_{1b} or R_{1c} as defined in R₁ above is phenyl or substituted phenyl;
R, R_{1d}, R₁ₑ, R_{1f}, R_{1g} or R₁ₕ as defined in R₃ is H, straight or branched C₁₋₆ alkyl;
R₁ᵢ is phenyl, substituted phenyl, furanyl, substituted furanyl, thienyl, or substituted thienyl;
x as defined for substituents defined above is 0 or an integer from 1 to 5;
wherein:
each substitutent as defined in R₃ above further is optionally substituted by one or more of following substituents selected from: —H, —OH, —CN, —NO₂,-halogen, —(CH₂)_{y}—OH, —O(CH₂)_{y} CN, —OC(O)OH, —OC(O)R₁ⱼ, —C(O)OR₁ₖ, —O(CH₂)_{y}OR₁ₗ,-straight or branched C₁₋₆ alkyl,-straight or branched C₁₋₆ haloalkyl, -straight or branched C₁₋₆ straight or branched alkoxy, —NR₁ₘR₁ₙ, —SO₂R₁ₒ, —S(CH₂)_{y}R₁ₚ, —NR_{1q}C(O)R₁ᵣ, aryl or heteroaryl;
wherein:
y as defined for variables above is 0 or an integer from 1 to 5,
R₁ⱼ, R₁ₖ, R₁ᵢ, R₁ₘ, R₁ₙ, R₁ₒ, R₁ₚ, R_{1q} or R₁ᵣ is H, straight or branched C₁₋₆ alkyl, phenyl, substituted phenyl, pyridinyl, or substituted pyridinyl, —C(O)-phenyl, —C(O)substituted phenyl or (CH₂)ₓ-2-oxo-1-pyrrolidinyl or (CH₂)ₓ-2-oxo-N-pyrrolidinyl; or
wherein:
x is 0 or an integer from 1 to 5;
each phenyl or substituted phenyl substitutent as defined in R₁ⱼ, R₁ₖ, R₁ₗ, R₁ₘ, R₁ₙ, R₁ₒ, R₁ₚ, R_{1q} or R₁ᵣ above further is optionally substituted by one or more of following substituents selected from:
—H, —OH, —CN, —NO₂,-halogen, —(CH₂)_{y}—OH,
—OC(O)OH, —OC(O)R₁ₛ, —C(O)OR₁ₜ, —SO₂N(R₁ᵤ)—2-, straight or C₁₋₆ alkyl,-straight or branched C₁₋₆ haloalkyl, -straight or branched C₁₋₆ alkoxy;
wherein:
R₁ₛ, R₁ₜ, or R₁ᵤ as defined above is H, straight or branched
C₁₋₆ alkyl, phenyl or substituted phenyl; or
a pharmaceutically acceptable salt thereof.

7. A compound which is:
1-methylethyl 2-[4-({3-[(2-thienylmethyl)oxy]phenyl}methyl)-1-piperazinyl]-3-pyridinecarboxylate;
1-methylethyl 2-{4-[(3-{[(2,6-dichlorophenyl)methyl]oxy}phenyl)methyl]-1-piperazinyl}-3-pyridinecarboxylate;
1-methylethyl 2-{4-[(3-{[(3-chlorophenyl)methyl]oxy}phenyl)methyl]-1-piperazinyl}-3-pyridinecarboxylate; 1-methylethyl 2-{4-[(3-{[(2-chloro-4-fluorophenyl)methyl]oxy}phenyl)methyl]-1-piperazinyl}-3-pyridinecarboxylate;
1-methylethyl 2-{4-[(3-{[(4-methylphenyl)methyl]oxy}phenyl)methyl]-1-piperazinyl}-3-pyridinecarboxylate;
1-methylethyl 2-{4-[(3-{[(2-methylphenyl)methyl]oxy}phenyl)methyl]-1-piperazinyl}-3-pyridinecarboxylate;
1-methylethyl 2-{4-[(3-{[(3-fluorophenyl)methyl]oxy}phenyl)methyl]-1-piperazinyl}-3-pyridinecarboxylate;
1-methylethyl 2-{4-[(3-{[(4-nitrophenyl)methyl]oxy}phenyl)methyl]-1-piperazinyl}-3-pyridinecarboxylate;
1-methylethyl 2-{4-[(3-{[(2-chlorophenyl)methyl]oxy}phenyl)methyl]-1-piperazinyl}-3-pyridinecarboxylate;
1-methylethyl 2-(4-{[3-({[3-(trifluoromethyl)phenyl]methyl}oxy)phenyl]methyl}-1-piperazinyl)-3-pyridinecarboxylate;
1-methylethyl 2-{4-[(3-{[(2,4-dichlorophenyl)methyl]oxy}phenyl)methyl]-1-piperazinyl}-3-pyridinecarboxylate;
1-methylethyl 2-{4-[(3-{[(3-methylphenyl)methyl]oxy}phenyl)methyl]-1-piperazinyl}-3-pyridinecarboxylate;
1-methylethyl 2-(4-{[3-(ethyloxy)phenyl]methyl}-1-piperazinyl)-3-pyridine carboxylate;
1-methylethyl 2-{4-[(3-{[(2-chloro-6-fluorophenyl)methyl]oxy}phenyl)methyl]-1-piperazinyl}-3-pyridinecarboxylate;
1-methylethyl 2-(4-{[3-(acetyloxy)phenyl]methyl}-1-piperazinyl)-3-pyridine carboxylate;
1-methylethyl 2-[4-({3-[(1,1,2,2-tetrafluoroethyl)oxy]phenyl}methyl)-1-piperazinyl]-3-pyridinecarboxylate;
1-methylethyl 2-[4-({3-[(2-methylpropyl)oxy]phenyl}methyl)-1-piperazinyl]-3-pyridinecarboxylate;
1-methylethyl 2-(4-{[3-(propyloxy)phenyl]methyl}-1-piperazinyl)-3-pyridine carboxylate;
[(3-{[4-(3-{[(1-methylethyl)oxy]carbonyl}-2-pyridinyl)-1-piperazinyl]methyl}phenyl)oxy]acetic acid;
1-methylethyl 2-[4-({3-[(2-hydroxyethyl)oxy]phenyl}methyl)-1-piperazinyl]-3-pyridinecarboxylate;
1-methylethyl 2-[4-({3-[(phenylmethyl)oxy]phenyl}methyl)-1-piperazinyl]-3-pyridinecarboxylate;
1-methylethyl 2-(4-{[3-({2-[(2-chloroethyl)oxy]ethyl}oxy)phenyl]methyl}-1-piperazinyl)-3-pyridinecarboxylate;
1-methylethyl 2-{4-[(3-{[(4-fluorophenyl)methyl]oxy}phenyl)methyl]-1-piperazinyl}-3-pyridinecarboxylate;
1-methylethyl 2-{4-[(3-{[(4-chlorophenyl)methyl]oxy}phenyl)methyl]-1-piperazinyl}-3-pyridinecarboxylate;
1-methylethyl 2-[4-(phenylmethyl)-1-piperazinyl]-4-(phenyloxy)-3-pyridinecarboxylate;
1-methylethyl 4-[(2-fluorophenyl)amino]-2-[4-(phenylmethyl)-1-piperazinyl]-3-pyridinecarboxylate;
1-methylethyl 4-[(3-chlorophenyl)amino]-2-[4-(phenylmethyl)-1-piperazinyl]-3-pyridinecarboxylate;
1-methylethyl 4-[(4-cyanophenyl)amino]-2-[4-(phenylmethyl)-1-piperazinyl]-3-pyridinecarboxylate;
1-methylethyl 4-{[2-(ethyloxy)phenyl]amino}-2-[4-(phenylmethyl)-1-piperazinyl]-3-pyridinecarboxylate;
1-methylethyl 4-{[4-(1-methylethyl)phenyl]amino}-2-[4-(phenylmethyl)-1-piperazinyl]-3-pyridinecarboxylate;
1-methylethyl 4-{[2-(1-methylethyl)phenyl]amino}-2-[4-(phenylmethyl)-1-piperazinyl]-3-pyridinecarboxylate;
1-methylethyl 4-({3-[(ethyloxy)carbonyl]phenyl}amino)-2-[4-(phenylmethyl)-1-piperazinyl]-3-pyridinecarboxylate;
1-methylethyl 4-[(2-ethylphenyl)amino]-2-[4-(phenylmethyl)-1-piperazinyl]-3-pyridinecarboxylate;
1-methylethyl 4-{[4-(methyloxy)phenyl]amino}-2-[4-(phenylmethyl)-1-piperazinyl]-3-pyridinecarboxylate;
1-methylethyl 4-(phenylamino)-2-[4-(phenylmethyl)-1-piperazinyl]-3-pyridinecarboxylate; 1-methylethyl 2-[4-(phenylmethyl)-1-piperazinyl]-4-(phenylthio)-3-pyridinecarboxylate;
1-methylethyl 4-{[2-(methyloxy)phenyl]thio}-2-[4-(phenylmethyl)-1-piperazinyl]-3-pyridinecarboxylate; 1-methylethyl 2-[4-({2-[(2-chlorophenyl)amino]phenyl}methyl)-1-piperazinyl]-3-pyridinecarboxylate; 1-methylethyl 2-{4-[(3-{[2-(trifluoromethyl)phenyl]amino}phenyl)methyl]-1-piperazinyl}-3-pyridinecarboxylate;
1-methylethyl 2-{4-[(3-{[2-(methyloxy)phenyl]amino}phenyl)methyl]-1-piperazinyl}-3-pyridinecarboxylate;
1-methylethyl 2-[4-({3-[(2-methylphenyl)amino]phenyl}methyl)-1-piperazinyl]-3-pyridinecarboxylate;
1-methylethyl 2-[4-({3-[(2,6-difluorophenyl)amino]phenyl}methyl)-1-piperazinyl]-3-pyridinecarboxylate;
1-methylethyl 2-[4-({3-[(2-fluorophenyl)amino]phenyl}methyl)-1-piperazinyl]-3-pyridinecarboxylate;
1-methylethyl 2-[4-({3-[(2-chlorophenyl)amino]phenyl}methyl)-1-piperazinyl]-3-pyridinecarboxylate;
1-methylethyl 2-(4-{[4-({2-[(trifluoromethyl)oxy]phenyl}amino)phenyl)methyl}-1-piperazinyl)-3-pyridinecarboxylate;
1-methylethyl 2-(4-{[4-({3-(ethyloxy)carbonyl]phenyl}amino)phenyl]methyl}-1-piperazinyl)-3-pyridinecarboxylate;
1-methylethyl 2-{4-[(4-{[2-fluoro-6-(trifluoromethyl)phenyl]amino}phenyl)methyl]-1-piperazinyl}-3-pyridinecarboxylate;
1-methylethyl 2-[4-({4-[(2,6-difluorophenyl)amino]phenyl}methyl)-1-piperazinyl]-3-pyridinecarboxylate;
1-methylethyl 2-[4-({4-[(2-fluorophenyl)amino]phenyl}methyl)-1-piperazinyl]-3-pyridinecarboxylate;
1-methylethyl 2-[4-({4-[(2-chlorophenyl)amino]phenyl}methyl)-1-piperazinyl]-3-pyridinecarboxylate;
1-methylethyl 2-{4-[(4-{[4-(methyloxy)phenyl]amino}phenyl)methyl]-1-piperazinyl}-3-pyridinecarboxylate;
1-methylethyl 2-[4-(2-furanylmethyl)-1-piperazinyl]-4-phenyl-3-pyridinecarboxylate;
1-methylethyl 2-(4-{[2-(ethyloxy)phenyl]methyl}-1-piperazinyl)-4-phenyl-3-pyridinecarboxylate;
1-methylethyl 4-phenyl-2-[4-(2-thienylmethyl)-1-piperazinyl]-3-pyridinecarboxylate;
1-methylethyl 2-[4-(3-furanylmethyl)-1-piperazinyl]-4-phenyl-3-pyridinecarboxylate;
1-methylethyl 2-{4-[(5-methyl-2-thienyl)methyl]-1-piperazinyl}-4-phenyl-3-pyridinecarboxylate;
1-methylethyl 4-phenyl-2-(4-{[3-(phenyloxy)phenyl]methyl}-1-piperazinyl)-3-pyridinecarboxylate;
1-methylethyl 4-phenyl-2-(4-{[3-(phenyloxy)phenyl]methyl}-1-piperazinyl)-3-pyridinecarboxylate;
1-methylethyl 4-phenyl-2-[4-({3-[(phenylmethyl)oxy]phenyl}methyl)-1-piperazinyl]-3-pyridinecarboxylate;
1-methylethyl 4-phenyl-2-[4-({3-[(phenylmethyl)oxy]phenyl}methyl)-1-piperazinyl]-3-pyridinecarboxylate;
1-methylethyl 2-[4-({3-(methyloxy)-4-[(phenylmethyl)oxy]phenyl}methyl)-1-piperazinyl]-4-phenyl-3-pyridinecarboxylate;
1-methylethyl 2-[4-({3-(methyloxy)-4-[(phenylmethyl)oxy]phenyl}methyl)-1-piperazinyl]-4-phenyl-3-pyridinecarboxylate;
1-methylethyl 2-{4-[(2-cyanophenyl)methyl]-1-piperazinyl}-4-phenyl-3-pyridinecarboxylate;
1-methylethyl 4-phenyl-2-[4-({4-[(trifluoromethyl)oxy]phenyl}methyl)-1-piperazinyl]-3-pyridinecarboxylate;
1-methylethyl 4-phenyl-2-(4-{[4-(propyloxy)phenyl]methyl}-1-piperazinyl)-3-pyridinecarboxylate;
1-methylethyl 2-{4-[4-(2-methylphenyl)methyl]-1-piperazinyl}-4-phenyl-3-pyridinecarboxylate;
1-methylethyl 4-phenyl-2-[4-({2-[(phenylmethyl)oxy]phenyl}methyl)-1-piperazinyl]-3-pyridinecarboxylate;
1-methylethyl 2-[4-({4-(methyloxy)-3-[(phenylmethyl)oxy]phenyl}methyl)-1-piperazinyl]-4-phenyl-3-pyridinecarboxylate;
1-methylethyl 2-[4-(2-biphenylylmethyl)-1-piperazinyl]-4-phenyl-3-pyridinecarboxylate;
1-methylethyl 2-{4-[4-(3-fluoro-2-methylphenyl)methyl}-1-piperazinyl]-4-phenyl-3-pyridinecarboxylate;
1-methylethyl 2-[4-({2-[(1-methylethyl)oxy]phenyl}methyl)-1-piperazinyl]-4-phenyl-3-pyridinecarboxylate;
1-methylethyl 2-[4-({2-[(1-methylethyl)oxy]phenyl}methyl)-1-piperazinyl]-4-phenyl-3-pyridinecarboxylate;
1-methylethyl 2-{4-[(3-{[(2-chlorophenyl)methyl]oxy}phenyl)methyl]-1-piperazinyl}-4-phenyl-3-pyridinecarboxylate;
1-methylethyl 2-[4-({4-[(4-fluorophenyl)oxy]phenyl}methyl)-1-piperazinyl]-4-phenyl-3-pyridinecarboxylate;
1-methylethyl 2-(4-{[4-({[4-(ethyloxy)phenyl]oxy}methyl)phenyl]methyl}-1-piperazinyl)-3-pyridinecarboxylate;
1-methylethyl 2-(4-{[4-({[3-(methyloxy)phenyl]oxy}methyl)phenyl]methyl}-1-piperazinyl)-3-pyridinecarboxylate;
1-methylethyl 2-{4-[(4-{[(2,6-difluorophenyl)oxy]methyl}phenyl)methyl]-1-piperazinyl}-3-pyridinecarboxylate;
1-methylethyl 2-{4-[(4-{[(3,4-difluorophenyl)oxy]methyl}phenyl)methyl]-1-piperazinyl}-3-pyridinecarboxylate;
1-methylethyl 2-{4-[(4-{[(3-chloro-4-fluorophenyl)oxy]methyl}phenyl)methyl]-1-piperazinyl}-3-pyridinecarboxylate;
1-methylethyl 2-(4-{[4-({[4-(1,1-dimethylethyl)phenyl]oxy}methyl)phenyl]methyl}-1-piperazinyl)-3-pyridinecarboxylate;
1-methylethyl 2-(4-{[4-({[4-(methyloxy)phenyl]oxy}methyl)phenyl]methyl}-1-piperazinyl)-3-pyridinecarboxylate;
1-methylethyl 2-[4-({4-[({3-[(trifluoromethyl)oxy]phenyl}oxy)methyl]phenyl}methyl)-1-piperazinyl]-3-pyridinecarboxylate;
1-methylethyl 2-(4-{[4-({[2,3-bis(methyloxy)phenyl]oxy}methyl)phenyl]methyl}-1-piperazinyl)-3-pyridinecarboxylate;
1-methylethyl 2-{4-[(4-{[(2-chlorophenyl)oxy]methyl}phenyl)methyl]-1-piperazinyl}-3-pyridinecarboxylate;
1-methylethyl 2-(4-{[4-({[3,5-bis(methyloxy)phenyl]oxy}methyl)phenyl]methyl}-1-piperazinyl)-3-pyridinecarboxylate;
1-methylethyl 2-(4-{[4-({[2-(trifluoromethyl)phenyl]oxy}methyl)phenyl]methyl}-1-piperazinyl)-3-pyridinecarboxylate;
1-methylethyl 2-{4-[(4-{[(3-cyanophenyl)oxy]methyl}phenyl)methyl]-1-piperazinyl}-3-pyridinecarboxylate;
1-methylethyl 2-{4-[(4-{[(2,4-dichlorophenyl)oxy]methyl}phenyl)methyl]-1-piperazinyl}-3-pyridinecarboxylate;
1-methylethyl 2-{4-[(4-{[(2-methylphenyl)oxy]methyl}phenyl)methyl]-1-piperazinyl}-3-pyridinecarboxylate;
1-methylethyl 2-{4-[(4-{[(4-methylphenyl)oxy]methyl}phenyl)methyl]-1-piperazinyl}-3-pyridinecarboxylate;
1-methylethyl 2-{4-[(4-{[(4-fluorophenyl)oxy]methyl}phenyl)methyl]-1-piperazinyl}-3-pyridinecarboxylate;
1-methylethyl 2-{4-[(4-{[(4-cyanophenyl)oxy]methyl}phenyl)methyl]-1-piperazinyl}-3-pyridinecarboxylate;
1-methylethyl 2-(4-{[3-({[4-(ethyloxy)phenyl]oxy}methyl)phenyl]methyl}-1-piperazinyl)-3-pyridinecarboxylate;
1-methylethyl 2-(4-{[3-({[3-(methyloxy)phenyl]oxy}methyl)phenyl]methyl}-1-piperazinyl)-3-pyridinecarboxylate;
1-methylethyl 2-{4-[(3-{[(2,6-difluorophenyl)oxy]methyl}phenyl)methyl]-1-piperazinyl}-3-pyridinecarboxylate;
1-methylethyl 2-{4-[(3-{[(3,4-difluorophenyl)oxy]methyl}phenyl)methyl]-1-piperazinyl}-3-pyridinecarboxylate;
1-methylethyl 2-{4-[(3-{[(3-chloro-4-fluorophenyl)oxy]methyl}phenyl)methyl]-1-piperazinyl}-3-pyridinecarboxylate;
1-methylethyl 2-(4-{[3-({[4-(1,1-dimethylethyl)phenyl]oxy}methyl)phenyl]methyl}-1-piperazinyl)-3-pyridinecarboxylate;
1-methylethyl 2-(4-{[3-({[4-(methyloxy)phenyl]oxy}methyl)phenyl]methyl}-1-piperazinyl)-3-pyridinecarboxylate;
1-methylethyl 2-(4-{[3-({[2,3-bis(methyloxy)phenyl]oxy}methyl)phenyl]methyl}-1-piperazinyl)-3-pyridinecarboxylate;
1-methylethyl 2-{4-[(3-{[(2-chlorophenyl)oxy]methyl}phenyl)methyl]-1-piperazinyl}-3-pyridinecarboxylate;
1-methylethyl 2-(4-{[3-({[3,5-bis(methyloxy)phenyl]oxy}methyl)phenyl]methyl}-1-piperazinyl)-3-pyridinecarboxylate;
1-methylethyl 2-(4-{[3-({[2-(trifluoromethyl)phenyl]oxy}methyl)phenyl]methyl}-1-piperazinyl)-3-pyridinecarboxylate;
1-methylethyl 2-{4-[(3-{[(3-cyanophenyl)oxy]methyl}phenyl)methyl]-1-piperazinyl}-3-pyridinecarboxylate;
1-methylethyl 2-{4-[(3-{[(2,4-dichlorophenyl)oxy]methyl}phenyl)methyl]-1-piperazinyl}-3-pyridinecarboxylate;
1-methylethyl 2-{4-[(3-{[(4-methylphenyl)oxy]methyl}phenyl)methyl]-1-piperazinyl}-3-pyridinecarboxylate;
1-methylethyl 2-{4-[(3-{[(4-fluorophenyl)oxy]methyl}phenyl)methyl]-1-piperazinyl}-3-pyridinecarboxylate;
1-methylethyl 2-(4-{[3-({[2-(ethyloxy)phenyl]oxy}methyl)phenyl]methyl}-1-piperazinyl)-3-pyridinecarboxylate;
1-methylethyl 2-{4-[(3-{[(4-cyanophenyl)oxy]methyl}phenyl)methyl]-1-piperazinyl}-3-pyridinecarboxylate;
1-methylethyl 2-(4-[(4-{[ethyl(3-furanylmethyl)amino]methyl}phenyl)methyl]-1-piperazinyl)-3-pyridinecarboxylate;
1-methylethyl 2-[4-({4-[(ethyl{[3-(ethyloxy)phenyl]methyl}amino)methyl]phenyl}methyl)-1-piperazinyl]-3-pyridinecarboxylate;
1-methylethyl 2-(4-{[4-({ethyl[(5-methyl-2-thienyl)methyl]amino}methyl)phenyl]methyl}-1-piperazinyl)-3-pyridinecarboxylate;
1-methylethyl 2-{4-[(4-{[[(2-chloro-6-fluorophenyl)methyl](ethyl)amino]methyl}phenyl)methyl]-1-piperazinyl}-3-pyridinecarboxylate;
1-methylethyl 2-[4-({4-[(ethyl{[2-(ethyloxy)phenyl]methyl)amino}methyl]phenyl]methyl)-1-piperazinyl}-3-pyridinecarboxylate;
1-methylethyl 2-[4-({4-[(ethyl{[3-(methyloxy)phenyl]methyl}amino) methyl]phenyl}methyl)-1-piperazinyl]-3-pyridinecarboxylate;
1-methylethyl 2-{4-[(4-{[ethyl(2-furanylmethyl)amino]methyl}phenyl)methyl]-1-piperazinyl}-3-pyridinecarboxylate;
1-methylethyl 2-{4-[(4-{[ethyl(2-thienylmethyl)amino]methyl}phenyl)methyl]-1-piperazinyl}-3-pyridinecarboxylate;
1-methylethyl 4-methyl-2-[4-({4-[(methyloxy)carbonyl]phenyl}methyl)-1-piperazinyl]-3-pyridinecarboxylate;
1-methylethyl 4-methyl-2-[4-(phenylmethyl)-1-piperazinyl]-3-pyridinecarboxylate;
1-methylethyl 4-methyl-2-(4-{[4-(methyloxy)phenyl]methyl}-1-piperazinyl)-3-pyridinecarboxylate;
1-methylethyl 2-{4-[(2-cyanophenyl)methyl]-1-piperazinyl}-4-methyl-3-pyridinecarboxylate;
1-methylethyl 2-[4-(2-furanylmethyl)-1-piperazinyl]-4-methyl-3-pyridinecarboxylate;
1-methylethyl 2-{4-[4-(3-fluorophenyl)methyl]-1-piperazinyl}-4-methyl-3-pyridinecarboxylate;
1-methylethyl 4-methyl-2-(4-{[3-(methyloxy)phenyl]methyl}-1-piperazinyl)-3-pyridinecarboxylate;
1-methylethyl 2-[4-(3-furanylmethyl)-1-piperazinyl]-4-methyl-3-pyridinecarboxylate;
1-methylethyl 4-methyl-2-{4-[4-(5-methyl-2-thienyl)methyl}-1-piperazinyl]-3-pyridinecarboxylate;
1-methylethyl 2-{4-[(4-cyanophenyl)methyl]-1-piperazinyl}-4-methyl-3-pyridinecarboxylate;
1-methylethyl 2-{4-[(3-cyanophenyl)methyl]-1-piperazinyl}-4-methyl-3-pyridinecarboxylate;
1-methylethyl 2-{4-[(3-cyano-4-fluorophenyl)methyl]-1-piperazinyl}-4-methyl-3-pyridinecarboxylate;
1-methylethyl 2-{4-[(1,3-dimethyl-1H-pyrazol-4-yl)methyl]-1-piperazinyl}-4-methyl-3-pyridinecarboxylate;
1-methylethyl 2-{4-[(3,5-dimethyl-4-isoxazolypmethyl]-1-piperazinyl}-4-methyl-3-pyridinecarboxylate;
1-methylethyl 2-(4-{[4-(acetylamino)phenyl]methyl}-1-piperazinyl)-4-methyl-3-pyridinecarboxylate;
1-methylethyl 2-(4-{[4-(acetyloxy)phenyl]methyl}-1-piperazinyl)-4-methyl-3-pyridinecarboxylate;
1-methylethyl 4-methyl-2-(4-{[1-(3-pyridinyl)-1H-pyrrol-2-yl]methyl}-1-piperazinyl)-3-pyridinecarboxylate;
1-methylethyl 4-methyl-2-(4-{[4-(1H-tetrazol-5-yl)phenyl]methyl}-1-piperazinyl)-3-pyridinecarboxylate;
1-methylethyl 4-methyl-2-(4-{[4-(methylsulfonyl)phenyl]methyl}-1-piperazinyl)-3-pyridinecarboxylate;
1-methylethyl 2-(4-{[2-[(cyanomethyl)oxy]-3-(methyloxy)phenyl]methyl}-1-piperazinyl)-4-methyl-3-pyridinecarboxylate;
1-methylethyl 4-methyl-2-[4-({1,2,5-trimethyl-4-[(methyloxy)carbonyl]-1H-pyrrol-3-yl]methyl)-1-piperazinyl}-3-pyridinecarboxylate;
1-methylethyl 4-methyl-2-(4-{[2-(1-piperidinyl)-1,3-thiazol-5-yl]methyl}-1-piperazinyl)-3-pyridinecarboxylate;
1-methylethyl 4-methyl-2-(4-{[2-(4-morpholinyl)-1,3-thiazol-5-yl]methyl}-1-piperazinyl)-3-pyridinecarboxylate;
1-methylethyl 4-methyl-2-(4-{[2-(4-methyl-1-piperazinyl)-1,3-thiazol-5-yl]methyl}-1-piperazinyl)-3-pyridinecarboxylate;
1-methylethyl 2-[4-({1-[3-cyano-4-(methyloxy)-2-pyridinyl]-1H-pyrrol-2-yl]methyl)-1-piperazinyl}-4-methyl-3-pyridinecarboxylate;
1-methylethyl 2-(4-{[4-({[3-(trifluoromethyl)phenyl]methyl}oxy)phenyl]methyl}-1-piperazinyl)-3-pyridinecarboxylate;
1-methylethyl 2-{4-[(4-{[(3-bromophenyl)methyl]oxy}phenyl)methyl]-1-piperazinyl}-3-pyridinecarboxylate;
1-methylethyl 2-(4-{[4-{[(2,4-dichlorophenyl)methyl]oxy}-3-(methyloxy)phenyl]methyl}-1-piperazinyl)-3-pyridinecarboxylate;
1-methylethyl 2-[4-({3,5-bis(methyloxy)-4-[(phenylmethyl)oxy]phenyl}methyl)-1-piperazinyl]-3-pyridinecarboxylate;
1-methylethyl 2-[4-({4-(methyloxy)-3-[(phenylmethyl)oxy]phenyl}methyl)-1-piperazinyl]-3-pyridinecarboxylate;
1-methylethyl 2-(4-{[4-{[(4-chlorophenyl)methyl]oxy}-3-(ethyloxy)phenyl]methyl}-1-piperazinyl)-3-pyridinecarboxylate;
1-methylethyl 2-(4-{[4-{[(2-chlorophenyl)methyl]oxy}-3-(methyloxy)phenyl]methyl}-1-piperazinyl)-3-pyridinecarboxylate;
1-methylethyl 2-(4-{[4-{[(2-chlorophenyl)methyl]oxy}-3-(ethyloxy)phenyl]methyl}-1-piperazinyl)-3-pyridinecarboxylate;
1-methylethyl 2-{4-[(4-{[(3-fluorophenyl)methyl]oxy}phenyl)methyl]-1-piperazinyl}-3-pyridinecarboxylate;
1-methylethyl 2-[4-({3-chloro-4-[(phenylmethyl)oxy]phenyl}methyl)-1-piperazinyl]-3-pyridinecarboxylate;
1-methylethyl 2-[4-({2-methyl-4-[(phenylmethyl)oxy]phenyl}methyl)-1-piperazinyl]-3-pyridinecarboxylate;
1-methylethyl 2-{4-[(4-{[(2-chlorophenyl)methyl]oxy}phenyl)methyl]-1-piperazinyl}-3-pyridinecarboxylate;
1-methylethyl 2-[4-({3,5-bis[(phenylmethyl)oxy]phenyl}methyl)-1-piperazinyl]-3-pyridinecarboxylate;
1-methylethyl 2-{4-[(4-{[(4-fluorophenyl)methyl]oxy}phenyl)methyl]-1-piperazinyl}-3-pyridinecarboxylate;
1-methylethyl 2-{4-[(4-{[(2,4-dichlorophenyl)methyl]oxy}phenyl)methyl]-1-piperazinyl}-3-pyridinecarboxylate;
1-methylethyl 2-(4-{[4-{[(4-fluorophenyl)methyl]oxy}-3-(methyloxy)phenyl]methyl}-1-piperazinyl)-3-pyridinecarboxylate;
1-methylethyl 2-[4-({3-(ethyloxy)-4-[(phenylmethyl)oxy]phenyl}methyl)-1-piperazinyl]-3-pyridinecarboxylate;
1-methylethyl 2-[4-({3-(methyloxy)-2-[(phenylmethyl)oxy]phenyl}methyl)-1-piperazinyl]-3-pyridinecarboxylate;
1-methylethyl 2-[4-({4,5-bis(methyloxy)-2-[(phenylmethyl)oxy]phenyl}methyl)-1-piperazinyl]-3-pyridinecarboxylate;
1-methylethyl 2-[4-({4-[(phenylmethyl)oxy]phenyl}methyl)-1-piperazinyl]-3-pyridinecarboxylate;
1-methylethyl 2-[4-({3,5-dimethyl-4-[(phenylmethyl)oxy]phenyl}methyl)-1-piperazinyl]-3-pyridinecarboxylate;
1-methylethyl 2-[4-({2-hydroxy-4-[(phenylmethyl)oxy]phenyl}methyl)-1-piperazinyl]-3-pyridinecarboxylate;
1-methylethyl 2-{4-[(4-{[(3,4-dichlorophenyl)methyl]oxy}phenyl)methyl]-1-piperazinyl}-3-pyridinecarboxylate;
1-methylethyl 2-(4-{[4-{[(2-chloro-6-fluorophenyl)methyl]oxy}-3-(methyloxy)phenyl]methyl}-1-piperazinyl)-3-pyridinecarboxylate;
1-methylethyl 2-(4-{[4-{[(4-chlorophenyl)methyl]oxy}-3-(methyloxy)phenyl]methyl}-1-piperazinyl)-3-pyridinecarboxylate;
1-methylethyl 2-(4-{[3-(methyloxy)-4-({[4-(methyloxy)phenyl]methyl}oxy)phenyl]methyl}-1-piperazinyl)-3-pyridinecarboxylate;
1-methylethyl 2-[4-({2-(methyloxy)-4-[(phenylmethyl)oxy]phenyl}methyl)-1-piperazinyl]-3-pyridinecarboxylate;
1-methylethyl 2-{4-[(4-{[(4-bromophenyl)methyl]oxy}phenyl)methyl]-1-piperazinyl}-3-pyridinecarboxylate;
1-methylethyl 2-[4-({2-[(phenylmethyl)oxy]phenyl}methyl)-1-piperazinyl]-3-pyridinecarboxylate;
1-methylethyl 2-[4-({3,4-bis[(phenylmethyl)oxy]phenyl}methyl)-1-piperazinyl]-3-pyridinecarboxylate;
1-methylethyl 2-[4-({3-(methyloxy)-4-[(phenylmethyl)oxy]phenyl}methyl)-1-piperazinyl]-3-pyridinecarboxylate;
1-methylethyl 2-{4-[(4-{[(2-chloro-6-fluorophenyl)methyl]oxy}phenyl)methyl]-1-piperazinyl}-3-pyridinecarboxylate;
1-methylethyl 2-[4-({4-[(4-bromophenyl)oxy]phenyl}methyl)-1-piperazinyl]-3-pyridinecarboxylate;
1-methylethyl 2-[4-({3-[(3,5-dichlorophenyl)oxy]phenyl}methyl)-1-piperazinyl]-3-pyridinecarboxylate;
1-methylethyl 2-[4-({3-[(4-methylphenyl)oxy]phenyl}methyl)-1-piperazinyl]-3-pyridinecarboxylate;
1-methylethyl 2-[4-(2-biphenylylmethyl)-1-piperazinyl]-3-pyridinecarboxylate;
1-methylethyl 2-[4-({4-[(3-chlorophenyl)oxy]phenyl}methyl)-1-piperazinyl]-3-pyridinecarboxylate;
1-methylethyl 2-(4-{[4-fluoro-3-(phenyloxy)phenyl]methyl}-1-piperazinyl)-3-pyridinecarboxylate;
1-methylethyl 2-[4-({3-[(4-chlorophenyl)oxy]phenyl}methyl)-1-piperazinyl]-3-pyridinecarboxylate;
1-methylethyl 2-[4-(9H-fluoren-2-ylmethyl)-1-piperazinyl]-3-pyridinecarboxylate;
1-methylethyl 2-[4-(4-biphenylylmethyl)-1-piperazinyl]-3-pyridinecarboxylate;
1-methylethyl 2-[4-({4-[(4-methylphenyl)oxy]phenyl}methyl)-1-piperazinyl]-3-pyridinecarboxylate;
1-methylethyl 2-(4-{[3-(phenyloxy)phenyl]methyl}-1-piperazinyl)-3-pyridinecarboxylate;
1-methylethyl 2-[4-({3-[(3,4-dichlorophenyl)oxy]phenyl}methyl)-1-piperazinyl]-3-pyridinecarboxylate;
1-methylethyl 2-{4-[(4′-methyl-3-biphenylyl)methyl]-1-piperazinyl}-3-pyridinecarboxylate;
1-methylethyl 2-[4-({2-[(4-cyanophenyl)oxy]phenyl}methyl)-1-piperazinyl]-3-pyridinecarboxylate;
1-methylethyl 2-{4-[(4′-methyl-4-biphenylyl)methyl]-1-piperazinyl}-3-pyridinecarboxylate;
1-methylethyl 2-[4-({4-[(4-fluorophenyl)oxy]phenyl}methyl)-1-piperazinyl]-3-pyridinecarboxylate;
1-methylethyl 2-{4-[(9-ethyl-9H-carbazol-3-yl)methyl]-1-piperazinyl}-3-pyridinecarboxylate;
1-methylethyl 2-[4-(dibenzo[b,d]furan-4-ylmethyl)-1-piperazinyl]-3-pyridinecarboxylate;
1-methylethyl 2-[4-({4-[(4-chlorophenyl)oxy]phenyl}methyl)-1-piperazinyl]-3-pyridinecarboxylate;
1-methylethyl 2-{4-[(4′-chloro-3-biphenylyl)methyl]-1-piperazinyl}-3-pyridinecarboxylate;
1-methylethyl 2-{4-[(2-{[4-(methyloxy)phenyl]oxy}phenyl)methyl]-1-piperazinyl}-3-pyridinecarboxylate;
1-methylethyl 2-[4-({4-[(2,4-dichlorophenyl)oxy]phenyl}methyl)-1-piperazinyl]-3-pyridinecarboxylate;
1-methylethyl 2-{4-[(4-{[4-(methyloxy)phenyl]oxy}phenyl)methyl]-1-piperazinyl}-3-pyridinecarboxylate;
1-methylethyl 2-[4-({2-[(4-fluorophenyl)oxy]phenyl}methyl)-1-piperazinyl]-3-pyridinecarboxylate;
1-methylethyl 2-[4-({2-[(4-chlorophenyl)oxy]phenyl}methyl)-1-piperazinyl]-3-pyridinecarboxylate;
1-methylethyl 2-[4-({4′-[(methyloxy)carbonyl]-3-biphenylyl]methyl)-1-piperazinyl}-3-pyridinecarboxylate;
1-methylethyl 2-[4-({4′-[(methyloxy)carbonyl]-4-biphenylyl]methyl)-1-piperazinyl}-3-pyridinecarboxylate;
1-methylethyl 2-[4-({4-[(4-cyanophenyl)oxy]phenyl}methyl)-1-piperazinyl]-3-pyridinecarboxylate;
1-methylethyl 2-{4-[(3-{[4-(methyloxy)phenyl]oxy}phenyl)methyl]-1-piperazinyl}-3-pyridinecarboxylate;
1-methylethyl 2-(4-{[4-(phenyloxy)phenyl]methyl}-1-piperazinyl)-3-pyridinecarboxylate;
1-methylethyl 2-{4-[(3-{[4-(1,1-dimethylethyl)phenyl]oxy}phenyl)methyl]-1-piperazinyl}-3-pyridinecarboxylate;
1-methylethyl 2-(4-{[2′-(trifluoromethyl)-3-biphenylyl]methyl}-1-piperazinyl)-3-pyridinecarboxylate;
1-methylethyl 2-[4-({2-[(4-chlorophenyl)thio]phenyl}methyl)-1-piperazinyl]-3-pyridinecarboxylate;
1-methylethyl 2-(4-{[2′-(trifluoromethyl)-4-biphenylyl]methyl}-1-piperazinyl)-3-pyridinecarboxylate;
1-methylethyl 2-(4-{[3′-(methyloxy)-2-biphenylyl]methyl}-1-piperazinyl)-3-pyridinecarboxylate;
1-methylethyl 2-{4-[(3-{[3-(trifluoromethyl)phenyl]oxy}phenyl)methyl]-1-piperazinyl}-3-pyridinecarboxylate;
1-methylethyl 2-(4-{[2-(phenyloxy)phenyl]methyl}-1-piperazinyl)-3-pyridinecarboxylate;
1-methylethyl 2-(4-{[3-({[3,4-bis(methyloxy)phenyl]methyl}oxy)phenyl]methyl}-1-piperazinyl)-3-pyridinecarboxylate;
1-methylethyl 2-(4-{[3-({[4-(1,1-dimethylethyl)phenyl]methyl}oxy)phenyl]methyl}-1-piperazinyl)-3-pyridinecarboxylate;
1-methylethyl 2-(4-{[3-({[3,5-bis(methyloxy)phenyl]methyl}oxy)phenyl]methyl}-1-piperazinyl)-3-pyridinecarboxylate;
1-methylethyl 2-{4-[(3-{[(2,4,5-trifluorophenyl)methyl]oxy}phenyl)methyl]-1-piperazinyl}-3-pyridinecarboxylate;
1-methylethyl 2-[4-({3-[(2,3-dihydro-1,4-benzodioxin-5-ylmethyl)oxy]phenyl}methyl)-1-piperazinyl]-3-pyridinecarboxylate;
1-methylethyl 2-(4-{[3-({[2-(methyloxy)phenyl]methyl}oxy)phenyl]methyl}-1-piperazinyl)-3-pyridinecarboxylate;
1-methylethyl 2-{4-[(3-{[(2,6-difluorophenyl)methyl]oxy}phenyl)methyl]-1-piperazinyl}-3-pyridinecarboxylate;
1-methylethyl 2-{4-[(3-{[(3,5-dimethylphenyl)methyl]oxy}phenyl)methyl]-1-piperazinyl}-3-pyridinecarboxylate;
1-methylethyl 2-(4-{[3-({[3-(dimethylamino)phenyl]methyl}oxy)phenyl]methyl}-1-piperazinyl)-3-pyridinecarboxylate;
1-methylethyl 2-{4-[(3-{[(2,4-difluorophenyl)methyl]oxy}phenyl)methyl]-1-piperazinyl}-3-pyridinecarboxylate;
1-methylethyl 2-{4-[(3-{[(2,3-difluorophenyl)methyl]oxy}phenyl)methyl]-1-piperazinyl}-3-pyridinecarboxylate;
1-methylethyl 2-(4-{[3-({[4-(butyloxy)phenyl]methyl}oxy)phenyl]methyl}-1-piperazinyl)-3-pyridinecarboxylate;
1-methylethyl 2-(4-{[3-({[4-(ethyloxy)phenyl]methyl}oxy)phenyl]methyl}-1-piperazinyl)-3-pyridinecarboxylate;
1-methylethyl 2-{4-[(3-{[(4-ethylphenyl)methyl]oxy}phenyl)methyl]-1-piperazinyl}-3-pyridinecarboxylate;
1-methylethyl 2-(4-{[3-({[2-fluoro-6-(methyloxy)phenyl]methyl}oxy)phenyl]methyl}-1-piperazinyl)-3-pyridinecarboxylate;
1-methylethyl 2-{4-[(3-{[(4-cyanophenyl)methyl]oxy}phenyl)methyl]-1-piperazinyl}-3-pyridinecarboxylate;
1-methylethyl 2-{4-[(3-{[(2,4-dimethylphenyl)methyl]oxy}phenyl)methyl]-1-piperazinyl}-3-pyridinecarboxylate;
1-methylethyl 2-(4-{[3-({[4-fluoro-3-(methyloxy)phenyl]methyl}oxy)phenyl]methyl}-1-piperazinyl)-3-pyridinecarboxylate;
1-methylethyl 2-[4-({3-[(1-naphthalenylmethyl)oxy]phenyl}methyl)-1-piperazinyl]-3-pyridinecarboxylate;
1-methylethyl 2-(4-{[3-({[4-(methylsulfonyl)phenyl]methyl}oxy)phenyl]methyl}-1-piperazinyl)-3-pyridinecarboxylate;
1-methylethyl 2-{4-[(3-{[(3,5-dichlorophenyl)methyl]oxy}phenyl)methyl]-1-piperazinyl}-3-pyridinecarboxylate;
1-methylethyl 2-{4-[(3-{[(2,3-dichlorophenyl)methyl]oxy}phenyl)methyl]-1-piperazinyl}-3-pyridinecarboxylate;
1-methylethyl 2-[4-({3-[({4-[(methyloxy)carbonyl]phenyl}methyl)oxy]phenyl}methyl)-1-piperazinyl]-3-pyridinecarboxylate;
1-methylethyl 2-(4-{[3-({[4-chloro-2-(methyloxy)phenyl]methyl}oxy)phenyl]methyl}-1-piperazinyl)-3-pyridinecarboxylate;
1-methylethyl 2-(4-{[3-({[4-(methyloxy)phenyl]methyl}oxy)phenyl]methyl}-1-piperazinyl)-3-pyridinecarboxylate;
1-methylethyl 2-(4-{[3-({[4-(1-methylethyl)phenyl]methyl}oxy)phenyl]methyl}-1-piperazinyl)-3-pyridinecarboxylate;
1-methylethyl 2-(4-{[3-({[2,5-bis(methyloxy)phenyl]methyl}oxy)phenyl]methyl}-1-piperazinyl)-3-pyridinecarboxylate;
1-methylethyl 2-(4-{[3-({[2,4-bis(methyloxy)phenyl]methyl}oxy)phenyl]methyl}-1-piperazinyl)-3-pyridinecarboxylate;
1-methylethyl 2-(4-{[4-({[3,4-bis(methyloxy)phenyl]methyl}oxy)phenyl]methyl}-1-piperazinyl)-3-pyridinecarboxylate;
1-methylethyl 2-(4-{[4-({[4-(1,1-dimethylethyl)phenyl]methyl}oxy)phenyl]methyl}-1-piperazinyl)-3-pyridinecarboxylate;
1-methylethyl 2-{4-[(4-{[(3-chlorophenyl)methyl]oxy}phenyl)methyl]-1-piperazinyl}-3-pyridinecarboxylate;
1-methylethyl 2-(4-{[4-({[3,5-bis(methyloxy)phenyl]methyl}oxy)phenyl]methyl}-1-piperazinyl)-3-pyridinecarboxylate;
1-methylethyl 2-{4-[(4-{[(2,4,5-trifluorophenyl)methyl]oxy}phenyl)methyl]-1-piperazinyl}-3-pyridinecarboxylate;
1-methylethyl 2-[4-({4-[(2,3-dihydro-1,4-benzodioxin-5-ylmethyl)oxy]phenyl}methyl)-1-piperazinyl]-3-pyridinecarboxylate;
1-methylethyl 2-(4-{[4-({[2-(methyloxy)phenyl]methyl}oxy)phenyl]methyl}-1-piperazinyl)-3-pyridinecarboxylate;
1-methylethyl 2-{4-[(4-{[(2,6-difluorophenyl)methyl]oxy}phenyl)methyl]-1-piperazinyl}-3-pyridinecarboxylate;
1-methylethyl 2-{4-[(4-{[(3,5-dimethyl phenyl)methyl]oxy}phenyl)methyl]-1-piperazinyl}-3-pyridinecarboxylate;
1-methylethyl 2-{4-[(4-{[(2-ethylphenyl)methyl]oxy}phenyl)methyl]-1-piperazinyl}-3-pyridinecarboxylate;
1-methylethyl 2-(4-{[4-({[3-(dimethylamino)phenyl]methyl}oxy)phenyl]methyl}-1-piperazinyl)-3-pyridinecarboxylate;
1-methylethyl 2-{4-[(4-{[(2,4-difluorophenyl)methyl]oxy}phenyl)methyl]-1-piperazinyl}-3-pyridinecarboxylate;
1-methylethyl 2-{4-[(4-{[(3-methylphenyl)methyl]oxy}phenyl)methyl]-1-piperazinyl}-3-pyridinecarboxylate;
1-methylethyl 2-{4-[(4-{[(3,4-difluorophenyl)methyl]oxy}phenyl)methyl]-1-piperazinyl}-3-pyridinecarboxylate;
1-methylethyl 2-(4-{[4-({[4-(butyloxy)phenyl]methyl}oxy)phenyl]methyl}-1-piperazinyl)-3-pyridinecarboxylate;
1-methylethyl 2-(4-{[4-({[4-(ethyloxy)phenyl]methyl}oxy)phenyl]methyl}-1-piperazinyl)-3-pyridinecarboxylate;
1-methylethyl 2-{4-[(4-{[(4-ethylphenyl)methyl]oxy}phenyl)methyl]-1-piperazinyl}-3-pyridinecarboxylate;
1-methylethyl 2-(4-{[4-({[2-fluoro-6-(methyloxy)phenyl]methyl}oxy)phenyl]methyl}-1-piperazinyl)-3-pyridinecarboxylate;
1-methylethyl 2-{4-[(4-{[(5-chloro-2-fluorophenyl)methyl]oxy}phenyl)methyl]-1-piperazinyl}-3-pyridinecarboxylate;
1-methylethyl 2-{4-[(4-{[(4-cyanophenyl)methyl]oxy}phenyl)methyl]-1-piperazinyl}-3-pyridinecarboxylate;
1-methylethyl 2-{4-[(4-{[(4-methylphenyl)methyl]oxy}phenyl)methyl]-1-piperazinyl}-3-pyridinecarboxylate;
1-methylethyl 2-{4-[(4-{[(2,6-dichlorophenyl)methyl]oxy}phenyl)methyl]-1-piperazinyl}-3-pyridinecarboxylate;
1-methylethyl 2-(4-{[4-({[2-(ethyloxy)phenyl]methyl}oxy)phenyl]methyl}-1-piperazinyl)-3-pyridinecarboxylate;
1-methylethyl 2-{4-[(4-{[(2,4-dimethylphenyl)methyl]oxy}phenyl)methyl]-1-piperazinyl}-3-pyridinecarboxylate;
1-methylethyl 2-(4-{[4-({[4-fluoro-3-(methyloxy)phenyl]methyl}oxy)phenyl]methyl}-1-piperazinyl)-3-pyridinecarboxylate;
1-methylethyl 2-[4-({4-[(1-naphthalenylmethyl)oxy]phenyl}methyl)-1-piperazinyl]-3-pyridinecarboxylate;
1-methylethyl 2-(4-{[4-({[4-(methylsulfonyl)phenyl]methyl}oxy)phenyl]methyl}-1-piperazinyl)-3-pyridinecarboxylate;
1-methylethyl 2-[4-({4-[(2-biphenylylmethyl)oxy]phenyl}methyl)-1-piperazinyl]-3-pyridinecarboxylate;
1-methylethyl 2-{4-[(4-{[(3,5-dichlorophenyl)methyl]oxy}phenyl)methyl]-1-piperazinyl}-3-pyridinecarboxylate;
1-methylethyl 2-{4-[(4-{[(2,3-dichlorophenyl)methyl]oxy}phenyl)methyl]-1-piperazinyl}-3-pyridinecarboxylate;
1-methylethyl 2-[4-({4-[({4-[(methyloxy)carbonyl]phenyl}methyl)oxy]phenyl}methyl)-1-piperazinyl]-3-pyridinecarboxylate;
1-methylethyl 2-(4-{[4-({[4-chloro-2-(methyloxy)phenyl]methyl}oxy)phenyl]methyl}-1-piperazinyl)-3-pyridinecarboxylate;
1-methylethyl 2-(4-{[4-({[4-(methyloxy)phenyl]methyl}oxy)phenyl]methyl}-1-piperazinyl)-3-pyridinecarboxylate;
1-methylethyl 2-{4-[(4-{[(2-methylphenyl)methyl]oxy}phenyl)methyl]-1-piperazinyl}-3-pyridinecarboxylate;
1-methylethyl 2-(4-{[4-({[4-(1-methylethyl)phenyl]methyl}oxy)phenyl]methyl}-1-piperazinyl)-3-pyridinecarboxylate;
1-methylethyl 2-[4-({4-[(4-biphenylylmethyl)oxy]phenyl}methyl)-1-piperazinyl]-3-pyridinecarboxylate;
1-methylethyl 2-(4-{[4-({[2,5-bis(methyloxy)phenyl]methyl}oxy)phenyl]methyl}-1-piperazinyl)-3-pyridinecarboxylate;
1-methylethyl 2-(4-{[4-({[3-(methyloxy)phenyl]methyl}oxy)phenyl]methyl}-1-piperazinyl)-3-pyridinecarboxylate;
1-methylethyl 2-(4-{[4-({[2,4-bis(methyloxy)phenyl]methyl}oxy)phenyl]methyl}-1-piperazinyl)-3-pyridinecarboxylate;
1-methylethyl 2-(4-{[4-({[2-(trifluoromethyl)phenyl]methyl}oxy)phenyl]methyl}-1-piperazinyl)-3-pyridinecarboxylate;
1-methylethyl 2-(4-{[4-({4-[(2-chloro-6-fluorophenyl)methyl]-1-piperazinyl]methyl)phenyl}methyl}-1-piperazinyl)-3-pyridinecarboxylate;
1-methylethyl 2-{4-[(4-{[4-(phenylmethyl)-1-piperazinyl]methyl}phenyl)methyl]-1-piperazinyl}-3-pyridinecarboxylate; 1-methylethyl 2-{4-[4-(4-{[4-(2-pyridinylmethyl)-1-piperazinyl]methyl}phenyl)methyl}-1-piperazinyl]-3-pyridinecarboxylate; 1-methylethyl 2-[4-({4-[(4-{[3-(methyloxy)phenyl]methyl}-1-piperazinyl)methyl]phenyl}methyl)-1-piperazinyl]-3-pyridinecarboxylate; 1-methylethyl 2-[4-({4-[(4-{[4-(methyloxy)phenyl]methyl}-1-piperazinyl)methyl]phenyl}methyl)-1-piperazinyl]-3-pyridinecarboxylate;
1-Methylethyl-2-{4-[(3-{[4-(methyloxy)phenyl]oxy}phenyl)methyl]-1-piperazinyl}-3-pyridine carboxylate dihydrochloride;
1-Methylethyl-2-(4-{[Z-(trifluoromethyl)-3-biphenylyl]methyl}-1-piperazinyl)-3-pyridine carboxylate; 1-Methylethyl-2-(4-{[3-({[2-(methyloxy)phenyl]methyl}oxy)phenyl]methyl}-1-piperazinyl)-3-pyridinecarboxylate hydrochloride;
1-Methylethyl-2-(4-{[3-({[4-(ethyloxy)phenyl]methyl}oxy)phenyl]methyl}-1-piperazinyl)-3-pyridinecarboxylate;
1-Methylethyl 2-[4-({4-[(ethylamino)methyl]phenyl}methyl)-1-piperazinyl]-3-pyridine carboxylate dihydrochloride;
1-Methylethyl 2-{4-[(4-{[[(2-chloro-6-fluorophenyl)methyl](ethyl)amino]methyl}phenyl)methyl]-1-piperazinyl}-3-pyridinecarboxylate;
1-[(4-{[[(2-Chloro-6-fluorophenyl)methyl](ethyl)ammonio]methyl}phenyl)methyl]-4-(3-{[(1-methylethyl)oxy]carbonyl}-2-pyridinyl)piperazin-1-ium di-maleate; 1-methylethyl 2-(4-{[4-({ethyl[(2-{[(1-methylethyl)oxy]carbonyl]phenyl)methyl]amino}methyl)phenyl}methyl}-1-piperazinyl)-3-pyridinecarboxylate dihydrochloride;
1-methylethyl 2-(4-{[4-({ethyl[(3-{[(1-methylethyl)oxy]carbonyl]phenyl)methyl]amino}methyl)phenyl}methyl}-1-piperazinyl)-3-pyridinecarboxylate dihydrochloride;
1-methylethyl 2-(4-{[4-({ethyl[(4-{[(1-methylethyl)oxy]carbonyl]phenyl)methyl]amino}methyl)phenyl}methyl}-1-piperazinyl)-3-pyridinecarboxylate;
1-methylethyl2-[4-({2-[(dimethylamino)sulfonyl]phenyl}methyl)-1-piperazinyl]-3-pyridine carboxylate hydrochloride;
1-Methylethyl2-[4-({3-[(dimethylamino)sulfonyl]phenyl}methyl)-1-piperazinyl]-3-pyridine carboxylate;
1-methylethyl2-[4-({4-[(dimethylamino)sulfonyl]phenyl}methyl)-1-piperazinyl]-3-pyridine carboxylate;
1-methylethyl2-{4-[(4-{R{2-[(dimethylamino)sulfonyl]phenyl}methyl)(ethyl)amino)methyl}phenyl)methyl]-1-piperazinyl}-3-pyridinecarboxylate;
1-methylethyl2-{4-[(4-{R{3-[(dimethylamino)sulfonyl]phenyl}methyl)(ethyl)amino)methyl}phenyl)methyl]-1-piperazinyl}-3-pyridinecarboxylate; 1-methylethyl2-{4-[(4-{R{4[(dimethylamino)sulfonyl]phenyl}methyl)(ethyl)amino)methyl}phenyl)methyl]-1-piperazinyl}-3-pyridinecarboxylate; 1-Methylethyl 2-{4-[(4-{[[2-(2-chloro-6-fluorophenyl)ethyl](ethyl)amino]methyl}phenyl)methyl]-1-piperazinyl}-3-pyridinecarboxylate dihydrochloride;
1-methylethyl 2-{4-[(4-{[[(2-chloro-6-fluorophenyl)methyl](ethyl)amino]methyl}phenyl)methyl]-1-piperazinyl}-3-pyridinecarboxylate dihydrochloride hydrochloride; 1-methylethyl 2-(4-{[4-({ethyl[(3-fluorophenyl)methyl]amino}methyl)phenyl]methyl}-1-piperazinyl)-3-pyridinecarboxylate;
1-methylethyl 2-(4-{[4-({ethyl[(4-fluorophenyl)methyl]amino}methyl)phenyl]methyl}-1-piperazinyl)-3-pyridinecarboxylate;
1-methylethyl2-{4-[(4-{[[(2,6-difluorophenyl)methyl](ethyl)amino]methyl}phenyl)methyl]-1-piperazinyl}-3-pyridinecarboxylate; 1-methylethyl 2-(4-{[4-({ethyl[(2-fluorophenyl)methyl]amino}methyl)phenyl]methyl}-1-piperazinyl)-3-pyridinecarboxylate; 1-methylethyl2-{4-[(4-{[[(2,6-dichlorophenyl)methyl](ethyl)amino]methyl}phenyl)methyl]-1-piperazinyl}-3-pyridinecarboxylate; 1-methylethyl 2-{4-[(4-{[[(3-chlorophenyl)methyl](ethyl)amino]methyl}phenyl)methyl]-1-piperazinyl}-3-pyridinecarboxylate; 1-methylethyl2-{4-[4-(4-{[ethyl(phenylmethyl]amino}methyl)}phenyl)methyl]-1-piperazinyl)-3-pyridine carboxylate;
1-methylethyl2-{4-[(4-{[[(4-chlorophenyl)methyl](ethyl)amino]methyl}phenyl)methyl]-1-piperazinyl}-3-pyridinecarboxylate;
1-methylethyl2-{4-[(4-{[[(2-chlorophenyl)methyl](ethyl)amino]methyl}phenyl)methyl]-1-piperazinyl}-3-pyridinecarboxylate;
1-methylethyl2-(4-{[4-({ethyl[(6-methyl-2-pyridinyl)methyl]amino}methyl)phenyl]methyl}-1-piperazinyl)-3-pyridinecarboxylate;
1-methylethyl2-(4-{[4-({[(2-chloro-6-fluorophenyl)methyl]amino}methyl)phenyl]methyl}-1-piperazinyl)-3-pyridinecarboxylate;
1-methylethyl2-{4-[(4-{[[(2-chloro-6-fluorophenyl)carbonyl](ethyl)amino]methyl}phenyl)methyl]-1-piperazinyl}-3-pyridinecarboxylate;
1-methylethyl2-(4-{[4-({[(2-chloro-6-fluorophenyl)methyl][3-(2-oxo-1-pyrrolidinyl)propyl]amino}methyl)phenyl]methyl}-1-piperazinyl)-3-pyridinecarboxylate;
1-methylethyl2-{4-[(3-{[[(2-chloro-6-fluorophenyl)methyl](ethyl)amino]methyl}phenyl)methyl]-1-piperazinyl}-3-pyridinecarboxylate;
1-methylethyl2-(4-{[4-({ethyl[(2-methyl-3-pyridinyl)methyl]amino}methyl)phenyl]methyl}-1-piperazinyl)-3-pyridinecarboxylate;
1-methylethyl2-(4-{[4-({[(2-fluorophenyl)methyl]amino}methyl)phenyl]methyl}-1-piperazinyl)-3-pyridinecarboxylate;
1-methylethyl2-{4-[(2-{[[(2-chloro-6-fluorophenyl)methyl](ethyl)amino]; methyl]phenyl)methyl}-1-piperazinyl}-3-pyridinecarboxylate;
1-methylethyl2-{4-[(4-{[[3-(2-chloro-6-fluorophenyl)propyl](ethyl)amino]methyl}phenyl)methyl]-1-piperazinyl}-3-pyridinecarboxylate;
1-methylethyl 2-{4-[(4-{[(phenylmethyl)amino]methyl}phenyl)methyl]-1-piperazinyl}_-3-pyridinecarboxylate;
1-methylethyl 2-(4-{[4-({ethyl[(2-fluorophenyl)methyl]amino}methyl)phenyl]methyl}-1-piperazinyl)-3-pyridinecarboxylate dihydrochloride;
1-methylethyl2-{4-[(4-{[ethyl(phenylmethyl)amino]methyl}phenyl)methyl]-1-piperazinyl}-3-pyridine carboxylate dihydrochloride;
1-methylethyl2-(4-{[4-({[(2-chloro-6-fluorophenyl)carbonyl]amino}methyl)phenyl]methyl}-1-piperazinyl)-3-pyridinecarboxylate;
1-methylethyl2-(4-{[4-({ethyl[(6-methyl-2-pyridinyl)methyl]amino}methyl)phenyl]methyl}-1-piperazinyl)-3-pyridine carboxylate quaternary hydrochloride1-methylethyl2-(4-{[4-({[(2-fluorophenyl)carbonyl]amino}methyl)phenyl}methyl]-1-piperazinyl)-3-pyridinecarboxylate;
1-methylethyl 2-{4-[(4-{[(phenylcarbonyl)amino]methyl}phenyl)methyl]-1-piperazinyl}-3-pyridinecarboxylate;
1-methylethyl 2-(4-{[4-({[(2-chloro-6-fluorophenyl)methyl]oxy}methyl)phenyl]methyl}-1-piperazinyl)-3-pyridinecarboxylate;
1-methylethyl2-(4-{[4-({[(2-chloro-6-fluorophenyl)methyl]amino}methyl)phenyl]methyl}-1-piperazinyl)-3-pyridine carboxylate trihydrochloride; 1-methylethyl2-{4-[(4-{[[(2-chloro-6-fluorophenyl)carbonyl](ethyl)amino]methyl}phenyl)methyl]-1-piperazinyl}-3-pyridinecarboxylate dihydrochloride; or
a pharmaceutically acceptable salt thereof.

8. A compound which is:
1-methylethyl 2-{4-[(4-{[[(2-chloro-6-fluorophenyl)methyl](ethyl)amino]methyl}phenyl)methyl]-1-piperazinyl}-3-pyridinecarboxylate;
bis(1-methylethyl) 2,2′-{benzene-1,4-diylbis[methanediyl(ethylimino)(3S)-3,1-pyrrolidinediyl]}(3-pyridinecarboxylate);
benzene-1,4-diylbis[methanediyl(ethylimino)(3R)-3,1-pyrrolidinediyl-2,3-pyridinediylmethanediyl]bis(3,3-dimethylbutanoate; 1-methylethyl 2-{4-[(3-{[(2-chloro-6-fluorophenyl)methyl]oxy}phenyl)methyl]-1-piperazinyl}-3-pyridinecarboxylate;
1-methylethyl 2-{4-[(3-{[4-(methyloxy)phenyl]oxy}phenyl)methyl]-1-piperazinyl}-3-pyridinecarboxylate;
1-methylethyl 2-(4-{[Z-(trifluoromethyl)-3-biphenylyl]methyl}-1-piperazinyl)-3-pyridinecarboxylate;
1-methylethyl 2-(4-{[3-({[2-(methyloxy)phenyl]methyl}oxy)phenyl]methyl}-1-piperazinyl)-3-pyridinecarboxylate;
1-[(4-{[[(2-Chloro-6-fluorophenyl)methyl](ethyl)ammonio]methyl}phenyl)methyl]-4-(3-{[(1-methylethyl)oxy]carbonyl}-2-pyridinyl)piperazin-1-ium di-maleate; 1-methylethyl 2-(4-{[3-({[4-(ethyloxy)phenyl]methyl}oxy)phenyl}methyl]-1-piperazinyl)-3-pyridinecarboxylate;
1-methylethyl 2-[(3R)-3-(ethyl{[4-({ethyl[(3R)-1-(1-{2-[(1-methylethyl)oxy]-2-oxoethyl]ethenyl)-3-pyrrolidinyl]amino}methyl)phenyl}methyl}amino)-1-pyrrolidinyl]-3-pyridinecarboxylate;
1-methylethyl 2-(4-{[3-({[3-(methyloxy)phenyl]methyl}oxy)phenyl]methyl}-1-piperazinyl)-3-pyridinecarboxylate;
1-methylethyl 2-{(3R)-3-[ethyl({2-[(phenylmethyl)oxy]phenyl}methyl)amino]-1-pyrrolidinyl}-3-pyridinecarboxylate; bis(1-methylethyl) 2,2′-{benzene-1,4-diylbis[methanediyl(2S)-1,2-pyrrolidinediylmethanediyloxy]}di(3-pyridinecarboxylate); or
pharmaceutically acceptable salts thereof.

9. A compound which is 1-Methylethyl 2-{4-[(4-{[[(2-chloro-6-fluorophenyl)methyl](ethyl)amino]methyl}phenyl)methyl]-1-piperazinyl}-3-pyridinecarboxylate 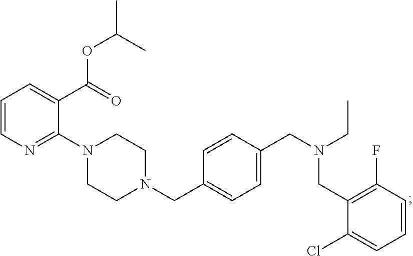 [SMILES: CCN(Cc1ccc(CN2CCN(c3ncccc3C(=O)OC(C)C)CC2)cc1)Cc1c(F)cccc1Cl ; ";"]
or
a pharmaceutically acceptable salt thereof.

10. A compound which is 1-Methylethyl 2-{4-[(4-{[[(2-chloro-6-fluorophenyl)methyl](ethyl)amino]methyl}phenyl)methyl]-1-piperazinyl}-3-pyridinecarboxylate

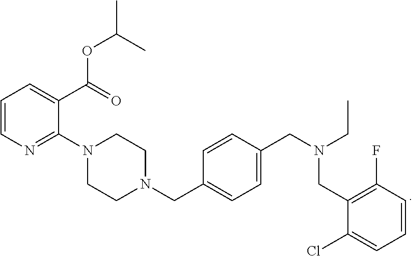 [SMILES: CCN(Cc1ccc(CN2CCN(c3ncccc3C(=O)OC(C)C)CC2)cc1)Cc1c(F)cccc1Cl ; "."]

11. A compound which is 1-[(4-{[[(2-Chloro-6-fluorophenyl)methyl](ethyl) ammonio]methyl}phenyl)methyl]-4-(3-{[(1-methylethyl)oxy]carbonyl}-2-pyridinyl)piperazin-1-ium di-maleate

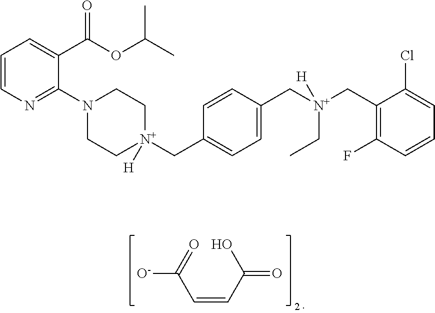

12-29. (canceled)

30. A pharmaceutical composition, which comprises a compound according to claim 6 or a pharmaceutically acceptable salt thereof and at least one pharmaceutically acceptable adjuvant, excipient or carrier.

31. A pharmaceutical composition, which comprises a compound according to claim 9 or a pharmaceutically acceptable salt thereof and at least one pharmaceutically acceptable adjuvant, excipient or carrier.

32. A pharmaceutical composition, which comprises a compound or a pharmaceutically acceptable salt thereof according to claim 11 and at least one pharmaceutically acceptable adjuvant, excipient or carrier.

33. A method for treatment of respiratory or respiratory tract diseases, which comprises administering an effective amount of a compound according to claim 6 to a subject in need thereof.

34. (canceled)

35. A method for treatment of respiratory or respiratory tract diseases, which comprises administering an effective amount of a pharmaceutical composition according to claim 31 to a subject in need thereof.

36. The method according to claim 35, wherein the respiratory or respiratory tract diseases are selected from asthma, allergen-induced asthmatic reactions, cystic fibrosis, bronchitis, chronic bronchitis, chronic obstructive pulmonary disease (COPD), cough, adult respiratory distress syndrome (ARDS), chronic pulmonary inflammation, rhinitis and upper respiratory tract inflammatory disorders (URID), ventilator induced lung injury, silicosis, pulmonary sarcoidosis, idiopathic pulmonary fibrosis or bronchopulmonary dysplasia.

37. A method for treatment of chronic obstructive pulmonary diseases (COPD), which comprises administering an effective amount of a compound according to claim 6 to a subject in need thereof.

38. A method for treatment of chronic obstructive pulmonary diseases (COPD), which comprises administering an effective amount of a pharmaceutical composition according to claim 30 to a subject in need thereof.

39. A method for treatment of cough, which comprises administering an effective amount of a compound according to claim 6 to a subject in need thereof.

40. A method for treatment of cough, which comprises administering an effective amount of a pharmaceutical composition according to claim 30 to a subject in need thereof.

41-44. (canceled)

45. A method for treatment of chronic obstructive pulmonary diseases (COPD), which comprises administering an effective amount of a compound according to claim 9 to a subject in need thereof.

46. A method for treatment of chronic obstructive pulmonary diseases (COPD), which comprises administering an effective amount of a compound according to claim 11 to a subject in need thereof.

47. A method for treatment of chronic obstructive pulmonary diseases (COPD), which comprises administering an effective amount of a pharmaceutical composition according to claim 31 to a subject in need thereof.

48. A method for treatment of cough, which comprises administering an effective amount of a compound according to claim 9 to a subject in need thereof.

49. The method for treatment of cough according to claim 49, wherein the cough is dry cough, wet cough, croupy cough or chest cough.

50. A method for treatment of cough, which comprises administering an effective amount of a pharmaceutical composition according to claim 31 to a subject in need thereof.

51. The method for treatment of cough according to claim 51, wherein the cough is dry cough, wet cough, croupy cough or chest cough.
